(19) United States
(12) Patent Application Publication
(10) Pub. No.: US 2018/0162862 A1
(43) Pub. Date: Jun. 14, 2018

(54) INHIBITORS OF BACTERIAL GLYCOSYL TRANSFERASES

(71) Applicants: President and Fellows of Harvard College, Cambridge, MA (US); Evotec International GmbH, Hamburg (DE)

(72) Inventors: Christian M. Gampe, Brighton, MA (US); Daniel Evan Kahne, Brookline, MA (US); Suzanne Walker Kahne, Brookline, MA (US); Yuan Qia Cambridge, Cambrige, MA (US); Stephen East, Wallingford, Oxfordshire (GB); Alastair L. Parkes, Caversham, Reading, Berkshire (GB); Michelle South, Uffington, Faringdon, Oxfordshire (GB); James Hunter, Earley, Reading, Berkshire (GB); Mark Whittaker, Abingdon, Oxfordshire (GB); Martin Arthuis, Abingdon, Oxfordshire (GB)

(73) Assignees: President and Fellows of Harvard College, Cambridge, MA (US); Evotec International GmbH, Hamburg (DE)

(21) Appl. No.: 15/577,256

(22) PCT Filed: May 27, 2016
(86) PCT No.: PCT/US2016/034587

§ 371 Date: Nov. 27, 2017

Related U.S. Application Data
(60) Provisional application No. 62/167,813, filed on May 28, 2015.

(51) Int. Cl. C07D 487/04; A61P 31/04; A61K 45/06; A61K 31/53; A01N 43/90

(52) U.S. Cl. CPC C07D 487/04; A61P 31/04; A01N 43/90; A61K 31/53; A61K 45/06

(57) ABSTRACT

Described herein are compounds of Formula (I′), Formula (IA), Formulae (I)-(VII), pharmaceutically acceptable salts, solvates, hydrates, polymorphs, co-crystals, tautomers, stereoisomers, isotopically labeled derivatives, and prodrugs thereof. The invention also provides pharmaceutical compositions of the compounds for human and veterinary use. Compounds of the present invention are useful for inhibiting bacterial growth and therefore are useful in treating and/or preventing bacterial infections. Methods of using the compounds for treating and/or preventing a bacterial infection in a subject are also described.

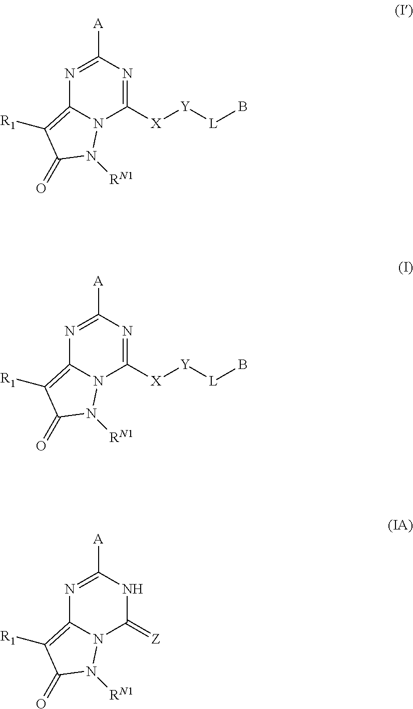

### RELATED APPLICATIONS

This application claims priority under 35 U.S.C. § 119(e) to U.S. Provisional Application, U.S. Ser. No. 62/167,813, filed May 28, 2015, which is incorporated herein by reference.

### GOVERNMENT FUNDING

This invention was made with government support under grant numbers GM066174, GM076710, AI083214, and AI057159, awarded by the National Institutes of Health. The government has certain rights in the invention.

### BACKGROUND OF THE INVENTION

The supporting structure of the bacterial cell wall is a layer of polysaccharide strands containing peptide cross bridges, termed peptidoglycan (PG). This polymer protects the cell membrane from rupture in harsh environments. The final stage of the extracellular biosynthesis of PG proceeds in two steps: in the transglycosylation step, the disaccharide phospholipid lipid II is polymerized to form polysaccharide strands, and in the subsequent transpeptidation step these strands are crosslinked (Van Heijenoort et al., Glycobiology, 2001, 11, p 25R-36R; Lovering et al., Annu. Rev. Biochem. 2012, 81, p 451; Vollmer et al., U. Biochim. Biophys. Acta, 2008, 1778, p 1714; Vollmer et al., FEMS Microbiol. Rev., 2008, 32, p 149). These transformations are catalyzed by bifunctional penicillin binding proteins (PBPs) that have both a glycosyltransferase (GT) and a transpeptidase (TP) active site (Sauvage et al., FEMS Microbiol. Rev. 2008, 32, p 234). Additionally, some bacteria possess monofunctional peptidoglycan glycosyltransferases (PGTs) that form polysaccharide strands, which are then crosslinked by PBPs (Wang et al., J. Am. Chem. Soc., 2008, 130, p 14068-14069). In bacteria, proper synthesis of PG is required for cell viability, and inhibition of PG synthesis leads to cell death. Therefore, efforts devoted to the development of new antibiotics have focused on targets involved in cell wall synthesis and remodelling (Walsh et al., In Antibiotics: Actions, Origins, Resistance; ASM Press: Washington, D.C., 2003, p 1-9; Silver et al., Clin. Microbiol. Rev., 2011, p 71-109).

Bacteria have the ability to generate resistance to antibiotics through lateral gene transfer, mutation of enzymes, or the expression of enzymes which actively pump the antibiotic out of the cell or break it down (Walsh et al., In Antibiotics: Actions, Origins, Resistance; ASM Press: Washington, D.C., 2003, p 1-9; Silver et al., Clin. Microbiol. Rev., 2011, p 71-109). Over the past 10 years, resistance to existing antibiotics has become a significant problem. In practice, strains such as methicillin-resistant Staphylococcus aureus (MRSA), methicillin-resistant Staphylococcus epidermidis (MRSE), penicillin-resistant Streptococcus pneumonia, quinolone-resistant Staphylococcus aureus (QRSA), vancomycin-resistant Staphylococcus aureus (VRSA), vancomycin-resistant Enterococci (VRE), and multi-drug resistant Mycobacterium tuberculosis show resistance to most antibiotics in use. Thus, the developments of new antibiotics to overcome resistant organisms are needed.

### SUMMARY OF THE INVENTION

Peptidoglycan (PG), also known as murein, is a polymer consisting of sugars and amino acids that forms the supporting structures of the bacteria cell wall. Some bacteria possess monofunctional peptidoglycan glycosyltransferases (PGTs) to form polysaccharide strands, which are then crosslinked by penicillin binding proteins (PBPs). Inhibition of PGT activities presents a promising target for developing novel antibiotics.

The present invention provides compounds of Formula (I′), Formula (IA), Formulae (I)-(VII), pharmaceutical compositions thereof, and kits including the compounds and compositions described herein useful in inhibiting glycosyltransferases, e.g., peptidoglycan glycosyltransferases. The present invention further provides methods of using the inventive compounds, and pharmaceutically acceptable salts, solvates, hydrates, polymorphs, co-crystals, tautomers, stereoisomers, isotopically labeled derivatives, and prodrugs thereof, and compositions thereof, to treat and prevent infectious diseases (e.g., bacterial infections).

In one aspect, the present invention provides compounds of Formula (I′):

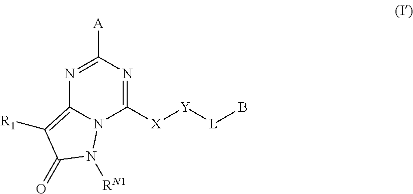

and pharmaceutically acceptable salts, solvates, hydrates, polymorphs, co-crystals, tautomers, stereoisomers, isotopically labeled derivatives, and prodrugs thereof, wherein R₁, R^{N1}, A, B, L, X, and Y are as defined herein. In Formula (I′), X is not S, and A may be optionally substituted C₂₋₆ alkyl or optionally substituted carbocyclyl.

In some embodiments, the present invention provides compounds of Formula (I′-i-A):

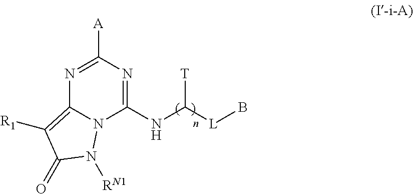

and pharmaceutically acceptable salts, solvates, hydrates, polymorphs, co-crystals, tautomers, stereoisomers, isotopically labeled derivatives, and prodrugs thereof, wherein R₁, R^{N1}, A, B, T, L, B, and n are as defined herein.

In some embodiments, the present invention provides compounds of Formula (I′-i-B):

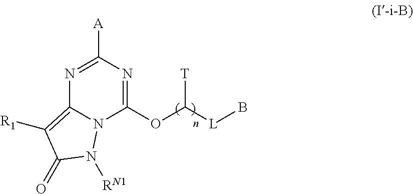

and pharmaceutically acceptable salts, solvates, hydrates, polymorphs, co-crystals, tautomers, stereoisomers, isotopically labeled derivatives, and prodrugs thereof, wherein R₁, R^{N1}, A, B, T, L, B, and n are as defined herein.

In some embodiments, the present invention provides compounds of Formula (I′-i-C):

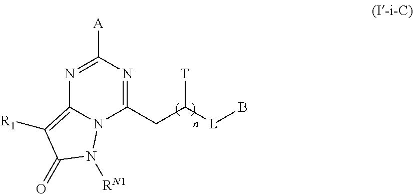

and pharmaceutically acceptable salts, solvates, hydrates, polymorphs, co-crystals, tautomers, stereoisomers, isotopically labeled derivatives, and prodrugs thereof, wherein R₁, R^{N1}, A, B, T, L, B, and n are as defined herein.

In one aspect, the present invention provides compounds of Formula (I):

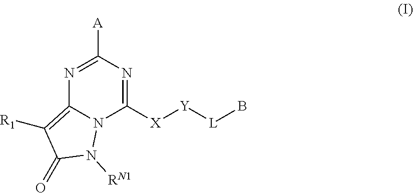

and pharmaceutically acceptable salts, solvates, hydrates, polymorphs, co-crystals, tautomers, stereoisomers, isotopically labeled derivatives, and prodrugs thereof, wherein R₁, R^{N1}, A, B, L, X, and Y are as defined herein.

In some embodiments, the present invention provides compounds of Formula (I-i):

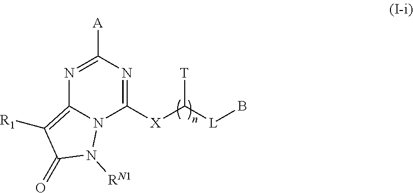

and pharmaceutically acceptable salts, solvates, hydrates, polymorphs, co-crystals, tautomers, stereoisomers, isotopically labeled derivatives, and prodrugs thereof, wherein R₁, R^{N1}, A, B, X, T, L, B, and n are as defined herein.

In some embodiments, the present invention provides compounds of Formula (I-ii):

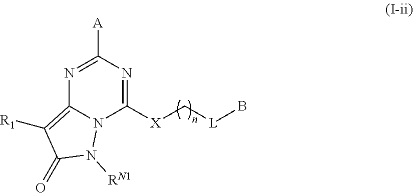

and pharmaceutically acceptable salts, solvates, hydrates, polymorphs, co-crystals, tautomers, stereoisomers, isotopically labeled derivatives, and prodrugs thereof, wherein R₁, R^{N1}, A, B, X, L, B, and n are as defined herein.

In some embodiments, the present invention provides compounds of Formula (I-a):

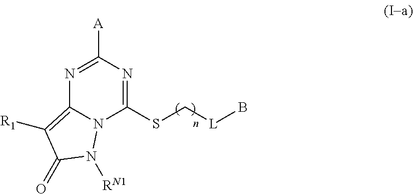

and pharmaceutically acceptable salts, solvates, hydrates, polymorphs, co-crystals, tautomers, stereoisomers, isotopically labeled derivatives, and prodrugs thereof, wherein R₁, R^{N1}, A, B, L, and n are as defined herein.

In some embodiments, the present invention provides compounds of Formula (I-b):

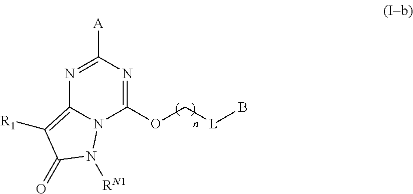

and pharmaceutically acceptable salts, solvates, hydrates, polymorphs, co-crystals, tautomers, stereoisomers, isotopically labeled derivatives, and prodrugs thereof, wherein R₁, R^{N1}, A, B, L, and n are as defined herein.

In some embodiments, the present invention provides compounds of Formula (I-c):

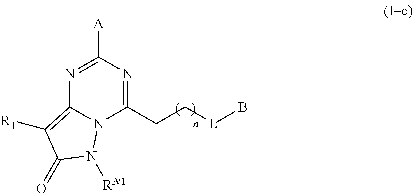

and pharmaceutically acceptable salts, solvates, hydrates, polymorphs, co-crystals, tautomers, stereoisomers, isotopically labeled derivatives, and prodrugs thereof, wherein R₁, R^{N1}, A, B, L, and n are as defined herein.

In some embodiments, the present invention provides compounds of Formula (I-d):

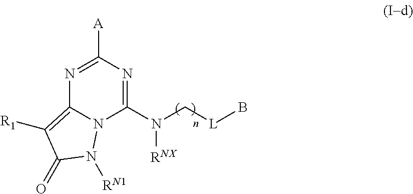

and pharmaceutically acceptable salts, solvates, hydrates, polymorphs, co-crystals, tautomers, stereoisomers, isotopically labeled derivatives, and prodrugs thereof, wherein R₁, R^{N1}, R^{NX}, A, B, L, and n are as defined herein.

In some embodiments, the present invention provides compounds of Formula (IA):

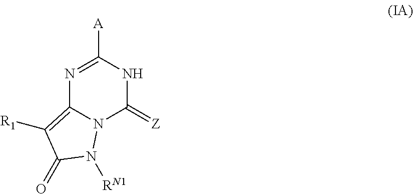

and pharmaceutically acceptable salts, solvates, hydrates, polymorphs, co-crystals, tautomers, stereoisomers, isotopically labeled derivatives, and prodrugs thereof, wherein R₁, R^{N1}, A, and Z are as defined herein.

In some embodiments, the present invention provides compounds of Formula (II):

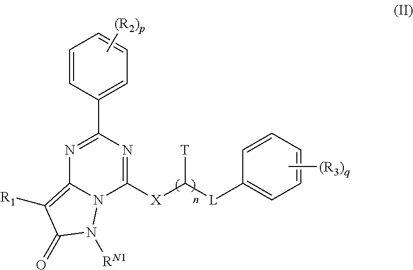

and pharmaceutically acceptable salts, solvates, hydrates, polymorphs, co-crystals, tautomers, stereoisomers, isotopically labeled derivatives, and prodrugs thereof, wherein R₁, R₂, R₃, R^{N1}, X, L, T, n, p, and q are as defined herein.

In some embodiments, the present invention provides compounds of Formula (III):

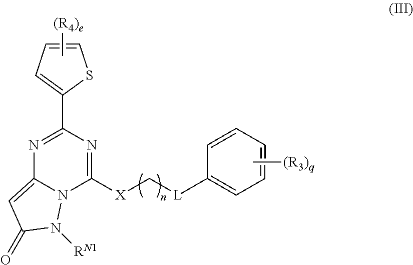

and pharmaceutically acceptable salts, solvates, hydrates, polymorphs, co-crystals, tautomers, stereoisomers, isotopically labeled derivatives, and prodrugs thereof, wherein R₃, R₄, R^{N1}, X, L, n, e, and q are as defined herein.

In some embodiments, the present invention provides compounds of Formula (IV):

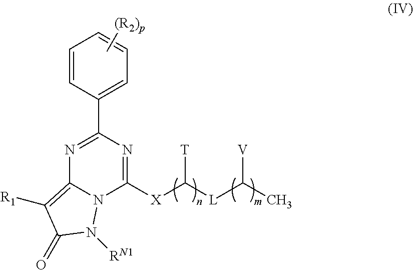

and pharmaceutically acceptable salts, solvates, hydrates, polymorphs, co-crystals, tautomers, stereoisomers, isotopically labeled derivatives, and prodrugs thereof, wherein R₁, R₂, R^{N1}, X, L, T, V, n, p, and m are as defined herein.

In yet another aspect, the present invention provides compounds of Formula (V):

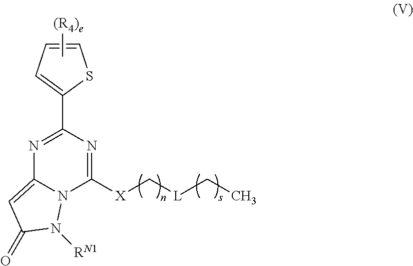

and pharmaceutically acceptable salts, solvates, hydrates, polymorphs, co-crystals, tautomers, stereoisomers, isotopically labeled derivatives, and prodrugs thereof, wherein R₄, R^{N1}, X, L, n, e, and s are as defined herein.

In yet another aspect, the present invention provides compounds of Formula (VI):

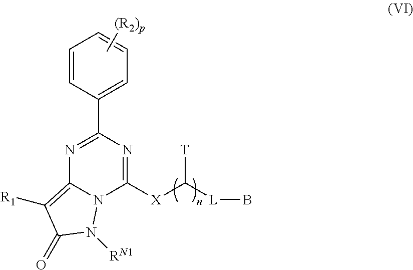

and pharmaceutically acceptable salts, solvates, hydrates, polymorphs, co-crystals, tautomers, stereoisomers, isotopically labeled derivatives, and prodrugs thereof, wherein R₁, R₂, R^{N1}, X, L, T, B, n, and p are as defined herein.

In yet another aspect, the present invention provides compounds of Formula (VII):

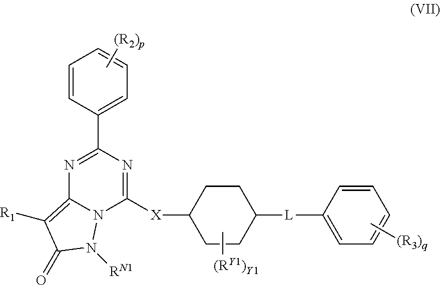

and pharmaceutically acceptable salts, solvates, hydrates, polymorphs, co-crystals, tautomers, stereoisomers, isotopically labeled derivatives, and prodrugs thereof, wherein R₁, R₂, R₃, R^{N1}, X, L, R^{Y1}, Y1, p, and q are as defined herein.

In another aspect, the present invention provides methods of synthesizing a compound of any one of Formula (I′), Formula (IA), Formulae (I)-(VII), or a pharmaceutically acceptable salt, solvate, hydrate, polymorph, co-crystal, tautomer, stereoisomer, isotopically labeled derivative, or prodrug thereof.

In another aspect, the present invention provides pharmaceutical compositions comprising a compound of any one of Formula (I′), Formula (IA), Formulae (I)-(VII), or a pharmaceutically acceptable salt, solvate, hydrate, polymorph, co-crystal, tautomer, stereoisomer, isotopically labeled derivative, or prodrug thereof, and optionally a pharmaceutically acceptable carrier. In certain embodiments, the pharmaceutical compositions described herein include a therapeutically effective amount of a compound of Formula (I′), Formula (IA), Formula (I)-(VII), or a pharmaceutically acceptable salt, solvate, hydrate, polymorph, co-crystal, tautomer, stereoisomer, isotopically labeled derivative, or prodrug thereof. The compounds described herein are useful for inhibiting glycosyltransferases, and therefore, may be useful in the treatment and prevention of infectious disease such as bacterial infections. In certain embodiments, the pharmaceutical compositions described herein are useful for inhibiting peptidoglycan glycosyltransferases. In another embodiment, the pharmaceutical compositions described herein are useful for inhibiting bacterial growth and/or treating or preventing bacterial infections. In another embodiment, the pharmaceutical compositions described herein are useful for killing bacteria.

In certain embodiments, the compounds described herein have antibacterial activity against Gram-positive bacteria. In certain embodiments, the compounds described herein have antibacterial activity against Gram-negative bacteria. In certain embodiments, the compounds described herein have antibacterial activity against at least one species selected from the group consisting of Staphylococcus sp., Enterococcus sp., Escherichia coli, Bacillus sp., Salmonella sp., and Mycobacterium sp. In certain embodiments, the compounds described herein have antibacterial activity against at least one species selected from the group consisting of Staphylococcus, Streptococcus, Micrococcus, Peptococcus, Peptostreptococcus, Enterococcus, Bacillus, Clostridium, Lactobacillus, Listeria, Erysipelothrix, Propionibacterium, Eubacterium, Corynebacterium, Capnocytophaga, Bifidobacterium, and Gardnerella. In certain embodiments, the compounds described herein have antibacterial activity against at least one species selected from the group consisting of Escherichia, Citrobacter, Enterobacter, Klebsiella, Proteus, Serratia, Shigella, Salmonella, Morganella, Providencia, Edwardsiella, Erwinia, Hafnia, Yersinia, Acinetobacter, Vibrio, Aeromonas, Pseudomonas, Haemophilus, Pasteurella, Campylobacter, Helicobacter, Branhamella, Moraxella, Neisseria, Veillonella, Fusobacterium, Bacteroides, Actinobacillus, Aggregatibacter, Agrobacterium, Porphyromonas, Prevotella, Ruminobacter, Roseburia, Caulobacter, Francisella, Borrelia, Treponema, Brucella, and Rickettsia. In certain embodiments, the compounds described herein have antibacterial activity against at least one species selected from the group consisting of Escherichia coli, Morganella morganii, Branhamella catarrhalis, Veillonella parvula, Actinobacillus actinomycetemcomitans, Aggregatibacter actinomycetemcomitans, Caulobacter crescentus, and Treponema pallidum. In certain embodiments, the pharmaceutical compositions are also useful for treating or preventing drug-resistant bacterial infections. In certain embodiments, the bacteria are selected from methicillin-resistant Staphylococcus aureus (MRSA), methicillin-resistant Staphylococcus epidermidis (MRSE), penicillin-resistant Streptococcus pneumonia, quinolone-resistant Staphylococcus aureus (QRSA), vancomycin-resistant Staphylococcus aureus (VRSA), vancomycin-resistant Enterococci (VRE), and multi-drug resistant Mycobacterium tuberculosis has.

In another aspect, the present invention provides methods for inhibiting bacterial growth comprising administering to a subject a therapeutically effective amount of a compound of any one of Formula (I′), Formula (IA), Formulae (I)-(VII), or a pharmaceutically acceptable salt, solvate, hydrate, polymorph, co-crystal, tautomer, stereoisomer, isotopically labeled derivative, or prodrug thereof. In another aspect, the present invention provides methods for killing bacteria comprising administering to a subject a therapeutically effective amount of a compound of any one of Formula (I′), Formula (IA), Formulae (I)-(VII), or a pharmaceutically acceptable salt, solvate, hydrate, polymorph, co-crystal, tautomer, stereoisomer, isotopically labeled derivative, or prodrug thereof. In certain embodiments, the present invention is a bactericidal antibiotic. In certain embodiments, the present invention is a bacteriostatic antibiotic. In another aspect, the present invention provides methods for inhibiting bacterial infections comprising administering to a subject a therapeutically effective amount of a compound of any one of Formula (I′), Formula (IA), Formulae (I)-(VII), or a pharmaceutically acceptable salt, solvate, hydrate, polymorph, co-crystal, tautomer, stereoisomer, isotopically labeled derivative, or prodrug thereof. In certain embodiments, the bacterium is selected from the group consisting of Staphylococcus, Streptococcus, Micrococcus, Peptococcus, Peptostreptococcus, Enterococcus, Bacillus, Clostridium, Lactobacillus, Listeria, Erysipelothrix, Propionibacterium, Eubacterium, Corynebacterium, Capnocytophaga, Bifidobacterium, and Gardnerella. In certain embodiments, the bacterium is selected from the group consisting of Staphylococcus sp., Enterococcus sp., Escherichia coli, Bacillus sp., Salmonella sp., and Mycobacterium sp. In certain embodiments, the bacterium is selected from the group consisting of Escherichia, Citrobacter, Enterobacter, Klebsiella, Proteus, Serratia, Shigella, Salmonella, Morganella, Providencia, Edwardsiella, Erwinia, Hafnia, Yersinia, Acinetobacter, Vibrio, Aeromonas, Pseudomonas, Haemophilus, Pasteurella, Campylobacter, Helicobacter, Branhamella, Moraxella, Neisseria, Veillonella, Fusobacterium, Bacteroides, Actinobacillus, Aggregatibacter, Agrobacterium, Porphyromonas, Prevotella, Ruminobacter, Roseburia, Caulobacter, Francisella, Borrelia, Treponema, Brucella, and Rickettsia. In certain embodiments, the bacterium is selected from the group consisting of Escherichia coli, Morganella morganii, Branhamella catarrhalis, Veillonella parvula, Actinobacillus actinomycetemcomitans, Aggregatibacter actinomycetemcomitans, Caulobacter crescentus, and Treponema pallidum. In certain embodiments, the infection being treated or prevented is caused by methicillin-resistant Staphylococcus aureus (MRSA), methicillin-resistant Staphylococcus epidermidis (MRSE), penicillin-resistant Streptococcus pneumonia, quinolone-resistant Staphylococcus aureus (QRSA), vancomycin-resistant Staphylococcus aureus (VRSA), vancomycin-resistant Enterococci (VRE), or multi-drug resistant Mycobacterium tuberculosis.

In another aspect, the present invention provides kits comprising a compound of any one of Formula (I′), Formula (IA), Formulae (I)-(VII), or a pharmaceutically acceptable salt, solvate, hydrate, polymorph, co-crystal, tautomer, stereoisomer, isotopically labeled derivative, or prodrug thereof, or a pharmaceutical composition thereof. The kits of the invention may include a single dose or multiple doses of a compound of any one of Formula (I′), Formula (IA), Formulae (I)-(VII), or a pharmaceutically acceptable salt, solvate, hydrate, polymorph, co-crystal, tautomer, stereoisomer, isotopically labeled derivative, or prodrug thereof, or a pharmaceutical composition thereof. The provided kits may be useful for the treatment or prevention of bacterial infections. The provided kits may also be useful for the treatment of diseases or disorders associated with drug-resistant bacterial infections. In certain embodiments, the kits described herein further include instructions for administering a compound of any one of Formula (I′), Formula (IA), Formulae (I)-(VII), or a pharmaceutically acceptable salt, solvate, hydrate, polymorph, co-crystal, tautomer, stereoisomer, isotopically labeled derivative, or prodrug thereof, or a pharmaceutical composition thereof. The kits may also include packaging information describing the use or prescribing information for the subject or a health care professional. Such information may be required by a regulatory agency such as the U.S. Food and Drug Administration (FDA). The kit may also optionally include a device for administration of the compound or composition, for example, a syringe for parenteral administration.

The details of certain embodiments of the invention are set forth herein. Other features, objects, and advantages of the invention will be apparent from the Detailed Description, the Figures, the Examples, and the Claims.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

When a range of values is listed, it is intended to encompass each value and sub-range within the range. For example “C₁₋₆” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

“Alkyl” refers to a radical of a straight-chain or branched saturated hydrocarbon group having from 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), iso-propyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents. In certain embodiments, the alkyl group is unsubstituted C₁₋₁₀ alkyl (e.g., —CH₃). In certain embodiments, the alkyl group is substituted C₁₋₁₀ alkyl.

“Alkenyl” refers to a radical of a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkynyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), 15ydroxy[2.2.1]heptanyl (C₇), 15ydroxy[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused to one or more aryl or heteroaryl groups wherein the point of attachment is on the carbocyclic ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₆). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In certain embodiments, the heteroatom is independently selected from nitrogen, sulfur, and oxygen. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is either on the carbocyclyl or heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, aziridinyl, oxiranyl, and thiorenyl. Exemplary 4-membered heterocyclyl groups containing one heteroatom include, without limitation, azetidinyl, oxetanyl, and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl, and thiepanyl. Exemplary 8-membered heterocyclyl groups containing one heteroatom include, without limitation, azocanyl, oxecanyl, and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Arylalkyl” or “aralkyl” refers to a subset of alkyl and aryl, as defined herein, and refers to an optionally substituted alkyl group substituted by an optionally substituted aryl group. In certain embodiments, the aralkyl is optionally substituted benzyl. In certain embodiments, the aralkyl is benzyl. In certain embodiments, the aralkyl is optionally substituted phenethyl. In certain embodiments, the aralkyl is phenethyl.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen, and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” is a subset of alkyl and heteroaryl, as defined herein, and refers to an optionally substituted alkyl group substituted by an optionally substituted heteroaryl group.

“Partially unsaturated” refers to a group that includes at least one double or triple bond. A “partially unsaturated” ring system is further intended to encompass rings having multiple sites of unsaturation, but is not intended to include aromatic groups (e.g., aryl or heteroaryl groups) as herein defined. Likewise, “saturated” refers to a group that does not contain a double or triple bond, i.e., it contains all single bonds.

Alkyl, alkenyl, alkynyl, carbocyclyl, heterocyclyl, aryl, and heteroaryl groups, as defined herein, which are divalent bridging groups are further referred to using the suffix—ene, e.g., alkylene, alkenylene, alkynylene, carbocyclylene, heterocyclylene, arylene, and heteroarylene.

As used herein, the term “optionally substituted” refers to a substituted or unsubstituted moiety.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —O(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}—, —C(═NR^{bb})OR^{aa}—, —OC(═NR^{bb})R^{aa}—, —OC(═NR^{bb})OR^{aa}—, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}—, —NR^{bb}SO₂R^{aa}, —SO₂N(R^{bb})₂, —SO₂R^{aa}, —SO₂OR^{aa}, —OSO₂R^{aa}, —S(═O)R^{aa}—, —OS(═O)R^{aa}—, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R_{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}—, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}—, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}S₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆ alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

Exemplary carbon atom substituents include hydrogen, halogen, —CN, —NO₂, —N₃, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR, —N(R^{Z})₂, or —SR, wherein R is hydrogen, halogen, —CN, —NO₂, —N₃, acyl, alkyl, alkenyl, alkynyl, carbocyclyl, aryl, heterocyclyl, or heteroaryl; R^{Z} is hydrogen, halogen, —CN, —NO₂, —N₃, acyl, alkyl, alkenyl, alkynyl, carbocyclyl, aryl, heterocyclyl, heteroaryl, or nitrogen protecting group. Exemplary carbon atom substituents include hydrogen, halogen, —CN, —NO₂, —N₃, acyl, alkyl, alkenyl, alkynyl, carbocyclyl, aryl, heterocyclyl, heteroaryl, —OR, —N(R^{Z})₂, or —SR, wherein R is hydrogen, halogen, —CN, —NO₂, —N₃, acyl, alkyl, alkenyl, alkynyl, carbocyclyl, aryl, heterocyclyl, or heteroaryl; R^{Z} is hydrogen, halogen, —CN, —NO₂, —N₃, acyl, alkyl, alkenyl, alkynyl, carbocyclyl, aryl, heterocyclyl, heteroaryl, or nitrogen protecting group.

As generally described above, in certain embodiments, a carbon atom is independently unsubstituted or substituted with hydrogen, halogen, —CN, —NO₂, —N₃, acyl, alkyl, alkenyl, alkynyl, carbocyclyl, aryl, heterocyclyl, heteroaryl, —OR, —N(R^{Z}), or —SR; a nitrogen atom is independently unsubstituted or substituted with hydrogen, acyl, alkyl, alkenyl, alkynyl, carbocyclyl, aryl, heterocyclyl, heteroaryl, or a nitrogen protecting group; an oxygen atom is independently unsubstituted or substituted with hydrogen, acyl, alkyl, alkenyl, alkynyl, carbocyclyl, aryl, heterocyclyl, heteroaryl, or oxygen protecting group; a sulfur atom is independently unsubstituted or substituted with hydrogen, acyl, alkyl, alkenyl, alkynyl, carbocyclyl, aryl, heterocyclyl, heteroaryl, or sulfur protecting group when attached to an sulfur atom; R is hydrogen, halogen, —CN, —NO₂, —N₃, acyl, alkyl, alkenyl, alkynyl, carbocyclyl, aryl, heterocyclyl, or heteroaryl; and

R^{Z} is hydrogen, halogen, —CN, —NO₂, —N₃, acyl, alkyl, alkenyl, alkynyl, carbocyclyl, aryl, heterocyclyl, heteroaryl, or nitrogen protecting group. “Halo” or “halogen” refers to fluorine (fluoro, —F), chlorine (chloro, —Cl), bromine (bromo, —Br), or iodine (iodo, —I).

“Acyl” as used herein refers to a moiety selected from the group consisting of —C(═O)R^{aa}, —CHO, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —C(═O)NR^{bb}SO₂R^{aa}, —C(═S)N(R^{bb})₂, —C(═O)SR^{aa}, and —C(═S)SR^{aa}, wherein R^{aa} and R^{bb} are as defined herein.

Nitrogen atoms can be substituted or unsubstituted as valency permits, and include primary, secondary, tertiary, and quarternary nitrogen atoms. Exemplary nitrogen atom substituents include, but are not limited to, hydrogen, —OH, —O⁻, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc}, and R^{dd} are as defined above. Exemplary nitrogen atom substituents include hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, or a nitrogen protecting group. Exemplary nitrogen atom substituents include hydrogen, acyl, alkyl, alkenyl, alkynyl, carbocyclyl, aryl, heterocyclyl, heteroaryl, or a nitrogen protecting group.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R_{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein. Exemplary oxygen atom substituents include hydrogen, acyl, alkyl, alkenyl, alkynyl, carbocyclyl, aryl, heterocyclyl, heteroaryl, or oxygen protecting group.

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is hydrogen, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, or sulfur protecting group. In certain embodiments, the substituent present on a sulfur atom is hydrogen, acyl, alkyl, alkenyl, alkynyl, carbocyclyl, aryl, heterocyclyl, heteroaryl, or sulfur protecting group.

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R_{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein; wherein X⁻ is a counterion. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB_{II}Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

As used herein, a “leaving group” (LG) is an art-understood term referring to a molecular fragment that departs with a pair of electrons in heterolytic bond cleavage, wherein the molecular fragment is an anion or neutral molecule. As used herein, a leaving group can be an atom or a group capable of being displaced by a nucleophile. See, for example, Smith, March Advanced Organic Chemistry 6th ed. (501-502). Exemplary leaving groups include, but are not limited to, halo (e.g., chloro, bromo, iodo) and activated substituted hydroxyl groups (e.g., —OC(═O)SR^{aa}, —OC(═O)R^{aa}, —OCO₂R^{aa}, —OC(O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). In some cases, the leaving group is a sulfonic acid ester, such as toluenesulfonate (tosylate, -OTs), methanesulfonate (mesylate, -OMs), p-bromobenzenesulfonyloxy (brosylate, -OBs), or trifluoromethanesulfonate (triflate, -OTf). In some cases, the leaving group is a brosylate, such as p-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

These and other exemplary substituents are described in more detail in the Detailed Description, Figures, Examples, and Claims. The invention is not intended to be limited in any manner by the above exemplary listing of substituents.

### Other Definitions

The following definitions are more general terms used throughout the present application:

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, non-toxic ammonium, quaternary ammonium, and amine cations formed using counterions such as halide, hydroxide, carboxylate, sulfate, phosphate, nitrate, loweralkyl sulfonate, and aryl sulfonate.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I′), Formula (IA), or Formulae (I)-(VII) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R.x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R.0.5H₂O)), and polyhydrates (x is a number greater than 1, e.g., dihydrates (R.2H₂O) and hexahydrates (R.6H₂O)).

As used herein, the term “tautomer” includes two or more interconvertible forms resulting from at least one formal migration of a hydrogen atom and at least one change in valency (e.g., a single bond to a double bond, a triple bond to a double bond, or vice versa). The exact ratio of the tautomers depends on several factors, including temperature, solvent, and pH. Tautomerizations (i.e., the reaction providing a tautomeric pair) may be catalyzed by acid or base. Exemplary tautomerizations include keto-to-enol; amide-to-imide; lactam-to-lactim; enamine-to-imine; and enamine-to-(a different) enamine tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorphs” refers to a crystalline form of a compound (or a salt, hydrate, or solvate thereof) in a particular crystal packing arrangement. All polymorphs have the same elemental composition. Different crystalline forms usually have different X-ray diffraction patterns, infrared spectra, melting points, density, hardness, crystal shape, optical and electrical properties, stability, and/or solubility. Recrystallization solvent, rate of crystallization, storage temperature, and other factors may cause one crystal form to dominate. Various polymorphs of a compound can be prepared by crystallization under different conditions.

The term “prodrugs” refer to compounds, including derivatives of the compounds of Formula (I′), Formula (IA), or Formulae (I)-(VII), which have cleavable groups and become by solvolysis or under physiological conditions the compounds of Formula (I′), Formula (IA), or Formulae (I)-(VII) which are pharmaceutically active in vivo. Such examples include, but are not limited to, choline ester derivatives and the like, N-alkylmorpholine esters and the like. Other derivatives of the compounds of this invention have activity in both their acid and acid derivative forms, but in the acid sensitive form often offers advantages of solubility, tissue compatibility, or delayed release in the mammalian organism (see, Bundgard, Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds of this invention are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds of Formula (I′), Formula (IA), or Formula (I)-(VII) may be preferred in certain instances.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

As used herein, the terms “condition,” “disease,” and “disorder” are used interchangeably.

An “effective amount” of a compound of Formula (I′), Formula (IA) or Formulae (I)-(VII) refers to an amount sufficient to elicit a desired biological response, i.e., treating the condition. As will be appreciated by those of ordinary skill in this art, the effective amount of a compound of Formula (I′), Formula (IA), or Formulae (I)-(VII) may vary depending on such factors as the desired biological endpoint, the pharmacokinetics of the compound, the condition being treated, the mode of administration, and the age and health of the subject. An effective amount encompasses therapeutic and prophylactic treatment.

A “therapeutically effective amount” of a compound of Formula (I′), Formula (IA), or Formulae (I)-(VII) is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

A “prophylactically effective amount” of a compound of Formula (I′), Formula (IA), Formulae (I)-(VII) is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

In certain embodiments, a compound of the present invention is provided as a salt. Salts are well known in the art. For example, Berge et al., describe pharmaceutically acceptable salts in detail in J. Pharmaceutical Sciences, 1977, 66, 1-19, incorporated herein by reference. Salts of the compounds of this invention include those derived from suitable inorganic and organic acids and bases. Examples include salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further salts include, when appropriate, ammonium, quaternary ammonium, and amine cations formed using counterions such as halide, hydroxide, carboxylate, sulfate, phosphate, nitrate, loweralkyl sulfonate and aryl sulfonate.

As used herein “inhibition,” “inhibiting,” and “inhibit”, refer to the ability of a compound to reduce, slow, halt or prevent activity of a particular biological process in a cell relative to vehicle. In certain embodiments, the biological process is in vitro (e.g., cellular assay). In certain embodiments, the biological process is in vivo. In certain embodiments, a probe compound of the present invention inhibits a glycosyltransferase protein.

As used herein, the term “effective amount” refers to the amount of a substance, compound, molecule, agent or composition that elicits the relevant response in vitro or in vivo. For example, in the case of a probe compound of the present invention used in an assay of the present invention, an effective amount of probe compound is an amount of probe compound that elicits the desired response, e.g., binding to a desired protein.

The term “independently” is used herein to indicate that the groups can be identical or different.

The terms “labeled”, “labeled with a detectable agent”, and “labeled with a detectable moiety” are used herein interchangeably. “Label” and “detectable moiety” are also used interchangeably herein. When used in reference to a probe compound, these terms specify that the probe compound can be detected or visualized. In certain embodiments, a label is selected such that it generates a signal which can be measured and whose intensity is related to the amount of probe compound bound to a protein (e.g., in a sample). A label may be directly detectable (i.e., it does not require any further reaction or manipulation to be detectable, e.g., a fluorophore is directly detectable) or it may be indirectly detectable (i.e., it is made detectable through reaction or binding with another entity that is detectable, e.g., a hapten is detectable by immunostaining after reaction with an appropriate antibody comprising a reporter such as a fluorophore). Labels suitable for use in the present invention may be detectable by any of a variety of means including, but not limited to, spectroscopic, photochemical, biochemical, immunochemical, electrical, optical or chemical means. Suitable labels include, but are not limited to, various ligands, radionuclides, fluorescent dyes, chemiluminescent agents, microparticles, enzymes, calorimetric labels, magnetic labels, and haptens.

The term “Staphylococcus species” refers to Gram-positive bacteria, which appear as grape-like clusters when viewed through a microscope and as large, round, golden-yellow colonies, often with .beta.-hemolysis, when grown on blood agar plates. An exemplary species of Staphylococcus is Staphylococcus aureus.

The term “Streptococcus species” refers to a genus of spherical, Gram-positive bacteria, and a member of the phylum Firmicutes. Streptococci are lactic acid bacteria. Streptococcus species include S. hemolyticus, S. mitis, S. salivarius, S. pneumoniae. Streptococcus species are responsible for infectious diseases such as meningitis, bacterial pneumonia, endocarditis, erysipelas, and necrotizing fasciitis (‘flesh-eating’ microbial infections).

The term “Enterococcus species” refers to a genus of lactic acid bacteria of the phylum Firmicutes. They are Gram-positive cocci which often occur in pairs (Diplococci, for example, Diplococcus pneumoniae). Enterococci are facultative anaerobic organisms.

The term “Bacillus species” refers to a large number of diverse, rod-shaped Gram-positive bacteria that are motile by peritrichous flagella and are aerobic, such as B. anthracis and B. subtilis or anaerobic such as Clostridium spp., for example, C. difficile. These bacilli belong to division Firmicutes.

The term “Mycobacterium species” refers to Gram-positive, non-motile, pleomorphic rods related to the actinomyces. Tuberculosis in humans is caused by Mycobacterium tuberculosis. MDR-TB (multi-drug resistant tuberculosis) describes strains of tuberculosis that are resistant to at least the two first-line TB drugs, isoniazid and rifampicin.

As used herein, the term “glycosyltransferase” refers to an enzyme that catalyzes the transfer of a monosaccharide unit from an activated sugar (glycosyl donor) to a glycosyl acceptor molecule. In certain embodiments, a glycosyltransferase described herein is a peptidoglycan glycosyltransferase.

As used herein, the term “infectious disease” refers to an illness caused by a pathogenic biological agent that results from transmission from an infected person, animal, or reservoir to a susceptible host, either directly or indirectly, through an intermediate plant or animal host, vector, or inanimate environment. Last J M. ed. A dictionary of epidemiology. 4th ed. New York: Oxford University Press, 1988. Infectious disease is also known as transmissible diseases or communicable diseases. In certain embodiments, infectious diseases may be asymptomatic for much or even all of their course in a given host. Infectious pathogens include some viruses, bacteria, fungi, protozoa, multicellular parasites, and aberrant proteins known as prions.

### BRIEF DESCRIPTION OF THE DRAWINGS

FIGS. 1A to 1C show the results of luminescence stress response assay of antibiotics (moenomycin A, penicillin G (PenG), and kanamycin (Kan)), compounds 1882L04, 593K11 and 1661H15 against S. aureus RN4220. The S. aureus RN4220 strain harboring pXEN plasmids with a P_{CwrA}-lux construct, termed P1-reporter strain, was grown up at 37° C. in chloramphenicol-complemented (10 μg/ml) TSB medium for 16-18 hours and diluted to OD₆₀₀=0.1. 150 μL of this culture were added to each well of a sterile, black and optically clear bottom 96-well plate (PerkinElmer). The bacterial cultures were incubated in a 37° C. shaker for 30 min before addition of test compounds. 1.5 μL of a DMSO-solution of the test compound was added per well, and the plate was incubated at 37° C. in a shaker. The OD₆₀₀ and luminescence signals of the P1-reporter strain were monitored using a Promega microplate reader every hour. The normalized luminescence (raw luminescence readings divided by OD₆₀₀) data was obtained and plotted as a function of time (Balibar et al., Microbiol. 2010, 156, 1372). FIG. 1A shows thenormalized luminescence obtained using the antibiotics moenomycin A (MmA, 0.009 μg/mL) and penicillin G (PenG, 1.25 μg/mL) as positive control. Kanamycin (Kan, 5 μg/mL) was used as negative control. FIG. 1B shows thenormalized luminescence obtained using compounds 593K11, 161H15, and 1882L04. FIG. 1C shows the chemical structures of compounds 593K11, 1661H15, and 1882L04.

FIGS. 2A and 2B show the results of luminescence stress response assay of exemplified compounds. The P1-reporter strain, was grown up at 37° C. in chloramphenicol-complemented (10 μg/ml) TSB medium for 16-18 hours and diluted to OD₆₀₀=0.1. 150 μL of this culture were added to each well of a sterile, black and optically clear bottom 96-well plate (PerkinElmer). The bacterial cultures were incubated in a 37° C. shaker for 30 min before addition of test compounds. 1.5 μL of a DMSO-solution of the test compound was added per well, and the plate was incubated at 37° C. in a shaker. The OD₆₀₀ and luminescence signals of the P1-reporter strain were monitored using a Promega microplate reader every hour. FIGS. 2A and 2B show normalized luminescence (raw luminescence readings divided by OD₆₀₀) obtained after 4 h for cultures containing different concentrations of test compound.

FIG. 2C shows the chemical structures of exemplified compounds in FIGS. 2A and 2B.

FIG. 2D shows compound 1882L04 displacement of probe CMG121 from S. aureus SgtB.

FIG. 3A shows Michaelis-Menten kinetics determination of uninhibited polymerization of Lipid II by S. aureus SgtB.

FIG. 3B shows Michaelis-Menten kinetics determination for polymerization of LpII by SgtB in the presence of compound 1882L04.

FIG. 4A shows moenomycin A bound in PGT domain of S. aureus PBP2 (Y196D). Isogenic MSSA strains can be used as a probe for SgtB activity in a cell. A moenomycin resistant MSSA strain (NE1) with a mutation located in the catalytic cleft of the PGT domain of S. aureus PBP2 (Y196D) has been identified. This mutation was found to interfere with moenomycin binding.

FIG. 4B shows western blot of S. aureus PBP2 (Y196D) and PBP2. PBP2 Y196D produces shorter polysaccharide chains than PBP2.

FIG. 4C shows western blot of S. aureus PBP2 (Y196D) followed by SgtB treatment. SgtB can elongate the short polymers produced by PBP2 Y196D. MSSA NE1 is sensitized to compounds that inhibit SgtB.

FIG. 5 shows MIC data of moenomycin, compounds 593K11, 1661H15, and 1882L04 against MSSA NEI. MSSA NEI is found sensitized to compound 1882L04.

FIG. 6 shows TEM imaging of S. aureus NEI untreated with any exemplified compounds.

FIGS. 7A and 7B show TEM imaging of S. aureus NEI treated with compound 1882L04. Cells are shown to have growth defects, indicating that compound 1882L04 interferes with the cell growth.

FIG. 8 shows growth of S. aureus RN4220 in the presence of compound 1882L04. The compound shows bacteriostatic activity (1-6 h) as evidenced by a steady number of CFUs.

FIG. 9A shows in vitro inhibition of S. aureus SgtB in the presence of compound 1882L04. Compound 1882L04 was found to have an IC₅₀ of 97 μM.

FIG. 9B shows in vitro inhibition of E. faecalis PBP2a in the presence of compound 1882L04. Compound 1882L04 was found to have an IC₅₀ of 337 μM.

FIGS. 10A to 10E show dose-dependent displacement of probe CMG121 from S. aureus SgtB with exemplified compounds determined by fluorescence polarization. FIG. 10F shows the IC₅₀ values of these exemplified compounds for displacing probe CMG121 from S. aureus SgtB.

FIG. 11A shows a MIC test of compound 1882L04 in MSSA Newman.

FIG. 11B shows a MIC test of compound 1882L04 in MSSA NE1.

FIG. 12 shows wells of MIC test of compound 1882L04 in MSSA Newman and MSSA NE1, respectively. The clear well with the lowest concentration of compound indicates the MIC. For MSSA Newman: 125 μg/mL; for MSSA NE1: 8 μg/mL.

FIG. 13 shows resistant mutant strains against compound 1882L04. A liquid culture of wild-type S. aureus RN4220 was grown up in the presence of 1882L04 (65 ug/mL) for 22 h. This culture was normalized to OD600=0.6, diluted 100×, and grown up in the presence of 1882L04 (65 ug/mL) and DMSO. The cultures obtained after 22 h were treated as mentioned before and grown up again. The fact that the cultures obtained after treatment with 1882L04 show the same growth behavior as wild-type S. aureus RN4220 in the presence of DMSO, but not as wild type S. aureus RN4220 in the presence of 1882L04, indicates, that the strains became resistant to 1882L04.

### DETAILED DESCRIPTION OF CERTAIN EMBODIMENTS

The present invention provides compounds of Formula (I′), Formula (IA), Formulae (I)-(VII), and pharmaceutically acceptable salts, solvates, hydrates, polymorphs, co-crystals, tautomers, stereoisomers, isotopically labeled derivatives, and prodrugs thereof. These compounds have been found to be peptidoglycan glycosyltransferases (PGT) inhibitors. Also provided are methods of using these peptidoglycan glycosyltransferases inhibitors, such as compounds of Formula (I′), Formula (IA), Formulae (I)-(VII), or pharmaceutically acceptable salts, solvates, hydrates, polymorphs, co-crystals, tautomers, stereoisomers, isotopically labeled derivatives, and prodrugs thereof, to inhibit the activities of peptidoglycan glycosyltransferases in a subject or biological sample. The present invention further provides methods of using compounds of Formula (I′), Formula (IA), Formulae (I)-(VII), or pharmaceutically acceptable salts, solvates, hydrates, polymorphs, co-crystals, tautomers, stereoisomers, isotopically labeled derivatives, and prodrugs thereof as therapeutics, e.g., in the treatment and/or prevention of microbial infections (e.g., bacterial infections). In certain embodiments, the bacterium is a Gram-positive bacterium. Exemplary Gram-positive bacterium for which the inventive compounds may be used to treat or prevent an infection include, but are not limited to, Staphylococcus, Streptococcus, Micrococcus, Peptococcus, Peptostreptococcus, Enterococcus, Bacillus, Clostridium, Lactobacillus, Listeria, Erysipelothrix, Propionibacterium, Eubacterium, Corynebacterium, Capnocytophaga, Bifidobacterium, and Gardnerella. In certain embodiments, the bacterium is a Gram-negative bacterium. Exemplary Gram-negative bacterium for which the inventive compounds may be used to treat or prevent an infection include, but are not limited to, Escherichia, Citrobacter, Enterobacter, Klebsiella, Proteus, Serratia, Shigella, Salmonella, Morganella, Providencia, Edwardsiella, Erwinia, Hafnia, Yersinia, Acinetobacter, Vibrio, Aeromonas, Pseudomonas, Haemophilus, Pasteurella, Campylobacter, Helicobacter, Branhamella, Moraxella, Neisseria, Veillonella, Fusobacterium, Bacteroides, Actinobacillus, Aggregatibacter, Agrobacterium, Porphyromonas, Prevotella, Ruminobacter, Roseburia, Caulobacter, Francisella, Borrelia, Treponema, Brucella, and Rickettsia. In certain embodiments, the Gram-negative bacterium is selected from the group consisting of Escherichia coli, Morganella morganii, Branhamella catarrhalis, Veillonella parvula, Actinobacillus actinomycetemcomitans, Aggregatibacter actinomycetemcomitans, Caulobacter crescentus, and Treponema pallidum. In certain embodiments, the bacterium is a drug-resistant bacterium. In certain embodiments, the bacterium is methicillin-resistant. In certain embodiments, the bacterium is vancomycin-resistant. Exemplary bacterial strains for which the inventive compounds may be used to treat or prevent an infection include, but are not limited to, Staphylococcus saprophyticus, Staphylococcus xylosus, Staphylococcus lugdunensis, Staphylococcus schleiferi, Staphylococcus caprae, Staphylococcus epidermidis, Staphylococcus saprophyticus, Staphylococcus warneri, Staphylococcus aureus, Staphylococcus hominis, Enterococcusfaecalis, Proprionibacterium acnes, Bacillus cereus, Bacillus subtilis, Listeria monocytogenes, Streptococcus pyrogenes, Streptococcus salivariu, Streptococcus mutans and Streptococcus pneumonia, methicillin-resistant Staphylococcus aureus (MRSA), methicillin-resistant Staphylococcus epidermidis (MRSE), penicillin-resistant Streptococcus pneumonia, quinolone-resistant Staphylococcus aureus (QRSA), vancomycin-resistant Staphylococcus aureus (VRSA), vancomycin-resistant Enterococci (VRE), and multi-drug resistant Mycobacterium tuberculosis. In certain embodiments, the drug-resistant bacterium is selected from the group consisting of methicillin-resistant Staphylococcus aureus (MRSA), methicillin-resistant Staphylococcus epidermidis (MRSE), penicillin-resistant Streptococcus pneumonia, quinolone-resistant Staphylococcus aureus (QRSA), vancomycin-resistant Staphylococcus aureus (VRSA), vancomycin-resistant Enterococci (VRE), and multi-drug resistant Mycobacterium tuberculosis.

### Compounds

As generally described above, provided herein are compounds of Formula (I′):

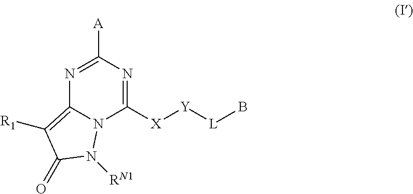

and pharmaceutically acceptable salts, solvates, hydrates, polymorphs, co-crystals, tautomers, stereoisomers, isotopically labeled derivatives, and prodrugs thereof,
wherein

A is independently optionally substituted C₂₋₆ alkyl, optionally substituted aryl, optionally substituted carbocyclyl, optionally substituted 5-membered heteroaryl, or optionally substituted 6-membered heteroaryl;

B is independently hydrogen, optionally substituted alkyl, optionally substituted alkenyl, optionally substituted alkynyl, optionally substituted aryl, optionally substituted carbocyclyl, optionally substituted heterocyclyl, or optionally substituted heteroaryl;

Y is a bond, optionally substituted C₁₋₆ alkylene, optionally substituted C₃₋₆ carbocyclylene, or optionally substituted heterocyclylene;

X is a bond, —O—, —CH₂—, —NR^{NX}—, —NR^{NX}—C(═O)—NR^{NX}—, or optionally substituted heterocyclylene;

L is a bond, —O—, —C(═O)—, —NR^{LB}C(═O)—, —C(═O)NR^{LB}—, —NR^{LB}—, or —SO₂—;

each instance of R^{LB} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or R^{LB} and B are taken together with their intervening atoms to form an optionally substituted heterocyclic ring;

R₁ is hydrogen, halogen, or optionally substituted C₁₋₆ alkyl;

R^{N1} is hydrogen, optionally substituted C₁₋₆ alkyl, or a nitrogen protecting group; and

R^{NX} is hydrogen, optionally substituted C₁₋₆ alkyl, or a nitrogen protecting group;

provided that the compound of Formula (I′) is not of the formula:

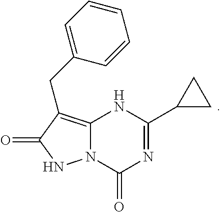 [SMILES: O=c1[nH]n2c(=O)nc(C3CC3)[nH]c2c1Cc1ccccc1]

As generally described above, provided herein are compounds of Formula (I):

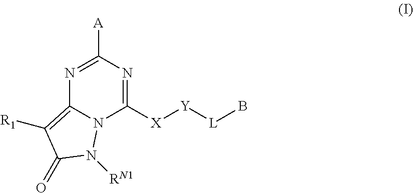

and pharmaceutically acceptable salts, solvates, hydrates, polymorphs, co-crystals, tautomers, stereoisomers, isotopically labeled derivatives, and prodrugs thereof,
wherein

A is independently optionally substituted aryl, optionally substituted 5-membered heteroaryl, or optionally substituted 6-membered heteroaryl;

B is independently hydrogen, optionally substituted alkyl, optionally substituted alkenyl, optionally substituted alkynyl, optionally substituted aryl, optionally substituted carbocyclyl, optionally substituted heterocyclyl, or optionally substituted heteroaryl;

Y is a bond, optionally substituted C₁₋₆ alkylene, optionally substituted C₃₋₆ carbocyclylene, or optionally substituted heterocyclylene;

X is a bond, —O—, —S—, —CH₂—, —NR^{NX}—, —NR^{NX}C(═O)—NR^{NX}—, or optionally substituted heterocyclylene;

L is a bond, —O—, —C(═O)—, —NR^{LB}C(═O)—, —C(═O)NR^{LB}—, —NR^{LB}—, or —SO₂—;

each instance of R^{LB} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or R^{LB} and B are taken together with their intervening atoms to form an optionally substituted heterocyclic ring;

R₁ is hydrogen, halogen, or optionally substituted C₁₋₆ alkyl;

R^{N1} is hydrogen, optionally substituted C₁₋₆ alkyl, or a nitrogen protecting group; and

R^{NX} is hydrogen, optionally substituted C₁₋₆ alkyl, or a nitrogen protecting group.

As generally described above, provided herein are compounds of Formula (IA):

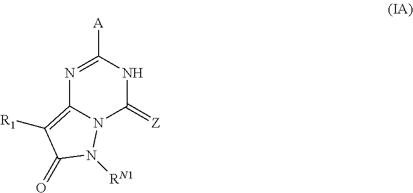

and pharmaceutically acceptable salts, solvates, hydrates, polymorphs, co-crystals, tautomers, stereoisomers, isotopically labeled derivatives, and prodrugs thereof, wherein

A is independently optionally substituted C₂₋₆ alkyl, optionally substituted aryl, optionally substituted C₄₋₁₀ carbocyclyl, optionally substituted 5-membered heteroaryl, or optionally substituted 6-membered heteroaryl;

Z is S or O;

R₁ is hydrogen, halogen, or optionally substituted C₁₋₆ alkyl;

R^{N1} is hydrogen, optionally substituted C₁₋₆ alkyl, or a nitrogen protecting group; and

R^{NX} is hydrogen, optionally substituted C₁₋₆ alkyl, or a nitrogen protecting group;

provided that the compound of Formula (IA) is not of the formula:

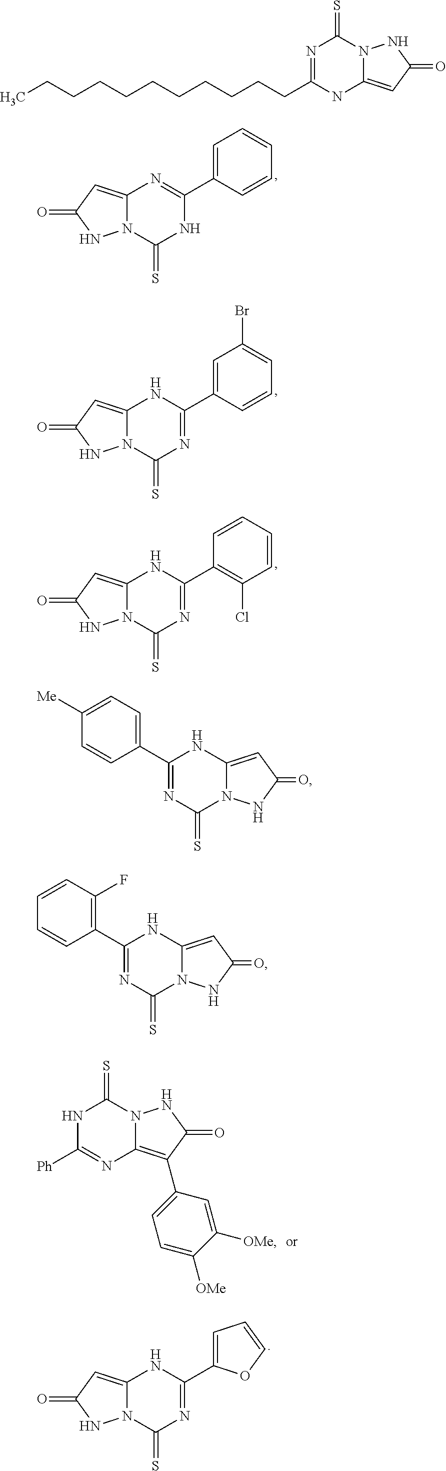

As generally defined herein, as applicable to Formula (I′), (IA), (I), (II), (IV), (VI), and (VII), R₁ is hydrogen, halogen, or optionally substituted C₁₋₆ alkyl. In some embodiments, R₁ is hydrogen. In some embodiments, R₁ is halogen. In certain embodiments, R₁ is F. In certain embodiments, R₁ is Cl. In certain embodiments, R₁ is Br. In certain embodiments, R₁ is I. In certain embodiments, R₁ is optionally substituted C₁₋₆ alkyl. In certain embodiments, R₁ is optionally substituted methyl. In certain embodiments, R₁ is methyl. In certain embodiments, R₁ is ethyl. In certain embodiments, R₁ is of the formula: —CH₂(R^{X}), wherein R^{X} is carbocyclyl, aryl, or heteroaryl. In certain embodiments, R₁ is of the formula: —CH₂(carbocyclyl). In certain embodiments, R₁ is of the formula:

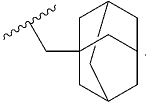 [SMILES: *CC12CC3CC(CC(C3)C1)C2]

In certain embodiments, R₁ is of the formula: —CH₂(aryl). In certain embodiments, R₁ is optionally substituted benzyl. In certain embodiments, R₁ is of the formula:

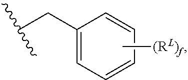

wherein each instance of R^{L} is independently hydrogen, halogen, —CN, —NO₂, —N₃, acyl, optionally substituted alkyl, alkenyl, alkynyl, carbocyclyl, aryl, heterocyclyl, or heteroaryl; and f is 1, 2, 3, 4, or 5. In certain embodiments, f is 1. In certain embodiments, f is 2. In certain embodiments, f is 3. In certain embodiments, f is 4. In certain embodiments, f is 5. In certain embodiments, R₁ is benzyl. In certain embodiments, R₁ is of the formula: —CH₂(heteroaryl). In certain embodiments, R₁ is of the formula:

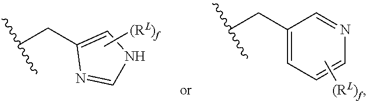

wherein each instance of R^{L} is independently hydrogen, halogen, —CN, —NO₂, —N₃, acyl, alkyl, alkenyl, alkynyl, carbocyclyl, aryl, heterocyclyl, or heteroaryl; and f is 1, 2, 3, 4, or 5). In certain embodiments, R₁ is of the formula:

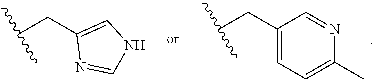

As generally defined herein, as applicable to all Formulae, R^{N1} is hydrogen, optionally substituted C₁₋₆ alkyl, or a nitrogen protecting group. In some embodiments, R^{N1} is hydrogen. In some embodiments, R^{N1} is optionally substituted C₁₋₆ alkyl. In certain embodiments, R^{N1} is methyl. In certain embodiments, R^{N1} is ethyl. In certain embodiments, R^{N1} is propyl. In certain embodiments, R^{N1} is a nitrogen protecting group. In certain embodiments, R^{N1} is Bn, BOC, Cbz, Fmoc, trifluoroacetyl, triphenylmethyl, or Ts.

As generally described above, X is a bond, —O—, —S—, —CH₂—, —NR^{NX}—, —NR^{NX}C(═O)—NR^{NX}—, or optionally substituted heterocyclylene. In certain embodiments, X is a bond. In certain embodiments, X is —O—. In certain embodiments, X is —S—. In certain embodiments, X is —CH₂—. In certain embodiments, X is —NR^{NX}—, wherein R^{NX} is hydrogen, optionally substituted C₁₋₆ alkyl, or a nitrogen protecting group. In certain embodiments, X is —NH—. In certain embodiments, X is —NR^{NX}—, wherein R^{NX} is optionally substituted C₁₋₆ alkyl. In certain embodiments, X is —NR^{NX}—, wherein R^{NX} is unsubstituted C₁₋₆ alkyl. In certain embodiments, X is —NR^{NX}—, wherein R^{NX} is unsubstituted C₁₋₃ alkyl. In certain embodiments, X is —NR^{NX}—, wherein R^{NX} is methyl, ethyl, or propyl. In certain embodiments, X is

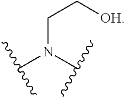 [SMILES: *N(*)CCO]

In certain embodiments, X is

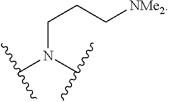 [SMILES: *N(*)CCCN(C)C]

In certain embodiments, X is —NR^{NX}—, wherein R^{NX} is a nitrogen protecting group. In certain embodiments, X is —NR^{NX}—, wherein R^{NX} is Bn, BOC, Cbz, Fmoc, trifluoroacetyl, triphenylmethyl, or Ts. In certain embodiments, X is —NR^{NX}—, wherein R^{NX} is acetyl (Ac). In certain embodiments, X is —NR^{NX}—C(═O)—NR^{NX}—, wherein each instance of R^{NX} is independently hydrogen, optionally substituted C₁₋₆ alkyl, or a nitrogen protecting group. In certain embodiments, X is —NH—C(═O)—NH—. In certain embodiments, X is optionally substituted heterocyclylene. In certain embodiments, X is optionally substituted 3- to 6-membered heterocyclylene. In certain embodiments, X is optionally substituted 3-membered heterocyclylene. In certain embodiments, X is optionally substituted 4-membered heterocyclylene. In certain embodiments, X is optionally substituted 5-membered heterocyclylene. In certain embodiments, X is optionally substituted 6-membered heterocyclylene.

In certain embodiments, X is of the formula:

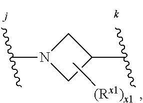

wherein

k indicates the point of attachment to Y;

j indicates the point of attachment to the triazine ring;

each instance of R^{x1} is independently selected from the group consisting of hydrogen, halogen, —CN, —NO₂, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, or —SO₂N(R^{B})₂;

each instance of R^{A} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, and an oxygen protecting group;

each instance of R^{B} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, and a nitrogen protecting group, or two R^{B} groups are taken together with their intervening atoms to form an optionally substituted heterocyclic ring; and

x1 is independently 0, 1, 2, 3, or 4.

In certain embodiments, R^{x1} is hydrogen. In certain embodiments, x1 is 0. In certain embodiments, x1 is 1.

In certain embodiments, X is of the formula:

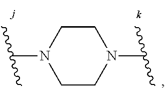 [SMILES: *N1CCN(*)CC1]

wherein

k indicates the point of attachment to Y; and

j indicates the point of attachment to the triazine ring.

In certain embodiments, X is of the formula:

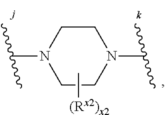

wherein

k indicates the point of attachment to Y;

j indicates the point of attachment to the triazine ring;

each instance of R^{x2} is independently selected from the group consisting of hydrogen, halogen, —CN, —NO₂, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, or —SO₂N(R^{B})₂;

each instance of R^{A} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, and an oxygen protecting group;

each instance of R^{B} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, and a nitrogen protecting group, or two R^{B} groups are taken together with their intervening atoms to form an optionally substituted heterocyclic ring; and

x2 is 0, or an integer between 1 and 8, inclusive.

In certain embodiments, R^{x2} is hydrogen. In certain embodiments, x2 is 0. In certain embodiments, x2 is 1. In certain embodiments, x2 is 2. In certain embodiments, x2 is 3. In certain embodiments, x2 is 4.

As generally described above, A is independently optionally substituted aryl, optionally substituted 5-membered heteroaryl, or optionally substituted 6-membered heteroaryl. In certain embodiments, A is optionally substituted C₂₋₆ alkyl. In certain embodiments, A is optionally substituted ethyl. In certain embodiments, A is optionally substituted propyl. In certain embodiments, A is optionally substituted butyl. In certain embodiments, A is of the formula:

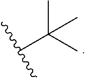 [SMILES: *C(C)(C)C]

In certain embodiments, A is optionally substituted carbocyclyl. In certain embodiments, A is optionally substituted C₄₋₁₀ carbocyclyl. In Formula (I′), or Formula (IA), in certain embodiments, A is optionally substituted C₄₋₁₀ carbocyclyl. In certain embodiments, A is of the formula:

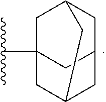 [SMILES: *C12CC3CC(CC(C3)C1)C2]

In certain embodiments, A is optionally substituted aryl. In certain embodiments, A is optionally substituted phenyl. In certain embodiments, A is unsubstituted phenyl. In certain embodiments, A is substituted phenyl. In certain embodiments, A is mono-substituted phenyl. In certain embodiments, A is di-substituted phenyl. In certain embodiments, A is tri-substituted phenyl. In certain embodiments, A is tetra-substituted or penta-substituted phenyl.

In certain embodiments, A is of formula:

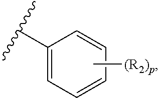

wherein p is 0, 1, 2, 3, 4, or 5; and each instance of R₂ is independently selected from the group consisting of hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(═O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, and —SO₂N(R^{B})₂. In certain embodiments, each instance of R₂ is independently selected from the group consisting of hydrogen, halogen, —CN, —NO₂, —N₃, substituted or unsubstituted acyl, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{A}, —N(R^{B})₂, and —SR^{A}. In certain embodiments, each instance of R₂ is independently selected from the group consisting of hydrogen, halogen, —CN, —NO₂, —N₃, acyl, alkyl, alkenyl, alkynyl, carbocyclyl, aryl, heterocyclyl, heteroaryl, —OR^{A}, —N(R^{B})₂, and —SR^{A}.

As generally defined herein, p is 0, 1, 2, 3, 4, or 5. In certain embodiments, R₂ is hydrogen, p is 5, and A is unsubstituted phenyl. In certain embodiments, p is 0, and A is unsubstituted phenyl. In certain embodiments, p is 1. In certain embodiments, A is of the formula:

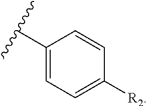

In certain embodiments, A is of the formula:

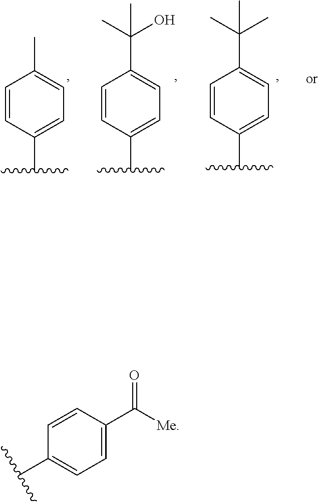

In certain embodiments, A is of the formula:

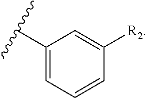

In certain embodiments, A is of the formula:

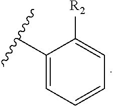

In certain embodiments, p is 2. In certain embodiments, A is of the formula:

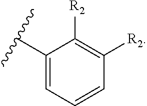

In certain embodiments, A is of the formula:

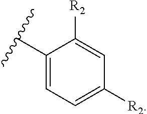

In certain embodiments, A is of the formula:

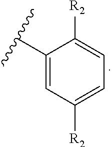

In certain embodiments, A is of the formula:

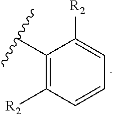

In certain embodiments, A is of the formula:

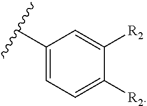

In certain embodiments, A is of the formula:

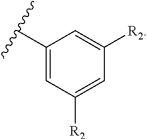

In certain embodiments, p is 3. In certain embodiments, A is of the formula:

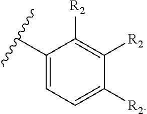

In certain embodiments, A is of the formula:

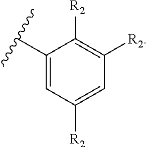

In certain embodiments, A is of the formula:

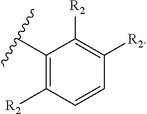

In certain embodiments, A is of the formula:

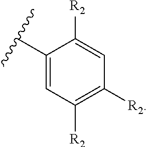

In certain embodiments, A is of the formula:

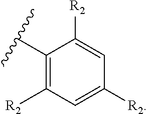

In certain embodiments, p is 4. In certain embodiments, A is of the formula:

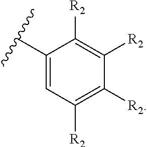

In certain embodiments, A is of the formula:

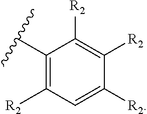

In certain embodiments, A is of the formula:

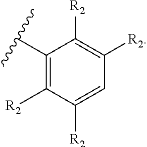

In certain embodiments, p is 5, and A is of the formula:

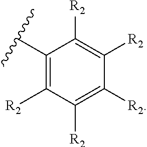

As generally defined herein, each instance of R₂ is independently selected from the group consisting of hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(═O)OR^{A}, —C(═O)SR^{A}, —C(═O)N(R^{B})₂, —C(═O)N(R^{B})N(R^{B})₂, —OC(O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, and —SO₂N(R^{B})₂. In certain embodiments, each instance of R₂ is independently selected from the group consisting of hydrogen, halogen, —CN, —NO₂, —N₃, substituted or unsubstituted alkyl, substituted or unsubstituted alkenyl, substituted or unsubstituted alkynyl, substituted or unsubstituted carbocyclyl, substituted or unsubstituted aryl, substituted or unsubstituted heterocyclyl, substituted or unsubstituted heteroaryl, —OR^{A}, —N(R^{B})₂, —SR^{A}, and —C(═O)R^{A}. In certain embodiments, each instance of R₂ is independently selected from the group consisting of hydrogen, halogen, —CN, —NO₂, —N₃, alkyl, alkenyl, alkynyl, carbocyclyl, aryl, heterocyclyl, heteroaryl, —OR^{A}, —N(R^{B})₂, —SR^{A}, and —C(═O)R^{A}. As generally defined herein, each instance of R^{A} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and an oxygen protecting group; and each instance of R^{B} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group, or two R^{B} groups are taken together with their intervening atoms to form an optionally substituted heterocyclic ring. In certain embodiments, each instance of R^{A} is independently selected from the group consisting of hydrogen, alkyl, alkenyl, alkynyl, carbocyclyl, heterocyclyl, aryl, heteroaryl, and an oxygen protecting group. In certain embodiments, each instance of R^{B} is independently selected from the group consisting of hydrogen, alkyl, alkenyl, alkynyl, carbocyclyl, heterocyclyl, aryl, heteroaryl, and an nitrogen protecting group. In certain embodiments, two R^{B} groups are taken together with their intervening atoms to form a heterocyclic ring (e.g., unsubstituted 5-to 10-membered monocyclic or bicyclic heterocyclic ring, wherein one or two atoms in the heterocyclic ring are independently nitrogen, oxygen, or sulfur).

In some embodiments, R₂ is halogen. In certain embodiments, R₂ is F. In certain embodiments, R₂ is Cl. In certain embodiments, R₂ is Br. In certain embodiments, R₂ is I. In some embodiments, R₂ is optionally substituted alkyl, optionally substituted alkenyl, optionally substituted alkynyl, or optionally substituted carbocyclyl. In certain embodiments, R₂ is optionally substituted C₁₋₆ alkyl, optionally substituted C₂₋₆ alkenyl, optionally substituted C₂₋₆ alkynyl, or optionally substituted C₃₋₆ carbocyclyl. In certain embodiments, R₂ is optionally substituted C₁₋₆ alkyl. In certain embodiments, R₂ is methyl. In certain embodiments, R₂ is ethyl. In certain embodiments, R₂ is propyl. In certain embodiments, R₂ is butyl. In certain embodiments, R₂ is pentyl. In certain embodiments, R₂ is isopropyl, isobutyl, or isoamyl. In certain embodiments, R₂ is of the formula:

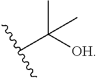 [SMILES: *C(C)(C)O]

In certain embodiments, R₂ is tert-butyl. In some embodiments, R₂ is —CN. In some embodiments, R₂ is —NO₂. In some embodiments, R₂ is —N₃. In some embodiments, R₂ is optionally substituted carbocyclyl, optionally substituted aryl, optionally substituted heterocyclyl, or optionally substituted heteroaryl. In some embodiments, R₂ is cyclopropyl or cyclobutyl. In some embodiments, R₂ is —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, or —SO₂N(R^{B})₂. In certain embodiments, R₂ is —N(R^{B})₂. In certain embodiments, R₂ is —NHR^{B}. In certain embodiments, R₂ is —NHR^{B}, wherein R^{B} is optionally substituted C₁₋₆ alkyl. In certain embodiment, R₂ is —NHR^{B}, wherein R^{B} is substituted C₁₋₆ alkyl. In certain embodiments, R₂ is —NHR^{B}, wherein R^{B} is unsubstituted C₁₋₆ alkyl. In certain embodiments, R₂ is —NHR^{B}, wherein R^{B} is methyl, ethyl, or propyl. In certain embodiments, R₂ is —N(R^{B})₂, wherein each R^{B} is independently optionally substituted C₁₋₆ alkyl. In certain embodiments, R₂ is —N(R^{B})₂, wherein each R^{B} is independently unsubstituted C₁₋₆ alkyl. In certain embodiments, R₂ is —N(R^{B})₂, wherein each R^{B} is independently selected from the group consisting of methyl, ethyl, isopropyl, isobutyl, isoamyl, and benzyl. In some embodiments, R₂ is —N(R^{B})₂, wherein each R^{B} is the same. In some embodiments, R₂ is —N(R^{B})₂, wherein each R^{B} is different. In certain embodiments, R₂ is —NH₂. In certain embodiments, R₂ is —OR^{A}. In certain embodiments, R₂ is —OH. In certain embodiments, R₂ is —OR^{A}, wherein R^{A} is optionally substituted alkyl, optionally substituted alkenyl, optionally substituted alkynyl, or optionally substituted carbocyclyl. In certain embodiments, R₂ is —OR^{A}, wherein R^{A} is optionally substituted C₁₋₆ alkyl. In certain embodiments, R₂ is —OR^{A}, wherein R^{A} is unsubstituted C₁₋₆ alkyl. In certain embodiments, R₂ is —O-methyl, —O-ethyl, —O-propyl, —O— isopropyl, —O-isobutyl, or —O-isoamyl. In certain embodiments, R₂ is —OR^{A}, wherein R^{A} is substituted C₁₋₆ alkyl. In certain embodiments, R₂ is —OR^{A}, wherein R^{A} is optionally substituted aryl. In certain embodiments, R₂ is —O-phenyl. In certain embodiments, R₂ is —OR^{A}, wherein R^{A} is optionally substituted heteroaryl.

In certain embodiments, p is 1, and R₂ is optionally substituted C₁₋₆ alkyl, optionally substituted C₂₋₆ alkenyl, or —OR^{A}. In certain embodiments, p is 1, and R₂ is optionally substituted C₁₋₆ alkyl, optionally substituted C₂₋₆ alkenyl, —OR^{A}, or —C(═O)R^{A}. In certain embodiments, p is 1, and R₂ is optionally substituted C₁₋₆ alkyl, optionally substituted C₂₋₆ alkenyl, or —OR^{A} at the ortho-position of the phenyl ring. In certain embodiments, p is 1, and R₂ is optionally substituted C₁₋₆ alkyl, optionally substituted C₂₋₆ alkenyl, or —OR^{A} at the meta-position of the phenyl ring. In certain embodiments, p is 1, and R₂ is optionally substituted C₁₋₆ alkyl, optionally substituted C₂₋₆ alkenyl, or —OR^{A} at the para-position of the phenyl ring. In certain embodiments, p is 1, and R₂ is optionally substituted C₁₋₆ alkyl, optionally substituted C₂₋₆ alkenyl, —OR^{A}, or —C(═O)R^{A} at the para-position of the phenyl ring. In certain embodiments, p is 1, and R₂ is optionally substituted C₁₋₆ alkyl, optionally substituted C₂₋₆ alkenyl, or —OR^{A}, wherein R^{A} is hydrogen, optionally substituted C₁₋₆ alkyl, or an oxygen protecting group. In certain embodiments, p is 1, and R₂ is methyl, ethyl, propyl, isopropyl, or tert-butyl. In certain embodiments, p is 1, and R₂ is allyl. In certain embodiments, p is 1; and R₂ is F, Cl, Br, or I. In certain embodiments, p is 1, and R₂ is —OH, methoxy, or ethoxy. In certain embodiments, p is 2, and each instance of R₂ is optionally substituted C₁₋₆ alkyl, optionally substituted C₂₋₆ alkenyl, or —OR^{A}. In certain embodiments, p is 2, and each instance of R₂ is independently optionally substituted C₁₋₆ alkyl, optionally substituted C₂₋₆ alkenyl, or —OR^{A}, wherein R^{A} is hydrogen, optionally substituted C₁₋₆ alkyl, or an oxygen protecting group. In certain embodiments, p is 2, and each instance of R₂ is independently methyl, ethyl, propyl, isopropyl, tert-butyl, allyl, —OH, methoxy, or ethoxy. In certain embodiments, p is 3, and each instance of R₂ is optionally substituted C₁₋₆ alkyl, optionally substituted C₂₋₆ alkenyl, or —OR^{A}. In certain embodiments, p is 3, and each instance of R₂ is independently optionally substituted C₁₋₆ alkyl, optionally substituted C₂₋₆ alkenyl, or —OR^{A}, wherein R^{A} is hydrogen, optionally substituted C₁₋₆ alkyl, or an oxygen protecting group. In certain embodiments, p is 3, and each instance of R₂ is independently methyl, ethyl, propyl, isopropyl, tert-butyl, allyl, —OH, methoxy, or ethoxy. In certain embodiments, p is 4, and each instance of R₂ is optionally substituted C₁₋₆ alkyl, optionally substituted C₂₋₆ alkenyl, or —OR^{A}. In certain embodiments, p is 4, and each instance of R₂ is independently optionally substituted C₁₋₆ alkyl, optionally substituted C₂₋₆ alkenyl, or —OR^{A}, wherein R^{A} is hydrogen, optionally substituted C₁₋₆ alkyl, or an oxygen protecting group. In certain embodiments, p is 4, and each instance of R₂ is independently methyl, ethyl, propyl, isopropyl, tert-butyl, allyl, —OH, methoxy, or ethoxy. In certain embodiments, p is 5, and each instance of R₂ is optionally substituted C₁₋₆ alkyl, optionally substituted C₂₋₆ alkenyl, or —OR^{A}. In certain embodiments, p is 5, and each instance of R₂ is independently optionally substituted C₁₋₆ alkyl, optionally substituted C₂₋₆ alkenyl, or —OR^{A}, wherein R^{A} is hydrogen, optionally substituted C₁₋₆ alkyl, or an oxygen protecting group. In certain embodiments, p is 5, and each instance of R₂ is independently methyl, ethyl, propyl, isopropyl, tert-butyl, allyl, —OH, methoxy, or ethoxy.

In certain embodiments, A is selected from the group consisting of:

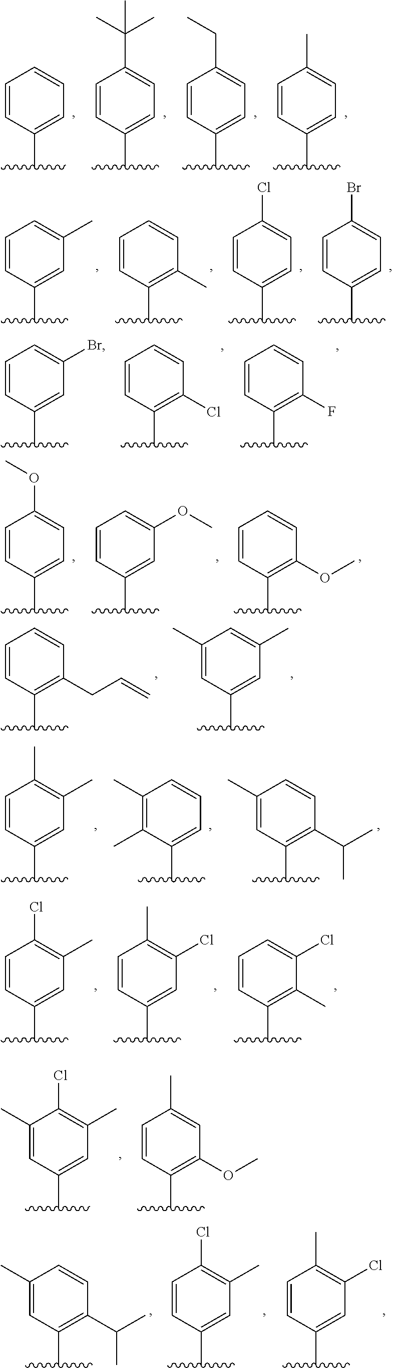

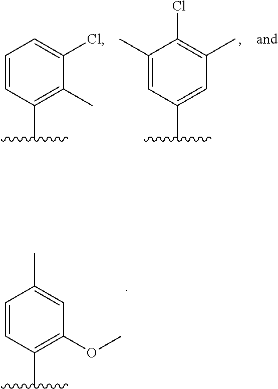

In certain embodiments, A is selected from the group consisting of:

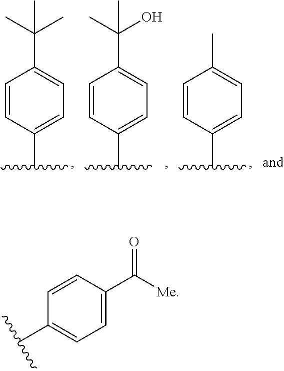

In certain embodiments, A is optionally substituted, 5-membered heteroaryl. In certain embodiments, A is substituted, 5-membered heteroaryl. In certain embodiments, A is unsubstituted, 5-membered heteroaryl. In certain embodiments, A is optionally substituted, 5-membered heteroaryl with one oxygen, nitrogen, or sulfur. In certain embodiments, A is of the formula:

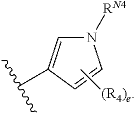

In certain embodiments, A is of the formula:

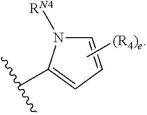

In certain embodiments, A is of the formula:

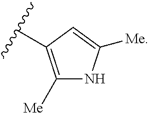 [SMILES: *c1cc(C)[nH]c1C]

In certain embodiments, A is of the formula:

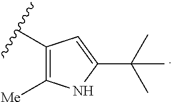 [SMILES: *c1cc(C(C)(C)C)[nH]c1C]

In certain embodiments, A is of the formula:

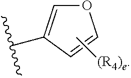

In certain embodiments, A is of the formula:

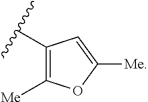 [SMILES: *c1cc(C)oc1C]

In certain embodiments, A is of the formula:

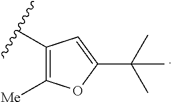 [SMILES: *c1cc(C(C)(C)C)oc1C]

In certain embodiments, A is of the formula:

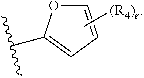

In certain embodiments, A is of the formula:

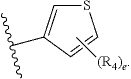

In certain embodiments, A is of the formula:

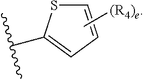

In certain embodiments, A is of the formula:

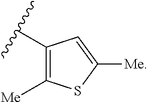 [SMILES: *c1cc(C)sc1C]

In certain embodiments, A is of the formula:

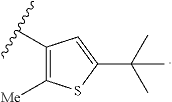 [SMILES: *c1cc(C(C)(C)C)sc1C]

In certain embodiments, A is optionally substituted, 5-membered heteroaryl with two heteroatoms each independently selected from the group consisting of S, N, and O. In certain embodiments, A is optionally substituted, 5-membered heteroaryl with three heteroatoms each independently selected from the group consisting of S, N, and O. In certain embodiments, A is optionally substituted, 5-membered heteroaryl with four heteroatoms each independently selected from the group consisting of S, N, and O.

In certain embodiments, A is optionally substituted, 6-membered heteroaryl. In certain embodiments, A is substituted, 6-membered heteroaryl. In certain embodiments, A is unsubstituted, 6-membered heteroaryl. In certain embodiments, A is optionally substituted 6-membered, heteroaryl with one oxygen, nitrogen, or sulfur. In certain embodiments, A is of formula:

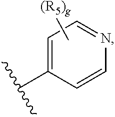

wherein each instance of R₅ is independently selected from the group consisting of hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, and —C(═O)R^{A}, and g is independently 0, 1, 2, 3, 4, or 5. In certain embodiments, g is 0. In certain embodiments, g is 1. In certain embodiments, g is 2. In certain embodiments, g is 3. In certain embodiments, g is 4. In certain embodiments, g is 5. In certain embodiments, R₅ is hydrogen. In some embodiments, R₅ is halogen. In certain embodiments, R₅ is F. In certain embodiments, R₅ is Cl. In certain embodiments, R₅ is Br. In certain embodiments, R₅ is I. In some embodiments, R₅ is optionally substituted alkyl, optionally substituted alkenyl, optionally substituted alkynyl, or optionally substituted carbocyclyl. In certain embodiments, R₅ is optionally substituted C₁₋₆ alkyl, optionally substituted C₂₋₆ alkenyl, optionally substituted C₂₋₆ alkynyl, or optionally substituted C₃₋₆ carbocyclyl. In certain embodiments, R₅ is optionally substituted C₁₋₆ alkyl. In certain embodiments, R₃ is methyl. In certain embodiments, R₅ is ethyl. In certain embodiments, R₅ is propyl. In certain embodiments, R₅ is butyl. In certain embodiments, R₅ is pentyl. In certain embodiments, R₅ is isopropyl, isobutyl, or isoamyl. In certain embodiments, R₅ is tert-butyl. In some embodiments, R₅ is —CN. In some embodiments, R₅ is —NO₂. In some embodiments, R₅ is —N₃. In some embodiments, R₅ is optionally substituted carbocyclyl, optionally substituted aryl, optionally substituted heterocyclyl, or optionally substituted heteroaryl. In some embodiments, R₅ is cyclopropyl or cyclobutyl. In certain embodiments, A is

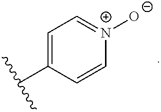 [SMILES: *c1cc[n+]([O-])cc1]

In certain embodiments, A is optionally substituted, 6-membered heteroaryl with two heteroatoms each independently selected from the group consisting of S, N, and O. In certain embodiments, A is optionally substituted, 6-membered heteroaryl with three heteroatoms each independently selected from the group consisting of S, N, and O. In certain embodiments, A is optionally substituted, 6-membered heteroaryl with four heteroatoms each independently selected from the group consisting of S, N, and O.

As generally used herein, in Formulae (III) and (V), e is 0, 1, 2, or 3. In certain embodiments, e is 0. In certain embodiments, e is 1. In certain embodiments, e is 2. In certain embodiments, e is 3.

As generally used herein, each instance of R₄ is independently selected from the group consisting of hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(═O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, and —SO₂N(R^{B})₂. In some embodiments, each instance of R₄ is independently selected from the group consisting of hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A} and —C(═O)R^{A}. In some embodiments, each instance of R₄ is independently selected from the group consisting of hydrogen, halogen, —CN, —NO₂, —N₃, alkyl, alkenyl, alkynyl, carbocyclyl, aryl, heterocyclyl, heteroaryl, —OR^{A}, —N(R^{B})₂, —SR^{A} and —C(═O)R^{A}. In some embodiments, R₄ is halogen. In certain embodiments, R₄ is F. In certain embodiments, R₄ is Cl. In certain embodiments, R₄ is Br. In certain embodiments, R₄ is I. In some embodiments, R₄ is optionally substituted alkyl, optionally substituted alkenyl, optionally substituted alkynyl, or optionally substituted carbocyclyl. In certain embodiments, R₄ is optionally substituted C₁₋₆ alkyl, optionally substituted C₂₋₆ alkenyl, optionally substituted C₂₋₆ alkynyl, or optionally substituted C₃₋₆ carbocyclyl. In certain embodiments, R₄ is optionally substituted C₁₋₆ alkyl. In certain embodiments, R₄ is unsubstituted C₁₋₆ alkyl. In certain embodiments, R₄ is methyl. In certain embodiments, R₄ is ethyl. In certain embodiments, R₄ is propyl. In certain embodiments, R₄ is butyl. In certain embodiments, R₄ is pentyl. In certain embodiments, R₄ is isopropyl, isobutyl, or isoamyl. In certain embodiments, R₄ is tert-butyl. In some embodiments, R₄ is —CN. In some embodiments, R₄ is —NO₂. In some embodiments, R₄ is —N₃. In some embodiments, R₄ is optionally substituted carbocyclyl, optionally substituted aryl, optionally substituted heterocyclyl, or optionally substituted heteroaryl. In some embodiments, R₄ is cyclopropyl or cyclobutyl. In some embodiments, R₄ is —OR^{A}, —N(R^{B})₂, —SR^{A}, —C(═O)R^{A}, —C(O)OR^{A}, —C(O)SR^{A}, —C(O)N(R^{B})², —C(O)N(R^{B})N(R^{B})₂, —OC(O)R^{A}, —OC(O)N(R^{B})₂, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, or —SO₂N(R^{B})₂. In certain embodiments, R₄ is —N(R^{B})₂. In certain embodiments, R₄ is —NHR^{B}. In certain embodiments, R₄ is —NHR^{B}, wherein R^{B} is optionally substituted C₁₋₆ alkyl. In certain embodiments, R₄ is —NHR^{B}, wherein R^{B} is unsubstituted C₁₋₆ alkyl. In certain embodiment, R₄ is —NHR^{B}, wherein R^{B} is substituted C₁₋₆ alkyl. In certain embodiments, R₄ is —NH-methyl, —NH-ethyl, or —NH— propyl. In certain embodiments, R₄ is —N(R^{B})₂, wherein each R^{B} is independently optionally substituted C₁₋₆ alkyl. In certain embodiments, R₄ is —N(R^{B})₂, wherein each R^{B} is independently unsubstituted C₁₋₆ alkyl. In certain embodiments, R₄ is —N(R^{B})₂, wherein each R^{B} is independently selected from the group consisting of methyl, ethyl, isopropyl, isobutyl, isoamyl, and benzyl. In some embodiments, R₄ is —N(R^{B})₂, wherein each R^{B} is the same. In some embodiments, R₄ is —N(R^{B})₂, wherein each R^{B} is different. In certain embodiments, R₄ is —NH₂. In certain embodiments, R₂ is —OR^{A}. In certain embodiments, R₄ is —OH. In certain embodiments, R₄ is —OR^{A}, wherein R^{A} is optionally substituted alkyl, optionally substituted alkenyl, optionally substituted alkynyl, or optionally substituted carbocyclyl. In certain embodiments, R₄ is —OR^{A}, wherein R^{A} is optionally substituted C₁₋₆ alkyl. In certain embodiments, R₄ is —OR^{A}, wherein R^{A} is unsubstituted C₁₋₆ alkyl. In certain embodiments, R₄ is —O-methyl, —O-ethyl, or —O-propyl. In certain embodiments, R₄ is —OR^{A}, wherein R^{A} is optionally substituted aryl. In certain embodiments, R₄ is —O-phenyl. In certain embodiments, R₄ is substituted with —OR^{A}, wherein R^{A} is optionally substituted heteroaryl. In certain embodiments, R₄ is —C(═O)R^{A}, wherein R^{A} is optionally substituted alkyl, optionally substituted alkenyl, optionally substituted alkynyl, optionally substituted carbocyclyl, optionally substituted heterocyclyl, optionally substituted aryl, or optionally substituted heteroaryl. In certain embodiments, R₄ is —C(═O)Me.

As generally defined herein, R^{N4} is hydrogen, optionally substituted C₁₋₆ alkyl, or a nitrogen protecting group. In certain embodiments, R^{N4} is hydrogen. In certain embodiments, R^{N4} is optionally substituted C₁₋₆ alkyl. In certain embodiments, R^{N4} is a nitrogen protecting group. In certain embodiments, R^{N4} is acetyl, Bn, BOC, Cbz, Fmoc, or Ts.

As generally described above, B is independently hydrogen, optionally substituted alkyl, optionally substituted alkenyl, optionally substituted alkynyl, optionally substituted aryl, optionally substituted carbocyclyl, optionally substituted heterocyclyl, or optionally substituted heteroaryl. In certain embodiments, B is hydrogen. In certain embodiments, B is optionally substituted alkyl, optionally substituted aryl, optionally substituted carbocyclyl, optionally substituted heterocyclyl, optionally substituted 5-membered heteroaryl, or optionally substituted 6-membered heteroaryl. In certain embodiments, B is optionally substituted alkyl. In certain embodiments, B is substituted alkyl. In certain embodiments, B is unsubstituted alkyl. In certain embodiments, B is optionally substituted C₁₋₆ alkyl. In certain embodiments, B is optionally substituted methyl. In certain embodiments, B is optionally substituted ethyl. In certain embodiments, B is of the formula:

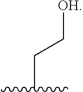 [SMILES: *CCO]

In certain embodiments, B is optionally substituted propyl. In certain embodiments, B is optionally substituted branched C₁₋₆ alkyl. In certain embodiments, B is substituted branched C₁₋₆ alkyl. In certain embodiments, B is unsubstituted branched C₁₋₆ alkyl. In certain embodiments, B is optionally substituted straight C₁₋₆ alkyl. In certain embodiments, B is substituted straight C₁₋₆ alkyl. In certain embodiments, B is unsubstituted straight C₁₋₆ alkyl. In certain embodiments, B is unsubstituted butyl. In certain embodiments, B is unsubstituted C₁₋₆ alkyl. In certain embodiments, B is methyl, ethyl, n-propyl, i-propyl, n-butyl, s-butyl, t-butyl, n-pentyl, t-pentyl, neo-pentyl, i-pentyl, s-pentyl, or 3-pentyl. In certain embodiments, B is methyl. In certain embodiments, B is ethyl. In certain embodiments, B is propyl. In certain embodiments, B is butyl. In certain embodiments, B is substituted C₁₋₆ alkyl. In certain embodiments, B is optionally substituted aryl. In certain embodiments, B is optionally substituted monocyclic aryl. In certain embodiments, B is optionally substituted phenyl. In certain embodiments, B is substituted phenyl. In certain embodiments, B is unsubstituted phenyl. In certain embodiments, B is substituted phenyl. In certain embodiments, B is mono-substituted phenyl. In certain embodiments, B is di-substituted phenyl. In certain embodiments, B is tri-substituted phenyl. In certain embodiments, B is tetra-substituted or penta-substituted phenyl.

In certain embodiments, B is of formula:

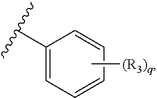

wherein each instance of R₃ is independently selected from the group consisting of hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(═O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, and —SO₂N(R^{B})₂. In certain embodiments, B is optionally substituted benzyl. In certain embodiments, B is of formula:

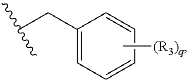

wherein each instance of R₃ is independently selected from the group consisting of hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(═O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, and —SO₂N(R^{B})₂. In certain embodiments, each instance of R₃ is independently selected from the group consisting of hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, and —C(═O)R^{A}. In certain embodiments, each instance of R₃ is independently selected from the group consisting of hydrogen, halogen, —CN, —NO₂, —N₃, alkyl, alkenyl, alkynyl, carbocyclyl, aryl, heterocyclyl, heteroaryl, —OR^{A}, —N(R^{B})₂, —SR^{A}, and —C(═O)R^{A}.

In certain embodiments, R₃ is hydrogen. In some embodiments, R₃ is halogen. In certain embodiments, R₃ is F. In certain embodiments, R₃ is Cl. In certain embodiments, R₃ is Br. In certain embodiments, R₃ is I. In some embodiments, R₃ is optionally substituted alkyl, optionally substituted alkenyl, optionally substituted alkynyl, or optionally substituted carbocyclyl. In certain embodiments, R₃ is optionally substituted C₁₋₆ alkyl, optionally substituted C₂₋₆ alkenyl, optionally substituted C₂₋₆ alkynyl, or optionally substituted C₃₋₆ carbocyclyl. In certain embodiments, R₃ is optionally substituted C₁₋₆ alkyl. In certain embodiments, R₃ is methyl. In certain embodiments, R₃ is ethyl. In certain embodiments, R₃ is propyl. In certain embodiments, R₃ is butyl. In certain embodiments, R₃ is pentyl. In certain embodiments, R₃ is isopropyl, isobutyl, or isoamyl. In certain embodiments, R₃ is tert-butyl. In some embodiments, R₃ is —CN. In some embodiments, R₃ is —NO₂. In some embodiments, R₃ is —N₃. In some embodiments, R₃ is optionally substituted carbocyclyl, optionally substituted aryl, optionally substituted heterocyclyl, or optionally substituted heteroaryl. In some embodiments, R₃ is cyclopropyl or cyclobutyl. In some embodiments, R₃ is —OR^{A}, —N(R^{B})₂, —SR^{A}, —C(═O)R^{A}, —C(O)OR^{A}, —C(O)SR^{A}, —C(O)N(R^{B})₂, —C(O)N(R^{B})N(R^{B})₂, —OC(O)R^{A}, —OC(O)N(R^{B})₂, —NR^{B}C(O)N(R^{B})₂, —NR^{B}C(O)N(R^{B})N(R^{B})₂, —NR^{B}C(O)OR^{A}, —SC(O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(O)R^{A}, —OS(O)₂R^{A}, —SO₂R^{A}, or —SO₂N(R^{B})₂. In certain embodiments, R₃ is —N(R^{B})₂. In certain embodiments, R₃ is —NHR^{B}. In certain embodiments, R₃ is —NHR^{B}, wherein R^{B} is optionally substituted C₁₋₆ alkyl. In certain embodiments, R₃ is —NHR^{B}, wherein R^{B} is unsubstituted C₁₋₆ alkyl. In certain embodiment, R₃ is —NHR^{B}, wherein R^{B} is methyl, ethyl, or propyl. In certain embodiments, R₃ is —N(R^{B})₂, wherein each R^{B} is independently optionally substituted C₁₋₆ alkyl. In certain embodiments, R₃ is —N(R^{B})₂, wherein each R^{B} is independently selected from the group consisting of methyl, ethyl, isopropyl, isobutyl, isoamyl, and benzyl. In some embodiments, R₃ is —N(R^{B})₂, wherein each R^{B} is the same. In some embodiments, R₃ is —N(R^{B})₂, wherein each R^{B} is different. In certain embodiments, R₃ is —NH₂. In certain embodiments, R₃ is —OR^{A}. In certain embodiments, R₃ is —OH. In certain embodiments, R₃ is —OR^{A}, wherein R^{A} is optionally substituted alkyl, optionally substituted alkenyl, optionally substituted alkynyl, or optionally substituted carbocyclyl. In certain embodiments, R₃ is —OR^{A}, wherein R^{A} is optionally substituted C₁₋₆ alkyl. In certain embodiments, R₃ is —OR^{A}, wherein R^{A} is unsubstituted C₁₋₆ alkyl. In certain embodiments, R₃ is —O-methyl, —O-ethyl, or —O-propyl. In certain embodiments, R₃ is —O-methyl. In certain embodiments, R₃ is —OR^{A}, wherein R^{A} is optionally substituted aryl. In certain embodiments, R₃ is —O-phenyl. In certain embodiments, R₃ is substituted with —OR^{A}, wherein R^{A} is optionally substituted heteroaryl.

In certain embodiments, q is 0. In certain embodiments, B is

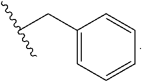 [SMILES: *Cc1ccccc1]

In certain embodiments, q is 1. In certain embodiments, B is

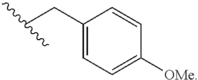 [SMILES: *Cc1ccc(OC)cc1]

In certain embodiments, B is

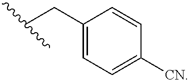 [SMILES: *Cc1ccc(C#N)cc1]

In certain embodiments, q is 2. In certain embodiments, q is 3. In certain embodiments, q is 4. In certain embodiments, q is 5.

As generally defined herein, q is 0, 1, 2, 3, 4, or 5. In certain embodiments, q is 0 and B is unsubstituted phenyl. In certain embodiments, R₃ is hydrogen, q is 5, and B is unsubstituted phenyl. In certain embodiments, q is 1. In certain embodiments, B is of the formula:

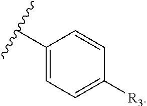

In certain embodiments, B is of the formula:

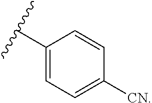 [SMILES: *c1ccc(C#N)cc1]

In certain embodiments, B is of the formula:

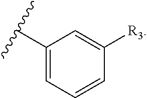

In certain embodiments, B is of the formula:

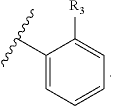

In certain embodiments, q is 2. In certain embodiments, B is of the formula:

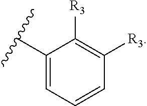

In certain embodiments, B is of the formula:

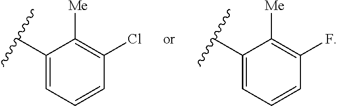 [SMILES: *c1cccc(Cl)c1C ; *c1cccc(F)c1C]

In certain embodiments, B is of the formula:

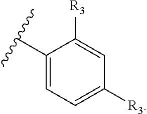

In certain embodiments, B is of the formula:

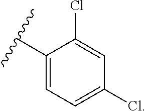 [SMILES: *c1ccc(Cl)cc1Cl]

In certain embodiments, B is of the formula:

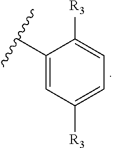

In certain embodiments, B is of the formula:

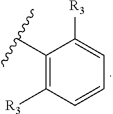

In certain embodiments, B is of the formula:

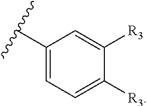

In certain embodiments, B is of the formula:

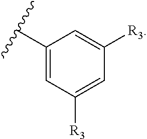

In certain embodiments, q is 3. In certain embodiments, B is of the formula:

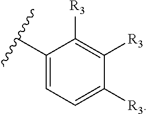

In certain embodiments, B is of the formula:

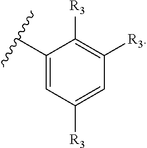

In certain embodiments, B is of the formula:

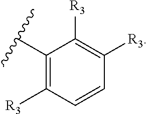

In certain embodiments, B is of the formula:

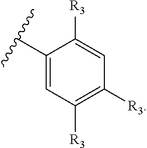

In certain embodiments, B is of the formula:

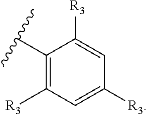

In certain embodiments, q is 4. In certain embodiments, B is of the formula:

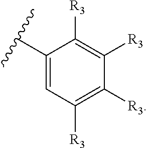

In certain embodiments, B is of the formula:

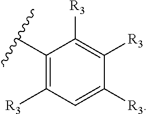

In certain embodiments, B is of the formula:

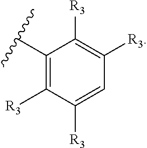

In certain embodiments, q is 5. In certain embodiments, B is of the formula:

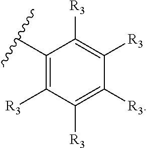

In certain embodiments, q is 1, and R₃ is optionally substituted C₁₋₆ alkyl, optionally substituted C₂₋₆ alkenyl, or —OR^{A}. In certain embodiments, q is 1, and R₃ is optionally substituted C₁₋₆ alkyl, optionally substituted C₂₋₆ alkenyl, or —OR^{A} at the ortho-position of the phenyl ring. In certain embodiments, q is 1, and R₃ is optionally substituted C₁₋₆ alkyl, optionally substituted C₂₋₆ alkenyl, or —OR^{A} at the meta-position of the phenyl ring. In certain embodiments, q is 1, and R₃ is optionally substituted C₁₋₆ alkyl, optionally substituted C₂₋₆ alkenyl, or —OR^{A} at the para-position of the phenyl ring. In certain embodiments, q is 1, and R₃ is methyl, ethyl, isopropyl, tert-butyl, allyl, F, Cl, Br, I, —OH, methoxy, or ethoxy. In certain embodiments, q is 2, and each instance of R₃ is optionally substituted C₁₋₆ alkyl, optionally substituted C₂₋₆ alkenyl, or —OR^{A}. In certain embodiments, q is 2, and each instance of R₃ is independently optionally substituted C₁₋₆ alkyl, optionally substituted C₂₋₆ alkenyl, or —OR^{A}, wherein R^{A} is hydrogen, optionally substituted C₁₋₆ alkyl, or an oxygen protecting group. In certain embodiments, q is 2, and each instance of R₃ is independently methyl, ethyl, propyl, isopropyl, tert-butyl, allyl, —OH, methoxy, or ethoxy. In certain embodiments, q is 3, and each instance of R₃ is optionally substituted C₁₋₆ alkyl, optionally substituted C₂₋₆ alkenyl, or —OR^{A}. In certain embodiments, q is 3, and each instance of R₃ is independently optionally substituted C₁₋₆ alkyl, optionally substituted C₂₋₆ alkenyl, or —OR^{A}, wherein R^{A} is hydrogen, optionally substituted C₁₋₆ alkyl, or an oxygen protecting group. In certain embodiments, q is 3, and each instance of R₃ is independently methyl, ethyl, propyl, isopropyl, tert-butyl, allyl, —OH, methoxy, or ethoxy. In certain embodiments, q is 4, and each instance of R₃ is optionally substituted C₁₋₆ alkyl, optionally substituted C₂₋₆ alkenyl, or —OR^{A}. In certain embodiments, q is 4, and each instance of R₃ is independently optionally substituted C₁₋₆ alkyl, optionally substituted C₂₋₆ alkenyl, or —OR^{A}, wherein R^{A} is hydrogen, optionally substituted C₁₋₆ alkyl, or an oxygen protecting group. In certain embodiments, q is 4 and each instance of R₃ is independently methyl, ethyl, propyl, iso-propyl, tert-butyl, allyl, —OH, methoxy, or ethoxy. In certain embodiments, q is 5, and each instance of R₃ is optionally substituted C₁₋₆ alkyl, optionally substituted C₂₋₆ alkenyl, or —OR^{A}. In certain embodiments, q is 5, and each instance of R₃ is independently optionally substituted C₁₋₆ alkyl, optionally substituted C₂₋₆ alkenyl, or —OR^{A}, wherein R^{A} is hydrogen, optionally substituted C₁₋₆ alkyl, or an oxygen protecting group. In certain embodiments, q is 5, and each instance of R₃ is independently methyl, ethyl, propyl, iso-propyl, tert-butyl, allyl, —OH, methoxy, or ethoxy.

In certain embodiments, B is optionally substituted heteroaryl. In certain embodiments, B is optionally substituted 5-membered heteroaryl, or optionally substituted 6-membered heteroaryl. In certain embodiments, B is unsubstituted 5-membered heteroaryl. In certain embodiments, B is substituted 5-membered heteroaryl. In certain embodiments, B is optionally substituted, 5-membered heteroaryl with one heteroatom selected from the group consisting of S, N, and O. In certain embodiments, B is optionally substituted 5-membered heteroaryl with two heteroatoms each independently selected from the group consisting of S, N, and O. In certain embodiments, B is optionally substituted, 5-membered heteroaryl with three heteroatoms each independently selected from the group consisting of S, N, and O. In certain embodiments, B is optionally substituted 5-membered heteroaryl with four heteroatoms each independently selected from the group consisting of S, N, and O.

In certain embodiments, B is optionally substituted, 6-membered heteroaryl. In certain embodiments, B is unsubstituted, 6-membered heteroaryl. In certain embodiments, B is substituted, 6-membered heteroaryl. In certain embodiments, B is optionally substituted, 5-membered heteroaryl with one heteroatom selected from the group consisting of S, N, and O. In certain embodiments, B is optionally substituted, 6-membered heteroaryl with one heteroatom selected from the group consisting of S, N, and O. In certain embodiments, B is optionally substituted, 6-membered heteroaryl with two heteroatoms each independently selected from the group consisting of S, N, and O. In certain embodiments, B is optionally substituted, 6-membered heteroaryl with three heteroatoms each independently selected from the group consisting of S, N, and O. In certain embodiments, B is optionally substituted, 6-membered heteroaryl with four heteroatoms each independently selected from the group consisting of S, N, and O.

In certain embodiments, B is of Formula (b-1):

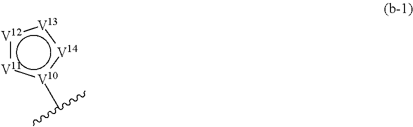

wherein each instance of V¹⁰, V¹¹, V¹², V¹³, and V¹⁴ is independently O, S, C, N, NR^{A1}, or CR^{A2}, as valency permits, wherein R^{A1} and R^{A2} are as defined herein.

In certain embodiments of Formula (b-1), V¹⁰, V¹¹, V¹², V¹³, and V¹⁴ may each independently be O, S, N, NR^{A1}, C, or CR^{A2}, as valency permits. In certain embodiments, only one of V¹⁰, V¹¹, V¹², V¹³, and V¹⁴ is selected from the group consisting of O, S, N, and NR^{A1}. In certain embodiments, B is of the formula:

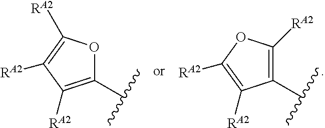

In certain embodiments, B is of the formula:

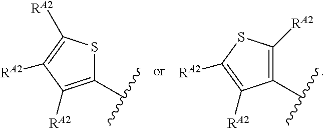

In certain embodiments, B is of the formula:

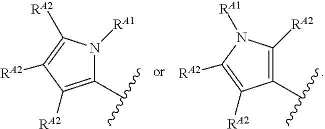

In certain embodiments, only two of V¹⁰, V¹¹, V¹², V¹³, and V¹⁴ are each independently selected from the group consisting of O, S, N, and NR^{A1}. In certain embodiments, B is of the formula:

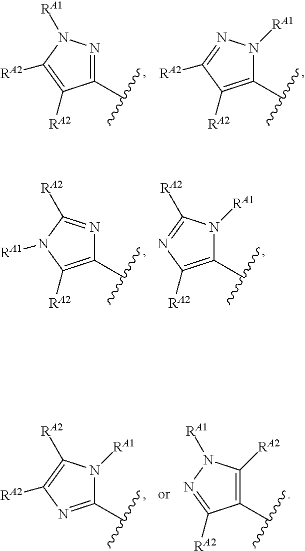

In certain embodiments, B is of the formula:

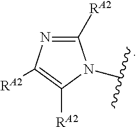

In certain embodiments, B is of the formula:

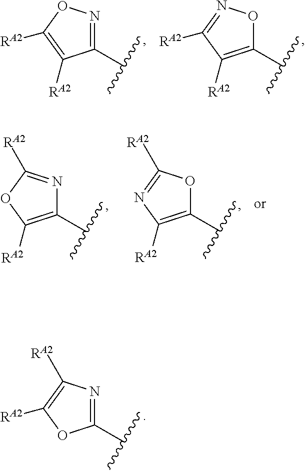

In certain embodiments, B is of the formula:

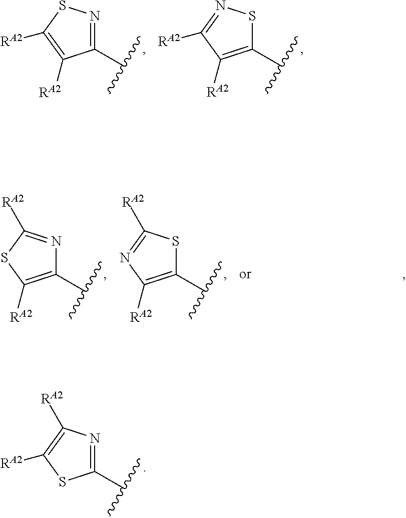

In certain embodiments, only three of V¹⁰, V¹¹, V¹², V¹³, and V¹⁴ are each independently selected from the group consisting of O, S, N, and NR^{A1}. In certain embodiments, B is of the formula:

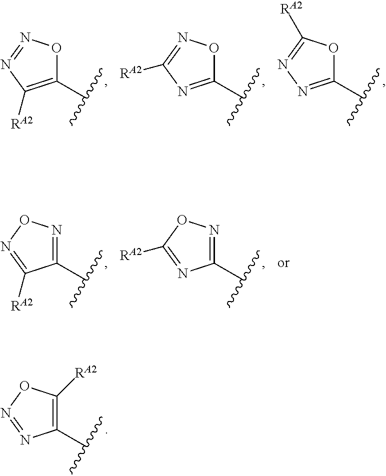

In certain embodiments, B is of the formula:

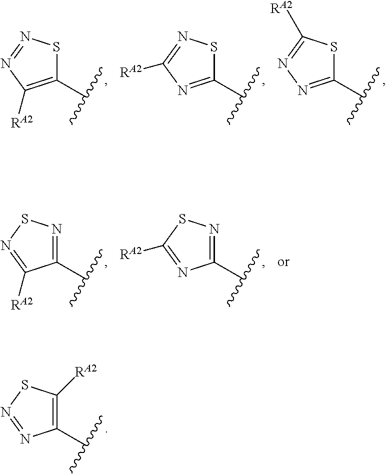

In certain embodiments, B is of the formula:

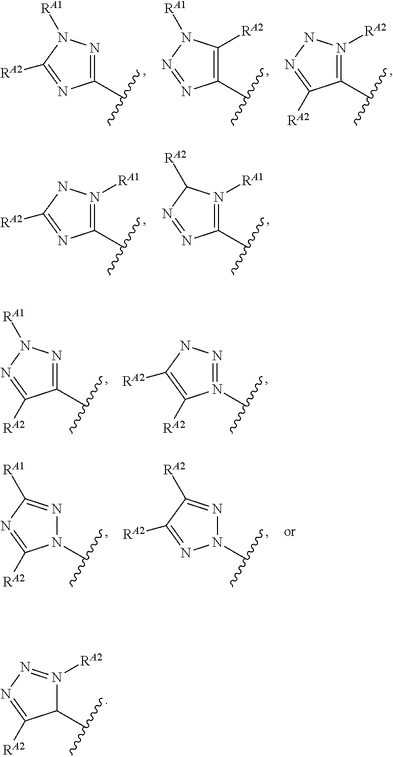

In certain embodiments, only four of V¹⁰, V¹¹, V¹², V¹³, and V¹⁴ are each independently selected from the group consisting of N and NR^{A1}. In certain embodiments, B is of the formula:

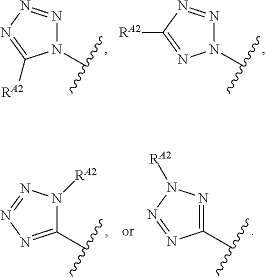

In certain embodiments, B is of Formula (b-2):

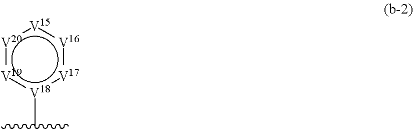

wherein each instance of V¹⁵, V¹⁶, V¹⁷, V¹⁸, V¹⁹, and V²⁰ is independently N, C, or CR^{A2}, as valency permits, wherein R^{A1} and R^{A2} are as defined herein.

In certain embodiments of Formula (b-2), V¹⁵-V²⁰ may each independently be N, C, or CR^{A2}, as valency permits. In certain embodiments, only one of V¹⁵-V²⁰ is N. In certain embodiments, B is of one of the following formulae:

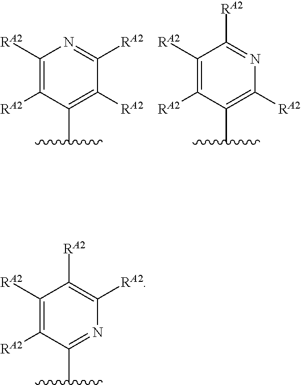

In certain embodiments, only two of V¹⁵-V²⁰ are N. In certain embodiments, B is of one of the following formulae:

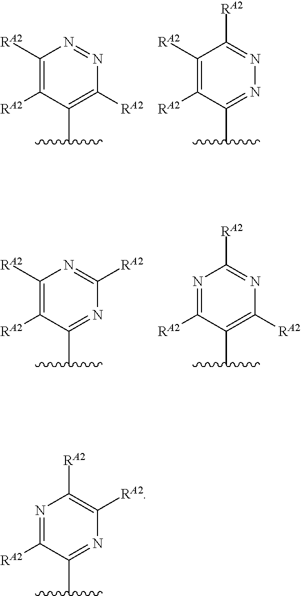

In certain embodiments, B is

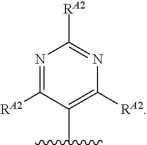

In certain embodiments, B is

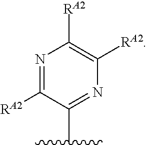

As generally defined herein, each instance of R^{A1} is independently selected from the group consisting of hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group.

As generally defined herein, each instance of R^{A2} is independently selected from the group consisting of hydrogen, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —OR^{A2a}, —N(R^{A2a})₂, and —SR^{A2a}, wherein each occurrence of R^{A2a} is independently selected from the group consisting of hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, and a sulfur protecting group when attached to a sulfur atom, or two R^{A2a} groups are joined to form an optionally substituted heterocyclic ring.

In certain embodiments, optionally any two of R^{A1}, R^{A2}, and R^{A2a} groups are joined to form an optionally substituted carbocyclic, optionally substituted heterocyclic, optionally substituted aryl, or optionally substituted heteroaryl ring. In certain embodiments, R^{A1} and R^{A2} are joined to form an optionally substituted carbocyclic, optionally substituted heterocyclic, optionally substituted aryl, or optionally substituted heteroaryl ring. In certain embodiments, R^{A1} and R^{A2a} are joined to form an optionally substituted carbocyclic, optionally substituted heterocyclic, optionally substituted aryl, or optionally substituted heteroaryl ring. In certain embodiments, R^{A2} and R^{A2a} are joined to form an optionally substituted carbocyclic, optionally substituted heterocyclic, optionally substituted aryl, or optionally substituted heteroaryl ring.

In certain embodiments, at least one R^{A1} is hydrogen, C₁₋₆ alkyl, or a nitrogen protecting group. In certain embodiments, all instances of R^{A1} are each independently hydrogen, C₁₋₆ alkyl, or a nitrogen protecting group. In certain embodiments, all instances of R^{A1} are hydrogen.

In certain embodiments, at least one R^{A2} is hydrogen, optionally substituted C₁₋₆ alkyl, halogen, or a nitrogen protecting group. In certain embodiments, all instances of R^{A2} are each independently hydrogen, halogen, optionally substituted C₁₋₆ alkyl, or a nitrogen protecting group. In certain embodiments, all instances of R^{A2} are hydrogen.

In certain embodiments, only one instance of R^{A2} is optionally substituted C₁₋₆ alkyl. In certain embodiments, only one instance of R^{A2} is unsubstituted C₁₋₆ alkyl (e.g., methyl). In certain embodiments, only one instance of R^{A2} is halogen. In certain embodiments, only one instance of R^{A2} is F. In certain embodiments, only one instance of R^{A2} is Cl. In certain embodiments, only one instance of R^{A2} is Br. In certain embodiments, only one instance of R^{A2} is I.

In certain embodiments, only two instances of R^{A2} are optionally substituted C₁₋₆ alkyl. In certain embodiments, only two instances of R^{A2} are unsubstituted C₁₋₆ alkyl (e.g., methyl). In certain embodiments, only two instances of R^{A2} are halogen.

In certain embodiments, B is optionally substituted carbocyclyl. In certain embodiments, B is optionally substituted monocyclic carbocyclyl. In some embodiments, B is optionally substituted C₃₋₆ carbocyclyl. In some embodiments, B is an optionally substituted 6-membered monocyclic carbocyclyl. In some embodiments, B is an optionally substituted bicyclic carbocyclyl. In some embodiments, B is optionally substituted cyclopentyl. In some embodiments, B is optionally substituted cyclohexyl.

In some embodiments, B is optionally substituted heterocyclyl. In some embodiments, B is optionally substituted, monocyclic heterocyclyl. In some embodiments, B is optionally substituted, six-membered heterocyclyl. In some embodiments, B is optionally substituted, bicyclic heterocyclyl. In some embodiments, B is optionally substituted, 3- to 6-membered monocyclic heterocyclyl. In some embodiments, B is optionally substituted, 6-membered heterocyclyl with one heteroatom selected from the group consisting of N, O, and S. In some embodiments, B is optionally substituted, 5-membered heterocyclyl with one heteroatom selected from the group consisting of N, O, and S.

In some embodiments, B is of one of the following formulae:

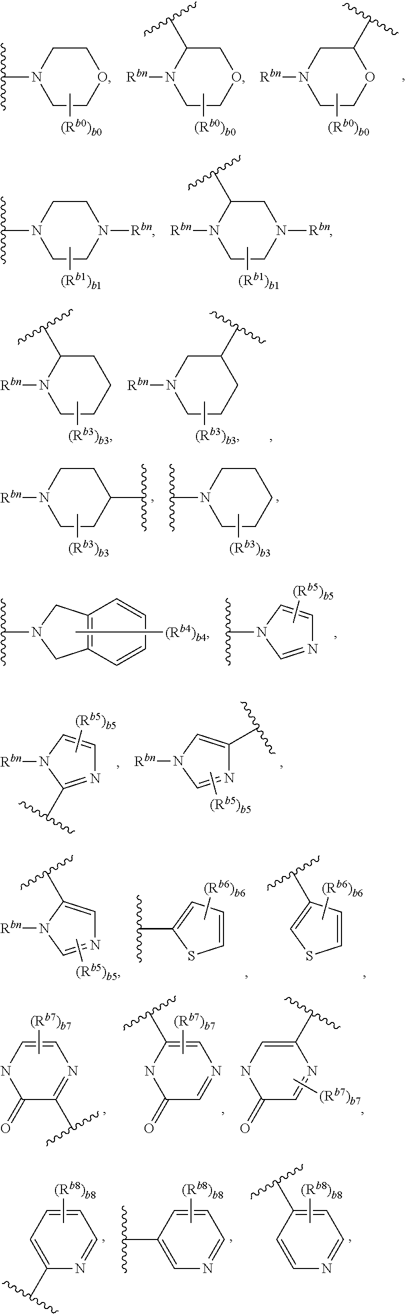

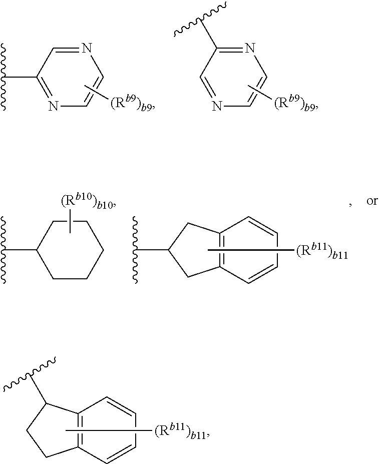

wherein R^{b0}, R^{b1}, R^{b3}, R^{b4}, R^{b5}, R^{b6}, R^{b7}, R^{b8}, R^{b9}, R^{b10}, R^{b11}, R^{bn}, b0, b1, b3, b4, b5, b6, b7, b8, b9, b10, and b11 are as defined herein. In some embodiments, B is

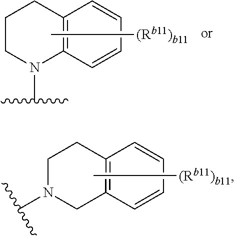

wherein R^{b11}, and b11 are as defined herein.

As generally defined herein, each instance of R^{b0}, R^{b1}, R^{b2}, R^{b3}, R^{b4}, R^{b5}, R^{b6}, R^{b7}, R^{b8}, R^{b9}, R^{b10}, and R^{b11} is independently selected from the group consisting of hydrogen, halogen, —CN, —NO₂, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, or —SO₂N(R^{B})₂, wherein R^{A} and R^{B} are as defined herein. In certain embodiments, each instance of R^{b0}, R^{b1}, R^{b2}, R^{b3}, R^{b4}, R^{b5}, R^{b6}, R^{b7}, R^{b8}, R^{b9}, R^{b10}, and R^{b11} is independently selected from the group consisting of hydrogen, halogen, —CN, —NO₂, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, and —C(═O)R^{A}. In certain embodiments, each instance of R^{b0}, R^{b1}, R^{b2}, R^{b3}, R^{b4}, R^{b5}, R^{b6}, R^{b7}, R^{b8}, R^{b9}, R^{b10}, and R^{b11} is independently selected from the group consisting of hydrogen, halogen, —CN, —NO₂, alkyl, alkenyl, alkynyl, carbocyclyl, optionally substituted aryl, heterocyclyl, heteroaryl, —OR^{A}, —N(R^{B})₂, —SR^{A}, and —C(═O)R^{A}.

In certain embodiments, R^{b0} is hydrogen, halogen, optionally substituted C₁₋₆ alkyl, —OR^{A}, or —C(═O)R^{A}. In certain embodiments, R^{b0} is hydrogen. In certain embodiments, R^{b0} is halogen (e.g., F, Cl). In certain embodiments, R^{b0} is optionally substituted C₁₋₆ alkyl. In certain embodiments, R^{b0} is unsubstituted C₁₋₆ alkyl (e.g., methyl, ethyl). In certain embodiments, R^{b0} is substituted C₁₋₆ alkyl (e.g., Bn). In certain embodiments, R^{b0} is —OR^{A}, wherein R^{A} is as generally defined herein. In certain embodiments, R^{b0} is —OR^{A}, wherein R^{A} is hydrogen, optionally substituted C₁₋₆ alkyl, or an oxygen protecting group.

In certain embodiments, R^{b1} is hydrogen, halogen, optionally substituted C₁₋₆ alkyl, —OR^{A}, or —C(═O)R^{A}. In certain embodiments, R^{b1} is hydrogen. In certain embodiments, R^{b1} is halogen (e.g., F, Cl). In certain embodiments, R^{b1} is optionally substituted C₁₋₆ alkyl. In certain embodiments, R^{b1} is unsubstituted C₁₋₆ alkyl (e.g., methyl, ethyl). In certain embodiments, R^{b1} is substituted C₁₋₆ alkyl (e.g., Bn). In certain embodiments, R^{b1} is —OR^{A}, wherein R^{A} is as generally defined herein. In certain embodiments, R^{b1} is —OR^{A}, wherein R^{A} is hydrogen, optionally substituted C₁₋₆ alkyl, or an oxygen protecting group.

In certain embodiments, R^{b3} is hydrogen, halogen, optionally substituted C₁₋₆ alkyl, —OR^{A}, or —C(═O)R^{A}. In certain embodiments, R^{b3} is hydrogen. In certain embodiments, R^{b3} is halogen (e.g., F, Cl). In certain embodiments, R^{b3} is optionally substituted C₁₋₆ alkyl. In certain embodiments, R^{b3} is unsubstituted C₁₋₆ alkyl (e.g., methyl, ethyl). In certain embodiments, R^{b3} is substituted C₁₋₆ alkyl (e.g., Bn). In certain embodiments, R^{b3} is —OR^{A}, wherein R^{A} is as generally defined herein. In certain embodiments, R^{b3} is —OR^{A}, wherein R^{A} is hydrogen, optionally substituted C₁₋₆ alkyl, or an oxygen protecting group.

In certain embodiments, R^{b4} is hydrogen, halogen, optionally substituted C₁₋₆ alkyl, —OR^{A}, or —C(═O)R^{A}. In certain embodiments, R^{b4} is hydrogen. In certain embodiments, R^{b4} is halogen (e.g., F, Cl). In certain embodiments, R^{b4} is optionally substituted C₁₋₆ alkyl. In certain embodiments, R^{b4} is unsubstituted C₁₋₆ alkyl (e.g., methyl, ethyl). In certain embodiments, R^{b4} is substituted C₁₋₆ alkyl (e.g., Bn). In certain embodiments, R^{b4} is —OR^{A}, wherein R^{A} is as generally defined herein. In certain embodiments, R^{b4} is —OR^{A}, wherein R^{A} is hydrogen, optionally substituted C₁₋₆ alkyl, or an oxygen protecting group.

In certain embodiments, R^{b5} is hydrogen, halogen, optionally substituted C₁₋₆ alkyl, —OR^{A}, or —C(═O)R^{A}. In certain embodiments, R^{b5} is hydrogen. In certain embodiments, R^{b5} is halogen (e.g., F, Cl). In certain embodiments, R^{b5} is optionally substituted C₁₋₆ alkyl. In certain embodiments, R^{b5} is unsubstituted C₁₋₆ alkyl (e.g., methyl, ethyl). In certain embodiments, R^{b5} is substituted C₁₋₆ alkyl (e.g., Bn). In certain embodiments, R^{b5} is —OR^{A}, wherein R^{A} is as generally defined herein. In certain embodiments, R^{b5} is —OR^{A}, wherein R^{A} is hydrogen, optionally substituted C₁₋₆ alkyl, or an oxygen protecting group.

In certain embodiments, R^{b6} is hydrogen, halogen, optionally substituted C₁₋₆ alkyl, —OR^{A}, or —C(═O)R^{A}. In certain embodiments, R^{b6} is hydrogen. In certain embodiments, R^{b6} is halogen (e.g., F, Cl). In certain embodiments, R^{b6} is optionally substituted C₁₋₆ alkyl. In certain embodiments, R^{b6} is unsubstituted C₁₋₆ alkyl (e.g., methyl, ethyl). In certain embodiments, R^{b6} is substituted C₁₋₆ alkyl (e.g., Bn). In certain embodiments, R^{b6} is —OR^{A}, wherein R^{A} is as generally defined herein. In certain embodiments, R^{b6} is —OR^{A}, wherein R^{A} is hydrogen, optionally substituted C₁₋₆ alkyl, or an oxygen protecting group.

In certain embodiments, R^{b7} is hydrogen, halogen, optionally substituted C₁₋₆ alkyl, —OR^{A}, or —C(═O)R^{A}. In certain embodiments, R^{b7} is hydrogen. In certain embodiments, R^{b7} is halogen (e.g., F, Cl). In certain embodiments, R^{b7} is optionally substituted C₁₋₆ alkyl. In certain embodiments, R^{b7} is unsubstituted C₁₋₆ alkyl (e.g., methyl, ethyl). In certain embodiments, R^{b7} is substituted C₁₋₆ alkyl (e.g., Bn). In certain embodiments, R^{b7} is —OR^{A}, wherein R^{A} is as generally defined herein. In certain embodiments, R^{b7} is —OR^{A}, wherein R^{A} is hydrogen, optionally substituted C₁₋₆ alkyl, or an oxygen protecting group.

In certain embodiments, R^{b8} is hydrogen, halogen, optionally substituted C₁₋₆ alkyl, —OR^{A}, or —C(═O)R^{A}. In certain embodiments, R^{b8} is hydrogen. In certain embodiments, R^{b8} is halogen (e.g., F, Cl). In certain embodiments, R^{b8} is optionally substituted C₁₋₆ alkyl. In certain embodiments, R^{b8} is unsubstituted C₁₋₆ alkyl (e.g., methyl, ethyl). In certain embodiments, R^{b8} is substituted C₁₋₆ alkyl (e.g., Bn). In certain embodiments, R^{b8} is —OR^{A}, wherein R^{A} is as generally defined herein. In certain embodiments, Rb is —OR^{A}, wherein R^{A} is hydrogen, optionally substituted C₁₋₆ alkyl, or an oxygen protecting group.

In certain embodiments, R^{b9} is hydrogen, halogen, optionally substituted C₁₋₆ alkyl, —OR^{A}, or —C(═O)R^{A}. In certain embodiments, R^{b9} is hydrogen. In certain embodiments, R^{b9} is halogen (e.g., F, Cl). In certain embodiments, R^{b9} is optionally substituted C₁₋₆ alkyl. In certain embodiments, R^{b9} is unsubstituted C₁₋₆ alkyl (e.g., methyl, ethyl). In certain embodiments, R^{b9} is substituted C₁₋₆ alkyl (e.g., Bn). In certain embodiments, R^{b9} is —OR^{A}, wherein R^{A} is as generally defined herein. In certain embodiments, R^{b9} is —OR^{A}, wherein R^{A} is hydrogen, optionally substituted C₁₋₆ alkyl, or an oxygen protecting group.

In certain embodiments, R^{b10} is hydrogen, halogen, optionally substituted C₁₋₆ alkyl, —OR^{A}, or —C(═O)R^{A}. In certain embodiments, at least one instance of R^{b10} is hydrogen. In certain embodiments, all instances of R^{b10} is hydrogen. In certain embodiments, at least one instance of R^{b10} is halogen (e.g., F, Cl). In certain embodiments, at least one instance of R^{b10} is optionally substituted C₁₋₆ alkyl. In certain embodiments, at least one instance of R^{b10} is unsubstituted C₁₋₆ alkyl (e.g., methyl, ethyl). In certain embodiments, at least one instance of R^{b10} is substituted C₁₋₆ alkyl (e.g., Bn). In certain embodiments, at least one instance of R^{b10} is —OR^{A}, wherein R^{A} is as generally defined herein. In certain embodiments, at least one instance of R^{b10} is —OR^{A}, wherein R^{A} is hydrogen, optionally substituted C₁₋₆ alkyl, or an oxygen protecting group.

In certain embodiments, R^{b11} is hydrogen, halogen, optionally substituted C₁₋₆ alkyl, —OR^{A}, or —C(═O)R^{A}. In certain embodiments, at least one instance of R^{b1} is hydrogen. In certain embodiments, at least one instance of R^{b1} is halogen (e.g., F, Cl). In certain embodiments, at least one instance of R^{b11} is optionally substituted C₁₋₆ alkyl. In certain embodiments, at least one instance of R^{b11} is unsubstituted C₁₋₆ alkyl (e.g., methyl, ethyl). In certain embodiments, at least one instance of R^{b11} is substituted C₁₋₆ alkyl (e.g., Bn). In certain embodiments, at least one instance of R^{b11} is —OR^{A}, wherein R^{A} is as generally defined herein. In certain embodiments, at least one instance of R^{b11} is —OR^{A}, wherein R^{A} is hydrogen, optionally substituted C₁₋₆ alkyl, or an oxygen protecting group.

As generally defined herein, each instance of R^{bn} is independently hydrogen, optionally substituted alkyl, optionally substituted alkenyl, optionally substituted alkynyl, optionally substituted carbocyclyl, optionally substituted aryl, optionally substituted heterocyclyl, optionally substituted heteroaryl, —C(═O)R^{b2}, or a nitrogen protecting group, wherein each instance of R^{b2} is independently selected from the group consisting of hydrogen, optionally substituted alkyl, optionally substituted alkenyl, optionally substituted alkynyl, optionally substituted carbocyclyl, optionally substituted aryl, optionally substituted heterocyclyl, or optionally substituted heteroaryl. In certain embodiments, R^{bn} is hydrogen. In certain embodiments, R^{bn} is optionally substituted C₁₋₆ alkyl. In certain embodiments, R^{bn} is unsubstituted C₁₋₆ alkyl (e.g. methyl or ethyl). In certain embodiments, R^{bn} is substituted C₁₋₆ alkyl. In certain embodiments, R^{bn} is optionally substituted heteroaryl-C₁₋₆ alkyl. In certain embodiments, R^{bn} is optionally substituted aryl-C₁₋₆ alkyl. In certain embodiments, R^{bn} is optionally substituted phenyl-C₁₋₆ alkyl. In certain embodiments, R^{bn} is Bn. In certain embodiments, R^{bn} is —C(═O)R^{b2}, wherein R^{b2} is as generally defined herein. In certain embodiments, R^{bn} is —C(═O)R^{b2}, wherein R^{b2} is optionally substituted aryl or optionally substituted heteroaryl.

In certain embodiments, R^{b2} is hydrogen, halogen, optionally substituted C₁₋₆ alkyl, —OR^{A}, or —C(═O)R^{A}. In certain embodiments, R^{b2} is hydrogen. In certain embodiments, R^{b2} is halogen (e.g., F, Cl). In certain embodiments, R^{b2} is optionally substituted C₁₋₆ alkyl. In certain embodiments, R^{b2} is unsubstituted C₁₋₆ alkyl (e.g., methyl, ethyl). In certain embodiments, R^{b2} is substituted C₁₋₆ alkyl (e.g., Bn). In certain embodiments, R^{b2} is —OR^{A}, wherein R^{A} is as generally defined herein. In certain embodiments, R^{b2} is —OR^{A}, wherein R^{A} is hydrogen, optionally substituted C₁₋₆ alkyl, or an oxygen protecting group.

As generally defined herein, b0 is independently 0, 1, 2, 3, 4, 5, 6, 7, or 8. In certain embodiments, b0 is 0. In certain embodiments, b0 is 1. In certain embodiments, b0 is 2. In certain embodiments, b0 is 3. In certain embodiments, b0 is 4.

As generally defined herein, b is independently 0, 1, 2, 3, 4, 5, 6, 7, or 8. In certain embodiments, b1 is 0. In certain embodiments, b1 is 1. In certain embodiments, b1 is 2. In certain embodiments, b is 3. In certain embodiments, b is 4.

As generally defined herein, b3 is independently 0 or an integer of 1 to 10, inclusive. In certain embodiments, b3 is 0. In certain embodiments, b3 is 1. In certain embodiments, b3 is 2. In certain embodiments, b3 is 3. In certain embodiments, b3 is 4.

As generally defined herein, b4 is independently 0, 1, 2, 3, 4, 5, 6, 7, or 8. In certain embodiments, b4 is 0. In certain embodiments, b4 is 1. In certain embodiments, b4 is 2. In certain embodiments, b4 is 3. In certain embodiments, b4 is 4.

As generally defined herein, b5 is independently 0, 1, 2, or 3. In certain embodiments, b5 is 0. In certain embodiments, b5 is 1. In certain embodiments, b5 is 2. In certain embodiments, b5 is 3.

As generally defined herein, b6 is independently 0, 1, 2, or 3. In certain embodiments, b6 is 0. In certain embodiments, b6 is 1. In certain embodiments, b6 is 2. In certain embodiments, b6 is 3.

As generally defined herein, b7 is independently 0, 1, 2, or 3. In certain embodiments, b6 is 0. In certain embodiments, b7 is 1. In certain embodiments, b7 is 2. In certain embodiments, b7 is 3.

As generally defined herein, b8 is independently 0, 1, 2, 3, or 4. In certain embodiments, b6 is 0. In certain embodiments, b8 is 1. In certain embodiments, b8 is 2. In certain embodiments, b8 is 3. In certain embodiments, b8 is 4.

As generally defined herein, b9 is independently 0, 1, 2, or 3. In certain embodiments, b6 is 0. In certain embodiments, b9 is 1. In certain embodiments, b9 is 2. In certain embodiments, b9 is 3.

As generally defined herein, b10 is independently 0 or an integer of 1 to 10, inclusive. In certain embodiments, b10 is 0. In certain embodiments, b10 is 1. In certain embodiments, b10 is 2. In certain embodiments, b10 is 3. In certain embodiments, b10 is 4.

As generally defined herein, b11 is independently 0, 1, 2, 3, 4, 5, 6, 7, or 8. As generally defined herein, b11 is independently 0, 1, 2, 3, 4, 5, 6, 7, 8, 9, or 10. In certain embodiments, b11 is 0. In certain embodiments, b11 is 1. In certain embodiments, b11 is 2. In certain embodiments, b11 is 3. In certain embodiments, b11 is 4. In certain embodiments, b11 is 5. In certain embodiments, b11 is 6. In certain embodiments, b11 is 7. In certain embodiments, b11 is 8. In certain embodiments, b11 is 9. In certain embodiments, b11 is 10.

In certain embodiments, B is selected from the group consisting of:

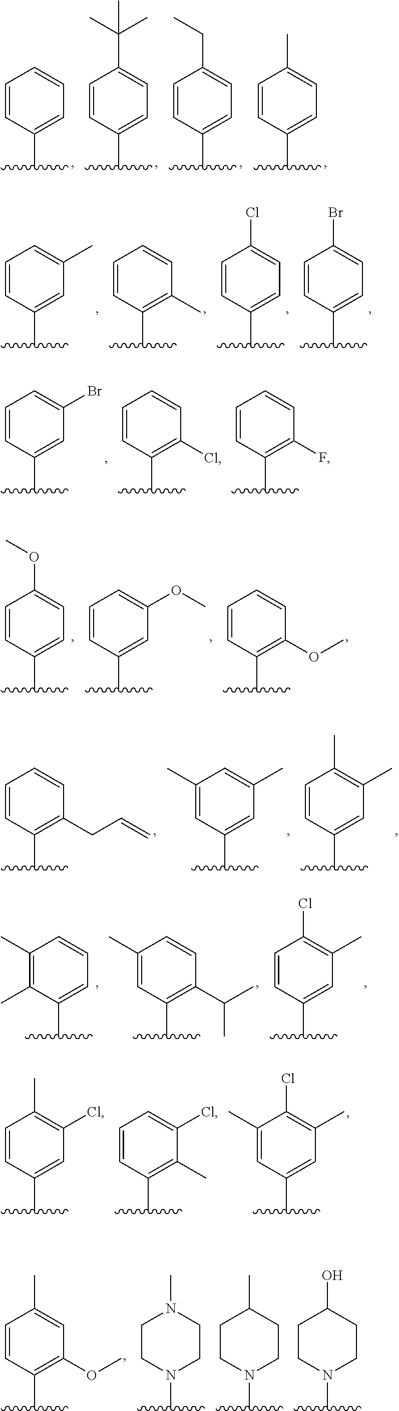

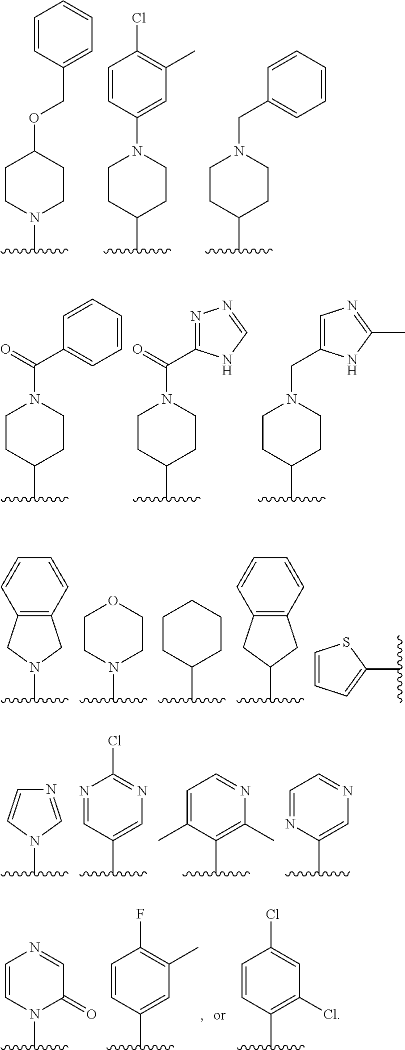

In certain embodiments, B is selected from the group consisting of:

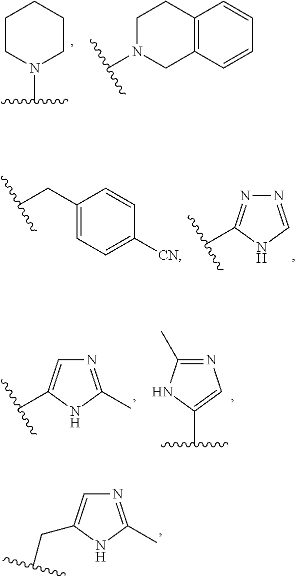

In certain embodiments, B is

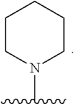 [SMILES: *N1CCCCC1]

In certain embodiments, B is

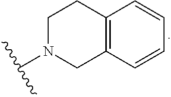 [SMILES: *N1CCc2ccccc2C1]

In certain embodiments, B is

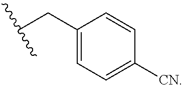 [SMILES: *Cc1ccc(C#N)cc1]

In certain embodiments, B is

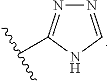 [SMILES: *c1nnc[nH]1]

In certain embodiments, B is

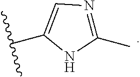 [SMILES: *c1cnc(C)[nH]1]

In certain embodiments, B is

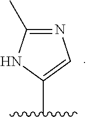 [SMILES: *c1cnc(C)[nH]1]

In certain embodiments, B is

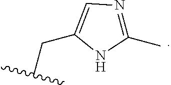 [SMILES: *Cc1cnc(C)[nH]1]

In certain embodiments, B is selected from the group consisting of:

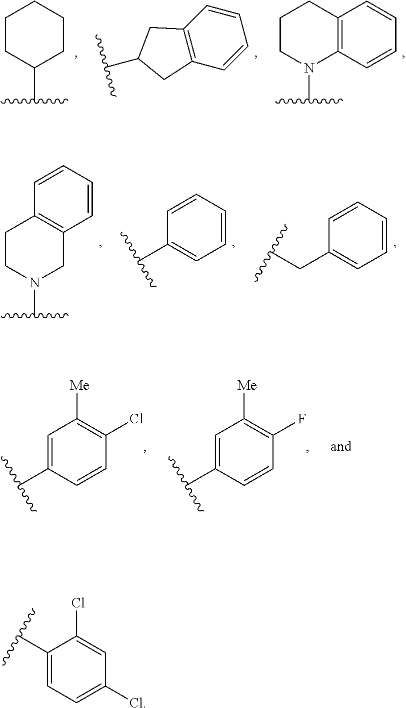

In certain embodiments, B is

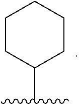 [SMILES: *C1CCCCC1]

In certain embodiments, B is

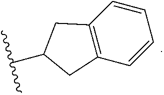 [SMILES: *C1Cc2ccccc2C1]

In certain embodiments, B is

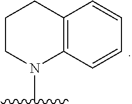 [SMILES: *N1CCCc2ccccc21]

In certain embodiments, B is

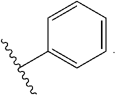 [SMILES: *c1ccccc1]

In certain embodiments, B is

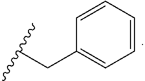 [SMILES: *Cc1ccccc1]

In certain embodiments, B is

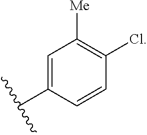 [SMILES: *c1ccc(Cl)c(C)c1]

In certain embodiments, B is

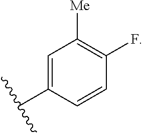 [SMILES: *c1ccc(F)c(C)c1]

In certain embodiments, B is

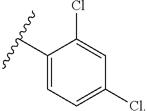 [SMILES: *c1ccc(Cl)cc1Cl]

As generally defined herein, in Formula (I′), or Formula (I)-(VII), L is a divalent linker. In certain embodiments, L is a bond, —O—, —C(═O)—, —NR^{LB}C(═O)—, —C(═O)NR^{LB}—, —NR^{LB}—, or —SO₂—, wherein R^{LB} is as defined herein. In certain embodiments, L is a bond. In certain embodiments, L is —O—. In certain embodiments, L is —C(═O)—. In certain embodiments, L is —NR^{LB}C(═O)—. In certain embodiments, L is —C(═O)NR^{LB}—. In certain embodiments, L is —NR^{LB}—. In certain embodiments, L is —NH—. In certain embodiments, L is —SO₂—. As used herein, each instance of R^{LB} is independently hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group. In certain embodiments, R^{LB} is hydrogen. In certain embodiments, R^{LB} is optionally substituted alkyl. In certain embodiments, R^{LB} is optionally substituted C₁₋₆ alkyl. In certain embodiments, R^{LB} is methyl. In certain embodiments, R^{LB} is a nitrogen protecting group. In certain embodiments, R^{LB} is acetyl, Bn, BOC, Cbz, Fmoc, or Ts. In certain embodiments, R^{LB} and B are taken together with their intervening atoms to form an optionally substituted heterocyclic ring.

In certain embodiments, L is —NR^{LB} C(═O)—, wherein R^{LB} is hydrogen, optionally substituted C₁₋₆ alkyl, optionally substituted aryl, or a nitrogen protecting group. In certain embodiments, L is —NHC(═O)—. In certain embodiments, L is —NR^{LB} C(═O)—, wherein R^{LB} is unsubstituted C₁₋₆ alkyl. In certain embodiments, L is —NR^{LB} C(═O)—, wherein R^{LB} is methyl, ethyl, or propyl. In certain embodiments, L is —NR^{LB} C(═O)—, wherein R^{LB} is optionally substituted aryl. In certain embodiments, L is —NR^{LB} C(═O)—, wherein R^{LB} is phenyl. In certain embodiments, L is —NR^{LB} C(═O)—, wherein R^{LB} is 4-methylphenyl, 3-methylphenyl, or 2-methylphenyl. In certain embodiments, L is —NR^{LB}C(═O)—, wherein R^{LB} is 2,3-dimethylphenyl, 2,4-dimethylphenyl, 2,5-dimethylphenyl, 2,6-dimethylphenyl, 3,4-dimethylphenyl, 3,5-dimethylphenyl, or 3,6-dimethylphenyl. In certain embodiments, L is —NR^{LB}C(═O)—, wherein R^{LB} is a nitrogen protecting group. In certain embodiments, L is —NR^{LB}C(═O)—, wherein R^{LB} is acetyl, Bn, BOC, Cbz, Fmoc, or Ts.

In certain embodiments, L is —C(═O)NR^{LB}—, wherein R^{LB} is hydrogen, optionally substituted C₁₋₆ alkyl, optionally substituted aryl, or a nitrogen protecting group. In certain embodiments, L is —C(═O)NH—. In certain embodiments, L is —C(═O)NR^{LB}—, wherein R^{LB} is unsubstituted C₁₋₆ alkyl. In certain embodiments, L is —C(═O)NR^{LB}—, wherein R^{LB} is methyl, ethyl, or propyl. In certain embodiments, L is —C(═O)NR^{LB}—, wherein R^{LB} is optionally substituted aryl. In certain embodiments, L is —C(═O)NR^{LB}—, wherein R^{LB} is phenyl. In certain embodiments, L is —C(═O)NR^{LB}—, wherein R^{LB} is 4-methylphenyl, 3-methylphenyl, or 2-methylphenyl. In certain embodiments, L is —C(═O)NR^{LB}—, wherein R^{LB} is 2,3-dimethylphenyl, 2,4-dimethylphenyl, 2,5-dimethylphenyl, 2,6-dimethylphenyl, 3,4-dimethylphenyl, 3,5-dimethylphenyl, or 3,6-dimethylphenyl. In certain embodiments, L is —C(═O)NR^{LB}—, wherein R^{LB} is a nitrogen protecting group. In certain embodiments, L is —C(═O)NR^{LB}—, wherein R^{LB} is acetyl, Bn, BOC, Cbz, Fmoc, or Ts.

In certain embodiments, L is —NR^{LB}—, wherein R^{LB} is hydrogen, optionally substituted C₁₋₆ alkyl, optionally substituted aryl, or a nitrogen protecting group. In certain embodiments, L is —NH—. In certain embodiments, L is —NR^{LB}—, wherein R^{LB} is unsubstituted C₁₋₆ alkyl. In certain embodiments, L is —NR^{LB}—, wherein R^{LB} is methyl, ethyl, or propyl. In certain embodiments, L is —NR^{LB}—, wherein R^{LB} is optionally substituted aryl.

In certain embodiments, L is —NR^{LB}—, wherein R^{LB} is phenyl. In certain embodiments, L is —NR^{LB}—, wherein R^{LB} is 4-methylphenyl, 3-methylphenyl, or 2-methylphenyl. In certain embodiments, L is —NR^{LB}—, wherein R^{LB} is 2,3-dimethylphenyl, 2,4-dimethylphenyl, 2,5-dimethylphenyl, 2,6-dimethylphenyl, 3,4-dimethylphenyl, 3,5-dimethylphenyl, or 3,6-dimethylphenyl. In certain embodiments, L is —NR^{LB}—, wherein R^{LB} is a nitrogen protecting group. In certain embodiments, L is —NR^{LB}—, wherein R^{LB} is acetyl, Bn, BOC, Cbz, Fmoc, or Ts.

As generally defined herein, in Formula (I′) or Formula (I), Y is a bond, optionally substituted C₁₋₆ alkylene, optionally substituted C₁₋₆ carbocyclylene, or optionally substituted heterocyclylene. In certain embodiments, Y is a bond. In certain embodiments, Y is optionally substituted C₁₋₆ alkylene. In certain embodiments, Y is unsubstituted C₁₋₆ alkylene. In certain embodiments, Y is unsubstituted straight chain C₁₋₆ alkylene. In certain embodiments, Y is substituted straight chain C₁₋₆alkylene. In certain embodiments, Y is unsubstituted branched C₁₋₆ alkylene. In certain embodiments, Y is substituted branched C₁₋₆ alkylene.

In certain embodiments, Y is of the formula

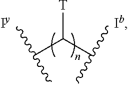

wherein l^{y} indicates the point of attachment to X, l^{b} indicates the point of attachment to B; n is 0, 1, 2, 3, 4, 5, or 6; each instance of T is independently hydrogen, halogen, optionally substituted C₁₋₆ alkyl, or —OR^{T}; and each instance of R^{T} is independently hydrogen, optionally substituted C₁₋₆ alkyl, or an oxygen protecting group. In certain embodiments, n is 0. In certain embodiments, n is 1. In certain embodiments, n is 2. In certain embodiments, n is 3. In certain embodiments, n is 4. In certain embodiments, n is 5. In certain embodiments, n is 6. In certain embodiments, at least one instance of T is hydrogen. In certain embodiments, all instances of T are hydrogen. In certain embodiments, at least one instance of T is halogen. In certain embodiments, at least two instances of T are halogen. In certain embodiments, one instance of T is halogen. In certain embodiments, one instance of T is F. In certain embodiments, at least two instances of T are F. In certain embodiments, one instance of T is Cl. In certain embodiments, one instance of T is Br. In certain embodiments, one instance of T is I. In certain embodiments, one instance of T is optionally substituted C₁₋₆ alkyl. In certain embodiments, one instance of T is unsubstituted C₁₋₆ alkyl. In certain embodiments, one instance of T is methyl. In certain embodiments, one instance of T is —OR^{T}, where R^{T} is as defined herein. In certain embodiments, one instance of T is —OH.

In certain embodiments, Y is of the formula

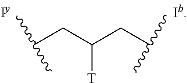

In certain embodiments, Y is of the formula

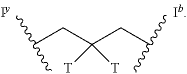

In certain embodiments, Y is of the formula

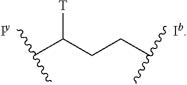

In certain embodiments, Y is of the formula

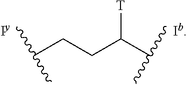

In certain embodiments, Y is of the formula

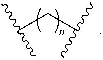

In certain embodiments, Y is of one of the following formulae:

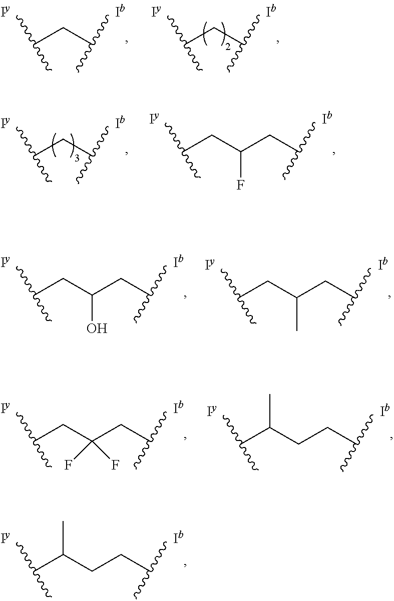

In some embodiments, when A is optionally substituted phenyl or unsubstituted 5-membered heteroaryl, X is not —S—. In some embodiments, when A is phenyl or mono-substituted phenyl, X is not —S—. In some embodiments, when A is unsubstituted furan or unsubstituted thiophene, X is not —S—. In some embodiments, when A is monosubsituted phenyl having one substituent selected from the group consisting of halogen, C₁₋₃ alkyl, and C₁₋₃ alkoxy, X is not —S—.

In some embodiments, when A is optionally substituted phenyl or unsubstituted 5-membered heteroaryl, and X is —S—, L is —O— or —C(═O)—. In some embodiments, when A is phenyl or mono-substituted phenyl, and X is —S—, L is —O— or —C(═O)—. In some embodiments, when A is unsubstituted furanyl or unsubstituted thiophenyl, and X is —S—, L is —O— or —C(═O)—. In some embodiments, when A is phenyl having a mono-substituent selected from the group consisting of halogen, C₁₋₃ alkyl, and C₁₋₃ alkoxy, and X is —S—, L is —O— or —C(═O)—.

In some embodiments, when A is optionally substituted phenyl or unsubstituted 5-membered heteroaryl, and X is —S—, B is not optionally substituted phenyl or optionally substituted bicyclic heteroaryl. In some embodiments, when A is phenyl or mono-substituted phenyl, and X is —S—, B is not mono-substituted or di-substituted phenyl. In some embodiments, when A is unsubstituted furan or unsubstituted thiophene, and X is —S—, B is not a phenyl ring mono-substituted or di-substituted by halogen, C₁₋₃ alkyl, or C₁₋₃ alkoxy.

In some embodiments, when A is optionally substituted phenyl or unsubstituted 5-membered heteroaryl; X is —S—; and L is a bond, B is not optionally substituted phenyl or optionally substituted 5-membered heteroaryl. In some embodiments, when A is optionally substituted phenyl or unsubstituted 5-membered heteroaryl; X is —S—; and L is —O—, B is not optionally substituted phenyl or optionally substituted 5-membered heteroaryl. In some embodiments, when A is optionally substituted phenyl or unsubstituted 5-membered heteroaryl; X is —S—; and L is —NR^{LB}C(═O)—, B is not optionally substituted C₁₋₆ alkyl, optionally substituted phenyl, or optionally substituted furan or optionally substituted thiophene. In some embodiments, when A is optionally substituted phenyl, unsubstituted furan or unsubstituted thiophene; X is —S—; and L is —NR^{LB} C(═O)—, R^{LB} and B are not taken together with their intervening atoms to form an optionally substituted heterocyclic ring.

In certain embodiments, X is —NR^{NX}—, Y is a bond, and L is a bond. In certain embodiments, X is —NH—, Y is a bond, and L is a bond. In certain embodiments, X is —NR^{NX}—, Y is optionally substituted C₁₋₆ alkylene, and L is —O—. In certain embodiments, X is —NH—, Y is optionally substituted C₁₋₃ alkylene, and L is —O—.

In certain embodiments, X is —NR^{NX}—, Y is optionally substituted C₁₋₆ alkylene, and L is a bond. In certain embodiments, X is —NH—, Y is optionally substituted C₁₋₃ alkylene, and L is a bond. In certain embodiments, X is —NR^{NX}—, Y is optionally substituted C₁₋₆ alkylene, and L is —NR^{LB}—. In certain embodiments, X is —NH—, Y is optionally substituted C₁₋₃ alkylene, and L is —NR^{LB}—. In certain embodiments, X is —NR^{NX}—, Y is optionally substituted C₁₋₆ alkylene, and L is —SO₂—. In certain embodiments, X is —NH—, Y is optionally substituted C₁₋₃ alkylene, and L is —SO₂—. In certain embodiments, X is —NR^{NX}—, Y is optionally substituted C₁₋₆ alkylene, and L is a bond. In certain embodiments, X is —NH—, Y is optionally substituted C₁₋₃ alkylene, and L is a bond. In certain embodiments, X is —NR^{NX}—, Y is optionally substituted carbocyclylene, and L is a bond. In certain embodiments, X is —NH—, Y is optionally substituted carbocyclylene, and L is a bond. In certain embodiments, X is —NR^{NX}—, Y is optionally substituted carbocyclylene, and L is —NR^{LB}—. In certain embodiments, X is —NH—, Y is optionally substituted carbocyclylene, and L is —NR^{LB}—.

In certain embodiments, X is —NR^{NX}—, Y is optionally substituted heterocyclylene, and L is a bond. In certain embodiments, X is —NH—, Y is optionally substituted heterocyclylene, and L is a bond. In certain embodiments, X is —NR^{NX}—, Y is optionally substituted heterocyclylene, and L is —C(═O)—. In certain embodiments, X is —NH—, Y is optionally substituted heterocyclylene, and L is —C(═O)—.

In certain embodiments, X is O, Y is a bond, and L is a bond. In certain embodiments, X is S, Y is a bond, and L is a bond. In certain embodiments, X is optionally substituted heterocyclene, Y is a bond, and L is a bond. In certain embodiments, X is optionally substituted heterocyclene, Y is optionally substituted C₁₋₆ alkylene, and L is —NR^{LB}—. In certain embodiments, X is optionally substituted heterocyclene, Y is optionally substituted C₁₋₃ alkylene, and L is —NR^{LB}—. In certain embodiments, X is optionally substituted heterocyclene, Y is optionally substituted C₁₋₆ alkylene, and L is —O—. In certain embodiments, X is optionally substituted heterocyclene, Y is optionally substituted C₁₋₃ alkylene, and L is —O—. In certain embodiments, X is optionally substituted heterocyclene, Y is a optionally substituted C₁₋₆ alkylene, and L is a bond. In certain embodiments, X is optionally substituted heterocyclene, Y is a optionally substituted C₁₋₃ alkylene, and L is a bond. In certain embodiments, X is optionally substituted heterocyclene, Y is a bond, and L is —O—.

In certain embodiments, X is —NR^{NX}—, Y is a bond, L is a bond, and B is hydrogen. In certain embodiments, X is —NH—, Y is a bond, L is a bond, and B is hydrogen. In certain embodiments, X is —NH—, Y is optionally substituted C₁₋₆ alkylene, L is —O—, and B is hydrogen. In certain embodiments, X is —NR^{NX}—, Y is optionally substituted C₁₋₆ alkylene, L is —O—, and B is hydrogen. In certain embodiments, X is —NR^{NX}—, Y is optionally substituted C₁₋₃ alkylene, L is —O—, and B is hydrogen.

In certain embodiments, X is —NR^{NX}—, Y is optionally substituted C₁₋₆ alkylene, L is —O—, and B is optionally substituted alkyl. In certain embodiments, X is —NH—, Y is optionally substituted C₁₋₃ alkylene, L is —O—, and B is optionally substituted alkyl. In certain embodiments, X is —NR^{NX}—, Y is optionally substituted C₁₋₆ alkylene, L is —O—, and B is optionally substituted carbocyclyl. In certain embodiments, X is —NH—, Y is optionally substituted C₁₋₃ alkylene, L is —O—, and B is optionally substituted carbocyclyl. In certain embodiments, X is —NR^{NX}—, Y is optionally substituted C₁₋₆ alkylene, L is —O—, and B is optionally substituted aryl. In certain embodiments, X is —NH—, Y is optionally substituted C₁₋₃ alkylene, L is —O—, and B is optionally substituted aryl. In certain embodiments, X is —NR^{NX}—, Y is optionally substituted C₁₋₆ alkylene, L is —O—, and B is optionally substituted heteroaryl. In certain embodiments, X is —NH—, Y is optionally substituted C₁₋₃ alkylene, L is —O—, and B is optionally substituted heteroaryl.

In certain embodiments, X is —NR^{NX}—, Y is optionally substituted C₁₋₆ alkylene, L is —NR^{LB}—, and R^{LB} and B are taken together with their intervening atoms to form an optionally substituted heterocyclic ring. In certain embodiments, X is —NH—, Y is optionally substituted C₁₋₃ alkylene, L is —NR^{LB}—, and R^{LB} and B are taken together with their intervening atoms to form an optionally substituted heterocyclic ring.

In certain embodiments, X is —NR^{NX}—, Y is optionally substituted C₁₋₆ alkylene, L is —NR^{LB}—, and B is optionally substituted alkyl. In certain embodiments, X is —NH—, Y is optionally substituted C₁₋₃ alkylene, L is —NR^{LB}—, and B is optionally substituted alkyl. In certain embodiments, X is —NR^{NX}—, Y is optionally substituted C₁₋₆ alkylene, L is —NR^{LB}—, and B is optionally substituted carbocyclyl. In certain embodiments, X is —NH—, Y is optionally substituted C₁₋₃ alkylene, L is —NR^{LB}—, and B is optionally substituted carbocyclyl. In certain embodiments, X is —NR^{NX}—, Y is optionally substituted C₁₋₆ alkylene, L is —NR^{LB}—, and B is optionally substituted aryl. In certain embodiments, X is —NH—, Y is optionally substituted C₁₋₃ alkylene, L is —NR^{LB}—, and B is optionally substituted aryl.

In certain embodiments, X is —NR^{NX}—, Y is optionally substituted C₁₋₆ alkylene, L is —SO₂—, and B is optionally substituted aryl. In certain embodiments, X is —NH—, Y is optionally substituted C₁₋₃ alkylene, L is —SO₂—, and B is optionally substituted aryl. In certain embodiments, X is —NR^{NX}—, Y is optionally substituted C₁₋₆ alkylene, L is a bond, and B is hydrogen. In certain embodiments, X is —NH—, Y is optionally substituted C₁₋₃ alkylene, L is a bond, and B is hydrogen. In certain embodiments, —NR^{NX}—, Y is optionally substituted C₁₋₆ alkylene, L is a bond, and B is optionally substituted alkyl. In certain embodiments, —NH—, Y is optionally substituted C₁₋₃ alkylene, L is a bond, and B is optionally substituted alkyl. In certain embodiments, X is —NR^{NX}—, Y is optionally substituted C₁₋₆ alkylene, L is a bond, and B is optionally substituted heterocyclyl. In certain embodiments, X is —NH—, Y is optionally substituted C₁₋₃ alkylene, L is a bond, and B is optionally substituted heterocyclyl. In certain embodiments, X is —NR^{NX}—, Y is optionally substituted C₁₋₆ alkylene, L is a bond, and B is optionally substituted aryl. In certain embodiments, X is —NH—, Y is optionally substituted C₁₋₃ alkylene, L is a bond, and B is optionally substituted aryl. In certain embodiments, X is —NR^{NX}—, Y is optionally substituted C₁₋₆ alkylene, L is a bond, and B is optionally substituted heteroaryl. In certain embodiments, X is —NH—, Y is optionally substituted C₁₋₃ alkylene, L is a bond, and B is optionally substituted heteroaryl.

In certain embodiments, X is —NR^{NX}—, Y is optionally substituted carbocyclylene, L is a bond, and B is optionally substituted alkyl. In certain embodiments, X is —NH—, Y is optionally substituted carbocyclylene, L is a bond, and B is optionally substituted alkyl. In certain embodiments, X is —NR^{NX}—, Y is optionally substituted carbocyclylene, L is —NR^{LB}—, and B is optionally substituted alkyl. In certain embodiments, X is —NH—, Y is optionally substituted carbocyclylene, L is —NR^{LB}—, and B is optionally substituted alkyl.

In certain embodiments, X is —NR^{NX}—, Y is optionally substituted heterocyclylene, L is a bond, and B is optionally substituted alkyl. In certain embodiments, X is —NH—, Y is optionally substituted heterocyclylene, L is a bond, and B is optionally substituted alkyl. In certain embodiments, X is —NR^{NX}—, Y is optionally substituted heterocyclylene, L is a bond, and B is optionally substituted aryl. In certain embodiments, X is —NH—, Y is optionally substituted heterocyclylene, L is a bond, and B is optionally substituted aryl. In certain embodiments, X is —NR^{NX}—, Y is optionally substituted heterocyclylene, L is a bond, and B is optionally substituted heteroaryl. In certain embodiments, X is —NH—, Y is optionally substituted heterocyclylene, L is a bond, and B is optionally substituted heteroaryl.

In certain embodiments, X is —NR^{NX}—, Y is optionally substituted heterocyclylene, L is —C(═O)—, and B is optionally substituted heteroaryl. In certain embodiments, X is —NH—, Y is optionally substituted heterocyclylene, L is —C(═O)—, and B is optionally substituted heteroaryl. In certain embodiments, X is —NR^{NX}—, Y is optionally substituted heterocyclylene, L is —C(═O)—, and B is aryl. In certain embodiments, X is —NH—, Y is optionally substituted heterocyclylene, L is —C(═O)—, and B is aryl.

In certain embodiments, X is O, Y is a bond, L is a bond, and B is hydrogen. In certain embodiments, X is S, Y is a bond, L is a bond, and B is hydrogen. In certain embodiments, X is optionally substituted heterocyclene, Y is a bond, L is a bond, and B is hydrogen. In certain embodiments, X is optionally substituted heterocyclene, Y is a bond, L is a bond, and B is optionally substituted aryl. In certain embodiments, X is optionally substituted heterocyclene, Y is a bond, L is a bond, and B is optionally substituted aryl. In certain embodiments, X is optionally substituted heterocyclene, Y is a bond, L is —NR^{LB}—, and B is hydrogen or optionally substituted aryl. In certain embodiments, X is optionally substituted heterocyclene, Y is a bond, L is a bond, and B is optionally substituted aryl. In certain embodiments, X is optionally substituted heterocyclene, Y is a bond, L is —NR^{LB}—, and B is optionally substituted aryl. In certain embodiments, X is optionally substituted heterocyclene, Y is a bond, L is —NH—, and B is hydrogen or optionally substituted aryl. In certain embodiments, X is optionally substituted heterocyclene, Y is optionally substituted C₁₋₆ alkylene, L is —NR^{LB}—, and B is optionally substituted alkyl. In certain embodiments, X is optionally substituted heterocyclene, Y is optionally substituted C₁₋₆ alkylene, L is —NR^{LB}—, and B is optionally substituted aryl. In certain embodiments, X is optionally substituted heterocyclene, Y is optionally substituted C₁₋₃ alkylene, L is —NR^{LB}—, and B is optionally substituted alkyl. In certain embodiments, X is optionally substituted heterocyclene, Y is optionally substituted C₁₋₆ alkylene, L is —O—, and B is hydrogen. In certain embodiments, X is optionally substituted heterocyclene, Y is optionally substituted C₁₋₆ alkylene, L is —O—, and B is optionally substituted alkyl. In certain embodiments, X is optionally substituted heterocyclene, Y is optionally substituted C₁₋₃ alkylene, L is —O—, and B is optionally substituted alkyl. In certain embodiments, X is optionally substituted heterocyclene, Y is a optionally substituted C₁₋₆ alkylene, L is a bond, and B is hydrogen. In certain embodiments, X is optionally substituted heterocyclene, Y is a optionally substituted C₁₋₃ alkylene, L is a bond, and B is hydrogen. In certain embodiments, X is optionally substituted heterocyclene, Y is a bond, L is —O—, and B is hydrogen. In certain embodiments, X is optionally substituted heterocyclene, Y is a bond, L is —O—, and B is optionally substituted alkyl.

Formula (IA) includes Z. In certain embodiments, Z is S. In certain embodiments, Z is O. In certain embodiments, Formula (IA) is not of the formula:

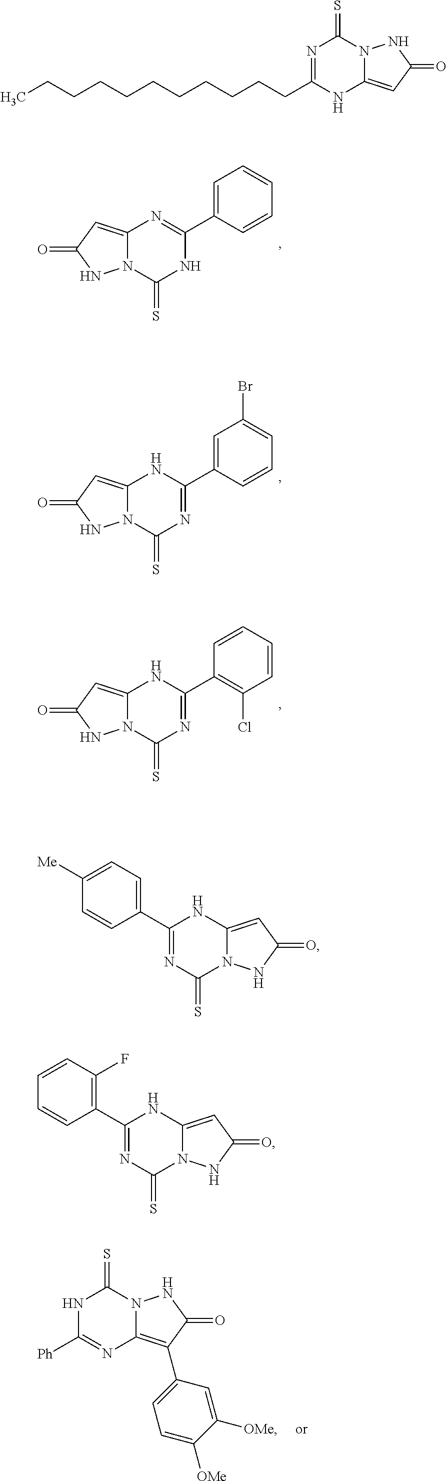

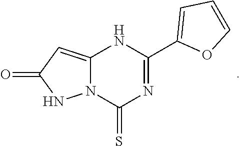 [SMILES: O=c1cc2[nH]c(-c3ccco3)nc(=S)n2[nH]1]

In certain embodiments, a provided compound is of Formula (I):

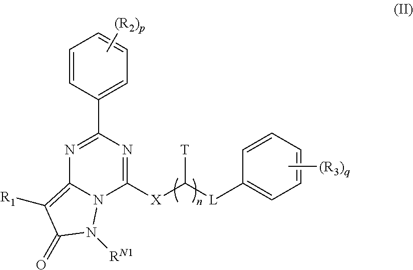

or a pharmaceutically acceptable salt, solvate, hydrate, polymorph, co-crystal, tautomer, stereoisomer, isotopically labeled derivative, or prodrug thereof, wherein R^{N1}, R₂, R₃, T, L, X, p, q, and n are as described herein.

In certain embodiments, a provided compound is of Formula (II-i):

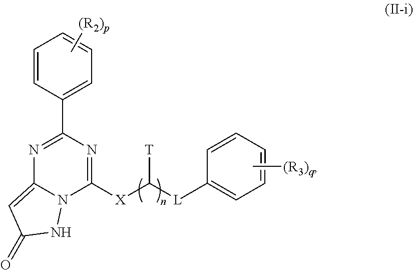

or a pharmaceutically acceptable salt, solvate, hydrate, polymorph, co-crystal, tautomer, stereoisomer, isotopically labeled derivative, or prodrug thereof, wherein R₂, R₃, T, L, X, p, q, and n are as described herein.

In certain embodiments, in Formula (I′), Formula (IA), or Formulae (I)-(VII), each instance of R₂ is independently selected from the group consisting of hydrogen, halogen, —CN, —NO₂, —N₃, unsubstituted alkyl, unsubstituted alkenyl, unsubstituted alkynyl, unsubstituted carbocyclyl, unsubstituted aryl, unsubstituted heterocyclyl, unsubstituted heteroaryl, —OR^{A}, —N(R^{B})₂, —SR^{A}, —C(═O)R^{A}, —C(═O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, and —SO₂N(R^{B})₂; each instance of R₃ is independently selected from the group consisting of hydrogen, halogen, —CN, —NO₂, —N₃, unsubstituted alkyl, unsubstituted alkenyl, unsubstituted alkynyl, unsubstituted carbocyclyl, unsubstituted aryl, unsubstituted heterocyclyl, unsubstituted heteroaryl, —OR^{A}, —N(R^{B})₂, —SR^{A}, —C(═O)R^{A}, —C(O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, and —SO₂N(R^{B})₂; each instance of R^{A} is independently selected from the group consisting of hydrogen, unsubstituted alkyl, unsubstituted alkenyl, unsubstituted alkynyl, unsubstituted carbocyclyl, unsubstituted heterocyclyl, unsubstituted aryl, unsubstituted heteroaryl, and an oxygen protecting group; each instance of R^{B} is independently selected from the group consisting of hydrogen, unsubstituted alkyl, unsubstituted alkenyl, unsubstituted alkynyl, unsubstituted carbocyclyl, unsubstituted heterocyclyl, unsubstituted aryl, unsubstituted heteroaryl, and a nitrogen protecting group, or two R^{B} groups are taken together with their intervening atoms to form an unsubstituted heterocyclic ring. In certain embodiments, each instance of R₂ is independently selected from the group consisting of hydrogen, halogen, —CN, —NO₂, —N₃, unsubstituted alkyl, unsubstituted alkenyl, unsubstituted alkynyl, unsubstituted carbocyclyl, unsubstituted aryl, unsubstituted heterocyclyl, unsubstituted heteroaryl, —OR^{A}, —N(R^{B})₂, —SR^{A}, and —C(═O)R^{A}. In certain embodiments, each instance of R₃ is independently selected from the group consisting of hydrogen, halogen, —CN, —NO₂, —N₃, unsubstituted alkyl, unsubstituted alkenyl, unsubstituted alkynyl, unsubstituted carbocyclyl, unsubstituted aryl, unsubstituted heterocyclyl, unsubstituted heteroaryl, —OR^{A}, —N(R^{B})₂, —SR^{A}, and —C(═O)R^{A}.

In certain embodiments, a provided compound is of Formula (II-a):

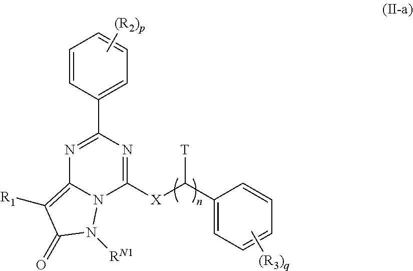

or a pharmaceutically acceptable salt, solvate, hydrate, polymorph, co-crystal, tautomer, stereoisomer, isotopically labeled derivative, or prodrug thereof, wherein R^{N1}, R₁, R₂, R₃, X, T, p, q, and n are as described herein.

In certain embodiments, a provided compound is of Formula (II-b):

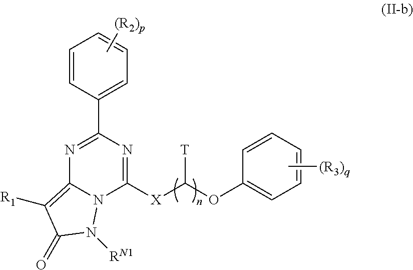

or a pharmaceutically acceptable salt, solvate, hydrate, polymorph, co-crystal, tautomer, stereoisomer, isotopically labeled derivative, or prodrug thereof, wherein R^{N1}, R₁, R₂, R₃, X, T, p, q, and n are as described herein.

In certain embodiments, a provided compound is of Formula (II-c):

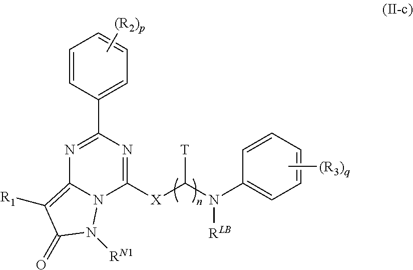

or a pharmaceutically acceptable salt, solvate, hydrate, polymorph, co-crystal, tautomer, stereoisomer, isotopically labeled derivative, or prodrug thereof, wherein R^{N1}, R¹, R₁, R₂, R₃, T, X, p, q, and n are as described herein.

In certain embodiments, a provided compound is of Formula (II-d):

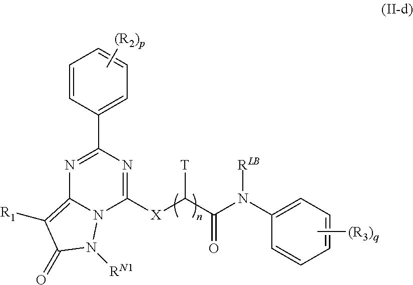

or a pharmaceutically acceptable salt, solvate, hydrate, polymorph, co-crystal, tautomer, stereoisomer, isotopically labeled derivative, or prodrug thereof, wherein R^{N1}, R^{LB}, R₁, R₂, R₃, T, X, p, q, and n are as described herein.

In certain embodiments, a provided compound is of Formula (II-e):

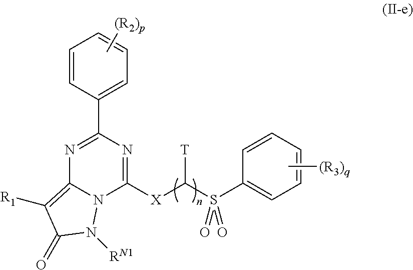

or a pharmaceutically acceptable salt, solvate, hydrate, polymorph, co-crystal, tautomer, stereoisomer, isotopically labeled derivative, or prodrug thereof, wherein R^{N1}, R₁, R₂, R₃, T, X, p, and q are as described herein.

In certain embodiments, a provided compound is of Formula (III):

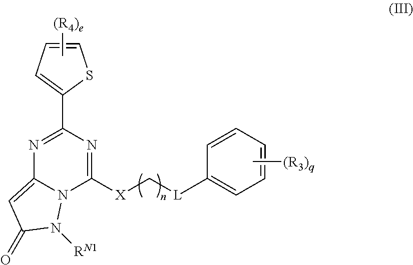

or a pharmaceutically acceptable salt, solvate, hydrate, polymorph, co-crystal, tautomer, stereoisomer, isotopically labeled derivative, or prodrug thereof, wherein R^{N1}, R₃, R₄, L, X, e, q, and n are as described herein.

In certain embodiments, a provided compound is of Formula (III-a):

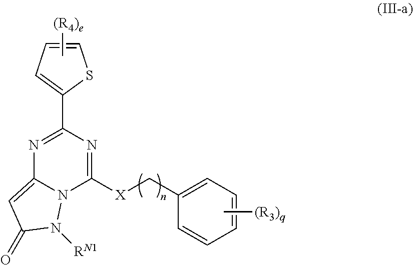

or a pharmaceutically acceptable salt, solvate, hydrate, polymorph, co-crystal, tautomer, stereoisomer, isotopically labeled derivative, or prodrug thereof, wherein R^{N1}, R₃, R₄, X, e, q, and n are as described herein.

In certain embodiments, a provided compound is of Formula (III-b):

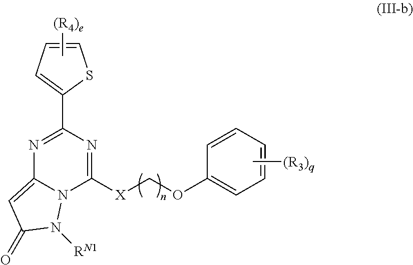

or a pharmaceutically acceptable salt, solvate, hydrate, polymorph, co-crystal, tautomer, stereoisomer, isotopically labeled derivative, or prodrug thereof, wherein R^{N1}, R₃, R₄, X, e, q, and n are as described herein.

In certain embodiments, a provided compound is of Formula (III-c):

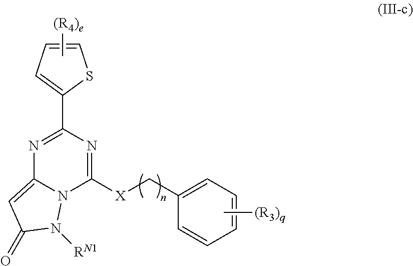

or a pharmaceutically acceptable salt, solvate, hydrate, polymorph, co-crystal, tautomer, stereoisomer, isotopically labeled derivative, or prodrug thereof, wherein R^{N1}, R₃, R₄, X, e, q, and n are as described herein.

In certain embodiments, a provided compound is of Formula (IV):

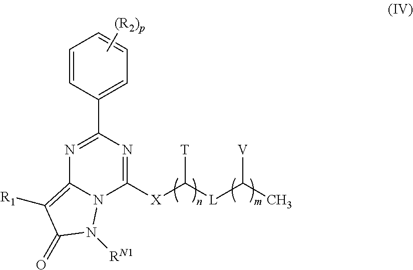

or a pharmaceutically acceptable salt, solvate, hydrate, polymorph, co-crystal, tautomer, stereoisomer, isotopically labeled derivative, or prodrug thereof, wherein R^{N1}, R₁, R₂, V, L, X, p, m, and n are as described herein.

In certain embodiments, a provided compound is of Formula (IV-i):

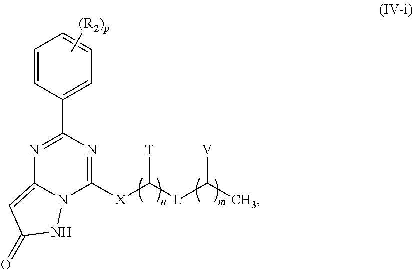

or a pharmaceutically acceptable salt, solvate, hydrate, polymorph, co-crystal, tautomer, stereoisomer, isotopically labeled derivative, or prodrug thereof, wherein R₂, X, T, V, L, p, m, and n are as described herein.

As generally defined herein, in Formula (IV), each instance of V is independently hydrogen, halogen, optionally substituted C₁₋₆ alkyl, optionally substituted alkenyl, optionally substituted alkynyl, optionally substituted carbocyclyl, optionally substituted aryl, optionally substituted heterocyclyl, optionally substituted heteroaryl, —N(R^{V1})₂, or —OR^{V2}; wherein R^{V1} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group, or two R^{V1} groups are taken together with their intervening atoms to form an optionally substituted heterocyclic ring; R^{V2} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and an oxygen protecting group. In certain embodiments, each instance of V is independently hydrogen, halogen, optionally substituted C₁₋₆ alkyl, or —OR^{V2}; and each instance of R^{V2} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and oxygen protecting groups. In certain embodiments, V is hydrogen. In certain embodiments, V is halogen (e.g., F, Cl, Br, or I). In certain embodiments, V is optionally substituted C₁₋₆ alkyl. In certain embodiments, V is unsubstituted C₁₋₆ alkyl (e.g., methyl). In certain embodiments, V is —OR^{v2}. In certain embodiments, V is —OH. In certain embodiments, V is —OR^{V}, wherein R^{V} is independently optionally substituted alkyl or an oxygen protecting group. In certain embodiments, V is —N(R^{V1})₂, wherein each instance of R^{V1} is independently hydrogen, optionally substituted alkyl, or a nitrogen protecting group. In certain embodiments, V is —NHR^{V1}, wherein R^{V1} is independently hydrogen, optionally substituted alkyl, or a nitrogen protecting group.

As generally defined herein, m is independently 0, 1, 2, 3, 4, 5, or 6. In some embodiments, m is 0. In some embodiments, m is 1. In some embodiments, m is 2. In some embodiments, m is 3. In some embodiments, m is 4. In some embodiments, m is 5. In some embodiments, m is 6.

In certain embodiments, a provided compound is of Formula (IV-a):

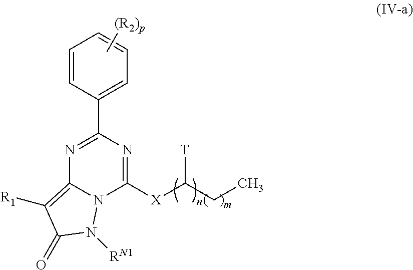

or a pharmaceutically acceptable salt, solvate, hydrate, polymorph, co-crystal, tautomer, stereoisomer, isotopically labeled derivative, or prodrug thereof, wherein R^{N1}, R₁, R₂, X, T, p, m, and n are as described herein.

In certain embodiments, a provided compound is of Formula (IV-b):

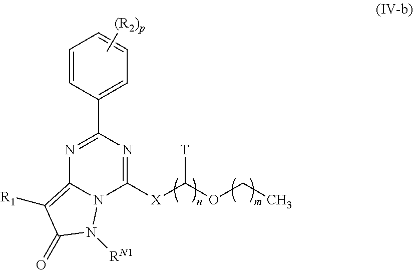

or a pharmaceutically acceptable salt, solvate, hydrate, polymorph, co-crystal, tautomer, stereoisomer, isotopically labeled derivative, or prodrug thereof, wherein R^{N1}, R₁, R₂, T, X, p, m, and n are as described herein.

In certain embodiments, a provided compound is of Formula (IV-c):

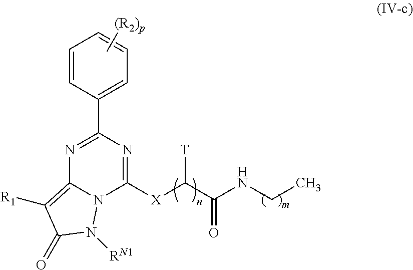

or a pharmaceutically acceptable salt, solvate, hydrate, polymorph, co-crystal, tautomer, stereoisomer, isotopically labeled derivative, or prodrug thereof, wherein R^{N1}, R₁, R₂, T, X, p, m, and n are as described herein.

In certain embodiments, a provided compound is of Formula (IV-d):

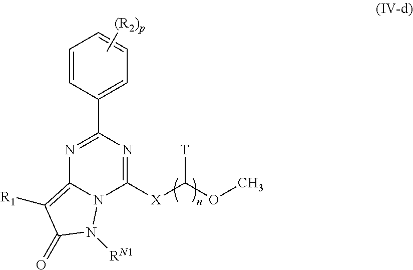

or a pharmaceutically acceptable salt, solvate, hydrate, polymorph, co-crystal, tautomer, stereoisomer, isotopically labeled derivative, or prodrug thereof, wherein R^{N1}, R₁, R₂, T, X, p, and n are as described herein.

In certain embodiments, a provided compound is of Formula (V):

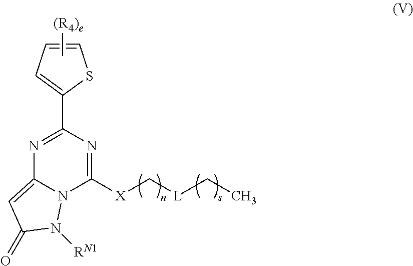

or a pharmaceutically acceptable salt, solvate, hydrate, polymorph, co-crystal, tautomer, stereoisomer, isotopically labeled derivative, or prodrug thereof, wherein R^{N1}, R₄, L, X, e, s, and n are as described herein.

In certain embodiments, a provided compound is of Formula (V-a):

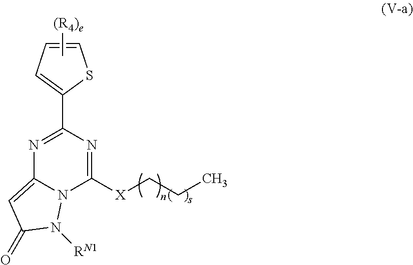

or a pharmaceutically acceptable salt, solvate, hydrate, polymorph, co-crystal, tautomer, stereoisomer, isotopically labeled derivative, or prodrug thereof, wherein R^{N1}, R₄, X, e, s, and n are as described herein.

In certain embodiments, a provided compound is of Formula (V-b):

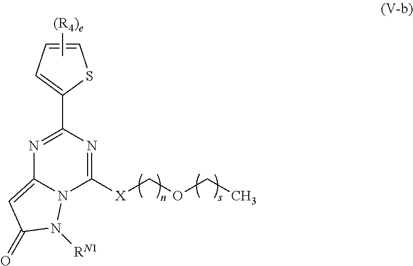

or a pharmaceutically acceptable salt, solvate, hydrate, polymorph, co-crystal, tautomer, stereoisomer, isotopically labeled derivative, or prodrug thereof, wherein R^{N1}, R₄, X, e, s, and n are as described herein.

In certain embodiments, a provided compound is of Formula (V-c):

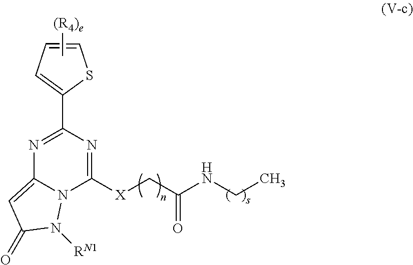

or a pharmaceutically acceptable salt, solvate, hydrate, polymorph, co-crystal, tautomer, stereoisomer, isotopically labeled derivative, or prodrug thereof, wherein R^{N1}, R₄, X, e, s, and n are as described herein.

As generally defined herein, in Formula (V), s is independently 0, 1, 2, 3, 4, 5, or 6. In some embodiments, s is 0. In some embodiments, s is 1. In some embodiments, s is 2. In some embodiments, s is 3. In some embodiments, s is 4. In some embodiments, s is 5. In some embodiments, s is 6.

In certain embodiments, a provided compound is of Formula (VI):

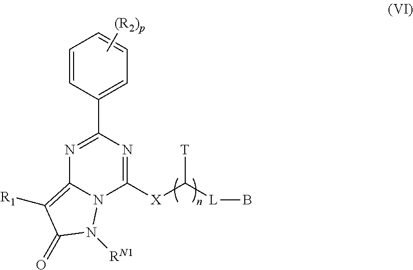

or a pharmaceutically acceptable salt, solvate, hydrate, polymorph, co-crystal, tautomer, stereoisomer, isotopically labeled derivative, or prodrug thereof, wherein R^{N1}, R₁, T, X, L, B, p, and n are as described herein.

In certain embodiments, a provided compound is Formula (VI-1-a):

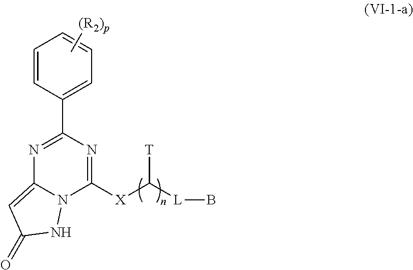

or a pharmaceutically acceptable salt, solvate, hydrate, polymorph, co-crystal, tautomer, stereoisomer, isotopically labeled derivative, or prodrug thereof, wherein R₂, T, X, L, B, p, and n are as described herein. In certain embodiments, a provided compound is of Formula (VI-a):

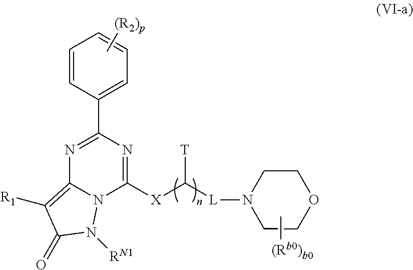

or a pharmaceutically acceptable salt, solvate, hydrate, polymorph, co-crystal, tautomer, stereoisomer, isotopically labeled derivative, or prodrug thereof, wherein R^{N1}, R^{b0}, R₁, R₂, T, X, L, n, p, and b0 are as described herein.

In certain embodiments, a provided compound is of Formula (VI-b):

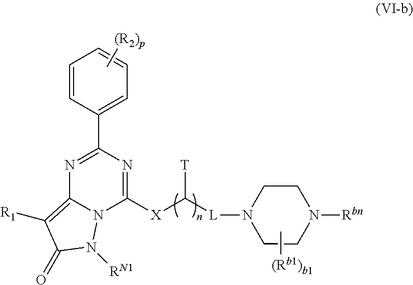

or a pharmaceutically acceptable salt, solvate, hydrate, polymorph, co-crystal, tautomer, stereoisomer, isotopically labeled derivative, or prodrug thereof, wherein R^{N1}, R^{b1}, R^{bn}, R₁, R₂, T, X, L, n, p, and b are as described herein.

In certain embodiments, a provided compound is of Formula (VI-c):

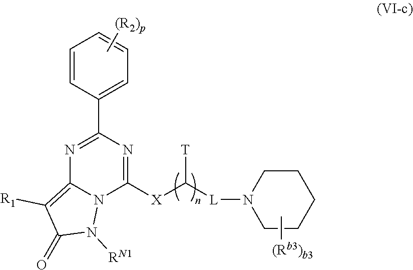

or a pharmaceutically acceptable salt, solvate, hydrate, polymorph, co-crystal, tautomer, stereoisomer, isotopically labeled derivative, or prodrug thereof, wherein R^{N1}, R^{b3}, R₁, R₂, T, X, L, n, p, and b3 are as described herein.

In certain embodiments, a provided compound is of Formula (VI-d):

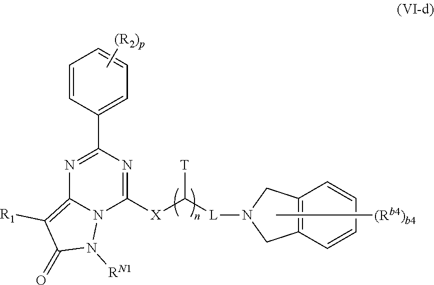

or a pharmaceutically acceptable salt, solvate, hydrate, polymorph, co-crystal, tautomer, stereoisomer, isotopically labeled derivative, or prodrug thereof, wherein R^{N1}, R^{b4}, R₁, R₂, T, X, L, n, p, and b4 are as described herein.

In certain embodiments, a provided compound is of Formula (VI-e):

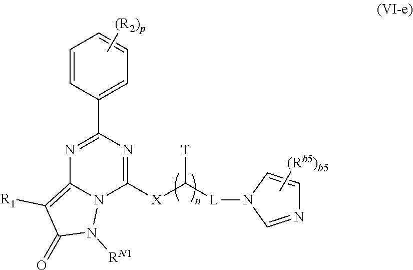

or a pharmaceutically acceptable salt, solvate, hydrate, polymorph, co-crystal, tautomer, stereoisomer, isotopically labeled derivative, or prodrug thereof, wherein R^{N1}, R^{b5}, R₁, T, X, L, n, p, and b5 are as described herein.

In certain embodiments, a provided compound is of Formula (VI-f):

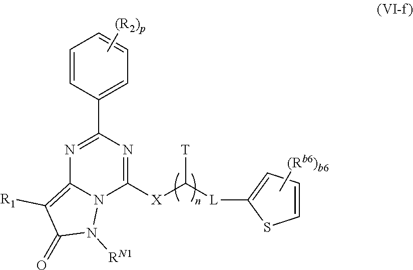

or a pharmaceutically acceptable salt, solvate, hydrate, polymorph, co-crystal, tautomer, stereoisomer, isotopically labeled derivative, or prodrug thereof, wherein R^{N1}, R^{b6}, R₁, R₂, T, X, L, n, p, and b6 are as described herein.

In certain embodiments, a provided compound is of Formula (VI-g):

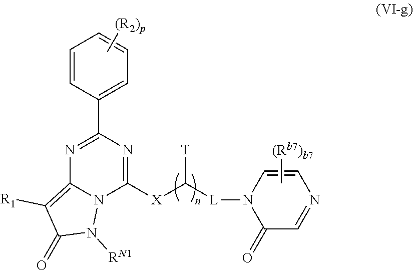

or a pharmaceutically acceptable salt, solvate, hydrate, polymorph, co-crystal, tautomer, stereoisomer, isotopically labeled derivative, or prodrug thereof, wherein R^{N1}, R^{b7}, R₁, R₂, T, X, L, n, p, and b7 are as described herein.

In certain embodiments, a provided compound is of Formula (VI-h):

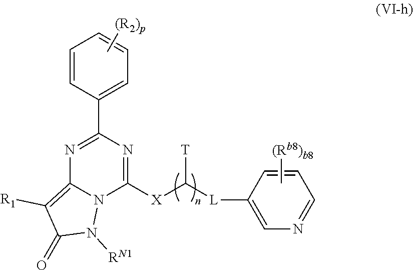

or a pharmaceutically acceptable salt, solvate, hydrate, polymorph, co-crystal, tautomer, stereoisomer, isotopically labeled derivative, or prodrug thereof, wherein R^{N1}, R^{b8}, R₁, R₂, T, X, L, n, p, and b8 are as described herein.

In certain embodiments, a provided compound is of Formula (VI-i):

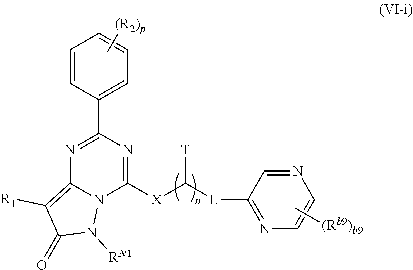

or a pharmaceutically acceptable salt, solvate, hydrate, polymorph, co-crystal, tautomer, stereoisomer, isotopically labeled derivative, or prodrug thereof, wherein R^{N1}, R^{b9}, R₁, R₂, T, X, L, n, p, and b9 are as described herein.

In certain embodiments, a provided compound is of Formula (VI-j):

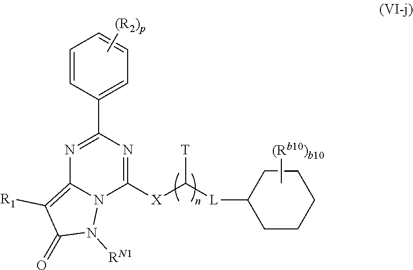

or a pharmaceutically acceptable salt, solvate, hydrate, polymorph, co-crystal, tautomer, stereoisomer, isotopically labeled derivative, or prodrug thereof, wherein R^{N1}, R^{b10}, R₁, R₂, T, X, L, n, p, and b10 are as described herein.

In certain embodiments, a provided compound is of Formula (VI-k):

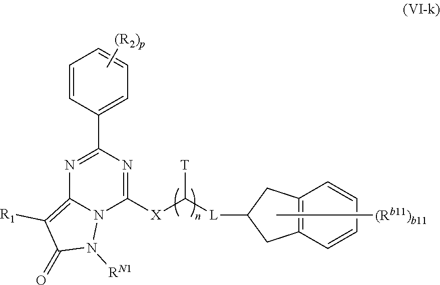

or a pharmaceutically acceptable salt, solvate, hydrate, polymorph, co-crystal, tautomer, stereoisomer, isotopically labeled derivative, or prodrug thereof, wherein R^{N1}, R^{b11}, R₁, R₂, T, X, L, n, p, and b11 are as described herein.

In certain embodiments, a provided compound is of Formula (VI-m):

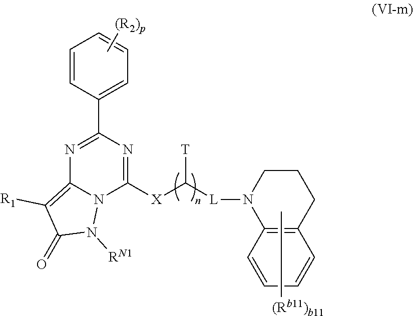

or a pharmaceutically acceptable salt, solvate, hydrate, polymorph, co-crystal, tautomer, stereoisomer, isotopically labeled derivative, or prodrug thereof, wherein R^{N1}, R^{b11}, R₁, R₂, T, X, L, n, p, and b11 are as described herein.

In certain embodiments, a provided compound is of Formula (VI-n):

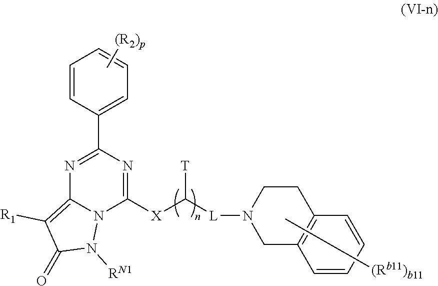

or a pharmaceutically acceptable salt, solvate, hydrate, polymorph, co-crystal, tautomer, stereoisomer, isotopically labeled derivative, or prodrug thereof, wherein R^{N1}, R^{b11}, R₁, R₂, T, X, L, n, p, and b11 are as described herein.

In certain embodiments, a provided compound is of Formula (VII):

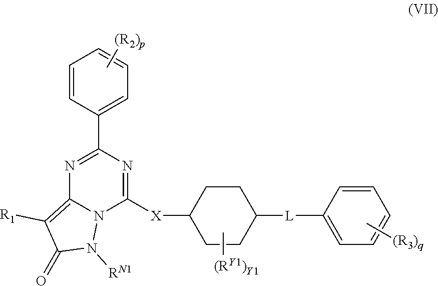

or a pharmaceutically acceptable salt, solvate, hydrate, polymorph, co-crystal, tautomer, stereoisomer, isotopically labeled derivative, or prodrug thereof, wherein R^{N1}, R^{Y1}, X, L, R₁, R₂, R₃, p, Y1, and q are as described herein.

In certain embodiments, a provided compound is of Formula (VII-i):

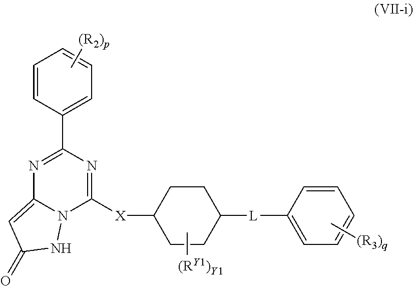

or a pharmaceutically acceptable salt, solvate, hydrate, polymorph, co-crystal, tautomer, stereoisomer, isotopically labeled derivative, or prodrug thereof, wherein R^{Y1}, X, L, R₂, R₃, p, Y1, and q are as described herein.

As generally defined herein, R^{Y1} is independently selected from the group consisting of hydrogen, halogen, or optionally substituted alkyl.

As generally defined herein, Y1 is independently 0, 1, 2, 3, 4, 5, 6, 7, or 8. In certain embodiments, Y1 is 0. In certain embodiments, Y1 is 1. In certain embodiments, Y1 is 2. In certain embodiments, Y1 is 3. In certain embodiments, Y1 is 4. In certain embodiments, Y1 is 5. In certain embodiments, Y1 is 6. In certain embodiments, Y1 is 7. In certain embodiments, Y1 is 8.

In certain embodiments, a provided compound is one of the following formulae:

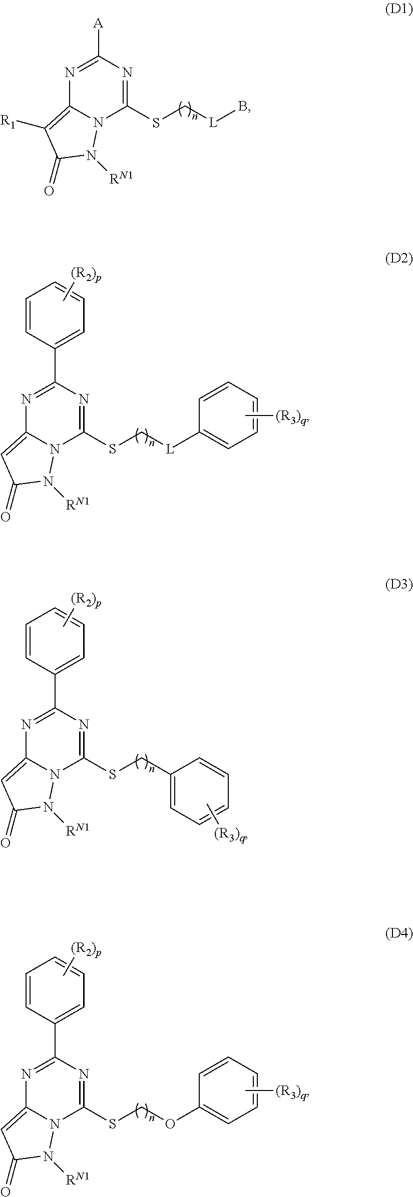

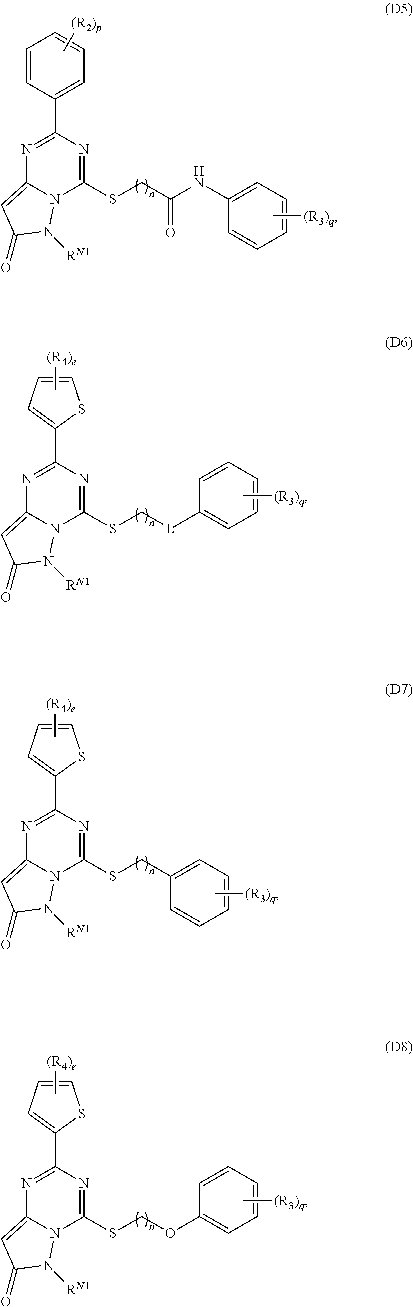

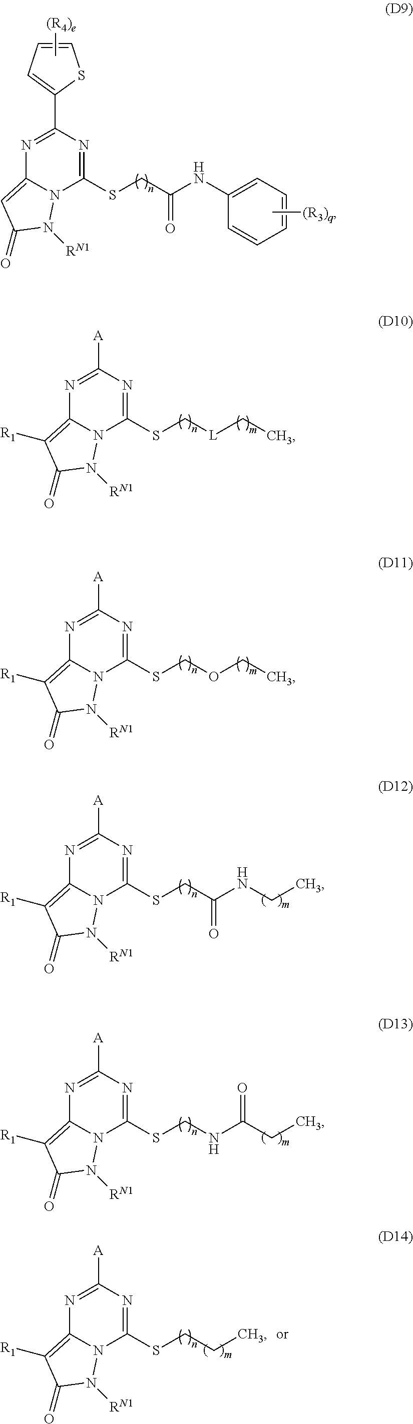

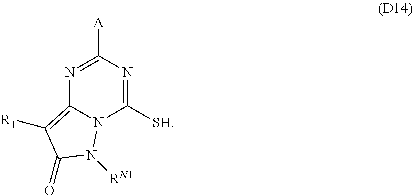

In certain embodiments, a provided compound is of the following formula:

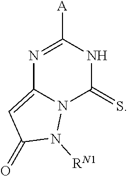

In certain embodiments, a provided compound is one of the following formulae:

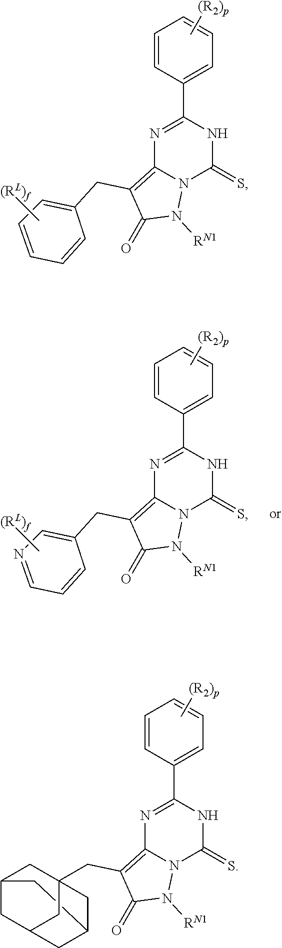

In certain embodiments, a provided compound is one of the following formulae:

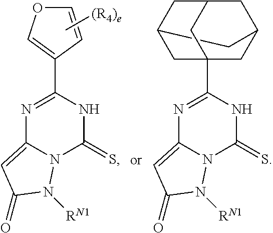

In certain embodiments, a provided compound is one of the following formulae:

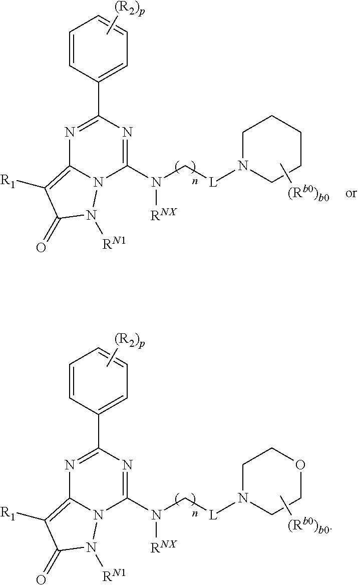

In certain embodiments, a provided compound is one of the following formulae:

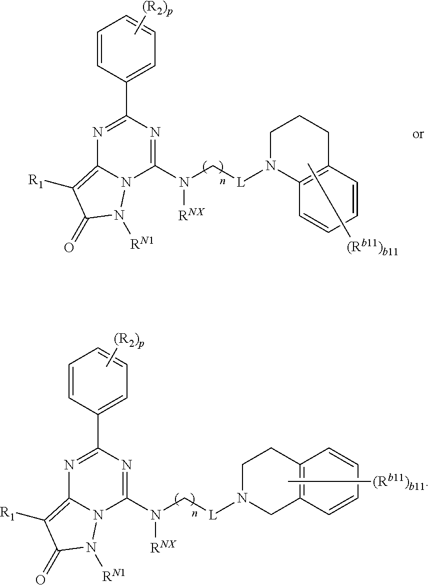

In certain embodiments, a provided compound is one of the following formulae:

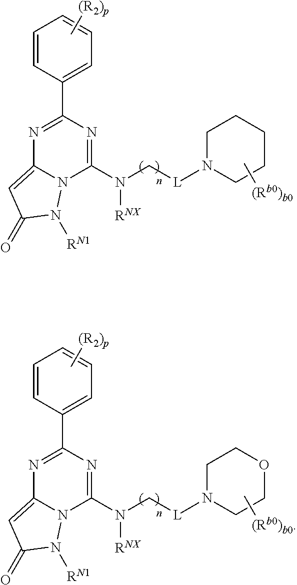

In certain embodiments, a provided compound is one of the following formulae:

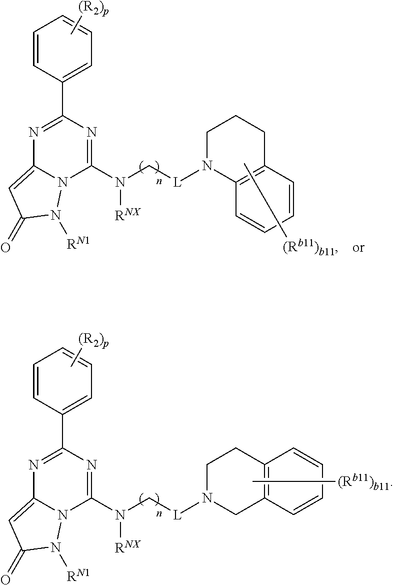

In certain embodiments, a provided compound is one of the following formulae:

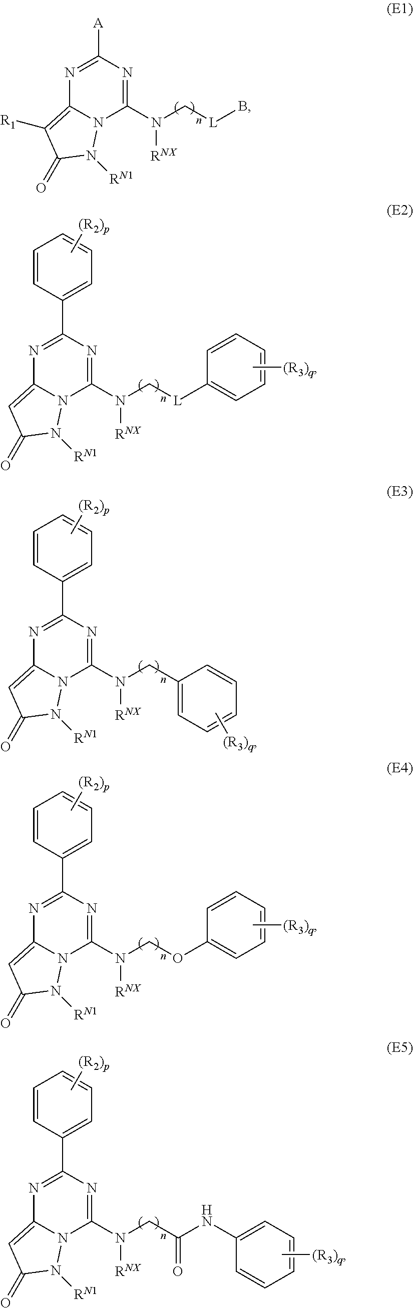

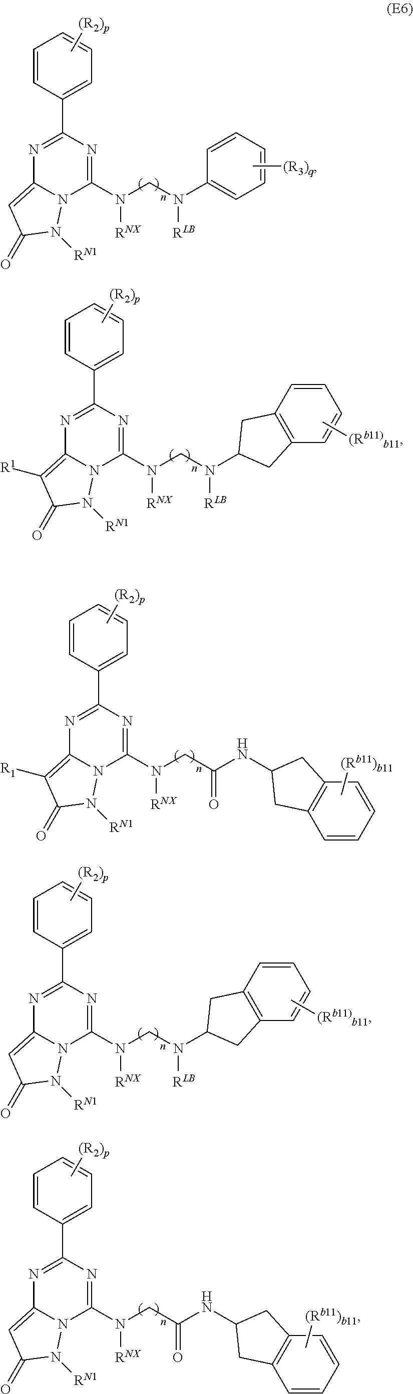

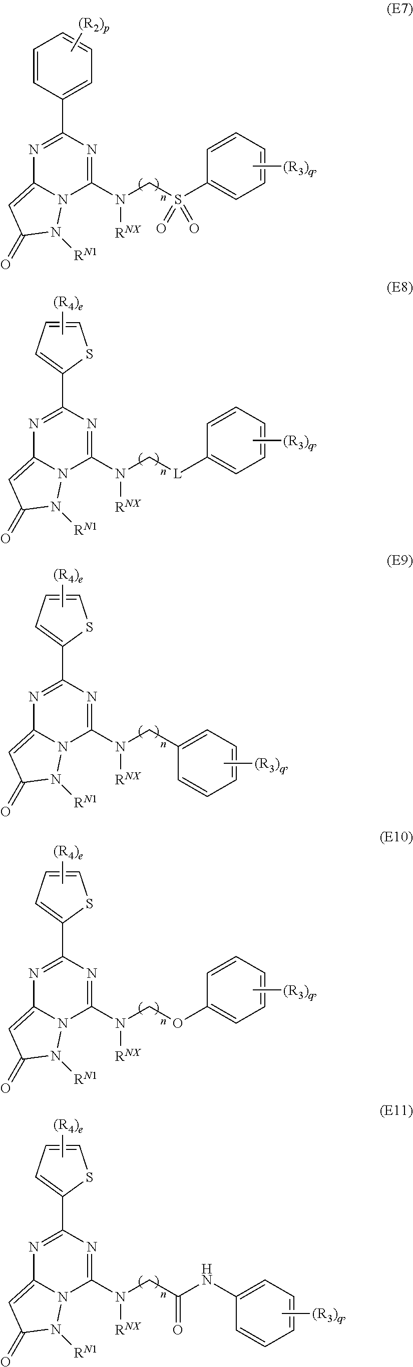

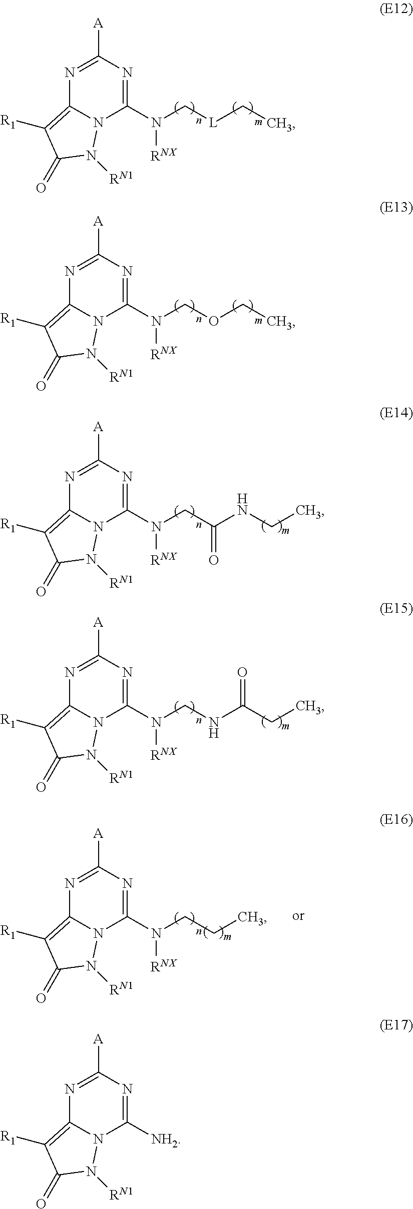

In certain embodiments, a provided compound is one of the following formulae:

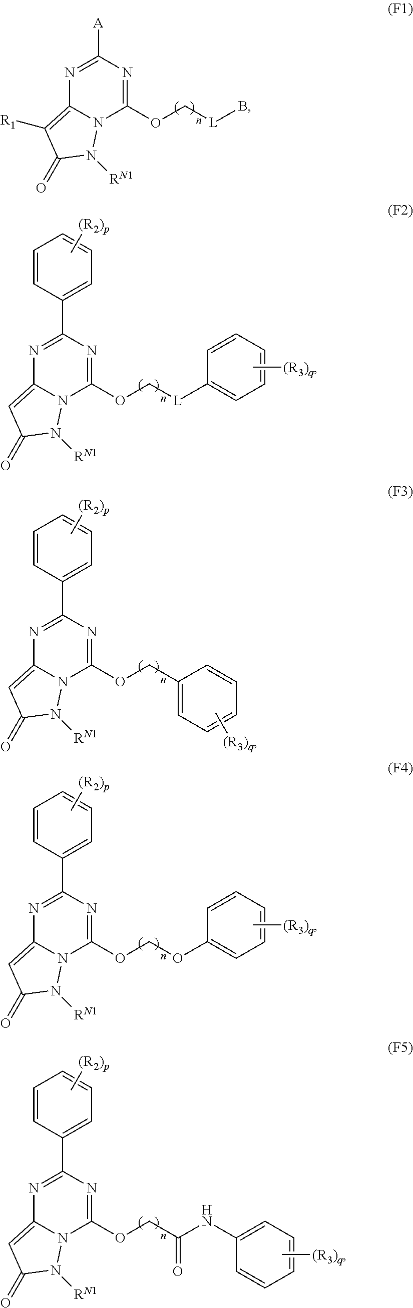

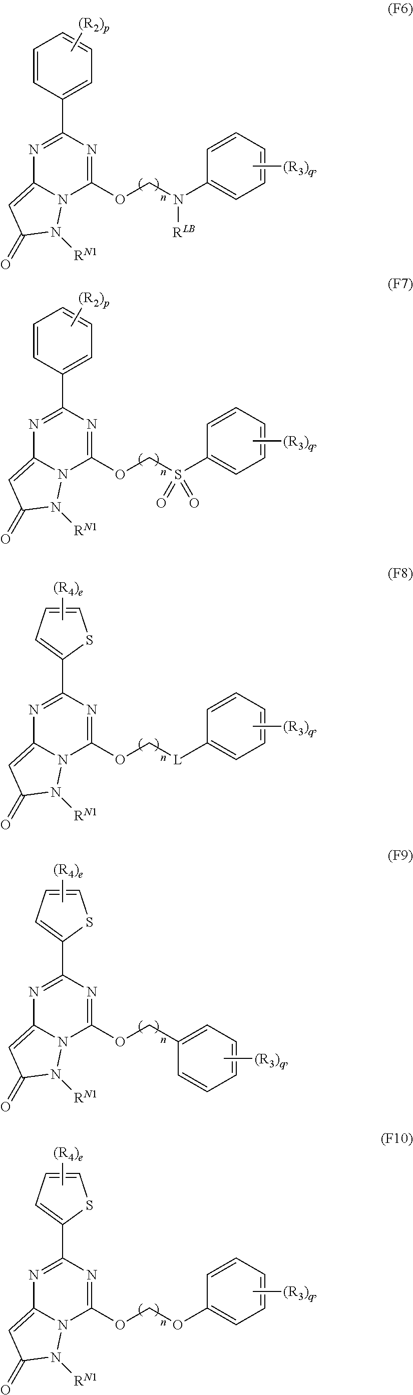

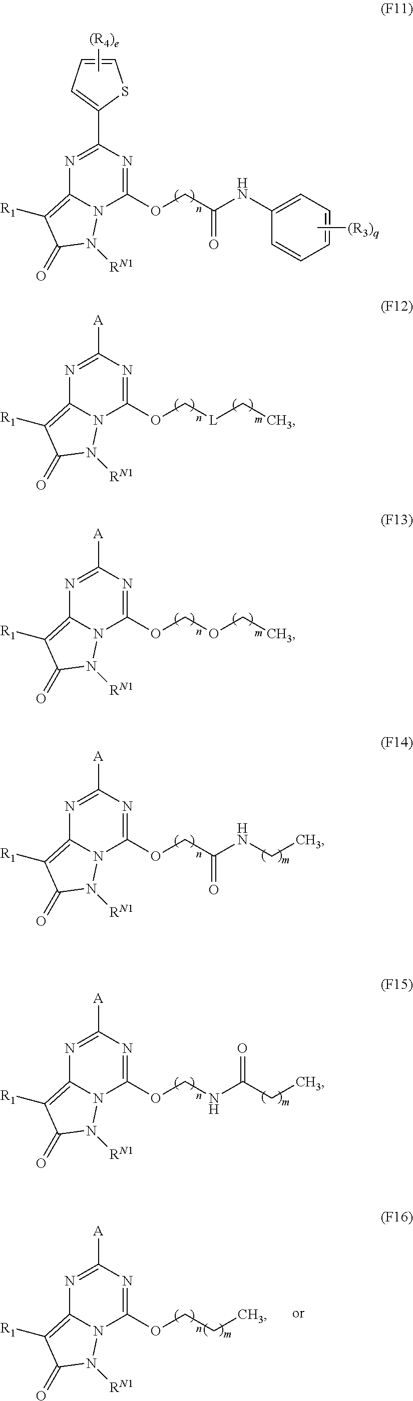

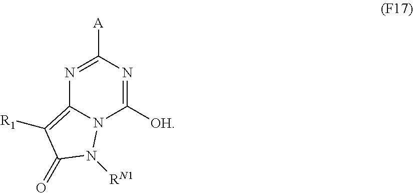

In certain embodiments, exemplary compounds of Formulae (I′) and (IA) include, but are not limited to:

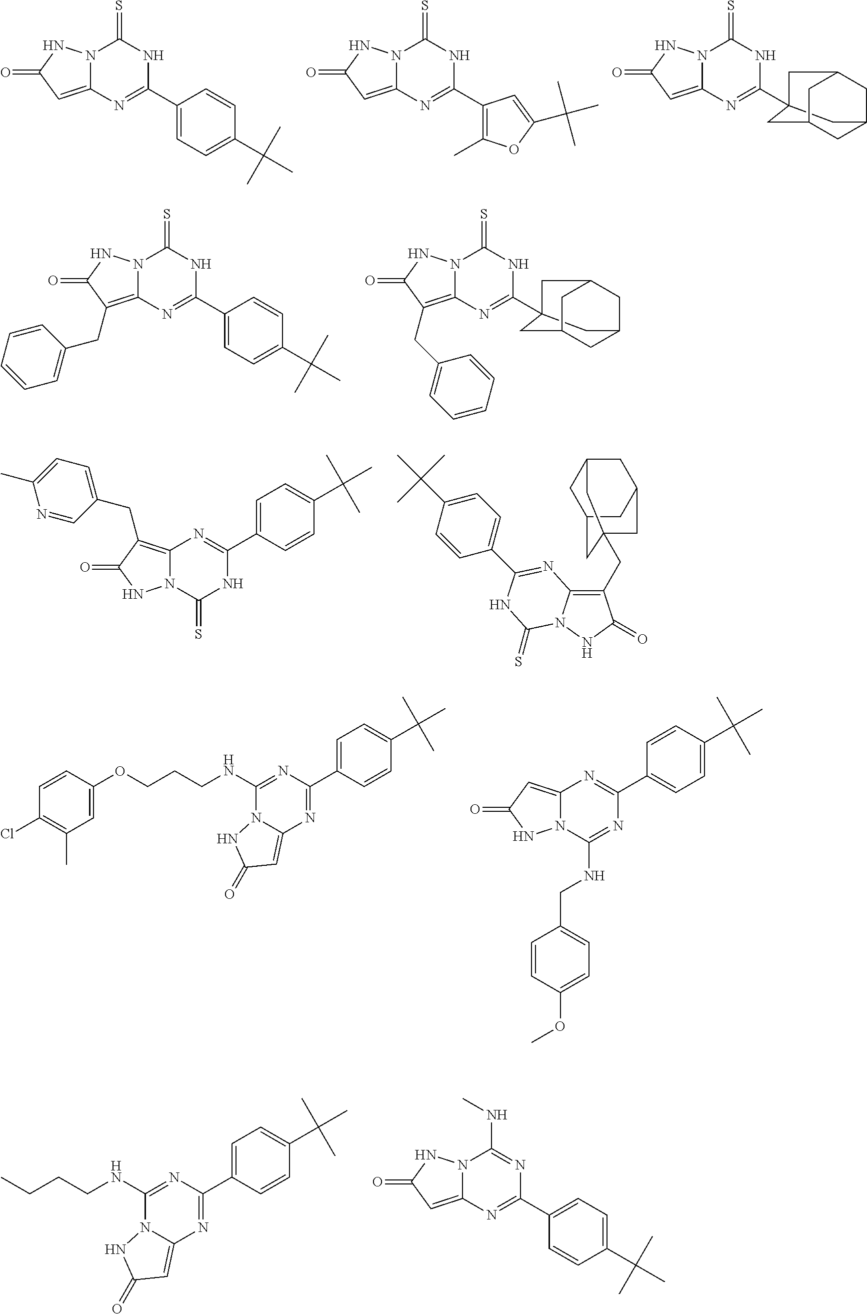

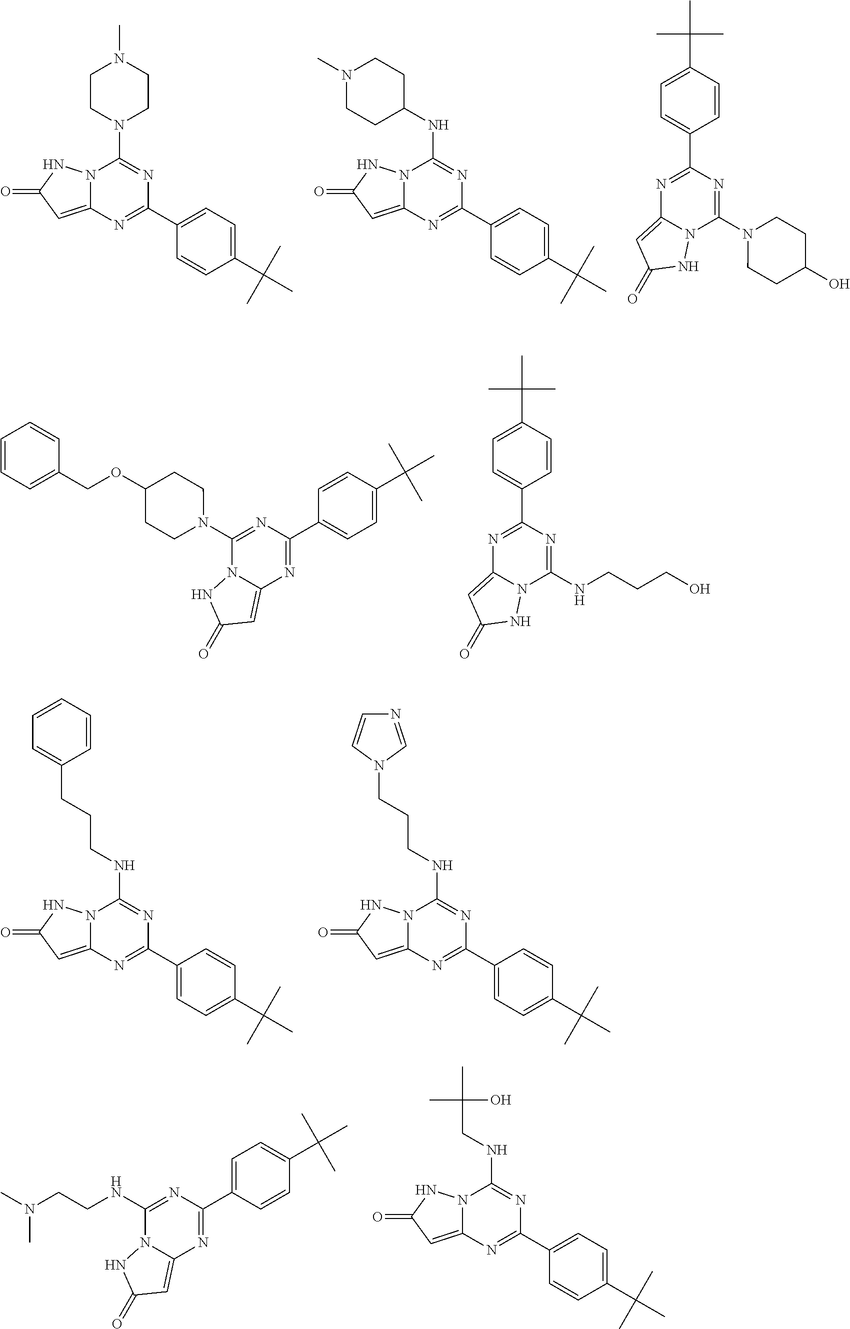

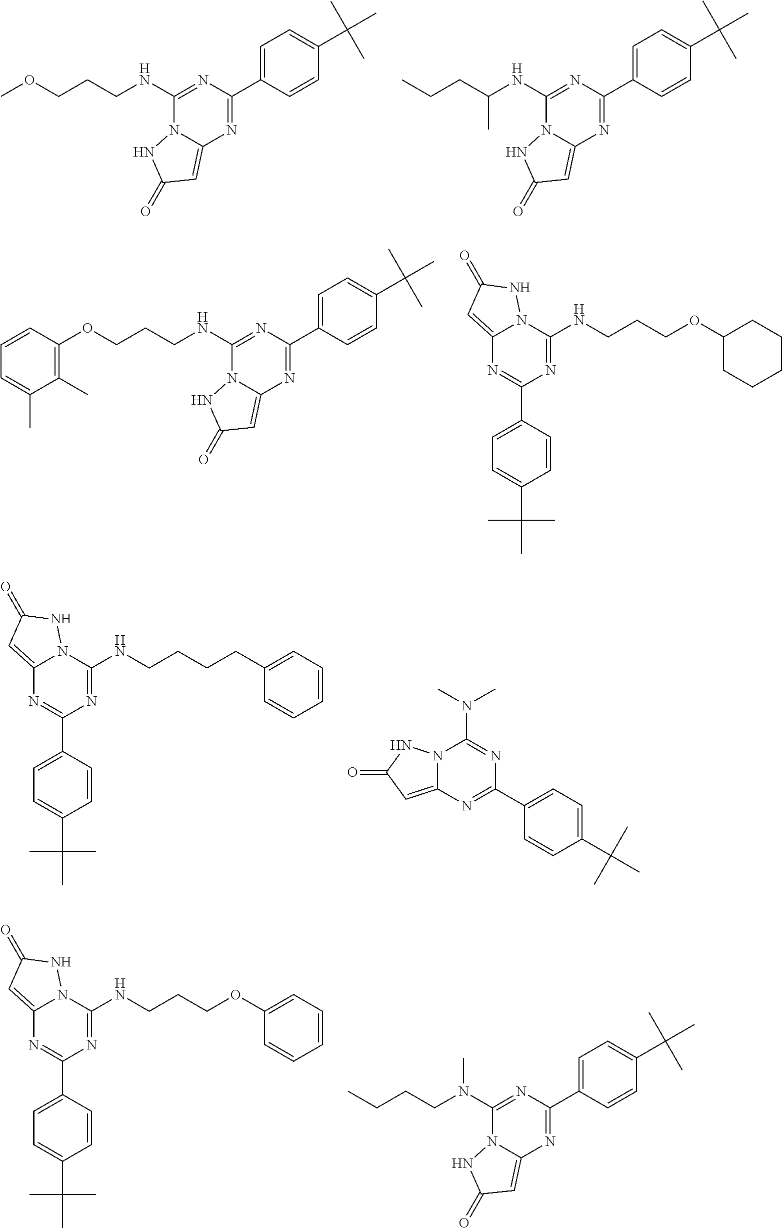

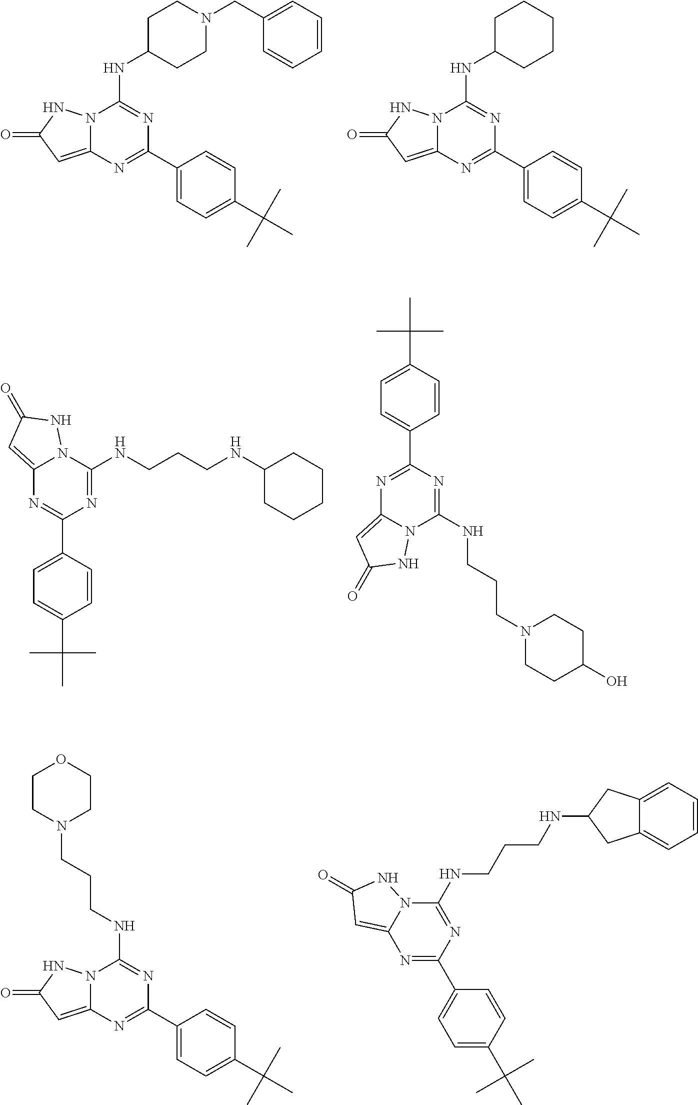 [SMILES: CC(C)(C)c1ccc(-c2nc(NC3CCN(Cc4ccccc4)CC3)n3[nH]c(=O)cc3n2)cc1 ; CC(C)(C)c1ccc(-c2nc(NC3CCCCC3)n3[nH]c(=O)cc3n2)cc1 ; CC(C)(C)c1ccc(-c2nc(NCCCNC3CCCCC3)n3[nH]c(=O)cc3n2)cc1 ; CC(C)(C)c1ccc(-c2nc(NCCCN3CCC(O)CC3)n3[nH]c(=O)cc3n2)cc1 ; CC(C)(C)c1ccc(-c2nc(NCCCN3CCOCC3)n3[nH]c(=O)cc3n2)cc1 ; CC(C)(C)c1ccc(-c2nc(NCCCNC3Cc4ccccc4C3)n3[nH]c(=O)cc3n2)cc1]

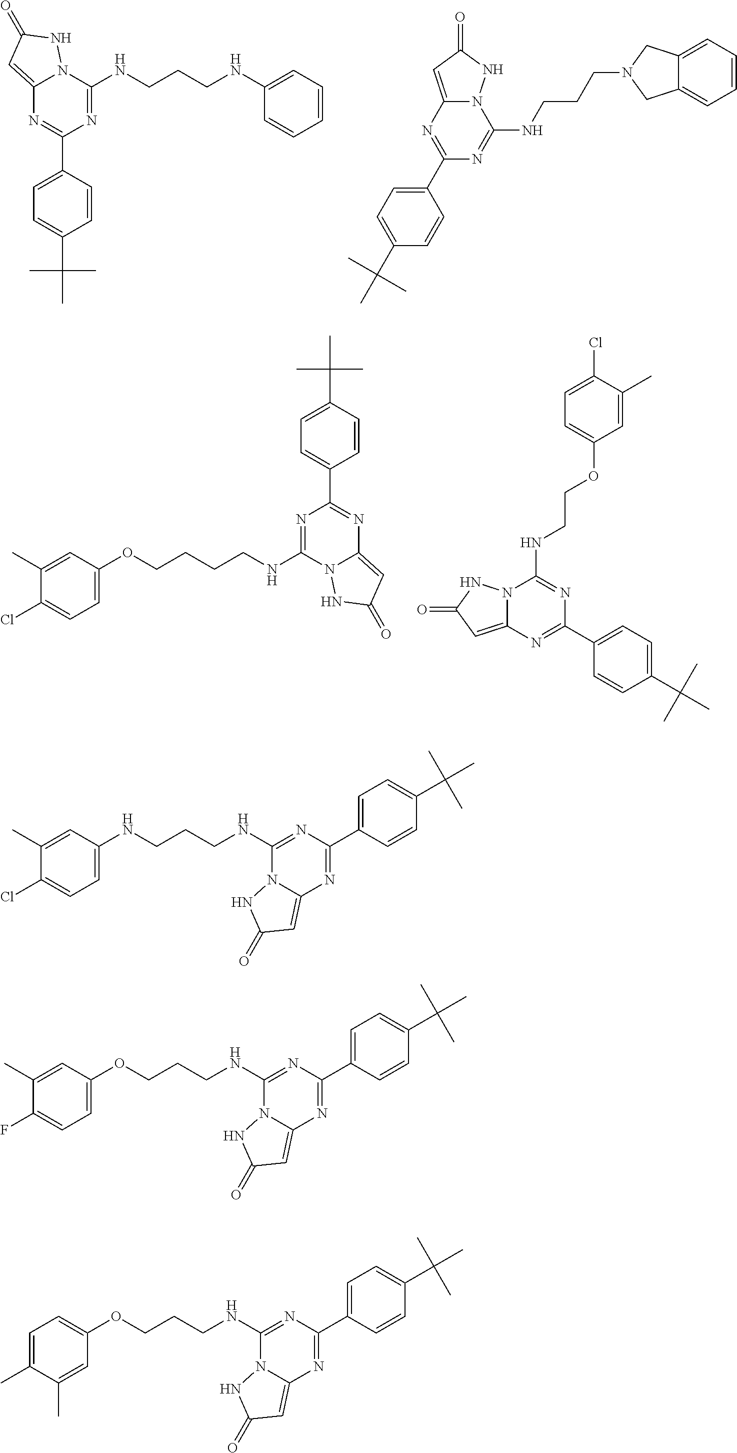

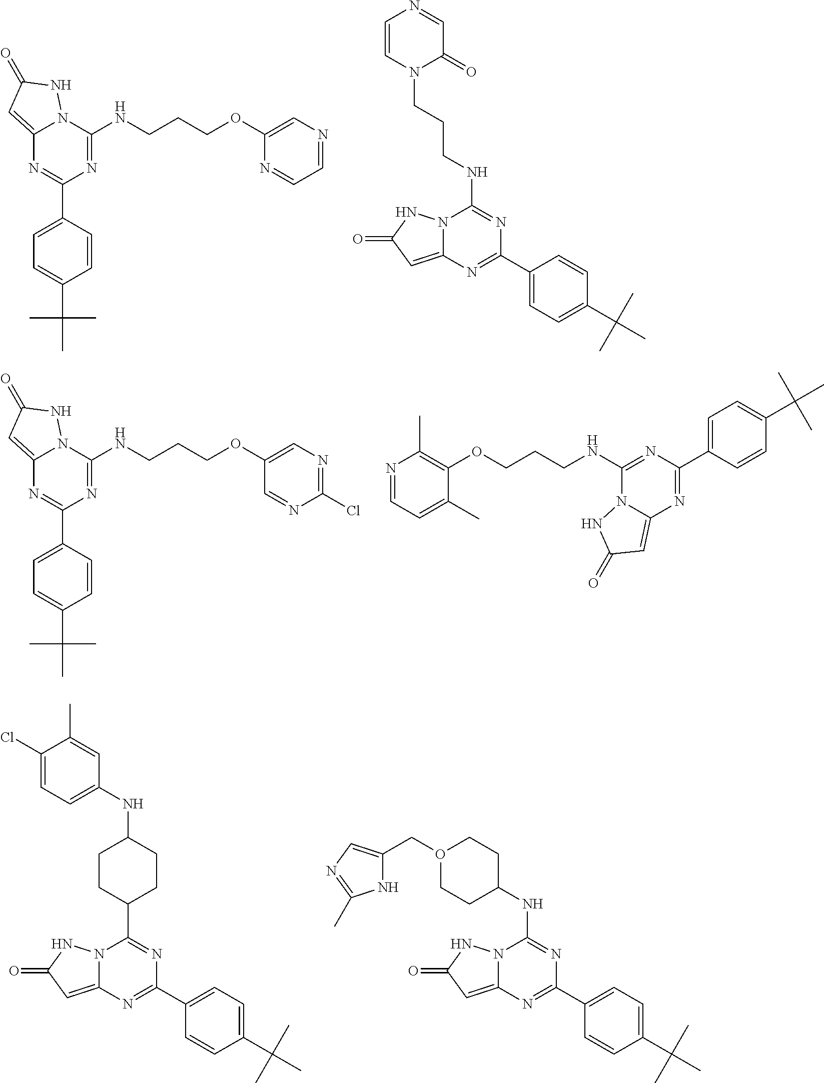

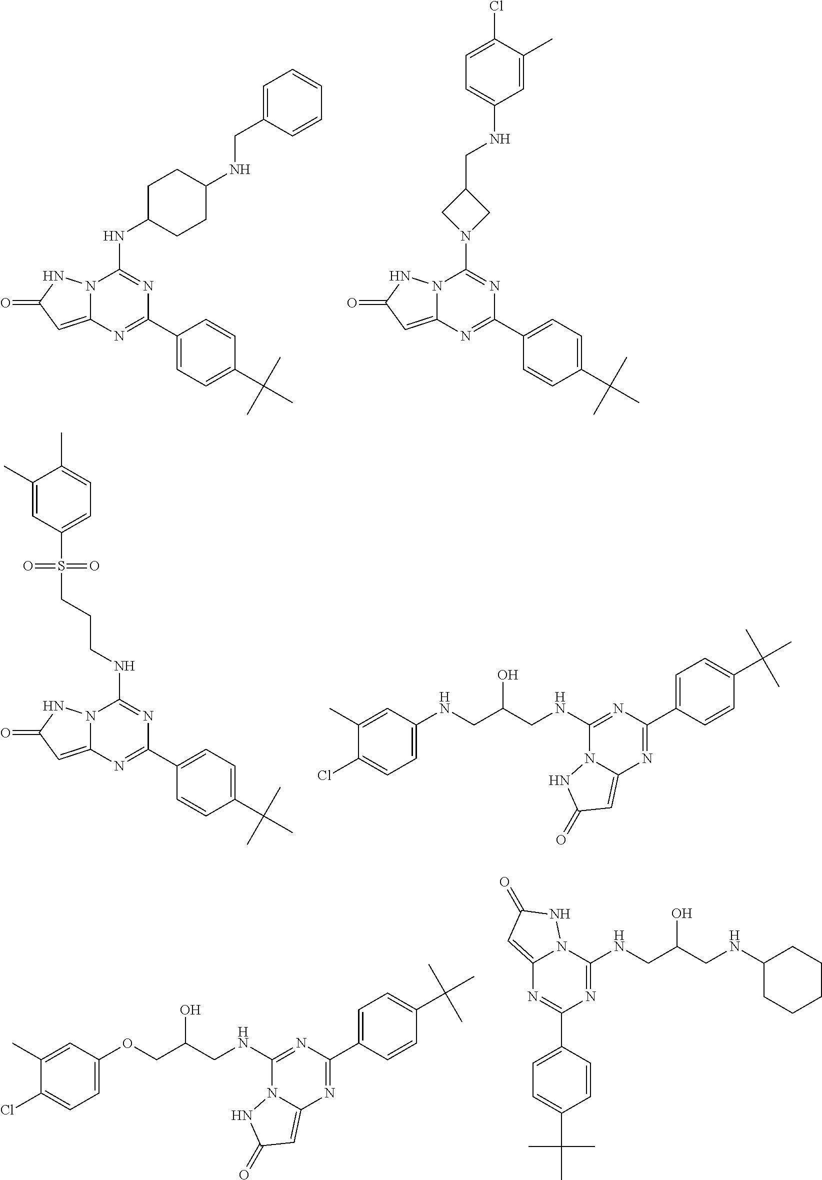 [SMILES: CC(C)(C)c1ccc(-c2nc(NC3CCC(NCc4ccccc4)CC3)n3[nH]c(=O)cc3n2)cc1 ; Cc1cc(NCC2CN(c3nc(-c4ccc(C(C)(C)C)cc4)nc4cc(=O)[nH]n34)C2)ccc1Cl ; Cc1ccc(S(=O)(=O)CCCNc2nc(-c3ccc(C(C)(C)C)cc3)nc3cc(=O)[nH]n23)cc1C ; Cc1cc(NCC(O)CNc2nc(-c3ccc(C(C)(C)C)cc3)nc3cc(=O)[nH]n23)ccc1Cl ; Cc1cc(OCC(O)CNc2nc(-c3ccc(C(C)(C)C)cc3)nc3cc(=O)[nH]n23)ccc1Cl ; CC(C)(C)c1ccc(-c2nc(NCC(O)CNC3CCCCC3)n3[nH]c(=O)cc3n2)cc1]

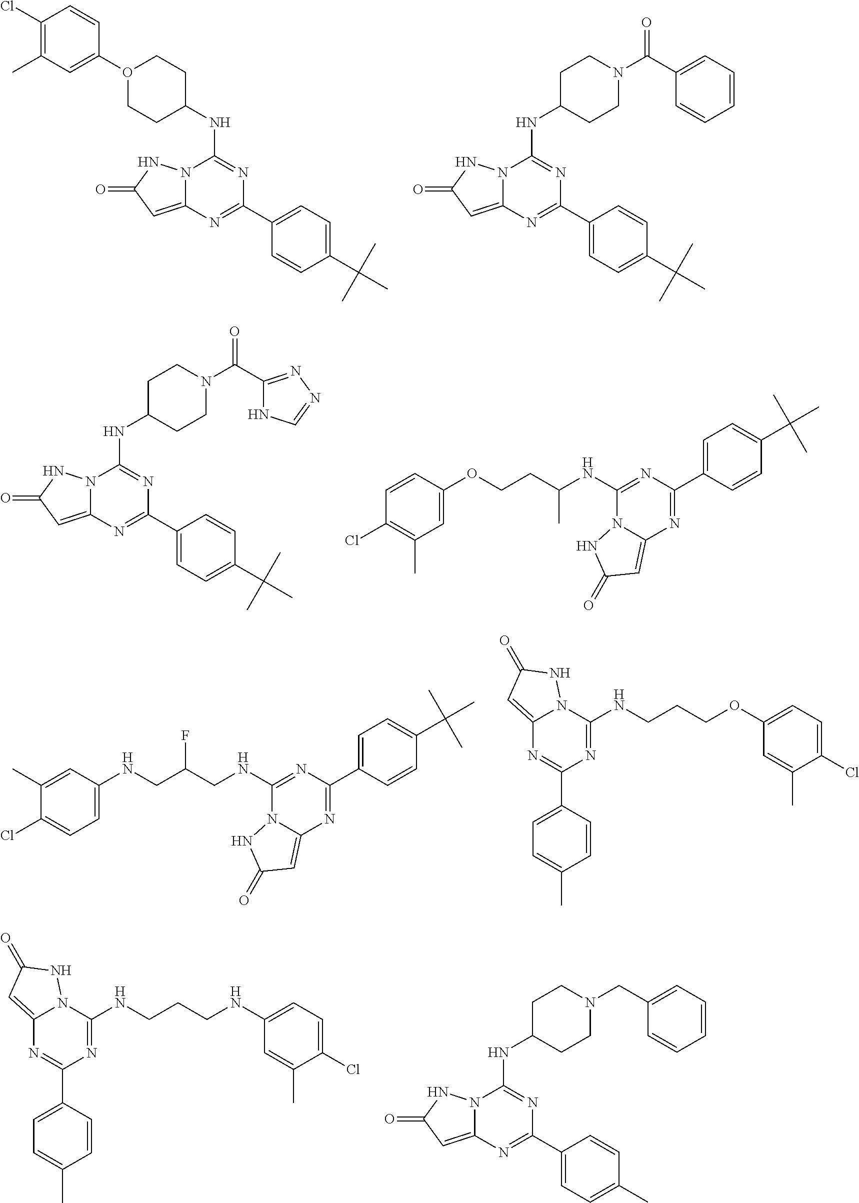

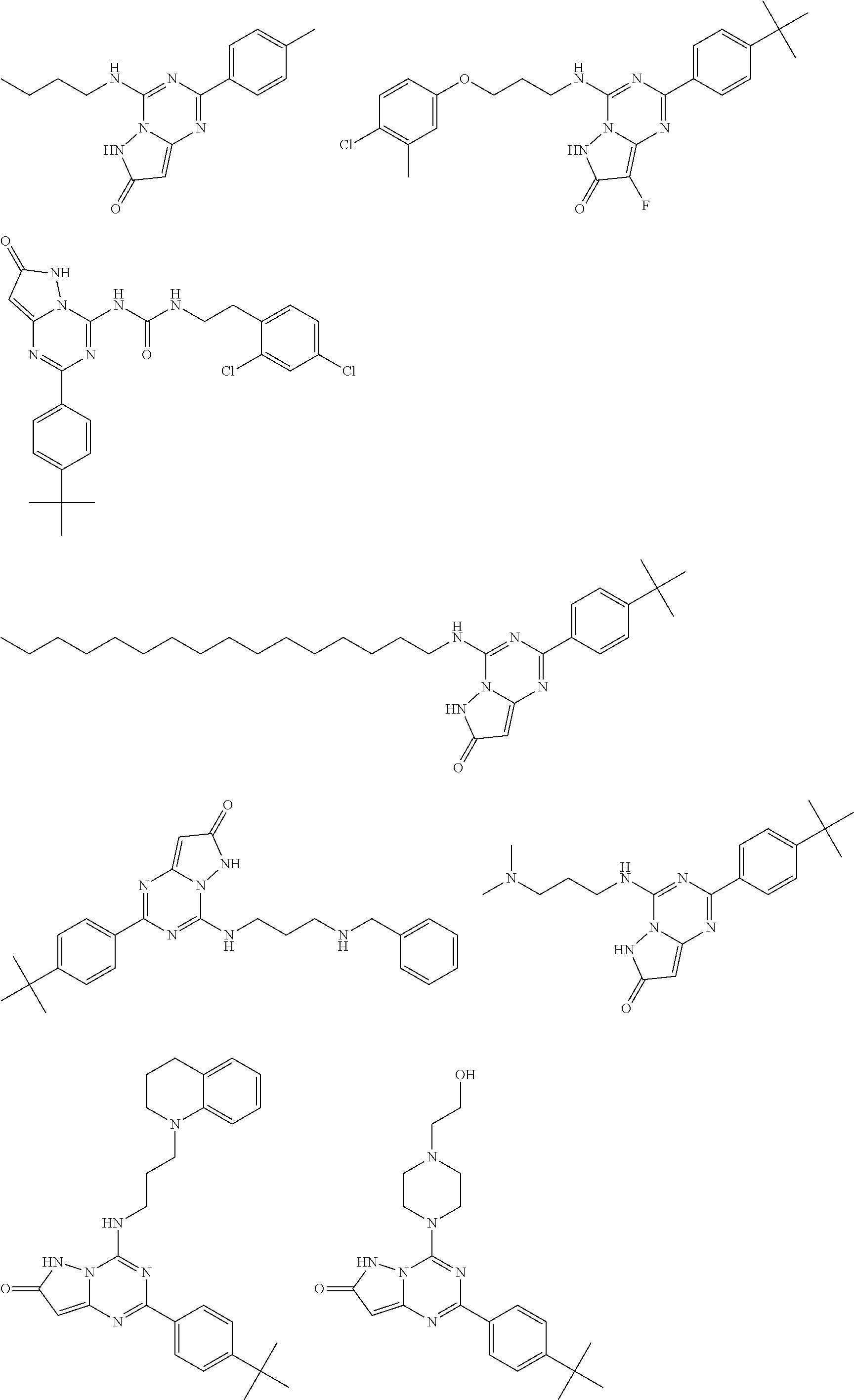

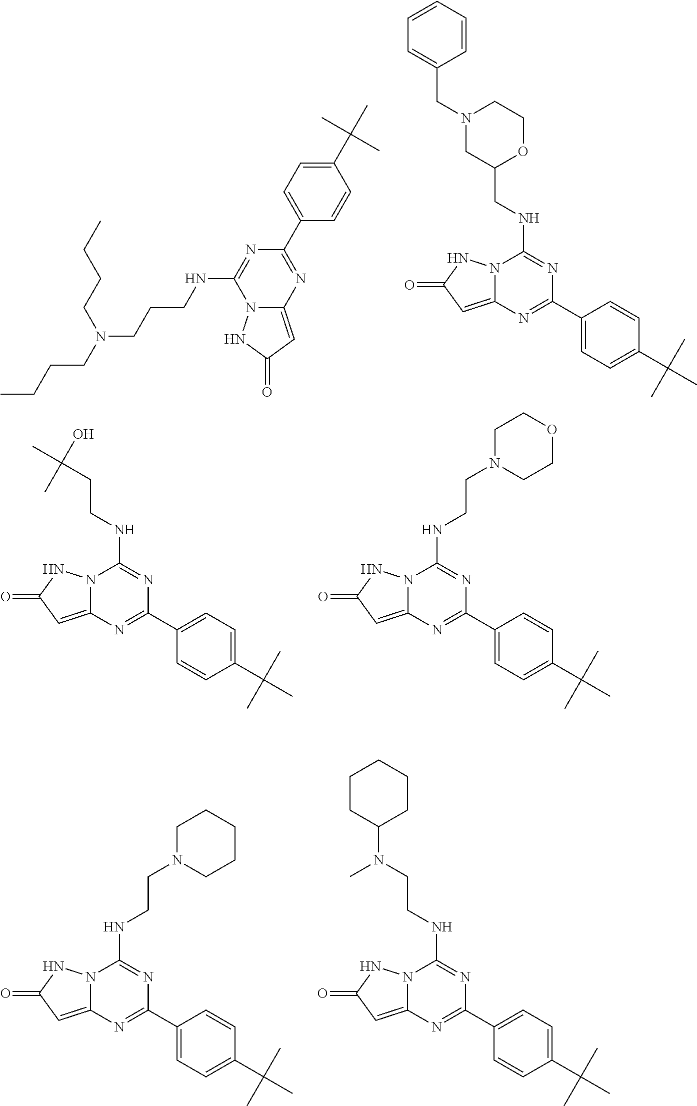 [SMILES: CCCCN(CCCC)CCCNc1nc(-c2ccc(C(C)(C)C)cc2)nc2cc(=O)[nH]n12 ; CC(C)(C)c1ccc(-c2nc(NCC3CN(Cc4ccccc4)CCO3)n3[nH]c(=O)cc3n2)cc1 ; CC(C)(O)CCNc1nc(-c2ccc(C(C)(C)C)cc2)nc2cc(=O)[nH]n12 ; CC(C)(C)c1ccc(-c2nc(NCCN3CCOCC3)n3[nH]c(=O)cc3n2)cc1 ; CC(C)(C)c1ccc(-c2nc(NCCN3CCCCC3)n3[nH]c(=O)cc3n2)cc1 ; CN(CCNc1nc(-c2ccc(C(C)(C)C)cc2)nc2cc(=O)[nH]n12)C1CCCCC1]

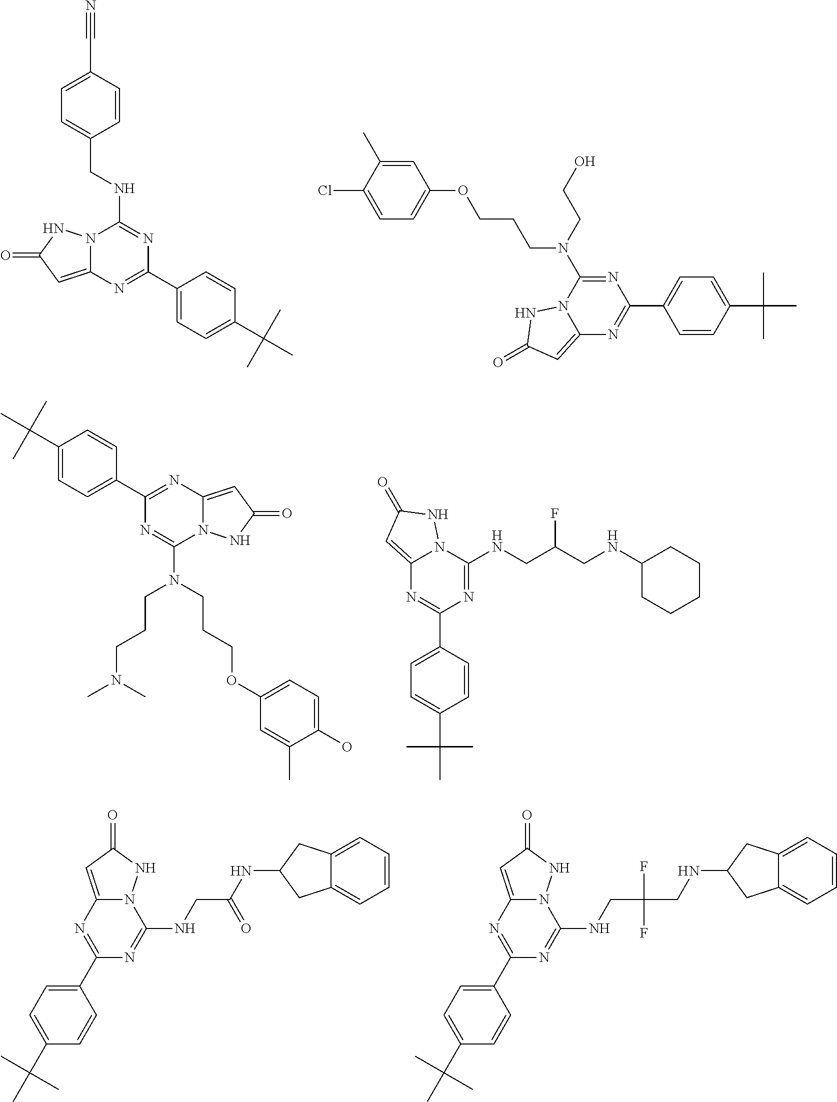 [SMILES: CC(C)(C)c1ccc(-c2nc(NCc3ccc(C#N)cc3)n3[nH]c(=O)cc3n2)cc1 ; Cc1cc(OCCCN(CCO)c2nc(-c3ccc(C(C)(C)C)cc3)nc3cc(=O)[nH]n23)ccc1Cl ; Cc1cc(OCCCN(CCCN(C)C)c2nc(-c3ccc(C(C)(C)C)cc3)nc3cc(=O)[nH]n23)ccc1O ; CC(C)(C)c1ccc(-c2nc(NCC(F)CNC3CCCCC3)n3[nH]c(=O)cc3n2)cc1 ; CC(C)(C)c1ccc(-c2nc(NCC(=O)NC3Cc4ccccc4C3)n3[nH]c(=O)cc3n2)cc1 ; CC(C)(C)c1ccc(-c2nc(NCC(F)(F)CNC3Cc4ccccc4C3)n3[nH]c(=O)cc3n2)cc1]

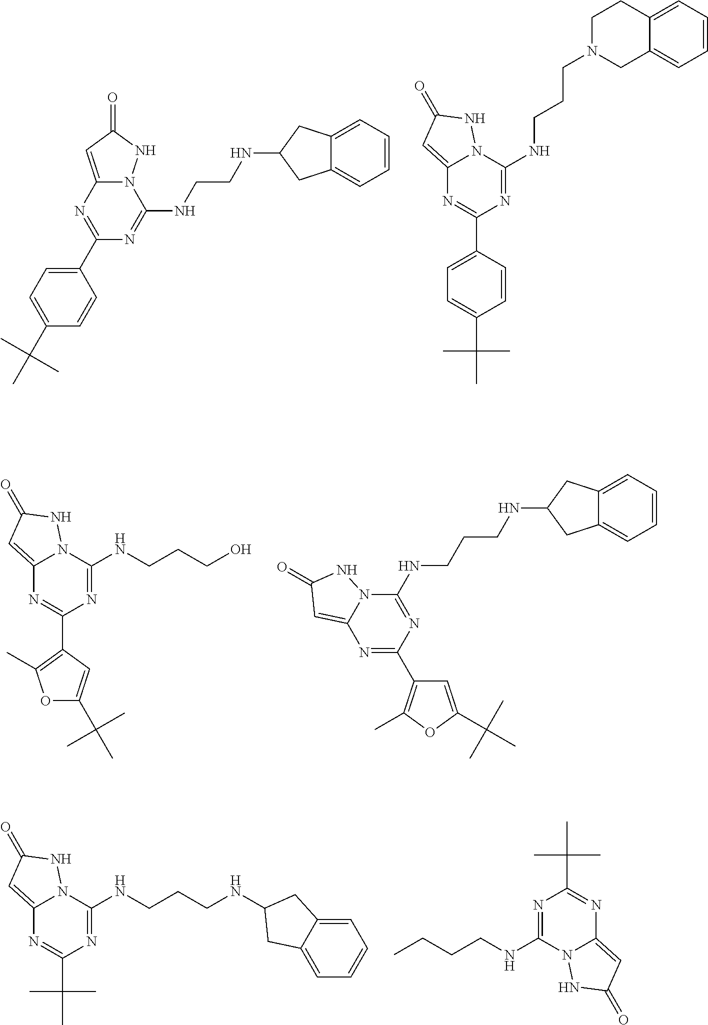

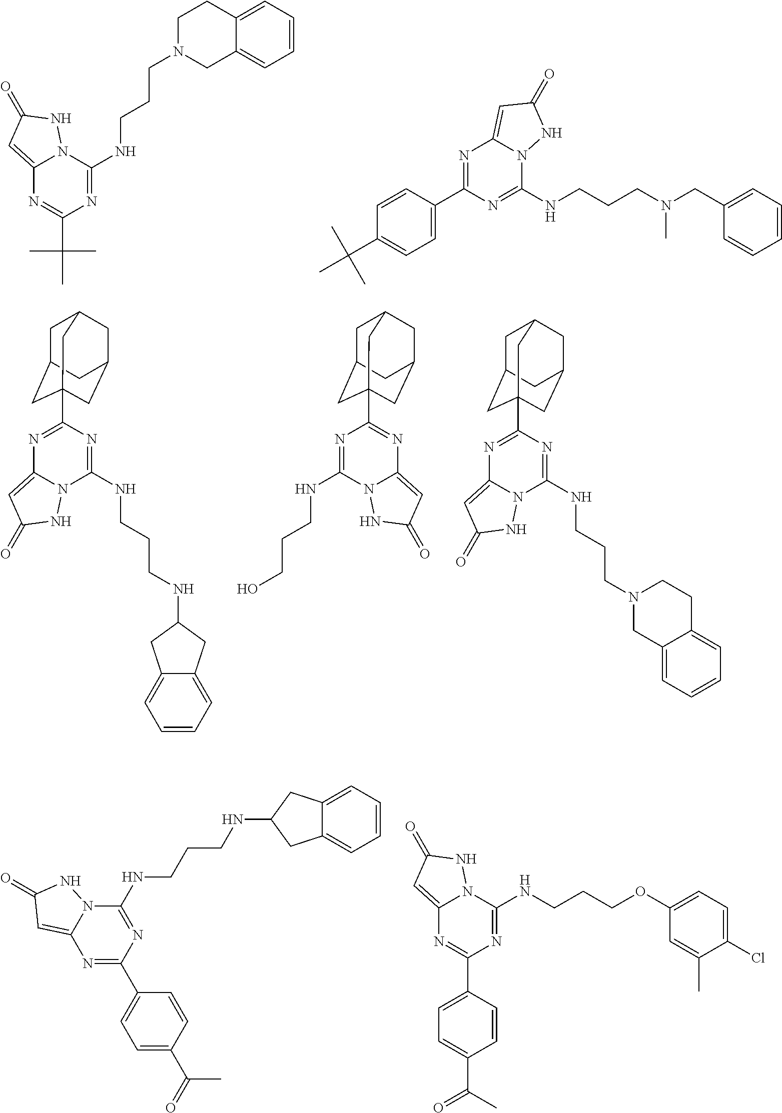

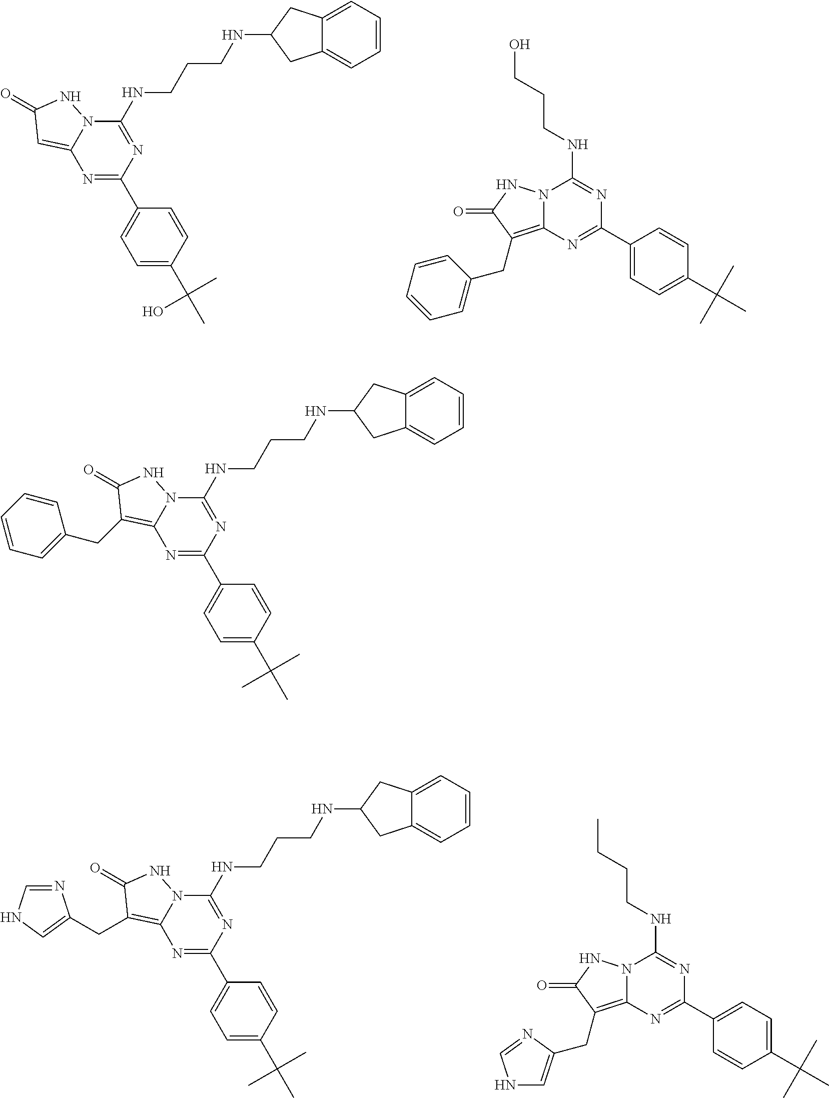 [SMILES: CC(C)(O)c1ccc(-c2nc(NCCCNC3Cc4ccccc4C3)n3[nH]c(=O)cc3n2)cc1 ; CC(C)(C)c1ccc(-c2nc(NCCCO)n3[nH]c(=O)c(Cc4ccccc4)c3n2)cc1 ; CC(C)(C)c1ccc(-c2nc(NCCCNC3Cc4ccccc4C3)n3[nH]c(=O)c(Cc4ccccc4)c3n2)cc1 ; CC(C)(C)c1ccc(-c2nc(NCCCNC3Cc4ccccc4C3)n3[nH]c(=O)c(Cc4c[nH]cn4)c3n2)cc1 ; CCCCNc1nc(-c2ccc(C(C)(C)C)cc2)nc2c(Cc3c[nH]cn3)c(=O)[nH]n12]

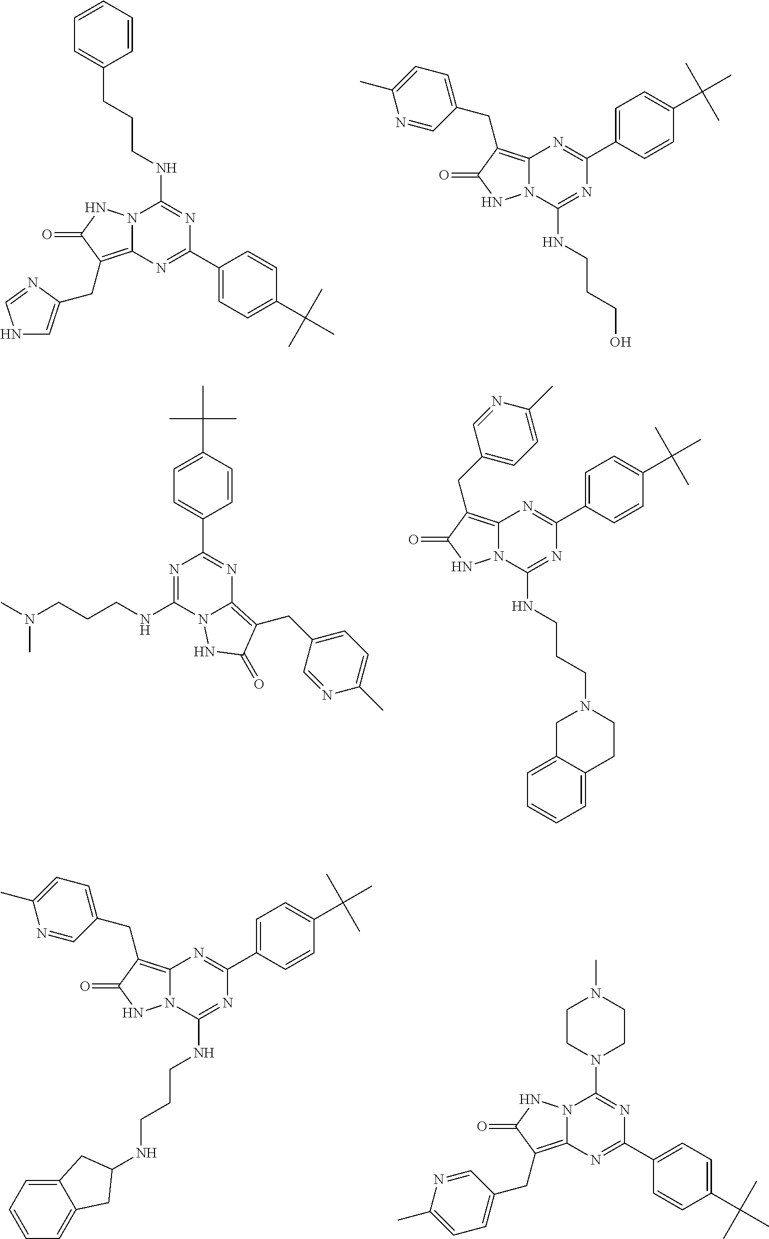

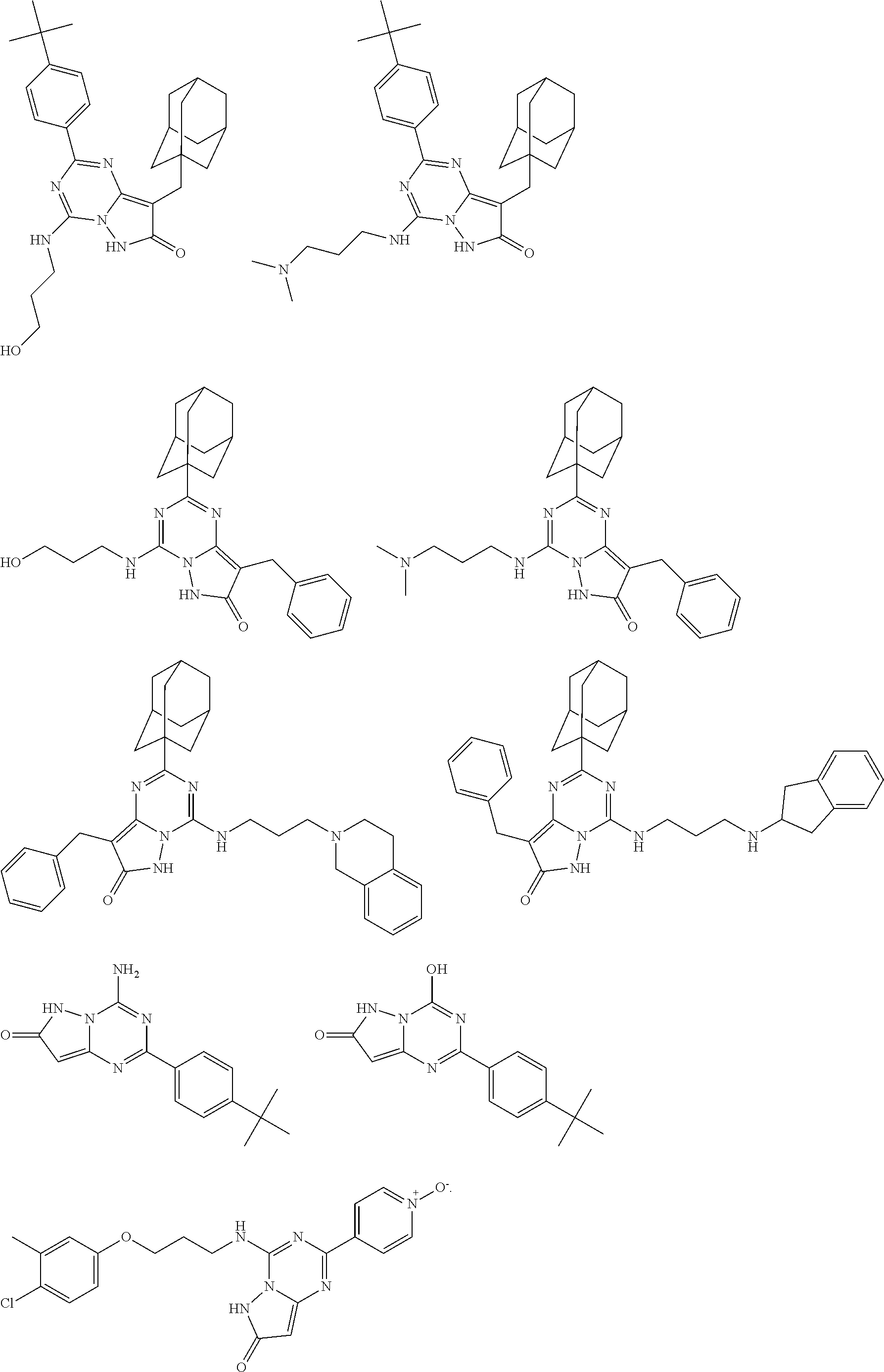

In certain embodiments, exemplary compounds of Formula (I) include, but are not limited to:

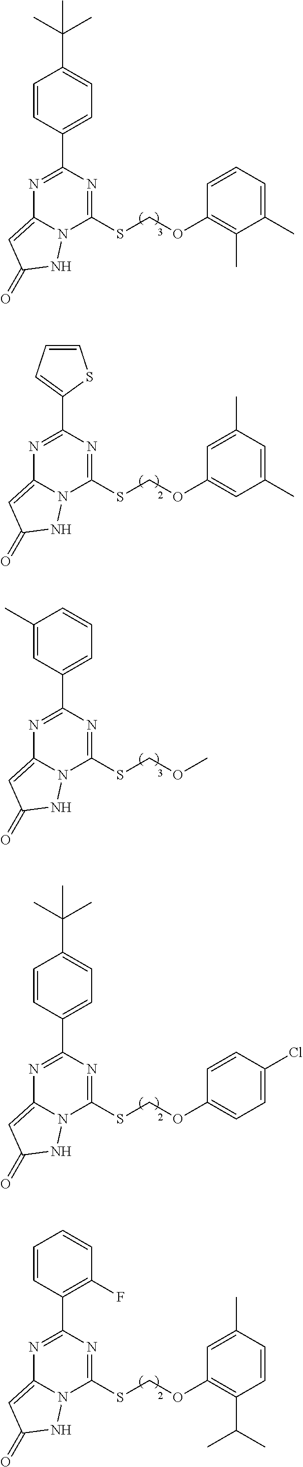

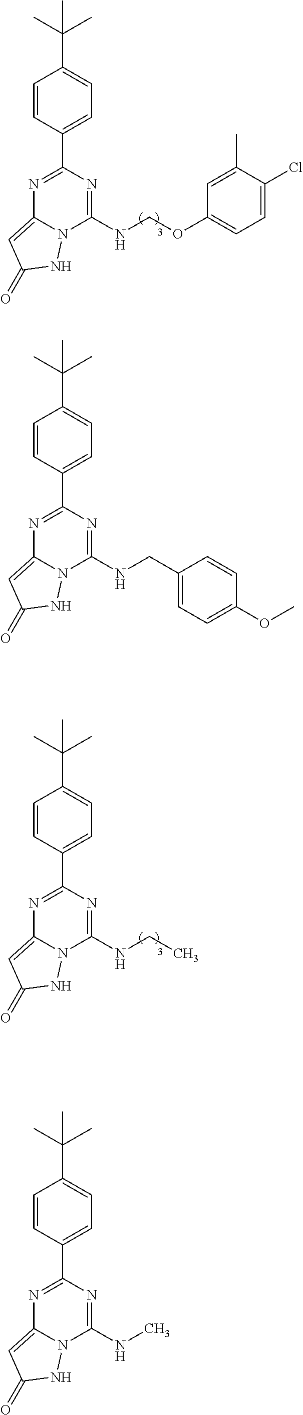

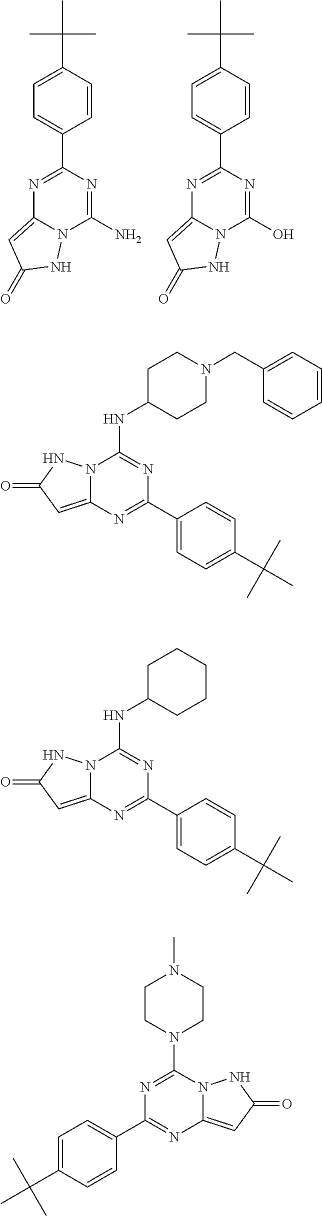

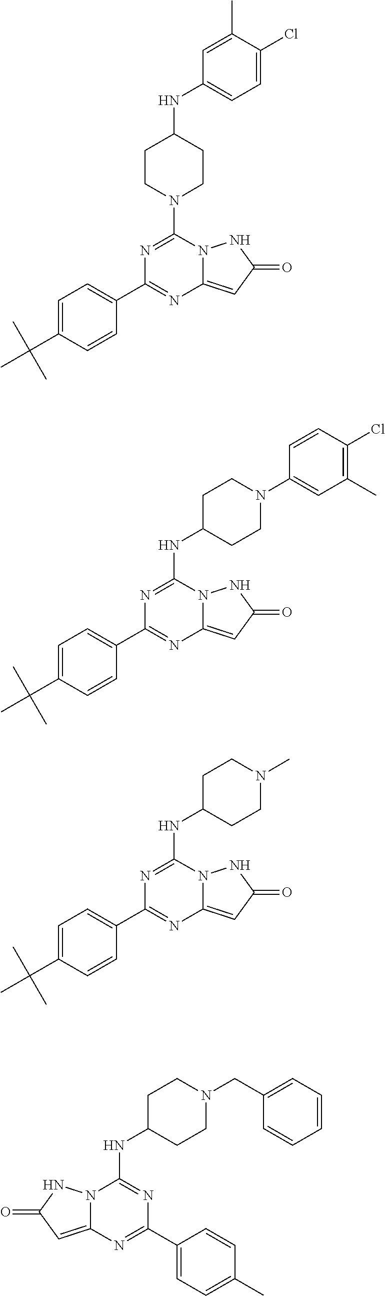

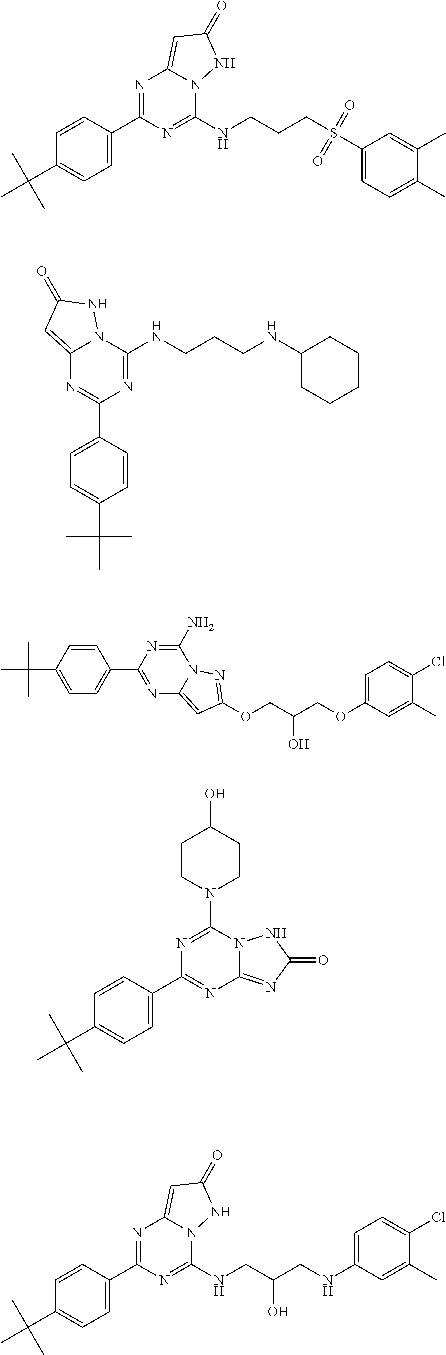

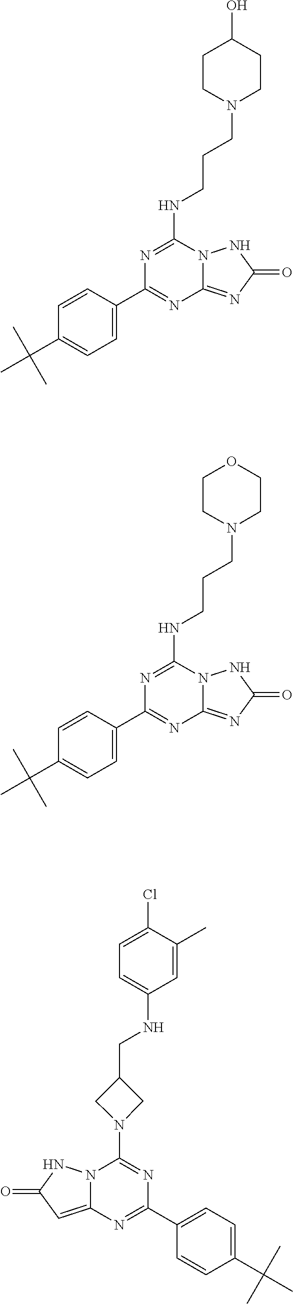 [SMILES: CC(C)(C)c1ccc(-c2nc(NCCCN3CCC(O)CC3)n3[nH]c(=O)nc3n2)cc1 ; CC(C)(C)c1ccc(-c2nc(NCCCN3CCOCC3)n3[nH]c(=O)nc3n2)cc1 ; Cc1cc(NCC2CN(c3nc(-c4ccc(C(C)(C)C)cc4)nc4cc(=O)[nH]n34)C2)ccc1Cl]

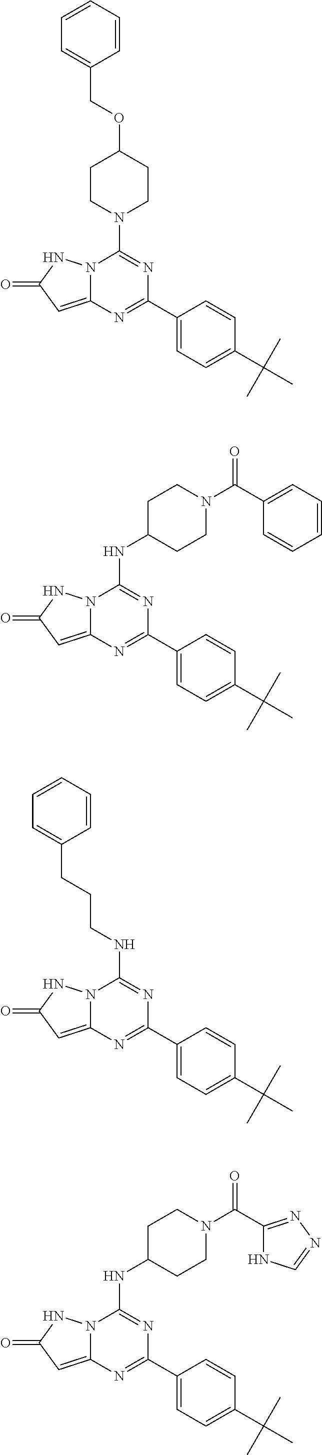 [SMILES: CC(C)(C)c1ccc(-c2nc(N3CCC(OCc4ccccc4)CC3)n3[nH]c(=O)cc3n2)cc1 ; CC(C)(C)c1ccc(-c2nc(NC3CCN(C(=O)c4ccccc4)CC3)n3[nH]c(=O)cc3n2)cc1 ; CC(C)(C)c1ccc(-c2nc(NCCCc3ccccc3)n3[nH]c(=O)cc3n2)cc1 ; CC(C)(C)c1ccc(-c2nc(NC3CCN(C(=O)c4nnc[nH]4)CC3)n3[nH]c(=O)cc3n2)cc1]

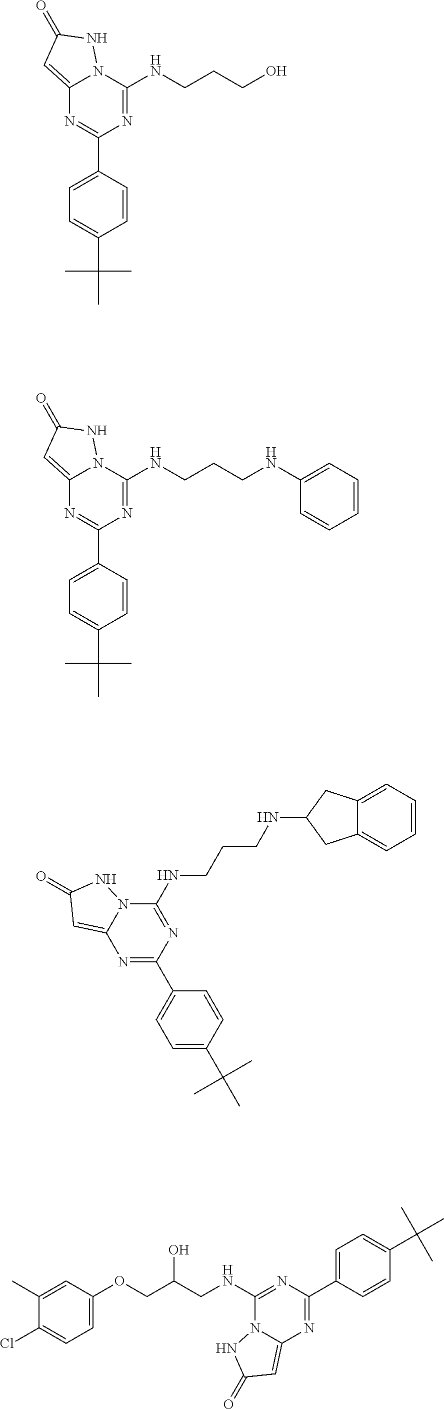 [SMILES: CC(C)(C)c1ccc(-c2nc(NCCCO)n3[nH]c(=O)cc3n2)cc1 ; CC(C)(C)c1ccc(-c2nc(NCCCNc3ccccc3)n3[nH]c(=O)cc3n2)cc1 ; CC(C)(C)c1ccc(-c2nc(NCCCNC3Cc4ccccc4C3)n3[nH]c(=O)cc3n2)cc1 ; Cc1cc(OCC(O)CNc2nc(-c3ccc(C(C)(C)C)cc3)nc3cc(=O)[nH]n23)ccc1Cl]

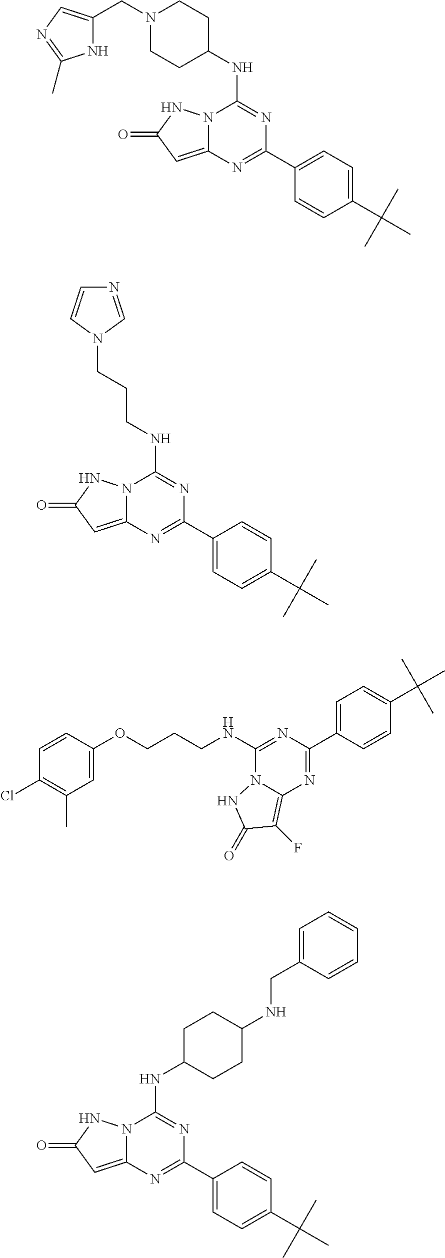 [SMILES: Cc1ncc(CN2CCC(Nc3nc(-c4ccc(C(C)(C)C)cc4)nc4cc(=O)[nH]n34)CC2)[nH]1 ; CC(C)(C)c1ccc(-c2nc(NCCCn3ccnc3)n3[nH]c(=O)cc3n2)cc1 ; Cc1cc(OCCCNc2nc(-c3ccc(C(C)(C)C)cc3)nc3c(F)c(=O)[nH]n23)ccc1Cl ; CC(C)(C)c1ccc(-c2nc(NC3CCC(NCc4ccccc4)CC3)n3[nH]c(=O)cc3n2)cc1]

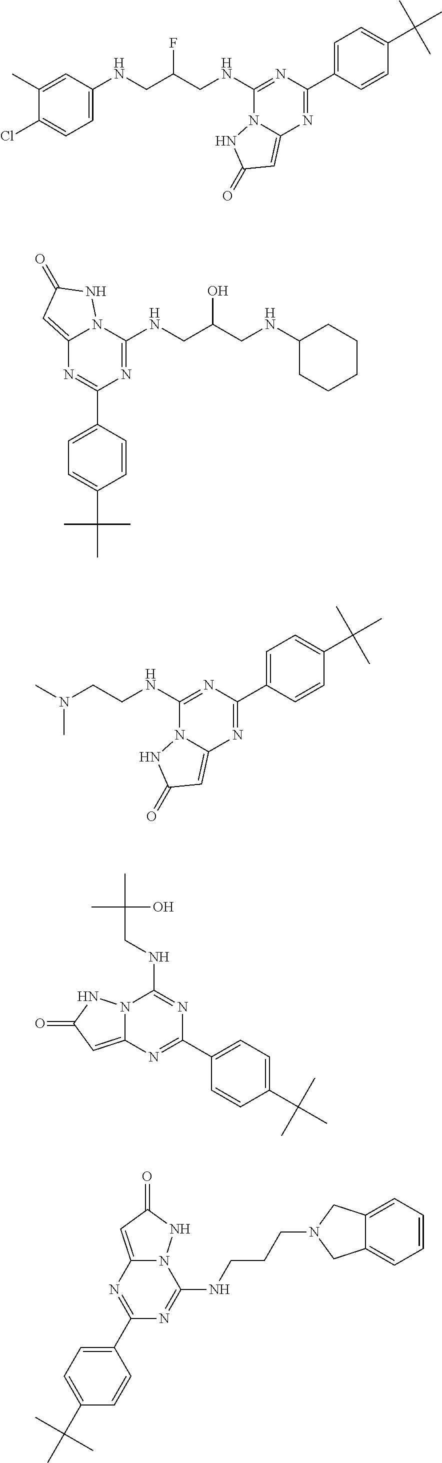

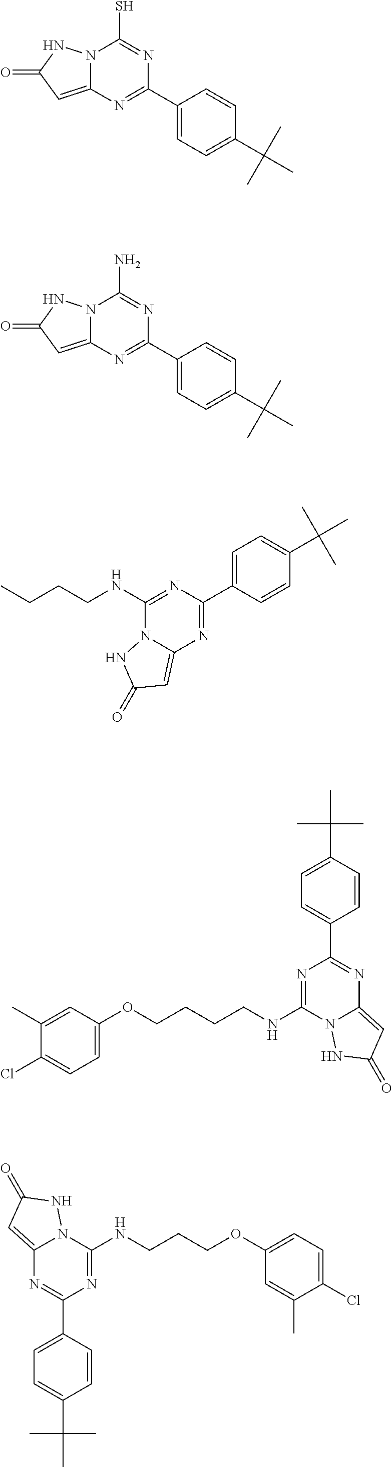

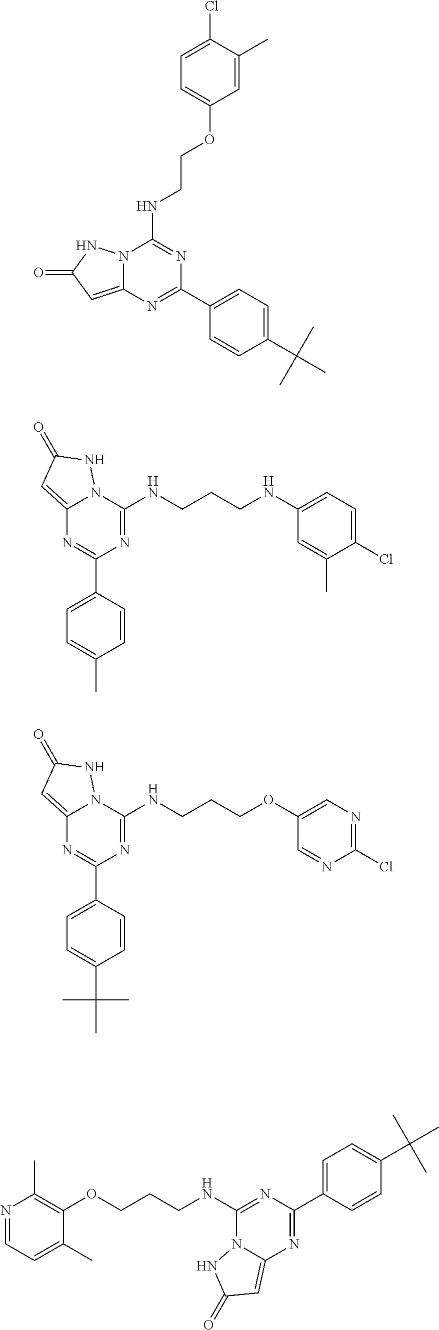 [SMILES: Cc1cc(OCCNc2nc(-c3ccc(C(C)(C)C)cc3)nc3cc(=O)[nH]n23)ccc1Cl ; Cc1ccc(-c2nc(NCCCNc3ccc(Cl)c(C)c3)n3[nH]c(=O)cc3n2)cc1 ; CC(C)(C)c1ccc(-c2nc(NCCCOc3cnc(Cl)nc3)n3[nH]c(=O)cc3n2)cc1 ; Cc1ccnc(C)c1OCCCNc1nc(-c2ccc(C(C)(C)C)cc2)nc2cc(=O)[nH]n12]

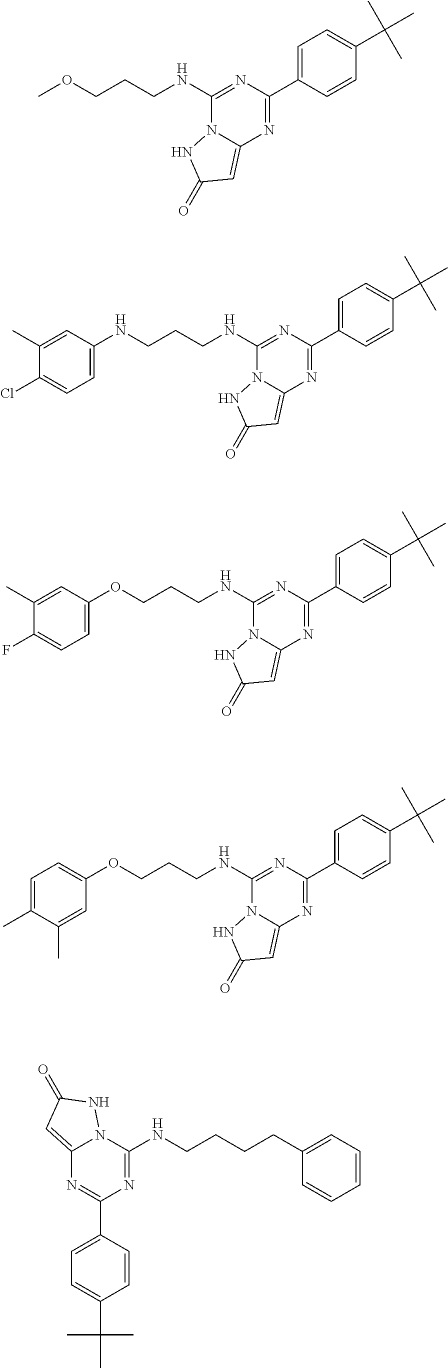

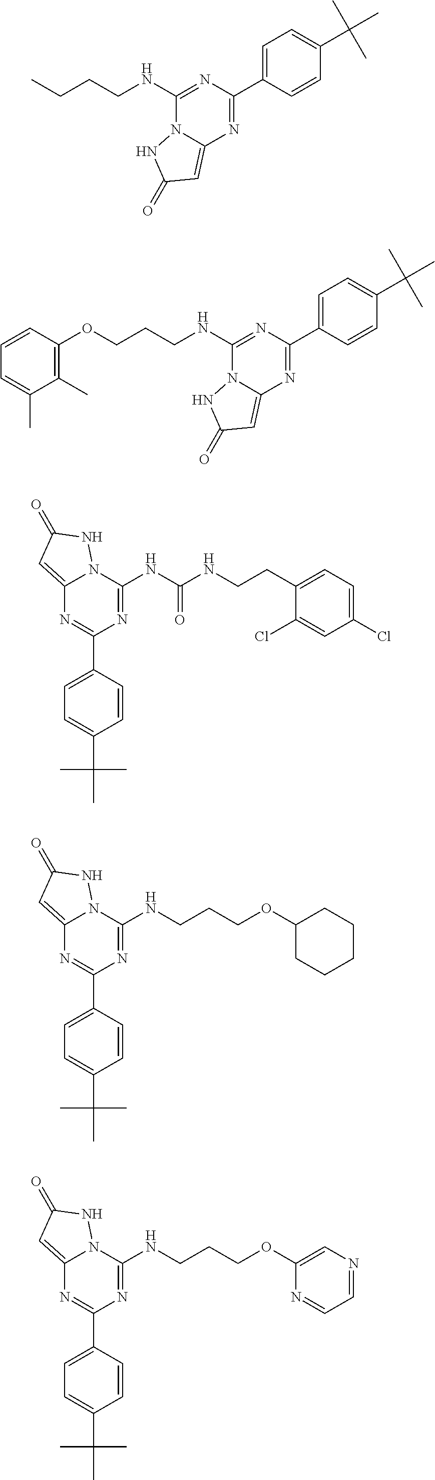 [SMILES: CCCCNc1nc(-c2ccc(C(C)(C)C)cc2)nc2cc(=O)[nH]n12 ; Cc1cccc(OCCCNc2nc(-c3ccc(C(C)(C)C)cc3)nc3cc(=O)[nH]n23)c1C ; CC(C)(C)c1ccc(-c2nc(NC(=O)NCCc3ccc(Cl)cc3Cl)n3[nH]c(=O)cc3n2)cc1 ; CC(C)(C)c1ccc(-c2nc(NCCCOC3CCCCC3)n3[nH]c(=O)cc3n2)cc1 ; CC(C)(C)c1ccc(-c2nc(NCCCOc3cnccn3)n3[nH]c(=O)cc3n2)cc1]

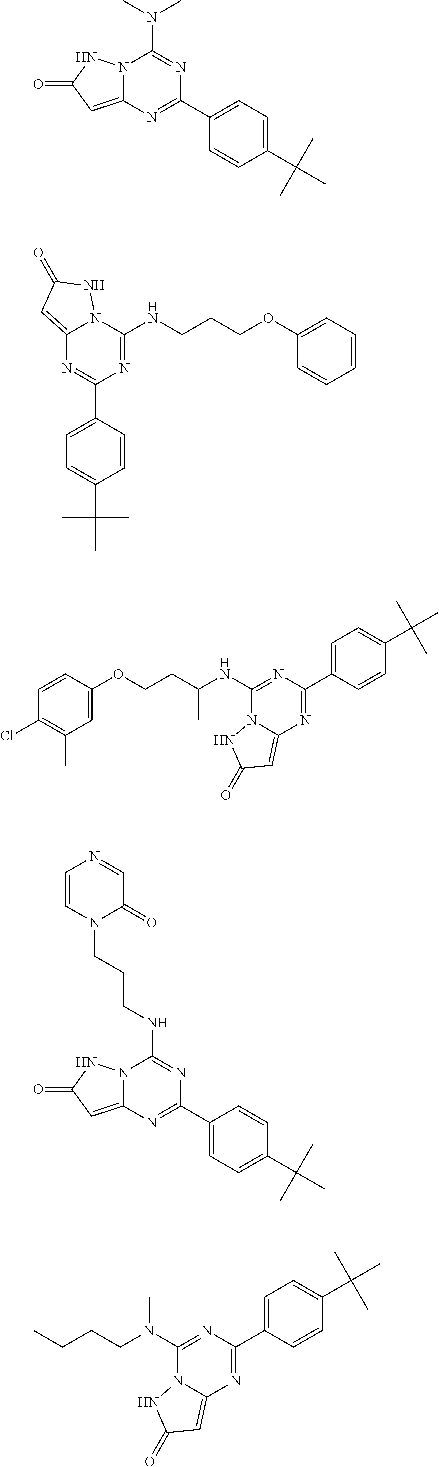

### Method of Synthesis

The present invention provides methods of preparing compounds of Formula (I′) and (IA). The present invention also provides methods of preparing compounds of Formula (I)-(VII).

Compounds of the structure S-IV can be prepared from S-III by substitution reaction with RX (R is optionally substituted alkyl such as benzyl, optionally substituted alkenyl such as allyl, or optionally substituted alkenyl heterocyclyl; LG is leaving group such as halogen). The general synthesis of compounds of Formula (S-III) from 2-cyanoethanoic acid hydrazide of Formula (S-II) has been described previously (M. R. H. Elmoghayar, S. O. Abdalla, M. Y. A.-S. Nasr, J. Heterocyclic Chem. 1984, 21, 781.)

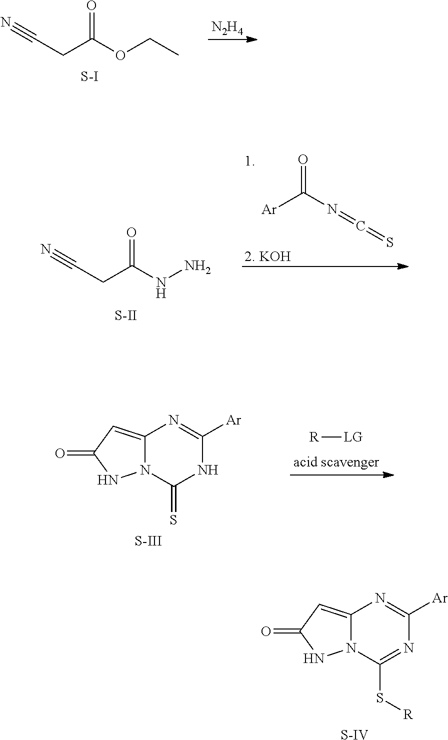

### Pharmaceutical Compositions

The present invention provides pharmaceutical compositions comprising a compound described herein, e.g., a compound of Formula (I′), Formula (IA) or Formulae (I)-(VII), or a pharmaceutically acceptable form thereof, as described herein, and a pharmaceutically acceptable excipient. The present invention also provides pharmaceutical compositions for use in treating and/or preventing a bacterial infection in a subject comprising a compound described herein, e.g., a compound of Formula (I′), Formula (IA), or Formulae (I)-(VII), or a pharmaceutically acceptable salt, solvate, hydrate, polymorph, co-crystal, tautomer, stereoisomer, isotopically labeled derivative, or prodrug thereof, and a pharmaceutically acceptable excipient. In certain embodiments, a provided composition comprises two or more compounds described herein. In certain embodiments, a compound described herein, or a pharmaceutically acceptable salt, solvate, hydrate, polymorph, co-crystal, tautomer, stereoisomer, isotopically labeled derivative, or prodrug thereof, is provided in an effective amount in the pharmaceutical composition. In certain embodiments, the effective amount is a therapeutically effective amount. In certain embodiments, the effective amount is an amount effective for inhibiting bacterial growth. In certain embodiments, the effective amount is an amount effective for killing bacteria. In certain embodiments, the bacterium which is the causative agent of the infection is a Gram-negative bacterium. In certain embodiments, the Gram-negative bacterium is selected from the group consisting of Escherichia, Citrobacter, Enterobacter, Klebsiella, Proteus, Serratia, Shigella, Salmonella, Morganella, Providencia, Edwardsiella, Erwinia, Hafnia, Yersinia, Acinetobacter, Vibrio, Aeromonas, Pseudomonas, Haemophilus, Pasteurella, Campylobacter, Helicobacter, Branhamella, Moraxella, Neisseria, Veillonella, Fusobacterium, Bacteroides, Actinobacillus, Aggregatibacter, Agrobacterium, Porphyromonas, Prevotella, Ruminobacter, Roseburia, Caulobacter, Francisella, Borrelia, Treponema, Brucella, and Rickettsia. In certain embodiments, the Gram-negative bacterium is selected from the group consisting of Escherichia coli, Morganella morganii, Branhamella catarrhalis, Veillonella parvula, Actinobacillus actinomycetemcomitans, Aggregatibacter actinomycetemcomitans, Caulobacter crescentus, and Treponema pallidum. In certain embodiments, the bacterium is a Gram-positive bacterium. In certain embodiments, the bacterium is at least one selected from the group consisting of Staphylococcus sp., Enterococcus sp., Escherichia coli, Bacillus sp., Salmonella sp., and Mycobacterium sp. In certain embodiments, the Gram-positive bacterium is selected from the group consisting of Staphylococcus, Streptococcus, Micrococcus, Peptococcus, Peptostreptococcus, Enterococcus, Bacillus, Clostridium, Lactobacillus, Listeria, Erysipelothrix, Propionibacterium, Eubacterium, Corynebacterium, Capnocytophaga, Bifidobacterium, and Gardnerella.

In certain embodiments, the bacterium is a drug-resistant bacterium. In certain embodiments the bacterium is selected from the group consisting of methicillin-resistant Staphylococcus aureus (MRSA), methicillin-resistant Staphylococcus epidermidis (MRSE), penicillin-resistant Streptococcus pneumonia, quinolone-resistant Staphylococcus aureus (QRSA), vancomycin-resistant Staphylococcus aureus (VRSA), vancomycin-resistant Enterococci (VRE), or multi-drug resistant Mycobacterium tuberculosis (MDR-TB).

Pharmaceutically acceptable excipients include any and all solvents, diluents, or other liquid vehicles, dispersions, suspension aids, surface active agents, isotonic agents, thickening or emulsifying agents, preservatives, solid binders, lubricants, and the like, as suited to the particular dosage form desired. General considerations in formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Pharmaceutical compositions described herein can be prepared by any method known in the art of pharmacology. In general, such preparatory methods include the steps of bringing the compound of the present invention (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween 20), polyoxyethylene sorbitan (Tween 60), polyoxyethylene sorbitan monooleate (Tween 80), sorbitan monopalmitate (Span 40), sorbitan monostearate (Span 60], sorbitan tristearate (Span 65), glyceryl monooleate, sorbitan monooleate (Span 80)), polyoxyethylene esters (e.g., polyoxyethylene monostearate (Myrj 45), polyoxyethylene hydrogenated castor oil, polyethoxylated castor oil, polyoxymethylene stearate, and Solutol), sucrose fatty acid esters, polyethylene glycol fatty acid esters (e.g., Cremophor™), polyoxyethylene ethers, (e.g., polyoxyethylene lauryl ether (Brij 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F68, Poloxamer 188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophorrm, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension or emulsion in a nontoxic parenterally acceptable diluent or solvent, for example, as a solution in 1,3-butanediol. Among the acceptable vehicles and solvents that can be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the conjugates of this invention with suitable non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets, and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes.

Dosage forms for topical and/or transdermal administration of a compound of this invention may include ointments, pastes, creams, lotions, gels, powders, solutions, sprays, inhalants and/or patches. Generally, the active ingredient is admixed under sterile conditions with a pharmaceutically acceptable carrier and/or any needed preservatives and/or buffers as can be required. Additionally, the present invention contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Jet injection devices which deliver liquid vaccines to the dermis via a liquid jet injector and/or via a needle which pierces the stratum corneum and produces a jet which reaches the dermis are suitable. Ballistic powder/particle delivery devices which use compressed gas to accelerate vaccine in powder form through the outer layers of the skin to the dermis are suitable. Alternatively or additionally, conventional syringes can be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition of the invention can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant can be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions of the invention formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition of the invention. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) and as much as 100% (w/w) of the active ingredient, and may comprise one or more of the additional ingredients described herein. A pharmaceutical composition of the invention can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition of the invention can be prepared, packaged, and/or sold in a formulation for ophthalmic administration. Such formulations may, for example, be in the form of eye drops including, for example, a 0.1/1.0% (w/w) solution and/or suspension of the active ingredient in an aqueous or oily liquid carrier. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are contemplated as being within the scope of this invention.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease, disorder, or condition being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the invention may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

Also encompassed by the invention are kits (e.g., pharmaceutical packs) to treat or prevent bacterial infections. The kits provided may comprise an inventive pharmaceutical composition or compound and a container (e.g., a vial, ampule, bottle, syringe, and/or dispenser package, or other suitable container). The kits provided may comprise an additional therapeutically active agents include, but are not limited to, antibiotics, anti-viral agents, anesthetics, anti-coagulants, inhibitors of an enzyme, steroidal agents, steroidal or non-steroidal anti-inflammatory agents, antihistamine, immunosuppressant agents, antigens, vaccines, antibodies, decongestant, sedatives, opioids, pain-relieving agents, analgesics, anti-pyretics, hormones, and prostaglandins, etc. In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one unit dosage form.

### Method of Use and Treatment

The present invention provides compounds and pharmaceutical compositions useful for inhibiting bacterial growth. The present invention provides compounds and pharmaceutical compositions useful for killing bacteria. In one aspect, the present invention provides methods for inhibiting bacterial growth or killing bacteria comprising administering an effective amount of a compound described herein (e.g., a compound of Formula (I′), Formula (IA), or Formulae (I)-(VII)), or a pharmaceutically acceptable salt, solvate, hydrate, polymorph, co-crystal, tautomer, stereoisomer, isotopically labeled derivative, or prodrug thereof), to a subject in need of treatment. In another aspect, the present invention provides methods for treating or preventing bacterial infection comprising administering an effective amount of a compound described herein (e.g., a compound of Formula (I′), Formula (IA), or Formulae (I)-(VII)), or a pharmaceutically acceptable salt, solvate, hydrate, polymorph, co-crystal, tautomer, stereoisomer, isotopically labeled derivative, or prodrug thereof), to a subject in need of treatment. In certain embodiments, the compound is not of the formula:

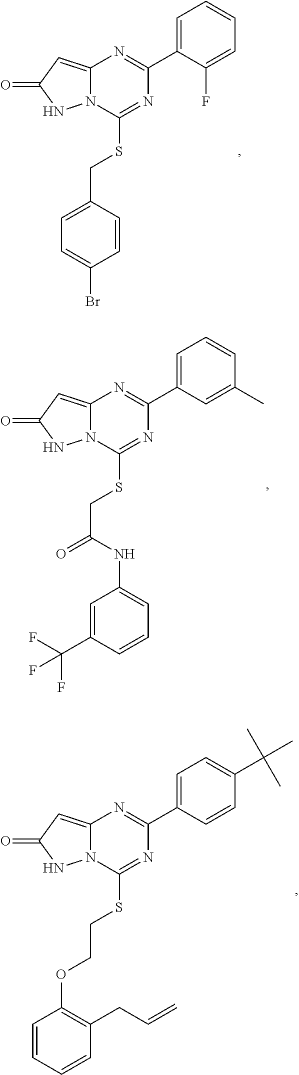 [SMILES: O=c1cc2nc(-c3ccccc3F)nc(SCc3ccc(Br)cc3)n2[nH]1 ; Cc1cccc(-c2nc(SCC(=O)Nc3cccc(C(F)(F)F)c3)n3[nH]c(=O)cc3n2)c1 ; C=CCc1ccccc1OCCSc1nc(-c2ccc(C(C)(C)C)cc2)nc2cc(=O)[nH]n12]

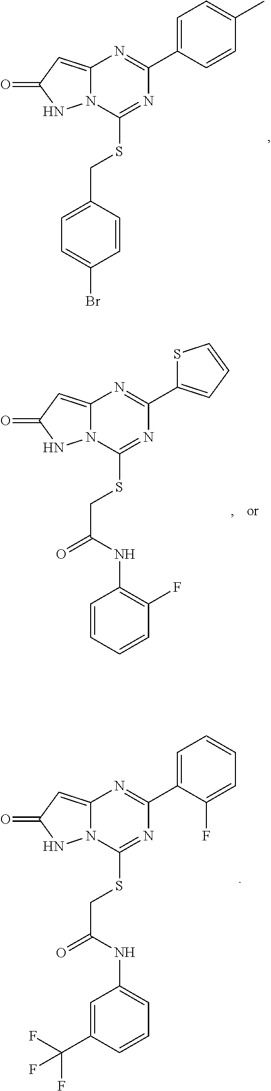 [SMILES: Cc1ccc(-c2nc(SCc3ccc(Br)cc3)n3[nH]c(=O)cc3n2)cc1 ; O=C(CSc1nc(-c2cccs2)nc2cc(=O)[nH]n12)Nc1ccccc1F ; O=C(CSc1nc(-c2ccccc2F)nc2cc(=O)[nH]n12)Nc1cccc(C(F)(F)F)c1]

In certain embodiments, the effective amount is a therapeutically effective amount. In certain embodiments, the effective amount is a prophylactically effective amount. In certain embodiments, the subject is suffering from a bacterial infection. In certain embodiments, the subject is susceptible to having a bacterial infection. In certain embodiments, the subject has been exposed or is at risk of being exposed to a pathogenic microorganism. The infection may be prevented or at least the chances of infection may be reduced by the administration of a prophylactic amount of a compound described herein.

In yet another aspect, provided is a method of treating or preventing a bacterial infection caused by bacteria that are resistant to other treatments. In certain embodiments, provided is a method of treating or preventing a bacterial infection caused by bacteria that are multi-drug tolerant. In certain embodiments, provided is a method of treating or preventing a bacterial infection caused by bacteria that are multi-drug resistant. In certain embodiments, provided is a method of treating or preventing a bacterial infection caused by bacteria that neither grow nor die in the presence other treatments. In certain embodiments, provided is a method of treating or preventing a bacterial infection caused by bacteria that neither grow nor die as a result of other treatments. In certain embodiments, provided methods can be conducted in vivo (i.e., by administration to a subject). For example, in certain embodiments, provided is a method of treating and/or preventing a bacterial infection comprising administering an effective amount of a compound of the present invention, e.g., a compound of Formula (I′), Formula (IA), or Formulae (I)-(VII), or a pharmaceutically acceptable salt, solvate, hydrate, polymorph, co-crystal, tautomer, stereoisomer, isotopically labeled derivative, or prodrug thereof, to a subject with a bacterial infection or at risk of developing a bacterial infection.

For example, in certain embodiments, provided is a method of treating a microbial infection comprising contacting an effective amount of the compound of the present invention with a microorganism. In certain embodiments, provided is an in vitro method of treating microbial infection comprising contacting an effective amount of the compound of the present invention with a microorganism in a cell culture. In certain embodiments, provided is an in vivo method of treating microbial infection comprising administering an effective amount of the compound of the present invention to a subject with a microbial infection. In certain embodiments, the microorganism is a bacterium.

In another aspect, the present invention provides a method of killing bacteria in a subject comprising administering an effective amount of a compound described herein, or a pharmaceutically-acceptable salt, solvate, hydrate, polymorph, co-crystal, tautomer, stereoisomer, isotopically labeled derivative, or prodrug thereof. In certain embodiments, the compounds described herein inhibit bacterial cell wall biosynthesis. In certain embodiments, the compounds described herein inhibit the synthesis of peptidoglycan (PG), the polysaccharide strands containing peptide cross bridges in the bacterial cell walls. In certain embodiments, the compounds described herein inhibit the cross-linking of polysaccharide strands. In certain embodiments, the compounds described herein cause bacterial death by inhibiting the synthesis of peptidoglycan (PG). In certain embodiments, the compounds described herein cause bacterial death by inhibiting the synthesis of polysaccharide strands in the bacteria cell wall, where the polysaccharide strands are formed by peptidoglycan glycosyltransferases (PGTs).

The present invention provides use of a compound of the present invention, e.g., a compound of Formula (I′), Formula (IA), or Formulae (I)-(VII), or a pharmaceutically acceptable salt, solvate, hydrate, polymorph, co-crystal, tautomer, stereoisomer, isotopically labeled derivative, or prodrug thereof, for the manufacture of a medicament for use in treating and/or preventing a bacterial infection in a subject in need thereof. In certain embodiments, the present invention provides a compound of the present invention, e.g., a compound of Formula (I′), Formula (IA), or Formulae (I)-(VII), or a pharmaceutically acceptable salt, solvate, hydrate, polymorph, co-crystal, tautomer, stereoisomer, isotopically labeled derivative, or prodrug thereof, for use in treating and/or preventing a bacterial infection in a subject in need thereof.

In certain embodiments, the bacterial infection being treated or prevented is an infection with a Gram-positive bacteria. Exemplary Gram-positive bacteria include, but are not limited to, Staphylococcus, Streptococcus, Micrococcus, Peptococcus, Peptostreptococcus, Enterococcus, Bacillus, Clostridium, Lactobacillus, Listeria, Erysipelothrix, Propionibacterium, Eubacterium, Corynebacterium, Capnocytophaga, Bifidobacterium, and Gardnerella. Exemplary Gram-positive bacteria include, but are not limited to, Staphylococcus, Streptococcus, Micrococcus, Peptococcus, Peptostreptococcus, Enterococcus, Bacillus, Clostridium, Lactobacillus, Listeria, Erysipelothrix, Propionibacterium, Eubacterium, and Corynebacterium. In certain embodiments, the Gram-positive bacteria is a bacteria of the phylum Firmicutes. In certain embodiments, the bacteria is a member of the phylum Firmicutes and the genus Enterococcus, i.e., the bacterial infection is an Enterococcus infection. Exemplary Enterococci bacteria include, but are not limited to, E. avium, E. durans, E. faecalis, E. faecium, E. gallinarum, E. solitarius, E. casseliflavus, and E. raffinosus. In certain embodiments, the Enterococcus infection is an E. faecalis infection. In certain embodiments, the Enterococcus infection is an E. faecium infection. In certain embodiments, the bacteria is a member of the phylum Firmicutes and the genus Staphylococcus, i.e., the bacterial infection is a Staphylococcus infection. Exemplary Staphylococci bacteria include, but are not limited to, S. arlettae, S. aureus, S. auricularis, S. capitis, S. caprae, S. carnous, S. chromogenes, S. cohii, S. condimenti, S. croceolyticus, S. delphini, S. devriesei, S. epidermis, S. equorum, S. felis, S. fluroettii, S. gallinarum, S. haemolyticus, S. hominis, S. hyicus, S. intermedius, S. kloosii, S. leei, S. lenus, S. lugdunesis, S. lutrae, S. lyticans, S. massiliensis, S. microti, S. muscae, S. nepalensis, S. pasteuri, S. penttenkoferi, S. piscifermentans, S. psuedointermedius, S. psudolugdensis, S. pulvereri, S. rostri, S. saccharolyticus, S. saprophyticus, S. schleiferi, S. sciuri, S. simiae, S. simulans, S. stepanovicii, S. succinus, S. vitulinus, S. warneri, and S. xylosus. In certain embodiments, the Staphylococcus infection is an S. aureus infection. In certain embodiments, the Staphylococcus infection is an S. epidermis infection.

In certain embodiments, the bacterial infection being treated or prevented is an infection with a Gram-negative bacteria. Exemplary Gram-negative bacteria include, but are not limited to, Escherichia, Citrobacter, Enterobacter, Klebsiella, Proteus, Serratia, Shigella, Salmonella, Morganella, Providencia, Edwardsiella, Erwinia, Hafnia, Yersinia, Acinetobacter, Vibrio, Aeromonas, Pseudomonas, Haemophilus, Pasteurella, Campylobacter, Helicobacter, Branhamella, Moraxella, Neisseria, Veillonella, Fusobacterium, Bacteroides, Actinobacillus, Aggregatibacter, Agrobacterium, Porphyromonas, Prevotella, Ruminobacter, Roseburia, Caulobacter, Francisella, Borrelia, Treponema, Brucella, and Rickettsia. In certain embodiments, the bacterium is selected from the group consisting of Escherichia coli, Morganella morganii, Branhamella catarrhalis, Veillonella parvula, Actinobacillus actinomycetemcomitans, Aggregatibacter actinomycetemcomitans, Caulobacter crescentus, and Treponema pallidum. Exemplary Gram-negative bacteria include, but are not limited to, Escherichia coli, Caulobacter crescentus, Pseudomonas aeruginosa, Agrobacterium tumefaciens, Branhamella catarrhalis, Citrobacter diversus, Enterobacter aerogenes, Klebsiella pneumoniae, Proteus mirabilis, Salmonella typhimurium, Neisseria meningitidis, Serratia marcescens, Shigella sonnei, Neisseria gonorrhoeae, Acinetobacter baumannii, Salmonella enteriditis, Fusobacterium nucleatum, Veillonella parvula, Bacteroides forsythus, Actinobacillus actinomycetemcomitans, Aggregatibacter actinomycetemcomitans, Porphyromonas gingivalis, Helicobacter pylori, Francisella tularensis, Yersinia pestis, Morganella morganii, Edwardsiella tarda, Acinetobacter baumannii and Haemophilus influenzae. In certain embodiments, the Gram-negative bacteria species is Escherichia coli, Bacillus sp., Salmonella sp., and Mycobacterium sp.

In certain embodiments, the bacterial infection is resistant to other antibiotic therapy. For example, in certain embodiments, the bacterial infection is vancomycin resistant (VR). In certain embodiments, the bacterial infection is a vancomycin-resistant E. faecalis infection. In certain embodiments, the bacterial infection is a vancomycin-resistant E. faecium infection. In certain embodiments, the bacterial infection is a vancomycin-resistant Staphylococcus aureus (VRSA) infection. In certain embodiments, the bacterial infection is a vancomycin-resistant Enterococci (VRE) infection. In certain embodiments, the bacterial infection is methicillin-resistant (MR). In certain embodiments, the bacterial infection is a methicillin-resistant S. aureus (MRSA) infection. In certain embodiments, the bacterial infection is methicillin-resistant Staphylococcus epidermidis (MRSE) infection. In certain embodiments, the bacterial infection is a penicillin-resistant Streptococcus pneumonia infection. In certain embodiments, the bacterial infection is a quinolone-resistant Staphylococcus aureus (QRSA) infection. In certain embodiments, the bacterial infection is multi-drug resistant Mycobacterium tuberculosis infection.

In another aspect, the compounds of the present invention induce hypersusceptibility of Gram-negative bacteria to the provided compounds. In certain embodiments, the present invention provides a method of treating and/or preventing a Gram-negative infection with a combination of at least one compound provided herein and another antibiotic. In certain embodiments, the additional antibiotic is typically inactive against Gram-negative bacteria.

In another aspect, the compounds of the present invention inhibit the growth of or kill rapidly dividing cells such as stimulated inflammatory cells. Thus, the present invention also contemplates the treatment of a disease, disorder, or condition associated with abnormal cellular proliferation, such as cancer, autoimmune diseases, inflammatory diseases, and diabetic retinopathy.

Thus, in one aspect, provided is a method of treating cancer comprising administering an effective amount of the compound of the present invention or a pharmaceutically acceptable salt, solvate, hydrate, polymorph, co-crystal, tautomer, stereoisomer, isotopically labeled derivative, or prodrug thereof to a subject.

In another aspect, provided is a method of treating an autoimmune disease comprising administering an effective amount of the compound of the present invention or a pharmaceutically acceptable salt, solvate, hydrate, polymorph, co-crystal, tautomer, stereoisomer, isotopically labeled derivative, or prodrug thereof to a subject.

In yet another aspect, provided is a method of treating an inflammatory disease comprising administering the compound of the present invention or a pharmaceutically acceptable salt, solvate, hydrate, polymorph, co-crystal, tautomer, stereoisomer, isotopically labeled derivative, or prodrug thereof to a subject.

In yet another aspect, provided is a method of treating diabetic retinopathy comprising administering an effective amount of the compound of the present invention or a pharmaceutically acceptable salt, solvate, hydrate, polymorph, co-crystal, tautomer, stereoisomer, isotopically labeled derivative, or prodrug thereof to a subject.

Compounds provided herein are typically formulated in dosage unit form for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease, disorder, or condition being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general the most appropriate route of administration will depend upon a variety of factors including the nature of the agent (e.g., its stability in the environment of the gastrointestinal tract), the condition of the subject (e.g., whether the subject is able to tolerate oral administration), etc.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional therapeutically active agents. The compounds or compositions can be administered in combination with additional therapeutically active agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional therapeutically active agents. In general, each agent will be administered at a dose and/or on a time schedule determined for that agent. In will further be appreciated that the additional therapeutically active agent utilized in this combination can be administered together in a single composition or administered separately in different compositions. The particular combination to employ in a regimen will take into account compatibility of the inventive compound with the additional therapeutically active agent and/or the desired therapeutic effect to be achieved. In general, it is expected that additional therapeutically active agents utilized in combination be utilized at levels that do not exceed the levels at which they are utilized individually. In some embodiments, the levels utilized in combination will be lower than those utilized individually.

Exemplary additional therapeutically active agents include, but are not limited to, antibiotics, anti-viral agents, anesthetics, anti-coagulants, inhibitors of an enzyme, steroidal agents, steroidal or non-steroidal anti-inflammatory agents, antihistamine, immunosuppressant agents, antigens, vaccines, antibodies, decongestant, sedatives, opioids, pain-relieving agents, analgesics, anti-pyretics, hormones, and prostaglandins, etc. Therapeutically active agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells.

In certain embodiments, the additional therapeutically agent is an antibiotic. Exemplary antibiotics include, but are not limited to, penicillins (e.g., penicillin, amoxicillin), cephalosporins (e.g., cephalexin), macrolides (e.g., erythromycin, clarithormycin, azithromycin, troleandomycin), fluoroquinolones (e.g., ciprofloxacin, levofloxacin, ofloxacin), sulfonamides (e.g., co-trimoxazole, trimethoprim), tetracyclines (e.g., tetracycline, chlortetracycline, oxytetracycline, demeclocycline, methacycline, sancycline, doxycline, aureomycin, terramycin, minocycline, 6-deoxytetracycline, lymecycline, meclocycline, methacycline, rolitetracycline, and glycylcycline antibiotics (e.g., tigecycline)), aminoglycosides (e.g., gentamicin, tobramycin, paromomycin), aminocyclitol (e.g., spectinomycin), chloramphenicol, sparsomycin, quinupristin/dalfoprisin (Syndercid™),

In certain embodiments, the antibiotic is a ribosome-targeting antibiotic. Antibiotics target ribosomes at distinct locations within functionally relevant sites. They exert their inhibitory action by diverse modes, including competing with substrate binding, interfering with ribosomal dynamics, minimizing ribosomal mobility, facilitating miscoding, hampering the progression of the mRNA chain, and blocking the nascent protein exit tunnel. Examples of antibiotics that reveal novel ribosomal properties or enforced otherwise observed findings include the following: decoding (paromomycin); mRNA progression (spectinomycin); A-site binding to the small (tetracycline antibiotic) and the large (chloramphenicol) subunits; PTC mobility (sparsomycin); tRNA rotatory motion (quinupristin/dalfoprisin), and tunnel gating (troleandomycin); see Yonath, Annu. Rev. Biochem. (2005) 74:649-679.

In certain embodiments, the compound used in any of the methods as described herein include, but are not limited to compounds of Formulae (I′), (IA), and (I)-(VII). In certain embodiments, the compound used in any of the methods as described herein include, but are not limited to:

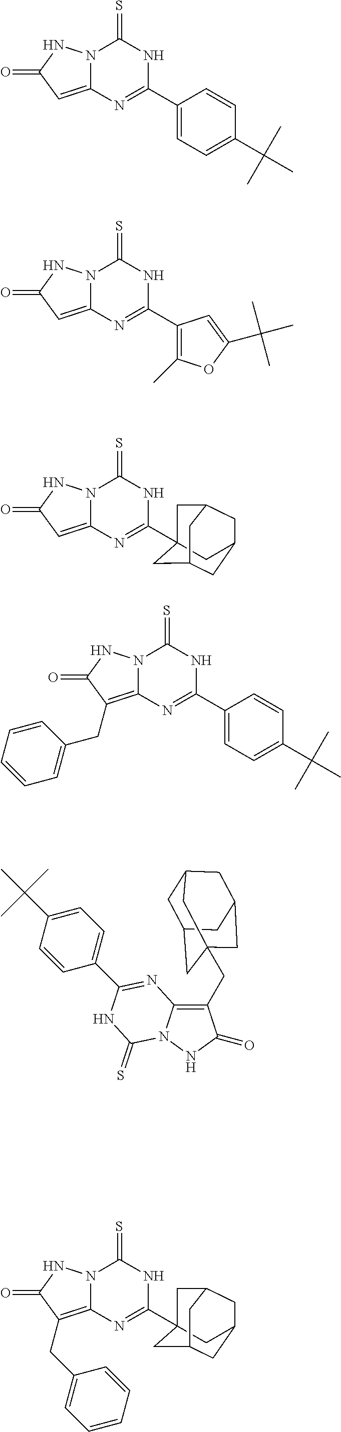

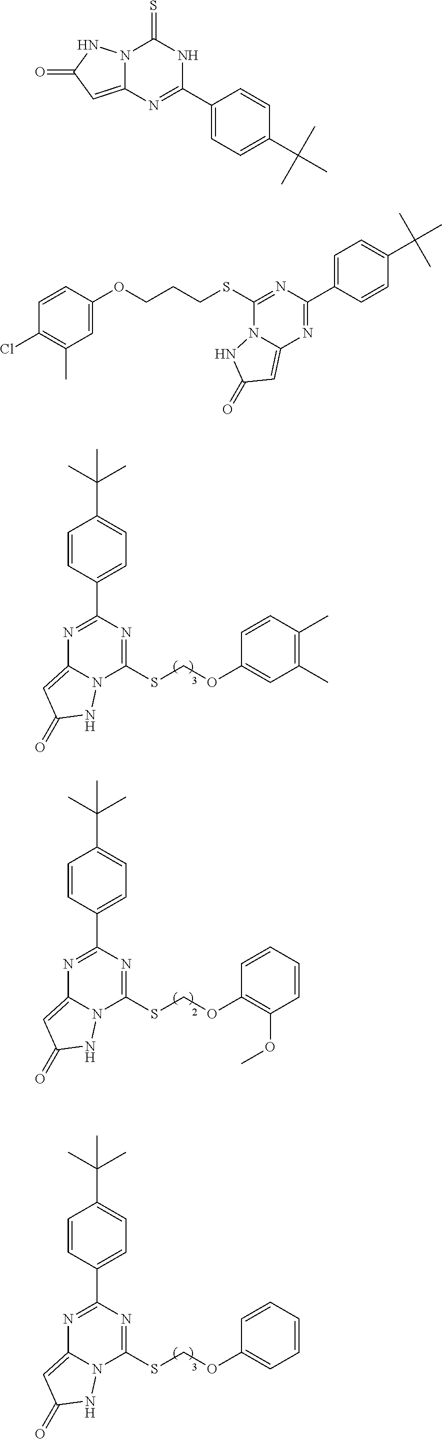

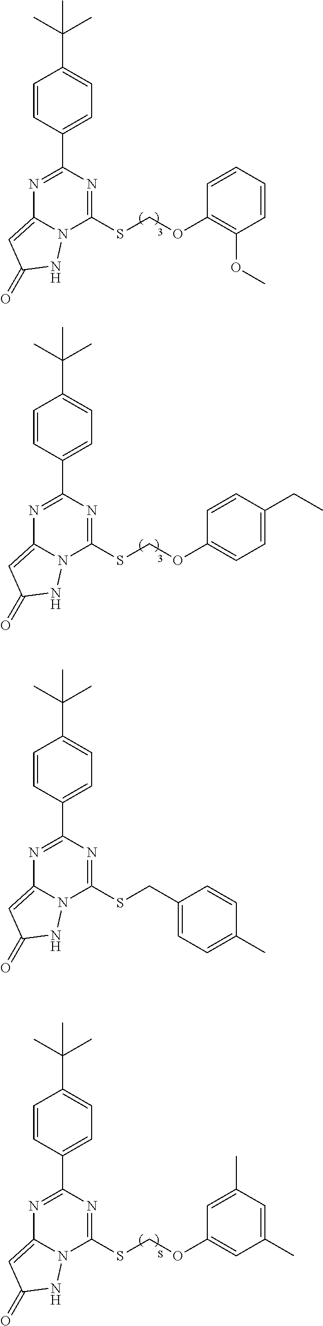

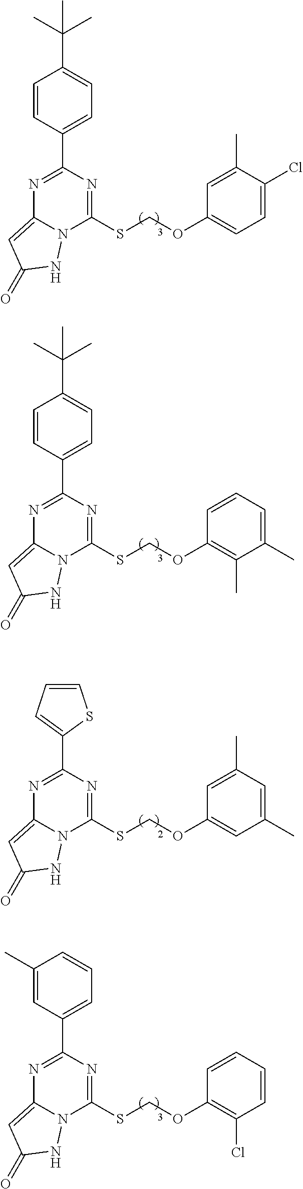

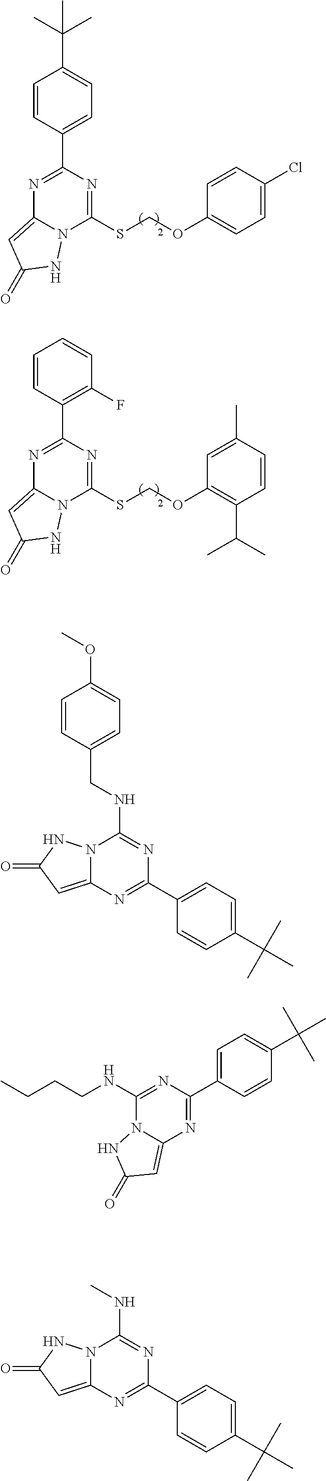

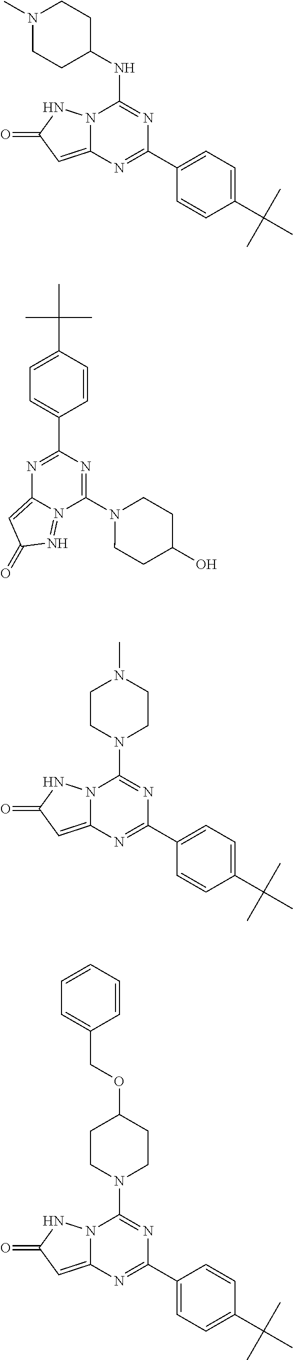

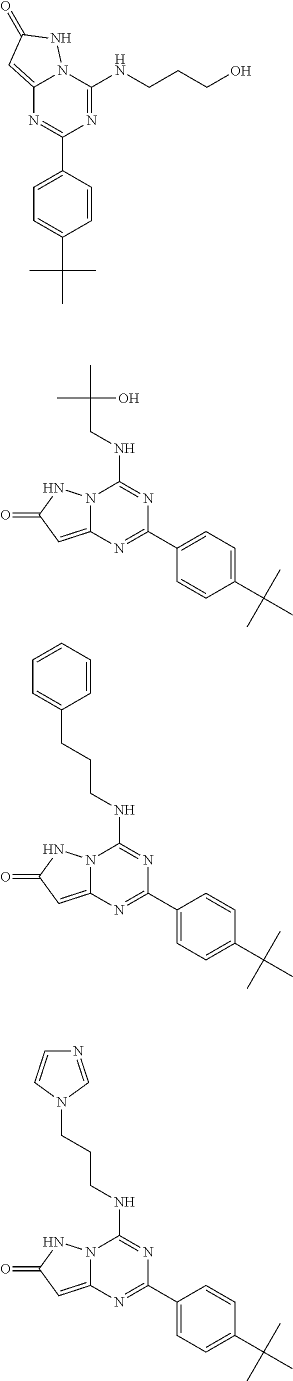 [SMILES: CC(C)(C)c1ccc(-c2nc(NCCCO)n3[nH]c(=O)cc3n2)cc1 ; CC(C)(O)CNc1nc(-c2ccc(C(C)(C)C)cc2)nc2cc(=O)[nH]n12 ; CC(C)(C)c1ccc(-c2nc(NCCCc3ccccc3)n3[nH]c(=O)cc3n2)cc1 ; CC(C)(C)c1ccc(-c2nc(NCCCn3ccnc3)n3[nH]c(=O)cc3n2)cc1]

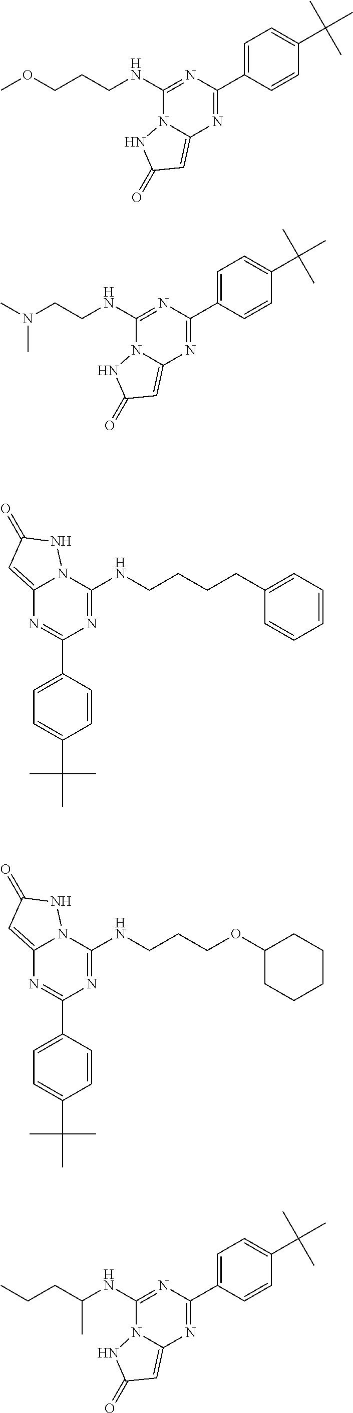

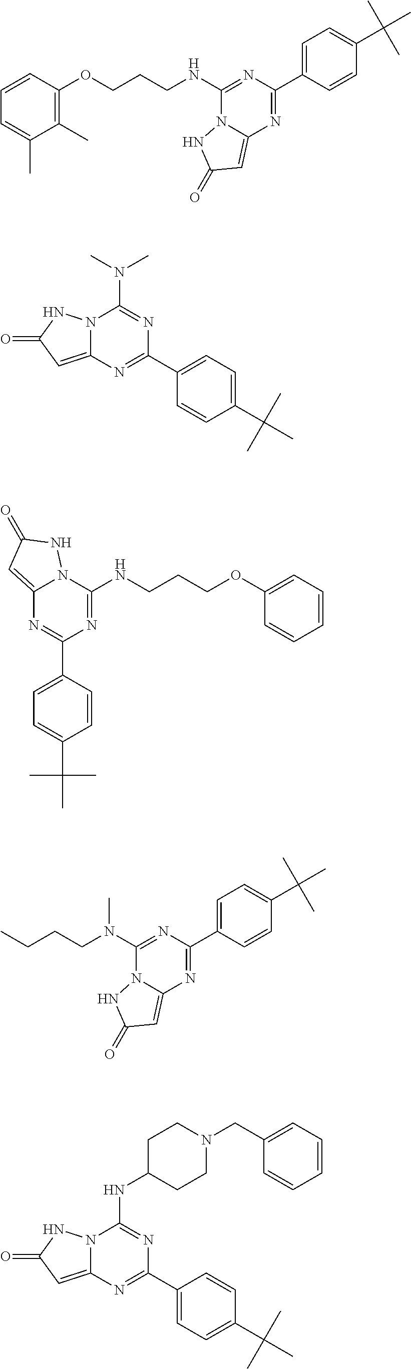

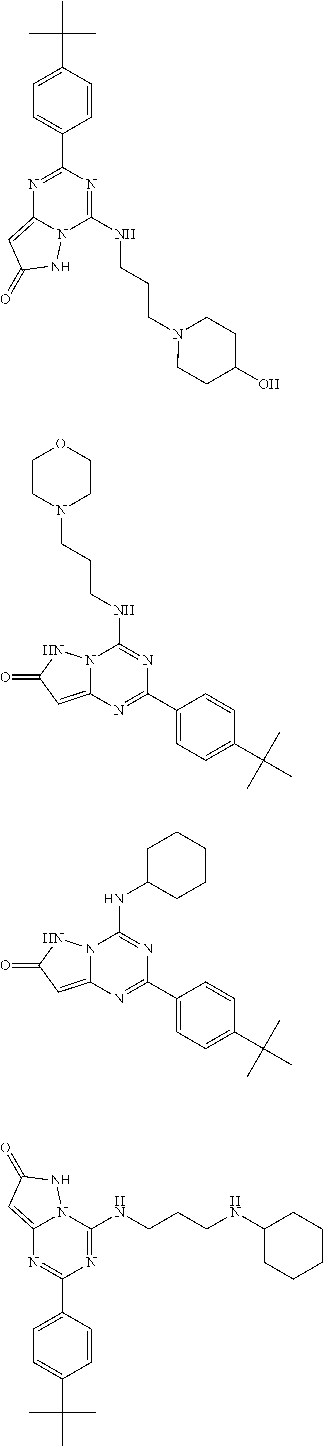 [SMILES: CC(C)(C)c1ccc(-c2nc(NCCCN3CCC(O)CC3)n3[nH]c(=O)cc3n2)cc1 ; CC(C)(C)c1ccc(-c2nc(NCCCN3CCOCC3)n3[nH]c(=O)cc3n2)cc1 ; CC(C)(C)c1ccc(-c2nc(NC3CCCCC3)n3[nH]c(=O)cc3n2)cc1 ; CC(C)(C)c1ccc(-c2nc(NCCCNC3CCCCC3)n3[nH]c(=O)cc3n2)cc1]

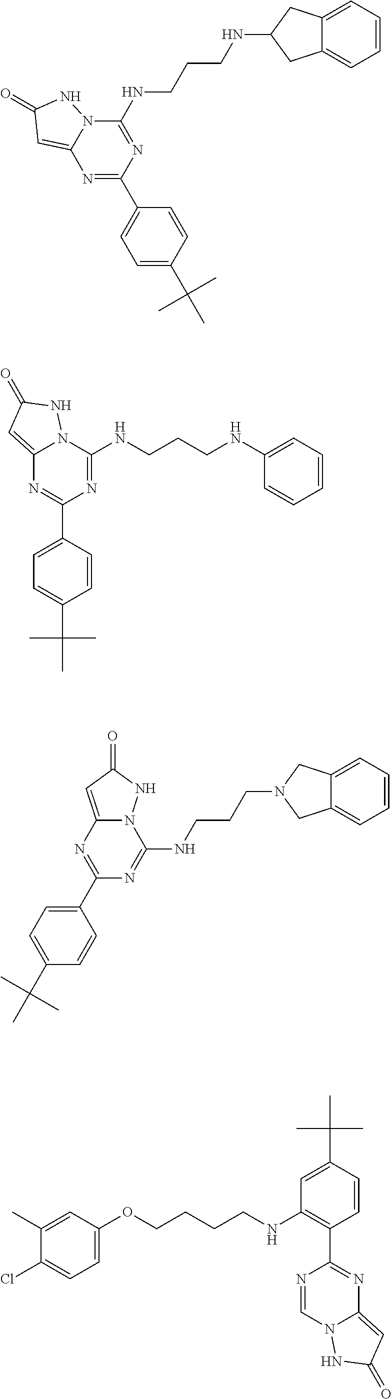 [SMILES: CC(C)(C)c1ccc(-c2nc(NCCCNC3Cc4ccccc4C3)n3[nH]c(=O)cc3n2)cc1 ; CC(C)(C)c1ccc(-c2nc(NCCCNc3ccccc3)n3[nH]c(=O)cc3n2)cc1 ; CC(C)(C)c1ccc(-c2nc(NCCCN3Cc4ccccc4C3)n3[nH]c(=O)cc3n2)cc1 ; Cc1cc(OCCCCNc2cc(C(C)(C)C)ccc2-c2ncn3[nH]c(=O)cc3n2)ccc1Cl]

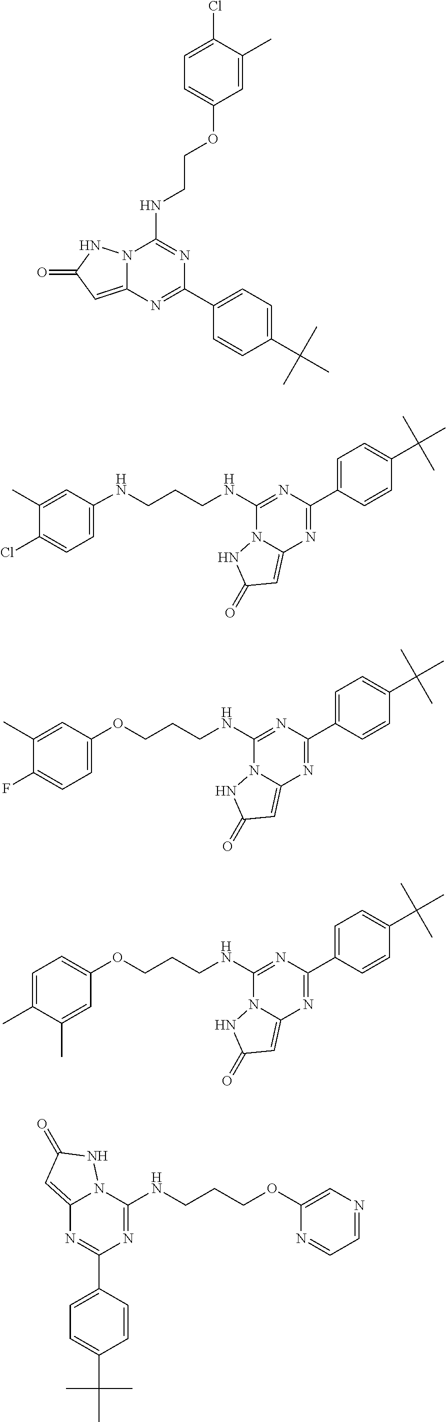

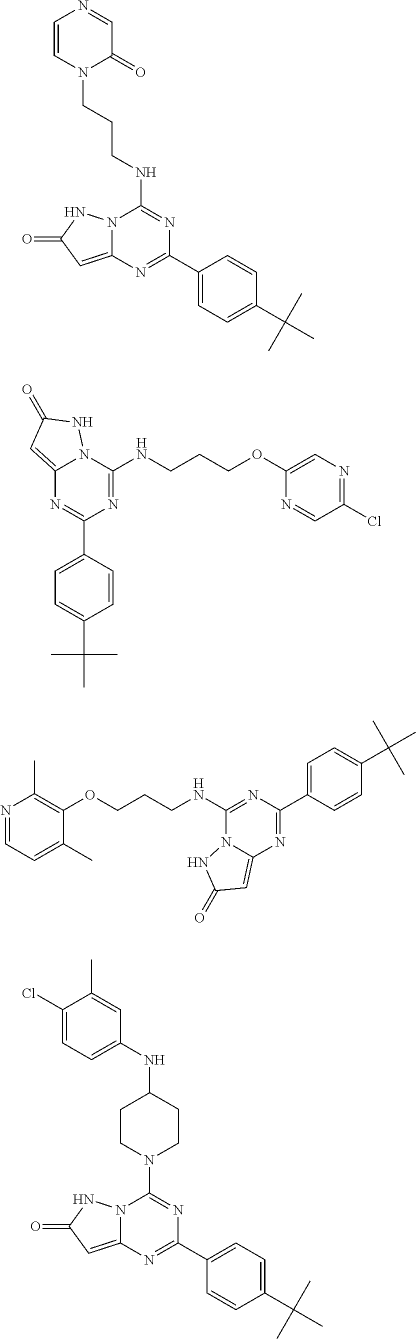 [SMILES: CC(C)(C)c1ccc(-c2nc(NCCCn3ccncc3=O)n3[nH]c(=O)cc3n2)cc1 ; CC(C)(C)c1ccc(-c2nc(NCCCOc3cnc(Cl)cn3)n3[nH]c(=O)cc3n2)cc1 ; Cc1ccnc(C)c1OCCCNc1nc(-c2ccc(C(C)(C)C)cc2)nc2cc(=O)[nH]n12 ; Cc1cc(NC2CCN(c3nc(-c4ccc(C(C)(C)C)cc4)nc4cc(=O)[nH]n34)CC2)ccc1Cl]

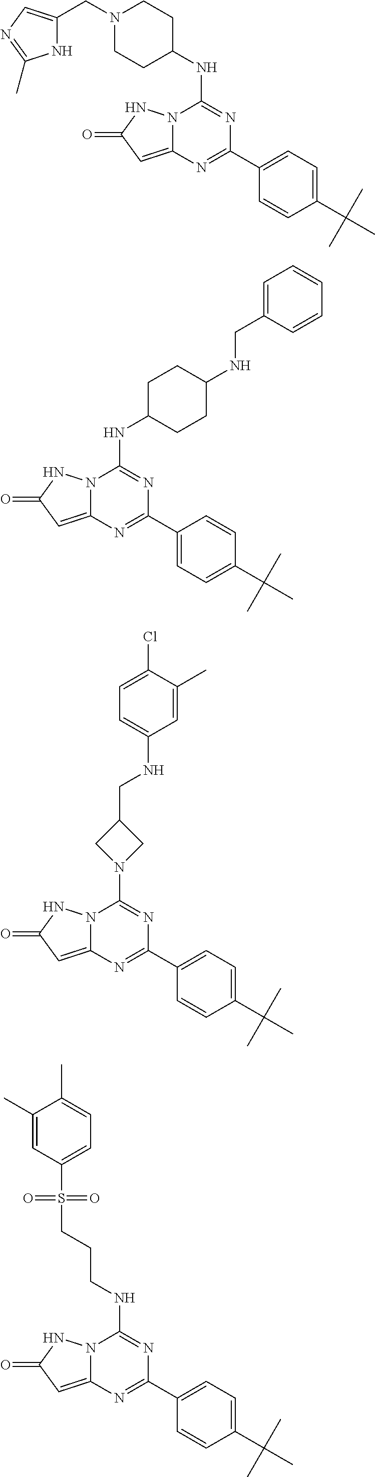

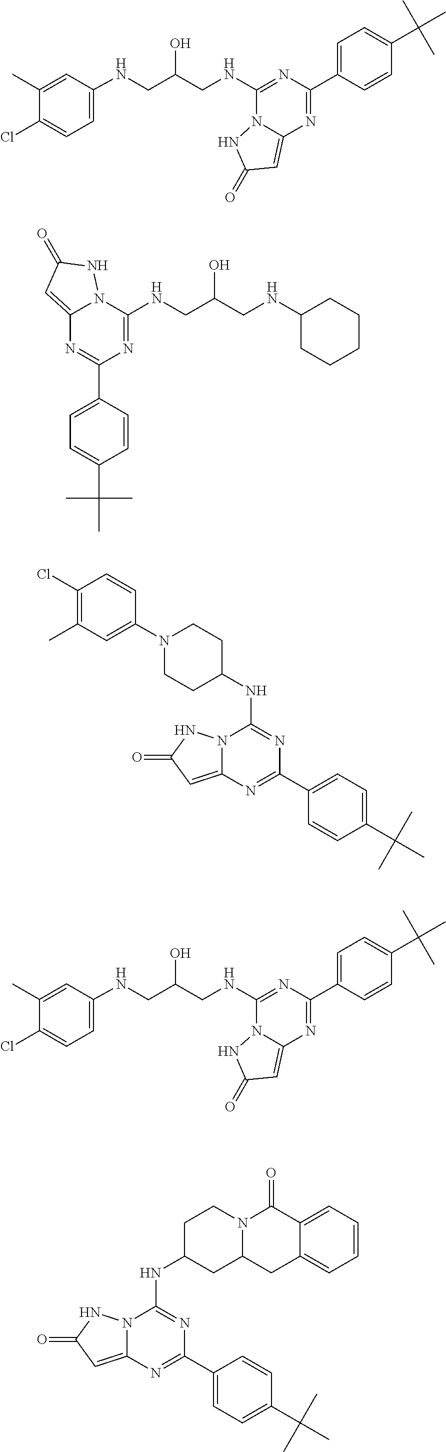

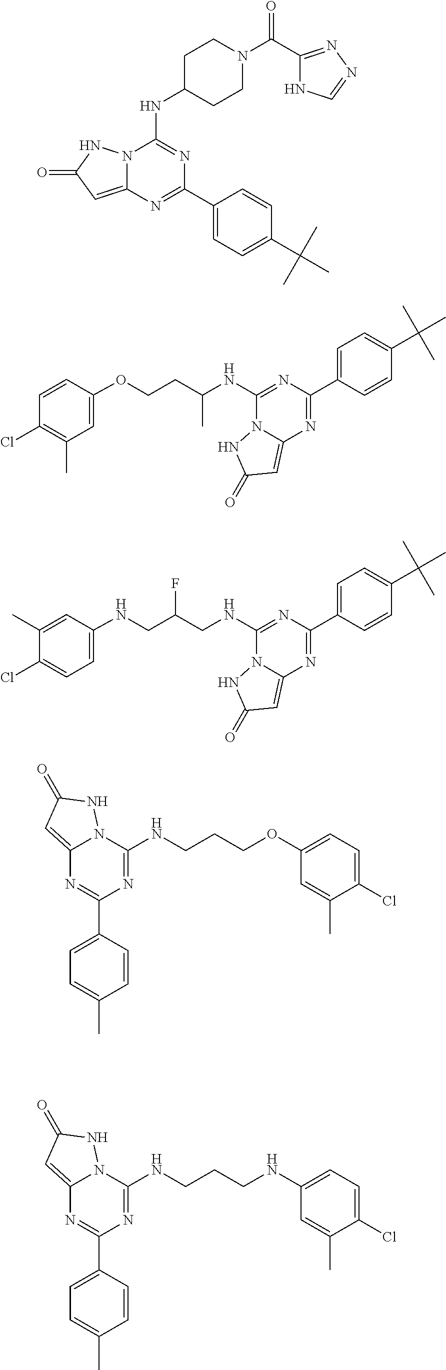 [SMILES: CC(C)(C)c1ccc(-c2nc(NC3CCN(C(=O)c4nnc[nH]4)CC3)n3[nH]c(=O)cc3n2)cc1 ; Cc1cc(OCCC(C)Nc2nc(-c3ccc(C(C)(C)C)cc3)nc3cc(=O)[nH]n23)ccc1Cl ; Cc1cc(NCC(F)CNc2nc(-c3ccc(C(C)(C)C)cc3)nc3cc(=O)[nH]n23)ccc1Cl ; Cc1ccc(-c2nc(NCCCOc3ccc(Cl)c(C)c3)n3[nH]c(=O)cc3n2)cc1 ; Cc1ccc(-c2nc(NCCCNc3ccc(Cl)c(C)c3)n3[nH]c(=O)cc3n2)cc1]

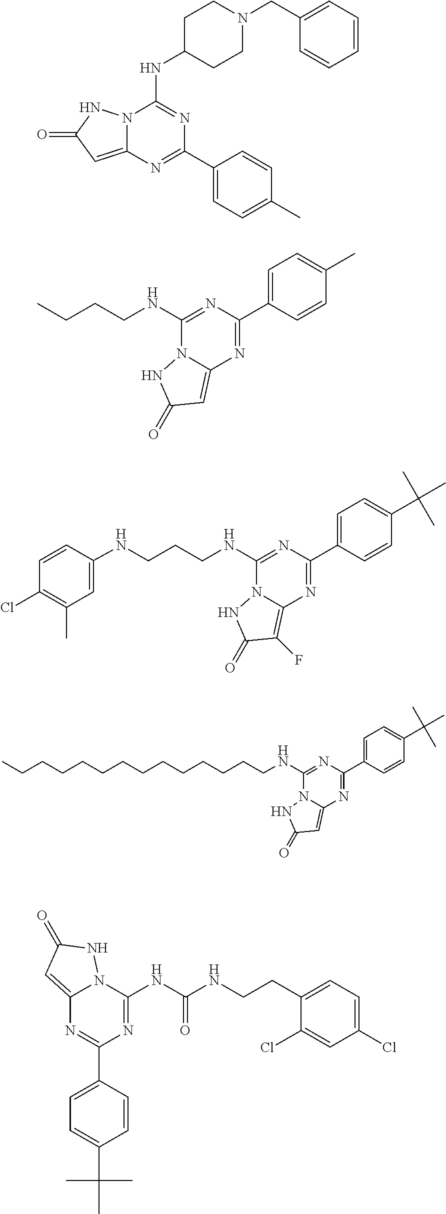

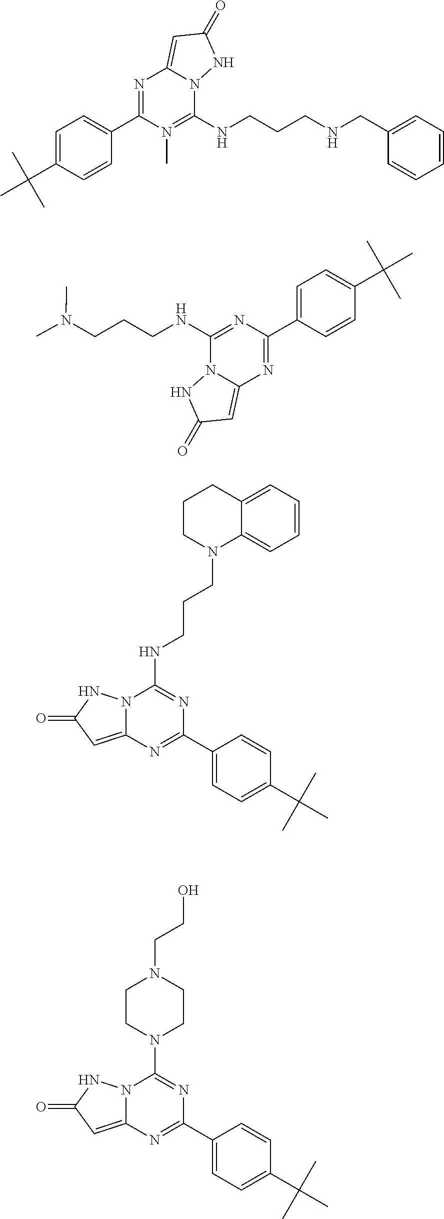

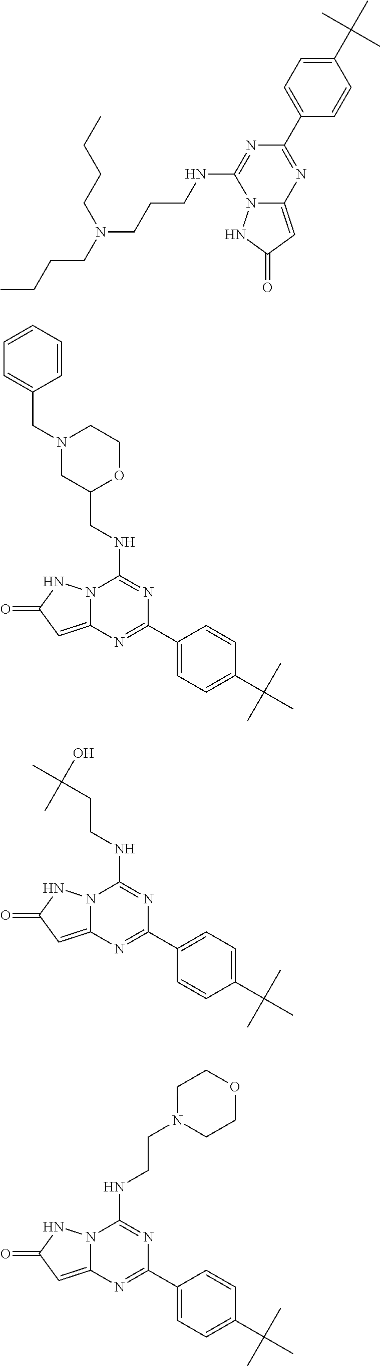

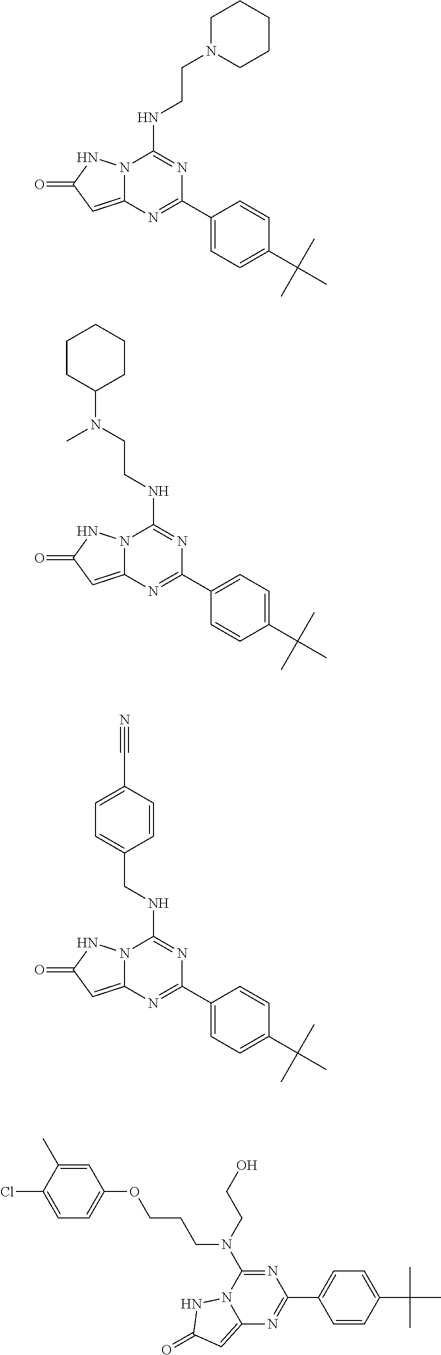 [SMILES: CC(C)(C)c1ccc(-c2nc(NCCN3CCCCC3)n3[nH]c(=O)cc3n2)cc1 ; CN(CCNc1nc(-c2ccc(C(C)(C)C)cc2)nc2cc(=O)[nH]n12)C1CCCCC1 ; CC(C)(C)c1ccc(-c2nc(NCc3ccc(C#N)cc3)n3[nH]c(=O)cc3n2)cc1 ; Cc1cc(OCCCN(CCO)c2nc(-c3ccc(C(C)(C)C)cc3)nc3cc(=O)[nH]n23)ccc1Cl]

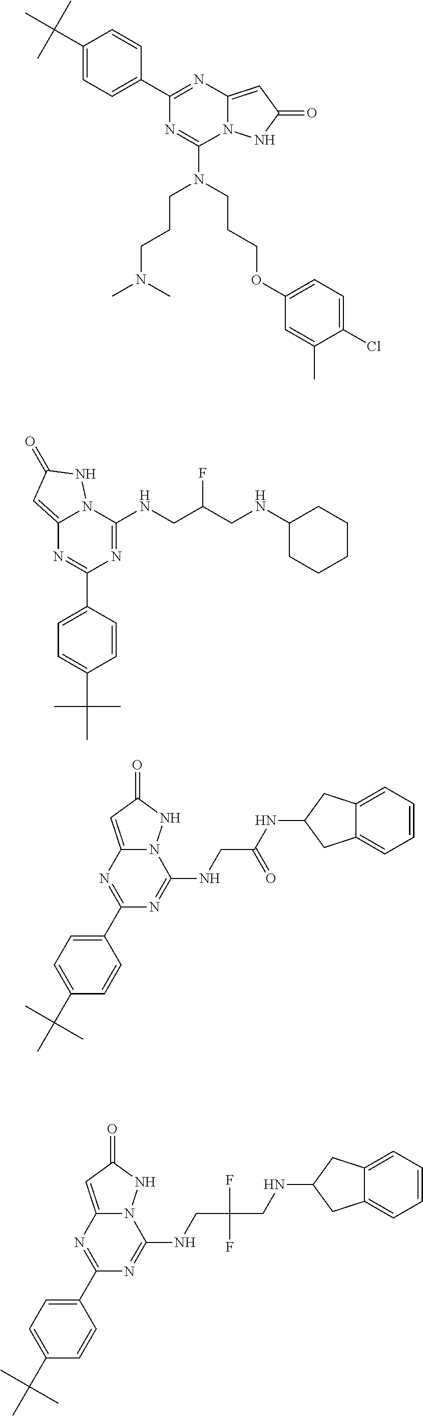 [SMILES: Cc1cc(OCCCN(CCCN(C)C)c2nc(-c3ccc(C(C)(C)C)cc3)nc3cc(=O)[nH]n23)ccc1Cl ; CC(C)(C)c1ccc(-c2nc(NCC(F)CNC3CCCCC3)n3[nH]c(=O)cc3n2)cc1 ; CC(C)(C)c1ccc(-c2nc(NCC(=O)NC3Cc4ccccc4C3)n3[nH]c(=O)cc3n2)cc1 ; CC(C)(C)c1ccc(-c2nc(NCC(F)(F)CNC3Cc4ccccc4C3)n3[nH]c(=O)cc3n2)cc1]

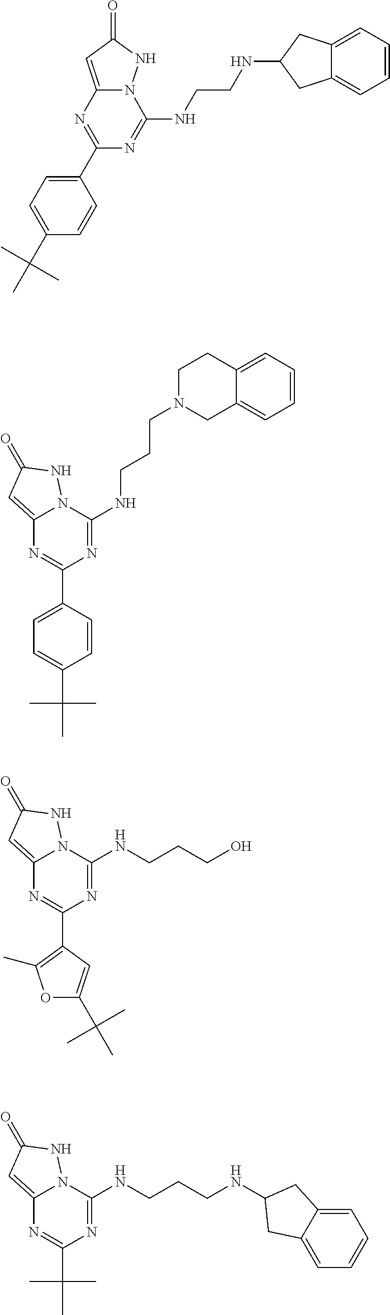 [SMILES: CC(C)(C)c1ccc(-c2nc(NCCNC3Cc4ccccc4C3)n3[nH]c(=O)cc3n2)cc1 ; CC(C)(C)c1ccc(-c2nc(NCCCN3CCc4ccccc4C3)n3[nH]c(=O)cc3n2)cc1 ; Cc1oc(C(C)(C)C)cc1-c1nc(NCCCO)n2[nH]c(=O)cc2n1 ; CC(C)(C)c1nc(NCCCNC2Cc3ccccc3C2)n2[nH]c(=O)cc2n1]

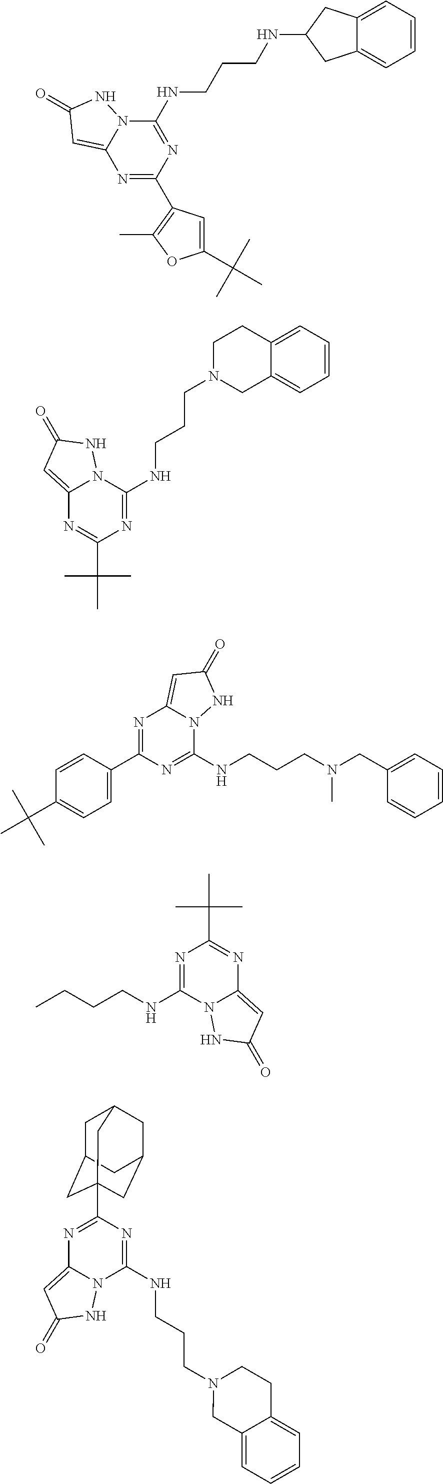

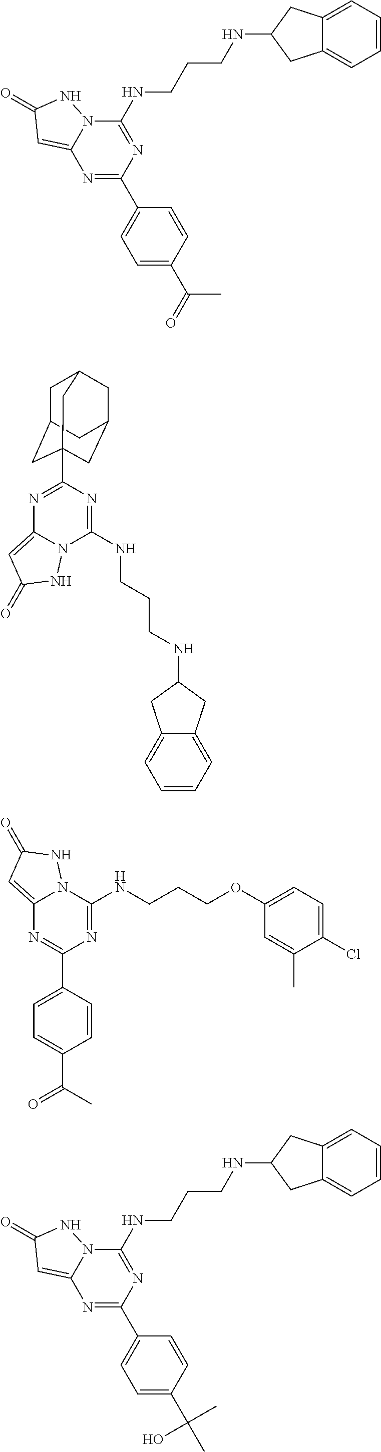 [SMILES: CC(=O)c1ccc(-c2nc(NCCCNC3Cc4ccccc4C3)n3[nH]c(=O)cc3n2)cc1 ; O=c1cc2nc(C34CC5CC(CC(C5)C3)C4)nc(NCCCNC3Cc4ccccc4C3)n2[nH]1 ; CC(=O)c1ccc(-c2nc(NCCCOc3ccc(Cl)c(C)c3)n3[nH]c(=O)cc3n2)cc1 ; CC(C)(O)c1ccc(-c2nc(NCCCNC3Cc4ccccc4C3)n3[nH]c(=O)cc3n2)cc1]

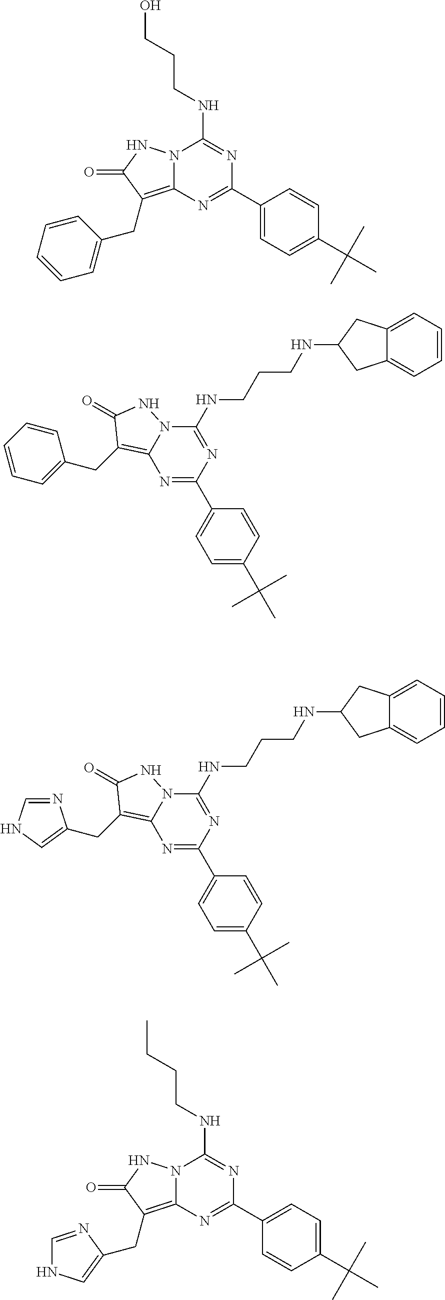

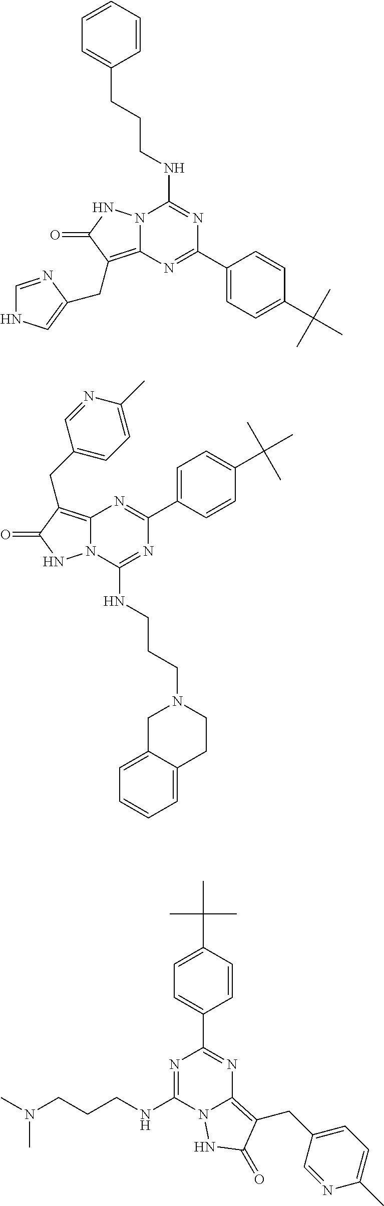

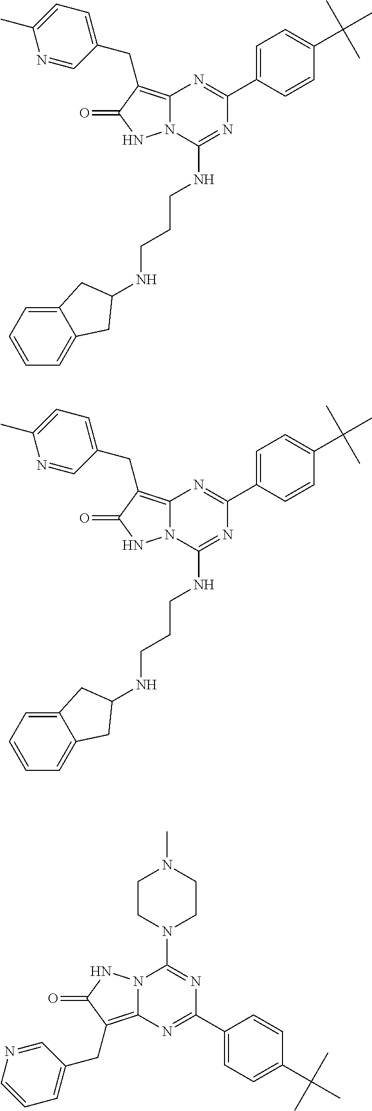

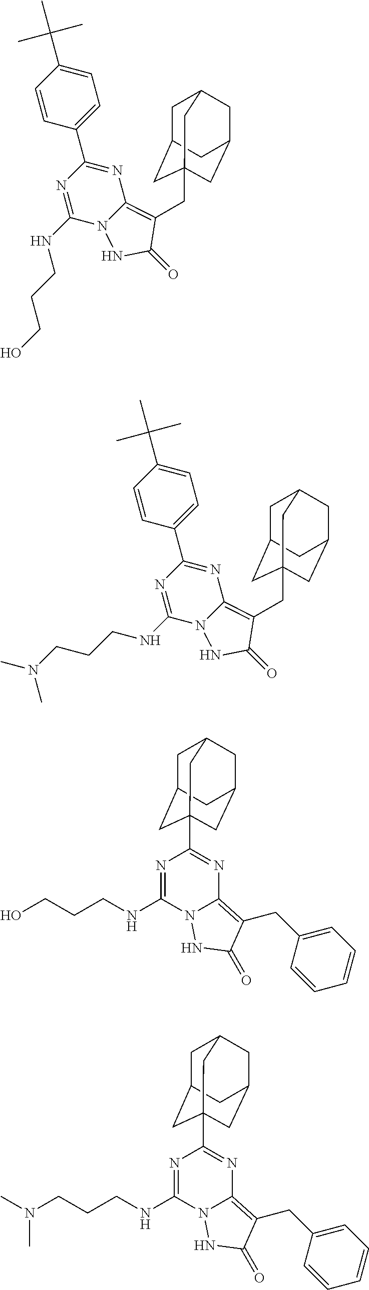 [SMILES: CC(C)(C)c1ccc(-c2nc(NCCCO)n3[nH]c(=O)c(CC45CC6CC(CC(C6)C4)C5)c3n2)cc1 ; CN(C)CCCNc1nc(-c2ccc(C(C)(C)C)cc2)nc2c(CC34CC5CC(CC(C5)C3)C4)c(=O)[nH]n12 ; O=c1[nH]n2c(NCCCO)nc(C34CC5CC(CC(C5)C3)C4)nc2c1Cc1ccccc1 ; CN(C)CCCNc1nc(C23CC4CC(CC(C4)C2)C3)nc2c(Cc3ccccc3)c(=O)[nH]n12]

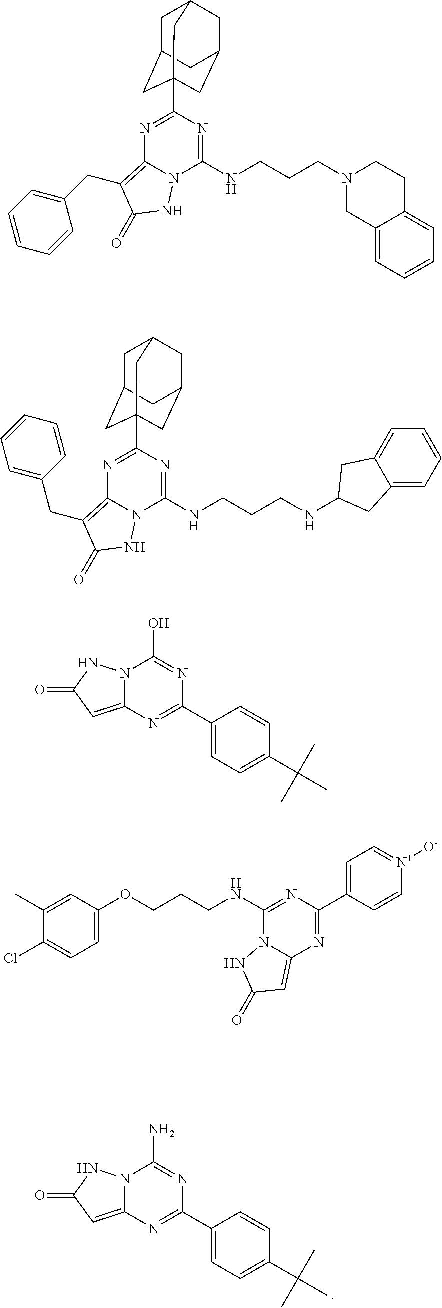

In certain embodiments, the compound used in any of the methods as described herein include, but are not limited to:

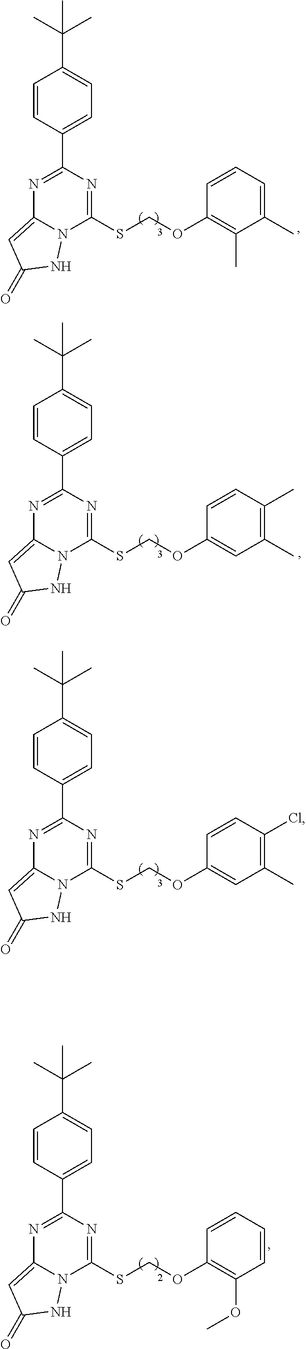

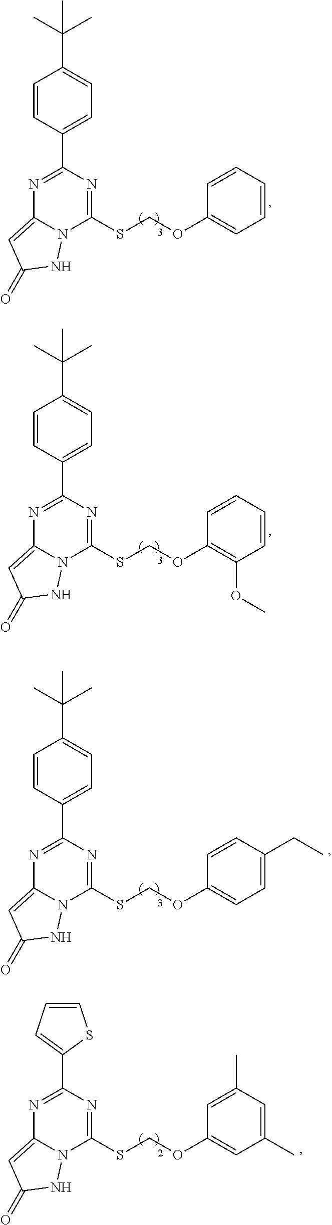

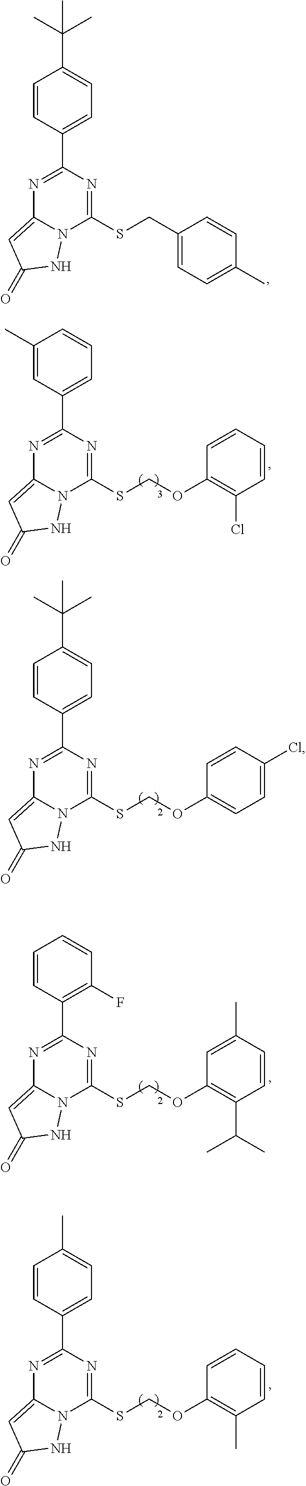

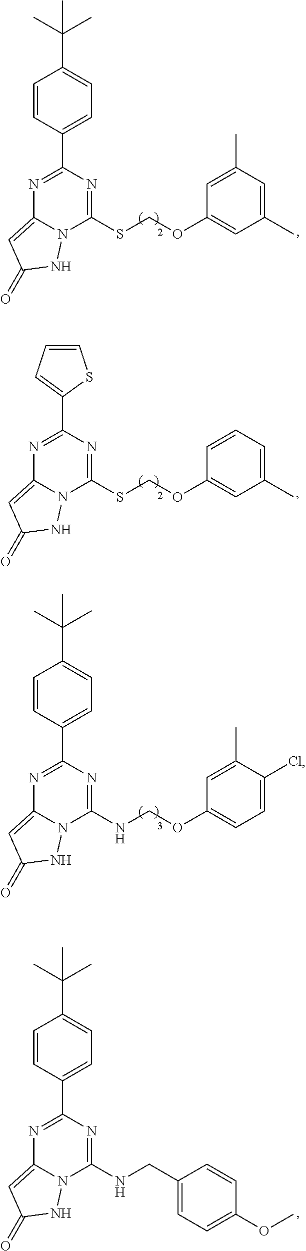

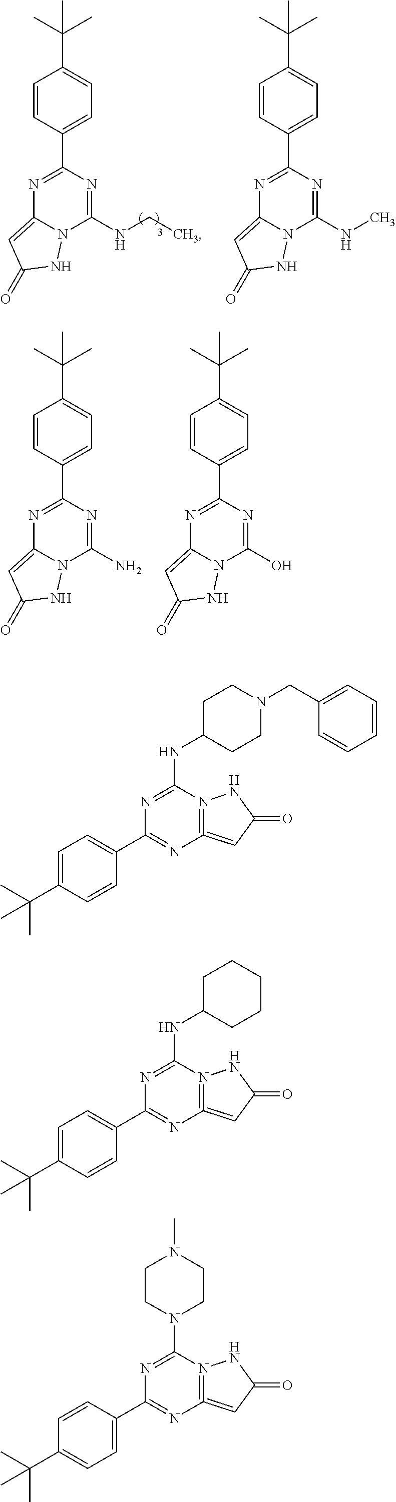

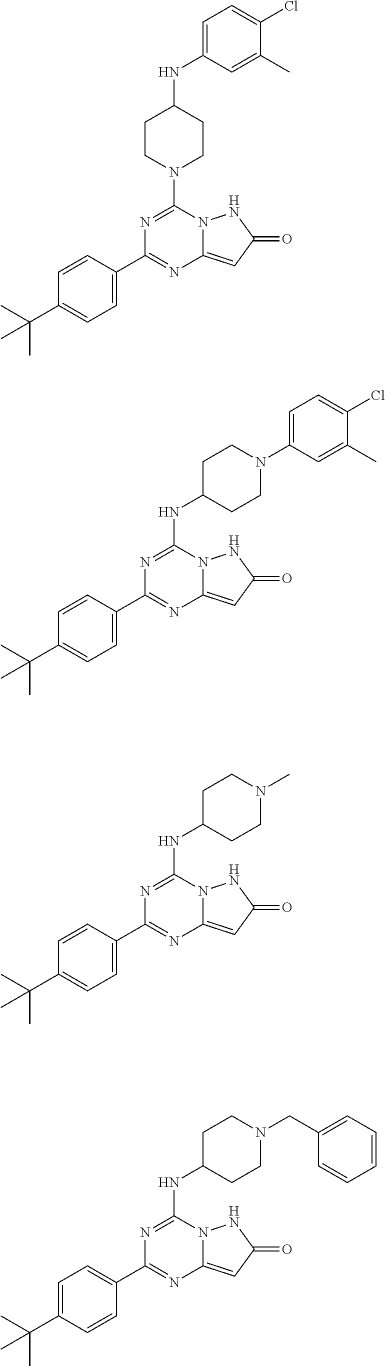

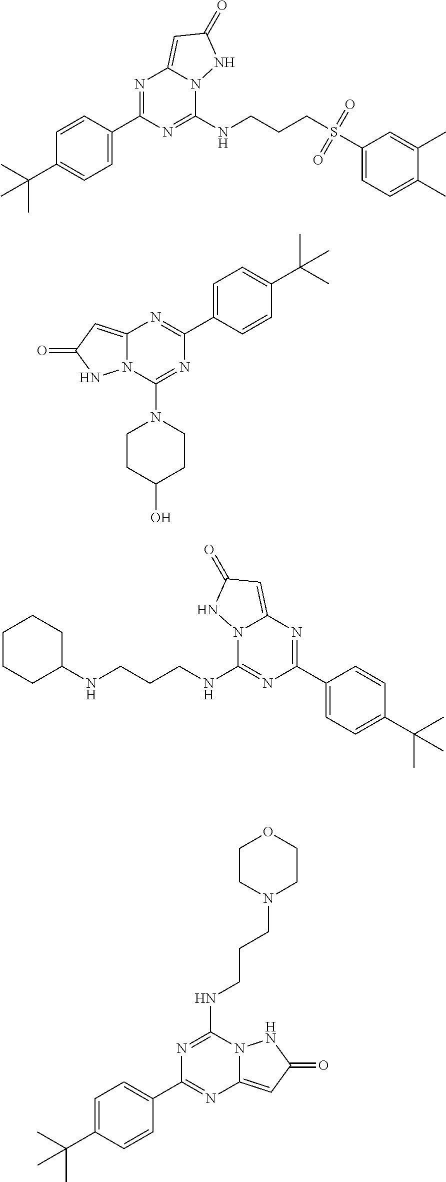

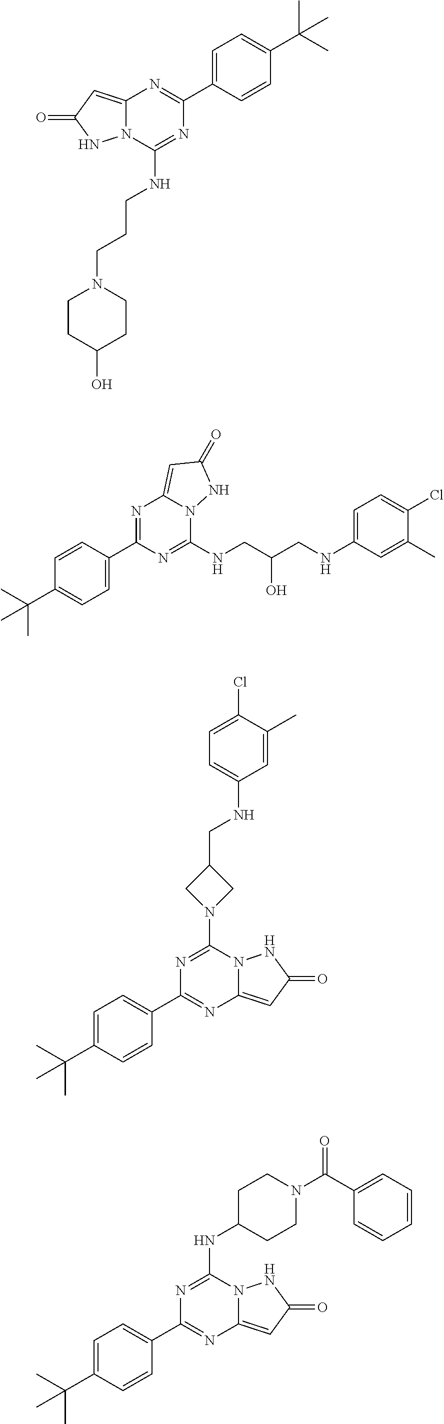

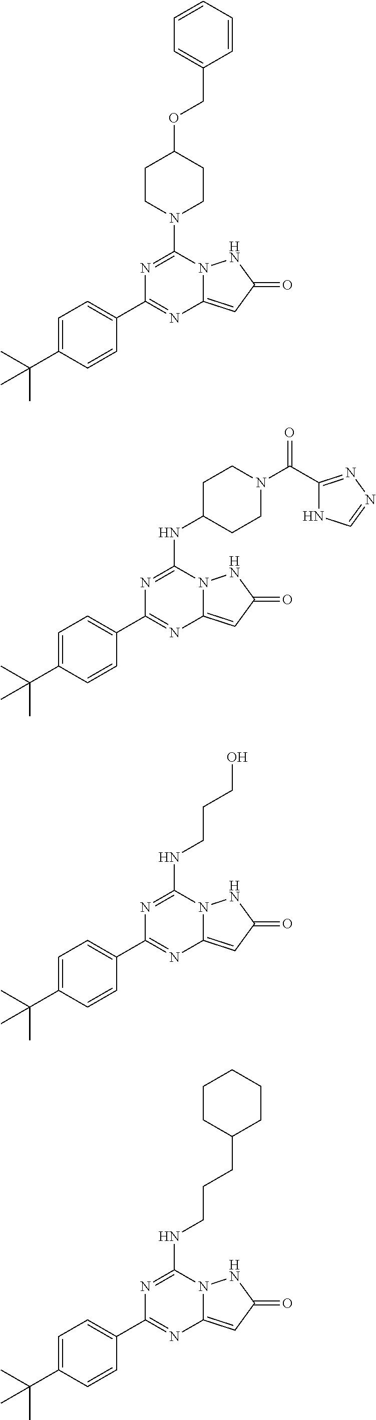 [SMILES: CC(C)(C)c1ccc(-c2nc(N3CCC(OCc4ccccc4)CC3)n3[nH]c(=O)cc3n2)cc1 ; CC(C)(C)c1ccc(-c2nc(NC3CCN(C(=O)c4nnc[nH]4)CC3)n3[nH]c(=O)cc3n2)cc1 ; CC(C)(C)c1ccc(-c2nc(NCCCO)n3[nH]c(=O)cc3n2)cc1 ; CC(C)(C)c1ccc(-c2nc(NCCCC3CCCCC3)n3[nH]c(=O)cc3n2)cc1]

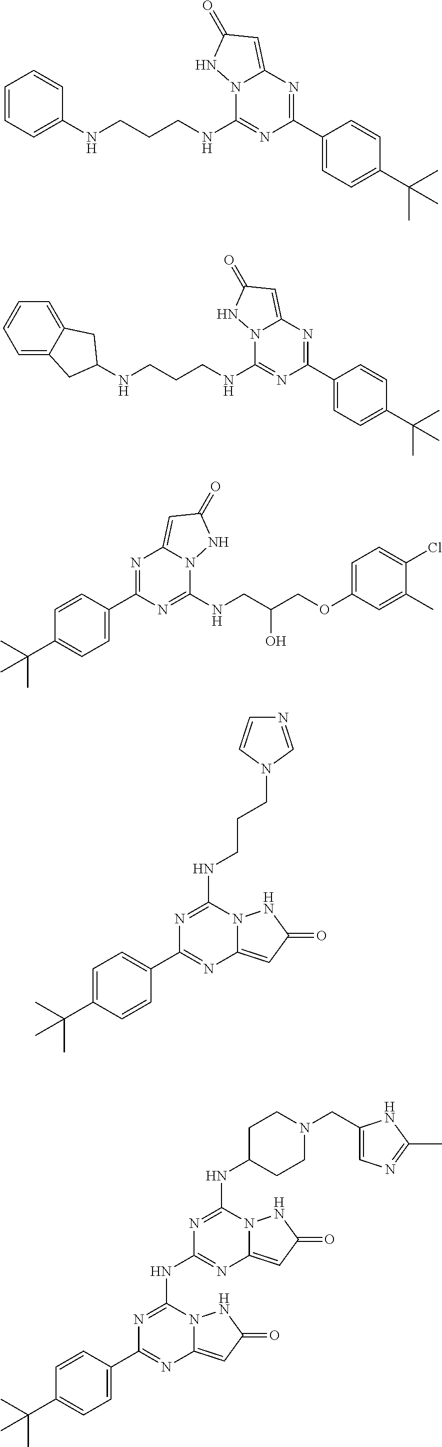

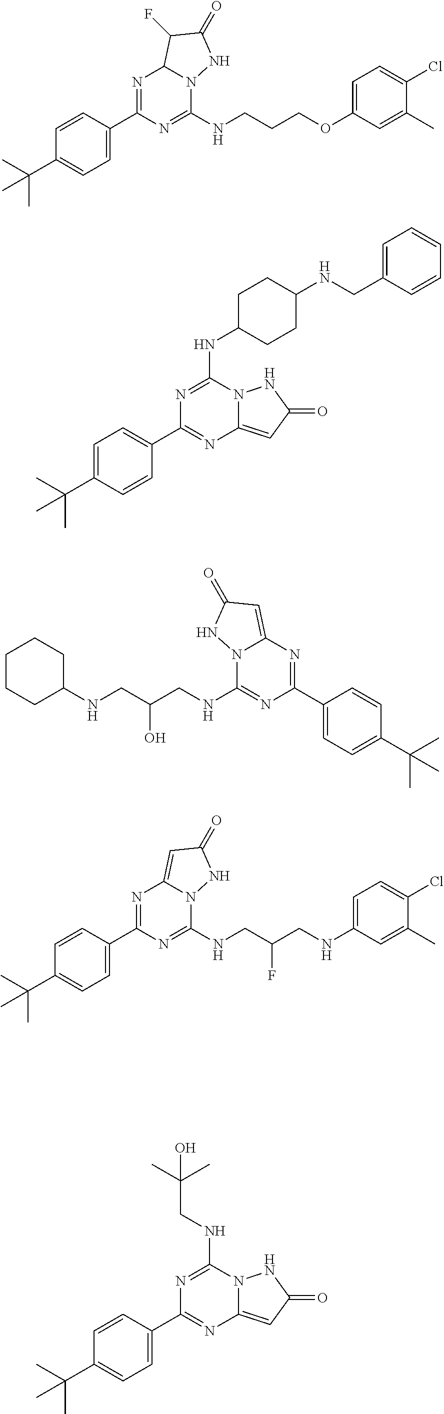

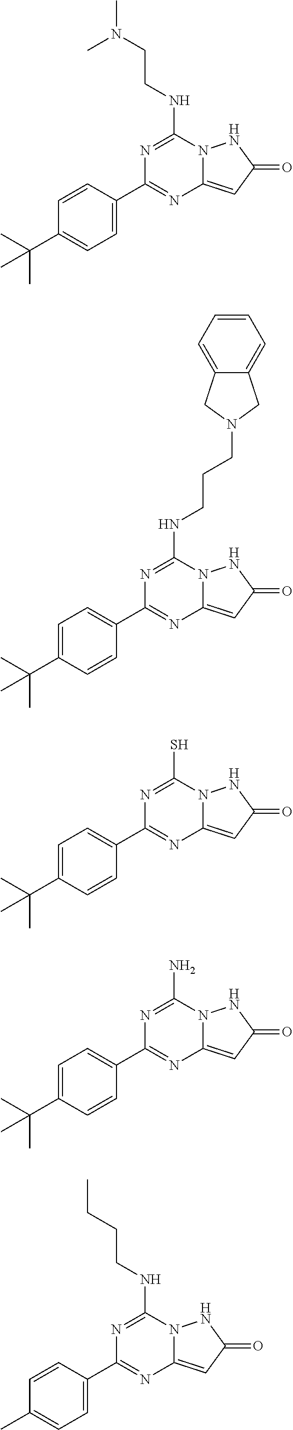 [SMILES: CN(C)CCNc1nc(-c2ccc(C(C)(C)C)cc2)nc2cc(=O)[nH]n12 ; CC(C)(C)c1ccc(-c2nc(NCCCN3Cc4ccccc4C3)n3[nH]c(=O)cc3n2)cc1 ; CC(C)(C)c1ccc(-c2nc(S)n3[nH]c(=O)cc3n2)cc1 ; CC(C)(C)c1ccc(-c2nc(N)n3[nH]c(=O)cc3n2)cc1 ; CCCCNc1nc(-c2ccc(C)cc2)nc2cc(=O)[nH]n12]

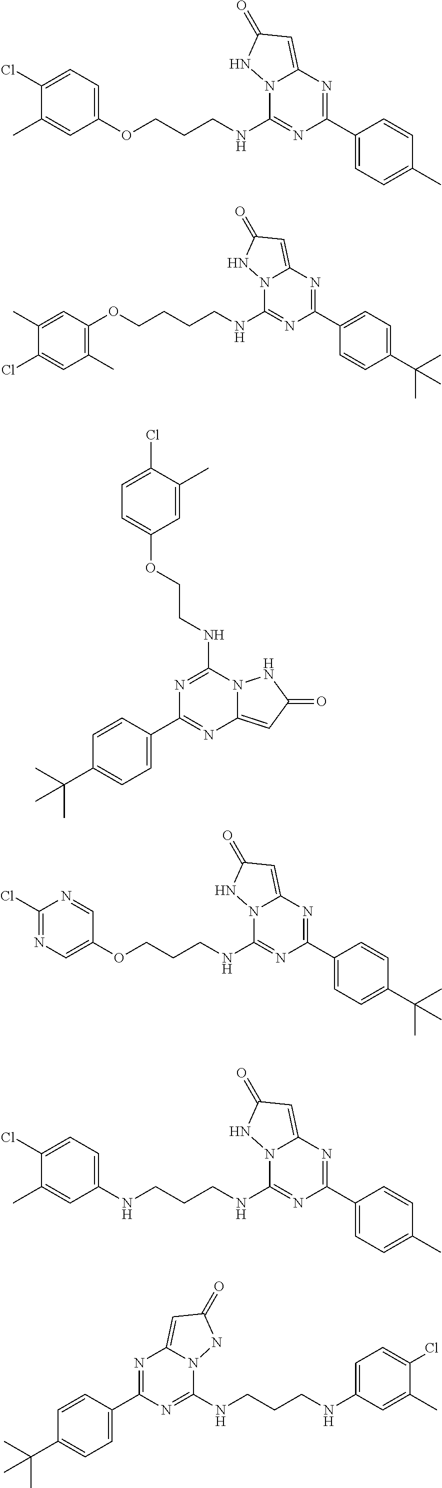

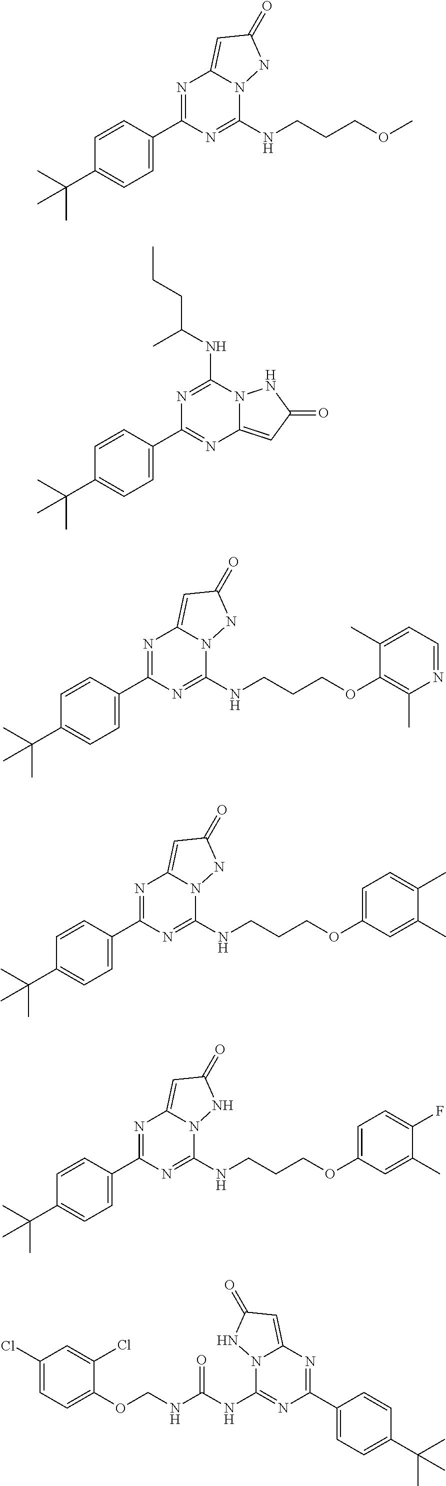

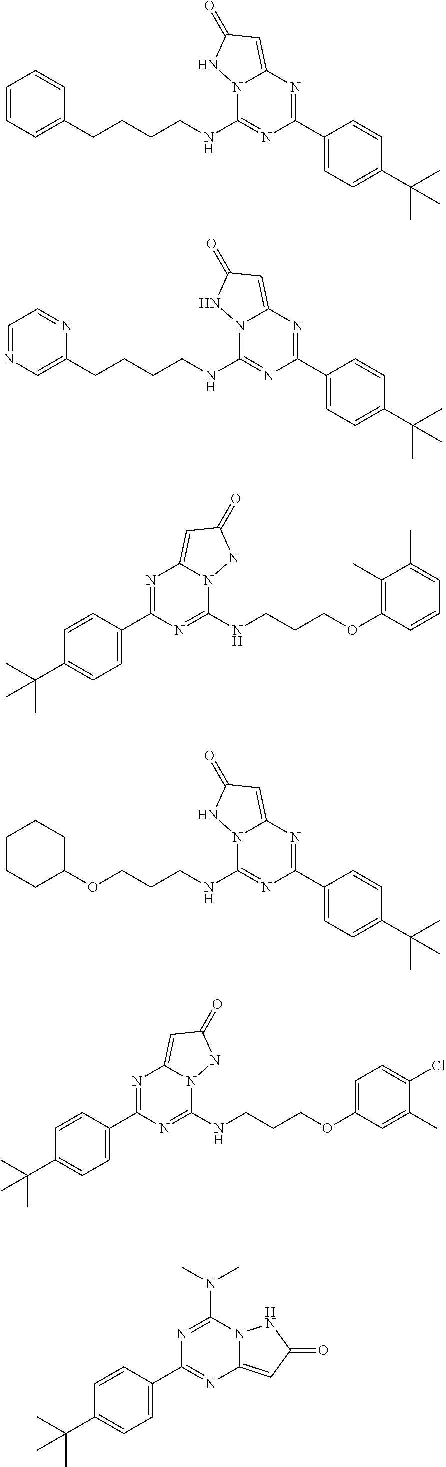

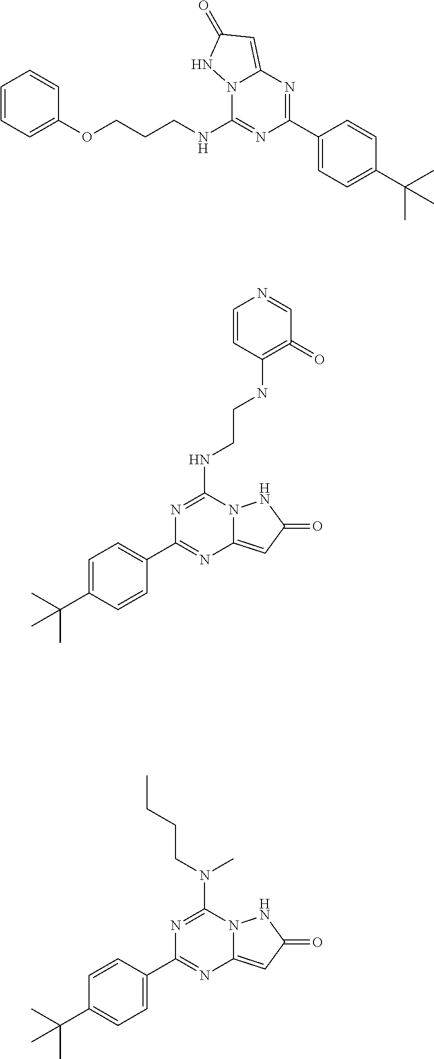

### EXAMPLES

### Chemical Syntheses

In order that the invention described herein may be more fully understood, the following examples are set forth. It should be understood that these examples are for illustrative purposes only and are not to be construed as limiting this invention in any manner.

### LIST OF ABBREVIATIONS

AcOH acetic acid
CV column volumes
d doublet
DCM dichloromethane
DMSO dimethylsulfoxide
Et₂O diethyl ether
EtOAc ethyl acetate
h hours
LCMS liquid chromatography and mass spectrometry
MeCN acetonitrile
MeOH methanol
m multiplet
min minutes
mL millilitre
mol/M mole/molar
mmol millimole
NMR nuclear magnetic resonance
q quartet
quin quintet
rt retention time
s singlet
SNAP KP-SILBiotage trade name for a range of columns
t triplet
TBME tert-butyl methyl ether
TFA 2,2,2-trifluoroacetic acid
TMS trimethylsilyl
μL microliter
μm micrometer

NMR Spectrometers: Bruker Avance III HD 500 MHz NMR; Bruker Avance III HD 250 MHz NMR.

LCMS methods: LCMS Method A refers to high pH analysis using a mobile phase consisting of 2 mM ammonium bicarbonate, buffered to pH10 in a gradient of 1-100% MeCN in water over 2.1 min at a flow rate of 1 mL/min. The stationary phase consisted of a Phenomenex Gemini-NX C18 Part No. 00B-4453-B0, 2.0×50 mm, 3 μm. The experiment was run at 60° C. LCMS Method B refers to low pH analysis using a mobile phase consisting of 0.1% formic acid in a gradient of 5-100% MeCN in water over 5.8 min at a flow rate of 0.6 mL/min. The stationary phase consisted of a Phenomenex Kinetex-XB C18 Part No. 00D-4498-AN, 2.1×100 mm, 1.7 μm. The experiment was run at 40° C. LCMS Method C refers to low pH analysis using a mobile phase consisting of 0.1% formic acid in a gradient of 5-100% MeCN in water over 1.6 min at a flow rate of 1 mL/min. The stationary phase consisted of a Supelco Ascentis Express C18, Part No. 53802-U, 2.1×30 mm, 2.7 μm. The experiment was run at 40° C. LCMS Method D refers to low pH analysis using a mobile phase consisting of 0.1% formic acid in a gradient of 5-100% MeCN in water over 2.7 min at a flow rate of 1 mL/min. The stationary phase consisted of a Waters Atlantis dC18, Part No. 186001295, 2.1×100 mm, 3 μm. The experiment was run at 40° C. LCMS Method E refers to low pH analysis using a mobile phase consisting of 0.1% formic acid in a gradient of 5-100% MeCN in water over 7 min at a flow rate of 0.6 mL/min. The stationary phase consisted of a Waters SymmetryShield RP8, Part No. WAT094257, 2.1×50 mm, 3.5 μm. The experiment was run at room temperature. LCMS Method F refers to low pH analysis using a mobile phase consisting of 0.1% formic acid in a gradient of 5-100% MeCN in water over 5.4 min at a flow rate of 0.6 mL/min. The stationary phase consisted of a Waters Atlantis dC18, Part No. 186001295, 2.1×100 mm, 3 μm. The experiment was run at 40° C. LCMS Method G refers to low pH analysis using a mobile phase consisting of 0.1% formic acid in a gradient of 5-100% MeCN in water over 2.7 min at a flow rate of 1.0 mL/min. The stationary phase consisted of a Waters SymmetryShield RP8, Part No. WAT094257, 2.1×50 mm, 3.5 μm. The experiment was run at 40° C. LCMS Method H refers to low pH analysis for hydrophobic compounds using a mobile phase consisting of 0.1% formic acid in a gradient of 5-100% MeCN in water over 1.83 min, then 100% MeCN for 0.42 min, at a flow rate of 1.2 mL/min. The stationary phase consisted of a Phenomenex Kinetex Core-Shell C8, 2.1×50 mm, 5 μm. The experiment was run at 40° C. HPLC Method A refers to low pH purification using a mobile phase consisting of 0.1% formic acid in a gradient of 30-95% MeCN in water over 10 min at a flow rate of 40 mL/min. The stationary phase consisted of a Waters Sunfire™ C18 OBD™, 30×100 mm, 10 μm. HPLC Method B refers to high pH purification using a mobile phase consisting of 0.2% ammonium hydroxide in a gradient of 30-95% MeCN in water over 10 min at a flow rate of 40 mL/min. The stationary phase consisted of a Waters XBridge™ C18 OBD™, 30×100 mm, 10 μm. HPLC Method C refers to neutral pH purification using a mobile phase consisting of a gradient of 10-100% MeCN in water over 14 min at a flow rate of 40 mL/min. The stationary phase consisted of a Waters Sunfire™ C18 OBD™, 30×100 mm, 10 μm.

Scheme 1 describes the synthetic route to Examples 1 and 2

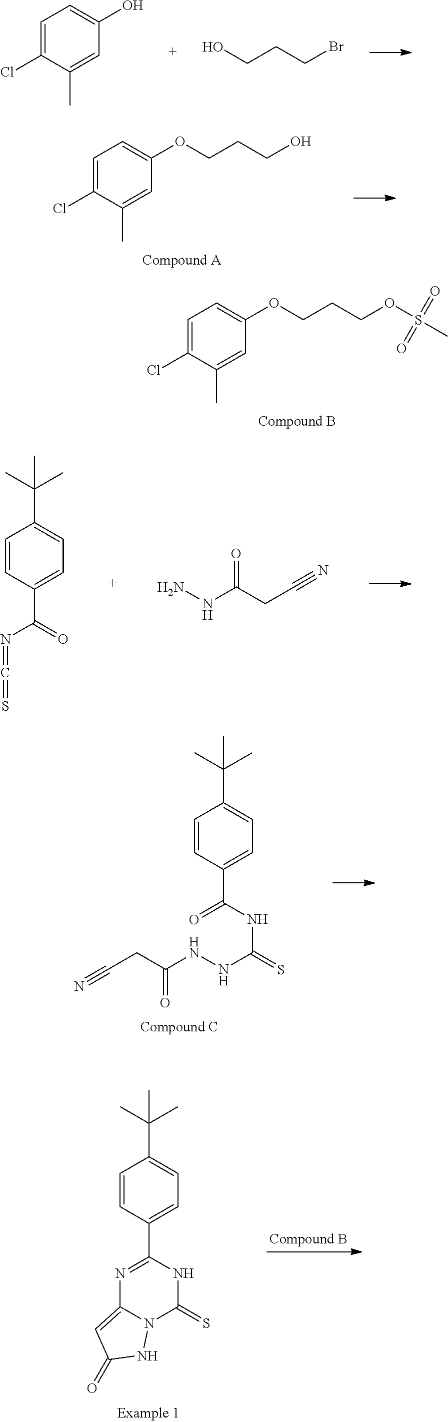

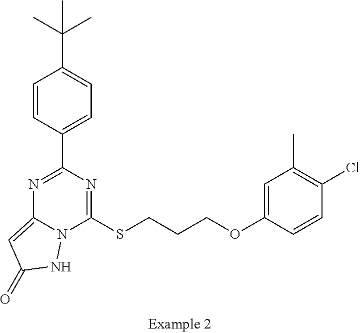 [SMILES: Cc1cc(OCCCSc2nc(-c3ccc(C(C)(C)C)cc3)nc3cc(=O)[nH]n23)ccc1Cl]

### 3-(4-chloro-3-methylphenoxy)propan-1-ol—Compound A

4-chloro-3-methylphenol (1.25 g, 8.77 mmol) was dissolved in 10% NaOH in water (10 mL) and 3-bromopropan-1-ol (637 μl, 7.01 mmol) was added. The reaction mixture was heated to reflux for 18 h. Two further portions of 3-bromopropan-1-ol (637 μL, 7.01 mmol) were added and the reaction was heated to reflux for a further 3 h after each addition. The reaction mixture was diluted with water (10 mL) and extracted with Et₂O (3×20 mL). The combined organics were washed with 10% aq. NaOH (10 mL) and brine (10 mL). The combined organics were dried over Na₂SO₄, filtered and evaporated to dryness to give 1.93 g. The product was purified by column chromatography (Biotage, 50 g SNAP KP-SIL, 100% DCM, 10 CV) to give 1.14 g (73%) of the title compound as a pale yellow oil.

¹H NMR (500 MHz, Chloroform-d) δ 7.21 (d, J=8.7 Hz, 1H), 6.78 (d, J=2.9 Hz, 1H), 6.72-6.62 (m, 1H), 4.08 (t, J=6.0 Hz, 2H), 3.85 (t, J=5.9 Hz, 2H), 2.33 (s, 3H), 2.06-1.98 (m, 2H). LCMS Method A: rt 1.14 min, 100%; m/z 223.1 (MNa⁺)

### 3-(4-chloro-3-methylphenoxy)propyl methanesulfonate—Compound B

3-(4-chloro-3-methylphenoxy)propan-1-ol (300 mg, 1.5 mmol) was dissolved in DCM (3 mL) and cooled to 0° C. Triethylamine (414 μL, 2.99 mmol) was added followed by dropwise addition of methanesulfonyl chloride (127 μL, 1.64 mmol) in DCM (3 mL). The reaction mixture was stirred at 0° C. for 3 h. The reaction mixture was diluted with DCM (30 mL), poured onto ice water and the aqueous layer was removed. The organics were washed with 2×10% HCl (10 mL), brine (5 mL), sat NaHCO₃ (10 mL) and brine (10 mL). The organics were filtered through a hydrophobic frit and evaporated to dryness to give 375 mg of the title compound as a pale yellow oil.

¹H NMR (500 MHz, Chloroform-d) δ 7.24-7.16 (m, 1H), 6.80-6.74 (m, 1H), 6.70-6.60 (m, 1H), 4.43 (t, J=6.1 Hz, 2H), 4.05 (t, J=5.9 Hz, 2H), 2.99 (s, 3H), 2.34 (s, 3H), 2.25-2.16 (m, 2H). LCMS Method A: rt 1.59 min, 100%; m/z 296.0 (MNH₄⁺).

### N-({[(4-tert-butylphenyl)formamido]methanethioyl}amino)-2-cyanoacetamide—Compound C

2-cyanoacetohydrazide (1.64 g, 16.51 mmol) was dissolved in acetone (40 mL) and 4-tert-butylbenzoyl isothiocyanate (3.62 g, 16.51 mmol) was added portionwise. The reaction mixture was heated to reflux for 90 min. The reaction mixture was evaporated to dryness to give an orange oil. This was triturated with water. The solid formed was filtered off and dried under vacuum, affording 5.06 g of the title compound.

¹H NMR (500 MHz, DMSO-d6) δ 12.58 (s, 1H), 11.66 (s, 1H), 11.22 (s, 1H), 7.93 (d, J=8.4 Hz, 2H), 7.56 (d, J=8.4 Hz, 2H), 3.88 (s, 2H), 1.32 (s, 9H). LCMS Method A: rt 1.07 min, 100%; m/z 319.0 (MH⁺).

### 2-(4-tert-butylphenyl)-4-sulfanylidene-3H,4H,6H,7H-pyrazolo[1,5-a][1,3,5]triazin-7-one—Example 1

N-({[(4-tert-butylphenyl)formamido]methanethioyl}amino)-2-cyanoacetamide (6.06 g, 19.03 mmol) was dissolved in 5% KOH in water (60 mL) and the reaction mixture was heated to reflux for 90 min. The reaction mixture was acidified to pH 1 with 2M aq. HCl. The precipitate formed was collected and dried under vacuum. The product was then triturated with heptanes, affording 5.13 g of the title compound.

¹H NMR (500 MHz, DMSO-d6) δ 13.54 (s, 1H), 11.77 (s, 1H), 8.07 (d, J=8.5 Hz, 2H), 7.57 (d, J=8.4 Hz, 2H), 6.02 (s, 1H), 1.33 (s, 9H). LCMS Method A: rt 0.84 min, 83%; m/z 301.0 (MH⁺).

### 2-(4-tert-butylphenyl)-4-{[3-(4-chloro-3-methylphenoxy)propyl]sulfanyl}-6H,7H-pyrazolo[1,5-a][1,3,5]triazin-7-one—Example 2

2-(4-tert-butylphenyl)-4-sulfanylidene-3H,4H,6H,7H-pyrazolo[1,5-a][1,3,5]triazin-7-one (340 mg, 1.13 mmol) and K₂CO₃ (313 mg, 2.26 mmol) were dissolved in acetone (6 mL) and 3-(4-chloro-3-methylphenoxy)propyl methanesulfonate (375 mg, 1.35 mmol) was added. The reaction mixture was heated at 65° C. for 18 h. The reaction mixture was evaporated to dryness. The crude product was diluted with DCM (60 mL) and water (20 mL) and a pale orange suspension formed. EtOAc (20 mL) was added which gave solution. The aqueous layer was removed and the organics were washed with water (3×15 mL). The organic layer was passed through a hydrophobic frit and evaporated to dryness. The product was purified by column chromatography (Biotage, 25 g SNAP KP-SIL, 0-50% EtOAc in Heptane, 10 CV) to give 36 mg of the title compound as a pale yellow solid.

¹H NMR (500 MHz, Chloroform-d) δ 10.37 (s, 1H), 8.45-8.33 (m, 2H), 7.57-7.44 (m, 2H), 7.22 (d, J=8.7 Hz, 1H), 6.82 (d, J=2.9 Hz, 1H), 6.77-6.66 (m, 1H), 6.03 (s, 1H), 4.14 (t, J=5.7 Hz, 2H), 3.68 (t, J=7.2 Hz, 2H), 2.48-2.37 (m, 2H), 2.33 (s, 3H), 1.37 (s, 9H). LCMS Method B: rt 5.20 min, 97%; m/z 483.1 (MH⁺)

Examples 3-13 can be synthesized in a manner analogous to that for Example 2 using the appropriate isothiocyanates and alkylating agents. Scheme 2 describes the synthetic route to Example 14.

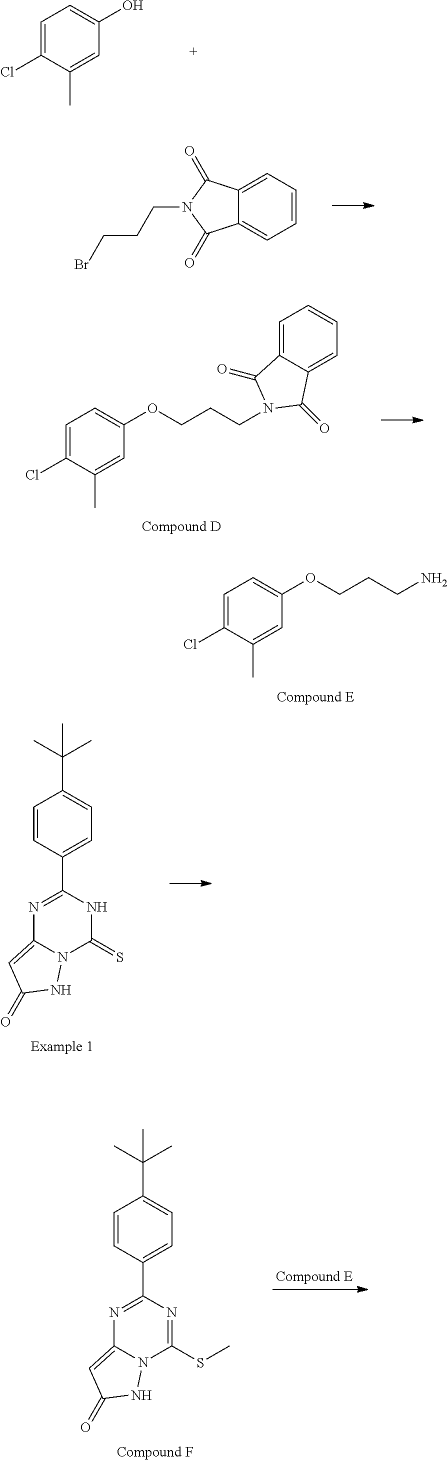

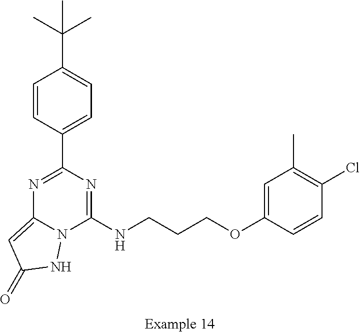 [SMILES: Cc1cc(OCCCNc2nc(-c3ccc(C(C)(C)C)cc3)nc3cc(=O)[nH]n23)ccc1Cl]

### 2-[3-(4-chloro-3-methylphenoxy)propyl]-2,3-dihydro-1H-isoindole-1,3-dione—Compound D

4-chloro-3-methylphenol (1 g, 7.01 mmol) was dissolved in THF (10 mL) at room temperature. Tetrabutyl ammonium iodide (518.11 mg, 1.4 mmol) and 2-(3-bromopropyl)-1H-isoindole-1,3(2H)-dione (2.07 g, 7.71 mmol) were added followed by Cs₂CO₃ (4.11 g, 12.62 mmol). The reaction mixture was stirred at 50° C. for 18 h, then at room temperature for 72 h. A precipitate was filtered off and triturated from MeOH to give 1.15 g of the title compound as a white powder.

¹H NMR (500 MHz, DMSO-d6) 7.91-7.78 (m, 4H), 7.23 (d, J=8.7 Hz, 1H), 6.74 (d, J=2.9 Hz, 1H), 6.69-6.59 (m, 1H), 4.00 (t, J=5.8 Hz, 2H), 3.76 (t, J=6.7 Hz, 2H), 2.23 (s, 3H), 2.10-2.00 (m, 2H). LCMS Method A: rt 1.77 min, 98%; m/z 347.0 (M+NH₃, 100%), 330 (MH⁺).

### 4-(3-aminopropoxy)-1-chloro-2-methylbenzene—Compound E

2-[3-(4-chloro-3-methylphenoxy)propyl]-2,3-dihydro-1H-isoindole-1,3-dione (1.15 g, 3.49 mmol) was dissolved in Ethanol (2 mL). Hydrazine hydrate (1:1) (1.02 mL, 20.92 mmol) was added and the reaction was heated to 60° C. for 3 h. At 1 h the reaction mixture was immobile due to a white ppt, and further ethanol (4 mL) was added to aid stirring. The reaction mixture was cooled to room temperature and evaporated to dryness. The resulting solid was washed with Et₂O and filtered, the filtrate was evaporated to dryness to give the title compound as an off-white solid (638 mg, 92%).

¹H NMR (500 MHz, DMSO-d6) δ 8.08 (br. s, 2H), 7.30 (d, J=8.8 Hz, 1H), 6.96 (d, J=2.9 Hz, 1H), 6.81 (dd, J=8.7, 3.0 Hz, 1H), 4.05 (t, J=6.2 Hz, 2H), 2.97-2.86 (m, 2H), 2.03 (quin, J=6.4 Hz, 2H). LCMS Method A: rt 1.44 min, 99%; m/z 200.0 (MH⁺)

### 2-(4-tert-butylphenyl)-4-(methylsulfanyl)-6H,7H-pyrazolo[1,5-a][1,3,5]triazin-7-one—Compound F

2-(4-tert-butylphenyl)-4-sulfanylidene-3H,4H,6H,7H-pyrazolo[1,5-a][1,3,5]triazin-7-one (1 g, 3.3 mmol) and K₂CO₃ (920 mg, 6.66 mmol) were stirred in acetone (20 mL) and heated to 60° C. Iodomethane (228 μl, 3.7 mmol) in acetone (10 mL) was added and the reaction mixture heated to reflux for 1 h. The reaction was concentrated and dry-loaded onto silica. Column chromatography (Biotage, 100 g SNAP KP-SIL, 0-5% MeOH in DCM 10 CV) afforded the title compound as a yellow solid (1 g, 48%).

¹H NMR (500 MHz, DMSO-d6) δ 11.72 (s, 1H), 8.42-8.29 (m, 2H), 7.63-7.47 (m, 2H), 5.95 (s, 1H), 2.79 (s, 3H), 1.34 (s, 9H). LCMS Method A: rt 1.17 min, 85%; m/z 315.0 (MH⁺).

### 2-(4-tert-butylphenyl)-4-{[3-(4-chloro-3-methylphenoxy)propyl]amino}-6H,7H-pyrazolo[1,5-a][1,3,5]triazin-7-one—Example 14

2-(4-tert-butylphenyl)-4-(methylsulfanyl)-6H,7H-pyrazolo[1,5-a][1,3,5]triazin-7-one (50 mg, 0.16 mmol) was dissolved in acetonitrile (1 mL). 4-(3-aminopropoxy)-1-chloro-2-methylbenzene (95 mg, 0.48 mmol) was added. 250 μL DMF was added to aid solubilisation. The reaction mixture was heated to 130° C. in the microwave for 3 h. The reaction mixture was evaporated to dryness and purified by column chromatography (Biotage, 10 g SNAP KP-SIL, 0-100% TBME in heptane, 12 CV), affording the title compound as a yellow solid (5.1 mg, 7%).

¹H NMR (250 MHz, DMSO-d6) δ 8.41-8.29 (m, 1H), 8.25-8.15 (m, 2H), 7.47-7.37 (m, 2H), 7.29-7.17 (m, 1H), 6.98-6.85 (m, 1H), 6.82-6.73 (m, 1H), 5.69 (s, 1H), 4.08 (t, J=5.9 Hz, 2H), 3.85-3.70 (m, 2H), 2.23 (s, 3H), 2.18-2.05 (m, 2H), 1.31 (s, 9H). LCMS Method B: rt 4.83 min, 97%; m/z 466.1 (MH⁺).

Examples 15 to 36 were synthesized by reacting Compound F with commercially available amines as illustrated in Scheme 3.

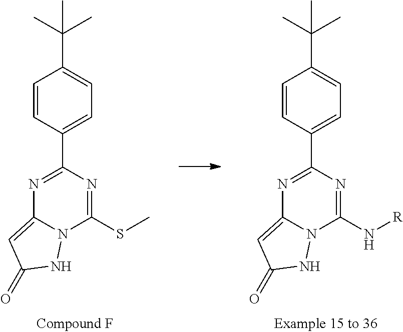

### 2-(4-tert-butylphenyl)-4-{[(4-methoxyphenyl)methyl]amino}-6H,7H-pyrazolo[1,5-a][1,3,5]triazin-7-one—Example 15

2-(4-tert-butylphenyl)-4-(methylsulfanyl)-6H,7H-pyrazolo[1,5-a][1,3,5]triazin-7-one (100 mg, 0.32 mmol) was dissolved in acetonitrile (2 mL). p-methoxy benzylamine (131 mg, 0.95 mmol) was added. The reaction mixture was heated to 130° C. in a microwave for 1 h. The reaction mixture was evaporated to dryness and purified by column chromatography (Biotage, 10 g SNAP KP-SIL, 0-100% TBME in heptane, 10 CV, then 0-10% MeOH in TBME, 5 CV), affording the title compound as a white solid (67 mg, 50%).

¹H NMR (500 MHz, Methanol-d4) δ 8.33-8.21 (m, 2H), 7.53-7.47 (m, 2H), 7.39-7.34 (m, 2H), 6.89-6.84 (m, 2H), 5.66 (s, 1H), 4.79 (s, 2H), 3.73 (s, 3H), 1.36 (s, 9H). LCMS Method B: rt 4.20 min, 97%; m/z 404.2 (MH⁺).

### 4-(butylamino)-2-(4-tert-butylphenyl)-6H,7H-pyrazolo[1,5-a][1,3,5]triazin-7-one—Example 16

2-(4-tert-butylphenyl)-4-(methylsulfanyl)-6H,7H-pyrazolo[1,5-a][1,3,5]triazin-7-one (50 mg, 0.16 mmol) was dissolved in butan-1-amine (500 μL, 5.06 mmol). The reaction mixture was heated to 115° C. in a microwave for 15 min. The reaction mixture was evaporated to dryness and purified by column chromatography (Biotage, 10 g SNAP KP-SIL, 25-100% EtOAc in Heptane 10 CV), affording the title compound as a white solid (15 mg, 28%).

¹H NMR (500 MHz, Methanol-d4) δ 8.26 (d, J=8.6 Hz, 2H), 7.50 (d, J=8.6 Hz, 2H), 5.63 (s, 1H), 3.72 (t, J=7.1 Hz, 2H), 1.80-1.69 (m, 2H), 1.55-1.43 (m, 2H), 1.36 (s, 9H), 1.02 (t, J=7.4 Hz, 3H). LCMS Method B: rt 4.38 min, 96%; m/z 340.2 (MH⁺).

### 2-(4-tert-butylphenyl)-4-(methylamino)-6H,7H-pyrazolo[1,5-a][1,3,5]triazin-7-one—Example 17

2-(4-tert-butylphenyl)-4-(methylsulfanyl)-6H,7H-pyrazolo[1,5-a][1,3,5]triazin-7-one (50 mg, 0.16 mmol) was dissolved in methylamine (297 μL, 2.39 mmol, 33% wt in ethanol). The reaction mixture was heated to 115° C. in a microwave for 15 mins. The reaction mixture was evaporated to dryness and purified by column chromatography (Biotage, 10 g SNAP KP-SIL, 0-100% TBME in Heptane 10 CV, 100% TBME 5 CV), affording the title compound as a white solid (19 mg, 37%).

¹H NMR (500 MHz, Methanol-d4) δ 8.32-8.24 (m, 2H), 7.57-7.45 (m, 2H), 5.66 (s, 1H), 3.23 (s, 3H), 1.37 (s, 9H). LCMS Method B: rt 3.52 min, 92%; m/z 298.2 (MH⁺)

### 2-(4-tert-butylphenyl)-4-(4-methylpiperazin-1-yl)-6H,7H-pyrazolo[1,5-a][1,3,5]triazin-7-one—Example 18

2-(4-tert-butylphenyl)-4-(methylsulfanyl)-6H,7H-pyrazolo[1,5-a][1,3,5]triazin-7-one (100 mg, 0.32 mmol) was dissolved in 1-methylpiperazine (1 mL, 9.02 mmol). The reaction was stirred at room temperature for 2 h, then concentrated. The residue was dissolved in DMSO and purified using HPLC Method A, affording the title compound as a yellow powder (11.5 mg, 9%).

¹H NMR (500 MHz, Chloroform-d) δ 8.29 (s, 1H), 8.19 (d, J=8.4 Hz, 2H), 7.40 (d, J=8.5 Hz, 2H), 5.77 (s, 1H), 4.58 (s, 4H), 3.00 (s, 4H), 2.52 (s, 3H), 1.29 (s, 9H). LCMS Method B: rt 2.28 min, 93%; m/z 367.3 (MH⁺)

### 2-(4-tert-butylphenyl)-4-[(1-methylpiperidin-4-yl)amino]-6H,7H-pyrazolo[1,5-a][1,3,5]triazin-7-one—Example 19

2-(4-tert-butylphenyl)-4-(methylsulfanyl)-6H,7H-pyrazolo[1,5-a][1,3,5]triazin-7-one (80 mg, 0.25 mmol) and 1-methylpiperidin-4-amine (32 μl, 0.25 mmol) were dissolved in 1,4-Dioxane (1 mL). The reaction mixture was heated to 135° C. in microwave for 3 h, then concentrated and purified using HPLC Method A, affording the title compound (32.1 mg, 33%).

¹H NMR (500 MHz, Chloroform-d) δ 8.51 (s, 1H), 8.24 (d, J=8.4 Hz, 2H), 7.44 (d, J=8.5 Hz, 2H), 7.20 (d, J=6.9 Hz, 1H), 5.80 (s, 1H), 4.46 (s, 1H), 3.52 (m, 2H), 2.87 (d, J=12.1 Hz, 2H), 2.80 (s, 3H), 2.43-2.28 (m, 2H), 2.23 (d, J=11.9 Hz, 2H), 1.33 (s, 9H). LCMS Method B: rt 2.21 min, 100%; m/z 381.3 (MH⁺)

### 2-(4-tert-butylphenyl)-4-(4-hydroxypiperidin-1-yl)-6H,7H-pyrazolo[1,5-a][1,3,5]triazin-7-one—Example 20

2-(4-tert-butylphenyl)-4-(methylsulfanyl)-6H,7H-pyrazolo[1,5-a][1,3,5]triazin-7-one (80 mg, 0.25 mmol) and piperidin-4-ol (77 mg, 0.76 mmol) were dissolved in Pyridine (0.5 mL). The reaction was heated to 50° C. in a sealed tube for 18 h, then concentrated and purified using HPLC Method A, affording the title compound (24 mg, 24%).

¹H NMR (500 MHz, Methanol-d4) δ 8.22 (d, J=8.7 Hz, 2H), 7.49 (d, J=8.7 Hz, 2H), 5.67 (s, 1H), 4.96 (d, J=13.3 Hz, 2H), 4.05-3.90 (m, 1H), 3.89-3.76 (m, 2H), 2.09-1.99 (m, 2H), 1.76-1.57 (m, 2H), 1.36 (s, 9H). LCMS Method B: rt 3.32 min, 99%; m/z 368.3 (MH⁺)

### 4-[4-(benzyloxy)piperidin-1-yl]-2-(4-tert-butylphenyl)-6H,7H-pyrazolo[1,5-a][1,3,5]triazin-7-one—Example 21

2-(4-tert-butylphenyl)-4-(methylsulfanyl)-6H,7H-pyrazolo[1,5-a][1,3,5]triazin-7-one (60 mg, 0.19 mmol) and 4-(benzyloxy)piperidine (62 mg, 0.32 mmol) were dissolved in Pyridine (0.5 mL). The reaction mixture was heated to 120° C. in microwave for 16 h, then concentrated and purified using HPLC Method A, affording the title compound (17.8 mg, 20%).

¹H NMR (500 MHz, Chloroform-d) δ 8.29 (d, J=8.7 Hz, 2H), 7.47 (d, J=8.7 Hz, 2H), 7.41-7.33 (m, 4H), 7.32-7.27 (m, 1H), 5.83 (s, 1H), 4.61 (s, 2H), 4.56-4.47 (m, 2H), 4.06-3.98 (m, 2H), 3.81-3.72 (m, 1H), 2.10-2.01 (m, 2H), 1.93-1.82 (m, 2H), 1.35 (s, 9H). LCMS Method B: rt 4.82 min, 100%; m/z 458.3 (MH⁺)

### 2-(4-tert-butylphenyl)-4-[(3-hydroxypropyl)amino]-6H,7H-pyrazolo[1,5-a][1,3,5]triazin-7-one—Example 22

2-(4-tert-butylphenyl)-4-(methylsulfanyl)-6H,7H-pyrazolo[1,5-a][1,3,5]triazin-7-one (80 mg, 0.25 mmol) and 3-aminopropan-1-ol (537 mg, 7.15 mmol) were stirred in a sealed tube at room temperature for 22 h. The mixture was then concentrated and purified using HPLC Method A, affording the title compound (52.0 mg, 32%).

¹H NMR (500 MHz, Methanol-d4) δ 8.34-8.14 (m, 2H), 7.58-7.40 (m, 2H), 5.66 (s, 1H), 3.82 (t, J=6.8 Hz, 2H), 3.72 (t, J=6.1 Hz, 2H), 1.98 (quin, J=6.6 Hz, 2H), 1.36 (s, 9H). LCMS Method A: rt 1.71 min, 99%; m/z 342.5 (MH⁺)

### 2-(4-tert-butylphenyl)-4-[(3-phenylpropyl)amino]-6H,7H-pyrazolo[1,5-a][1,3,5]triazin-7-one—Example 23

2-(4-tert-butylphenyl)-4-(methylsulfanyl)-6H,7H-pyrazolo[1,5-a][1,3,5]triazin-7-one (100 mg, 0.32 mmol) and 3-phenylpropan-1-amine (51 mg, 0.64 mmol) were dissolved in Pyridine (1.0 mL). The reaction was heated to 80° C. in a sealed tube for 3 h, then concentrated and filtered through a pad of silica gel. The filtrate was concentrated and triturated with 1:1 toluene:heptane to afford the title compound as a white solid (5 mg, 4%).

¹H NMR (500 MHz, Methanol-d4) δ 8.19 (d, J=8.5 Hz, 2H), 7.49 (d, J=8.5 Hz, 2H), 7.37-7.04 (m, 5H), 5.63 (s, 1H), 3.72 (t, J=7.2 Hz, 2H), 2.77 (t, J=7.4 Hz, 2H), 2.09 (quin, J=7.3 Hz, 2H), 1.37 (s, 9H). LCMS Method B: rt 4.59 min, 99%; m/z 402.3 (MH⁺)

### 2-(4-tert-butylphenyl)-4-{[3-(1H-imidazol-1-yl)propyl]amino}-6H,7H-pyrazolo[1,5-a][1,3,5]triazin-7-one—Example 24

2-(4-tert-butylphenyl)-4-(methylsulfanyl)-6H,7H-pyrazolo[1,5-a][1,3,5]triazin-7-one (100 mg, 0.32 mmol) and 3-(1H-imidazol-1-yl)propan-1-amine (87 mg, 0.70 mmol) were dissolved in Pyridine (1.0 mL). The reaction was heated to 80° C. in a sealed tube for 4 h, then concentrated and filtered through a pad of silica gel. The filtrate was concentrated and triturated with 1:1 toluene:heptane to afford the title compound as a white solid (45 mg, 36%).

¹H NMR (500 MHz, Methanol-d4) δ 8.22 (d, J=8.6 Hz, 2H), 7.73 (s, 1H), 7.51 (d, J=8.6 Hz, 2H), 7.21 (s, 1H), 7.00 (s, 1H), 5.66 (s, 1H), 4.21 (t, J=6.8 Hz, 2H), 3.74 (t, J=6.9 Hz, 2H), 2.27 (quin, J=6.8 Hz, 2H), 1.38 (s, 9H). LCMS Method B: rt 2.25 min, 98%; m/z 392.2 (MH⁺)

### 2-(4-tert-butylphenyl)-4-{[2-(dimethylamino)ethyl]amino}-6H,7H-pyrazolo[1,5-a][1,3,5]triazin-7-one—Example 25

2-(4-tert-butylphenyl)-4-(methylsulfanyl)-6H,7H-pyrazolo[1,5-a][1,3,5]triazin-7-one (80 mg, 0.25 mmol) and (2-aminoethyl)dimethylamine (67 mg, 0.76 mmol) were dissolved in Pyridine (0.5 mL). The reaction was heated to 50° C. in a sealed tube for 18 h, then concentrated and purified using HPLC Method A, affording the title compound (36.0 mg, 38%).

¹H NMR (500 MHz, DMSO-d6) δ 8.27 (d, J=8.6 Hz, 2H), 8.15 (s, 1H), 7.91 (t, J=5.6 Hz, 1H), 7.50 (d, J=8.6 Hz, 2H), 5.69 (s, 1H), 3.70 (m, 2H), 2.64-2.59 (m, 2H), 2.26 (s, 6H), 1.32 (s, 9H). LCMS Method B: rt 2.18 min, 93%; m/z 355.2 (MH⁺)

### 2-(4-tert-butylphenyl)-4-[(2-hydroxy-2-methylpropyl)amino]-6H,7H-pyrazolo[1,5-a][1,3,5]triazin-7-one—Example 26

2-(4-tert-butylphenyl)-4-(methylsulfanyl)-6H,7H-pyrazolo[1,5-a][1,3,5]triazin-7-one (80 mg, 0.25 mmol) and 1-amino-2-methylpropan-2-ol (59 μl, 1.27 mmol) were dissolved in Pyridine (0.5 mL). The reaction was heated to 60° C. in a sealed tube for 18 h, then concentrated and purified using HPLC Method A, affording the title compound as a white solid (19.0 mg, 21%).

¹H NMR (500 MHz, Chloroform-d) δ 8.29 (d, J=8.5 Hz, 2H), 7.48 (d, J=8.6 Hz, 2H), 6.49 (t, J=6.0 Hz, 1H), 5.86 (s, 1H), 3.76 (d, J=6.2 Hz, 2H), 1.37 (s, 6H), 1.35 (s, 9H). LCMS Method B: rt 3.51 min, 100%; m/z 356.2 (MH⁺)

### 2-(4-tert-butylphenyl)-4-[(3-methoxypropyl)amino]-6H,7H-pyrazolo[1,5-a][1,3,5]triazin-7-one—Example 27

2-(4-tert-butylphenyl)-4-(methylsulfanyl)-6H,7H-pyrazolo[1,5-a][1,3,5]triazin-7-one (60 mg, 0.19 mmol) was dissolved in 3-methoxypropan-1-amine (500 μl, 4.66 mmol). The reaction mixture was heated to 130° C. in microwave for 2 h, then concentrated and purified using HPLC Method A, affording the title compound as a white solid (11.6 mg, 17%).

¹H NMR (500 MHz, DMSO-d6) δ 8.27 (d, J=8.5 Hz, 2H), 8.19 (t, J=6.1, 5.4 Hz, 1H), 7.52 (d, J=8.5 Hz, 2H), 5.68 (s, 1H), 3.66 (q, J=6.5 Hz, 2H), 3.44 (t, J=6.1 Hz, 2H), 3.26 (s, 3H), 1.93 (quin, J=6.4 Hz, 2H), 1.33 (s, 9H). LCMS Method B: rt 3.80 min, 100%; m/z 356.2 (MH+, 100%)

### 2-(4-tert-butylphenyl)-4-[(pentan-2-yl)amino]-6H,7H-pyrazolo[1,5-a][1,3,5]triazin-7-one—Example 28

2-(4-tert-butylphenyl)-4-(methylsulfanyl)-6H,7H-pyrazolo[1,5-a][1,3,5]triazin-7-one (60 mg, 0.19 mmol) and pentan-2-amine (100.17 μl, 0.95 mmol) were dissolved in 1,4-Dioxane (1 mL). The reaction mixture was heated to 130° C. in microwave for 2 h, then concentrated and purified using HPLC Method A, affording the title compound as an off-white solid (6.1 mg, 9%).

1H NMR (500 MHz, DMSO-d6) δ 10.88 (s, 1H), 8.27 (d, J=8.7 Hz, 2H), 7.94 (d, J=8.7 Hz, 1H), 7.52 (d, J=8.9 Hz, 2H), 5.69 (s, 1H), 4.46 (m, 1H), 1.80-1.71 (m, 1H), 1.61-1.52 (m, 1H), 1.39-1.31 (m, 11H), 1.29 (d, J=6.7 Hz, 3H), 0.91 (t, J=7.4 Hz, 3H). LCMS Method B: rt 4.58 min, 94%; m/z 354.3 (MH+, 100%)

### 2-(4-tert-butylphenyl)-4-[(4-phenylbutyl)amino]-6H,7H-pyrazolo[1,5-a][1,3,5]triazin-7-one—Example 29

2-(4-tert-butylphenyl)-4-(methylsulfanyl)-6H,7H-pyrazolo[1,5-a][1,3,5]triazin-7-one (60 mg, 0.19 mmol) was dissolved in 4-phenylbutan-1-amine (500 μl, 3.15 mmol). The reaction mixture was heated to 130° C. in microwave for 2 h, then concentrated and purified using HPLC Method A, affording the title compound as an off-white solid (16.1 mg, 20%).

1H NMR (500 MHz, DMSO-d6) δ 8.26 (d, J=8.7 Hz, 3H), 7.51 (d, J=8.5 Hz, 2H), 7.27-7.18 (m, 4H), 7.14 (t, J=7.1 Hz, 1H), 5.68 (s, 1H), 3.63 (m, 2H), 2.66-2.62 (m, 2H), 1.77-1.61 (m, 4H), 1.33 (s, 9H). LCMS Method B: rt 4.80 min, 100%; m/z 416.3 (MH+, 100%)

### 2-(4-tert-butylphenyl)-4-{[3-(2,3-dimethylphenoxy)propyl]amino}-6H,7H-pyrazolo[1,5-a][1,3,5]triazin-7-one—Example 30

2-(4-tert-butylphenyl)-4-(methylsulfanyl)-6H,7H-pyrazolo[1,5-a][1,3,5]triazin-7-one (100 mg, 0.32 mmol) and 3-(2,3-dimethylphenoxy)propan-1-amine (171.05 mg, 0.95 mmol) were dissolved in 1,4-Dioxane (2 mL). The reaction mixture was heated to 130° C. in microwave for 2 h, then concentrated and purified using HPLC Method A, affording the title compound (17.0 mg, 11%).

¹H NMR (500 MHz, Chloroform-d) δ 8.37-8.28 (m, 2H), 7.48-7.42 (m, 2H), 7.04 (t, J=7.9 Hz, 1H), 6.79 (d, J=7.5 Hz, 1H), 6.73 (d, J=8.2 Hz, 1H), 6.21 (t, J=5.9 Hz, 1H), 5.79 (s, 1H), 4.15 (t, J=5.7 Hz, 2H), 4.10-3.94 (m, 2H), 2.32-2.26 (m, 5H), 2.21 (s, 3H), 1.36 (s, 9H). LCMS Method B: rt 4.91 min, 95%; m/z 446.3 (MH⁺)

### 2-(4-tert-butylphenyl)-4-{[3-(cyclohexyloxy)propyl]amino}-6H,7H-pyrazolo[1,5-a][1,3,5]triazin-7-one—Example 31

2-(4-tert-butylphenyl)-4-(methylsulfanyl)-6H,7H-pyrazolo[1,5-a][1,3,5]triazin-7-one (100 mg, 0.32 mmol) and 3-(cyclohexyloxy)propan-1-amine (150 mg, 0.95 mmol) were dissolved in 1,4-Dioxane (2 mL) and DMF (0.25 mL) was added to aid heating. The reaction mixture was heated to 130° C. in microwave for 4 h, then concentrated and purified using HPLC Method A, affording the title compound (36.5 mg, 27%)

¹H NMR (500 MHz, Chloroform-d) δ 8.35 (d, J=8.5 Hz, 2H), 7.48 (d, J=8.6 Hz, 2H), 6.54 (t, J=5.4 Hz, 1H), 5.83 (s, 1H), 3.88 (q, J=6.0 Hz, 2H), 3.67 (t, J=5.7 Hz, 2H), 3.33-3.24 (m, 1H), 2.06-1.97 (m, 2H), 1.96-1.88 (m, 2H), 1.77-1.69 (m, 2H), 1.54-1.47 (m, 1H), 1.38-1.16 (m, 14H). LCMS Method B: rt 4.85 min, 99%; m/z 424.3 (MH⁺)

Example 32 was isolated as a by-product from this reaction (12.2 mg, 12%)

¹H NMR (500 MHz, DMSO-d6) δ 8.23 (d, J=8.9 Hz, 2H), 7.51 (d, J=8.9 Hz, 2H), 5.67 (s, 1H), 3.56 (s, 6H), 1.32 (s, 9H). LCMS Method B: rt 3.80 min, 100%; m/z 312.2 (MH⁺)

### 2-(4-tert-butylphenyl)-4-(dimethylamino)-6H,7H-pyrazolo[1,5-a][1,3,5]triazin-7-one—Example 32

Example 32 was isolated from the synthesis of Example 31, and can also be synthesized as follows:

2-(4-tert-butylphenyl)-4-(methylsulfanyl)-6H,7H-pyrazolo[1,5-a][1,3,5]triazin-7-one (25 mg, 0.08 mmol) was dissolved 1,4-dioxane (0.25 mL) and N-methylmethanamine (239 μl, 1M in THF) was added. The reaction mixture was heated to 130° C. for 30 min.

LCMS Method D: rt 1.43 min, 26%; m/z 311.95 (MH⁺)

### 2-(4-tert-butylphenyl)-4-[(3-phenoxypropyl)amino]-6H,7H-pyrazolo[1,5-a][1,3,5]triazin-7-one—Example 33

2-(4-tert-butylphenyl)-4-(methylsulfanyl)-6H,7H-pyrazolo[1,5-a][1,3,5]triazin-7-one (80 mg, 0.25 mmol) and 3-phenoxypropan-1-amine (115 μl, 0.76 mmol) were dissolved in 1,4-Dioxane (0.4 mL). The reaction mixture was heated to 130° C. in microwave for 2 h, then concentrated and purified using HPLC Method A, affording the title compound as an off-white solid (38.9 mg, 37%).

¹H NMR (500 MHz, DMSO-d6) δ 10.90 (br. s, 1H), 8.32 (t, J=6.1 Hz, 1H), 8.23 (d, J=8.4 Hz, 2H), 7.45 (d, J=8.3 Hz, 2H), 7.26 (dd, J=8.3, 7.5 Hz, 2H), 6.96-6.88 (m, 3H), 5.68 (s, 1H), 4.10 (t, J=6.3 Hz, 2H), 3.77 (q, J=6.5 Hz, 2H), 2.15 (quin, J=6.3 Hz, 2H), 1.31 (s, 9H). LCMS Method B: rt 4.48 min, 100%; m/z 418.3 (MH⁺)

### 4-[butyl(methyl)amino]-2-(4-tert-butylphenyl)-6H,7H-pyrazolo[1,5-a][1,3,5]triazin-7-one—Example 34

2-(4-tert-butylphenyl)-4-(methylsulfanyl)-6H,7H-pyrazolo[1,5-a][1,3,5]triazin-7-one (100 mg, 0.32 mmol) was dissolved in N-methylbutan-1-amine (500 μl, 5.74 mmol). The reaction mixture was stirred at room temperature for 12 days, then concentrated and purified using HPLC Method A, affording the title compound as an off-white solid (16.3 mg, 14%).

¹H NMR (500 MHz, Chloroform-d) δ 8.27 (d, J=8.5 Hz, 2H), 7.47 (d, J=8.5 Hz, 2H), 5.80 (s, 1H), 4.06-3.96 (m, 2H), 3.56 (s, 3H), 1.79-1.66 (m, 2H), 1.43-1.30 (m, 11H), 0.95 (t, J=7.4 Hz, 3H). LCMS Method B: rt 4.60 min, 98%; m/z 354.3 (MH⁺)

### 4-[(1-benzylpiperidin-4-yl)amino]-2-(4-tert-butylphenyl)-6H,7H-pyrazolo[1,5-a][1,3,5]triazin-7-one—Example 35

2-(4-tert-butylphenyl)-4-(methylsulfanyl)-6H,7H-pyrazolo[1,5-a][1,3,5]triazin-7-one (100 mg, 0.32 mmol) and 1-benzylpiperidin-4-amine (182 mg, 0.95 mmol) were dissolved in 1,4-Dioxane (2 mL). The reaction mixture was heated to 130° C. in microwave for 4 h, then concentrated and purified using HPLC Method A, affording the title compound (24.5 mg, 16%)

¹H NMR (500 MHz, Chloroform-d) δ 8.34 (s, 1H), 8.28 (d, J=8.5 Hz, 2H), 7.47 (d, J=8.5 Hz, 2H), 7.44-7.34 (m, 5H), 6.41-6.32 (m, 1H), 5.82 (s, 1H), 4.43-4.29 (m, 1H), 3.93 (s, 2H), 3.37-3.16 (m, 2H), 2.60-2.52 (m, 2H), 2.41-2.01 (m, 4H), 1.35 (s, 9H). LCMS Method B: rt 2.66 min, 100%; m/z 457.3 (MH⁺)

### 2-(4-tert-butylphenyl)-4-(cyclohexylamino)-6H,7H-pyrazolo[1,5-a][1,3,5]triazin-7-one—Example 36

2-(4-tert-butylphenyl)-4-(methylsulfanyl)-6H,7H-pyrazolo[1,5-a][1,3,5]triazin-7-one (80 mg, 0.25 mmol) and cyclohexanamine (76 mg, 0.76 mmol) were dissolved in 1,4-Dioxane (2 mL). The reaction mixture was heated to 130° C. in microwave for 4 h, then concentrated and purified using HPLC Method A, affording the title compound (24.7 mg, 27%)

¹H NMR (500 MHz, Chloroform-d) δ 8.35 (d, J=8.7 Hz, 2H), 7.53 (d, J=8.5 Hz, 2H), 5.88 (d, J=8.1 Hz, 1H), 5.85 (s, 1H), 4.31-4.23 (m, 1H), 2.24-2.15 (m, 2H), 1.91-1.84 (m, 2H), 1.76-1.70 (m, 2H), 1.57-1.45 (m, 4H), 1.39 (s, 9H). LCMS Method B: rt 4.67 min, 100%; m/z 366.3 (MH⁺)

Example 37 was synthesized from Example 15 as illustrated in Scheme 4.

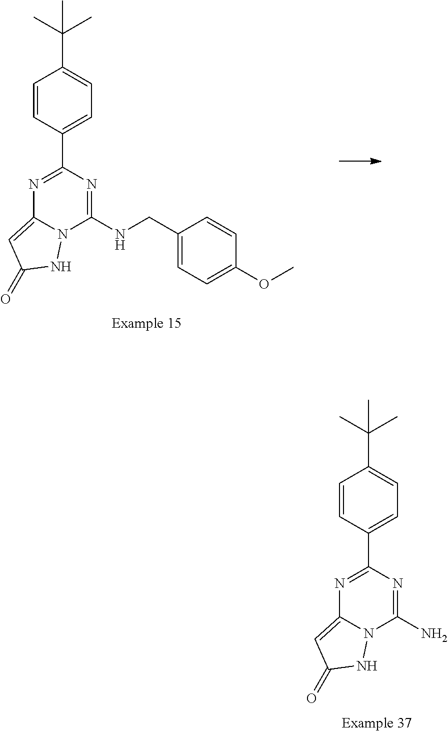 [SMILES: COc1ccc(CNc2nc(-c3ccc(C(C)(C)C)cc3)nc3cc(=O)[nH]n23)cc1 ; CC(C)(C)c1ccc(-c2nc(N)n3[nH]c(=O)cc3n2)cc1]

### 4-amino-2-(4-tert-butylphenyl)-6H,7H-pyrazolo[1,5-a][1,3,5]triazin-7-one—Example 37

2-(4-tert-butylphenyl)-4-{[(4-methoxyphenyl)methyl]amino}-6H,7H-pyrazolo[1,5-a]triazin-7-one (55 mg, 0.01 mmol) was dissolved in TFA (2 mL) and heated to 100° C. in the microwave for 5 h. The reaction mixture was evaporated to dryness and purified by column chromatography (Biotage, 10 g SNAP KP-SIL, 0-50% EtOAc in Heptane 10 CV), affording the title compound as an off-white solid (10 mg, 23%).

¹H NMR (500 MHz, Methanol-d4) δ 8.24 (d, J=8.6 Hz, 2H), 7.50 (d, J=8.6 Hz, 2H), 5.67 (s, 1H), 1.36 (s, 9H). LCMS Method B: rt 3.10 min, 91%; m/z 284.1 (MH⁺).

Example 38 was synthesized from Compound F as illustrated in Scheme 5.

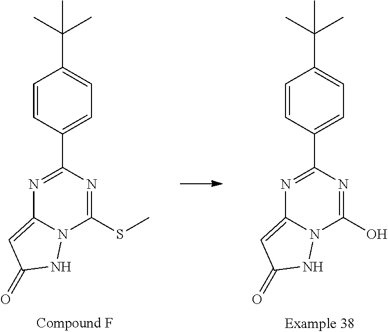 [SMILES: CSc1nc(-c2ccc(C(C)(C)C)cc2)nc2cc(=O)[nH]n12 ; CC(C)(C)c1ccc(-c2nc(O)n3[nH]c(=O)cc3n2)cc1]

### 2-(4-tert-butylphenyl)-4-hydroxy-6H,7H-pyrazolo[1,5-a][1,3,5]triazin-7-one—Example 38

2-(4-tert-butylphenyl)-4-(methylsulfanyl)-6H,7H-pyrazolo[1,5-a][1,3,5]triazin-7-one (100 mg, 0.32 mmol) was dissolved in AcOH (2 mL). H₂O₂ (30%, 108 mg, 0.95 mmol) and disodium tungstate (21 mg, 0.06 mmol) were added and the reaction mixture was stirred at room temperature for 18 h. The reaction mixture was diluted with 5% MeOH in DCM (10 mL) and washed with water (2×10 mL). The organic layer was then washed with sat. aq. NaHCO₃ (2×10 mL), dried over Na₂SO₄, filtered and evaporated to dryness. The crude product was dry-loaded onto silica and purified by column chromatography (Biotage, 10 g SNAP KP-SIL, 0-20% MeOH in TBME, 15 CV), affording the title compound as an off-white solid (6.6 mg, 7%).

¹H NMR (500 MHz, DMSO-d6) δ 8.14-7.99 (m, 2H), 7.55 (d, J=8.6 Hz, 2H), 5.77 (s, 1H), 1.32 (s, 9H). LCMS Method B: rt 2.82 min, 96%; m/z 285.1 (MH⁺).

Examples 39 to 53 were synthesized by reacting Compound F with amines (Compounds G to U) synthesized in a manner analogous to that used for Compound E (see Scheme 3).

### N-(3-aminopropyl)cyclohexanamine—Compound G

Compound G was synthesized in two steps by reacting cyclohexanamine (512 μl, 4.48 mmol) with 2-(3-bromopropyl)-2,3-dihydro-1H-isoindole-1,3-dione (1 g, 3.73 mmol) using K₂CO₃ (619 mg, 4.48 mmol) in DMF (10 mL) in a reaction analogous to that for Compound D, followed by deprotection with hydrazine hydrate (1:1) (138 μl, 2.82 mmol) in Ethanol (5 mL) in a reaction analogous to that for Compound E, yielding the title compound (80 mg, 12% over two steps)

¹H NMR (500 MHz, Chloroform-d) δ 2.74 (m, 2H), 2.67 (m, 2H), 2.39 (m, 1H), 2.15-1.95 (m, 2H), 1.84 (m, 2H), 1.68 (m, 2H), 1.61 (m, 3H), 1.28-1.10 (m, 4H), 1.09-0.95 (m, 2H).

### 2-(4-tert-butylphenyl)-4-{[3-(cyclohexylamino)propyl]amino}-6H,7H-pyrazolo[1,5-a][1,3,5]triazin-7-one—Example 39

2-(4-tert-butylphenyl)-4-(methylsulfanyl)-6H,7H-pyrazolo[1,5-a][1,3,5]triazin-7-one (70 mg, 0.22 mmol) and N-(3-aminopropyl)cyclohexanamine (80 mg, 0.49 mmol) were dissolved in Pyridine (0.5 mL). The reaction mixture was heated to 40° C. for 24 h, then concentrated and purified using HPLC Method A, affording the title compound as a salt with formic acid, an off-white solid (30.2 mg, 30%)

¹H NMR (500 MHz, Chloroform-d) δ 8.51 (s, 1H), 8.19 (d, J=8.4 Hz, 2H), 7.67 (br. s, 1H), 7.43 (d, J=8.6 Hz, 2H), 5.76 (s, 1H), 3.75 (br. s, 2H), 3.02 (t, J=6.5 Hz, 2H), 2.80 (t, J=11.3 Hz, 1H), 2.21 (m, 2H), 1.93 (d, J=10.4 Hz, 2H), 1.66 (d, J=12.9 Hz, 2H), 1.55 (d, J=13.0 Hz, 1H), 1.32 (s, 9H), 1.21 (m, 2H), 1.08 (m, 2H), 0.98 (m, 1H). LCMS Method B: rt 2.51 min, 99%; m/z 423.4 (MH⁺)

### 1-(3-aminopropyl)piperidin-4-ol—Compound H

Compound H was synthesized in two steps by reacting 4-aminocyclohexan-1-ol (0.5 g, 4.34 mmol) with 2-(3-bromopropyl)-2,3-dihydro-1H-isoindole-1,3-dione (1.16 g, 4.34 mmol) using K₂CO₃ (1.8 g, 13.02 mmol) in acetonitrile (10 mL) in a reaction analogous to that for Compound D, followed by deprotection with hydrazine hydrate (1:1) (906 μl, 18.6 mmol) in Ethanol (20 mL) in a reaction analogous to that for Compound E, yielding the title compound (590 mg, 59% over two steps)

¹H NMR (500 MHz, Methanol-d4) δ 3.72-3.55 (m, 1H), 2.87-2.76 (m, 2H), 2.65 (t, J=7.1 Hz, 2H), 2.44-2.34 (m, 2H), 2.24-2.09 (m, 2H), 1.90-1.82 (m, 2H), 1.72-1.61 (m, 2H), 1.61-1.52 (m, 2H).

### 2-(4-tert-butylphenyl)-4-{[3-(4-hydroxypiperidin-1-yl)propyl]amino}-6H,7H-pyrazolo[1,5-a][1,3,5]triazin-7-one—Example 40

2-(4-tert-butylphenyl)-4-(methylsulfanyl)-6H,7H-pyrazolo[1,5-a][1,3,5]triazin-7-one (80 mg, 0.25 mmol) and 1-(3-aminopropyl)piperidin-4-ol (121 mg, 0.76 mmol) were dissolved in Pyridine (0.5 mL). The reaction mixture was heated to 50° C. for 18 h, then concentrated and purified using HPLC Method A, affording the title compound as a salt with formic acid (50 mg, 44%)

¹H NMR (500 MHz, Methanol-d4) δ 8.42 (s, 1H), 8.32-8.21 (m, 2H), 7.62-7.46 (m, 2H), 5.69 (s, 1H), 4.56 (s, 1H), 3.88 (s, 1H), 3.84 (t, J=6.5 Hz, 2H), 3.40-3.33 (m, 2H), 3.23-3.13 (m, 2H), 3.07 (s, 2H), 2.19 (m, 2H), 1.97 (m, 2H), 1.77 (m, 2H), 1.37 (s, 9H). LCMS Method B: rt 2.15 min, 100%; m/z 425.3 (MH⁺)

### 3-(morpholin-4-yl)propan-1-amine—Compound I

Compound I was synthesized in two steps by reacting morpholine (228 μl, 2.61 mmol) with 2-(3-bromopropyl)-2,3-dihydro-1H-isoindole-1,3-dione (700 mg, 2.61 mmol) using K₂CO₃ (577 mg, 4.18 mmol) in acetonitrile (7 mL) in a reaction analogous to that for Compound D, followed by deprotection with hydrazine hydrate (1:1) (356 μl, 7.3 mmol) in Ethanol (13 mL) in a reaction analogous to that for Compound E, yielding the title compound (151 mg, 36% over two steps)

LCMS Method D: rt 0.15 min; m/z 145.0 (MH⁺)

### 2-(4-tert-butylphenyl)-4-{[3-(morpholin-4-yl)propyl]amino}-6H,7H-pyrazolo[1,5-a][1,3,5]triazin-7-one—Example 41

2-(4-tert-butylphenyl)-4-(methylsulfanyl)-6H,7H-pyrazolo[1,5-a][1,3,5]triazin-7-one (90 mg, 0.29 mmol) and 3-(morpholin-4-yl)propan-1-amine (151 mg, 1.00 mmol) were dissolved in Pyridine (0.5 mL). The reaction mixture was heated to 40° C. for 30 h, then concentrated and purified using HPLC Method A, affording the title compound as an orange tacky solid (62.5 mg, 53%)

¹H NMR (500 MHz, Chloroform-d) δ 8.35 (s, 1H), 8.27 (d, J=7.9 Hz, 2H), 7.47 (d, J=8.8 Hz, 2H), 5.78 (s, 1H), 3.93-3.87 (m, 4H), 3.82-3.75 (m, 2H), 2.99-2.79 (m, 6H), 2.15-2.07 (m, 2H), 1.35 (s, 9H). LCMS Method B: rt 2.15 min, 100%; m/z 411.3 (MH⁺)

### N-(3-aminopropyl)-2,3-dihydro-1H-inden-2-amine—Compound J

Compound J was synthesized in two steps by reacting 2,3-dihydro-1H-inden-2-amine hydrochloride (1:1) (633 mg, 3.73 mmol) with 2-(3-bromopropyl)-2,3-dihydro-1H-isoindole-1,3-dione (1.0 g, 3.73 mmol) using K₂CO₃ (1.13 g, 8.21 mmol) in DMF (10 mL) in a reaction analogous to that for Compound D, followed by deprotection with hydrazine hydrate (1:1) (131 μl, 2.7 mmol) in Ethanol (20 mL) in a reaction analogous to that for Compound E, yielding the title compound (105 mg, 80% purity, 12% over two steps)

¹H NMR (500 MHz, Chloroform-d) δ 7.23-7.19 (m, 2H), 7.18-7.14 (m, 2H), 3.65 (quin, J=6.8 Hz, 1H), 3.21 (d, J=7.1 Hz, 1H), 3.18 (d, J=7.1 Hz, 1H), 2.83-2.75 (m, 6H), 1.68 (quin, J=6.9 Hz, 2H). LCMS Method D: rt 0.15 min; m/z 191.0 (MH⁺)

### 2-(4-tert-butylphenyl)-4-({3-[(2,3-dihydro-1H-inden-2-yl)amino]propyl}amino)-6H,7H-pyrazolo[1,5-a][1,3,5]triazin-7-one—Example 42

2-(4-tert-butylphenyl)-4-(methylsulfanyl)-6H,7H-pyrazolo[1,5-a][1,3,5]triazin-7-one (50 mg, 0.16 mmol) and N-(3-aminopropyl)-2,3-dihydro-1H-inden-2-amine (105 mg, 80% purity, 0.44 mmol) were dissolved in Pyridine (0.5 mL). The reaction mixture was heated to 120° C. in a microwave for 2 h, then concentrated and purified using HPLC Method A, affording the title compound as a salt with formic acid, an orange/brown oil (6.3 mg, 8%)

¹H NMR (500 MHz, Methanol-d4) δ 8.53 (s, 1H), 8.27 (d, J=8.5 Hz, 2H), 7.53 (d, J=8.6 Hz, 2H), 7.22-7.14 (m, 4H), 5.70 (s, 1H), 4.00 (t, J=6.9 Hz, 1H), 3.89 (t, J=6.4 Hz, 2H), 3.31-3.27 (m, 2H), 3.26-3.21 (m, 2H), 3.01 (d, J=6.3 Hz, 1H), 2.98 (d, J=6.4 Hz, 1H), 2.19 (quin, J=6.7 Hz, 2H), 1.38 (s, 9H). LCMS Method B: rt 2.66 min, 97%; m/z 457.2 (MH⁺)

### N-(3-aminopropyl)aniline—Compound K

Compound K was synthesized in two steps by reacting Aniline (417 mg, 4.48 mmol) with 2-(3-bromopropyl)-2,3-dihydro-1H-isoindole-1,3-dione (1.0 g, 3.73 mmol) using K₂CO₃ (1.13 g, 8.21 mmol) in Acetonitrile (10 mL) in a reaction analogous to that for Compound D, followed by deprotection with hydrazine hydrate (1:1) (388 μl, 7.97 mmol) in Ethanol (15 mL) in a reaction analogous to that for Compound E, yielding the title compound (160 mg, 28% over two steps)

¹H NMR (500 MHz, Chloroform-d) δ 7.21-7.13 (m, 2H), 6.69 (t, J=7.3 Hz, 1H), 6.64-6.58 (m, 2H), 3.20 (t, J=6.8 Hz, 2H), 2.86 (t, J=6.7 Hz, 2H), 1.77 (quin, J=6.7 Hz, 2H).

### 2-(4-tert-butylphenyl)-4-{[3-(phenylamino)propyl]amino}-6H,7H-pyrazolo[1,5-a][1,3,5]triazin-7-one—Example 43

2-(4-tert-butylphenyl)-4-(methylsulfanyl)-6H,7H-pyrazolo[1,5-a][1,3,5]triazin-7-one (200 mg, 0.64 mmol) and N-(3-aminopropyl)aniline (160 mg, 1.07 mmol) were dissolved in Pyridine (3 mL). The reaction mixture was heated to 60° C. for 72 h, then concentrated and purified using HPLC Method A, affording the title compound as a pale brown solid (19.5 mg, 7%)

1H NMR (500 MHz, Chloroform-d) δ 8.34 (d, J=8.6 Hz, 2H), 7.49 (d, J=8.6 Hz, 2H), 7.19-7.12 (m, 2H), 6.71 (t, J=7.3 Hz, 1H), 6.63 (d, J=7.6 Hz, 2H), 6.18 (t, J=5.9 Hz, 1H), 5.83 (s, 1H), 3.91 (q, J=6.5 Hz, 2H), 3.32 (t, J=6.4 Hz, 2H), 2.08 (quin, J=6.5 Hz, 2H), 1.37 (s, 9H). LCMS Method B: rt 3.95 min, 100%; m/z 417.3 (MH⁺)

### 3-(2,3-dihydro-1H-isoindol-2-yl)propan-1-amine—Compound L

Compound L was synthesized in two steps by reacting 2,3-dihydro-1H-isoindole (203 μl, 1.79 mmol) with 2-(3-bromopropyl)-2,3-dihydro-1H-isoindole-1,3-dione (400 mg, 1.49 mmol) using K₂CO₃ (330 mg, 2.39 mmol) in Acetonitrile (6 mL) in a reaction analogous to that for Compound D, followed by deprotection with hydrazine hydrate (1:1) (150 μl, 3.07 mmol) in Ethanol (4 mL) in a reaction analogous to that for Compound E, yielding the title compound (178 mg, 64% over two steps)

¹H NMR (500 MHz, Chloroform-d) δ 7.22-7.16 (m, 4H), 3.93 (s, 4H), 2.86-2.76 (m, 4H), 1.74 (quin, J=6.9 Hz, 2H). LCMS Method D: rt 0.16 min; m/z 177.0 (MH⁺)

### 2-(4-tert-butylphenyl)-4-{[3-(2,3-dihydro-1H-isoindol-2-yl)propyl]amino}-6H,7H-pyrazolo[1,5-a][1,3,5]triazin-7-one—Example 44

2-(4-tert-butylphenyl)-4-(methylsulfanyl)-6H,7H-pyrazolo[1,5-a][1,3,5]triazin-7-one (80 mg, 0.0.25 mmol) and 3-(2,3-dihydro-1H-isoindol-2-yl)propan-1-amine (178 mg, 1.01 mmol) were dissolved in Pyridine (0.5 mL). The reaction mixture was heated to 50° C. for 18 h, then concentrated and purified using HPLC Method A, affording the title compound as a pale brown solid (44 mg, 35%)

¹H NMR (500 MHz, DMSO-d6) δ 8.29 (t, J=6.0 Hz, 1H), 8.25 (d, J=8.6 Hz, 2H), 7.43 (d, J=8.6 Hz, 2H), 7.24-7.18 (m, 4H), 5.68 (s, 1H), 3.88 (s, 4H), 3.72 (q, J=6.4 Hz, 2H), 2.78 (t, J=6.8 Hz, 2H), 1.92 (quin, J=6.7 Hz, 2H), 1.31 (s, 9H). LCMS Method B: rt 2.46 min, 100%; m/z 443.2 (MH⁺)

### 4-(4-aminobutoxy)-1-chloro-2-methylbenzene—Compound M

Compound M was synthesized in two steps by reacting 4-chloro-3-methylphenol (1 g, 7.01 mmol) with 2-(4-bromobutyl)-1H-isoindole-1,3(2H)-dione (1.8 g, 7.01 mmol) using Tetrabutyl ammonium iodide (518 mg, 1.4 mmol) and Cs₂CO₃ (4.11 g, 12.62 mmol) in THF (10 mL) in a reaction analogous to that for Compound D, followed by deprotection with hydrazine hydrate (1:1) (1.6 mL, 33.16 mmol) in Ethanol (50 mL) in a reaction analogous to that for Compound E, yielding the title compound (477 mg, 88% purity, 26% over two steps)

¹H NMR (500 MHz, Methanol-d4) δ 7.20 (d, J=8.7 Hz, 1H), 6.83 (d, J=2.9 Hz, 1H), 6.77-6.63 (m, 1H), 3.96 (t, J=6.2 Hz, 2H), 2.85-2.62 (m, 2H), 2.31 (s, 3H), 1.89-1.72 (m, 2H), 1.74-1.56 (m, 2H). LCMS Method A: rt 1.53 min, 88%; m/z 214.2 (MH⁺)

### 2-(4-tert-butylphenyl)-4-{[4-(4-chloro-3-methylphenoxy)butyl]amino}-6H,7H-pyrazolo[1,5-a][1,3,5]triazin-7-one—Example 45

2-(4-tert-butylphenyl)-4-(methylsulfanyl)-6H,7H-pyrazolo[1,5-a][1,3,5]triazin-7-one (50 mg, 0.16 mmol) and 4-(4-aminobutoxy)-1-chloro-2-methylbenzene (30 mg, 0.16 mmol) were dissolved in Acetonitrile (1 mL). The reaction mixture was heated to 130° C. in a microwave for 4.5 h, then concentrated and purified by column chromatography (Biotage, 10 g SNAP KP-SIL, 25-100% EtOAc in heptane, 10 CV). This was followed by filtration through a 2 g SCX-2 column, washing with methanol and eluting with 0.7M NH₃ in methanol. The basic eluent was concentrated, affording the title compound (25.7 mg, 34%)

¹H NMR (500 MHz, Chloroform-d) δ 8.28 (d, J=8.5 Hz, 2H), 7.41 (d, J=8.5 Hz, 2H), 7.17 (m, 1H), 6.75 (d, J=2.8 Hz, 1H), 6.64 (m, 1H), 5.81 (m, 2H), 3.97 (m, 2H), 3.88-3.65 (m, 2H), 2.28 (s, 3H), 1.88 (br s, 4H), 1.33 (s, 9H). LCMS Method E: rt 4.81 min, 100%; m/z 480.1 (MH⁺)

### 4-(2-aminoethoxy)-1-chloro-2-methylbenzene—Compound N

Compound N was synthesized in two steps by reacting 4-chloro-3-methylphenol (1 g, 7.01 mmol) with 2-(2-bromoethyl)-2,3-dihydro-1H-isoindole-1,3-dione (1.96 g, 7.71 mmol) using Tetrabutyl ammonium iodide (518 mg, 1.4 mmol) and Cs₂CO₃ (4.11 g, 12.62 mmol) in THF (10 mL) in a reaction analogous to that for Compound D, followed by deprotection with hydrazine hydrate (1:1) (232 μl, 4.75 mmol) in Ethanol (2 mL) in a reaction analogous to that for Compound E, yielding the title compound (175 mg, 85% purity, 10% over two steps)

¹H NMR (500 MHz, Methanol-d4) δ 7.21 (d, J=8.8 Hz, 1H), 6.88 (d, J=2.9 Hz, 1H), 6.81-6.71 (m, 1H), 3.98 (t, J=5.3 Hz, 2H), 2.99 (t, J=5.3 Hz, 2H), 2.31 (s, 3H). LCMS Method A: rt 1.39 min, 85%; m/z 186.1 (MH⁺)

### 2-(4-tert-butylphenyl)-4-{[2-(4-chloro-3-methylphenoxy)ethyl]amino}-6H,7H-pyrazolo[1,5-a][1,3,5]triazin-7-one—Example 46

2-(4-tert-butylphenyl)-4-(methylsulfanyl)-6H,7H-pyrazolo[1,5-a][1,3,5]triazin-7-one (80 mg, 0.25 mmol) and 4-(2-aminoethoxy)-1-chloro-2-methylbenzene (177 mg, 85% purity, 0.81 mmol) were dissolved in Acetonitrile (3 mL). The reaction mixture was heated to 150° C. in a microwave for 11 h, then concentrated and purified by column chromatography (Biotage, 10 g SNAP KP-SIL, 0-100% EtOAc in heptane, 10 CV), affording the title compound (9.1 mg, 8%)

¹H NMR (500 MHz, Chloroform-d) δ 8.33 (d, J=8.6 Hz, 2H), 7.49 (d, J=8.6 Hz, 2H), 7.24 (m, 2H), 6.82 (d, J=2.8 Hz, 1H), 6.73 (m, 1H), 6.45 (m, 1H), 5.84 (s, 1H), 4.24 (t, J=5.1 Hz, 2H), 4.15 (q, J=5.4 Hz, 2H), 2.33 (s, 3H), 1.37 (s, 9H). LCMS Method B: rt 4.80 min, 100%; m/z 452.2 (MH⁺)

### N-(3-aminopropyl)-4-chloro-3-methylaniline—Compound O

Compound O was synthesized in two steps by reacting 4-chloro-3-methylaniline (0.5 g, 3.53 mmol) with 2-(3-bromopropyl)-1H-isoindole-1,3(2H)-dione (1.04 g, 3.88 mmol) using triethylamine (0.98 mL, 7.1 mmol) in Acetonitrile (10 mL) in a reaction analogous to that for Compound D, followed by deprotection with hydrazine hydrate (1:1) (0.23 mL, 4.68 mmol) in Ethanol (10 mL) in a reaction analogous to that for Compound E, yielding the title compound (166 mg, 23% over two steps)

¹H NMR (500 MHz, Methanol-d4) δ 7.02 (d, J=8.6 Hz, 1H), 6.53 (d, J=2.7 Hz, 1H), 6.42 (dd, J=8.6, 2.8 Hz, 1H), 3.10 (t, J=6.9 Hz, 2H), 2.83-2.66 (m, 2H), 2.24 (s, 3H), 1.76 (quin, J=7.0 Hz, 2H). LCMS Method A: rt 1.95 min, 98%; m/z 197.2 (MH⁺)

### 2-(4-tert-butylphenyl)-4-({3-[(4-chloro-3-methylphenyl)amino]propyl}amino)-6H,7H-pyrazolo[1,5-a][1,3,5]triazin-7-one—Example 47

2-(4-tert-butylphenyl)-4-(methylsulfanyl)-6H,7H-pyrazolo[1,5-a][1,3,5]triazin-7-one (100 mg, 0.32 mmol) and N-(3-aminopropyl)-4-chloro-3-methylaniline (190 mg, 0.95 mmol) were dissolved in 1,4-Dioxane (4 mL). The reaction mixture was heated to 130° C. in a microwave for 11 h, then concentrated and purified using HPLC Method A, affording the title compound (7.9 mg, 5%)

¹H NMR (500 MHz, Chloroform-d) δ 8.29 (d, J=8.4 Hz, 2H), 7.47 (d, J=8.5 Hz, 2H), 7.04 (d, J=8.6 Hz, 1H), 6.45-6.37 (m, 2H), 6.37-6.25 (m, 1H), 5.81 (s, 1H), 3.86-3.79 (m, 2H), 3.20 (t, J=6.3 Hz, 2H), 2.21 (s, 3H), 2.04-1.90 (m, 2H), 1.36 (s, 9H). LCMS Method B: rt 4.66 min, 94%; m/z 465.3 (MH⁺)

### 4-(3-aminopropoxy)-1-fluoro-2-methylbenzene—Compound P

Compound P was synthesized in two steps by reacting 4-fluoro-3-methylphenol (415 μl, 3.73 mmol) with 2-(3-bromopropyl)-1H-isoindole-1,3(2H)-dione (1 g, 3.73 mmol) using Tetrabutyl ammonium iodide (0.25 g, 0.68 mmol) and Cs₂CO₃ (1.99 g, 6.1 mmol) in THF (10 mL) in a reaction analogous to that for Compound D, followed by deprotection with hydrazine hydrate (1:1) (1.02 mL, 20.99 mmol) in Ethanol (2 mL) in a reaction analogous to that for Compound E, yielding the title compound (540 mg, 90% purity, 71% over two steps)

¹H NMR (500 MHz, Methanol-d4) δ 7.03-6.84 (m, 1H), 6.83-6.73 (m, 1H), 6.73-6.62 (m, 1H), 4.00 (t, J=6.1 Hz, 2H), 2.88 (t, J=7.0 Hz, 2H), 2.22 (d, J=1.7 Hz, 3H), 1.96-1.91 (m, 2H).

### 2-(4-tert-butylphenyl)-4-{[3-(4-fluoro-3-methylphenoxy)propyl]amino}-6H,7H-pyrazolo[1,5-a][1,3,5]triazin-7-one—Example 48

2-(4-tert-butylphenyl)-4-(methylsulfanyl)-6H,7H-pyrazolo[1,5-a][1,3,5]triazin-7-one (100 mg, 0.32 mmol) and 4-(3-aminopropoxy)-1-fluoro-2-methylbenzene (175 mg, 0.95 mmol) were dissolved in 1,4-Dioxane (4 mL). The reaction mixture was heated to 130° C. in a microwave for 9 h, then concentrated and purified using HPLC Method A, affording the title compound (11.4 mg, 8%)

¹H NMR (500 MHz, Chloroform-d) δ 8.33 (d, J=8.5 Hz, 2H), 7.46 (d, J=8.5 Hz, 2H), 6.87 (t, J=9.0 Hz, 1H), 6.77-6.70 (m, 1H), 6.70-6.63 (m, 1H), 6.24 (t, J=5.5 Hz, 1H), 5.79 (s, 1H), 4.11 (t, J=5.7 Hz, 2H), 3.98 (q, J=6.2 Hz, 2H), 2.31-2.21 (m, 2H), 2.19 (d, J=1.5 Hz, 3H), 1.36 (s, 9H). LCMS Method B: rt 4.67 min, 98%; m/z 450.3 (MH⁺)

### 4-(3-aminopropoxy)-1,2-dimethylbenzene—Compound Q

Compound Q was synthesized in two steps by reacting 3,4-dimethylphenol (456 mg, 3.73 mmol) with 2-(3-bromopropyl)-1H-isoindole-1,3(2H)-dione (1 g, 3.73 mmol) using Tetrabutyl ammonium iodide (0.25 g, 0.68 mmol) and Cs₂CO₃ (1.99 g, 6.1 mmol) in THF (10 mL) in a reaction analogous to that for Compound D, followed by deprotection with hydrazine hydrate (1:1) (0.86 mL, 17.69 mmol) in Ethanol (20 mL) in a reaction analogous to that for Compound E, yielding the title compound (251 mg, 80% purity, 30% over two steps)

¹H NMR (500 MHz, Methanol-d4) δ 6.99 (d, J=8.3 Hz, 1H), 6.70 (d, J=2.4 Hz, 1H), 6.67-6.54 (m, 1H), 3.99 (t, J=6.2 Hz, 2H), 2.82 (t, J=7.0 Hz, 2H), 2.21 (s, 3H), 2.17 (s, 3H), 1.92-1.87 (m, 2H). LCMS Method D: rt 0.83 min, 95%; m/z 180.0 (MH⁺)

### 2-(4-tert-butylphenyl)-4-{[3-(3,4-dimethylphenoxy)propyl]amino}-6H,7H-pyrazolo[1,5-a][1,3,5]triazin-7-one—Example 49

2-(4-tert-butylphenyl)-4-(methylsulfanyl)-6H,7H-pyrazolo[1,5-a][1,3,5]triazin-7-one (100 mg, 0.32 mmol) and 4-(3-aminopropoxy)-1,2-dimethylbenzene (214 mg, 80% purity, 0.95 mmol) were dissolved in 1,4-Dioxane (2 mL). The reaction mixture was heated to 130° C. in a microwave for 4 h, then concentrated and purified using HPLC Method A, affording the title compound (32 mg, 21%)

¹H NMR (500 MHz, Chloroform-d) 8.34 (d, J=8.6 Hz, 2H), 7.47 (d, J=8.6 Hz, 2H), 7.04-6.98 (m, 1H), 6.77 (d, J=2.5 Hz, 1H), 6.74-6.64 (m, 1H), 6.47-6.37 (m, 1H), 5.81 (s, 1H), 4.14 (t, J=5.7 Hz, 2H), 3.97 (q, J=6.3 Hz, 2H), 2.27-2.22 (m, 2H), 2.21 (s, 3H), 2.18 (s, 3H), 1.36 (s, 9H). LCMS Method B: rt 4.83 min, 99%; m/z 446.3 (MH⁺)

### 2-(3-aminopropoxy)pyrazine—Compound R

Compound R was synthesized in two steps by reacting pyrazin-2-ol (639 mg, 6.65 mmol) with 2-(3-bromopropyl)-1H-isoindole-1,3(2H)-dione (1.78 g, 6.65 mmol) using Tetrabutyl ammonium iodide (0.45 g, 1.21 mmol) and Cs₂CO₃ (3.54 g, 10.88 mmol) in THF (10 mL) in a reaction analogous to that for Compound D, followed by separation of isomers by column chromatography (Biotage, 25 g SNAP KP-SIL, 50-100% EtOAc in heptane, 10 CV), and deprotection with hydrazine hydrate (1:1) (0.26 mL, 5.41 mmol) in Ethanol (10 mL) in a reaction analogous to that for Compound E, yielding the title compound (131 mg, 12% over two steps)

¹H NMR (500 MHz, DMSO-d6) δ 8.29 (s, 1H), 8.19 (m, 2H), 4.36 (t, J=6.5 Hz, 2H), 2.70 (t, J=6.8 Hz, 2H), 1.82 (quin, J=6.6 Hz, 2H). LCMS Method D: rt 0.19 min; m/z 153.9 (MH⁺)

### 2-(4-tert-butylphenyl)-4-{[3-(pyrazin-2-yloxy)propyl]amino}-6H,7H-pyrazolo[1,5-a][1,3,5]triazin-7-one—Example 50

2-(4-tert-butylphenyl)-4-(methylsulfanyl)-6H,7H-pyrazolo[1,5-a][1,3,5]triazin-7-one (85 mg, 0.27 mmol) and 2-(3-aminopropoxy)pyrazine (131 mg, 0.87 mmol) were dissolved in 1,4-Dioxane (1 mL). The reaction mixture was heated to 130° C. in a microwave for 4 h, then concentrated and purified using HPLC Method A, affording the title compound as a white solid (12.5 mg, 11%)

¹H NMR (500 MHz, DMSO-d6) δ 8.33 (t, J=6.2, 5.4 Hz, 1H), 8.30 (d, J=1.2 Hz, 1H), 8.19 (t, J=8.6 Hz, 2H), 8.16-8.12 (m, 2H), 7.45 (d, J=8.3 Hz, 2H), 5.68 (s, 1H), 4.42 (t, J=5.7 Hz, 2H), 3.79 (q, J=6.5 Hz, 2H), 2.18 (quin, J=6.5 Hz, 2H), 1.32 (s, 9H). LCMS Method B: rt 3.84 min, 97%; m/z 420.3 (MH⁺)

### 1-(3-aminopropyl)-1,2-dihydropyrazin-2-one—Compound S

Compound S was synthesized in two steps by reacting pyrazin-2-ol (639 mg, 6.65 mmol) with 2-(3-bromopropyl)-1H-isoindole-1,3(2H)-dione (1.78 g, 6.65 mmol) using Tetrabutyl ammonium iodide (0.45 g, 1.21 mmol) and Cs₂CO₃ (3.54 g, 10.88 mmol) in THF (10 mL) in a reaction analogous to that for Compound D, followed by separation of isomers by column chromatography (Biotage, 25 g SNAP KP-SIL, 50-100% EtOAc in heptane, 10 CV), and deprotection with hydrazine hydrate (1:1) (0.405 mL, 8.31 mmol) in Ethanol (10 mL) in a reaction analogous to that for Compound E, yielding the title compound (160 mg, 11% over two steps)

¹H NMR (500 MHz, Chloroform-d) δ 8.17 (d, J=0.9 Hz, 1H), 7.34 (d, J=4.3 Hz, 1H), 7.17 (dd, J=4.4, 0.9 Hz, 1H), 4.05 (t, J=6.9 Hz, 2H), 2.77 (t, J=6.5 Hz, 2H), 1.91 (quin, J=6.7 Hz, 2H). LCMS Method D: rt 0.16 min; m/z 154.0 (MH⁺)

### 1-(3-{[2-(4-tert-butylphenyl)-7-oxo-6H,7H-pyrazolo[1,5-a][1,3,5]triazin-4-yl]amino}propyl)-1,2-dihydropyrazin-2-one—Example 51

2-(4-tert-butylphenyl)-4-(methylsulfanyl)-6H,7H-pyrazolo[1,5-a][1,3,5]triazin-7-one (88 mg, 0.28 mmol) and 1-(3-aminopropyl)-1,2-dihydropyrazin-2-one (160 mg, 0.83 mmol) were dissolved in 1,4-Dioxane (3 mL). The reaction mixture was heated to 130° C. in a microwave for 20 min, then concentrated and purified using HPLC Method A, affording the title compound as an off-white solid (1.7 mg, 1%)

¹H NMR (500 MHz, Chloroform-d) δ 8.36 (s, 1H), 8.19 (d, J=8.0 Hz, 2H), 7.37 (d, J=8.0 Hz, 2H), 7.30 (d, J=3.8 Hz, 1H), 7.25-7.19 (m, 1H), 7.08 (d, J=2.8 Hz, 1H), 5.75 (s, 1H), 4.01 (m, 2H), 3.68-3.59 (m, 2H), 2.12-2.03 (m, 2H), 1.25 (s, 9H). LCMS Method B: rt 3.19 min, 90%; m/z 420.3 (MH⁺)

### 5-(3-aminopropoxy)-2-chloropyrimidine—Compound T

Compound T was synthesized in two steps by reacting 2-chloropyrimidin-5-ol (487 mg, 3.73 mmol) with 2-(3-bromopropyl)-1H-isoindole-1,3(2H)-dione (1 g, 3.73 mmol) using Tetrabutyl ammonium iodide (0.25 g, 0.68 mmol) and Cs₂CO₃ (1.99 g, 6.1 mmol) in THF (10 mL) in a reaction analogous to that for Compound D, followed by deprotection with hydrazine hydrate (1:1) (0.29 mL, 6.06 mmol) in Ethanol (2 mL) in a reaction analogous to that for Compound E, yielding the title compound (203 mg, 90% purity, 23% over two steps)

¹H NMR (500 MHz, Chloroform-d) δ 8.23 (s, 2H), 4.10 (t, J=6.1 Hz, 2H), 2.86 (t, J=6.7 Hz, 2H), 1.90 (quin, J=6.4 Hz, 2H).

### 2-(4-tert-butylphenyl)-4-({3-[(2-chloropyrimidin-5-yl)oxy]propyl}amino)-6H,7H-pyrazolo[1,5-a][1,3,5]triazin-7-one—Example 52

2-(4-tert-butylphenyl)-4-(methylsulfanyl)-6H,7H-pyrazolo[1,5-a][1,3,5]triazin-7-one (169 mg, 0.54 mmol) and 5-(3-aminopropoxy)-2-chloropyrimidine (203 mg, 0.97 mmol) were dissolved in 1,4-Dioxane (3 mL). The reaction mixture was heated to 130° C. in a microwave for 6 h, then concentrated and purified using HPLC Method A, affording the title compound as a white solid (48.5 mg, 20%)

¹H NMR (500 MHz, DMSO-d6) δ 8.51 (s, 2H), 8.36 (t, J=6.2 Hz, 1H), 8.16 (d, J=8.3 Hz, 2H), 7.41 (d, J=8.3 Hz, 2H), 5.69 (s, 1H), 4.26 (t, J=5.7 Hz, 2H), 3.78 (q, J=6.4 Hz, 2H), 2.16 (quin, J=6.2 Hz, 2H), 1.32 (s, 9H). LCMS Method B: rt 3.98 min, 100%; m/z 454.2 (MH⁺)

### 3-(3-aminopropoxy)-2,4-dimethylpyridine—Compound U

Compound U was synthesized in two steps by reacting 2,4-dimethylpyridin-3-ol (459 mg, 3.73 mmol) with 2-(3-bromopropyl)-1H-isoindole-1,3(2H)-dione (1 g, 3.73 mmol) using Tetrabutyl ammonium iodide (0.25 g, 0.68 mmol) and Cs₂CO₃ (1.99 g, 6.1 mmol) in THF (10 mL) in a reaction analogous to that for Compound D, followed by deprotection with hydrazine hydrate (1:1) (0.78 mL, 15.91 mmol) in Ethanol (3 mL) in a reaction analogous to that for Compound E, yielding the title compound (289 mg, 90% purity, 34% over two steps)

¹H NMR (500 MHz, DMSO-d6) δ 8.05 (d, J=4.8 Hz, 1H), 7.06 (d, J=4.8 Hz, 1H), 3.83 (t, J=6.4 Hz, 2H), 2.76 (t, J=6.7 Hz, 2H), 2.40 (s, 3H), 2.24 (s, 3H), 1.81 (t, J=6.6 Hz, 2H).

### 2-(4-tert-butylphenyl)-4-({3-[(2,4-dimethylpyridin-3-yl)oxy]propyl}amino)-6H,7H-pyrazolo[1,5-a][1,3,5]triazin-7-one—Example 53

2-(4-tert-butylphenyl)-4-(methylsulfanyl)-6H,7H-pyrazolo[1,5-a][1,3,5]triazin-7-one (151 mg, 0.48 mmol) and 3-(3-aminopropoxy)-2,4-dimethylpyridine (289 mg, 1.45 mmol) were dissolved in 1,4-Dioxane (3 mL). The reaction mixture was heated to 130° C. in a microwave for 6 h, then concentrated and purified using HPLC Method A, followed by trituration with TBME, affording the title compound as an orange/brown solid (19.6 mg, 8%)

¹H NMR (500 MHz, DMSO-d6) δ 8.32 (t, J=4.9 Hz, 1H), 8.28 (d, J=8.2 Hz, 2H), 8.06 (d, J=4.9 Hz, 1H), 7.50 (d, J=8.4 Hz, 2H), 7.07 (d, J=4.5 Hz, 1H), 5.71 (s, 1H), 3.93 (t, J=6.2 Hz, 2H), 3.84 (q, J=6.5 Hz, 2H), 2.40 (s, 3H), 2.24 (s, 3H), 2.23-2.18 (m, 2H), 1.32 (s, 9H). LCMS Method B: rt 2.62 min, 90%; m/z 447.3 (MH⁺)

Example 54 was synthesised by reacting Compound F with Compound W. The synthetic route for Compound W is illustrated in Scheme 6.

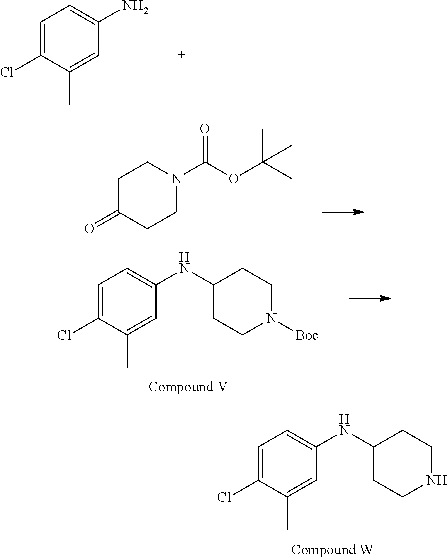

### tert-butyl 4-[(4-chloro-3-methylphenyl)amino]piperidine-1-carboxylate—Compound V

tert-butyl 4-oxopiperidine-1-carboxylate (1 g, 5.02 mmol) and 4-chloro-3-methylaniline (711 mg, 5.02 mmol), were dissolved in DCE (30 mL) and acetic acid (287 μl, 5.02 mmol) was added. The reaction was stirred at 60° C. for 6 h. The reaction was allowed to cool and Sodium Tri(acetoxy)borohydride (1.49 g, 7.03 mmol) was added. The reaction was then stirred at room temperature for 18 h, then washed with water and brine. The organic layer was dried and concentrated, then purified using column chromatography (Biotage, 25 g SNAP KP-SIL, 100% DCM, 10 CV). The crude product was then purified a second time using low pH reverse phase column chromatography to yield a white solid (130 mg, 8%)

LCMS Method D: rt 1.64 min, 92%; m/z 269.1 (MH⁺-^{t}Bu)

### N-(4-chloro-3-methylphenyl)piperidin-4-amine—Compound W

tert-butyl 4-[(4-chloro-3-methylphenyl)amino]piperidine-1-carboxylate (130 mg, 0.4 mmol) was dissolved in DCM (1 mL) and TFA (1 mL). The reaction was stirred at room temperature for 1 h. The solution was evaporated to dryness, re-dissolved in DCM and washed with sodium carbonate. The organics were separated using a hydrophobic frit and concentrated under vacuum to yield a brown tacky solid (88 mg, 97%).

¹H NMR (500 MHz, Chloroform-d) δ 7.09 (d, J=8.6 Hz, 1H), 6.46 (d, J=3.1 Hz, 1H), 6.38 (dd, J=8.6, 3.4 Hz, 1H), 3.46 (s, 1H), 3.31 (m, 1H), 3.10 (dt, J=12.9, 3.6 Hz, 2H), 2.71 (t, J=11.6 Hz, 2H), 2.28 (s, 3H), 2.03 (d, J=12.0 Hz, 2H), 1.29 (qd, J=11.5, 3.9 Hz, 2H). LCMS Method D: rt 0.91 min, 92%; m/z 225.0 (MH⁺)

### 2-(4-tert-butylphenyl)-4-{4-[(4-chloro-3-methylphenyl)amino]piperidin-1-yl}-6H,7H-pyrazolo[1,5-a][1,3,5]triazin-7-one—Example 54

2-(4-tert-butylphenyl)-4-(methylsulfanyl)-6H,7H-pyrazolo[1,5-a][1,3,5]triazin-7-one (70 mg, 0.22 mmol) and N-(4-chloro-3-methylphenyl)piperidin-4-amine (88 mg, 0.37 mmol) were dissolved in 1,4-Dioxane (0.5 mL). The reaction mixture was heated to 130° C. in a microwave for 6 h, then concentrated and purified using HPLC Method A, affording the title compound as an off-white solid (32.5 mg, 29%)

¹H NMR (500 MHz, DMSO-d6) δ 8.31 (d, J=8.8 Hz, 2H), 7.51 (d, J=8.8 Hz, 2H), 7.14 (d, J=8.6 Hz, 1H), 6.53 (d, J=2.7 Hz, 1H), 6.44 (dd, J=8.6, 2.7 Hz, 1H), 5.87 (s, 1H), 5.22 (d, J=13.5 Hz, 2H), 3.68-3.60 (m, 1H), 3.53-3.45 (m, 2H), 2.33 (s, 3H), 2.29-2.24 (m, 2H), 1.38 (s, 9H). N.b. 2 protons obscured by H₂O signal. LCMS Method B: rt 4.98 min, 100%; m/z 491.3 (MH⁺)

Examples 55 and 56 were synthesized by reacting Compound F with amines (Compounds X and Y) synthesized in a manner analogous to that used for Compound W

### 1-[(2-methyl-1H-imidazol-5-yl)methyl]piperidin-4-amine—Compound X

Compound X was synthesized in two steps by reacting tert-butyl N-(piperidin-4-yl)carbamate (300 mg, 1.5 mmol) with 2-methyl-1H-imidazole-5-carbaldehyde (165 mg, 1.5 mmol) using Sodium Tri(acetoxy)borohydride (445 mg, 2.1 mmol) and acetic acid (86 μl, 1.5 mmol) in DCE (8 mL) in a reaction analogous to that for Compound V, followed by deprotection with TFA (2 mL) in a reaction analogous to that for Compound W, yielding the title compound as a pale orange solid (226 mg, 63% over two steps)

¹H NMR (500 MHz, Chloroform-d) δ 6.73 (s, 1H), 3.74 (s, 2H), 3.09 (dt, J=12.7, 3.2 Hz, 2H), 2.60 (td, J=12.4, 2.5 Hz, 3H), 2.38 (s, 3H), 1.91 (d, J=10.6 Hz, 2H), 1.26 (qd, J=12.2, 4.0 Hz, 2H). LCMS Method A: rt 1.14 min, 100%; m/z 195.2 (MH⁺)

### 2-(4-tert-butylphenyl)-4-({1-[(2-methyl-1H-imidazol-5-yl)methyl]piperidin-4-yl}amino)-6H,7H-pyrazolo[1,5-a][1,3,5]triazin-7-one—Example 55

2-(4-tert-butylphenyl)-4-(methylsulfanyl)-6H,7H-pyrazolo[1,5-a][1,3,5]triazin-7-one (80 mg, 0.25 mmol) and 1-[(2-methyl-1H-imidazol-5-yl)methyl]piperidin-4-amine (113 mg, 0.55 mmol) were dissolved in Pyridine (0.5 mL). The reaction mixture was heated to 60° C. for 42 h, then concentrated and purified using HPLC Method A, affording the title compound as an orange tacky solid (32 mg, 27%)

¹H NMR (500 MHz, DMSO-d6) δ 8.28-8.23 (m, 3H), 7.51 (d, J=8.6 Hz, 2H), 6.81 (s, 1H), 5.72 (s, 1H), 5.25-5.16 (m, 2H), 3.74 (s, 2H), 3.46-3.39 (m, 2H), 3.01-2.92 (m, 2H), 2.55 (s, 1H), 2.25 (s, 3H), 2.10-2.03 (m, 2H), 1.54-1.45 (m, 2H), 1.33 (s, 9H). LCMS Method B: rt 1.82 min, 99%; m/z 461.3 (MH⁺)

### 1-N-benzylcyclohexane-1,4-diamine—Compound Y

Compound Y was synthesized in two steps by reacting tert-butyl N-(4-aminocyclohexyl)carbamate (500 mg, 2.33 mmol) with benzaldehyde (238 μl, 2.33 mmol) using Sodium Tri(acetoxy)borohydride (742 mg, 3.5 mmol) and acetic acid (134 μl, 2.33 mmol) in DCE (20 mL) in a reaction analogous to that for Compound V, followed by deprotection with TFA (1 mL) and DCM (4 mL) in a reaction analogous to that for Compound W, yielding the title compound as a pale orange solid (328 mg, 68% over two steps)

LCMS Method D: rt 0.16 min; m/z 205.0 (MH⁺)

### 4-{[4-(benzylamino)cyclohexyl]amino}-2-(4-tert-butylphenyl)-6H,7H-pyrazolo[1,5-a][1,3,5]triazin-7-one—Example 56

2-(4-tert-butylphenyl)-4-(methylsulfanyl)-6H,7H-pyrazolo[1,5-a][1,3,5]triazin-7-one (200 mg, 0.64 mmol) and 1-N-benzylcyclohexane-1,4-diamine (328 mg, 1.61 mmol) were dissolved in Pyridine (4 mL). The reaction mixture was heated to 120° C. in a microwave for 24 h, then concentrated and purified using HPLC Method A, affording the title compound as an orange tacky solid (15.4 mg, 5%)

¹H NMR (500 MHz, Methanol-d4) δ 8.34 (s, 2H), 8.26 (d, J=8.5 Hz, 2H), 7.59-7.41 (m, 7H), 5.69 (s, 1H), 4.29 (s, 3H), 3.26 (t, J=11.1 Hz, 1H), 2.35 (m, 4H), 1.69 (tt, J=24.6, 11.8 Hz, 4H), 1.37 (s, 9H). LCMS Method B: rt 2.56 min, 100%; m/z 471.3 (MH⁺)

Example 57 was synthesised by reacting Compound F with Compound AA. The synthetic route for Compound AA is illustrated in Scheme 7.

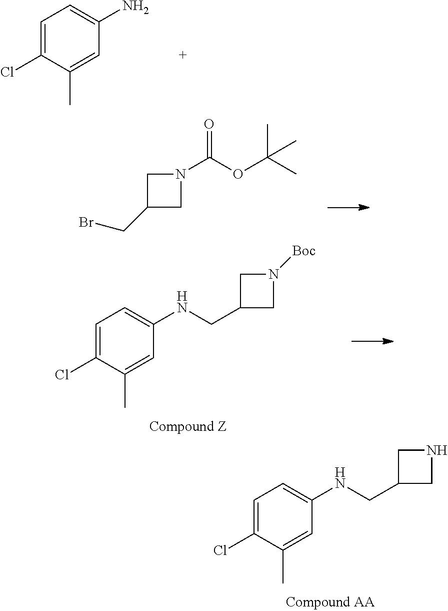

### tert-butyl 3-{[(4-chloro-3-methylphenyl)amino]methyl}azetidine-1-carboxylate—Compound Z

4-chloro-3-methylaniline (306 mg, 2.16 mmol) and tert-butyl 3-(bromomethyl)azetidine-1-carboxylate (450 mg, 1.8 mmol) were stirred in acetonitrile (5 mL) under nitrogen, and KI (60 mg, 0.36 mmol) and K₂CO₃ (298 mg, 2.16 mmol) were added. The reaction was stirred at 85° C. for 48 h. The reaction was concentrated and partitioned between ethyl acetate and saturated sodium carbonate. The combined organics were dried over Na₂SO₄, and concentrated. The crude product was purified by reverse phase column chromatography to yield the title compound as a white solid (311 mg, 54%)

¹H NMR (500 MHz, Chloroform-d) 57.13 (d, J=8.6 Hz, 1H), 6.49 (d, J=2.8 Hz, 1H), 6.40 (dd, J=8.6, 2.8 Hz, 1H), 4.07 (t, J=8.4 Hz, 2H), 3.68 (dd, J=8.7, 5.1 Hz, 2H), 3.33 (d, J=7.3 Hz, 2H), 2.83-2.75 (m, 1H), 2.32 (s, 3H), 1.46 (s, 9H). LCMS Method D: rt 1.52 min, 97%; m/z 254.95 (MH⁺-^{t}Bu)

### N-(azetidin-3-ylmethyl)-4-chloro-3-methylaniline—Compound AA

tert-butyl 3-{[(4-chloro-3-methylphenyl)amino]methyl}azetidine-1-carboxylate (311 mg, 1.0 mmol) was stirred in TFA (2 mL) at room temperature for 3 h. The reaction mixture was then concentrated, and purified using an SCX-II column, using methanol to elute the impurities and 0.7M NH₃ in methanol to elute the product. The basic eluent was concentrated to yield the title compound as a yellow oil (128 mg, 61%)

¹H NMR (500 MHz, Chloroform-d) δ 7.10 (d, J=8.6 Hz, 1H), 6.47 (d, J=2.4 Hz, 1H), 6.38 (dd, J=8.6, 2.6 Hz, 1H), 3.79 (t, J=7.7 Hz, 2H), 3.41 (t, J=6.7 Hz, 2H), 3.31 (d, J=6.8 Hz, 2H), 2.95 (dt, J=13.4, 6.7 Hz, 1H), 2.29 (s, 3H).

### 2-(4-tert-butylphenyl)-4-(3-{[(4-chloro-3-methylphenyl)amino]methyl}azetidin-1-yl)-6H,7H-pyrazolo[1,5-a][1,3,5]triazin-7-one—Example 57

2-(4-tert-butylphenyl)-4-(methylsulfanyl)-6H,7H-pyrazolo[1,5-a][1,3,5]triazin-7-one (80 mg, 0.25 mmol) and N-(azetidin-3-ylmethyl)-4-chloro-3-methylaniline (128 mg, 0.58 mmol) were dissolved in Pyridine (0.5 mL). The reaction mixture was heated to 40° C. for 18 h, then concentrated and purified using HPLC Method A, affording the title compound as an orange tacky solid (60.7 mg, 50%)

¹H NMR (500 MHz, Chloroform-d) δ 8.22 (d, J=8.5 Hz, 2H), 8.09 (s, 1H), 7.45 (d, J=8.6 Hz, 2H), 7.10 (d, J=8.6 Hz, 1H), 6.49 (d, J=2.7 Hz, 1H), 6.40 (dd, J=8.6, 2.8 Hz, 1H), 5.77 (s, 1H), 4.64 (t, J=8.5 Hz, 2H), 4.34-4.23 (m, 2H), 3.37 (d, J=7.2 Hz, 2H), 2.99 (m, 1H), 2.28 (s, 3H), 1.34 (s, 9H). LCMS Method B: rt 4.81 min, 99%; m/z 477.2 (MH⁺)

Example 58 was synthesised by reacting Compound F with Compound AD. The synthetic route for Compound AD is illustrated in Scheme 8.

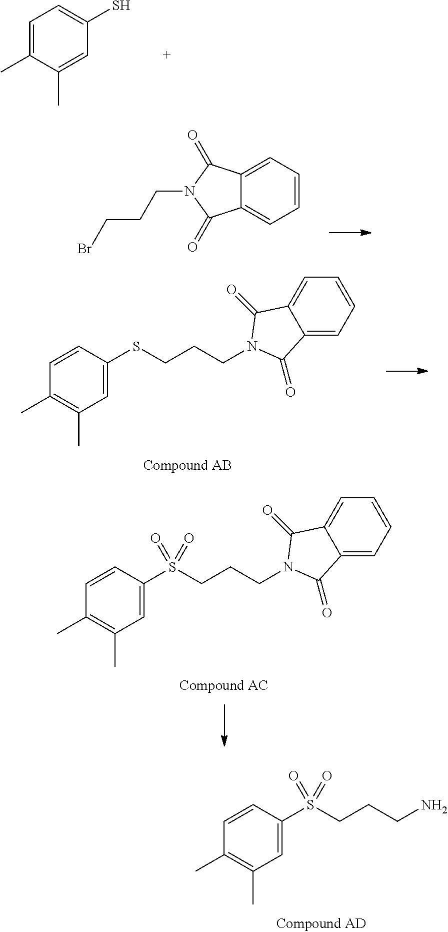

### 2-(3-[(3,4-dimethylphenyl)sulfanyl]propyl)-2,3-dihydro-1H-isoindole-1,3-dione—Compound AB

3,4-dimethylbenzene-1-thiol (1 g, 7.23 mmol) was dissolved in DMF (10 mL), 2-(3-bromopropyl)-1H-isoindole-1,3(2H)-dione (1.94 g, 7.23 mmol) was added followed by Cs₂CO₃ (4.71 g, 14.47 mmol). The reaction mixture was stirred at room temperature for 2 h. The Cs₂CO₃ was removed by filtration and the reaction mixture was diluted with EtOAc (25 mL) and washed with water (2×25 mL). The combined organics were dried by filtration through a hydrophobic frit and concentrated to yield the title compound (2.28 g, 92%)

¹H NMR (500 MHz, Chloroform-d) δ 7.83-7.70 (m, 2H), 7.70-7.57 (m, 2H), 7.11-7.06 (m, 1H), 7.08-7.01 (m, 1H), 6.99-6.92 (m, 1H), 3.74 (t, J=7.0 Hz, 2H), 2.86-2.77 (m, 2H), 2.14 (s, 6H), 1.95-1.84 (m, 2H). LCMS Method D: rt 1.56 min, 97%; m/z 325.95 (MH⁺)

### 2-[3-(3,4-dimethylbenzenesulfonyl)propyl]-2,3-dihydro-1H-isoindole-1,3-dione—Compound AC

To a solution of 2-{3-[(3,4-dimethylphenyl)sulfanyl]propyl}-2,3-dihydro-1H-isoindole-1,3-dione (750 mg, 2.3 mmol) in isopropanol (15 mL) was added Oxone (701 mg, 4.61 mmol) and water (7 mL). The reaction was stirred at room temperature for 54 h and over this time four further portions of Oxone (701 mg, 4.61 mmol) were added. The reaction was then quenched by careful addition of sodium thiosulphate, and stirred for 30 mins. The quenched reaction mixture was extracted with ethyl acetate, the organics washed with brine, dried over Na₂SO₄, and concentrated to yield an off white solid. The crude product was further purified by column chromatography (Biotage, 25 g SNAP KP-SIL, 50-100% DCM in heptane, 10 CV; then 0-100% EtOAc in heptane, 10 CV) to yield the title compound as a white solid (608 mg, 72%)

¹H NMR (500 MHz, DMSO-d6) δ 7.86-7.79 (m, 4H), 7.63 (s, 1H), 7.56 (d, J=7.5 Hz, 1H), 7.39 (d, J=8.2 Hz, 1H), 3.63 (t, J=7.0 Hz, 2H), 3.35 (t, J=8.0 Hz, 2H), 2.29 (d, J=2.8 Hz, 6H), 1.86 (quin, J=8.0, 6.7, 6.7 Hz, 2H). LCMS Method D: rt 1.30 min, 98%; m/z 358.0 (MH⁺)

### 3-(3,4-dimethylbenzenesulfonyl)propan-1-amine—Compound AD

2-[3-(3,4-dimethylbenzenesulfonyl)propyl]-2,3-dihydro-1H-isoindole-1,3-dione (608 mg, 1.7 mmol) was dissolved in Ethanol (13 mL). Hydrazine hydrate (1:1) (249 μl, 5.1 mmol) was added and the reaction stirred at 60° C. for 18 h. The reaction mixture was cooled to room temperature and concentrated. The residue was partitioned between 2M NaOH and ethyl acetate. The combined organics were dried over Na₂SO₄ and concentrated to give the title compound as a colourless oil (388 mg, 100%)

¹H NMR (500 MHz, Chloroform-d) δ 7.65 (s, 1H), 7.62 (dd, J=7.9, 1.6 Hz, 1H), 7.30 (d, J=7.9 Hz, 1H), 3.18-3.11 (m, 2H), 2.78 (t, J=6.8 Hz, 2H), 2.34 (s, 6H), 1.89-1.79 (m, 2H). LCMS Method A: rt 1.21 min, 94%; m/z 228.2 (MH⁺)

### 2-(4-tert-butylphenyl)-4-{[3-(3,4-dimethylbenzenesulfonyl)propyl]amino}-6H,7H-pyrazolo[1,5-a][1,3,5]triazin-7-one—Example 58

2-(4-tert-butylphenyl)-4-(methylsulfanyl)-6H,7H-pyrazolo[1,5-a][1,3,5]triazin-7-one (80 mg, 0.25 mmol) and 3-(3,4-dimethylbenzenesulfonyl)propan-1-amine (183 mg, 0.76 mmol) were dissolved in Pyridine (0.5 mL). The reaction mixture was heated to 40° C. for 96 h, then concentrated and purified using HPLC Method A, affording the title compound as a brown tacky solid (19.8 mg, 15%)

¹H NMR (500 MHz, Chloroform-d) δ 8.30 (d, J=8.6 Hz, 2H), 7.66 (s, 1H), 7.62 (d, J=8.3 Hz, 1H), 7.49 (d, J=8.6 Hz, 2H), 7.24 (d, J=7.5 Hz, 1H), 6.25 (t, J=5.9 Hz, 1H), 5.86 (s, 1H), 3.92 (m, 2H), 3.29 (t, J=7.3 Hz, 2H), 2.31-2.24 (m, 8H), 1.39 (s, 9H). LCMS Method B: rt 4.11 min, 97%; m/z 494.4 (MH⁺)

Example 59 was synthesised by reacting Compound F with Compound AF. The synthetic route for Compound AF is illustrated in Scheme 9.

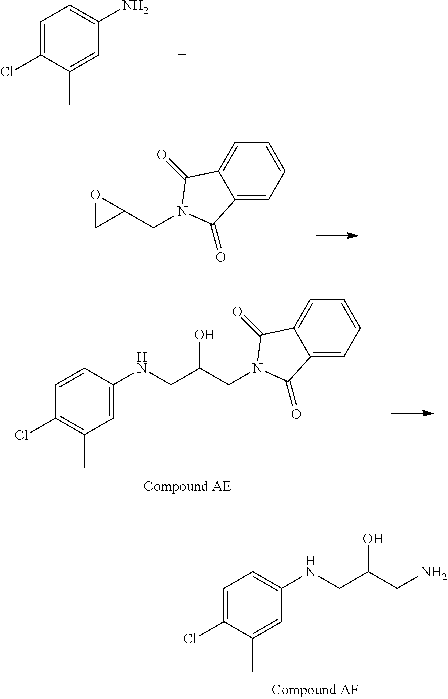

### 2-{3-[(4-chloro-3-methylphenyl)amino]-2-hydroxypropyl}-2,3-dihydro-1H-isoindole-1,3-dione—Compound AE

2-(oxiran-2-ylmethyl)-2,3-dihydro-1H-isoindole-1,3-dione (1 g, 4.92 mmol) and 4-chloro-3-methylaniline (697 mg, 4.92 mmol) were dissolved in Isopropanol (15 mL) and the reaction mixture was heated to 85° C. for 18 h. The reaction mixture was allowed to cool to room temperature and the solid formed was filtered off and washed with TBME, yielding the title compound as a yellow solid (1.2 g, 67%)

¹H NMR (500 MHz, Methanol-d4) δ 7.93-7.85 (m, 2H), 7.85-7.76 (m, 2H), 7.04 (d, J=8.6 Hz, 1H), 6.57 (d, J=2.7 Hz, 1H), 6.49-6.43 (m, 1H), 4.20-4.08 (m, 1H), 3.89-3.73 (m, 2H), 3.29-3.07 (m, 2H), 2.25 (s, 3H). LCMS Method A: rt 1.32 min, 82%; nm/z 344.95 (MH⁺)

### 1-amino-3-[(4-chloro-3-methylphenyl)amino]propan-2-ol—Compound AF

2-{3-[(4-chloro-3-methylphenyl)amino]-2-hydroxypropyl}-2,3-dihydro-1H-isoindole-1,3-dione (0.6 g, 1.74 mmol) was dissolved in Ethanol (10 mL) and hydrazine hydrate (1:1) (0.34 mL, 6.96 mmol) was added. The reaction mixture was stirred at 60° C. for 4 h. The reaction mixture was concentrated and the solid was washed with TBME (40 mL). The filtrate was collected and dried to give the title compound (219 mg, 56%)

¹H NMR (500 MHz, Methanol-d4) δ 7.02 (d, J=8.6 Hz, 1H), 6.56 (d, J=2.7 Hz, 1H), 6.48-6.41 (m, 1H), 3.79-3.69 (m, 1H), 3.18-3.09 (m, 1H), 3.07-2.97 (m, 1H), 2.80-2.74 (m, 1H), 2.66-2.58 (m, 1H), 2.24 (s, 3H). LCMS Method D: rt 0.79 min, 99%; m/z 215.0 (MH⁺)

### 2-(4-tert-butylphenyl)-4-({3-[(4-chloro-3-methylphenyl)amino]-2-hydroxypropyl}amino)-6H,7H-pyrazolo[1,5-a][1,3,5]triazin-7-one—Example 59

2-(4-tert-butylphenyl)-4-(methylsulfanyl)-6H,7H-pyrazolo[1,5-a][1,3,5]triazin-7-one (80 mg, 0.25 mmol) and 1-amino-3-[(4-chloro-3-methylphenyl)amino]propan-2-ol (164 mg, 0.76 mmol) were dissolved in Pyridine (0.5 mL). The reaction mixture was heated to 120° C. in a microwave for 2 h, then concentrated and purified using HPLC Method A, affording the title compound (5.3 mg, 4%)

¹H NMR (500 MHz, Methanol-d4) δ 8.22-8.14 (m, 2H), 7.48-7.38 (m, 2H), 6.98 (d, J=8.6 Hz, 1H), 6.53 (d, J=2.7 Hz, 1H), 6.50-6.40 (m, 1H), 5.67 (s, 1H), 4.57 (s, 1H), 4.25-4.11 (m, 1H), 4.05-3.92 (m, 1H), 3.71-3.62 (m, 1H), 3.29-3.15 (m, 2H), 2.17 (s, 3H), 1.36 (s, 9H). LCMS Method B: rt 4.24 min, 92%; m/z 481.2 (MH⁺)

Examples 60 and 61 were synthesized by reacting Compound F with amines (Compounds AG and AH) synthesized in a manner analogous to that used for Compound AF.

### 1-amino-3-(4-chloro-3-methylphenoxy)propan-2-ol—Compound AG

Compound AG was synthesized in two steps by reacting 4-chloro-3-methylphenol (512 μl, 4.92 mmol) with 2-(oxiran-2-ylmethyl)-2,3-dihydro-1H-isoindole-1,3-dione (1 g, 4.92 mmol) using caesium fluoride (45 μl, 1.23 mmol) in DMF (10 mL) in a reaction analogous to that for Compound AE, followed by deprotection with hydrazine hydrate (1:1) (0.34 mL, 6.96 mmol) in Ethanol (20 mL) in a reaction analogous to that for Compound AF, yielding the title compound (408 mg, 50% purity, 32% over two steps)

¹H NMR (500 MHz, Methanol-d4) δ 7.22 (d, J=8.8 Hz, 1H), 6.88 (d, J=2.9 Hz, 1H), 6.76 (dd, J=8.7, 3.0 Hz, 1H), 4.11-3.99 (m, 1H), 3.97-3.93 (m, 2H), 3.07-2.98 (m, 1H), 2.93-2.84 (m, 1H), 2.31 (s, 3H).

### 2-(4-tert-butylphenyl)-4-{[3-(4-chloro-3-methylphenoxy)-2-hydroxypropyl]amino}-6H,7H-pyrazolo[1,5-a][1,3,5]triazin-7-one—Example 60

2-(4-tert-butylphenyl)-4-(methylsulfanyl)-6H,7H-pyrazolo[1,5-a][1,3,5]triazin-7-one (60 mg, 0.19 mmol) and 1-amino-3-(4-chloro-3-methylphenoxy)propan-2-ol (164 mg, 50% purity, 0.38 mmol) were dissolved in Pyridine (0.5 mL). The reaction mixture was heated to 120° C. in a microwave for 2 h, then concentrated and purified using HPLC Method A, affording the title compound (24 mg, 25%)

¹H NMR (500 MHz, Chloroform-d) δ 8.20 (d, J=7.7 Hz, 2H), 7.42 (d, J=8.2 Hz, 2H), 7.17 (d, J=8.7 Hz, 1H), 6.89 (s, 1H), 6.77-6.71 (m, 1H), 6.70-6.59 (m, 1H), 5.84 (s, 1H), 4.35 (s, 1H), 4.13-3.91 (m, 3H), 3.77 (s, 1H), 2.27 (s, 3H), 1.32 (s, 9H). LCMS Method B: rt 4.40 min, 100%; m/z 482.3 (MH⁺)

### 1-amino-3-(cyclohexylamino)propan-2-ol—Compound AH

Compound AH was synthesized in two steps by reacting cyclohexanamine (0.73 g, 7.38 mmol) with 2-(oxiran-2-ylmethyl)-2,3-dihydro-1H-isoindole-1,3-dione (1.5 g, 7.38 mmol) in Isopropanol (25 mL) in a reaction analogous to that for Compound AE, followed by deprotection with hydrazine hydrate (1:1) (161 μl, 3.31 mmol) in Ethanol (5 mL) in a reaction analogous to that for Compound AF, yielding the title compound (142 mg, 50% purity, 25% over two steps)

¹H NMR (500 MHz, Methanol-d4) δ 3.81 (dt, J=8.1, 4.0 Hz, 1H), 2.97-2.80 (m, 2H), 2.80-2.57 (m, 3H), 1.98 (d, J=11.2 Hz, 2H), 1.79 (d, J=14.8 Hz, 2H), 1.73-1.56 (m, 1H), 1.39-1.25 (m, 2H), 1.25-1.10 (m, 3H).

### 2-(4-tert-butylphenyl)-4-{[3-(cyclohexylamino)-2-hydroxypropyl]amino}-6H,7H-pyrazolo[1,5-a][1,3,5]triazin-7-one—Example 61

2-(4-tert-butylphenyl)-4-(methylsulfanyl)-6H,7H-pyrazolo[1,5-a][1,3,5]triazin-7-one (160 mg, 0.51 mmol) and 1-amino-3-(cyclohexylamino)propan-2-ol (131 mg, 50% purity, 0.38 mmol) were dissolved in Pyridine (0.5 mL). The reaction mixture was heated to 120° C. in a microwave for 2 h, then concentrated and purified using HPLC Method A, affording the title compound (29 mg, 12%)

¹H NMR (500 MHz, Methanol-d4) δ 8.51 (s, 1H), 8.35-8.19 (m, 2H), 7.60-7.45 (m, 2H), 5.69 (s, 1H), 4.31-4.21 (m, 1H), 3.82 (d, J=5.7 Hz, 2H), 3.31 (s, 2H), 3.11-3.04 (m, 1H), 3.03-2.95 (m, 1H), 2.09-1.97 (m, 2H), 1.85-1.75 (m, 2H), 1.70-1.60 (m, 1H), 1.37 (s, 9H), 1.35-1.20 (m, 4H), 1.20-1.05 (m, 1H). LCMS Method B: rt 2.48 min, 99%; m/z 439.3 (MH⁺)

Example 62 was synthesised by reacting Compound F with Compound AK. The synthetic route for Compound AK is illustrated in Scheme 10.

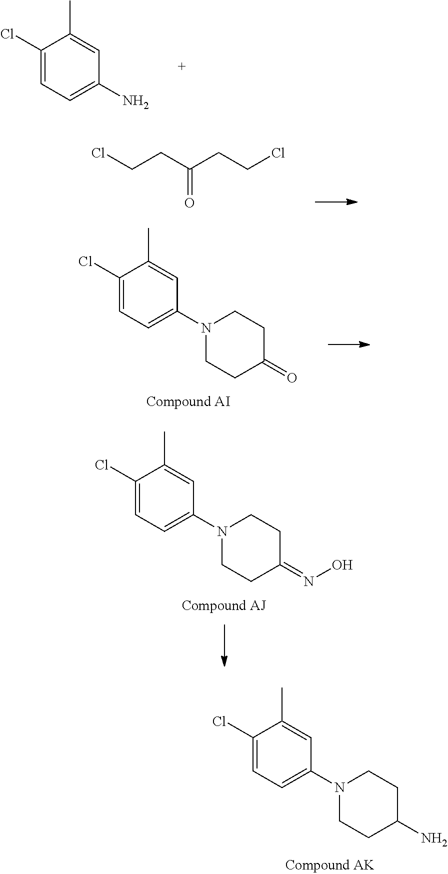

### 1-(4-chloro-3-methylphenyl)piperidin-4-one—Compound AI

To a refluxing slurry of disodium carbonate (0.9 g, 0.01 mol) in 10 mL MeOH was added simultaneously a solution of 1,5-dichloropentan-3-one (0.93 mL, 0.01 mol) in 5 mL MeOH and a solution of 4-chloro-3-methylaniline (1 g, 0.01 mol) in 5 mL MeOH dropwise over 10 mins. The reaction was allowed to reflux for a further 2.5 h, then allowed to cool and concentrated, poured into water (10 mL), and extracted with DCM (2×10 mL). The combined organics were dried over MgSO₄ and concentrated to yield 1.38 g free flowing orange/brown oil. The crude product was purified using column chromatography (Biotage, 50 g SNAP KP-SIL, 10-80% EtOAc in heptane, 10 CV) to yield the title compound as a light yellow free-flowing liquid (890 mg, 86% purity, 48%)

¹H NMR (500 MHz, Chloroform-d) 87.22 (d, J=8.7 Hz, 1H), 6.83 (d, J=2.9 Hz, 1H), 6.74 (dd, J=8.7, 3.0 Hz, 1H), 3.56 (t, J=6.1 Hz, 4H), 2.55 (t, J=6.1 Hz, 4H), 2.35 (s, 3H). LCMS Method D: rt 1.33 min, 91%; m/z 223.95 (MH⁺)

### N-[1-(4-chloro-3-methylphenyl)piperidin-4-ylidene]hydroxylamine—Compound AJ

1-(4-chloro-3-methylphenyl)piperidin-4-one (86% purity, 0.89 g, 3.42 mmol), hydroxylamine hydrochloride (1:1) (214 μl, 5.13 mmol) and dipotassium carbonate (0.85 g, 6.16 mmol) were stirred in 1:1 EtOH:water (4 mL) in a pressure vessel and the reaction was heated to 100° C. for 2 h. The reaction was poured into 10 mL water and the precipitate was filtered off and dried in a vacuum oven to yield the title compound as light orange crystals (692 mg, 83%)

¹H NMR (500 MHz, Chloroform-d) δ 7.30 (s, 1H), 7.20 (d, J=8.7 Hz, 1H), 6.79 (d, J=2.9 Hz, 1H), 6.70 (dd, J=8.7, 2.9 Hz, 1H), 3.38-3.34 (m, 2H), 3.31 (t, J=6.0 Hz, 2H), 2.75 (t, J=6.0 Hz, 2H), 2.49-2.44 (m, 2H), 2.33 (s, 3H). LCMS Method D: rt 1.28 min, 98%; m/z 239.0 (MH⁺)

### 1-(4-chloro-3-methylphenyl)piperidin-4-amine—Compound AK

A solution of N-[1-(4-chloro-3-methylphenyl)piperidin-4-ylidene]hydroxylamine (692 mg, 2.9 mmol) in toluene (3 mL) was added dropwise to a stirred solution of 3.5M sodium dihydrido[bis(2-methoxyethanolato-kappaO)]aluminate(1-) (4.14 mL, RED-Al) under nitrogen. The reaction was heated to reflux for 6 h. After cooling to room temperature the reaction mixture was quenched with aqueous ethanol (10 mL) and poured onto ice. The mixture was diluted to 20 mL with water and acidified to pH 1-2 with c.H₂SO₄ then filtered through a plug of celite and washed with toluene (2×10 mL). The aqueous layer was then basified to pH 8-9 using aq. KOH, diluted with 30 mL 10% Rochelle's salt and extracted with DCM (3×10 mL). The combined organics were dried over MgSO₄ and concentrated to yield 388 mg free flowing yellow oil, LCMS Method B indicated 52% title compound (rt 1.64), 38% imine intermediate (rt 1.71). Further purification of this material using HPLC method A afforded the pure title compound as a white solid (35.3 mg, 5%)

LCMS Method B: rt 1.58 min, 99%; m/z 225.2 (MH⁺)

### 2-(4-tert-butylphenyl)-4-{[1-(4-chloro-3-methylphenyl)piperidin-4-yl]amino}-6H,7H-pyrazolo[1,5-a][1,3,5]triazin-7-one—Example 62

2-(4-tert-butylphenyl)-4-(methylsulfanyl)-6H,7H-pyrazolo[1,5-a][1,3,5]triazin-7-one (40 mg, 0.13 mmol) and 1-(4-chloro-3-methylphenyl)piperidin-4-amine (35 mg, 0.15 mmol) were dissolved in 1,4-Dioxane (0.2 mL). The reaction mixture was heated to 130° C. in a microwave for 22 h, then concentrated and purified using HPLC Method A, affording the title compound (7.2 mg, 12%)

¹H NMR (500 MHz, Chloroform-d) δ 8.33 (d, J=8.5 Hz, 2H), 7.51 (d, J=8.5 Hz, 2H), 7.22 (d, J=9.0 Hz, 1H), 6.86 (d, J=3.5 Hz, 1H), 6.78 (dd, J=8.7, 2.7 Hz, 1H), 5.90 (d, J=7.4 Hz, 1H), 5.84 (s, 1H), 4.46-4.37 (m, 1H), 3.64 (dt, J=12.8, 3.7 Hz, 2H), 3.02 (t, J=10.9, 10.4 Hz, 2H), 2.36 (s, 3H), 2.30 (d, J=12.5 Hz, 2H), 1.88 (qd, J=10.6, 10.2, 9.3, 3.9 Hz, 2H), 1.37 (s, 9H). LCMS Method F: rt 5.44 min, 100%; m/z 491.2 (MH⁺)

Example 63 was synthesised by reacting Compound F with Compound AM. The synthetic route for Compound AM is illustrated in Scheme 11.

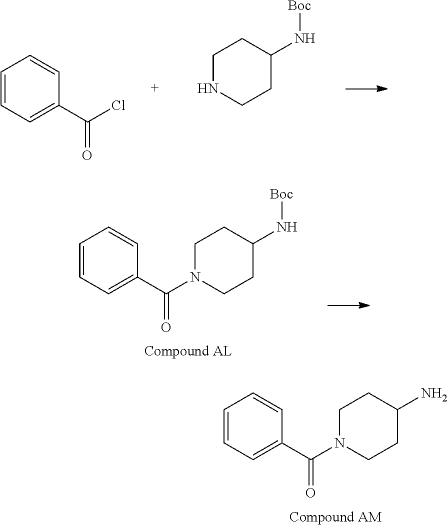 [SMILES: O=C(Cl)c1ccccc1 ; CC(C)(C)OC(=O)NC1CCNCC1 ; CC(C)(C)OC(=O)NC1CCN(C(=O)c2ccccc2)CC1 ; NC1CCN(C(=O)c2ccccc2)CC1]

### tert-butyl N-(1-benzoylpiperidin-4-yl)carbamate—Compound AL

tert-butyl N-(piperidin-4-yl)carbamate (330 mg, 1.65 mmol) and Triethylamine (250 μl, 1.8 mmol) were dissolved in DCM (4 mL) under nitrogen and the mixture was cooled to 0° C. Benzoyl chloride (211 mg, 1.5 mmol) was then added dropwise, and the mixture was allowed to warm to room temperature and stirred for 3 h. The reaction was washed with water, followed by brine, and the organic layer was separated using a phase separator and concentrated to yield the title compound as a white solid (464 mg, 96%).

¹H NMR (500 MHz, Chloroform-d) δ 7.77 (dd, J=8.3, 1.3 Hz, 2H), 7.56-7.50 (m, 1H), 7.48-7.44 (m, 2H), 5.97 (d, J=7.4 Hz, 1H), 4.22-4.02 (m, 1H), 2.95 (t, J=12.0 Hz, 2H), 2.10-2.02 (m, 2H), 1.49 (s, 9H), 1.49-1.47 (m, 2H), 1.46-1.38 (m, 2H). LCMS Method D: rt 1.08 min, 94%; m/z 248.9 (MH⁺-^{t}Bu)

### 1-benzoylpiperidin-4-amine—Compound AM

tert-butyl N-(1-benzoylpiperidin-4-yl)carbamate (464 mg, 1.52 mmol) was stirred in TFA (3 mL) under nitrogen at room temperature for 2 h. The reaction mixture was then concentrated and purified using an SCX-II column, using methanol to elute the impurities and 0.7M NH3 in methanol to elute the product. The product fractions were combined and concentrated to yield the title compound as a colourless oil (204 mg, 66%)

¹H NMR (500 MHz, Chloroform-d) δ 7.79-7.76 (m, 2H), 7.54-7.50 (m, 1H), 7.48-7.43 (m, 2H), 6.06-6.01 (m, 1H), 4.16-4.06 (m, 1H), 3.13 (dt, J=12.6, 3.4 Hz, 2H), 2.78 (td, J=12.3, 2.5 Hz, 2H), 2.10-2.04 (m, 2H), 1.44 (qd, J=11.4, 4.0 Hz, 2H).

### 4-[(1-benzoylpiperidin-4-yl)amino]-2-(4-tert-butylphenyl)-6H,7H-pyrazolo[1,5-a][1,3,5]triazin-7-one—Example 63

2-(4-tert-butylphenyl)-4-(methylsulfanyl)-6H,7H-pyrazolo[1,5-a][1,3,5]triazin-7-one (70 mg, 0.22 mmol) and 1-benzoylpiperidin-4-amine (102 mg, 0.47 mmol) were dissolved in Pyridine (0.5 mL). The reaction mixture was heated to 80° C. for 18 h, then concentrated and purified using HPLC Method A, affording the title compound (30.6 mg, 29%)

¹H NMR (500 MHz, Chloroform-d) δ 8.27 (d, J=8.6 Hz, 2H), 7.77-7.73 (m, 2H), 7.51-7.45 (m, 3H), 7.39 (t, J=7.7 Hz, 2H), 6.36 (d, J=8.0 Hz, 1H), 5.86 (s, 1H), 5.33 (d, J=13.2 Hz, 2H), 4.40-4.29 (m, 1H), 3.29 (t, J=11.9 Hz, 2H), 2.16 (d, J=10.6 Hz, 2H), 1.66 (qd, J=12.6, 3.8 Hz, 2H), 1.34 (s, 9H). LCMS Method B: rt 3.97 min, 98%; m/z 471.3 (MH⁺)

Example 64 was synthesised by reacting Compound F with Compound AO. The synthetic route for Compound AO is illustrated in Scheme 12.

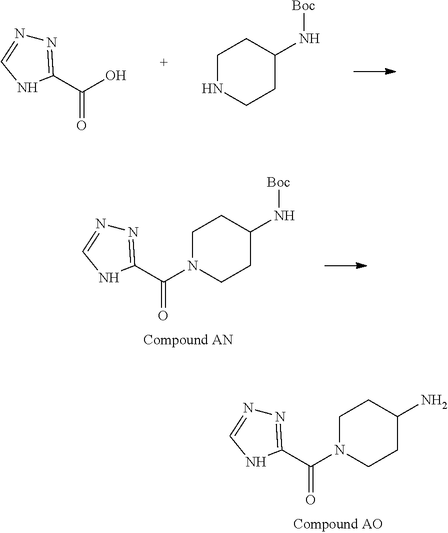 [SMILES: O=C(O)c1nnc[nH]1 ; CC(C)(C)OC(=O)NC1CCNCC1 ; CC(C)(C)OC(=O)NC1CCN(C(=O)c2nnc[nH]2)CC1 ; NC1CCN(C(=O)c2nnc[nH]2)CC1]

### tert-butyl N-[1-(4H-1,2,4-triazole-3-carbonyl)piperidin-4-yl]carbamate—Compound AN

4H-1,2,4-triazole-3-carboxylic acid (186 mg, 1.65 mmol), HATU (683 mg, 1.8 mmol) and DIPEA (387 mg, 3 mmol) were dissolved in DMF (5 mL) under nitrogen. After 10 mins of stirring tert-butyl piperidin-4-ylcarbamate (300 mg, 1.5 mmol) was added. The reaction was stirred at room temperature for 18 h, then concentrated and acidified to pH 4. The mixture was extracted with ethyl acetate and the combined organics were dried over Na₂SO₄ and concentrated to yield the title compound as a white solid (482 mg, 87% purity, 95%)

LCMS Method D: rt 1.02 min, 87%; m/z 240.0 (MH⁺-^{t}Bu)

### 1-(4H-1,2,4-triazole-3-carbonyl)piperidin-4-amine—Compound AO

tert-butyl N-[1-(4H-1,2,4-triazole-3-carbonyl)piperidin-4-yl]carbamate (87% purity, 482 mg, 1.42 mmol) was stirred in TFA (2 mL) at room temperature for 3 h. The reaction mixture was then concentrated, and purified using an SCX-II column, using methanol to elute the impurities and 0.7M NH₃ in methanol to elute the product. The basic eluents were concentrated to yield the title compound as a colourless oil (228 mg, 89%)

LCMS Method D: rt 0.19 min; m/z 195.95 (MH⁺)

### 2-(4-tert-butylphenyl)-4-{[11-(4H-1,2,4-triazole-3-carbonyl)piperidin-4-yl]amino}-6H,7H-pyrazolo[1,5-a][1,3,5]triazin-7-one—Example 64

2-(4-tert-butylphenyl)-4-(methylsulfanyl)-6H,7H-pyrazolo[1,5-a][1,3,5]triazin-7-one (80 mg, 0.25 mmol) and 1-(4H-1,2,4-triazole-3-carbonyl)piperidin-4-amine (114 mg, 0.55 mmol) were dissolved in Pyridine (0.5 mL). The reaction mixture was heated to 130° C. in a microwave for 6 h, then concentrated and purified using HPLC Method A, affording the title compound as an orange tacky solid (6.1 mg, 5%)

¹H NMR (500 MHz, Chloroform-d) δ 8.26 (d, J=8.5 Hz, 2H), 7.52 (d, J=8.6 Hz, 2H), 5.74 (s, 1H), 5.47-5.38 (m, 2H), 4.25-4.18 (m, 1H), 1.96 (d, J=11.0 Hz, 2H), 1.86-1.77 (m, 2H), 1.33 (s, 9H). LCMS Method B: rt 3.11 min, 100%; m/z 462.3 (MH⁺)

Example 65 was synthesised by reacting Compound F with Compound AR. The synthetic route for Compound AR is illustrated in Scheme 13.

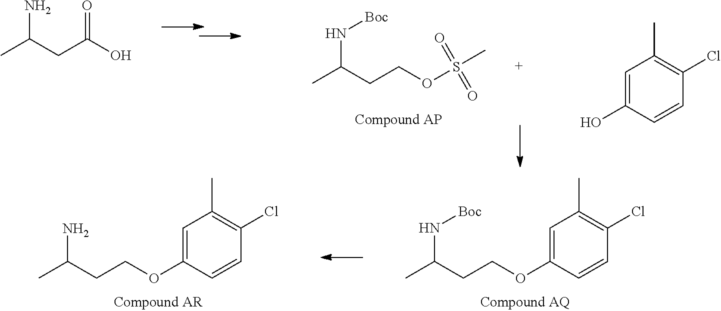

### tert-butyl N-[4-(methanesulfonyloxy)butan-2-yl]carbamate—Compound AP

2.4M lithium aluminium hydride in THF (3 mL, 7.27 mmol) was added dropwise to a stirred suspension of 3-aminobutanoic acid (500 mg, 4.85 mmol) in THF (5 mL) cooled to 0° C. The reaction was then heated to reflux for 18 h, after which time it was cooled to 0° C., and quenched by the careful sequential addition of water (0.4 mL), 15% aqueous NaOH (0.4 mL) and water (0.4 mL). The mixture was stirred for 30 minutes and then filtered through Celite and Na₂SO₄, washing the filter pad with THF. Concentration of the filtrate afforded a colourless oil (846 mg), which was dissolved in ethyl acetate (4 mL) and to it added a solution of di-tert-butyl dicarbonate (1.1 mL, 4.84 mmol) in ethyl acetate (3 mL) over 1 h. The reaction mixture was stirred for further 1 h, then washed with water, followed by brine, dried over Na₂SO₄, and concentrated to yield a pale orange oil (491 mg). 100 mg of this material was dissolved in DCM (1 mL) and triethylamine (147 μl, 1.06 mmol) was added. The mixture was stirred under nitrogen at 0° C. and methanesulfonyl chloride (61 mg, 0.53 mmol) was added dropwise. The reaction was stirred at room temperature for 18 h, then diluted with DCM and washed with water followed by brine, dried over Na₂SO₄, and concentrated to yield the title compound as a yellow oil. (150.6 mg)

¹H NMR (500 MHz, Chloroform-d) δ 4.28 (t, J=6.3, 5.7 Hz, 2H), 3.83 (m, 1H) 3.03 (s, 3H), 2.92-1.83 (m, 2H), 1.44 (s, 9H), 1.20 (d, J=5.7 Hz, 3H)

### tert-butyl N-[4-(4-chloro-3-methylphenoxy)butan-2-yl]carbamate—Compound AQ

tert-butyl N-[4-(methanesulfonyloxy)butan-2-yl]carbamate (336 mg, 1.26 mmol), K₂CO₃ (347 mg, 2.51 mmol), KI (208 mg, 1.26 mmol) and 4-chloro-3-methylphenol (157 μl, 1.51 mmol) were dissolved in DMF (2 mL). The reaction was stirred at 80° C. for 4 h, then at room temperature for 18 h. The mixture was the partitioned between ethyl acetate and water, the organic layer was retained and washed a further 2 times with water, followed by brine. The organic layer was dried over Na₂SO₄ and concentrated to yield the title compound as a yellow oil containing approx. 40% 4-chloro-3-methylphenol by mass (359 mg, 60% purity, 55%)

LCMS Method D: rt 1.59 min; m/z 336.1 (MNa⁺)

### 4-(3-aminobutoxy)-1-chloro-2-methylbenzene—Compound AR

tert-butyl N-[4-(4-chloro-3-methylphenoxy)butan-2-yl]carbamate (60%, 515 mg, 0.98 mmol) was stirred in TFA (2 mL, 26.12 mmol) under nitrogen at room temperature for 1 h. The reaction mixture was then concentrated, and purified using an SCX-II column, using methanol to elute the impurities and 0.7M NH₃ in methanol to elute the product. The basic eluents were concentrated to yield the title compound as a yellow oil (139.3 mg, 65%)

¹H NMR (500 MHz, Chloroform-d) δ 7.22 (d, J=8.6 Hz, 1H), 6.79 (d, J=3.0 Hz, 1H), 6.71 (dd, J=9.3, 2.6 Hz, 1H), 4.10-3.99 (m, 2H), 3.20 (h, J=6.7, 6.3, 5.4 Hz, 1H), 2.35 (s, 3H), 1.89-1.72 (m, 2H), 1.17 (d, J=6.3 Hz, 3H). LCMS Method D: rt 0.87 min, 98%; m/z 214.0 (MH⁺)

### 2-(4-tert-butylphenyl)-4-{[4-(4-chloro-3-methylphenoxy)butan-2-yl]amino}-6H,7H-pyrazolo[1,5-a][1,3,5]triazin-7-one—Example 65

2-(4-tert-butylphenyl)-4-(methylsulfanyl)-6H,7H-pyrazolo[1,5-a][1,3,5]triazin-7-one (80 mg, 0.25 mmol) and 4-(3-aminobutoxy)-1-chloro-2-methylbenzene (139 mg, 0.62 mmol) were dissolved in Dioxane (0.5 mL). The reaction mixture was heated to 130° C. in a microwave for 12 h, then concentrated and purified using HPLC Method A, affording the title compound as an orange tacky solid (20 mg, 16%)

¹H NMR (500 MHz, Chloroform-d) δ 8.31 (d, J=8.7 Hz, 2H), 7.45 (d, J=8.7 Hz, 2H), 7.15 (d, J=8.0 Hz, 1H), 6.75 (d, J=2.9 Hz, 1H), 6.64 (dd, J=8.7, 2.7 Hz, 1H), 6.13 (d, J=8.6 Hz, 1H), 5.81 (s, 1H), 4.75 (quin, J=7.7, 7.3, 6.1 Hz, 1H), 4.20-4.12 (m, 1H), 4.13-4.09 (m, 1H), 2.24 (s, 3H), 2.20 (quin, J=6.4, 5.7, 5.2 Hz, 2H), 1.50 (d, J=6.6 Hz, 3H), 1.36 (s, 9H). LCMS Method E: rt 4.82 min, 98%; m/z 480.15 (MH⁺)

Example 66 was synthesised by reacting Compound F with Compound AT. The synthetic route for Compound AT is illustrated in Scheme 14.

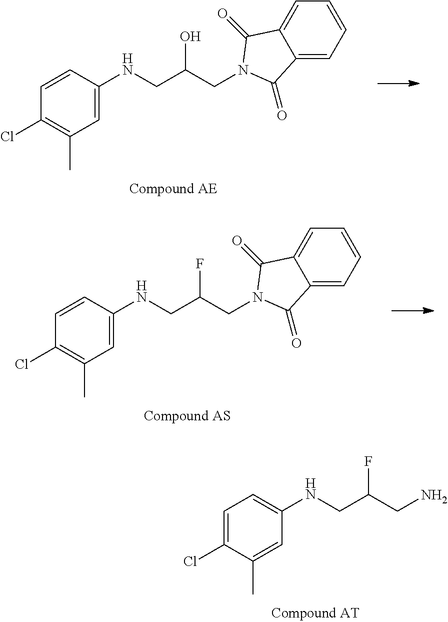 [SMILES: Cc1cc(NCC(O)CN2C(=O)c3ccccc3C2=O)ccc1Cl ; Cc1cc(NCC(F)CN2C(=O)c3ccccc3C2=O)ccc1Cl ; Cc1cc(NCC(F)CN)ccc1Cl]

### 2-{3-[(4-chloro-3-methylphenyl)amino]-2-fluoropropyl}-2,3-dihydro-1H-isoindole-1,3-dione—Compound AS

2-{3-[(4-chloro-3-methylphenyl)amino]-2-hydroxypropyl}-2,3-dihydro-1H-isoindole-1,3-dione (500 mg, 1.45 mmol) was dissolved in DCM (25 mL) and the solution was cooled to −78° C. under nitrogen. 2.7M Deoxofluor in toluene (1.61 mL, 4.35 mmol) was added dropwise. The reaction mixture was stirred at −78° C. for 1 h and then at room temperature for 120 h, then poured over ice and quenched with K₂CO₃. The aqueous layer was washed with DCM (3×25 mL). The combined organics were washed with brine (10 mL), dried over Na₂SO₄ and concentrated. The crude product was purified by column chromatography (Biotage, 25 g SNAP KP-SIL, 0-50% EtOAc in Heptane, 10 CV) yielding the title compound (100 mg, 67% purity, 11.9%)

LCMS Method C: rt 1.27 min, 67%; m/z 346.85 (MH⁺)

### N-(3-amino-2-fluoropropyl)-4-chloro-3-methylaniline—Compound AT

2-{3-[(4-chloro-3-methylphenyl)amino]-2-fluoropropyl}-2,3-dihydro-1H-isoindole-1,3-dione (67%, 100 mg, 0.19 mmol) was dissolved in Ethanol (5 mL) and hydrazine hydrate (1:1) (34 μl, 0.69 mmol) was added. The reaction mixture was stirred at 60° C. for 6 h, then concentrated and used without purification.

LCMS Method C: rt 0.82 min, m/z 216.9 (MH⁺)

### 2-(4-tert-butylphenyl)-4-({3-[(4-chloro-3-methylphenyl)amino]-2-fluoropropyl}amino)-6H,7H-pyrazolo[1,5-a][1,3,5]triazin-7-one—Example 66

2-(4-tert-butylphenyl)-4-(methylsulfanyl)-6H,7H-pyrazolo[1,5-a][1,3,5]triazin-7-one (60 mg, 0.19 mmol) and N-(3-amino-2-fluoropropyl)-4-chloro-3-methylaniline (38 mg, 0.17 mmol) were dissolved in Pyridine (0.5 mL). The reaction mixture was heated to 120° C. in a microwave for 6 h, then concentrated and purified using HPLC Method A, affording the title compound (6.6 mg, 7%)

¹H NMR (500 MHz, Methanol-d4) δ 8.30-8.02 (m, 2H), 7.59-7.28 (m, 2H), 6.97 (d, J=8.3 Hz, 1H), 6.59-6.50 (m, 2H), 5.64 (s, 1H), 4.75-4.45 (m, 3H), 4.14-3.98 (m, 1H), 3.98-3.86 (m, 2H), 2.11 (s, 3H), 1.37 (s, 9H). LCMS Method B: rt 4.62 min, 100%; m/z 483.2 (MH⁺)

Example 67 was synthesised by reacting Compound AW with Compound E. The synthetic route for Compound AW and subsequent reaction with Compound E are illustrated in Scheme 15.

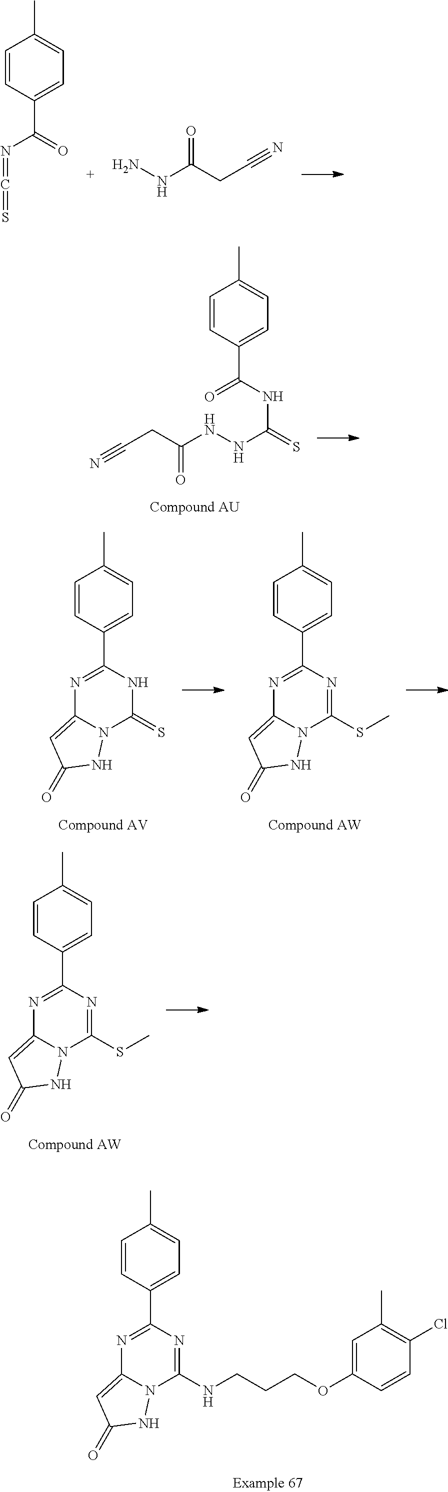

### 2-cyano-N-({[(4-methylphenyl)formamido]methanethioyl}amino)acetamide—Compound AU

2-cyanoacetohydrazide (2.86 g, 28.82 mmol) was dissolved in Acetone (40 mL) and 1-isothiocyanato-4-methylbenzene (4.3 g, 0.03 mol) was added portionwise. The reaction mixture was heated to reflux for 45 mins, then concentrated and the solid residue triturated with water, filtered off and dried under vacuum to afford the title compound as an off-white solid (7.5 g, 78% purity, 75%)

LCMS Method D: rt 1.07 min, 78%; m/z 276.95 (MH⁺)

### 2-(4-methylphenyl)-4-sulfanylidene-3H,4H,6H,7H-pyrazolo[1,5-a][1,3,5]triazin-7-one—Compound AV

2-cyano-N-({[(4-methylphenyl)formamido]methanethioyl}amino)acetamide (78%, 7.5 g, 21.17 mmol) was dissolved in 5% KOH in water (50 mL) and the reaction mixture was heated to reflux for 30 mins, then acidified to pH 1 with 2M aq. HCl. The precipitate formed was collected and dried under vacuum, and triturated with heptane to yield the title compound as a yellow powder (6.68 g, 80% purity, 97%)

¹H NMR (500 MHz, DMSO-d6) 13.56 (s, 1H), 11.77 (s, 1H), 8.02 (d, J=8.3 Hz, 2H), 7.37 (d, J=8.1 Hz, 2H), 6.01 (s, 1H), 2.41 (s, 3H). LCMS Method D: rt 1.04 min, 94%; m/z 258.9 (MH⁺)

### 2-(4-methylphenyl)-4-(methylsulfanyl)-6H,7H-pyrazolo[1,5-a][1,3,5]triazin-7-one—Compound AW

2-(4-methylphenyl)-4-sulfanylidene-3H,4H,6H,7H-pyrazolo[1,5-a][1,3,5]triazin-7-one (2 g, 7.74 mmol) and K₂CO₃ (2.14 g, 15.49 mmol) were dissolved in Acetone (20 mL) and heated to 65° C. Iodomethane (386 μl, 6.19 mmol) in Acetone (10 mL) was added dropwise and the reaction mixture was stirred at 65° C. for 3 h, then concentrated and triturated with 30 mL MeOH. Column chromatography using Biotage FCC (50 g SNAP KP-SiO2, 0-15% MeOH in DCM, 10 CV) yielded the title compound as a yellow solid (1.64 g, 30% purity, 30%)

LCMS Method D: rt 1.29 min, 30%; m/z 272.95 (MH⁺)

### 4-{[3-(4-chloro-3-methylphenoxy)propyl]amino}-2-(4-methylphenyl)-6H,7H-pyrazolo[1,5-a][1,3,5]triazin-7-one—Example 67

2-(4-methylphenyl)-4-(methylsulfanyl)-6H,7H-pyrazolo[1,5-a][1,3,5]triazin-7-one (50 mg, 30% purity, 0.06 mmol) and 4-(3-aminopropoxy)-1-chloro-2-methylbenzene (110 mg, 0.55 mmol) were dissolved in Acetonitrile (2 mL) and DMF (0.5 mL). The reaction mixture was heated to 130° C. in a microwave for 6 h, then concentrated and purified using Biotage FCC (10 g SNAP KP-SiO₂, 0-100% EtOAc in Heptane), affording the title compound (5 mg, 22%)

¹H NMR (500 MHz, Chloroform-d) δ 8.29 (d, J=6.5 Hz, 2H), 7.18 (d, J=8.7 Hz, 1H), 6.78 (s, 1H), 6.69 (m, 1H), 6.34 (s, 1H), 5.78 (s, 1H), 4.11 (s, 2H), 3.96 (s, 2H), 2.42 (s, 3H), 2.32-2.17 (m, 5H). N.b. signal for 2H in aromatic region obscured by CHCl₃ signal at 7.26 ppm. LCMS Method B: rt 4.36 min, 90%; m/z 424.1 (MH⁺)

Example 68 was synthesized by reacting Compound AW with Compound O.

### 4-({3-[(4-chloro-3-methylphenyl)amino]propyl}amino)-2-(4-methylphenyl)-6H,7H-pyrazolo[1,5-a][1,3,5]triazin-7-one—Example 68

2-(4-methylphenyl)-4-(methylsulfanyl)-6H,7H-pyrazolo[1,5-a][1,3,5]triazin-7-one (100 mg, 30% purity, 0.12 mmol) and N-(3-aminopropyl)-4-chloro-3-methylaniline (192 mg, 0.97 mmol) were dissolved in 1,4-Dioxane (4 mL). The reaction mixture was heated to 130° C. in a microwave for 6 h, then concentrated and purified using HPLC Method A, affording the title compound (16.6 mg, 36%)

¹H NMR (500 MHz, Methanol-d4) δ 8.30-8.05 (m, 2H), 7.25 (d, J=8.1 Hz, 2H), 6.98 (d, J=8.6 Hz, 1H), 6.49 (d, J=2.9 Hz, 1H), 6.43-6.38 (m, 1H), 5.63 (s, 1H), 3.83 (t, J=6.8 Hz, 2H), 3.23 (t, J=6.6 Hz, 2H), 2.41 (s, 3H), 2.18 (s, 3H), 2.11-1.97 (m, 2H). LCMS Method B: rt 4.06 min, 100%; m/z 423.2 (MH⁺)

Examples 69 and 70 were synthesised by reacting Compound AW with commercially available amines.

### 4-[(1-benzylpiperidin-4-yl)amino]-2-(4-methylphenyl)-6H,7H-pyrazolo[1,5-a][1,3,5]triazin-7-one—Example 69

2-(4-methylphenyl)-4-(methylsulfanyl)-6H,7H-pyrazolo[1,5-a][1,3,5]triazin-7-one (80 mg, 30% purity, 0.10 mmol) and 1-benzylpiperidin-4-amine (168 mg, 0.88 mmol) were dissolved in 1,4-Dioxane (1 mL). The reaction mixture was heated to 130° C. in a microwave for 3 h, then concentrated and purified using HPLC Method A, affording the title compound as a salt with formic acid (12.8 mg, 35%)

¹H NMR (500 MHz, Chloroform-d) δ 8.35 (s, 1H), 8.15 (d, J=8.1 Hz, 2H), 7.32 (m, 5H), 7.17 (d, J=8.1 Hz, 2H), 5.69 (s, 1H), 4.26 (s, 1H), 3.82 (s, 2H), 3.17 (d, J=9.9 Hz, 2H), 2.49 (t, J=11.0 Hz, 2H), 2.34 (s, 3H), 1.95 (m, 2H). N.b. 2H obscured by water signal LCMS Method B: rt 2.23 min, 93%; m/z 415.3 (MH⁺)

### 4-(butylamino)-2-(4-methylphenyl)-6H,7H-pyrazolo[1,5-a][1,3,5]triazin-7-one—Example 70

2-(4-methylphenyl)-4-(methylsulfanyl)-6H,7H-pyrazolo[1,5-a][1,3,5]triazin-7-one (100 mg, 30% purity, 0.12 mmol) was dissolved in butan-1-amine (500 μl, 5.06 mmol).

The reaction was stirred at room temperature for 30 mins, then allowed to stand for 4 days. The mixture was concentrated, and purified using HPLC Method A, affording the title compound as a white powder (19.5 mg, 59%)

¹H NMR (500 MHz, DMSO-d6) δ 10.90 (s, 1H), 8.24 (d, J=8.2 Hz, 2H), 8.20 (t, J=5.9 Hz, 1H), 7.30 (d, J=8.1 Hz, 2H), 5.67 (s, 1H), 3.60 (q, J=6.8 Hz, 2H), 2.37 (s, 3H), 1.67 (quin, J=7.3 Hz, 2H), 1.38 (h, J=7.3 Hz, 2H), 0.94 (t, J=7.4 Hz, 3H). LCMS Method B: rt 3.68 min, 84%; n/z 298.1 (MH⁺)

Example 71 was synthesised from Compound F and Compound E according the route illustrated in Scheme 16.

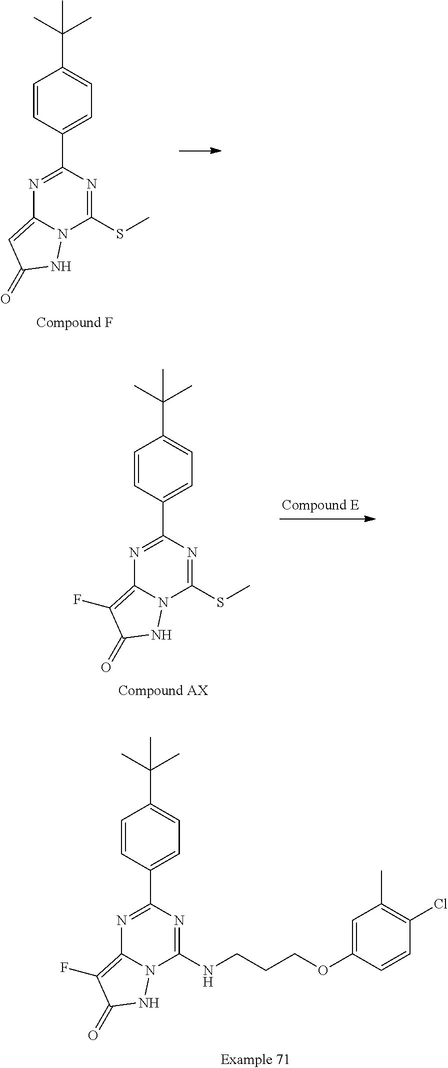 [SMILES: CSc1nc(-c2ccc(C(C)(C)C)cc2)nc2cc(=O)[nH]n12 ; CSc1nc(-c2ccc(C(C)(C)C)cc2)nc2c(F)c(=O)[nH]n12 ; Cc1cc(OCCCNc2nc(-c3ccc(C(C)(C)C)cc3)nc3c(F)c(=O)[nH]n23)ccc1Cl]

### 2-(4-tert-butylphenyl)-8-fluoro-4-(methylsulfanyl)-6H,7H-pyrazolo[1,5-a][1,3,5]triazin-7-one—Compound AX

To a solution of 2-(4-tert-butylphenyl)-4-(methylsulfanyl)-6H,7H-pyrazolo[1,5-a][1,3,5]triazin-7-one (150 mg, 0.25 mmol) in DMF (3 mL) was added 1-(chloromethyl)-4-fluoro-1,4-diazoniabicyclo[2.2.2]octane ditetrafluoroborate (169 mg, 0.48 mmol) at room temperature under nitrogen. After 1 h the reaction was quenched with sat. aq. NH₄Cl and extracted with EtOAc. The organic layer was washed with water, dried over MgSO₄, and concentrated. The crude product was purified using Biotage FCC (10 g SNAP KP-SiO2, 20-100% EtOAc in Heptane), affording the title compound as an orange solid (17 mg, 60% purity, 6%)

¹H NMR (500 MHz, Chloroform-d) δ 8.37 (d, J=8.5 Hz, 2H), 7.45 (d, J=8.6 Hz, 2H), 2.78 (s, 3H), 1.98 (s, 9H)

### 2-(4-tert-butylphenyl)-4-{[3-(4-chloro-3-methylphenoxy)propyl]amino}-8-fluoro-6H,7H-pyrazolo[1,5-a][1,3,5]triazin-7-one—Example 71

2-(4-tert-butylphenyl)-8-fluoro-4-(methylsulfanyl)-6H,7H-pyrazolo[1,5-a][1,3,5]triazin-7-one (17 mg, 60% purity, 0.03 mmol) and 4-(3-aminopropoxy)-1-chloro-2-methylbenzene (12 mg, 0.06 mmol) were stirred in Pyridine at 80° C. for 19 h, then concentrated and purified using HPLC Method A, affording the title compound as a white solid (2.5 mg, 16%)

¹H NMR (250 MHz, Methanol-d4) δ 8.31-8.19 (m, 2H), 7.52-7.36 (m, 2H), 7.15 (d, J=8.7 Hz, 1H), 6.85-6.63 (m, 2H), 4.14 (t, J=5.7 Hz, 2H), 3.94 (t, J=6.4 Hz, 2H), 2.34-2.14 (m, 5H), 1.38 (s, 9H). LCMS Method E: rt 5.02 min, 100%; m/z 484.05 (MH⁺)

Example 72 was synthesised from Example 37 as illustrated in Scheme 17.

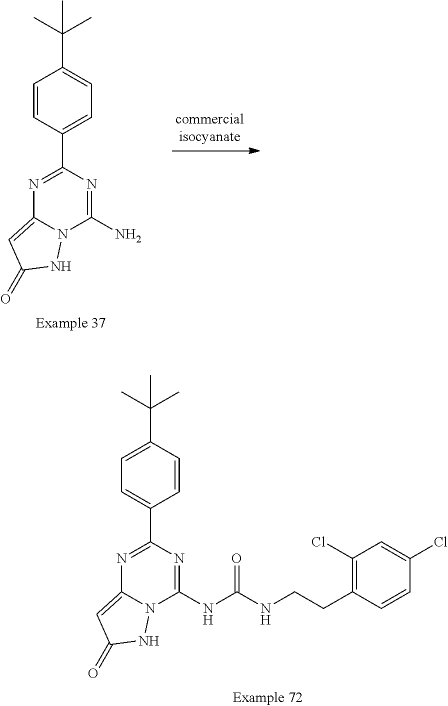 [SMILES: CC(C)(C)c1ccc(-c2nc(N)n3[nH]c(=O)cc3n2)cc1 ; CC(C)(C)c1ccc(-c2nc(NC(=O)NCCc3ccc(Cl)cc3Cl)n3[nH]c(=O)cc3n2)cc1]

### 4-amino-2-(4-tert-butylphenyl)-6H,7H-pyrazolo[1,5-a][1,3,5]triazin-7-one—Example 72

4-amino-2-(4-tert-butylphenyl)-6H,7H-pyrazolo[1,5-a][1,3,5]triazin-7-one (45 mg, 0.16 mmol) was dissolved in Pyridine (1 mL). DIPEA (110 μl, 0.64 mmol) was added followed by 2,4-dichloro-1-(2-isocyanatoethyl)benzene (50 mg, 0.23 mmol). The reaction mixture was stirred at room temperature for 24 h, then at 50° C. for 2 h. p-methoxybenzyl chloride (38 μl, 0.27 mmol) was added and the reaction mixture was stirred at room temperature for 18 h. The mixture was concentrated, and purified using HPLC Method A, affording the title compound as a white powder (9.1 mg, 10%)

¹H NMR (500 MHz, Chloroform-d) δ 9.53 (t, J=5.9 Hz, 1H), 8.76 (s, 1H), 7.84 (d, J=8.5 Hz, 2H), 7.44-7.35 (m, 3H), 7.21 (d, J=8.2 Hz, 1H), 7.18-7.06 (m, 1H), 5.95 (s, 1H), 3.82-3.71 (m, 2H), 3.11 (t, J=6.7 Hz, 2H), 1.37 (s, 9H). LCMS Method B: rt 4.80 min, 90%; m/z 499.2 (MH⁺)

Examples 73 to 84 were synthesized by reacting Compound F with commercially available amines as illustrated in Scheme 18.

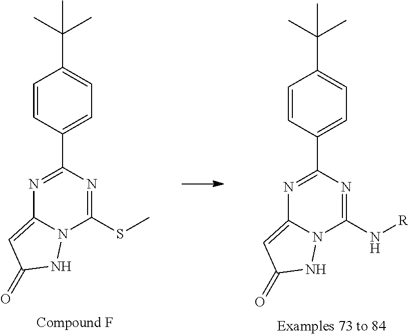

### 2-(4-tert-butylphenyl)-4-(hexadecylamino)-6H,7H-pyrazolo[1,5-a][1,3,5]triazin-7-one—Example 73

2-(4-tert-butylphenyl)-4-(methylsulfanyl)-6H,7H-pyrazolo[1,5-a][1,3,5]triazin-7-one (75 mg, 0.239 mmol) and hexadecane-1-amine (288 mg, 1.19 mmol) were dissolved in Pyridine (1 mL). The reaction mixture was heated in a microwave at 140° C. for 3 h, then evaporated to dryness and filtered by FCC (Biotage, 10 g SNAP KP-SIL, 25% to 75% EtOAc in heptane, 10 CV) to give a crude product that was purified by FCC (Biotage, 10 g SNAP KP-SIL, 0 to 35% EtOAc in heptane, 10 CV) to give the title product as a pale yellow solid (6 mg, 5%).

¹H NMR (500 MHz, Chloroform-d) δ 8.35 (d, J=8.6 Hz, 2H), 7.52-7.46 (m, 2H), 5.91 (t, J=5.7 Hz, 1H), 5.83 (s, 1H), 3.75 (q, J=6.9 Hz, 2H), 1.77 (p, J=7.3 Hz, 2H), 1.47 (dt, J=14.8, 6.9 Hz, 2H), 1.36 (s, 9H), 1.25 (brs, 24H), 0.88-0.85 (m, 3H). LCMS method H: rt 2.35 min, 77%; m/z 508.5 (MH⁺)

### 4-{[3-(benzylamino)propyl]amino}-2-(4-tert-butylphenyl)-6H,7H-pyrazolo[1,5-a][1,3,5]triazin-7-one—Example 74

2-(4-tert-butylphenyl)-4-(methylsulfanyl)-6H,7H-pyrazolo[1,5-a][1,3,5]triazin-7-one (75 mg, 0.239 mmol) and (3-aminopropyl)(benzyl)amine (117 mg, 0.71 mmol) were dissolved in Pyridine (1 mL). The reaction mixture was heated in a microwave at 140° C. for 2 h. The reaction mixture was evaporated to dryness and purified by preparative HPLC method A affording the title compound as a pale yellow oil (35 mg, 34%).

¹H NMR (500 MHz, Methanol-d4) δ 8.36 (s, 2H), 8.29-8.20 (m, 2H), 7.56-7.46 (m, 2H), 7.44-7.30 (m, 5H), 5.70 (s, 1H), 4.15 (s, 2H), 3.83 (t, J=6.3 Hz, 2H), 3.24-3.08 (m, 2H), 2.18 (dt, J=14.2, 6.5 Hz, 2H), 1.37 (s, 9H). LCMS Method B: rt 2.44 min, 100%; m/z 432 (MH⁺).

### 2-(4-tert-butylphenyl)-4-{[3-(dimethylamino)propyl]amino}-6H,7H-pyrazolo[1,5-a][1,3,5]triazin-7-one—Example 75

2-(4-tert-butylphenyl)-4-(methylsulfanyl)-6H,7H-pyrazolo[1,5-a][1,3,5]triazin-7-one (75 mg, 0.239 mmol) and (3-aminopropyl)dimethylamine (122 mg, 1.193 mmol) were dissolved in Pyridine (1 mL). The reaction mixture was heated in a microwave at 140° C. for 2 h. The reaction mixture was evaporated to dryness and purified by preparative HPLC method A affording the title compound as a pale yellow oil (14 mg, 15%).

¹H NMR (500 MHz, Methanol-d4) δ 8.42 (s, 2H), 8.28 (d, J=8.5 Hz, 2H), 7.54 (d, J=8.5 Hz, 2H), 5.71 (s, 1H), 3.85 (t, J=6.4 Hz, 2H), 3.27 (dd, J=9.2, 6.8 Hz, 2H), 2.85 (s, 6H), 2.27-2.14 (m, 2H), 1.38 (s, 9H). LCMS Method B: rt 2.20 min, 98%; m/z 369 (MH⁺).

### 2-(4-tert-butylphenyl)-4-{[3-(1,2,3,4-tetrahydroquinolin-1-yl)propyl]amino}-6H,7H-pyrazolo[1,5-a][1,3,5]triazin-7-one—Example 76

2-(4-tert-butylphenyl)-4-(methylsulfanyl)-6H,7H-pyrazolo[1,5-a][1,3,5]triazin-7-one (70 mg, 0.223 mmol) and 3-(1,2,3,4-tetrahydroquinolin-1-yl)propan-1-amine (84 mg, 0.446 mmol) were dissolved in Pyridine (1 mL). The reaction mixture was heated in a microwave at 140° C. for 3 h. The reaction mixture was evaporated to dryness and purified by preparative HPLC method A affording the title compound as a white solid (17 mg, 16%).

¹H NMR (500 MHz, Methanol-d4) δ 8.23 (d, J=8.6 Hz, 2H), 7.48 (d, J=8.6 Hz, 2H), 6.89 (t, J=7.7 Hz, 2H), 6.64 (d, J=8.0 Hz, 1H), 6.48 (t, J=7.3 Hz, 1H), 5.67 (s, 1H), 3.80 (t, J=7.2 Hz, 2H), 3.45 (t, J=7.0 Hz, 2H), 3.37-3.32 (m, 2H), 2.74 (t, J=6.3 Hz, 2H), 2.10 (p, J=7.1 Hz, 2H), 1.95 (dt, J=11.5, 6.3 Hz, 2H), 1.39 (s, 9H). LCMS Method B: rt 4.72 min, 97%; m/z 457 (MH⁺).

### 2-(4-tert-butylphenyl)-4-[4-(2-hydroxyethyl)piperazin-1-yl]-6H,7H-pyrazolo[1,5-a][1,3,5]triazin-7-one—Example 77

2-(4-tert-butylphenyl)-4-(methylsulfanyl)-6H,7H-pyrazolo[1,5-a][1,3,5]triazin-7-one (70 mg, 0.223 mmol) and 2-(piperazin-1-yl)ethan-1-ol (58 mg, 0.446 mmol) were dissolved in Pyridine (1 mL). The reaction mixture was heated in a microwave at 140° C. for 2 h. The reaction mixture was evaporated to dryness and purified by preparative HPLC method A affording the title compound as a white solid (35 mg, 39%).

¹H NMR (500 MHz, Methanol-d4) δ 8.28 (d, J=8.6 Hz, 2H), 7.54 (d, J=8.6 Hz, 2H), 5.73 (s, 1H), 4.51 (s, 4H), 3.80 (t, J=5.7 Hz, 2H), 2.94-2.82 (m, 4H), 2.75 (t, J=5.7 Hz, 2H), 1.40 (s, 9H). LCMS Method B: rt 2.22 min, 100%; m/z 397 (MH⁺).

### 2-(4-tert-butylphenyl)-4-{[3-(dibutylamino)propyl]amino}-6H,7H-pyrazolo[1,5-a][1,3,5]triazin-7-one—Example 78

2-(4-tert-butylphenyl)-4-(methylsulfanyl)-6H,7H-pyrazolo[1,5-a][1,3,5]triazin-7-one (50 mg, 0.159 mmol) and (3-aminopropyl)dibutylamine (148 mg, 0.79 mmol) were dissolved in Pyridine (1 mL). The reaction mixture was heated in a microwave at 140° C. for 10 h. The reaction mixture was evaporated to dryness filtered through a pad of silica (DCM/MeOH 10%). Filtrate was evaporated to dryness and purified by preparative HPLC method A affording the title compound as a colorless (8 mg, 10%).

¹H NMR (500 MHz, Chloroform-d) δ 8.50 (s, 1H), 8.28 (d, J=8.6 Hz, 2H), 7.83 (s, 1H), 7.44 (d, J=8.6 Hz, 2H), 5.82 (s, 1H), 3.79 (s, 2H), 3.20-3.07 (m, 2H), 2.99-2.85 (m, 4H), 2.20 (s, 2H), 1.57 (dq, J=12.0, 8.1, 6.3 Hz, 4H), 1.34 (s, 9H), 1.28 (q, J=7.4 Hz, 4H), 0.87 (t, J=7.3 Hz, 6H). LCMS Method B: rt 2.94 min, 98%; m/z 453 (MH⁺).

### 4-{[(4-benzylmorpholin-2-yl)methyl]amino}-2-(4-tert-butylphenyl)-6H,7H-pyrazolo[1,5-a][1,3,5]triazin-7-one—Example 79

2-(4-tert-butylphenyl)-4-(methylsulfanyl)-6H,7H-pyrazolo[1,5-a][1,3,5]triazin-7-one (40 mg, 0.127 mmol) and (4-benzylmorpholin-2-yl)methanamine (50 mg, 0.242 mmol) were dissolved in Pyridine (1 mL). The reaction mixture was heated in a microwave at 140° C. for 16 h. The reaction mixture was evaporated to dryness and purified by preparative HPLC method A affording the title compound as a beige solid (3 mg, 4%).

¹H NMR (500 MHz, Chloroform-d) δ 8.30 (d, J=8.4 Hz, 2H), 7.48 (d, J=8.5 Hz, 2H), 7.32 (dd, J=7.7, 3.3 Hz, 5H), 6.70 (s, 1H), 5.82 (s, 1H), 4.06 (s, 1H), 3.95 (d, J=10.0 Hz, 2H), 3.89 (t, J=10.9 Hz, 1H), 3.62 (m, 3H), 2.99 (d, J=11.0 Hz, 1H), 2.81 (d, J=11.3 Hz, 1H), 2.35 (td, J=11.1, 2.8 Hz, 1H), 2.16 (t, J=10.7 Hz, 1H), 1.39 (s, 9H).

### 2-(4-tert-butylphenyl)-4-[(3-hydroxy-3-methylbutyl)amino]-6H,7H-pyrazolo[1,5-a][1,3,5]triazin-7-one—Compound 80

2-(4-tert-butylphenyl)-4-(methylsulfanyl)-6H,7H-pyrazolo[1,5-a][1,3,5]triazin-7-one (80 mg, 0.25 mmol) and 4-amino-2-methylbutan-2-ol (131 mg, 1.27 mmol) were dissolved in pyridine (0.5 ml). Stirred at 40° C. for 14 h, then 70° C. for 6 h. The reaction mixture was evaporated to dryness and purified using HPLC method A to afford the title compound as a white solid (9 mg, 10%).

¹H NMR (500 MHz, Chloroform-d) δ 8.36 (d, J=8.5 Hz, 2H), 7.51 (d, J=8.5 Hz, 2H), 6.97 (s, 1H), 5.85 (s, 1H), 3.95 (q, J=5.9 Hz, 2H), 1.95 (t, J=6.5 Hz, 2H), 1.40 (s, 6H), 1.38 (s, 9H). LCMS Method B: rt 3.52 min, 100%; m/z 370.3 (MH⁺).

### 2-(4-tert-butylphenyl)-4-{[2-(morpholin-4-yl)ethyl]amino}-6H,7H-pyrazolo[1,5-a][1,3,5]triazin-7-one—Compound 81

2-(4-tert-butylphenyl)-4-(methylsulfanyl)-6H,7H-pyrazolo[1,5-a][1,3,5]triazin-7-one (60 mg, 0.19 mmol) was stirred in 2-(morpholin-4-yl)ethan-1-amine (500 mg, 3.84 mmol) at RT for 14 h. The reaction mixture was purified using HPLC method A to afford the title compound (43 mg, 56%).

¹H NMR (500 MHz, Methanol-d4) δ 8.26 (d, J=8.4 Hz, 2H), 8.17 (s, 2H), 7.52 (d, J=8.4 Hz, 2H), 5.69 (s, 1H), 3.97 (t, J=6.1 Hz, 2H), 3.80-3.67 (m, 4H), 3.02 (t, J=6.1 Hz, 2H), 2.91 (m, 4H), 1.37 (s, 9H). LCMS Method B: rt 2.19 min, 97%; m/z 397.2 (MH⁺).

### 2-(4-tert-butylphenyl)-4-{[2-(piperidin-1-yl)ethyl]amino}-6H,7H-pyrazolo[1,5-a][1,3,5]triazin-7-one—Compound 82

2-(4-tert-butylphenyl)-4-(methylsulfanyl)-6H,7H-pyrazolo[1,5-a][1,3,5]triazin-7-one (60 mg, 0.19 mmol) was stirred in 2-(piperidin-1-yl)ethan-1-amine (500 mg, 3.9 mmol) at RT for 14 h. The reaction mixture was purified using HPLC method A to afford the title compound (52 mg, 67%).

¹H NMR (500 MHz, Methanol-d4) δ 8.34 (s, 2H, formic acid salt), 8.26 (d, J=8.5 Hz, 2H), 7.53 (d, J=8.5 Hz, 2H), 5.73 (s, 1H), 4.13 (t, J=5.8 Hz, 2H), 3.45 (t, J=5.8 Hz, 2H), 3.43-3.33 (m, 4H), 1.80-1.71 (m, 4H), 1.66-1.58 (m, 2H), 1.37 (s, 9H). LCMS Method B: rt 2.34 min, 97%; m/z 395.2 (MH⁺).

### 2-(4-tert-butylphenyl)-4-({2-[cyclohexyl(methyl)amino]ethyl}amino)-6H,7H-pyrazolo[1,5-a][1,3,5]triazin-7-one—Compound 83

2-(4-tert-butylphenyl)-4-(methylsulfanyl)-6H,7H-pyrazolo[1,5-a][1,3,5]triazin-7-one (60 mg, 0.19 mmol) and N-cyclohexyl-N-methylethane-1,2-diamine (149.11 mg, 0.95 mmol) were dissolved in pyridine (0.5 mL), stirred at RT for 14 h, then at 120° C. for 2 h in microwave. Triethylamine (132 μl, 0.95 mmol) was added and the reaction stirred at RT for 96 h, followed by heating at 120° C. for 1 h in microwave. The reaction mixture was evaporated to dryness and purified using HPLC method A to afford the title compound as a bis formic acid salt (10 mg, 12%).

¹H NMR (500 MHz, Methanol-d4) δ 8.40 (s, 2H), 8.26 (d, J=8.5 Hz, 2H), 7.53 (d, J=8.6 Hz, 2H), 5.72 (s, 1H), 4.18-4.08 (m, 2H), 3.56-3.48 (m, 2H), 3.39 (t, J=11.8 Hz, 1H), 2.90 (s, 3H), 1.96 (d, J=10.9 Hz, 2H), 1.84 (d, J=13.2 Hz, 2H), 1.70-1.60 (m, 1H), 1.51-1.40 (m, 2H), 1.37 (s, 9H), 1.34-1.24 (m, 2H), 1.22-1.11 (m, 1H). LCMS Method B: rt 2.60 min, 99%; m/z 423.2 (MH⁺).

### 4-({[2-(4-tert-butylphenyl)-7-oxo-6H,7H-pyrazolo[1,5-a][1,3,5]triazin-4-yl]amino}methyl)benzonitrile—Compound 84

2-(4-tert-butylphenyl)-4-(methylsulfanyl)-6H,7H-pyrazolo[1,5-a][1,3,5]triazin-7-one (80 mg, 0.25 mmol) and 4-(aminomethyl)benzonitrile (202 mg, 1.53 mmol) were dissolved in pyridine (0.5 mL) and stirred at 50° C. for 36 h. 4-(aminomethyl)benzonitrile (202 mg, 1.53 mmol) was added and the reaction stirred at 50° C. for 14 h. The reaction mixture was evaporated to dryness and purified using HPLC method A to afford the title compound (6 mg, 6%).

¹H NMR (500 MHz, Methanol-d4) δ 8.30-8.12 (m, 2H), 7.74 (d, J=8.4 Hz, 2H), 7.68 (d, J=8.4 Hz, 2H), 7.54-7.43 (m, 2H), 5.70 (s, 1H), 4.98 (s, 2H), 1.38 (s, 9H). LCMS Method B: rt 3.95 min, 96%; m/z 399.1 (MH⁺).

Example 85 was synthesized by reacting Compound F with Compound BA. The synthetic route for Compound BA is illustrated in Scheme 19.

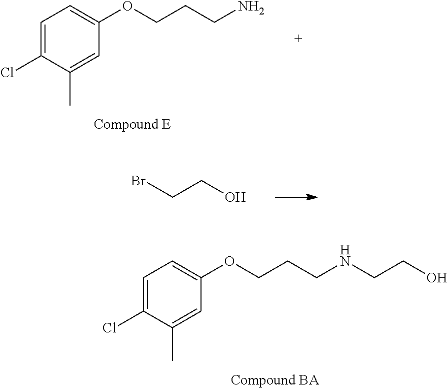

### 2-{[3-(4-chloro-3-methylphenoxy)propyl]amino}ethan-1-ol—Compound BA

2-bromoethan-1-ol (227 μl, 3.20 mmol) and 4-(3-aminopropoxy)-1-chloro-2-methylbenzene (895 mg, 4.48 mmol) were stirred in acetonitrile (5 mL) under nitrogen, and K₂CO₃ (708 mg, 5.12 mmol) was added. The reaction was stirred at 40° C. for 14 h, followed by 60° C. for 5 h. The reaction was filtered and washed with acetonitrile, the filtrate collected and concentrated. The crude product was purified by HPLC method B, affording the title compound as a colourless oil (173 mg, 92% purity, 20%).

¹H NMR (500 MHz, Chloroform-d) δ 8.54 (s, 1H), 7.20 (d, J=8.7 Hz, 1H), 6.76 (d, J=2.9 Hz, 1H), 6.65 (dd, J=8.7, 3.0 Hz, 1H), 4.01 (t, J=5.9 Hz, 2H), 3.78 (dd, J=5.8, 4.4 Hz, 2H), 3.01 (t, J=7.1 Hz, 2H), 2.98-2.94 (m, 2H), 2.32 (s, 3H), 2.10 (p, J=6.4 Hz, 2H). LCMS Method A: rt 1.50 min, 92%; m/z 244.2 (MH⁺).

### 2-(4-tert-butylphenyl)-4-({[3-(4-chloro-3-methylphenoxy)propyl](2-hydroxyethyl)amino}-6H,7H-pyrazolo[1,5-a][1,3,5]triazin-7-one—Example 85

2-{[3-(4-chloro-3-methylphenoxy)propyl]amino}ethan-1-ol (120 mg, 0.38 mmol) and 2-(4-tert-butylphenyl)-4-(methylsulfanyl)-6H,7H-pyrazolo[1,5-a][1,3,5]triazin-7-one (172.4 mg, 0.71 mmol) were stirred in Pyridine (0.5 mL) in a microwave at 130° C. for 18 h. The reaction mixture was then concentrated, and purified by HPLC method A affording the title compound as a pale yellow gum (1 mg, 75% purity, 1%).

¹H NMR (500 MHz, Chloroform-d) δ 8.31 (d, J=8.5 Hz, 2H), 7.48 (d, J=8.5 Hz, 2H), 7.23 (d, J=8.7 Hz, 1H), 6.81 (d, J=2.9 Hz, 1H), 6.70 (dd, J=8.7, 2.9 Hz, 1H), 5.83 (s, 1H), 4.26 (t, J=5.0 Hz, 2H), 4.14-4.07 (m, 4H), 4.03 (t, J=5.1 Hz, 2H), 2.34 (s, 3H), 1.38 (s, 9H), 1.28 (s, 2H). LCMS Method B: rt 4.67 min, 75%; m/z 510.3 (MH⁺).

Example 86 was synthesized by reacting Compound F with Compound BD. The synthetic route for Compound BD is illustrated in Scheme 20.

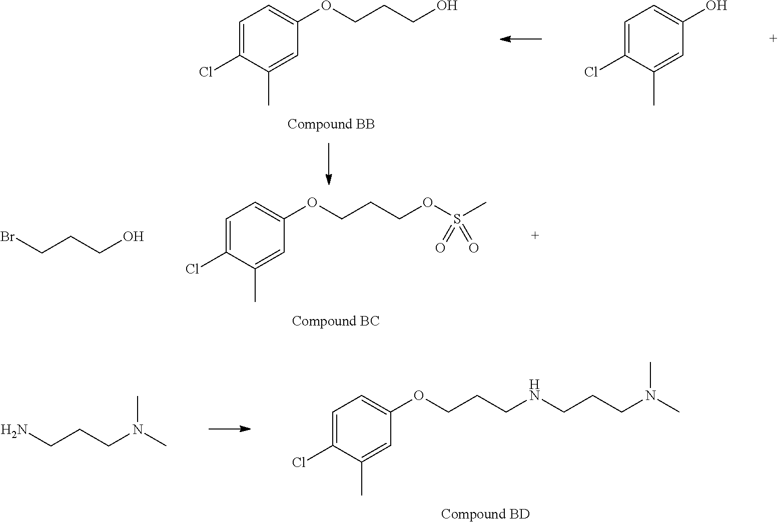

### 3-(4-chloro-3-methylphenoxy)propan-1-ol—Compound BB

3-bromopropan-1-ol (0.95 mL, 10.52 mmol), 4-chloro-3-methylphenol (1.5 g, 10.52 mmol) and Cs₂CO₃ (6.86 g, 21.04 mmol) were stirred in acetonitrile (40 mL) in a pressure tube at 50° C. for 14 h. The reaction was concentrated and partitioned between ethyl acetate and water; the combined organics were dried over Na₂SO₄, and concentrated. The crude product was purified by Biotage FCC (25 g SNAP KP-SiO₂, 50-100% EtOAc in Heptanes) affording the title compound as a colourless oil (1.49 g, 91% purity, 64%).

¹H NMR (250 MHz, Chloroform-d) δ 7.22 (d, J=8.7 Hz, 1H), 6.78 (d, J=2.9 Hz, 1H), 6.68 (dd, J=8.7, 3.0 Hz, 1H), 4.09 (t, J=5.9 Hz, 2H), 3.86 (t, J=5.9 Hz, 2H), 2.34 (s, 3H), 2.08-1.99 (m, 2H). LCMS Method C: rt 1.12 min, 91%; m/z 201.0 (MH⁺).

### 3-(4-chloro-3-methylphenoxy)propyl methanesulfonate—Compound BC

3-(4-chloro-3-methylphenoxy)propan-1-ol (1.49 g, 7.47 mmol) and triethylamine (1.24 ml, 8.96 mmol) were dissolved in DCM (15 mL) and cooled in an ice bath. Methanesulfonyl chloride (0.61 mL, 7.84 mmol) in DCM (5 mL) was added dropwise. The reaction was allowed to warm to RT and stirred for 14 h. The reaction was partitioned between DCM and water, washing the aqueous with DCM; the combined organics were passed through a hydrophobic frit, and concentrated, affording the title compound as a colourless oil (2.02 g, 69%).

¹H NMR (500 MHz, Chloroform-d) δ 7.22 (d, J=8.7 Hz, 1H), 6.77 (d, J=2.9 Hz, 1H), 6.67 (dd, J=8.7, 3.0 Hz, 1H), 4.44 (t, J=6.1 Hz, 2H), 4.05 (t, J=5.9 Hz, 2H), 2.99 (s, 3H), 2.34 (s, 3H), 2.24-2.18 (m, 2H).

### [3-(4-chloro-3-methylphenoxy)propyl][3-(dimethylamino)propyl]amine—Compound BD

3-(4-chloro-3-methylphenoxy)propyl methanesulfonate (1000 mg, 3.59 mmol) and K₂CO₃ (595 mg, 4.30 mmol) were stirred in acetonitrile (20 mL) and cooled over ice. N,N-dimethylpropane-1,3-diamine (550 mg, 5.38 mmol) was then added dropwise to the reaction. The reaction was allowed to warm to RT and stirred for 14 h, followed by heating at 50° C. for 7 h. The reaction was concentrated and purified by column chromatography using Biotage FCC (25 g SNAP KP-SiO₂, 50-100% EtOAc in Heptanes; then 0-35% MeOH in EtOAc) affording the title compound as a pale orange gum (410 mg, 40%).

¹H NMR (500 MHz, Chloroform-d) δ 7.22 (d, J=8.7 Hz, 1H), 6.77 (d, J=2.9 Hz, 1H), 6.66 (dd, J=8.7, 2.9 Hz, 1H), 4.03 (t, J=5.7 Hz, 2H), 3.16 (t, J=6.1 Hz, 2H), 3.12 (t, J=7.0 Hz, 2H), 2.61 (t, J=6.0 Hz, 2H), 2.33 (s, 3H), 2.27 (s, 6H), 2.22 (p, J=6.9 Hz, 2H), 1.95 (p, J=6.0 Hz, 2H). LCMS Method C: rt 0.69 min, 99%; m/z 285.0 (MH⁺).

### 2-(4-tert-butylphenyl)-4-{[3-(4-chloro-3-methylphenoxy)propyl][3-(dimethylamino) propyl]amino}-6H,7H-pyrazolo[1,5-a][1,3,5]triazin-7-one—Example 86

[3-(4-chloro-3-methylphenoxy)propyl][3-(dimethylamino)propyl]amine (200 mg, 0.64 mmol) and

2-(4-tert-butylphenyl)-4-(methylsulfanyl)-6H,7H-pyrazolo[1,5-a][1,3,5]triazin-7-one (410 mg, 1.44 mmol) were dissolved in pyridine (0.8 mL). The reaction was heated in a microwave at 130° C. for 10 h, followed by heating at 150° C. for 14 h. The reaction was concentrated and purified by HPLC method A affording the title compound as a yellow gum (45 mg, 13%).

¹H NMR (500 MHz, DMSO-d6) δ 8.22 (d, J=8.5 Hz, 2H), 7.45 (d, J=8.4 Hz, 2H), 7.25 (d, J=8.7 Hz, 1H), 6.89 (d, J=2.6 Hz, 1H), 6.78 (dd, J=8.7, 2.8 Hz, 1H), 5.66 (s, 1H), 4.09 (t, J=6.0 Hz, 2H), 3.33 (m, 4H), 2.39 (t, J=6.9 Hz, 2H), 2.23 (s, 3H), 2.21 (s, 6H), 2.19-2.14 (m, 2H), 1.90 (p, J=7.0 Hz, 2H), 1.31 (s, 9H). LCMS Method B: rt 3.38 min, 99%; m/z 551.2 (MH⁺).

Example 87 was synthesized by reacting Compound F with Compound BG. The synthetic route for Compound BG is illustrated in Scheme 21.

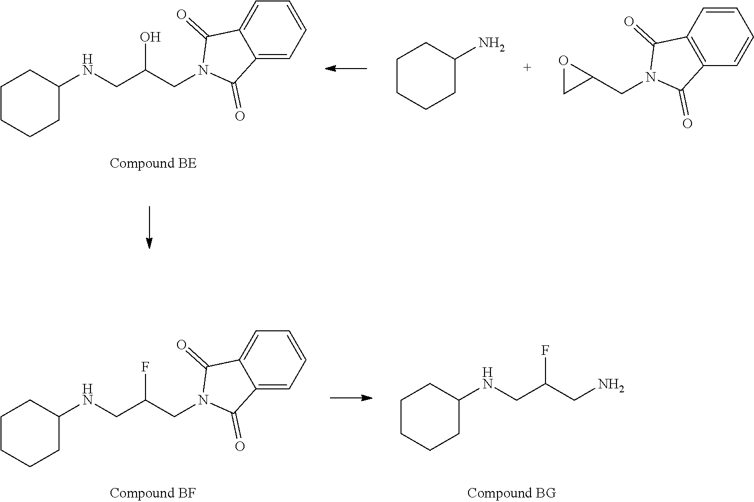 [SMILES: O=C1c2ccccc2C(=O)N1CC(O)CNC1CCCCC1 ; NC1CCCCC1 ; O=C1c2ccccc2C(=O)N1CC1CO1 ; O=C1c2ccccc2C(=O)N1CC(F)CNC1CCCCC1 ; NCC(F)CNC1CCCCC1]

### 2-[3-(cyclohexylamino)-2-hydroxypropyl]-2,3-dihydro-1H-isoindole-1,3-dione—Compound BE

2-(oxiran-2-ylmethyl)-2,3-dihydro-1H-isoindole-1,3-dione (1.5 g, 7.38 mmol) and cyclohexylamine (0.73 g, 0.73 mmol) were dissolved in isopropanol (25 mL). The reaction was stirred at 85° C. for 14 h. The reaction was allowed to cool to RT. The reaction was filtered and the solid was dried under vacuum affording the title compound as a white solid (1.17 g, 92% purity, 52%)

¹H NMR (500 MHz, DMSO-d6) δ 7.91-7.80 (m, 4H), 4.93 (d, J=4.8 Hz, 1H), 3.87-3.80 (m, 1H), 3.64-3.51 (m, 2H), 2.58 (dd, J=11.7, 4.8 Hz, 1H), 2.35-2.25 (m, 1H), 1.76 (t, J=10.9 Hz, 2H), 1.64 (d, J=12.9 Hz, 2H), 1.54 (dd, J=8.4, 3.6 Hz, 1H), 1.42 (s, 1H), 1.24-1.08 (m, 3H), 1.02-0.91 (m, 2H). LCMS Method C: rt 0.78 min, 92%; m/z 302.95 (MH⁺).

### 2-[3-(cyclohexylamino)-2-fluoropropyl]-2,3-dihydro-1H-isoindole-1,3-dione—Compound BF

2-[3-(cyclohexylamino)-2-hydroxypropyl]-2,3-dihydro-1H-isoindole-1,3-dione (500 mg, 1.65 mmol) was dissolved in DCM (25 mL) and cooled to −78° C. under nitrogen. 2.7 M DeoxoFluor in toluene (1.84 mL, 4.96 mmol) was added dropwise. The reaction was stirred at −78° C. for 1 hr, allowed to warm to RT and stirred for 110 h. 2.7 M DeoxoFluor in toluene (1.84 mL, 4.96 mmol) was added and the reaction stirred at RT for 14 h followed by heating at 40° C. for 20 h. The DCM was replaced with DCE (10 mL) and 2.7 M Deoxofluor in toluene (0.92 mL, 2.48 mmol) was added. The reaction was heated at 80° C. for 38 h followed by stirring at RT for 96 h. The reaction was poured over ice and taken to pH 9 using sat. aq. K₂CO₃ and extracted with DCM. The combined organics were dried over Na₂SO₄ and concentrated. The product was purified by column chromatography using Biotage FCC (25 g SNAP KP-SiO₂, 0-10% MeOH in DCM) affording the title compound as a brown oil (400 mg, 31% purity, 24%)

LCMS Method C: rt 0.81 min, 31%; m/z 304.95 (MH⁺).

### N-(3-amino-2-fluoropropyl)cyclohexanamine—Compound BG

2-[3-(cyclohexylamino)-2-fluoropropyl]-2,3-dihydro-1H-isoindole-1,3-dione (200 mg, 0.66 mmol) was dissolved in EtOH (10 ml) and hydrazine monohydrate (128.12 μL, 2.63 mmol) was added. The reaction was heated at 60° C. for 4 h. The reaction was concentrated, dissolved in MeOH. This was followed by filtration through a 2 g SCX-2 column, washing with methanol and eluting with 0.7M NH₃ in methanol. The basic eluent was concentrated, affording the title compound that was used directly in the next step (76 mg, ˜70% purity, 46%)

LCMS Method C: rt 0.18 min, m/z 175.00 (MH⁺)

### 2-(4-tert-butylphenyl)-4-{[3-(cyclohexylamino)-2-fluoropropyl]amino}-6H,7H-pyrazolo[1,5-a][1,3,5]triazin-7-one—Compound 87

N-(3-amino-2-fluoropropyl)cyclohexanamine (76 mg, 0.31 mmol) and 2-(4-tert-butylphenyl)-4-(methylsulfanyl)-6H,7H-pyrazolo[1,5-a][1,3,5]triazin-7-one (60 mg, 0.19 mmol) were dissolved in pyridine (0.5 mL). The reaction was heated in a microwave at 120° C. for 3 h. The reaction was concentrated and purified by HPLC method A affording the title compound 87 (7 mg, 94% purity, 8%).

¹H NMR (500 MHz, DMSO-d6) δ 8.39 (s, 1H), 8.34-8.20 (m, 2H), 8.17 (s, 1H), 7.54-7.42 (m, 2H), 5.71 (s, 1H), 5.03-4.75 (m, 1H), 3.91-3.82 (m, 2H), 2.98-2.78 (m, 3H), 1.82 (t, 2H), 1.69-1.59 (m, 2H), 1.57-1.46 (m, 1H), 1.31 (s, 9H), 1.23-1.09 (m, 2H), 1.10-0.94 (m, 3H). LCMS Method B: rt 2.58 min, 94%; m/z 441.3 (MH⁺)

Example 88 was synthesized by reacting Compound F with Compound BI. The synthetic route for Compound BI is illustrated in Scheme 22.

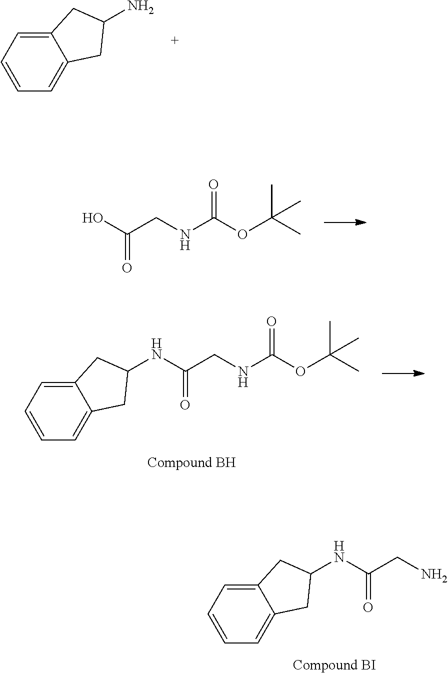 [SMILES: NC1Cc2ccccc2C1 ; CC(C)(C)OC(=O)NCC(=O)O ; CC(C)(C)OC(=O)NCC(=O)NC1Cc2ccccc2C1 ; NCC(=O)NC1Cc2ccccc2C1]

### tert-butyl N-{[(2,3-dihydro-1H-inden-2-yl)carbamoyl]methyl}carbamate—Compound BH

2-{[(tert-butoxy)carbonyl]amino}acetic acid (1 g, 5.71 mmol), N-(3-Dimethylaminopropyl)-N′-ethylcarbodiimide hydrochloride (2.63 g, 13.7 mmol), 1-Hydroxy-7-azabenzotriazole (1.71 g, 12.56 mmol) and triethylamine (3.18 mL, 22.83 mmol) were dissolved in DCE (10 mL) and the reaction was stirred at RT for 0.5 h. 2,3-dihydro-1H-inden-2-amine (1.14 mL 6.85 mmol) was added and the reaction was stirred at RT for 14 h. The reaction was diluted with water and extracted with DCM. The combined organics were dried over Na₂SO₄ and purified by column chromatography using Biotage FCC (50 g SNAP KP-SiO₂, 0-50% EtOAc in Heptanes) affording the title compound (1.17 g, 85% purity, 69%)

¹H NMR (500 MHz, Methanol-d4) δ 7.25-7.16 (m, 2H), 7.16-6.99 (m, 2H), 4.68-4.52 (m, 1H), 3.66 (s, 2H), 3.24 (dd, J=15.8, 7.4 Hz, 2H), 2.85 (dd, J=15.8, 5.6 Hz, 2H), 1.43 (s, 9H). LCMS Method C: rt 1.04 min 85%; m/z 235.1 (MH⁺-tert-butyl)

### 2-amino-N-(2,3-dihydro-1H-inden-2-yl)acetamide—Compound BI

tert-butyl N-{[(2,3-dihydro-1H-inden-2-yl)carbamoyl]methyl}carbamate (1.17 g, 4.03 mmol) was dissolved in 4 M HCl in dioxane (8 mL) and the reaction was stirred at RT for 3 h. The reaction was concentrated affording the title compound as an off-white solid (913 mg, 94% purity, 100%)

¹H NMR (500 MHz, DMSO-d6) δ 8.70 (s, 1H), 8.05 (s, 2H), 7.33-7.20 (m, 2H), 7.17 (dd, J=5.4, 3.2 Hz, 2H), 4.69-4.35 (m, 1H), 3.50 (s, 2H), 3.22 (dd, J=16.0, 7.4 Hz, 2H), 2.80 (dd, J=16.0, 5.0 Hz, 2H). LCMS Method A: rt 1.25 min, 94%; m/z 191.2 (MH⁺)

### 2-{[2-(4-tert-butylphenyl)-7-oxo-6H,7H-pyrazolo[1,5-a][1,3,5]triazin-4-yl]amino}-N-(2,3-dihydro-1H-inden-2-yl)acetamide—Example 88

2-amino-N-(2,3-dihydro-1H-inden-2-yl)acetamide (173 mg, 0.76 mmol) and 2-(4-tert-butylphenyl)-4-(methylsulfanyl)-6H,7H-pyrazolo[1,5-a][1,3,5]triazin-7-one (80 mg, 0.25 mmol) were dissolved in pyridine (0.5 mL). The reaction was stirred at RT for 110 h. Further 2-amino-N-(2,3-dihydro-1H-inden-2-yl)acetamide (100 mg, 0.44 mmol) and triethylamine (105 μL, 0.76 mmol) were added and the reaction stirred at RT for 14 h. The reaction was concentrated and purified by HPLC method A affording the title compound (5 mg, 95% purity, 4%).

¹H NMR (500 MHz, Methanol-d4) δ 8.55 (s, 1H), 8.23 (d, J=8.6 Hz, 2H), 7.47 (d, J=8.6 Hz, 2H), 7.17-7.07 (m, 4H), 5.59 (s, 1H), 4.74-4.58 (m, 1H), 4.27 (s, 2H), 3.23 (dd, J=15.9, 7.5 Hz, 2H), 2.88 (dd, J=15.9, 5.6 Hz, 2H), 1.37 (s, 9H). LCMS Method B: rt 3.76 min, 95%; m/z 457.2 (MH⁺)

Example 89 was synthesized by reacting Compound F with Compound BL. The synthetic route for Compound BL is illustrated in Scheme 23.

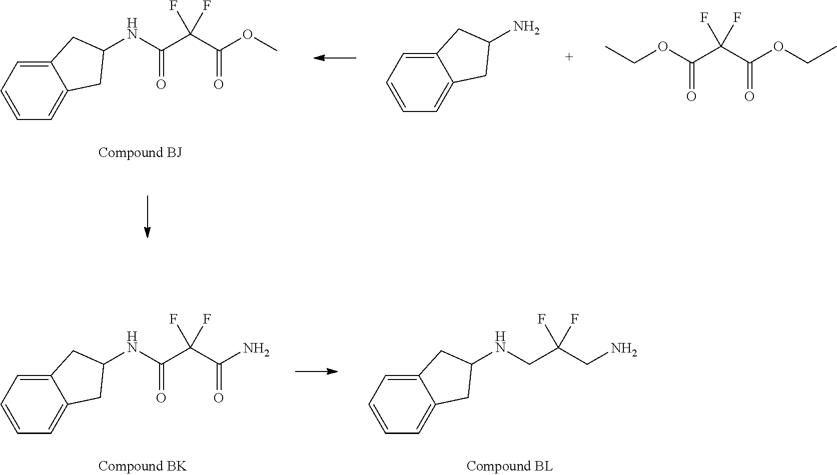 [SMILES: COC(=O)C(F)(F)C(=O)NC1Cc2ccccc2C1 ; NC1Cc2ccccc2C1 ; CCOC(=O)C(F)(F)C(=O)OCC ; NC(=O)C(F)(F)C(=O)NC1Cc2ccccc2C1 ; NCC(F)(F)CNC1Cc2ccccc2C1]

### methyl 2-[(2,3-dihydro-1H-inden-2-yl)carbamoyl]-2,2-difluoroacetate—Compound BJ

Diethyl difluoropropanedioate (2.87 ml, 17.23 mmol) was dissolved in MeOH (20 mL) and a solution of 2,3-dihydro-1H-inden-2-amine (2.07 g, 15.51 mmol) in MeOH (15 mL) was added dropwise. The reaction was stirred at RT for 14 h. The reaction was concentrated and purified by Biotage FCC (50 g SNAP KP-SiO₂, 0-50% EtOAc in Heptanes) affording the title compound as a pale brown solid (1.79 g, 91% purity, 35%)

¹H NMR (500 MHz, DMSO-d6) δ 9.48 (d, J=6.9 Hz, 1H), 7.22 (dd, J=5.3, 3.4 Hz, 2H), 7.15 (dd, J=5.5, 3.2 Hz, 2H), 4.60-4.48 (m, 1H), 3.88 (s, 3H), 3.20 (dd, J=15.9, 7.9 Hz, 2H), 2.91 (dd, J=15.9, 6.6 Hz, 2H). LCMS Method C: rt 1.09 min, 91%, m/z 269.95 (MH⁺)

### N-(2,3-dihydro-1H-inden-2-yl)-2,2-difluoropropanediamide—Compound BK

Methyl 2-[(2,3-dihydro-1H-inden-2-yl)carbamoyl]-2,2-difluoroacetate (1.79 g, 6.65 mmol) was dissolved in MeOH (5 mL) and cooled in an ice bath. 7N NH₃ in MeOH (10 mL) was added dropwise. The reaction was allowed to warm to RT and stirred for 14 h. The reaction was concentrated affording the title compound as a pale brown solid (1.69 g, 93% purity, 100%)

¹H NMR (500 MHz, DMSO-d6) δ 9.16 (d, J=7.0 Hz, 1H), 8.28 (s, 1H), 8.12 (s, 1H), 7.22 (dd, J=5.3, 3.4 Hz, 2H), 7.16 (dd, J=5.5, 3.2 Hz, 2H), 4.53 (h, J=7.2 Hz, 1H), 3.18 (dd, J=15.8, 7.9 Hz, 2H), 2.92 (dd, J=15.8, 6.8 Hz, 2H). LCMS Method C: rt 0.92 min, 93%; m/z 254.95 (MH⁺)

### N-(3-amino-2,2-difluoropropyl)-2,3-dihydro-1H-inden-2-amine—Compound BL

1M Borane-THF solution (31 mL, 31 mmol) was cooled in an ice bath and a solution of N-(2,3-dihydro-1H-inden-2-yl)-2,2-difluoropropanediamide (1.59 g, 6.25 mmol) in THF (15 mL) was added dropwise. The reaction was allowed to warm to RT followed by heating at 75° C. for 14 h. The reaction was cooled with an ice bath and quenched with MeOH (15 mL). The reaction was concentrated and then dissolved in EtOH (10 mL). The reaction was acidified with conc. HCl and stirred at RT for 1 h. The solid formed was collected by filtration and washed with EtOH affording the title compound as a white solid (1.66 g, 84%)

¹H NMR (500 MHz, DMSO-d6) δ 10.04 (s, 1H), 8.79 (s, 2H), 7.41-7.04 (m, 4H), 4.11 (s, 1H), 3.85 (d, J=15.1 Hz, 2H), 3.70 (t, J=15.5 Hz, 2H), 3.32-3.27 (m, 2H), 3.21 (s, 2H).

### 2-(4-tert-Butylphenyl)-4-({3-[(2,3-dihydro-1H-inden-2-yl)amino]-2,2-difluoropropyl}amino)-6H,7H-pyrazolo[1,5-a][1,3,5]triazin-7-one—Example 89

N-(3-amino-2,2-difluoropropyl)-2,3-dihydro-1H-inden-2-amine (228 mg, 0.76 mmol), and

2-(4-tert-butylphenyl)-4-(methylsulfanyl)-6H,7H-pyrazolo[1,5-a][1,3,5]triazin-7-one (80 mg, 0.25 mmol) and triethylamine (212 μL, 1.53 mmol) were dissolved in pyridine (0.5 mL). The reaction was stirred at RT for 38 h then heated in a microwave at 120° C. for 4 h. The reaction was concentrated and purified by HPLC method A affording the title compound (15 mg, 12%).

¹H NMR (500 MHz, Methanol-d4) δ 8.25 (m, 3H), 7.50 (d, J=8.6 Hz, 2H), 7.05 (s, 4H), 5.73 (s, 1H), 4.28 (t, J=13.0 Hz, 2H), 3.75-3.56 (m, 1H), 3.30-3.18 (m, 2H), 3.06 (dd, J=15.6, 7.4 Hz, 2H), 2.73-2.64 (m, 2H), 1.36 (s, 9H). LCMS Method B: rt 2.76 min, 99%; m/z 493.2 (MH⁺)

Example 90 was synthesized by reacting Compound F with Compound BN. The synthetic route for Compound BN is illustrated in Scheme 24.

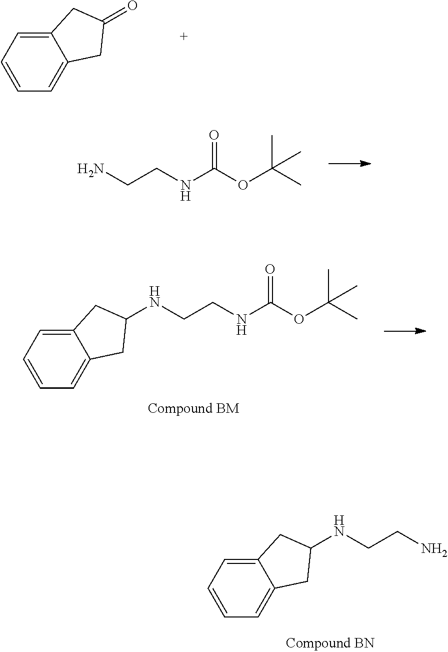 [SMILES: O=C1Cc2ccccc2C1 ; CC(C)(C)OC(=O)NCCN ; CC(C)(C)OC(=O)NCCNC1Cc2ccccc2C1 ; NCCNC1Cc2ccccc2C1]

### tert-Butyl N-{2-[(2,3-dihydro-1H-inden-2-yl)amino]ethyl}carbamate—Compound BM

tert-Butyl (2-aminoethyl)carbamate (1.21 g, 7.57 mmol), 2,3-dihydro-1H-inden-2-one and acetic acid (144 μL, 7.57 mmol) were dissolved in DCE (20 mL). Sodium triacetoxyborohydride (2.97 g, 14.0 mmol) was added and the reaction was stirred at RT for 1 h. The reaction was quenched with 1M NaOH (aq.) and extracted with tert-butyl methyl ether. The combined organics were dried over Na₂SO₄ and concentrated. The product was purified using column chromatography using Biotage FCC (25 g SNAP KP-SiO₂, 50-100% EtOAC in Heptanes) affording the title compound (1.4 g, 66%)

¹H NMR (500 MHz, DMSO-d6) δ 7.16 (dd, J=5.2, 3.4 Hz, 2H), 7.09 (dd, J=5.5, 3.2 Hz, 2H), 6.72 (s, 1H), 3.52-3.43 (m, 1H), 3.09-2.93 (m, 4H), 2.70-2.53 (m, 4H), 1.37 (s, 9H). LCMS Method C: rt 0.80 min, 98%; m/z 277.05 (MH⁺)

### N-(2-Aminoethyl)-2,3-dihydro-1H-inden-2-amine—Compound BN

tert-Butyl N-{2-[(2,3-dihydro-1H-inden-2-yl)amino]ethyl}carbamate (1.14 g, 5.07 mmol) was dissolved in 20% TFA in DCM (14 mL). The reaction was stirred at RT for 4.5 h. The reaction was concentrated. The product was dissolved in DCM and taken to pH 9 using sat. aq. NaHCO₃. The organic layer was removed and the aqueous was extracted with further DCM. The combined organics were dried over Na₂SO₄ and concentrated affording the title compound (183 mg, 18%)

¹H NMR (500 MHz, Methanol-d4) δ 7.23-7.13 (m, 2H), 7.10 (dd, J=5.5, 3.2 Hz, 2H), 3.70-3.53 (m, 1H), 3.38 (s, 1H), 3.18 (dd, J=15.6, 7.3 Hz, 2H), 2.90 (t, J=6.6 Hz, 1H), 2.86-2.69 (m, 4H). LCMS Method A: rt 1.32 min, 95%; m/z 177.2 (MH⁺)

### 2-(4-tert-Butylphenyl)-4-({2-[(2,3-dihydro-1H-inden-2-yl)amino]ethyl}amino)-6H,7H-pyrazolo[1,5-a][1,3,5]triazin-7-one—Example 90

N-(2-Aminoethyl)-2,3-dihydro-1H-inden-2-amine (180 mg, 0.76 mmol), 2-(4-tert-butylphenyl)-4-(methylsulfanyl)-6H,7H-pyrazolo[1,5-a][1,3,5]triazin-7-one (80 mg, 0.25 mmol) and triethylamine (212 μL, 1.53 mmol) were dissolved in pyridine (0.5 mL). The reaction was stirred at RT for 182 h. The reaction was concentrated and purified by HPLC method A affording the title compound (6 mg, 5%).

¹H NMR (500 MHz, Methanol-d4) δ 8.41 (s, 2H), 8.23 (d, J=8.5 Hz, 2H), 7.48 (d, J=8.5 Hz, 2H), 7.17 (s, 4H), 4.28-4.19 (m, 1H), 4.08 (t, J=5.4 Hz, 2H), 3.49 (t, J=5.4 Hz, 2H), 3.39-3.32 (m, 2H), 3.04 (dd, J=16.2, 6.1 Hz, 2H), 1.36 (s, 9H). LCMS Method B: rt 2.65 min, 99%; m/z 443.2 (MH⁺)

Example 91 was synthesized by reacting Compound F with Compound BP. The synthetic route for Compound BP is illustrated in Scheme 25.

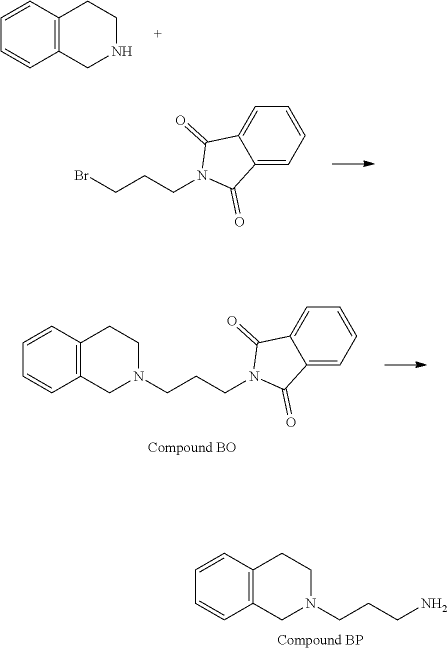 [SMILES: c1ccc2c(c1)CCNC2 ; O=C1c2ccccc2C(=O)N1CCCBr ; O=C1c2ccccc2C(=O)N1CCCN1CCc2ccccc2C1 ; NCCCN1CCc2ccccc2C1]

### 2-[3-(1,2,3,4-tetrahydroisoquinolin-2-yl)propyl]-2,3-dihydro-1H-isoindole-1,3-dione—Compound BO

2-(3-bromopropyl)-2,3-dihydro-1H-isoindole-1,3-dione (5 g, 18.65 mmol) in toluene (30 mL) was added dropwise to a warm solution of 1,2,3,4-tetrahydroisoquinoline (4.97 g, 37.3 mmol) in toluene (30 mL). The reaction mixture was heated at 120° C. for 16 h, cooled to 0° C. and filtered. The solid was washed with cold toluene. The filtrate was evaporated to dryness to give the title compound as an oil that solidified upon standing (7.0 g, 79% purity, 92%).

¹H NMR (500 MHz, Chloroform-d) δ 7.81-7.74 (m, 2H), 7.69-7.62 (m, 2H), 7.13-7.05 (m, 2H), 7.05-6.95 (m, 2H), 3.84 (t, J=7.0 Hz, 2H), 3.60 (s, 2H), 2.81 (t, J=5.9 Hz, 2H), 2.71 (t, J=5.9 Hz, 2H), 2.63 (t, J=7.0 Hz, 2H), 2.01 (p, J=7.0 Hz, 2H). LCMS Method C: rt 0.94 min, 79%; m/z 321 (MH⁺).

### 3-(1,2,3,4-tetrahydroisoquinolin-2-yl)propan-1-amine—Compound BP

2-[3-(1,2,3,4-tetrahydroisoquinolin-2-yl)propyl]-2,3-dihydro-1H-isoindole-1,3-dione (6.3 g, 15.53 mmol) was dissolved in Ethanol (10 mL) and hydrazine monohydrate (0.757 mL, 15.5 mmol) was added dropwise. The reaction was heated at 85° C. for 2 h. Hydrazine monohydrate (0.757 mL, 15.5 mmol) was added and reaction was heated for another 2 h at 85° C. Ethanol (5 mL) and HCl (6N, 15.5 mmol) were added and mixture was heated at 100° C. for 1 h. The precipitate was filtered off and washed with water. Combined filtrates were basified with 2M aq. Na₂CO₃ and extracted with TBME to give the title compound (2.96 g, 85% purity, 74%).

¹H NMR (500 MHz, Chloroform-d) δ 7.13-7.07 (m, 3H), 7.04-6.99 (m, 1H), 3.63 (s, 2H), 2.90 (t, J=5.9 Hz, 2H), 2.80 (t, J=6.8 Hz, 2H), 2.73 (t, J=5.9 Hz, 2H), 2.60-2.55 (m, 2H), 1.75 (p, J=6.9 Hz, 2H), 1.48 (s, 2H).

### 2-(4-tert-butylphenyl)-4-{[3-(1,2,3,4-tetrahydroisoquinolin-2-yl)propyl]amino}-6H,7H-pyrazolo[1,5-a][1,3,5]triazin-7-one—Example 91

2-(4-tert-butylphenyl)-4-(methylsulfanyl)-6H,7H-pyrazolo[1,5-a][1,3,5]triazin-7-one (70 mg, 0.223 mmol) and 3-(1,2,3,4-tetrahydroisoquinolin-2-yl)propan-1-amine (108 mg, 0.445 mmol) were dissolved in Pyridine (1 mL). The reaction mixture was heated in a microwave at 140° C. for 2 h. The reaction mixture was evaporated to dryness and purified by preparative HPLC method A affording the title compound as a pale yellow oil (45 mg, 43%).

¹H NMR (500 MHz, Methanol-d4) δ 8.30 (s, 2H), 8.26 (d, J=8.6 Hz, 2H), 7.49 (d, J=8.6 Hz, 2H), 7.28-7.22 (m, 2H), 7.19 (t, J=6.7 Hz, 1H), 7.11 (d, J=7.5 Hz, 1H), 5.69 (s, 1H), 4.30 (s, 2H), 3.88 (t, J=6.5 Hz, 2H), 3.43 (t, J=6.3 Hz, 2H), 3.33 (m, 2H), 3.09 (t, J=6.3 Hz, 2H), 2.33-2.24 (m, 2H), 1.37 (s, 9H), LCMS Method B: rt 2.58 min, 99%; m/z 457.2 (MH⁺).

Examples 93 and 94 were synthesised from Compound BQ, which was synthesised from Example 92. The synthetic route for Compound BQ and subsequent reaction with amines is illustrated in Scheme 26.

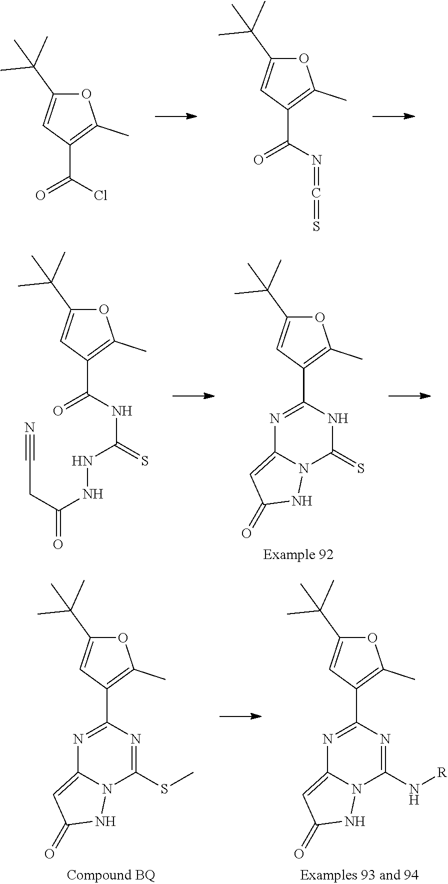

### 2-(5-tert-butyl-2-methylfuran-3-yl)-4-sulfanylidene-3H,4H,6H,7H-pyrazolo[1,5-a][1,3,5]triazin-7-one—Example 92

To a solution of sodium thiocyanate (818 mg, 9.96 mmol) in acetone (20 mL) was added 5-tert-butyl-2-methylfuran-3-carbonyl chloride (2.0 g, 9.96 mmol) reaction was stirred at RT for 1 h. 2-cyanoacetohydrazide (987 mg, 9.97 mmol) was added and reaction was stirred at 70° C. for 16 h. The reaction was cooled and concentrated to dryness, then dissolved in 20 mL of 5% aq. KOH and heated at 100° C. for 1 h. The reaction was cooled, acidified to pH 1 using 1M aq. HCl. The precipitate was isolated and purified by preparative HPLC method A affording the title compound as a white solid (110 mg, 5%).

¹H NMR (500 MHz, Methanol-d4) δ 6.63 (s, 1H), 5.82 (s, 1H), 2.63 (s, 3H), 1.32 (s, 9H), LCMS Method C: rt 1.37 min, 96%; m/z 305 (MH⁺).

### 2-(5-tert-butyl-2-methylfuran-3-yl)-4-(methylsulfanyl)-6H,7H-pyrazolo[1,5-a][1,3,5]triazin-7-one—Compound BQ

To an acetone (10 mL) suspension of 2-(5-tert-butyl-2-methylfuran-3-yl)-4-sulfanylidene-3H,4H,6H,7H-pyrazolo[1,5-a][1,3,5]triazin-7-one (110 mg, 0.35 mmol) was added K₂CO₃ (97 mg, 0.701 mmol) and iodomethane (24 μL, 0.386 mmol). The reaction was stirred at RT for 30 min. The reaction was concentrated, dissolved in water, acidified to pH 2 using 1M aq. HCl, and extracted with EtOAc. The combined organic extracts were dried over MgSO₄ and concentrated to give the title compound as a pale yellow solid (110 mg, 98%).

¹H NMR (250 MHz, Methanol-d4) δ 6.55 (s, 1H), 5.74 (s, 1H), 2.74 (s, 3H), 2.73 (s, 3H), 1.31 (s, 9H), LCMS Method C: rt 1.48 min, 100%; m/z 319 (MH⁺).

### 2-(5-tert-butyl-2-methylfuran-3-yl)-4-[(3-hydroxypropyl)amino]-6H,7H-pyrazolo[1,5-a][1,3,5]triazin-7-one—Example 93

2-(5-tert-butyl-2-methylfuran-3-yl)-4-(methylsulfanyl)-6H,7H-pyrazolo[1,5-a][1,3,5]triazin-7-one (25 mg, 0.079 mmol) and 3-aminopropan-1-ol (29.5 mg, 0.39 mmol) were dissolved in Pyridine (1 mL). The reaction mixture was heated in a microwave at 140° C. for 2 h. The reaction mixture was evaporated to dryness and purified by preparative HPLC method A affording the title compound as a colorless oil (5 mg, 17%).

¹H NMR (500 MHz, Methanol-d4) δ 6.53 (s, 1H), 5.59 (s, 1H), 3.78 (t, J=6.8 Hz, 2H), 3.73 (t, J=6.1 Hz, 2H), 2.75 (s, 3H), 1.98 (p, J=6.5 Hz, 2H), 1.33 (s, 9H). LCMS Method B: rt 2.95 min, 92%; m/z 346.1 (MH⁺).

### 2-(5-tert-butyl-2-methylfuran-3-yl)-4-(3-[(2,3-dihydro-1H-inden-2-yl)amino]propyl)amino)-6H,7H-pyrazolo[1,5-a][1,3,5]triazin-7-one—Example 94

2-(5-tert-butyl-2-methylfuran-3-yl)-4-(methylsulfanyl)-6H,7H-pyrazolo[1,5-a][1,3,5]triazin-7-one (25 mg, 0.079 mmol) and N-(3-aminopropyl)-2,3-dihydro-1H-inden-2-amine dihydrochloride (41 mg, 0.157 mmol) were dissolved in Pyridine (1 mL). The reaction mixture was heated in a microwave at 140° C. for 9 h. The reaction mixture was evaporated to dryness and purified by preparative HPLC method A affording the title compound as a pale beige solid (3 mg, 8%).

¹H NMR (500 MHz, Methanol-d4) δ 8.39 (s, 2H), 7.29-7.18 (m, 4H), 6.53 (s, 1H), 5.63 (s, 1H), 4.08 (p, J=7.6, 7.0 Hz, 1H), 3.80 (t, J=6.3 Hz, 2H), 3.39 (dd, J=16.3, 7.8 Hz, 2H), 3.26-3.19 (m, 2H), 3.07 (dd, J=16.3, 6.1 Hz, 2H), 2.71 (s, 3H), 2.17 (dt, J=14.1, 6.6 Hz, 2H), 1.32 (s, 9H). LCMS Method B: rt 2.64 min, 95%; m/z 461.2 (MH⁺).

Examples 95 to 97 were synthesised from Compound BS. The synthetic route for Compound BS and subsequent reaction with amines is illustrated in Scheme 27.

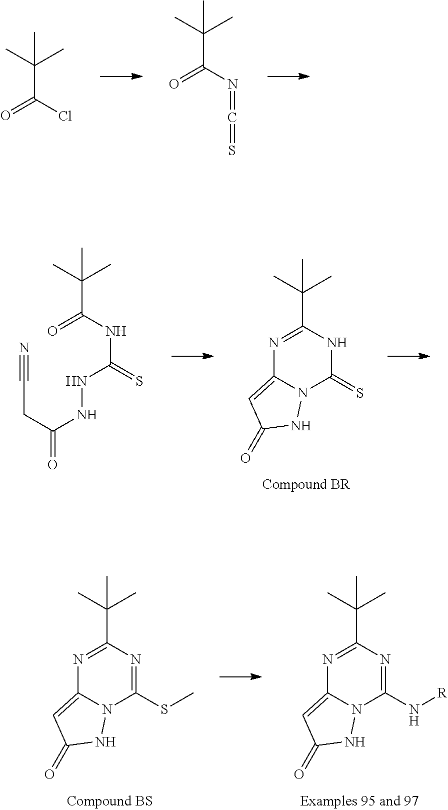

### 2-tert-butyl-4-sulfanylidene-3H,4H,6H,7H-pyrazolo[1,5-a][1,3,5]triazin-7-one—Compound BR

Sodium thiocyanate (681 mg, 8.29 mmol) was added to an acetonitrile (10 mL) solution of 2,2-dimethylpropanoyl chloride (1.0 g, 8.29 mmol). The mixture was stirred at RT for 2 h. 2-cyanoacetohydrazide (821.47 mg, 8.29 mmol) was added and mixture was stirred at 80° C. for 1 h. The reaction was concentrated to dryness, dissolved in 5% aq. KOH (10 mL) and ethanol (5 mL) and stirred at 100° C. for 1.5 h. Ethanol was removed under reduced pressure, the mixture was acidified to pH 1 using 1M aq. HCl and extracted with EtOAc. Combined organic fractions were dried over MgSO₄ and concentrated to give the title compound as a pale green solid (875 mg, 47%).

¹H NMR (500 MHz, Chloroform-d) δ 6.00 (s, 1H), 1.42 (s, 9H). LCMS Method C: rt 0.96 min, 100%; m/z 224.9 (MH⁺).

### 2-tert-butyl-4-(methylsulfanyl)-6H,7H-pyrazolo[1,5-a][1,3,5]triazin-7-one—Compound BS

To an acetone suspension of 2-tert-butyl-4-sulfanylidene-3H,4H,6H,7H-pyrazolo[1,5-a][1,3,5]triazin-7-one (0.88 g, 3.71 mmol) and K₂CO₃ (1.02 g, 7.41 mmol) was added iodomethane (254 μL, 4.08 mmol). Mixture was stirred at RT for 20 min. Acetone was removed under reduced pressure and the mixture was dissolved in Water, acidified and extracted with EtOAc/Et₂O/MeOH 2/1/0.1. Combined organics were dried over MgSO₄ and concentrated to give the title compound as a yellow solid. (0.81 g, 55%).

LCMS Method C: rt 1.22 min, 60%; m/z 238.9 (MH⁺).

### 2-tert-butyl-4-({3-[(2,3-dihydro-1H-inden-2-yl)amino]propyl}amino)-6H,7H-pyrazolo[1,5-a][1,3,5]triazin-7-one—Example 95

2-tert-butyl-4-(methylsulfanyl)-6H,7H-pyrazolo[1,5-a][1,3,5]triazin-7-one (75 mg, 0.189 mmol) and N-(3-aminopropyl)-2,3-dihydro-1H-inden-2-amine dihydrochloride (149 mg, 0.566 mmol) were dissolved in Pyridine (1 mL). The reaction mixture was heated in a microwave at 140° C. for 12 h. The reaction mixture was evaporated to dryness and purified by preparative HPLC method A affording the title compound as an orange oil (5 mg, 7%)

¹H NMR (500 MHz, Methanol-d4) δ 8.32 (s, 3H), 7.33-7.19 (m, 4H), 5.63 (s, 1H), 4.14-4.03 (m, 1H), 3.76 (t, J=6.4 Hz, 2H), 3.42 (dd, J=16.4, 7.8 Hz, 2H), 3.26-3.20 (m, 2H), 3.13 (dd, J=16.3, 6.0 Hz, 2H), 2.21-2.10 (m, 2H), 1.39 (s, 9H), LCMS Method B: rt 1.93 min, 99%; m/z 381.2 (MH⁺).

### 2-tert-butyl-4-(butylamino)-6H,7H-pyrazolo[1,5-a][1,3,5]triazin-7-one—Example 96

2-tert-butyl-4-(methylsulfanyl)-6H,7H-pyrazolo[1,5-a][1,3,5]triazin-7-one (75 mg, 0.189 mmol) and butan-1-amine (69 mg, 0.944 mmol) were dissolved in Pyridine (1 mL). The reaction mixture was heated in a microwave at 140° C. for 6 h. The reaction mixture was evaporated to dryness and purified by preparative HPLC method A affording the title compound as an orange oil (14 mg, 24%).

¹H NMR (500 MHz, Methanol-d4) δ 5.56 (s, 1H), 3.60 (t, J=7.1 Hz, 2H), 1.73-1.63 (m, 2H), 1.48-1.41 (m, 2H), 1.34 (s, 9H), 0.98 (t, J=7.4 Hz, 3H), LCMS Method B: rt 3.00 min, 100%; m/z 264.1 (MH⁺).

### 2-tert-butyl-4-{[3-(1,2,3,4-tetrahydroisoquinolin-2-yl)propyl]amino}-6H,7H-pyrazolo[1,5-a][1,3,5]triazin-7-one—Example 97

2-tert-butyl-4-(methylsulfanyl)-6H,7H-pyrazolo[1,5-a][1,3,5]triazin-7-one (40 mg, 0.101 mmol) and 3-(1,2,3,4-tetrahydroisoquinolin-2-yl)propan-1-amine (49 mg, 0.201 mmol) were dissolved in Pyridine (1 mL). The reaction mixture was heated in a microwave at 140° C. for 2 h. The reaction mixture was evaporated to dryness and purified by preparative HPLC method A affording the title compound as a yellow oil (11 mg, 28%).

¹H NMR (500 MHz, Methanol-d4) δ 8.32 (s, 2H), 7.33-7.21 (m, 3H), 7.17 (d, J=7.6 Hz, 1H), 5.61 (s, 1H), 4.35 (s, 2H), 3.77 (t, J=6.4 Hz, 2H), 3.49 (t, J=6.3 Hz, 2H), 3.32-3.28 (m, 2H), 3.16 (t, J=6.3 Hz, 2H), 2.30-2.19 (m, 2H), 1.37 (s, 9H), LCMS Method B: rt 1.81 min, 98%; m/z 381.2 (MH⁺).

Example 98 was synthesized by reacting Compound F with a commercially available amine.

### 4-({3-[benzyl(methyl)amino]propyl}amino)-2-(4-tert-butylphenyl)-6H,7H-pyrazolo[1,5-a][1,3,5]triazin-7-one—Example 98

2-tert-butyl-4-(methylsulfanyl)-6H,7H-pyrazolo[1,5-a][1,3,5]triazin-7-one (70 mg, 0.223 mmol) and (3-aminopropyl)(benzyl)methylamine (39.7 mg, 0.223 mmol) were dissolved in Pyridine (1 mL). The reaction mixture was heated in a microwave at 140° C. for 2 h. The reaction mixture was evaporated to dryness and purified by preparative HPLC method A affording the title compound (formate salt) as a yellow oil (50 mg, 50%).

¹H NMR (500 MHz, Methanol-d4) δ 8.39 (s, 2H), 8.31-8.26 (m, 2H), 7.56-7.49 (m, 2H), 7.41-7.26 (m, 5H), 5.72 (s, 1H), 4.22 (s, 2H), 3.84 (t, J=6.3 Hz, 2H), 3.23-3.15 (m, 2H), 2.77 (s, 3H), 2.27-2.16 (m, 2H), 1.40 (s, 9H), LCMS Method B: rt 2.58 min, 100%; m/z 446.2 (MH⁺).

Examples 100 to 102 were synthesised from Compound BT, which was synthesised from Example 99. The synthetic route for Compound BT and subsequent reaction with amines is illustrated in Scheme 28.

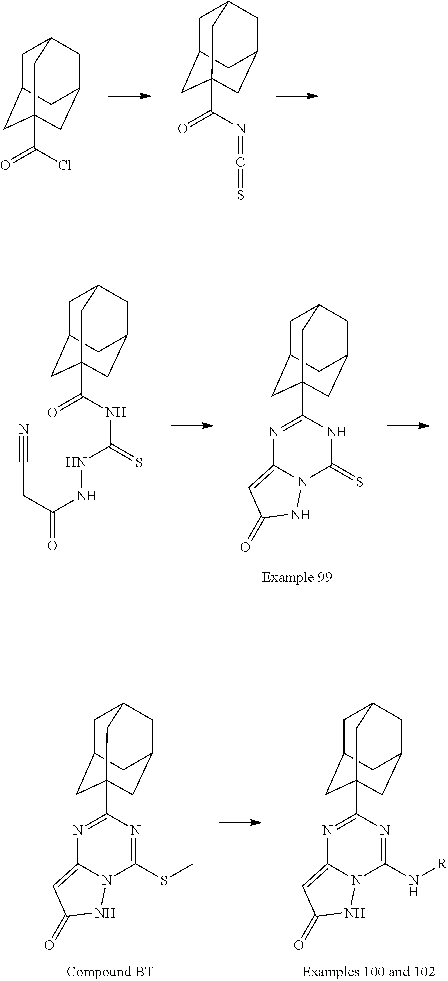

### 2-(adamantan-1-yl)-4-sulfanylidene-3H,4H,6H,7H-pyrazolo[1,5-a][1,3,5]triazin-7-one—Example 99

Sodium thiocyanate (1.24 g) was added to an acetone (25 mL) solution of adamantane-1-carbonyl chloride (3.00 g, 15.1 mmol). The reaction was stirred at RT for 1 h. 2-cyanoacetohydrazide (1.50 g, 15.1 mmol) was added and reaction was heated at 80° C. for 18 h. The mixture was filtered and the filtrate was concentrated, dissolved in 5% aq. KOH (50 mL) and ethanol (10 ml) and stirred at 105° C. for 30 min. The mixture was cooled to RT and concentrated, then acidified to pH 1 with 1M aq. HCl. The precipitate was collected by filtration, washed with water and dried overnight at 40° C. under vacuum to yield the title compound as a white solid (3.35 g, 80%).

¹H NMR (500 MHz, Methanol-d4) δ 5.83 (s, 1H), 2.11 (br s, 3H), 2.06 (d, J=3.0 Hz, 6H), 1.83 (t, J=2.9 Hz, 6H). LCMS Method F: rt 1.18 min, 89%; m/z 303.1 (MH⁺).

### 2-(adamantan-1-yl)-4-(methylsulfanyl)-6H,7H-pyrazolo[1,5-a][1,3,5]triazin-7-one—Compound BT

To an acetone suspension of 2-(adamantan-1-yl)-4-sulfanylidene-3H,4H,6H,7H-pyrazolo[1,5-a][1,3,5]triazin-7-one (0.25 g, 0.73 mmol) and K₂CO₃ (0.2 g, 1.45 mmol) was added iodomethane (114 mg, 0.80 mmol) and stirred at RT for 1 h. The reaction mixture was concentrated, dissolved in Water, acidified and extracted with EtOAc. The combined organics were dried over MgSO₄ and concentrated to yield the title product as a yellow solid. (0.16 g, 62%).

LCMS Method C: rt 1.37 min, 96%; m/z 316.9 (MH⁺).

### 2-(adamantan-1-yl)-4-[(3-hydroxypropyl)amino]-6H,7H-pyrazolo[1,5-a][1,3,5]triazin-7-one—Example 100

2-(adamantan-1-yl)-4-(methylsulfanyl)-6H,7H-pyrazolo[1,5-a][1,3,5]triazin-7-one (70 mg, 0.21 mmol) and 3-aminopropan-1-ol (79 mg, 1.06 mmol) were dissolved in Pyridine (1 mL). The reaction mixture was heated in a microwave at 140° C. for 1 h. The reaction mixture was evaporated to dryness and purified by preparative HPLC method A affording the title compound as a white solid (26 mg, 35%).

¹H NMR (500 MHz, Methanol-d4) δ 5.57 (s, 1H), 3.91-3.52 (m, 4H), 2.06 (d, J=7.1 Hz, 9H), 1.92 (p, J=6.5 Hz, 2H), 1.86-1.76 (m, 6H), LCMS Method B: rt 3.39 min, 100%; m/z 344.1 (MH⁺).

### 2-(adamantan-1-yl)-4-({3-[(2,3-dihydro-1H-inden-2-yl)amino]propyl}amino)-6H,7H-pyrazolo[1,5-a][1,3,5]triazin-7-one—Example 101

2-(adamantan-1-yl)-4-(methylsulfanyl)-6H,7H-pyrazolo[1,5-a][1,3,5]triazin-7-one (70 mg, 0.21 mmol), N-(3-aminopropyl)-2,3-dihydro-1H-inden-2-amine dihydrochloride (139.75 mg, 0.53 mmol) and trimethylamine (107 mg, 1.06 mmol) were dissolved in Pyridine (1 mL). The reaction mixture was heated in a microwave at 140° C. for 8 h. The reaction mixture was evaporated to dryness and purified by preparative HPLC method A affording the title compound as a white solid (12 mg, 12%).

¹H NMR (500 MHz, Methanol-d4) δ 8.30 (s, 2H), 7.32-7.20 (m, 4H), 5.63 (s, 1H), 4.10 (ddd, J=13.9, 7.6, 6.2 Hz, 1H), 3.77 (t, J=6.3 Hz, 2H), 3.42 (dd, J=16.4, 7.8 Hz, 2H), 3.25 (dd, J=9.5, 6.8 Hz, 2H), 3.13 (dd, J=16.3, 6.1 Hz, 2H), 2.20-2.08 (m, 10H), 1.91-1.75 (m, 7H). LCMS Method B: rt 3.20 min, 97%; m/z 459.1 (MH⁺).

### 2-(adamantan-1-yl)-4-{[3-(1,2,3,4-tetrahydroisoquinolin-2-yl)propyl]amino}-6H,7H-pyrazolo[1,5-a][1,3,5]triazin-7-one—Example 102

2-(adamantan-1-yl)-4-(methylsulfanyl)-6H,7H-pyrazolo[1,5-a][1,3,5]triazin-7-one (70 mg, 0.21 mmol), 3-(1,2,3,4-tetrahydroisoquinolin-2-yl)propan-1-amine (108 mg, 0.44 mmol) were dissolved in Pyridine (1 mL). The reaction mixture was heated in a microwave at 140° C. for 2 h. The reaction mixture was evaporated to dryness and purified by preparative HPLC method A affording the title compound as a white solid (20 mg, 19%).

¹H NMR (500 MHz, Methanol-d4) δ 8.32 (s, 2H), 7.31-7.21 (m, 3H), 7.16 (d, J=7.6 Hz, 1H), 5.61 (s, 1H), 4.34 (s, 2H), 3.78 (t, J=6.4 Hz, 2H), 3.48 (t, J=6.4 Hz, 2H), 3.32-3.26 (m, 2H), 3.16 (t, J=6.3 Hz, 2H), 2.25 (dt, J=14.4, 6.5 Hz, 2H), 2.08 (d, J=8.3 Hz, 9H), 1.82 (q, J=12.2 Hz, 6H), LCMS Method B: rt 3.39 min, 100%; m/z 459.2 (MH⁺).

Examples 103 and 104 were synthesised from Compound BV. Example 105 was synthesised from Compound BW. The synthetic route for Compound BV and Compound BW, and subsequent reactions with amines, is illustrated in Scheme 29.

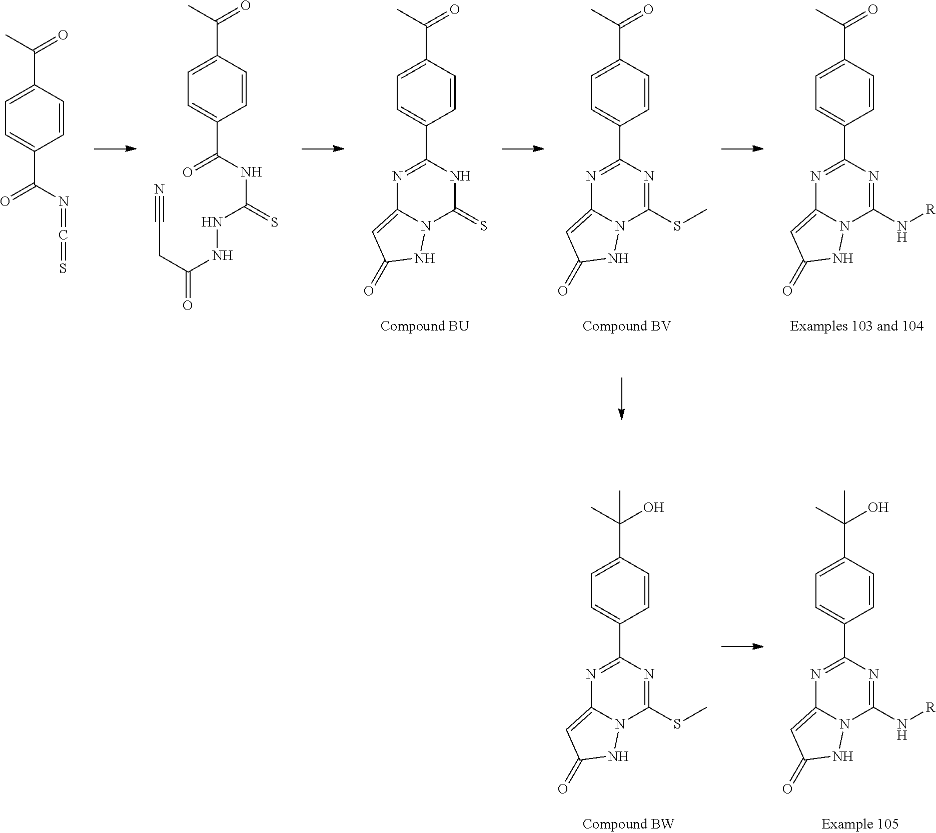

### 2-(4-acetylphenyl)-4-sulfanylidene-3H,4H,6H,7H-pyrazolo[1,5-a][1,3,5]triazin-7-one—Compound BU

Sodium thiocyanate (431 mg, 5.25 mmol) and 4-acetylbenzoyl chloride (960 mg, 5.25 mmol) were added to a solution of acetone (3 mL) and stirred at RT for 1 h. 2-cyanoacetohydrazide (520 mg, 5.25 mmol) was added and reaction was heated at 80° C. for 18 h. The mixture was cooled down, concentrated, and dissolved in 5% aq. KOH (15 mL), then stirred at 105° C. for 2 h. The mixture was cooled, concentrated, and acidified to pH 1 with 1M aq. HCl. The precipitate was collected by filtration, dissolved in MeCN/Toluene and concentrated under reduced pressure to give the title compound as a pale yellow solid (1.20 g, 61%).

¹H NMR (500 MHz, DMSO-d6) δ 13.83 (s, 1H), 11.85 (s, 1H), 8.22 (d, J=8.5 Hz, 2H), 8.10-8.07 (m, 2H), 6.09 (s, 1H), 2.65 (s, 3H), LCMS Method C: rt 0.87 min, 77%; m/z 286.9 (MH⁺).

### 2-(4-acetylphenyl)-4-(methylsulfanyl)-6H,7H-pyrazolo[1,5-a][1,3,5]triazin-7-one—Compound BV

To an acetone suspension of 2-(4-acetylphenyl)-4-sulfanylidene-3H,4H,6H,7H-pyrazolo[1,5-a][1,3,5]triazin-7-one (1.50 g, 3.85 mmol) and K₂CO₃ (1.17 g, 8 mmol) was added iodomethane (631 mg, 4.44 mmol). The mixture was stirred at RT for 90 mins, concentrated and dissolved in Water, acidified and extracted with EtOAc. Combined organics were dried over MgSO₄ and concentrated to give the title compound as a brown solid. (1.18 g, 77%).

¹H NMR (500 MHz, DMSO-d6) δ 11.81 (s, 1H), 8.55 (d, J=8.5 Hz, 2H), 8.11 (d, J=8.5 Hz, 2H), 6.04 (s, 1H), 2.82 (s, 3H), 2.65 (s, 3H), LCMS Method G: rt 1.68 min, 79%; m/z 300.9 (MH⁺).

### 2-(4-acetylphenyl)-4-({3-[(2,3-dihydro-1H-inden-2-yl)amino]propyl}amino)-6H,7H-pyrazolo[1,5-a][1,3,5]triazin-7-one—Example 103

2-(4-acetylphenyl)-4-(methylsulfanyl)-6H,7H-pyrazolo[1,5-a][1,3,5]triazin-7-one (50 mg, 0.13 mmol), N-(3-aminopropyl)-2,3-dihydro-1H-inden-2-amine dihydrochloride (70 mg, 0.26 mmol) and triethylamine (54 mg, 0.53 mmol) were dissolved in Pyridine (1 mL). The reaction mixture was heated at 80° C. for 24 h. The reaction mixture was evaporated to dryness and purified by preparative HPLC method A affording the title compound as a white solid (2.5 mg, 4%).

¹H NMR (500 MHz, Methanol-d4) δ 8.47 (d, J=8.5 Hz, 2H), 8.06 (d, J=8.6 Hz, 2H), 7.23-7.12 (m, 4H), 5.76 (s, 1H), 4.05 (t, J=6.3 Hz, 1H), 3.87 (t, J=6.4 Hz, 2H), 3.30 (dt, J=3.3, 1.6 Hz, 4H), 3.03 (dd, J=16.4, 6.0 Hz, 2H), 2.19 (dt, J=13.9, 6.9 Hz, 2H), LCMS Method B: rt 1.95 min, 91%; m/z 443.1 (MH⁺).

### 2-(4-acetylphenyl)-4-{[3-(4-chloro-3-methylphenoxy)propyl]amino}-6H,7H-pyrazolo[1,5-a][1,3,5]triazin-7-one—Example 104

2-(4-acetylphenyl)-4-(methylsulfanyl)-6H,7H-pyrazolo[1,5-a][1,3,5]triazin-7-one (80 mg, 0.213 mmol) and 4-(3-aminopropoxy)-1-chloro-2-methylbenzene (86 mg, 0.426 mmol) were dissolved in Pyridine (0.5 mL). The reaction mixture was heated at 70° C. for 24 h. The reaction mixture was evaporated to dryness and purified by preparative HPLC method A affording the title compound as a yellow solid (1.7 mg, 1.7%).

¹H NMR (500 MHz, Methanol-d4) δ 8.50 (s, 1H), 8.42-8.34 (m, 2H), 8.03-7.97 (m, 2H), 7.11 (d, J=8.7 Hz, 1H), 6.76 (d, J=3.0 Hz, 1H), 6.67 (dd, J=8.7, 3.0 Hz, 1H), 5.72 (s, 1H), 4.13 (t, J=5.7 Hz, 2H), 3.95 (t, J=6.4 Hz, 2H), 2.65 (s, 3H), 2.27-2.16 (m, 5H), LCMS Method B: rt 3.84 min, 99%; m/z 452.1 (MH⁺).

### 2-[4-(2-hydroxypropan-2-yl)phenyl]-4-(methylsulfanyl)-6H,7H-pyrazolo[1,5-a][1,3,5]triazin-7-one—Compound BW

To a solution of 2-(4-acetylphenyl)-4-(methylsulfanyl)-6H,7H-pyrazolo[1,5-a][1,3,5]triazin-7-one (80%, 200 mg, 0.533 mmol) in dry THF at 0° C., was added MeMgBr (1M in THF, 5.3 mL) and the reaction was warmed to RT and stirred for 1 h. The reaction was quenched with water, acidified with 1M aq. HCl and extracted with EtOAc. Combined organic fractions were dried over MgSO4, concentrated and purified by column chromatography (Biotage, 5 g SNAP KP-SIL, 15-80% EtOAc in Heptane 10 CV) to give the title compound as a white powder (169 mg, 79% purity, 25%).

¹H NMR (250 MHz, Methanol-d4) δ 8.44 (d, J=8.7 Hz, 2H), 7.65 (d, J=8.7 Hz, 2H), 5.88 (s, 1H), 2.84 (s, 3H), 1.60 (s, 6H), LCMS Method C: rt 1.13 min, 79%; m/z 316.9 (MH⁺).

### 4-({3-[(2,3-dihydro-1H-inden-2-yl)amino]propyl}amino)-2-[4-(2-hydroxypropan-2-yl)phenyl]-6H,7H-pyrazolo[1,5-a][1,3,5]triazin-7-one—Example 105

2-[4-(2-hydroxypropan-2-yl)phenyl]-4-(methylsulfanyl)-6H,7H-pyrazolo[1,5-a][1,3,5]triazin-7-one (79%, 55 mg, 0.13 mmol), N-(3-aminopropyl)-2,3-dihydro-1H-inden-2-amine dihydrochloride (70 mg, 0.26 mmol) and trimethylamine (54 mg, 0.533 mmol) were dissolved in Pyridine (0.5 mL). The reaction mixture was heated at 80° C. for 24 h. The reaction mixture was evaporated to dryness and purified by preparative HPLC method A affording the title compound as a white solid (4.3 mg, 7%).

¹H NMR (250 MHz, Methanol-d4) δ 8.39 (s, 2H), 8.29 (d, J=8.6 Hz, 2H), 7.59 (d, J=8.6 Hz, 2H), 7.23-7.12 (m, 5H), 5.70 (s, 1H), 4.08-3.94 (m, 1H), 3.86 (t, J=6.3 Hz, 2H), 3.30 (dt, J=3.3, 1.6 Hz, 4H), 3.00 (dd, J=16.3, 6.3 Hz, 2H), 2.15 (d, J=5.6 Hz, 2H), 1.56 (s, 6H), LCMS Method B: rt 1.86 min, 98%; m/z 459 (MH⁺).

Examples 107 and 108 were synthesised from Compound CB, which was synthesised from Example 106. The synthetic route for Compound CB and subsequent reaction with amines is illustrated in Scheme 30.

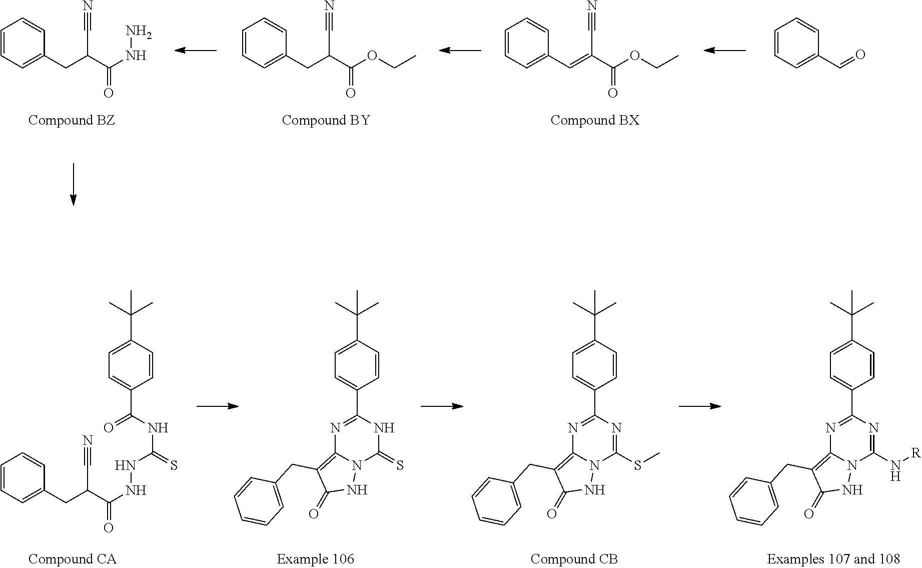

### Ethyl (2E)-2-cyano-3-phenylprop-2-enoate—Compound BX

Ethyl cyanoacetate (5.65 g, 50 mmol), benzaldehyde (5.3 g, 50 mmol) and piperidine (0.50 mL) were stirred in ethanol (15 ml) at RT for 16 h. Solid was filtered off, dissolved in methanol and the solution was concentrated to give the title compound as an oil that crystallized upon cooling (9.67 g, 87%).

¹H NMR (500 MHz, Chloroform-d) δ 8.25 (s, 1H), 8.04-7.93 (m, 2H), 7.55 (t, J=7.3 Hz, 1H), 7.51 (t, J=7.5 Hz, 2H), 4.39 (q, J=7.1 Hz, 2H), 1.40 (t, J=7.1 Hz, 3H).

### Ethyl 2-cyano-3-phenylpropanoate—Compound BY

Ethyl (2E)-2-cyano-3-phenylprop-2-enoate (5.0 g, 24.8 mmol) in Ethanol (100 mL) was hydrogenated at atmospheric pressure in the presence of Pd/C (125 mg, 10%) at RT for 16 h. The reaction mixture was purged with nitrogen, filtered through a pad of celite, washed with ethanol and the filtrate was concentrated to give the title compound as a pale yellow oil (5.05 g, 90%).

¹H NMR (500 MHz, Chloroform-d) δ 7.40-7.24 (m, 5H), 4.24 (q, J=7.2 Hz, 2H), 3.72 (dd, J=8.4, 5.8 Hz, 1H), 3.34-3.15 (m, 2H), 1.27 (t, J=7.1 Hz, 3H).

### 2-cyano-3-phenylpropanehydrazide—Compound BZ

To a solution of ethyl 2-cyano-3-phenylpropanoate (5.05 g, 24.85 mmol) in ethanol (100 mL) was added hydrazine hydrate (1:1) (1.33 mL). The reaction mixture was stirred at RT for 18 h. Hydrazine hydrate (1:1) (0.6 mL) was added and reaction mixture was stirred at RT for a further 16 h. The solution was concentrated, dissolved in MeOH and diluted with DCM/Pentane. The solution was concentrated under reduced pressure at 0° C. until precipitation occurred. The suspension that formed was then filtered off and washed with pentane to give the title compound as white crystals (3.10 g, 62%).

¹H NMR (500 MHz, DMSO-d6) 9.43 (s, 1H), 7.41-7.21 (m, 5H), 4.43 (s, 2H), 3.85 (dd, J=8.7, 6.9 Hz, 1H), 3.26-2.90 (m, 2H).

### N-({[(4-tert-butylphenyl)formamido]methanethioyl}amino)-2-cyano-3-phenylpropanamide—Compound CA

2-cyano-3-phenylpropanehydrazide (76 mg, 0.4 mmol) was added to suspension of 4-tert-butylbenzoyl isothiocyanate (88 mg, 0.4 mmol) in acetone (2 mL). The reaction was heated at 70° C. for 16 h, concentrated, dissolved in EtOAc and washed with water. The organic phase was concentrated to give crude title compound that was used directly in the next step.

### 8-benzyl-2-(4-tert-butylphenyl)-4-sulfanylidene-3H,4H,6H,7H-pyrazolo[1,5-a][1,3,5]triazin-7-one—Example 106

Crude N-({[(4-tert-butylphenyl)formamido]methanethioyl}amino)-2-cyano-3-phenylpropanamide (292 mg, 0.4 mmol) was dissolved in 5% aq. KOH (3 mL) and ethanol (1 mL) and stirred at 105° C. for 30 min. The reaction was cooled, ethanol was removed under reduced pressure and the mixture was acidified to pH1 with 1M aq. HCl. The suspension was centrifuged for 10 min and solid was collected to give the title compound as a white solid (185 mg, 98%).

¹H NMR (500 MHz, Methanol-d4) δ 8.03 (d, J=8.7 Hz, 2H), 7.61 (d, J=8.7 Hz, 2H), 7.38-7.27 (m, 2H), 7.24 (t, J=7.7 Hz, 2H), 7.14 (d, J=7.4 Hz, 1H), 3.91 (s, 2H), 1.38 (s, 9H). LCMS Method F: rt 4.65 min, 94%; m/z 391.1 (MH⁺).

### 8-benzyl-2-(4-tert-butylphenyl)-4-(methylsulfanyl)-6H,7H-pyrazolo[1,5-a][1,3,5]triazin-7-one—Compound CB

To an acetone (5 mL) suspension of 8-benzyl-2-(4-tert-butylphenyl)-4-sulfanylidene-3H,4H,6H,7H-pyrazolo[1,5-a][1,3,5]triazin-7-one (120 mg, 0.26 mmol) was added K₂CO₃ (70 mg, 0.51 mmol) and iodomethane (39 mg, 0.28 mmol). The reaction was stirred at RT until full conversion, concentrated, dissolved in water, acidified to pH1 with 1M aq. HCl and extracted with EtOAc. Combined organic layers were dried over MgSO₄ and concentrated to give the title compound as a white solid (70 mg, 58%).

¹H NMR (500 MHz, DMSO-d6) δ 11.84 (s, 1H), 8.38 (d, J=8.6 Hz, 2H), 7.59 (d, J=8.6 Hz, 2H), 7.33-7.25 (m, 4H), 7.17 (t, J=7.2 Hz, 1H), 3.92 (s, 2H), 2.81 (s, 3H), 1.35 (s, 9H). LCMS Method C: rt 1.36 min, 86%; m/z 405.0 (MH⁺).

### 8-benzyl-2-(4-tert-butylphenyl)-4-[(3-hydroxypropyl)amino]-6H,7H-pyrazolo[1,5-a][1,3,5]triazin-7-one—Example 107

8-benzyl-2-(4-tert-butylphenyl)-4-(methylsulfanyl)-6H,7H-pyrazolo[1,5-a][1,3,5]triazin-7-one (70 mg, 0.15 mmol) and 3-aminopropan-1-ol (56 mg, 0.74 mmol) were dissolved in Pyridine (1 mL). The reaction mixture was heated in a microwave at 140° C. for 1 h. The reaction mixture was evaporated to dryness and purified by preparative HPLC method A affording the title compound as a white solid (30 mg, 46%).

¹H NMR (500 MHz, Methanol-d4) δ 8.30 (d, J=8.5 Hz, 2H), 7.49 (d, J=8.5 Hz, 2H), 7.34 (d, J=7.6 Hz, 2H), 7.22 (t, J=7.7 Hz, 2H), 7.11 (t, J=7.4 Hz, 1H), 3.94 (s, 2H), 3.82 (t, J=6.7 Hz, 2H), 3.72 (t, J=6.1 Hz, 2H), 1.97 (p, J=6.5 Hz, 2H), 1.36 (s, 9H). LCMS Method F: rt 4.82 min, 100%; m/z 432.1 (MH⁺).

### 8-benzyl-2-(4-tert-butylphenyl)-4-({3-[(2,3-dihydro-1H-inden-2-yl)amino]propyl}amino)-6H,7H-pyrazolo[1,5-a][1,3,5]triazin-7-one—Example 108

8-benzyl-2-(4-tert-butylphenyl)-4-(methylsulfanyl)-6H,7H-pyrazolo[1,5-a][1,3,5]triazin-7-one (85 mg, 0.21 mmol), N-(3-aminopropyl)-2,3-dihydro-1H-inden-2-amine dihydrochloride (159 mg, 0.51 mmol) and trimethylamine (208 mg, 2.06 mmol) were dissolved in Pyridine (2 mL). The reaction mixture was heated in a microwave at 140° C. for 10 h. The reaction mixture was evaporated to dryness and purified by preparative HPLC method A affording the title compound as a brown solid (40 mg, 35%).

¹H NMR (500 MHz, Methanol-d4) δ 8.55 (s, 1H), 8.34-8.26 (m, 2H), 7.56-7.49 (m, 2H), 7.38 (d, J=7.1 Hz, 2H), 7.25 (t, J=7.7 Hz, 2H), 7.17 (s, 5H), 3.97 (m, 3H), 3.89 (t, J=6.4 Hz, 2H), 3.30-3.17 (m, 4H), 2.94 (dd, J=16.2, 6.4 Hz, 2H), 2.22-2.11 (m, 2H), 1.40 (s, 9H). LCMS Method B: rt 3.32 min, 100%; m/z 547.4 (MH⁺).

Examples 109 to 111 were synthesised from Compound CG. The synthetic route for Compound CG and subsequent reaction with amines is illustrated in Scheme 31.

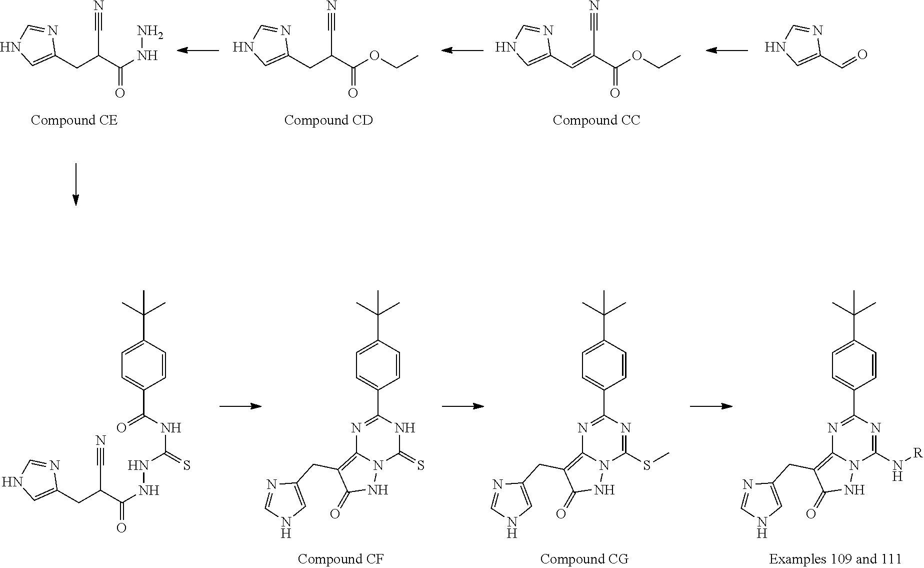

### Ethyl (2E)-2-cyano-3-(1H-imidazol-4-yl)prop-2-enoate—Compound CC

Ethyl cyanoacetate (1.13 g, 10 mmol), 1H-imidazole-4-carbaldehyde (0.96 g, 10 mmol) and piperidine (100 μL) were stirred in ethanol (5 ml) at RT for 18 h. The precipitate was filtered off and washed with Et₂O to give the title compound as a pale yellow powder (1.60 g, 86%).

¹H NMR (250 MHz, DMSO-d6) 12.94 (s, 1H), 8.19 (s, 1H), 8.10 (d, J=0.7 Hz, 1H), 8.01 (s, 1H), 4.28 (q, J=7.1 Hz, 2H), 1.30 (t, J=7.1 Hz, 3H). LCMS Method C: rt 0.88 min; m/z 191.9 (MH⁺).

### Ethyl 2-cyano-3-(1H-imidazol-4-yl)propanoate—Compound CD

Ethyl (2E)-2-cyano-3-(1H-imidazol-4-yl)prop-2-enoate (1.50 g, 7.84 mmol) in Ethanol (40 mL) was hydrogenated at atmospheric pressure in the presence of Pd/C (10%, 55 mg) at room temperature for 6 h. The reaction mixture was purged with nitrogen, filtered through a pad of celite, washed with ethanol, and the filtrate concentrated to give the title compound as a pale yellow oil (1.55 g, 92%).

¹H NMR (500 MHz, Chloroform-d) δ 7.58 (s, 1H), 6.97 (s, 1H), 4.26 (q, J=7.1 Hz, 2H), 3.94 (dd, J=8.4, 5.8 Hz, 1H), 3.29 (dd, J=14.6, 5.8 Hz, 1H), 3.20 (dd, J=14.6, 8.4 Hz, 1H), 1.29 (t, J=7.1 Hz, 3H).

### 2-cyano-3-(1H-imidazol-4-yl)propanehydrazide—Compound CE

To a solution of ethyl 2-cyano-3-(1H-imidazol-4-yl)propanoate (1.5 g, 6.98 mmol) in ethanol (45 mL) was added hydrazine hydrate (1:1) (374 μL, 7.68 mmol). The reaction mixture was stirred at RT for 92 h. Hydrazine hydrate (1:1) (280 μL, 5.76 mmol) was added and the reaction was stirred at RT for 18 h. The mixture was concentrated to dryness. The resulting crude material was triturated with MeOH/Et₂O/Pentane to give a white solid that was filtered off and washed with pentane, yielding the title compound as a white powder (0.84 g, 63%).

¹H NMR (500 MHz, DMSO-d6) δ 11.89 (s, 1H), 9.52 (s, 1H), 7.58 (s, 1H), 6.97 (s, 1H), 4.42 (s, 2H), 3.95-3.86 (m, 1H), 3.05-2.89 (m, 2H).

### 2-(4-tert-butylphenyl)-8-(1H-imidazol-4-ylmethyl)-4-sulfanylidene-3H,4H,6H,7H-pyrazolo[1,5-a][1,3,5]triazin-7-one—Compound CF

2-cyano-3-(1H-imidazol-4-yl)propanehydrazide (179 mg, 1 mmol) was added to a suspension of 4-tert-butylbenzoyl isothiocyanate (219 mg, 1 mmol) in acetonitrile (4 mL) and refluxed for 1 h. The reaction was concentrated to dryness. The crude was dissolved in 5% aq. KOH (5 mL) and ethanol (2 ml) and stirred at 105° C. for 15 min. The reaction was cooled to RT, ethanol was removed under reduce pressure and mixture was acidified to pH1 with 1M aq. HCl. A precipitate was filtered off to give the title compound as a white solid (0.37 g, 90% purity, 79%).

¹H NMR (250 MHz, DMSO-d6) δ 8.72 (s, 1H), 8.07 (d, J=8.6 Hz, 2H), 7.60-7.50 (m, 2H), 7.30 (s, 1H), 3.90 (s, 2H), 1.32 (s, 9H). LCMS Method C: rt 1.02 min; 90% m/z 380.9 (MH⁺).

### 2-(4-tert-butylphenyl)-8-(1H-imidazol-4-ylmethyl)-4-(methylsulfanyl)-6H,7H-pyrazolo[1,5-a][1,3,5]triazin-7-one—Compound CG

To an acetone (1 mL) suspension of 2-(4-tert-butylphenyl)-8-(1H-imidazol-4-ylmethyl)-4-sulfanylidene-3H,4H,6H,7H-pyrazolo[1,5-a][1,3,5]triazin-7-one (50 mg, 0.12 mmol) was added K₂CO₃ (33 mg, 0.24 mmol), iodomethane (19 mg, 0.13 mmol), and a few drops of DMF and the reaction was heated to 35° C. for 30 min. The mixture was concentrated to dryness, dissolved in water and extracted twice with EtOAc/MeOH (95/5 v/v). Combined organics were dried over MgSO₄ and concentrated to give a pale orange solid that was washed with Et₂O/pentane yielding the title compound as a pale orange solid (34 mg, 74%).

¹H NMR (500 MHz, Methanol-d4) δ 8.76 (s, 1H), 8.37 (d, J=8.1 Hz, 2H), 7.54 (d, J=8.1 Hz, 2H), 7.34 (s, 1H), 4.12 (s, 2H), 2.82 (s, 3H), 1.37 (s, 9H), LCMS Method C: rt 1.14 min; 100% m/z 395.0 (MH⁺).

### 2-(4-tert-butylphenyl)-4-({3-[(2,3-dihydro-1H-inden-2-yl)amino]propyl}amino)-8-(1H-imidazol-4-ylmethyl)-6H,7H-pyrazolo[1,5-a][1,3,5]triazin-7-one—Example 109

2-(4-tert-butylphenyl)-8-(1H-imidazol-4-ylmethyl)-4-(methylsulfanyl)-6H,7H-pyrazolo[1,5-a][1,3,5]triazin-7-one (54 mg, 0.11 mmol), N-(3-aminopropyl)-2,3-dihydro-1H-inden-2-amine dihydrochloride (79 mg, 0.26 mmol) and triethylamine (105 mg, 1.04 mmol) were dissolved in Pyridine (1 mL). The reaction mixture was heated in a microwave at 140° C. for 10 h. The reaction mixture was evaporated to dryness and purified by preparative HPLC method A affording the title compound as the tris formate sale as a pale yellow oil (11 mg, 19%).

¹H NMR (500 MHz, Methanol-d4) δ 8.34 (s, 3H), 8.32-8.25 (m, 3H), 7.54-7.48 (m, 2H), 7.18 (s, 4H), 7.12 (d, J=1.1 Hz, 1H), 4.07-3.97 (m, 3H), 3.88 (t, J=6.4 Hz, 2H), 3.27 (dt, J=16.5, 8.1 Hz, 4H), 3.02 (dd, J=16.2, 6.4 Hz, 2H), 2.20 (p, J=6.7 Hz, 2H), 1.38 (s, 9H). LCMS Method B: rt 1.97 min; 98% m/z 537.2 (MH⁺).

### 4-(butylamino)-2-(4-tert-butylphenyl)-8-(1H-imidazol-4-ylmethyl)-6H,7H-pyrazolo[1,5-a][1,3,5]triazin-7-one—Example 110

2-(4-tert-butylphenyl)-8-(1H-imidazol-4-ylmethyl)-4-(methylsulfanyl)-6H,7H-pyrazolo[1,5-a][1,3,5]triazin-7-one (50 mg, 0.10 mmol), and butan-1-amine (37 mg, 0.51 mmol) were dissolved in Pyridine (1 mL). The reaction mixture was heated in a microwave at 140° C. for 4 h. The reaction mixture was evaporated to dryness and purified by preparative HPLC method A affording the title compound as a pale yellow oil (11 mg, 43%).

¹H NMR (500 MHz, Methanol-d4) δ 8.38 (s, 1H), 8.37 (d, J=1.2 Hz, 1H), 8.33-8.26 (m, 2H), 7.55-7.48 (m, 2H), 7.18-7.12 (m, 1H), 4.04 (s, 2H), 3.74 (t, J=7.1 Hz, 2H), 1.77 (p, J=7.4 Hz, 2H), 1.55-1.45 (hex, J=7.4 Hz, 2H), 1.38 (s, 9H), 1.03 (t, J=7.4 Hz, 3H). LCMS Method B: rt 2.98 min; 99% m/z 420 (MH⁺).

### 2-(4-tert-butylphenyl)-8-(1H-imidazol-4-ylmethyl)-4-[(3-phenylpropyl)amino]-6H,7H-pyrazolo[1,5-a][1,3,5]triazin-7-one—Example 111

2-(4-tert-butylphenyl)-8-(1H-imidazol-4-ylmethyl)-4-(methylsulfanyl)-6H,7H-pyrazolo[1,5-a][1,3,5]triazin-7-one (50 mg, 0.10 mmol), and 3-phenylpropan-1-amine (69 mg, 0.51 mmol) were dissolved in Pyridine (1 mL). The reaction mixture was heated in a microwave at 140° C. for 2 h. The reaction mixture was evaporated to dryness and purified by preparative HPLC method A affording the title compound as a formate salt as a pale yellow oil (21 mg, 43%).

¹H NMR (500 MHz, Methanol-d4) δ 8.38 (s, 1H), 8.28-8.22 (m, 3H), 7.56-7.48 (m, 2H), 7.32-7.25 (m, 4H), 7.19 (m, 1H), 7.12 (s, 1H), 4.04 (s, 2H), 3.76 (t, J=7.2 Hz, 2H), 2.80 (t, J=7.5 Hz, 2H), 2.12 (p, J=7.4 Hz, 2H), 1.40 (s, 9H). LCMS Method B: rt 3.17 min; 100% m/z 482.1 (MH⁺).

Examples 113 to 117 were synthesised from Compound CJ, which was synthesised from Example 112. The synthetic route for Compound CJ and subsequent reaction with amines is illustrated in Scheme 32.

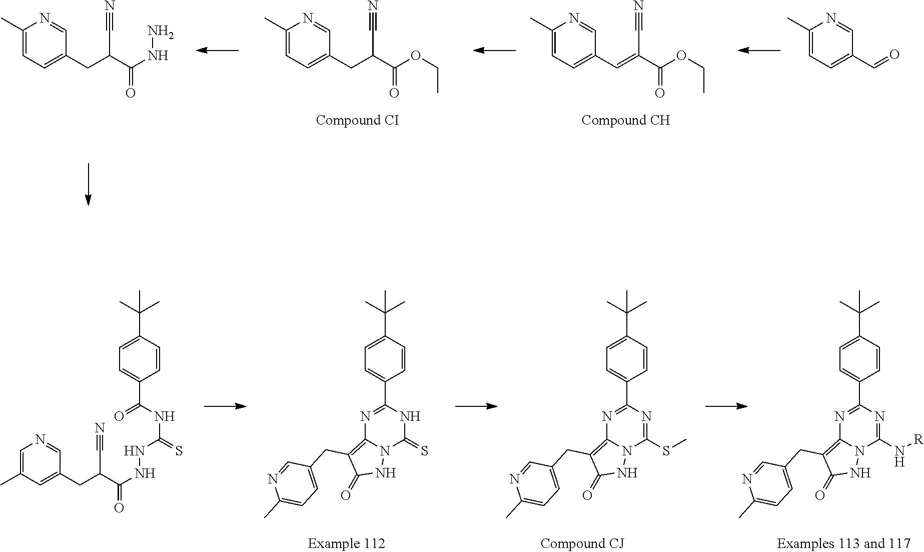

### Ethyl (2E)-2-cyano-3-(6-methylpyridin-3-yl)prop-2-enoate—Compound CH

Ethyl cyanoacetate (607 mg, 5.36 mmol), 6-methylpyridine-3-carbaldehyde (650 mg, 5.36 mmol), piperidine (0.26 mL) and acetic acid (48 mg) were stirred in ethanol (10 mL) at RT for 16 h. The mixture was diluted in TBME/Heptane and cooled on dry ice to induce precipitation. Solid formed was filtered off and washed with pentane to give the title compound as a white solid (0.89 g, 70%).

¹H NMR (500 MHz, Chloroformn-d) 8.78 (d, J=2.3 Hz, 1H), 8.51 (dd, J=8.3, 2.4 Hz, 1H), 8.23 (s, 1H), 7.32 (d, J=8.3 Hz, 1H), 4.40 (q, J=7.1 Hz, 2H), 2.65 (s, 3H), 1.40 (t, J=7.1 Hz, 3H). LCMS Method C: rt 1.08 min; 91% nm/z 216.9 (MH⁺).

### Ethyl 2-cyano-3-(6-methylpyridin-3-yl)propanoate—Compound CI

Ethyl (2E)-2-cyano-3-(6-methylpyridin-3-yl)prop-2-enoate (1.20 g, 5.55 mmol) in Ethanol (40 mL) was hydrogenated at atmospheric pressure in the presence of Pd/C (60 mg, 10%) at RT for 16 h. The reaction mixture was purged with nitrogen and filtered through a pad of celite, washing with methanol. The filtrate was concentrated to give the title compound as a pale yellow oil (1.21 g, 91%).

¹H NMR (500 MHz, Chloroform-d) 8.40 (d, J=2.2 Hz, 1H), 7.54 (dd, J=8.0, 2.4 Hz, 1H), 7.14 (d. J=8.0 Hz, 1H), 4.25 (q, J=7.2 Hz, 2H), 3.71 (dd, J=7.9, 5.9 Hz, 1H), 3.28-3.13 (m, 2H), 2.55 (s, 3H), 1.28 (t, J=7.1 Hz, 3H).

### 2-(4-tert-butylphenyl)-8-[(6-methylpyridin-3-yl)methyl]-4-sulfanylidene-3H,4H,6H,7H-pyrazolo[1,5-a][1,3,5]triazin-7-one—Example 112

To a solution of ethyl 2-cyano-3-(6-methylpyridin-3-yl)propanoate (1.1 g, 4.79 mmol) in ethanol (30 mL) was added hydrazine hydrate (1:1) (0.47 mL, 9.57 mmol). The reaction mixture was stirred at RT for 18 h. The mixture was concentrated and used directly in the next step. Crude 2-cyano-3-(6-methylpyridin-3-yl)propanehydrazide (0.98 g, 4.78 mmol) was added to a suspension of 4-tert-butylbenzoyl isothiocyanate (1.05 g, 4.78 mmol) in acetonitrile (15 mL). The mixture was heated at 80° C. for 1.5 h, then concentrated to dryness. The residue was dissolved in 5% aq. KOH (15 mL) and ethanol (5 ml) and the mixture was stirred at 105° C. for 30 mins. The reaction was cooled, ethanol was removed under reduced pressure and the mixture was acidified to pH1 with 1M aq. HCl. The precipitate was filtered off and purified by column chromatography (Biotage, 50 g SNAP KP-SIL, DCM/MeOH/AcOH, 90/10/1, 12 CV), affording the title compound as a pale beige solid (0.44 g, 29%).

¹H NMR (500 MHz, Methanol-d4) δ 8.42 (d, J=1.9 Hz, 1H), 8.02 (d, J=8.5 Hz, 2H), 7.70 (dd, J=8.0, 2.2 Hz, 1H), 7.60 (d, J=8.5 Hz, 2H), 7.21 (d, J=8.1 Hz, 1H), 3.91 (s, 2H), 2.47 (s, 3H), 1.37 (s, 9H). LCMS Method B: rt 2.25 min; 100% m/z 406.1 (MH⁺).

### 2-(4-tert-butylphenyl)-8-[(6-methylpyridin-3-yl)methyl]-4-(methylsulfanyl)-6H,7H-pyrazolo[1,5-a][1,3,5]triazin-7-one—Compound CJ

To an acetone (20 mL) suspension of 2-(4-tert-butylphenyl)-8-[(6-methylpyridin-3-yl)methyl]-4-sulfanylidene-3H,4H,6H,7H-pyrazolo[1,5-a][1,3,5]triazin-7-one (400 mg, 0.97 mmol) was added K₂CO₃ (273 mg, 1.97 mmol) and iodomethane (154 mg, 1.08 mmol) and the mixture was stirred at RT for 45 mins. Iodomethane (70 mg, 0.49 mmol) was added and the reaction was stirred for a further 45 mins. The mixture was concentrated, dissolved in water, acidified to pH1 with 1M aq. HCl, and extracted with EtOAc. The combined organic layers were dried over MgSO₄ and concentrated to give the title compound as a pale yellow solid (410 mg, 97%).

¹H NMR (500 MHz, DMSO-d6) δ 12.00 (s, 1H), 8.68 (s, 1H), 8.32 (d, J=8.5 Hz, 2H), 8.23 (d, J=6.8 Hz, 1H), 7.72 (d, J=8.0 Hz, 1H), 7.55 (d, J=8.5 Hz, 2H), 4.07 (s, 2H), 2.78 (s, 3H), 2.63 (s, 3H), 1.32 (s, 9H). LCMS Method C: rt 1.18 min; 97% m/z 420.0 (MH⁺).

### 2-(4-tert-butylphenyl)-4-[(3-hydroxypropyl)amino]-8-[(6-methylpyridin-3-yl)methyl]-6H,7H-pyrazolo[1,5-a][1,3,5]triazin-7-one—Example 113

2-(4-tert-butylphenyl)-8-[(6-methylpyridin-3-yl)methyl]-4-(methylsulfanyl)-6H,7H-pyrazolo[1,5-a][1,3,5]triazin-7-one (60 mg, 0.14 mmol), and 3-aminopropan-1-ol (43 mg, 0.57 mmol) were dissolved in Pyridine (1 mL). The reaction mixture was heated in a microwave at 140° C. for 2 h. The reaction mixture was evaporated to dryness and purified by preparative HPLC method B affording the title compound as a pale yellow solid (33 mg, 52%).

¹H NMR (500 MHz, Methanol-d4) δ 8.45 (d, J=1.9 Hz, 1H), 8.35-8.30 (m, 2H), 7.74 (dd, J=8.0, 2.2 Hz, 1H), 7.53 (d, J=8.5 Hz, 2H), 7.21 (d, J=8.0 Hz, 1H), 3.96 (s, 2H), 3.85 (t, J=6.8 Hz, 2H), 3.75 (t, J=6.1 Hz, 2H), 2.49 (s, 3H), 2.00 (p, J=6.5 Hz, 2H), 1.40 (s, 9H). LCMS Method B: rt 2.43 min; 99% m/z 447.1 (MH⁺).

### 2-(4-tert-butylphenyl)-4-{[3-(dimethylamino)propyl]amino}-8-[(6-methylpyridin-3-yl)methyl]-6H,7H-pyrazolo[1,5-a][1,3,5]triazin-7-one—Example 114

2-(4-tert-butylphenyl)-8-[(6-methylpyridin-3-yl)methyl]-4-(methylsulfanyl)-6H,7H-pyrazolo[1,5-a][1,3,5]triazin-7-one (60 mg, 0.14 mmol), and N,N-dimethylpropane-1,3-diamine (58 mg, 0.57 mmol) were dissolved in Pyridine (1 mL). The reaction mixture was heated in a microwave at 140° C. for 2 h. The reaction mixture was evaporated to dryness and purified by preparative HPLC method A affording the title compound formate salt as a white solid (38 mg, 56%).

¹H NMR (500 MHz, Methanol-d4) δ 8.52 (s, 1H), 8.43 (d, J=1.7 Hz, 1H), 8.31 (d, J=8.6 Hz, 2H), 7.72 (dd, J=8.0, 2.2 Hz, 1H), 7.53 (d, J=8.6 Hz, 2H), 7.19 (d, J=8.0 Hz, 1H), 3.94 (s, 2H), 3.83 (t, J=6.5 Hz, 2H), 3.21-3.15 (m, 2H), 2.78 (s, 6H), 2.47 (s, 3H), 2.21-2.13 (m, 2H), 1.38 (s, 9H). LCMS Method B: rt 1.79 min; 100% m/z 474.2 (MH⁺).

### 2-(4-tert-butylphenyl)-8-[(6-methylpyridin-3-yl)methyl]-4-[3-(1,2,3,4-tetrahydroisoquinolin-2-yl)propyl]amino-6H,7H-pyrazolo[1,5-a][1,3,5]triazin-7-one—Example 115

2-(4-tert-butylphenyl)-8-[(6-methylpyridin-3-yl)methyl]-4-(methylsulfanyl)-6H,7H-pyrazolo[1,5-a][1,3,5]triazin-7-one (60 mg, 0.14 mmol), and 3-(1,2,3,4-tetrahydroisoquinolin-2-yl)propan-1-amine (54 mg, 0.28 mmol) were dissolved in Pyridine (1 mL). The reaction mixture was heated in a microwave at 140° C. for 2 h. The reaction mixture was evaporated to dryness and purified by preparative HPLC method B affording the title compound as a yellow solid (18 mg, 22%).

¹H NMR (500 MHz, Methanol-d4) δ 8.46 (d, J=1.8 Hz, 1H), 8.32 (d, J=8.5 Hz, 2H), 7.74 (dd, J=8.0, 2.1 Hz, 1H), 7.48 (d, J=8.6 Hz, 2H), 7.20-7.08 (m, 4H), 7.01 (d, J=7.3 Hz, 1H), 3.94 (s, 2H), 3.82 (t, J=6.8 Hz, 2H), 3.73 (s, 2H), 2.94 (t, J=5.7 Hz, 2H), 2.85 (t, J=6.0 Hz, 2H), 2.78-2.68 (m, 2H), 2.48 (s, 3H), 2.10 (p, J=6.9 Hz, 2H), 1.39 (s, 9H).

LCMS Method B: rt 2.16 min; 100% m/z 562.2 (MH⁺).

### 2-(4-tert-butylphenyl)-4-({3-[(2,3-dihydro-1H-inden-2-yl)amino]propyl}amino)-8-[(6-methylpyridin-3-yl)methyl]-6H,7H-pyrazolo[1,5-a][1,3,5]triazin-7-one—Example 116

2-(4-tert-butylphenyl)-8-[(6-methylpyridin-3-yl)methyl]-4-(methylsulfanyl)-6H,7H-pyrazolo[1,5-a][1,3,5]triazin-7-one (60 mg, 0.14 mmol), N-(3-aminopropyl)-2,3-dihydro-1H-inden-2-amine dihydrochloride (75 mg, 0.28 mmol) and triethylamine (58 mg, 0.57 mmol) were dissolved in Pyridine (1 mL). The reaction mixture was heated in a microwave at 140° C. for 14 h. The reaction mixture was evaporated to dryness and purified by preparative HPLC method A affording the title compound as a yellow solid (15 mg, 18%).

¹H NMR (500 MHz, Methanol-d4) δ 8.46 (d, J=1.9 Hz, 1H), 8.25 (d, J=8.5 Hz, 2H), 7.75 (dd, J=8.0, 2.2 Hz, 1H), 7.49 (d, J=8.5 Hz, 2H), 7.18 (d, J=8.0 Hz, 1H), 7.13 (s, 4H), 3.94 (s, 2H), 3.86-3.80 (m, 3H), 3.20-3.15 (m, 2H), 3.08 (t, J=7.3 Hz, 2H), 2.90-2.79 (m, 2H), 2.47 (s, 3H), 2.11 (p, J=6.9 Hz, 2H), 1.38 (s, 9H). LCMS Method B: rt 2.20 min; 100% m/z 562.3 (MH⁺).

### 2-(4-tert-butylphenyl)-4-(4-methylpiperazin-1-yl)-8-[(6-methylpyridin-3-yl)methyl]-6H,7H-pyrazolo[1,5-a][1,3,5]triazin-7-one—Example 117

2-(4-tert-butylphenyl)-8-[(6-methylpyridin-3-yl)methyl]-4-(methylsulfanyl)-6H,7H-pyrazolo[1,5-a][1,3,5]triazin-7-one (60 mg, 0.14 mmol) and 1-methylpiperazine (43 mg, 0.43 mmol) were dissolved in Pyridine (1 mL). The reaction mixture was heated in a microwave at 140° C. for 10 h. The reaction mixture was evaporated to dryness and purified by preparative HPLC method A affording the title compound formate salt as a yellow solid (23 mg, 34%).

¹H NMR (500 MHz, Methanol-d4) δ 8.41 (d, J=1.9 Hz, 1H), 8.31 (s, 1H), 8.26 (d, J=8.5 Hz, 2H), 7.71 (dd, J=8.0, 2.2 Hz, 1H), 7.46 (d, J=8.6 Hz, 2H), 7.17 (d, J=8.0 Hz, 1H), 4.54 (s, 4H), 3.91 (s, 2H), 3.15 (t, J=4.8 Hz, 4H), 2.71 (s, 3H), 2.44 (s, 3H), 1.34 (s, 9H). LCMS Method B: rt 1.81 min; 100% m/z 472.2 (MH⁺).

Examples 119 and 120 were synthesised from Compound CL, which was synthesised from Example 118. The synthetic route for Compound CL and subsequent reaction with amines is illustrated in Scheme 33.

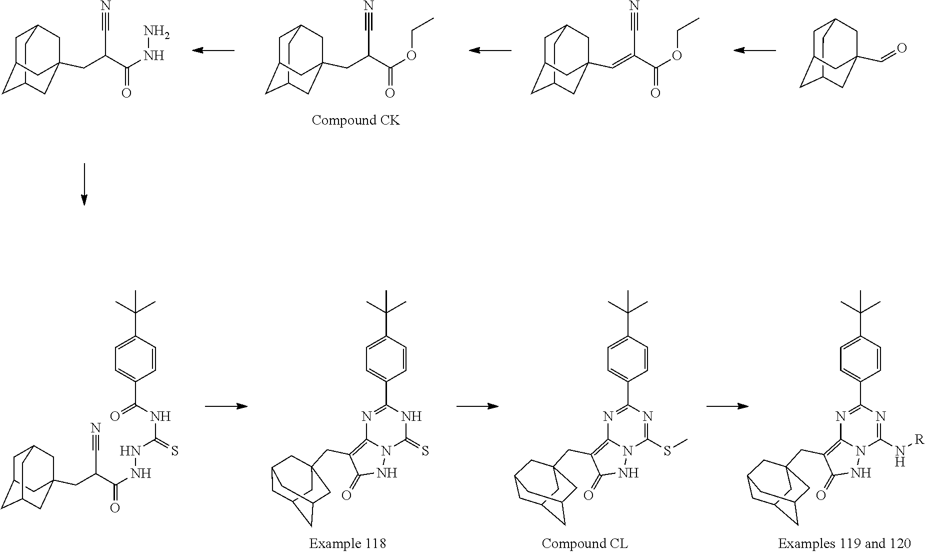

### Ethyl 3-(adamantan-1-yl)-2-cyanopropanoate—Compound CK

Ethyl 2-cyanoacetate (1.30 g, 11.6 mmol), adamantane-1-carbaldehyde (1.90 g, 11.6 mmol) and piperidine (0.12 mL) in EtOH (25 mL) were stirred at RT for 3 h. The reaction mixture was concentrated and used in the next step without further purification.

Ethyl (2E)-3-(adamantan-1-yl)-2-cyanoprop-2-enoate (3 g, 11.6 mmol) in Ethanol (40 mL) was hydrogenated at atmospheric pressure in the presence of Pd/C (75 mg, 10%) at RT for 6 h. The reaction mixture was purged with nitrogen, filtered through a pad of celite, washing with methanol, and the filtrate was concentrated to give the title compound as a pale yellow oil (2.85 g, 94%).

¹H NMR (500 MHz, Chloroform-d) δ 4.28 (q, J=7.1 Hz, 2H), 3.49 (dd, J=8.3, 4.8 Hz, 1H), 2.03 (s, 3H), 1.86-1.78 (m, 2H), 1.77-1.72 (m, 3H), 1.68-1.54 (m, 9H), 1.35 (t, J=7.1 Hz, 3H).

### 8-(adamantan-1-ylmethyl)-2-(4-tert-butylphenyl)-4-sulfanylidene-3H,4H,6H,7H-pyrazolo[1,5-a][1,3,5]triazin-7-one—Example 118

To a solution of ethyl 3-(adamantan-1-yl)-2-cyanopropanoate (3.00 g, 11.5 mmol) in ethanol (50 mL) was added hydrazine hydrate (1:1) (0.80 mL, 16.4 mmol). The reaction mixture was stirred at RT for 72 h. The mixture was concentrated to give a crude product, which was dissolved in DCM/Et₂O/pentane and slowly concentrated at low temperature. The resulting precipitate was filtered off and washed with pentane to give the title compound as a white solid, which was directly used in the next step.

Crude 3-(adamantan-1-yl)-2-cyanopropanehydrazide (1.25 g, 5.05 mmol) was added to a suspension of 4-tert-butylbenzoyl isothiocyanate (1.10 g, 5.05 mmol) in acetonitrile and mixture was heated at 80° C. for 1.5 h. The reaction was concentrated to dryness, diluted in 5% aq. KOH (15 mL) and ethanol (5 ml) and stirred at 105° C. for 1 h. The reaction was cooled to RT, ethanol was removed under reduced pressure and the mixture was acidified to pH1 with 1M aq. HCl. The precipitate was triturated with DCM/pentane and filtered off to give the title compound as a white solid (0.55 g, 23%).

¹H NMR (500 MHz, Chloroform-d) δ 9.83 (s, 1H), 7.94 (d, J=8.4 Hz, 2H), 7.59 (d, J=8.5 Hz, 2H), 2.43 (s, 2H), 1.95 (s, 3H), 1.71-1.55 (m, 12H), 1.37 (s, 9H). LCMS Method C: rt 1.58 min; 98% m/z 449.1 (MH⁺).

### 8-(adamantan-1-ylmethyl)-2-(4-tert-butylphenyl)-4-(methylsulfanyl)-6H,7H-pyrazolo[1,5-a][1,3,5]triazin-7-one—Compound CL

To an acetone (30 mL) suspension of 8-(adamantan-1-ylmethyl)-2-(4-tert-butylphenyl)-4-sulfanylidene-3H,4H,6H,7H-pyrazolo[1,5-a][1,3,5]triazin-7-one (1450 mg, 3.10 mmol) was added K₂CO₃ (858 mg, 6.21 mmol) and iodomethane (528 mg, 3.72 mmol). The reaction was stirred at RT for 4 h. The mixture was concentrated, dissolved in water, acidified to pH1 with 1M aq. HCl and extracted with EtOAc. The combined organic layers were dried over MgSO₄ and concentrated to give the title compound as a pale yellow solid (1.04 g, 69%).

¹H NMR (500 MHz, DMSO-d6) δ 11.59 (s, 1H), 8.35 (d, J=8.5 Hz, 2H), 7.57 (d, J=8.5 Hz, 2H), 2.79 (s, 3H), 2.32 (s, 2H), 1.89 (s, 3H), 1.65-1.59 (m, 3H), 1.59-1.49 (m, 9H), 1.33 (s, 9H).

### 8-(adamantan-1-ylmethyl)-2-(4-tert-butylphenyl)-4-[(3-hydroxypropyl)amino]-6H,7H-pyrazolo[1,5-a][1,3,5]triazin-7-one—Example 119

8-(adamantan-1-ylmethyl)-2-(4-tert-butylphenyl)-4-(methylsulfanyl)-6H,7H-pyrazolo[1,5-a][1,3,5]triazin-7-one (100 mg, 0.22 mmol), and 3-aminopropan-1-ol (65 mg, 0.87 mmol) were dissolved in Pyridine (1.5 mL). The reaction mixture was heated in a microwave at 140° C. for 2 h. The reaction mixture was evaporated to dryness and dissolved in DCM/Et₂O. The precipitate was filtered and purified by column chromatography (SiO₂, DCM/7N NH₃ in MeOH 90/10), to give a pale yellow solid that was further purified by preparative HPLC method B affording the title compound as a pale yellow solid (19 mg, 18%).

¹H NMR (500 MHz, Methanol-d4) δ 8.32-8.26 (m, 2H), 7.52-7.46 (m, 2H), 3.83 (t, J=6.7 Hz, 2H), 3.73 (t, J=6.1 Hz, 2H), 2.36 (s, 2H), 1.98 (p, J=6.5 Hz, 2H), 1.93 (s, 3H), 1.74-1.67 (m, 3H), 1.67-1.59 (m, 9H), 1.37 (s, 9H). LCMS Method B: rt 5.38 min; 98% m/z 490.2 (MH⁺).

### 8-(adamantan-1-ylmethyl)-2-(4-tert-butylphenyl)-4-{[3-(dimethylamino)propyl]amino}-6H,7H-pyrazolo[1,5-a][1,3,5]triazin-7-one—Example 120

8-(adamantan-1-ylmethyl)-2-(4-tert-butylphenyl)-4-(methylsulfanyl)-6H,7H-pyrazolo[1,5-a][1,3,5]triazin-7-one (100 mg, 0.22 mmol), and (3-aminopropyl)dimethylamine (88 mg 0.87 mmol) were dissolved in Pyridine (1.5 mL). The reaction mixture was heated in a microwave at 140° C. for 2 h. The reaction mixture was evaporated to dryness and purified by column chromatography (SiO₂, DCM/MeOH/Et₃N 90/10/1 then DCM/7N NH₃ in MeOH 90/10) to give the title compound as a white solid (45 mg, 39%).

¹H NMR (500 MHz, Chloroform-d) δ 8.29 (d, J=8.6 Hz, 2H), 7.40 (d, J=8.6 Hz, 2H), 6.74 (s, 1H), 3.77 (t, J=6.3 Hz, 2H), 2.61 (t, J=6.6 Hz, 2H), 2.34 (s, 6H), 2.29 (s, 2H), 1.98 (p, J=6.3 Hz, 2H), 1.83 (s, 3H), 1.62-1.45 (m, 12H), 1.28 (s, 9H). LCMS Method B: rt 3.85 min; 98% m/z 517.2 (MH⁺).

Examples 122 to 125 were synthesised from Compound CM, which was synthesised from Example 121. The synthetic route for Compound CM and subsequent reaction with amines is illustrated in Scheme 34.

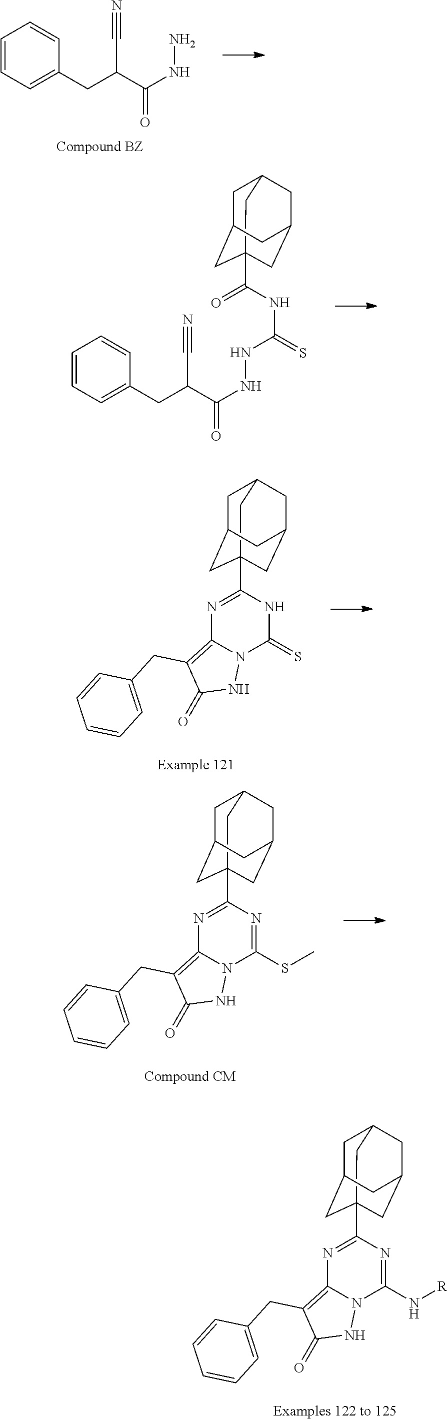

### 2-(adamantan-1-yl)-8-benzyl-4-sulfanylidene-3H,4H,6H,7H-pyrazolo[1,5-a][1,3,5]triazin-7-one—Example 121

2-cyano-3-phenylpropanehydrazide (598 mg, 3.16 mmol) was added to a suspension of adamantane-1-carbonyl isothiocyanate (700 mg, 3.16 mmol) in acetonitrile (15 mL) and heated at 80° C. for 1.5 h. The reaction was concentrated to dryness, diluted in 5% aq. KOH (15 mL) and ethanol (5 ml) and stirred at 105° C. for 0.5 h. The reaction was cooled, ethanol was removed under reduced pressure and the mixture was acidified to pH1 with 1M aq. HCl. The precipitate was filtered off and washed with water to give the title compound as white solid (1.19 g, 94%).

¹H NMR (500 MHz, DMSO-d6) δ 12.83 (s, 1H), 11.92 (s, 1H), 7.29-7.22 (m, 4H), 7.16 (m, 1H), 3.76 (s, 2H), 2.05 (s, 9H), 1.71 (d, J=15.3 Hz, 6H). LCMS Method B: rt 4.31 min; 96% m/z 393.1 (MH⁺).

### 2-(adamantan-1-yl)-8-benzyl-4-(methylsulfanyl)-6H,7H-pyrazolo[1,5-a][1,3,5]triazin-7-one—Compound CM

To an acetone (40 mL) suspension of 2-(adamantan-1-yl)-8-benzyl-4-sulfanylidene-3H,4H,6H,7H-pyrazolo[1,5-a][1,3,5]triazin-7-one (1190 mg, 2.91 mmol) was added K₂CO₃ (804 mg, 5.82 mmol) and iodomethane (496 mg, 3.49 mmol) and the reaction was stirred at RT for 3 h. The mixture was concentrated, dissolved in water, acidified to pH1 with 1M aq. HCl and extracted with EtOAc. The combined organic layers were dried over MgSO₄ and concentrated to give the title compound as a pale yellow solid (1.01 g, 82%).

¹H NMR (500 MHz, DMSO-d6) δ 11.66 (s, 1H), 7.25-7.08 (m, 5H), 3.79 (s, 2H), 2.65 (s, 3H), 2.08-1.97 (m, 9H), 1.76-1.69 (m, 6H). LCMS Method C: rt 1.61 min; 97% m/z 407.0 (MH⁺).

### 2-(adamantan-1-yl)-8-benzyl-4-[(3-hydroxypropyl)amino]-6H,7H-pyrazolo[1,5-a][1,3,5]triazin-7-one—Example 122

2-(adamantan-1-yl)-8-benzyl-4-(methylsulfanyl)-6H,7H-pyrazolo[1,5-a][1,3,5]triazin-7-one (60 mg, 0.15 mmol) and 3-aminopropan-1-ol (44 mg, 0.59 mmol) were dissolved in Pyridine (1 mL). The reaction mixture was heated in a microwave at 140° C. for 2 h. The reaction mixture was evaporated to dryness and purified by preparative HPLC method C affording the title compound as a colourless oil (7 mg, 10%).

¹H NMR (500 MHz, Methanol-d4) δ 7.31 (d, J=7.5 Hz, 2H), 7.21 (t, J=7.6 Hz, 2H), 7.11 (t, J=7.3 Hz, 1H), 3.88 (s, 2H), 3.70 (q, J=6.6 Hz, 4H), 2.08 (s, 9H), 1.92 (p, J=6.5 Hz, 2H), 1.82 (s, 6H). LCMS Method B: rt 4.43 min; 100% m/z 434.2 (MH⁺).

### 2-(adamantan-1-yl)-8-benzyl-4-{[3-(dimethylamino)propyl]amino}-6H,7H-pyrazolo[1,5-a][1,3,5]triazin-7-one—Example 123

2-(adamantan-1-yl)-8-benzyl-4-(methylsulfanyl)-6H,7H-pyrazolo[1,5-a][1,3,5]triazin-7-one (60 mg, 0.15 mmol) and N,N-dimethylpropane-1,3-diamine (60 mg, 0.59 mmol) were dissolved in Pyridine (1 mL). The reaction mixture was heated in a microwave at 140° C. for 2 h, evaporated to dryness and purified by preparative HPLC method A affording the title compound as a yellow oil (7 mg, 10%).

¹H NMR (500 MHz, DMSO-d6) δ 7.90 (t, J=5.4 Hz, 1H), 7.25 (dt, J=15.1, 7.5 Hz, 4H), 7.13 (t, J=7.1 Hz, 1H), 3.78 (s, 2H), 3.51 (p, J=6.5 Hz, 2H), 2.29 (t, J=6.8 Hz, 2H), 2.15 (s, 6H), 2.05 (s, 3H), 1.99 (s, 6H), 1.79-1.67 (m, 8H). LCMS Method B: rt 3.12 min; 95% m/z 461.2 (MH⁺).

### 2-(adamantan-1-yl)-8-benzyl-4-{[3-(1,2,3,4-tetrahydroisoquinolin-2-yl)propyl]amino}-6H,7H-pyrazolo[1,5-a][1,3,5]triazin-7-one—Example 124

2-(adamantan-1-yl)-8-benzyl-4-(methylsulfanyl)-6H,7H-pyrazolo[1,5-a][1,3,5]triazin-7-one (60 mg, 0.15 mmol) and 3-(1,2,3,4-tetrahydroisoquinolin-2-yl)propan-1-amine (56 mg, 0.29 mmol) were dissolved in Pyridine (1 mL). The reaction mixture was heated in a microwave at 140° C. for 6 h, evaporated to dryness and purified by preparative HPLC method A affording the title compound as a white solid (30 mg, 37%).

¹H NMR (500 MHz, Methanol-d4) δ 8.43 (s, 1H), 7.32 (d, J=7.2 Hz, 2H), 7.28-7.16 (m, 5H), 7.16-7.09 (m, 2H), 4.17 (s, 2H), 3.89 (s, 2H), 3.75 (t, J=6.5 Hz, 2H), 3.31 (t, J=6.3 Hz, 2H), 3.18-3.05 (m, 4H), 2.19 (dt, J=14.4, 6.5 Hz, 2H), 2.09 (s, 9H), 1.89-1.76 (m, 6H). LCMS Method B: rt 3.47 min; 100% m/z 549.2 (MH⁺).

### 2-(adamantan-1-yl)-8-benzyl-4-({3-[(2,3-dihydro-1H-inden-2-yl)amino]propyl}amino)-6H,7H-pyrazolo[1,5-a][1,3,5]triazin-7-one—Example 125

2-(adamantan-1-yl)-8-benzyl-4-(methylsulfanyl)-6H,7H-pyrazolo[1,5-a][1,3,5]triazin-7-one (60 mg, 0.15 mmol), N-(3-aminopropyl)-2,3-dihydro-1H-inden-2-amine dihydrochloride (78 mg, 0.29 mmol) and trimethylamine (60 mg, 0.59 mmol) were dissolved in Pyridine (1 mL). The reaction mixture was heated in a microwave at 140° C. for 14 h, evaporated to dryness and purified by preparative HPLC method A affording the title compound formate salt as a brown solid (17 mg, 21%).

¹H NMR (500 MHz, Methanol-d4) δ 8.43 (s, 1H), 7.32 (d, J=7.2 Hz, 2H), 7.29-7.19 (m, 6H), 7.12 (t, J=7.3 Hz, 1H), 4.11-4.03 (m, 1H), 3.89 (s, 2H), 3.75 (t, J=6.3 Hz, 2H), 3.40 (dd, J=16.3, 7.8 Hz, 2H), 3.26-3.19 (m, 2H), 3.11 (dd, J=16.3, 6.1 Hz, 2H), 2.19-2.06 (m, 11H), 1.90-1.78 (m, 6H). LCMS Method B: rt 3.53 min; 100% m/z 549.2 (MH⁺).

Example 126 was synthesised by reacting Compound CN with Compound E. Compound CN was synthesised in 4 steps from 4-(carbonochloridoyl)pyridin-1-ium-1-olate in a manner analogous to that for Compound BQ (see Scheme 26). The reaction between Compound CN and Compound E is illustrated in Scheme 35

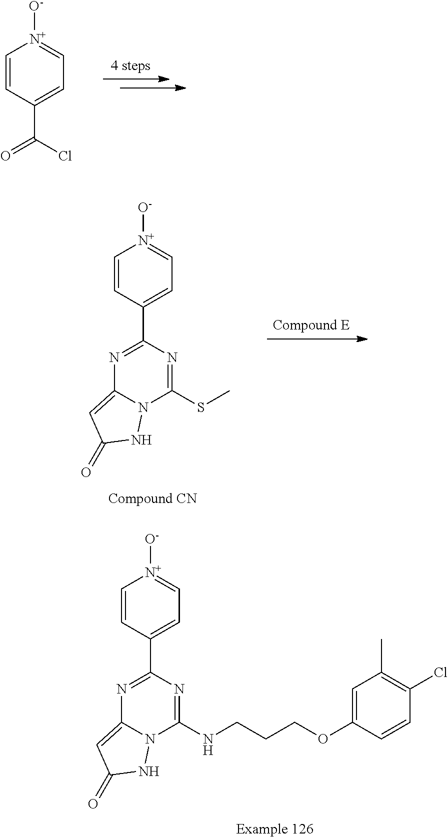 [SMILES: O=C(Cl)c1cc[n+]([O-])cc1 ; CSc1nc(-c2cc[n+]([O-])cc2)nc2cc(=O)[nH]n12 ; Cc1cc(OCCCNc2nc(-c3cc[n+]([O-])cc3)nc3cc(=O)[nH]n23)ccc1Cl]

### 4-(4-{[3-(4-chloro-3-methylphenoxy)propyl]amino}-7-oxo-6H,7H-pyrazolo[1,5-a][1,3,5]triazin-2-yl)pyridin-1-ium-1-olate—Example 126

4-[4-(methylsulfanyl)-7-oxo-6H,7H-pyrazolo[1,5-a][1,3,5]triazin-2-yl]pyridin-1-ium-1-olate (40 mg, 0.15 mmol) and 4-(3-aminopropoxy)-1-chloro-2-methylbenzene (93.35 μl, 0.36 mmol) were dissolved in Pyridine (1 mL). The reaction mixture was heated in a microwave at 140° C. for 6 h, evaporated to dryness and purified by preparative HPLC method A affording the title compound as a pale yellow solid (4.0 mg, 6%).

¹H NMR (500 MHz, Methanol-d4) δ 8.34 (s, 4H), 7.11 (d, J=8.7 Hz, 1H), 6.72 (d, J=2.9 Hz, 1H), 6.64 (dd, J=8.7, 3.0 Hz, 1H), 5.76 (s, 1H), 4.14 (t, J=5.6 Hz, 2H), 3.95 (t, J=6.3 Hz, 2H), 2.26-2.18 (m, 5H). LCMS Method B: rt 2.91 min; 100% m/z 427.1 (MH⁺).

### Biological Assays

The following PGT enzymes were expressed and purified as previously described (Wang et al., J. Am. Chem. Soc. 2011, 133, 8528-8530; Heaslet et al., J. Struct. Biol. 2009, 167, 129-135; Wang et al., J. Am. Chem. Soc. 2008, 130, 14068-14069). All of the references are incorporated by reference herein.
SgtB ΔTM of S. aureus (referred to as “SgtB”),
PBP2a (A68-N728) of E. faecalis (referred to as “PBP2a”)

Probe compound CMG121 was prepared as previously described (Gampe et al., J. Am. Chem. Soc. 2013, 135 (10), 3776-3779; U.S. Provisional Patent Application Nos. 61/621,229 filed on Apr. 6, 2012).

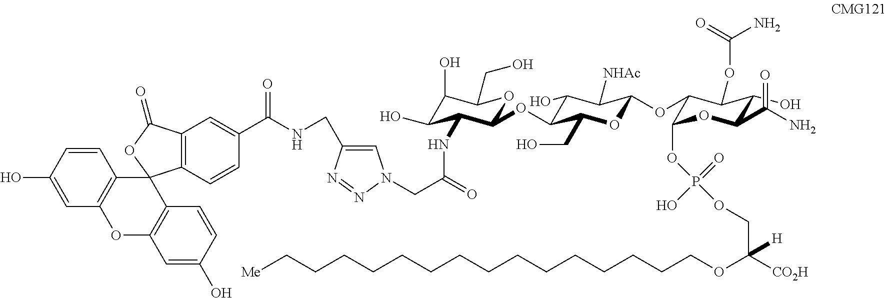

The S. aureus strain used in the reporter gene assay (S. aureus RN4220 pXEN-P_{cwrA}-lux) was prepared as previously described (Balibar et al., Microbiology, 2010, 156, 1372-1383).

I. Dose-Dependent Displacement of CMG121 from S. aureus SgtB by Fluorescence Polarization

A black 384 well plate (Corning NBS Low Volume No. 3820) was filled (10 μL per well) with an equilibrated solution containing 10 mM TRIS (pH=8.0), 100 mM NaCl, 75 nM CMG121, and 1.0-1.5 μM S. aureus SgtB (depending on protein batch). Due to presence of 8 mM CHAPS detergent in the buffer of the S. aureus SgtB stock, the final assay solution contains 100-160 μM CHAPS. Using an HP D300 Digital Dispenser, for each compound a 1/1 dilution series (12 wells) of the primary hit compound in DMSO (normalized to 1 μL with DMSO) was prepared and added to the aforementioned assay solution. The plate was incubated at 4° C. for 30 min and read with a Perkin Elmer EnVision microplate reader (FP-read out, excitation: 480 nm; emission: 535 nm). Data was plotted for fluorescence polarization (FP) (y-axis) vs. log(concentration of test compound in μM; x-axis), and IC₅₀ values were determined by non-linear regression analysis using GraphPad Prism 5.0 (GraphPad Software, Inc.; La Jolla, Calif., USA).

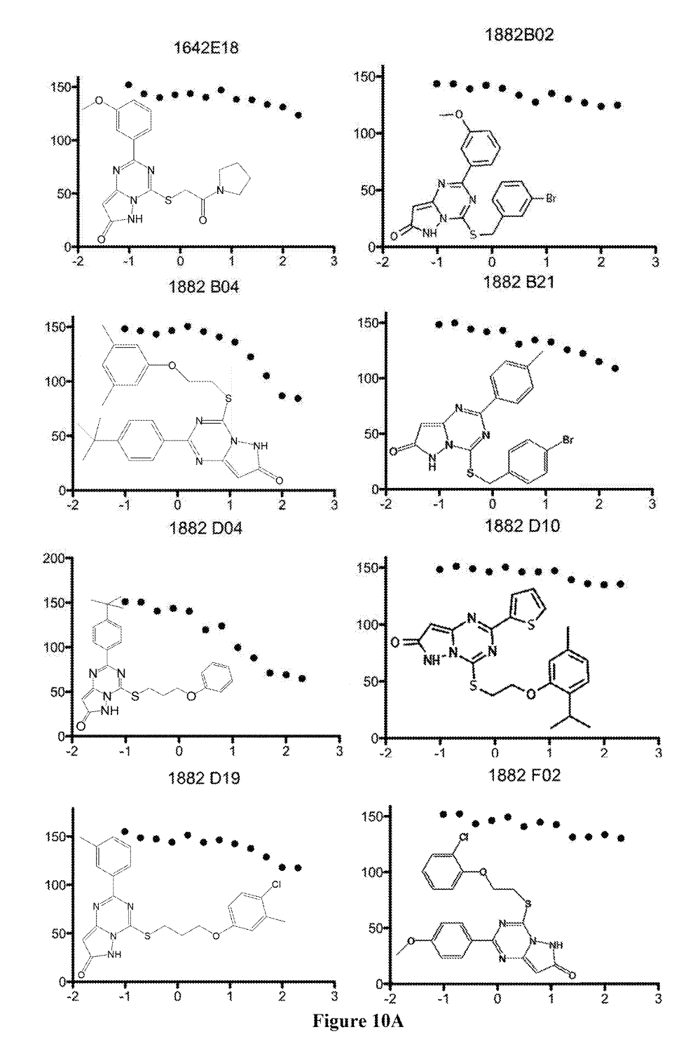

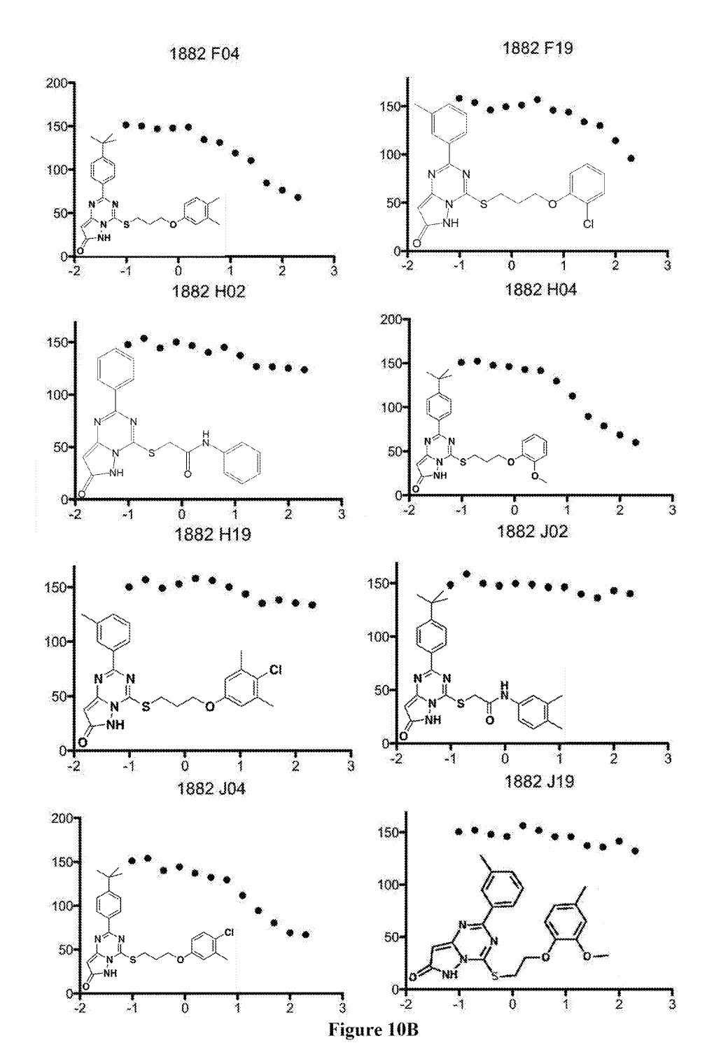

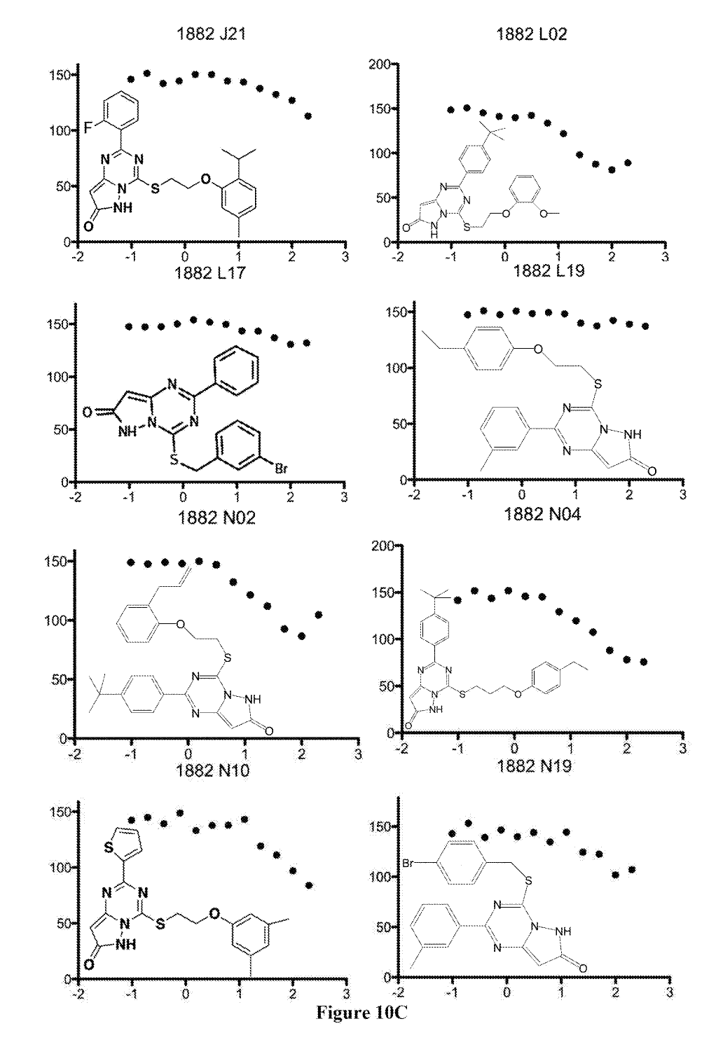

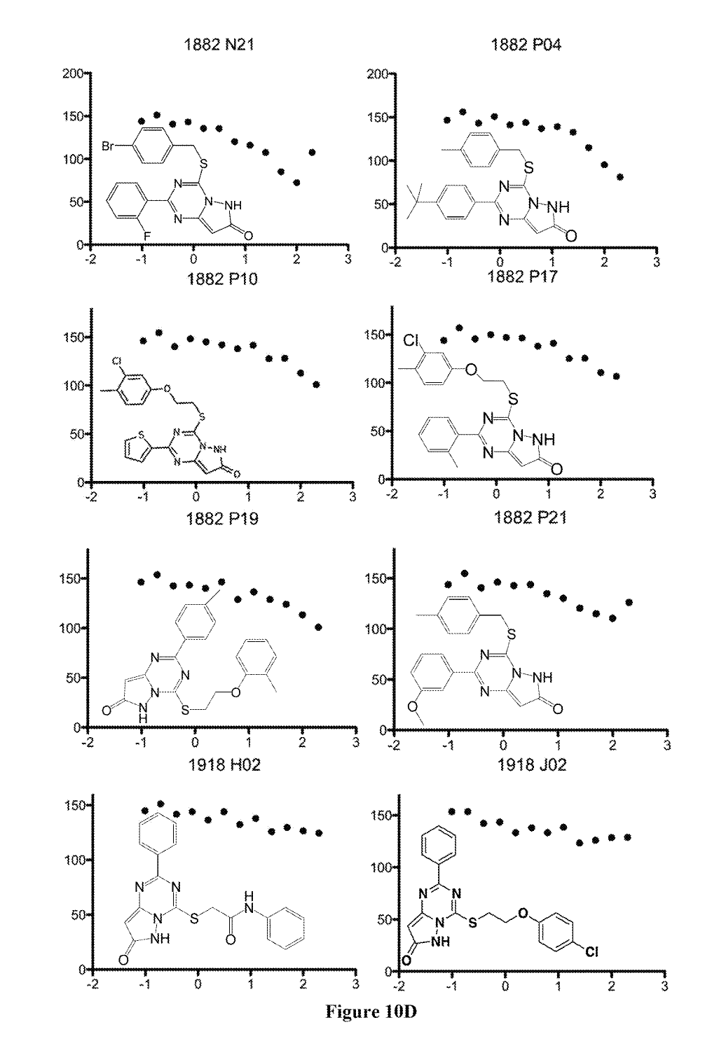

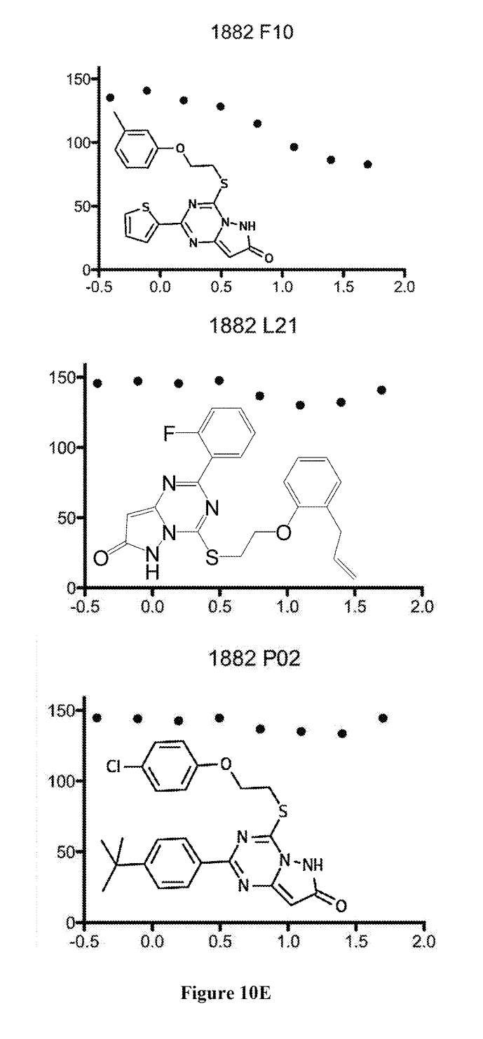

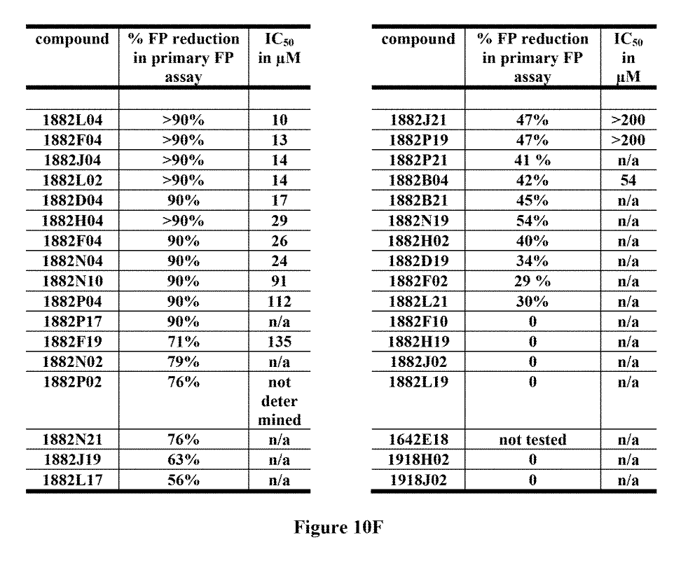

FIGS. 10A-E show values of reduction of fluorescence polarization for exemplified compounds. FIG. 10F shows IC₅₀ values of the exemplified compounds. In the primary assay, FP was read after 30 min at 4° C., compound concentration was ca. 100 μM, and 90% reduction in FP was threshold for scoring hits (Gampe et al., J. Am. Chem. Soc. 2013, 135 (10), 3776-3779; U.S. Provisional Patent Application, U.S. Ser. No. 61/621,229, filed on Apr. 6, 2012; International Application No. PCT/US2013/030800 filed on Mar. 13, 2013).

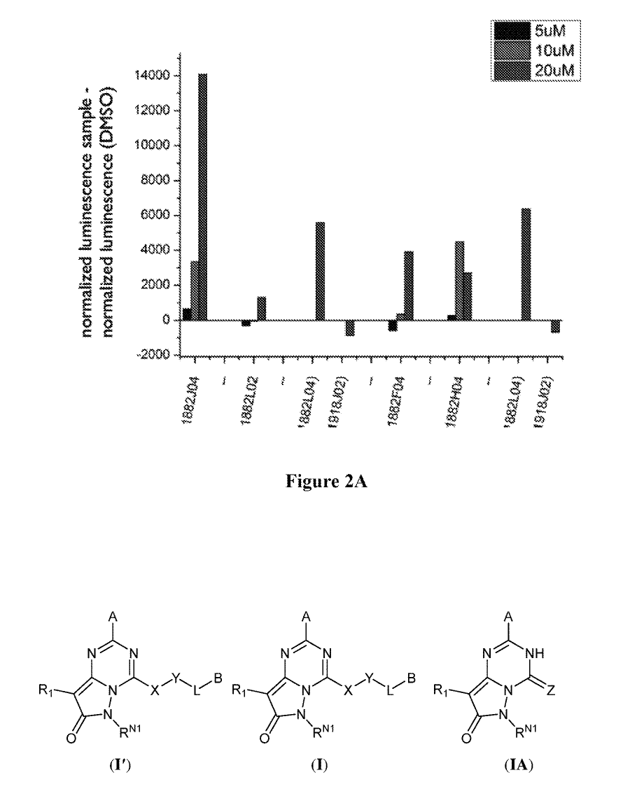

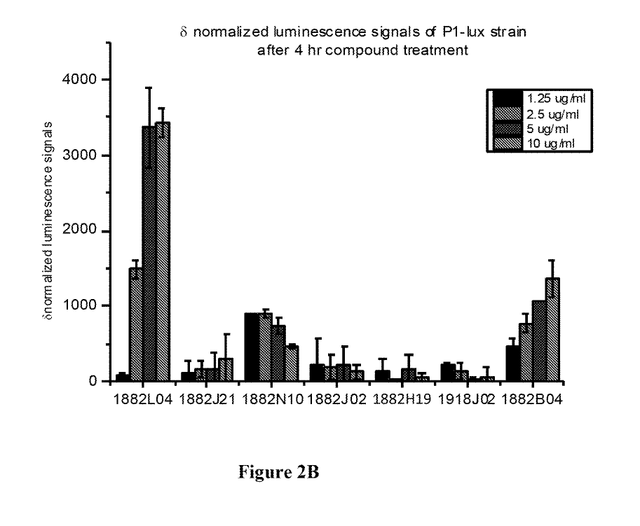

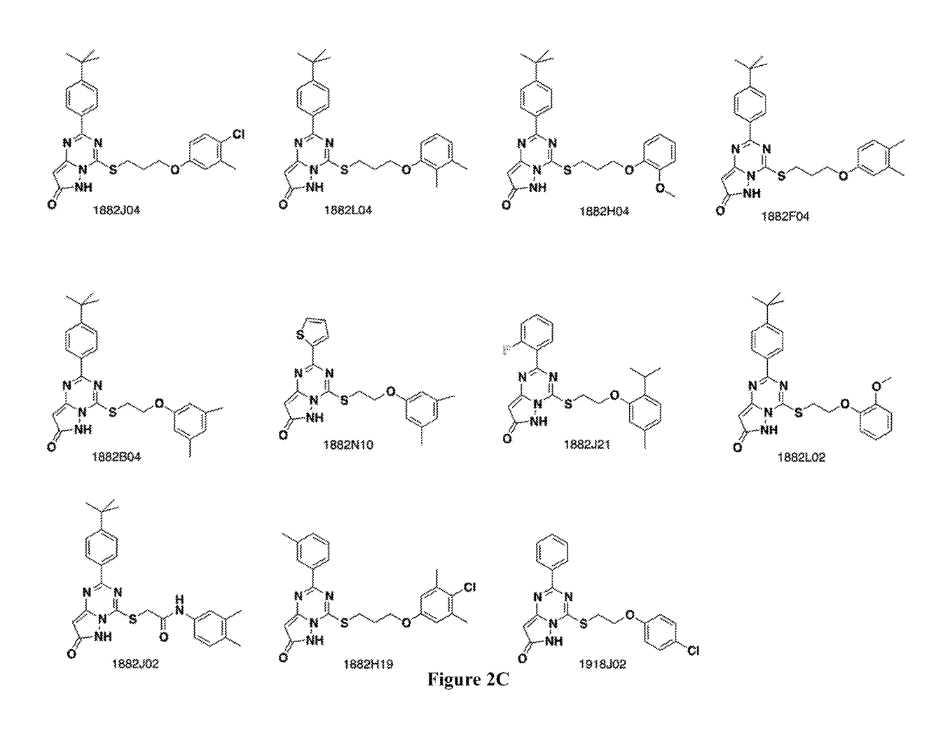

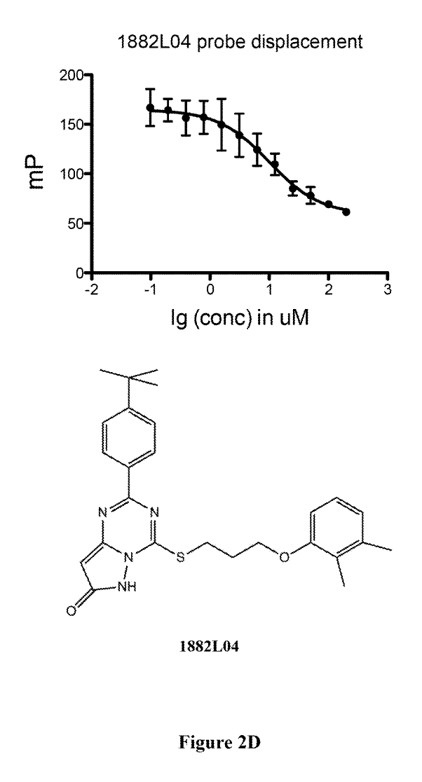

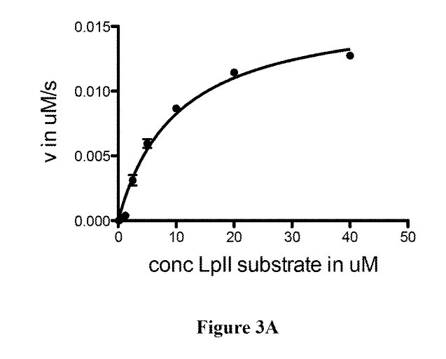

FIG. 2D shows compound 1882L04 displacing probe CMG121 from S. aureus SgtB. The values were determined in two independent experiments. IC₅₀=10 μM; Hill slope: −0.92.

Table 1 shows the IC₅₀ values of exemplified compounds from fluorescence polarization (FP) assay.

TABLE 1
IC₅₀ value of exemplified compounds from FP assay

| | | IC₅₀ |
|---|---|---|
| | | from FP |
| | | Assay |
| Entry | Compound Structure | (μM) |
| 1 | 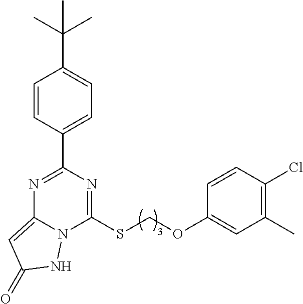 | 14 |
| 2 | 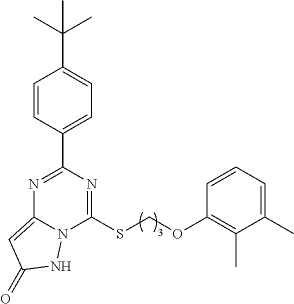 | 12 and 7 |
| 3 | 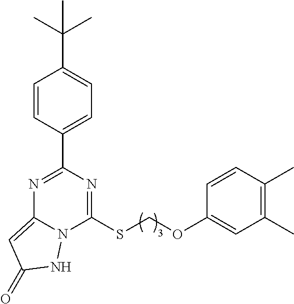 | 13 and 26 |
| 4 | 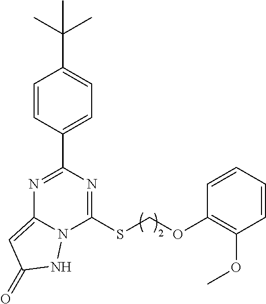 | 14 |
| 5 | 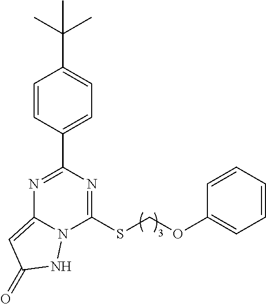 | 17 |
| 6 | 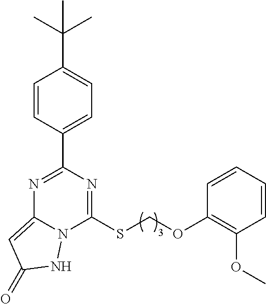 | 29 |
| 7 | 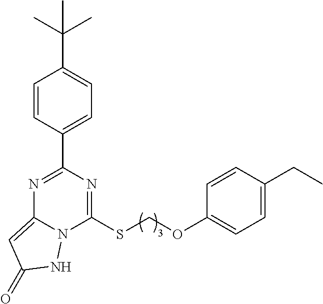 | 24 |
| 8 | 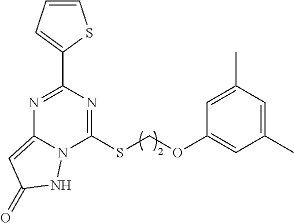 | 91 |
| 9 | 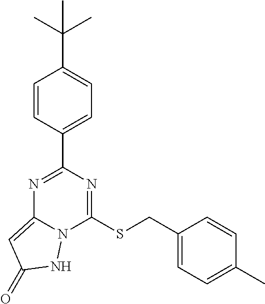 [SMILES: Cc1ccc(CSc2nc(-c3ccc(C(C)(C)C)cc3)nc3cc(=O)[nH]n23)cc1] | 112 |
| 10 | 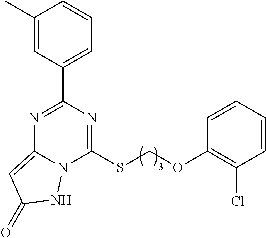 | 135 |
| 11 | 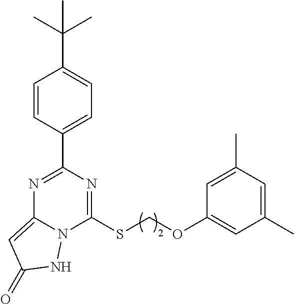 | 54 |
| 12 | 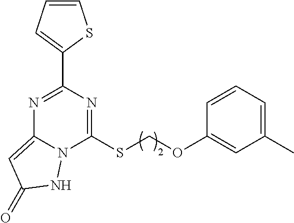 | 9.7 |
| 13 | 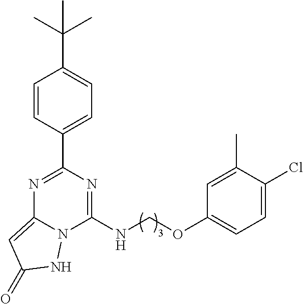 | 21 |
| 14 | 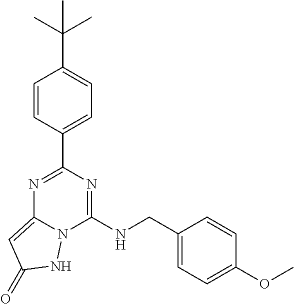 [SMILES: COc1ccc(CNc2nc(-c3ccc(C(C)(C)C)cc3)nc3cc(=O)[nH]n23)cc1] | >200 |
| 15 | 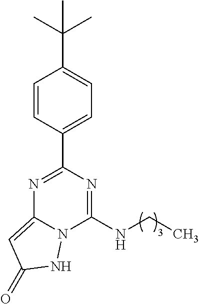 | >200 |
| 16 | 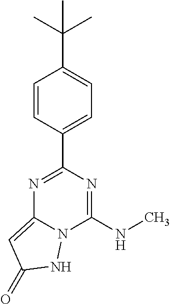 [SMILES: CNc1nc(-c2ccc(C(C)(C)C)cc2)nc2cc(=O)[nH]n12] | >200 |
| 17 | 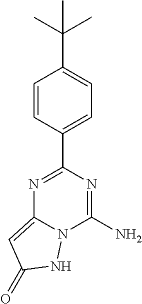 [SMILES: CC(C)(C)c1ccc(-c2nc(N)n3[nH]c(=O)cc3n2)cc1] | >200 |
| 18 | 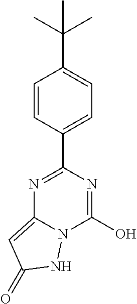 [SMILES: CC(C)(C)c1ccc(-c2nc(O)n3[nH]c(=O)cc3n2)cc1] | 34 |

### II. In Vitro Inhibition of Polymerization of Lipid II by PGTs

The PGT-inhibitor assay was carried out as described previously (Chen et al., Proc. Natl. Acad. Sci. USA 2003, 100, 5658-5663; Wang et al., J. Am. Chem. Soc. 2011, 133, 8528-8530).

(i) In Vitro Inhibition of S. aureus SgtB:

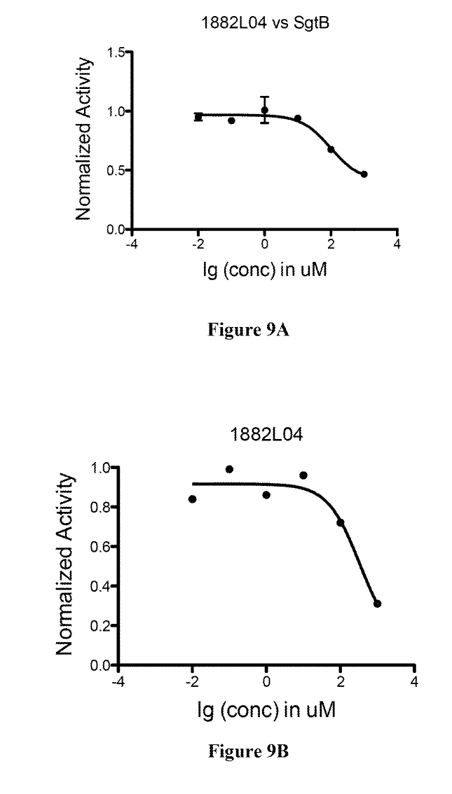

Solutions of S. aureus SgtB (50 nM) in 12.5 mM HEPES (pH=7.5), 2 mM MnCl₂, and 250 μM tween-80 (8 μL) were incubated with DMSO solutions containing compound 1882L04 in different concentrations (1 μL) for 20 min. Then ¹⁴C-labelled lipid II (1 μL, 40 μM, ¹⁴C/¹²C 1/3) was added and the polymerization reaction was allowed to proceed for 25 min at room temperature. The reaction was quenched with 10 μL of a solution of moenomycin (1 μM) in 10% triton-X reduced and the remaining lipid II was separated from peptidoglycan (PG) using paper strip chromatography (isobutyric acid/1M NH₄OH 5/3). Using a scintillation counter the ratio of radioactivity in PG to total radioactivity was determined and plotted vs. inhibitor concentration. IC₅₀ values were determined using the curve fitting program GraphPad Prism 5.0 (GraphPad Software, Inc.; La Jolla, Calif., USA). For compound 1882L04, an IC₅₀ of 97 μM was determined in two independent experiments (FIG. 9A).

(ii) In Vitro Inhibition of E. faecalis PBP2a

Solutions of the E. faecalis PBP2a (50 nM) in 50 mM HEPES (pH=7.5), 10 mM CaCl₂, and 1000 U/min PenG (8 μL) were incubated with DMSO solutions containing the inhibitor of interest in different concentrations (1 μL) for 20 min. Then ¹⁴C-labelled lipid II (1 μL, 40 μM, ¹⁴C/¹²C 1/3) was added and the polymerization reaction was allowed to proceed for 25 min at room temperature. The reaction was quenched and processed as described above. For compound 1882L04, an IC₅₀ of 337 μM was determined (FIG. 9B).

### I. Michaelis-Menten Enzyme Kinetics

(i) Determination of K_{M} and vₘₐₓ for the Uninhibited Polymerization of Lipid II by S. aureus SgtB

Solutions of S. aureus SgtB (50 nM) in 12.5 mM HEPES (pH=7.5), 2 mM MnCl₂, 250 μM tween-80, and 10% DMSO (9 μL total) were incubated with DMSO solutions of radioactively labeled lipid II (1 μL of 10×, ¹⁴C/¹²C=1/3 for c>25 μM; ¹⁴C/¹²C=3/1 for c<25 μM) for 20 min at room temperature.

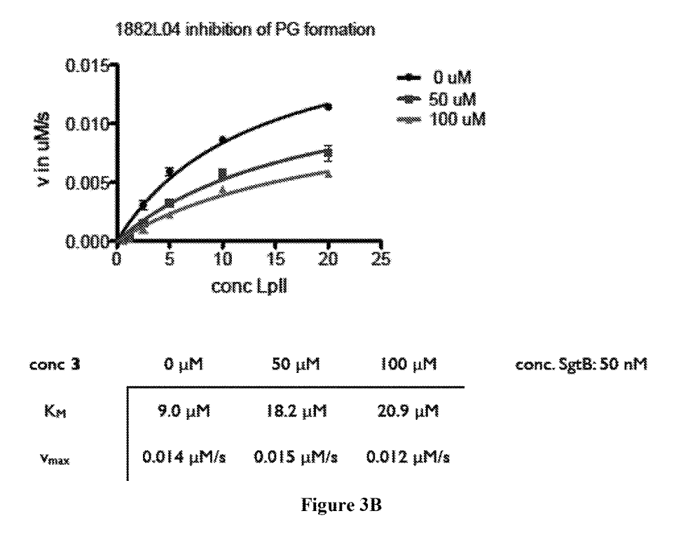

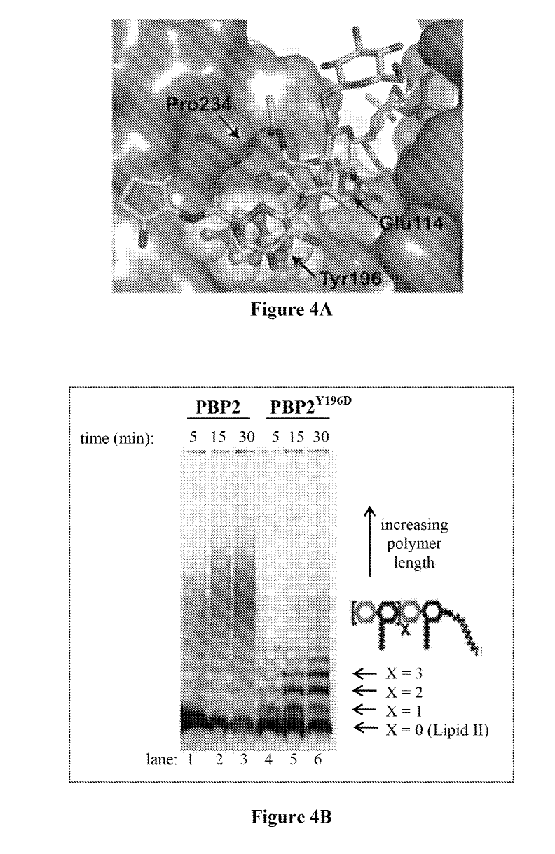

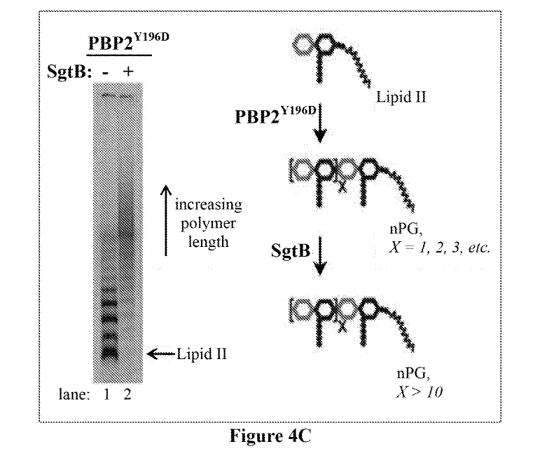

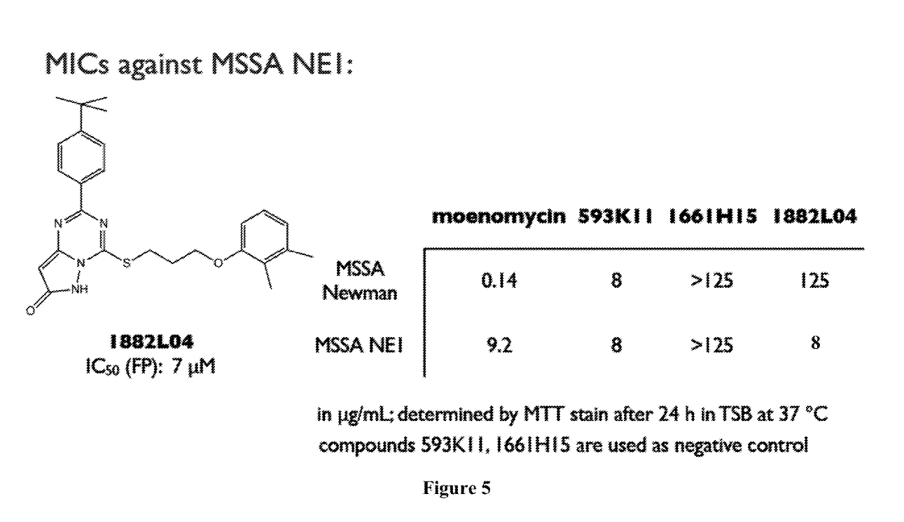

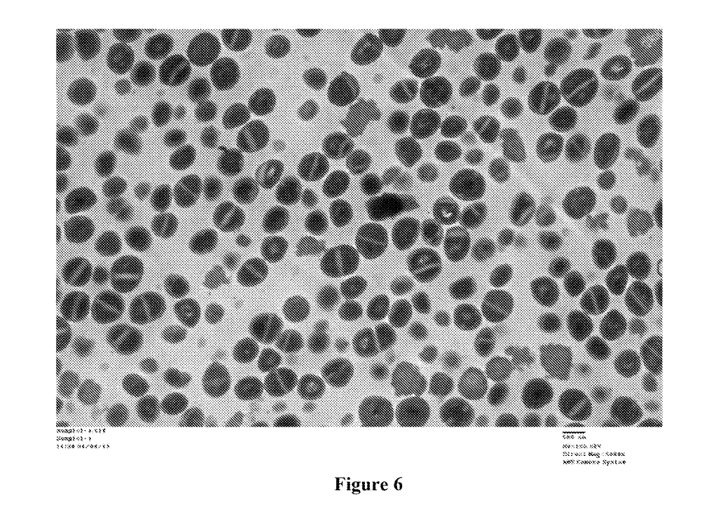

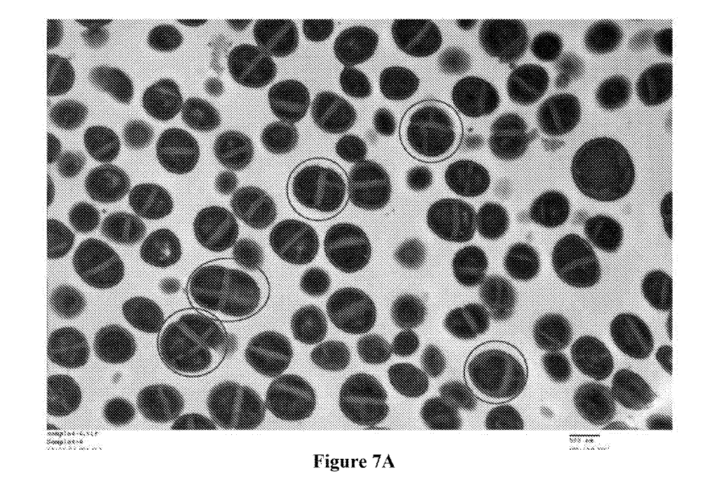

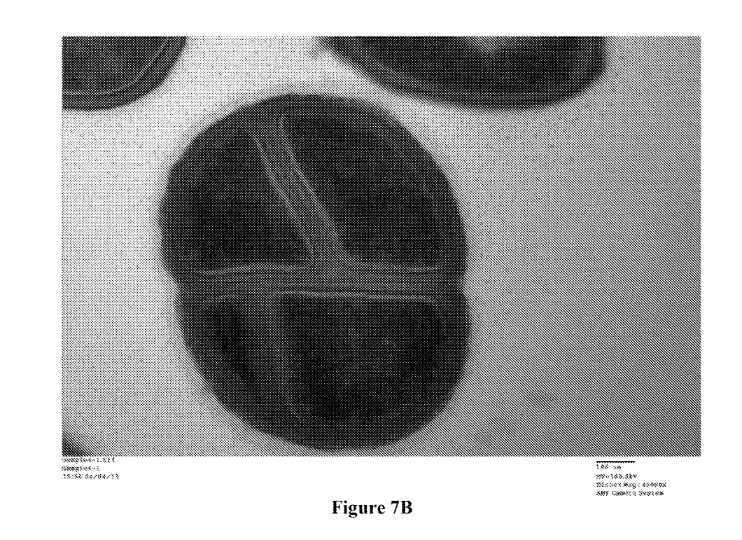

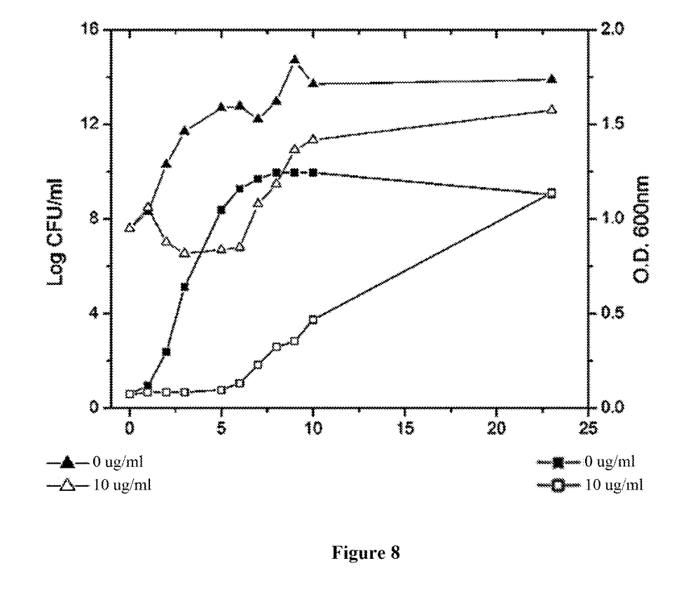

The reaction was quenched with 10 μL of a solution of moenomycin (1 μM) in 10% triton-X reduced and the remaining lipid II was separated from peptidoglycan (PG) using paper strip chromatography (isobutyric acid/1M NH₄OH 5/3). Using a scintillation counter the ratio of radioactivity in PG to total radioactivity was determined. This ratio was multiplied with the concentration of lipid II used to obtain the amount of lipid II in PG in μM. This value was devided by 1200 s to obtain the reaction rate v in μM/s, which was plotted vs. concentration of lipid II. K_{M} and vₘₐₓ were determined using the curve fitting program GraphPad Prism 5.0 (GraphPad Software, Inc.; La Jolla, Calif., USA): K_{M}=10.1 μM; vₘₐₓ=0.017 μM/s (FIG. 3A).

(ii) Determination of K_{M} and vₘₐₓ for Polymerization of Lipid II by S. aureus SgtB in the Presence of 1882L04

Solutions of S. aureus SgtB (50 nM) in 12.5 mM HEPES (pH=7.5), 2 mM MnCl₂, and 250 μM tween-80 (8 μL total) were incubated with a DMSO solution of 1882L04 (10×) for 20 min at room temperature. The polymerization reaction was initiated by addition of radioactively labeled lipid II (1 μL, 100 μM, ¹⁴C/¹²C=1/3; for a final concentration of 10 μM=K_{M}). After 20 min at room temperature, the reaction was quenched with 10 μL of a solution of moenomycin (1 μM) in 10% triton-X reduced and the remaining lipid II was separated from peptidoglycan (PG) using paper strip chromatography (isobutyric acid/1M NH₄OH 5/3). Using a scintillation counter the ratio of radioactivity in PG to total radioactivity was determined. This ratio was multiplied with the concentration of lipid II used to obtain the amount of lipid II in PG in μM. This value was devided by 1200 s to obtain the reaction rate v in μM/s, which was plotted vs. concentration of lipid II. K_{M} and vₘₐₓ were determined using the curve fitting program GraphPad Prism 5.0 (GraphPad Software, Inc.; La Jolla, Calif., USA) (FIG. 3B)

c(1882L04)=0 μM; K_{M}=9.0 μM; vₘₐₓ=0.014 μM/s

c(1882L04)=50 μM; K_{M}=18.2 μM; vₘₐₓ=0.015 μM/s

c(1882L04)=100 μM; K_{M}=20.9 μM; vₘₐₓ=0.012 μM/s

The observed increase of K_{M}, while vₘₐₓ remains the same, is consistent with an interpretation that compound 1882L04 competes with lipid II for binding to the enzyme.

### IV. MIC Test

MIC Test (Methicillin Susceptible Staphylococcus aureus (MSSA Newman) and MSSA NE1)

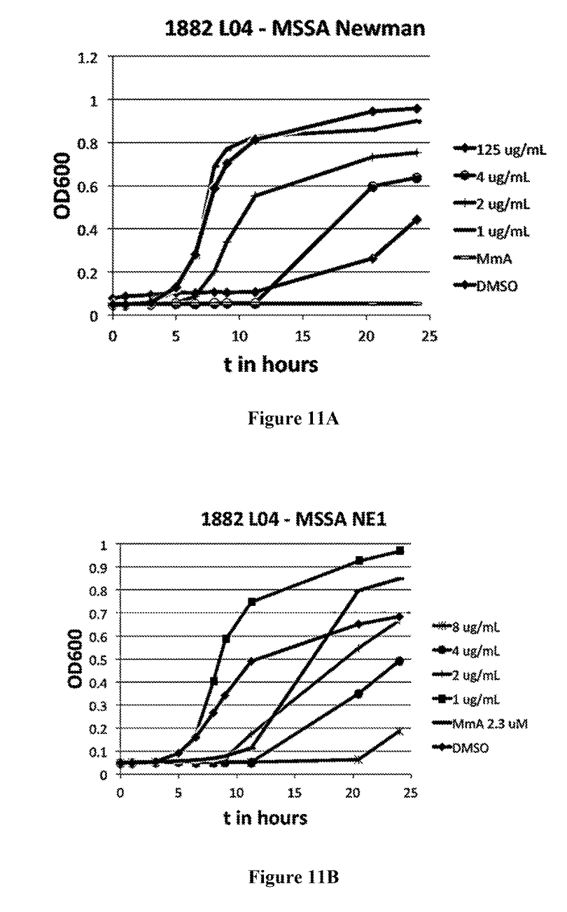

Overnight cultures of MSSA Newman and MSSA NE1 were grown up in TSB medium at 37° C. The cultures obtained were normalized with TSB to O₆₀₀=0.6 and diluted 100-fold. A sterile, clear 96 well plate was charged with 1.5 μL of a dilution series compound 1882L04 in DMSO (100×, in duplicate), and the diluted bacterial culture was added (150 μL). Moenomycin at a final concentration of 2.3 μM was used as positive control. The plate was shaken at 37° C., and the OD₆₀₀ was measured every 1-2 h to obtain the growth curves shown in FIG. 11.

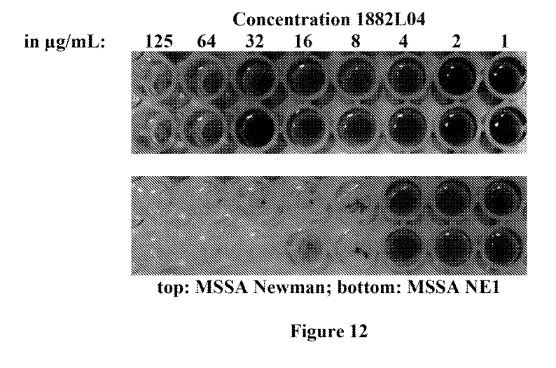

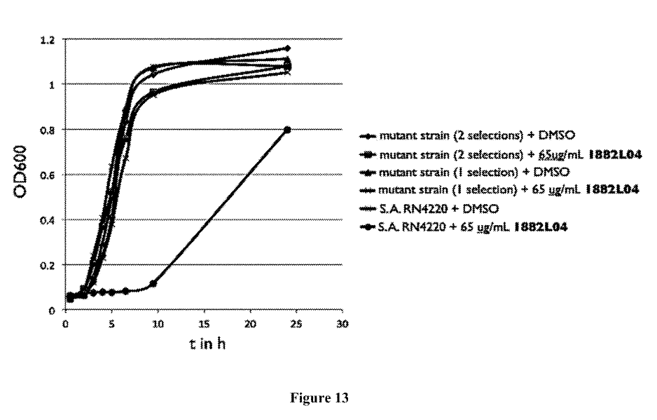

After 22 h, 50 μL of (3-(4,5-dimethylthiazol-2-yl)-2,5-diphenyltetrazolium bromide (MT, 5 mg/mL) were added to stain alive cells. The clear well with the lowest concentration of compound indicates the MIC. For MSSA Newman: 125 μg/mL; for MSSA NE1: 8 μg/mL (FIG. 12).

MIC Test (Methicillin Susceptible Staphylococcus aureus (MSSA Newman))

Overnight cultures of MSSA Newman were grown up in TSB medium at 37° C. The cultures obtained were normalized with TSB to OD₆₀₀=0.6 and diluted 100-fold. 1.5 μL of a dilution series of Example Compounds in DMSO (100×, in duplicate), and the diluted bacterial culture (150 μL) was added to wells of a sterile, clear 96 well plate and incubated at 37° C. After 7 h or 24 h, 50 μL of (3-(4,5-dimethylthiazol-2-yl)-2,5-diphenyltetrazolium bromide (MT, 5 mg/mL) were added to stain alive cells. The clear well with the lowest concentration of compound indicates the MIC. Results are detailed in Table 2 (MSSA Newman 7 h and MSSA Newman 24 h).

MIC Test (S. aureus ATCC 29213, S. aureus ATCC 43300 and S. aureus Newman)

Susceptibility testing of S. aureus ATCC 29213, ATCC 43300 and Newman was performed according to Clinical Laboratory Standard Institute (CLSI) guidelines M7-A9. Test articles were stored at room temperature prior to use, then dissolved in filtered sterile DMSO. Stock DMSO solutions were diluted further in cation-adjusted Mueller Hinton broth (CAMHB) to provide appropriate test concentrations. Cultures of S. aureus (ATCC 29213, ATCC 43300 or Newman) were added in accordance with CLSI guidelines and plates incubated at 37° C. in air for 24 h. Endpoints were determined visually (and by spectrophotometer at 600 nm) and the minimum inhibitory concentration (MIC) defined as the lowest concentration of test articles resulting in complete inhibition of visible bacterial growth. Results are detailed in Table 2 (S. aureus ATCC 29213 and ATCC 43300) and Table 3 (S. aureus ATCC 29213, ATCC 43300 and Newman).

MIC Test (H. influenzae ATCC 49247)

Susceptibility testing of H. influenzae ATCC 49247 was performed according to Clinical Laboratory Standard Institute (CLSI) guidelines M7-A9. Test articles were stored at room temperature prior to use, then dissolved in filtered sterile DMSO. Stock DMSO solutions were diluted further in Haemophilus Test Medium (HTM) to provide appropriate test concentrations. Cultures of H. influenzae ATCC 49247 were added in accordance with CLSI guidelines and plates incubated at 37° C. in air for 24 h. Endpoints were determined visually (and by spectrophotometer at 600 nm) and the minimum inhibitory concentration (MIC) defined as the lowest concentration of test articles resulting in complete inhibition of visible bacterial growth. Results are detailed in Table 4.

Table 2 shows the MIC values of exemplified compounds (S. aureus ATCC 29213 and ATCC 43300; MSSA Newman at 7 hours and 24 hours).

TABLE 2
MIC values of exemplified compounds (S. aureus ATCC 29213 and ATCC 43300;
MSSA Newman at 7 hours and 24 hours)

| | | | | | S. aureus |
|---|---|---|---|---|---|
| | | | | S. aureus | ATCC |
| | | MSSA | MSSA | ATCC | 43300 |
| | | Newman | Newman | 29213 MIC | MIC at |
| Example | | MIC at 7 h | MIC at 24 h | at 24 h | 24 h |
| No. | Compound Structure | (μg/ml) | (μg/ml) | (μg/ml) | (μg/ml) |
| 1 | 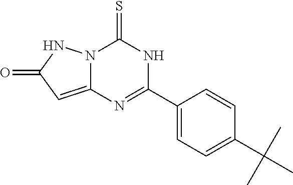 [SMILES: CC(C)(C)c1ccc(-c2nc3cc(=O)[nH]n3c(=S)[nH]2)cc1] | 12.5 | 12.5 | 16 | 16 |
| 2 | 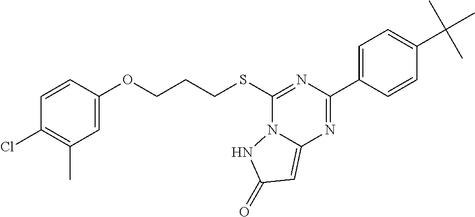 [SMILES: Cc1cc(OCCCSc2nc(-c3ccc(C(C)(C)C)cc3)nc3cc(=O)[nH]n23)ccc1Cl] | >100 | — | >32 | >32 |
| 14 | 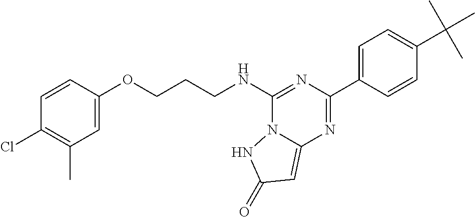 [SMILES: Cc1cc(OCCCNc2nc(-c3ccc(C(C)(C)C)cc3)nc3cc(=O)[nH]n23)ccc1Cl] | 3.3 | 3.3 | 4 | 4 |
| 15 | 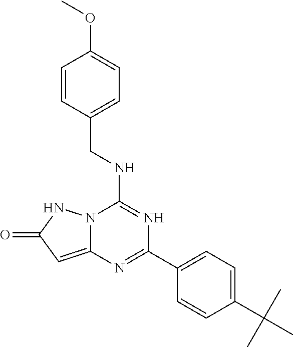 [SMILES: COc1ccc(CNc2nc(-c3ccc(C(C)(C)C)cc3)nc3cc(=O)[nH]n23)cc1] | 3.1 | 12.5 | 8 | 8 |
| 16 | 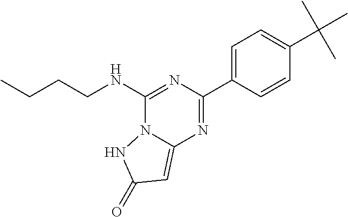 [SMILES: CCCCNc1nc(-c2ccc(C(C)(C)C)cc2)nc2cc(=O)[nH]n12] | 6.2 | 12.5 | — | — |
| 17 | 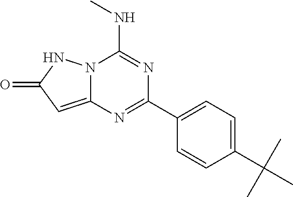 [SMILES: CNc1nc(-c2ccc(C(C)(C)C)cc2)nc2cc(=O)[nH]n12] | 50 | — | — | — |
| 18 | 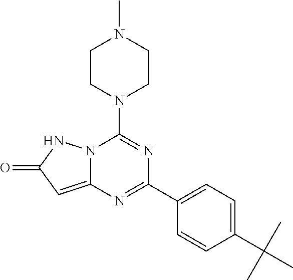 [SMILES: CN1CCN(c2nc(-c3ccc(C(C)(C)C)cc3)nc3cc(=O)[nH]n23)CC1] | — | — | 64 | 64 |
| 19 | 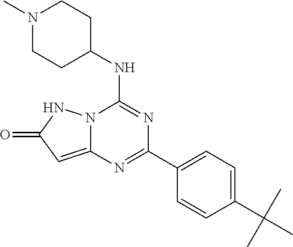 [SMILES: CN1CCC(Nc2nc(-c3ccc(C(C)(C)C)cc3)nc3cc(=O)[nH]n23)CC1] | — | — | 64 | 64 |
| 20 | 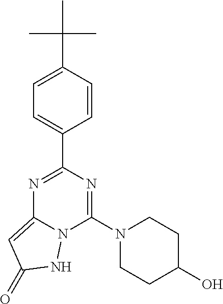 [SMILES: CC(C)(C)c1ccc(-c2nc(N3CCC(O)CC3)n3[nH]c(=O)cc3n2)cc1] | — | — | 64 | 128 |
| 21 | 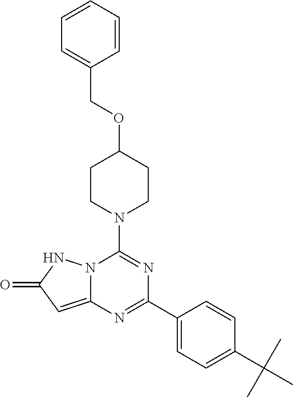 [SMILES: CC(C)(C)c1ccc(-c2nc(N3CCC(OCc4ccccc4)CC3)n3[nH]c(=O)cc3n2)cc1] | — | — | 32 | >128 |
| 22 | 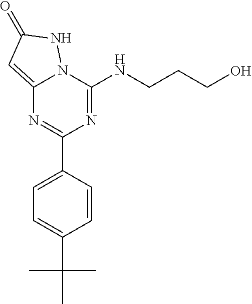 [SMILES: CC(C)(C)c1ccc(-c2nc(NCCCO)n3[nH]c(=O)cc3n2)cc1] | — | — | 8 | 32 |
| 23 | 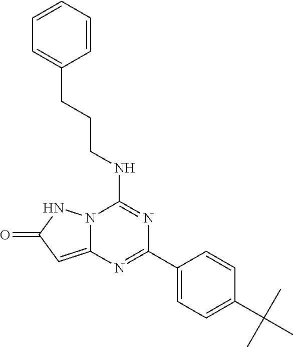 [SMILES: CC(C)(C)c1ccc(-c2nc(NCCCc3ccccc3)n3[nH]c(=O)cc3n2)cc1] | — | — | 4 | 4 |
| 24 | 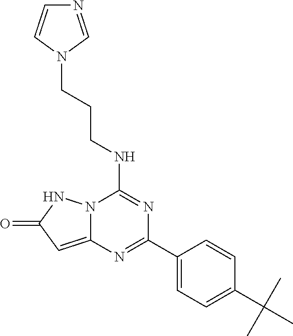 [SMILES: CC(C)(C)c1ccc(-c2nc(NCCCn3ccnc3)n3[nH]c(=O)cc3n2)cc1] | — | — | 64 | 64 |
| 25 | 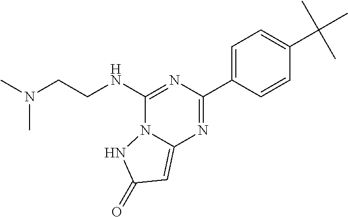 [SMILES: CN(C)CCNc1nc(-c2ccc(C(C)(C)C)cc2)nc2cc(=O)[nH]n12] | — | — | 64 | 32 |
| 26 | 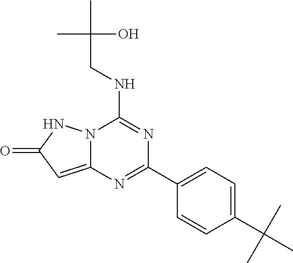 [SMILES: CC(C)(O)CNc1nc(-c2ccc(C(C)(C)C)cc2)nc2cc(=O)[nH]n12] | — | — | 32 | 32 |
| 27 | 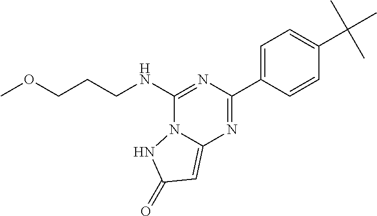 [SMILES: COCCCNc1nc(-c2ccc(C(C)(C)C)cc2)nc2cc(=O)[nH]n12] | 50 | 100 | >32 | >32 |
| 28 | 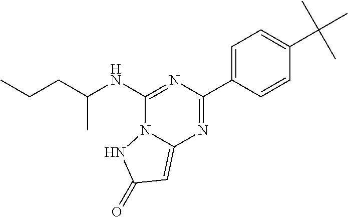 [SMILES: CCCC(C)Nc1nc(-c2ccc(C(C)(C)C)cc2)nc2cc(=O)[nH]n12] | 3.1 | 6.2 | 16 | 16 |
| 29 | 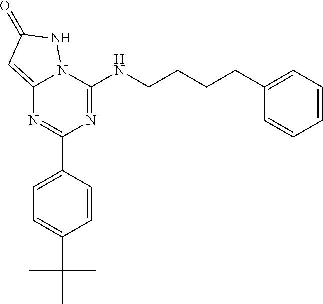 [SMILES: CC(C)(C)c1ccc(-c2nc(NCCCCc3ccccc3)n3[nH]c(=O)cc3n2)cc1] | 1.6 | 3.1 | 8 | 16 |
| 30 | 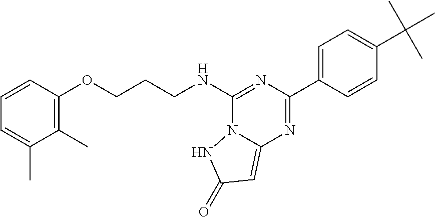 [SMILES: Cc1cccc(OCCCNc2nc(-c3ccc(C(C)(C)C)cc3)nc3cc(=O)[nH]n23)c1C] | 1.6 | 6.2 | >32 | >32 |
| 31 | 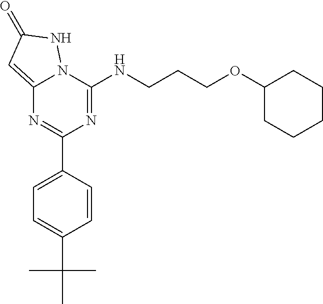 [SMILES: CC(C)(C)c1ccc(-c2nc(NCCCOC3CCCCC3)n3[nH]c(=O)cc3n2)cc1] | — | — | 8 | 16 |
| 32 | 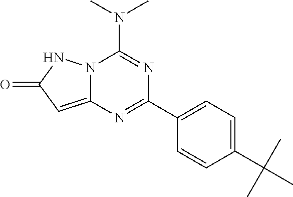 [SMILES: CN(C)c1nc(-c2ccc(C(C)(C)C)cc2)nc2cc(=O)[nH]n12] | — | — | >32 | 32 |
| 33 | 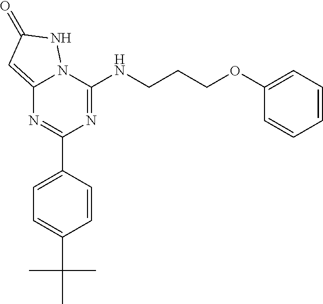 [SMILES: CC(C)(C)c1ccc(-c2nc(NCCCOc3ccccc3)n3[nH]c(=O)cc3n2)cc1] | — | — | 8 | 8 |
| 34 | 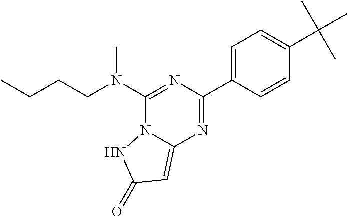 [SMILES: CCCCN(C)c1nc(-c2ccc(C(C)(C)C)cc2)nc2cc(=O)[nH]n12] | — | — | 16 | 16 |
| 35 | 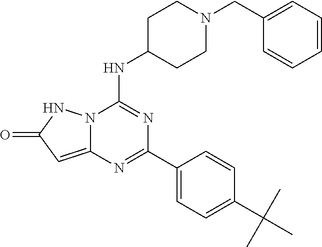 [SMILES: CC(C)(C)c1ccc(-c2nc(NC3CCN(Cc4ccccc4)CC3)n3[nH]c(=O)cc3n2)cc1] | — | — | 8 | 8 |
| 36 | 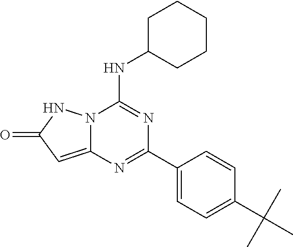 [SMILES: CC(C)(C)c1ccc(-c2nc(NC3CCCCC3)n3[nH]c(=O)cc3n2)cc1] | — | — | 8 | 8 |
| 37 | 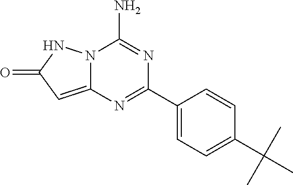 [SMILES: CC(C)(C)c1ccc(-c2nc(N)n3[nH]c(=O)cc3n2)cc1] | 25 | — | — | — |
| 38 | 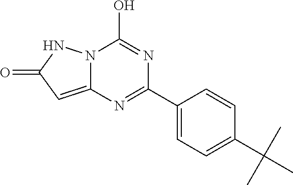 [SMILES: CC(C)(C)c1ccc(-c2nc(O)n3[nH]c(=O)cc3n2)cc1] | 12.5 | 12.5 | — | — |
| 39 | 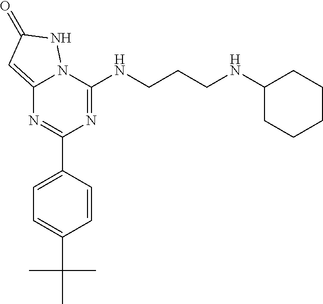 [SMILES: CC(C)(C)c1ccc(-c2nc(NCCCNC3CCCCC3)n3[nH]c(=O)cc3n2)cc1] | — | — | 32 | 32 |
| 40 | 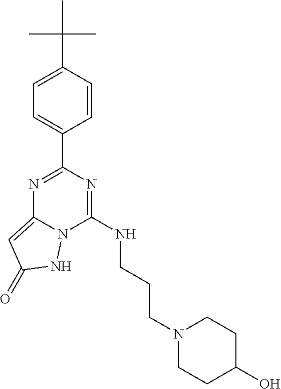 [SMILES: CC(C)(C)c1ccc(-c2nc(NCCCN3CCC(O)CC3)n3[nH]c(=O)cc3n2)cc1] | — | — | 128 | 128 |
| 41 | 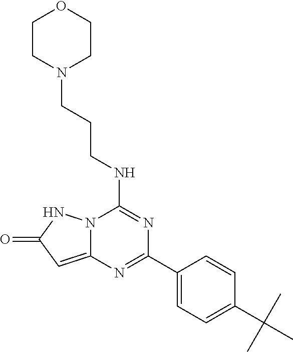 [SMILES: CC(C)(C)c1ccc(-c2nc(NCCCN3CCOCC3)n3[nH]c(=O)cc3n2)cc1] | — | — | 128 | 128 |
| 42 | 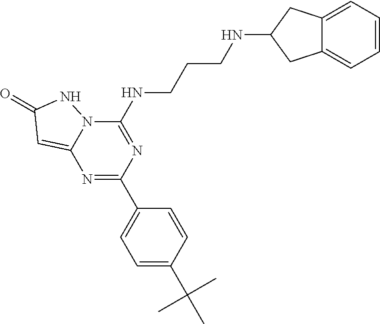 [SMILES: CC(C)(C)c1ccc(-c2nc(NCCCNC3Cc4ccccc4C3)n3[nH]c(=O)cc3n2)cc1] | — | — | 8 | 8 |
| 43 | 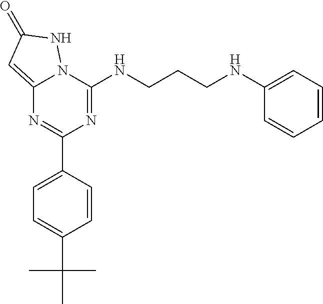 [SMILES: CC(C)(C)c1ccc(-c2nc(NCCCNc3ccccc3)n3[nH]c(=O)cc3n2)cc1] | — | — | 8 | 8 |
| 44 | 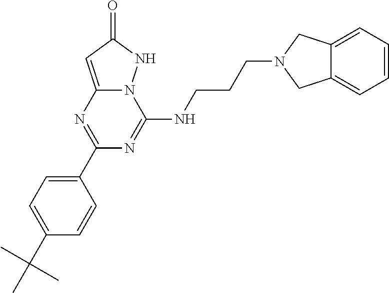 [SMILES: CC(C)(C)c1ccc(-c2nc(NCCCN3Cc4ccccc4C3)n3[nH]c(=O)cc3n2)cc1] | — | — | 64 | >128 |
| 45 | 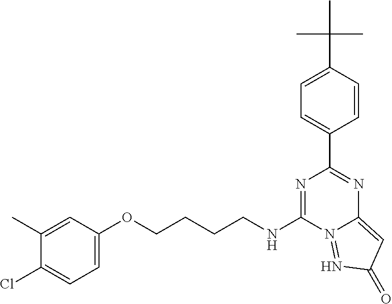 | >100 | >100 | — | — |
| 46 | 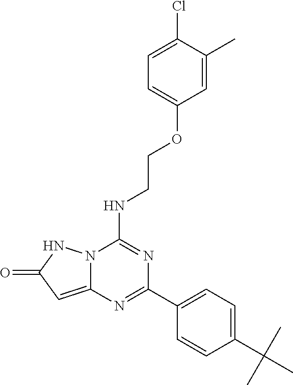 [SMILES: Cc1cc(OCCNc2nc(-c3ccc(C(C)(C)C)cc3)nc3cc(=O)[nH]n23)ccc1Cl] | 12.5 | 12.5 | — | — |
| 47 | 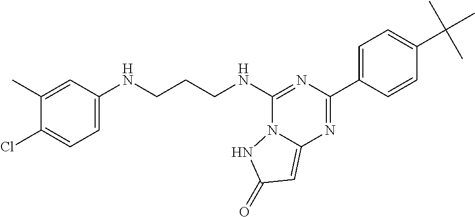 [SMILES: Cc1cc(NCCCNc2nc(-c3ccc(C(C)(C)C)cc3)nc3cc(=O)[nH]n23)ccc1Cl] | 3.1 | 3.1 | 2 | 4 |
| 48 | 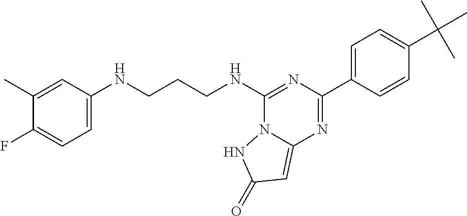 [SMILES: Cc1cc(NCCCNc2nc(-c3ccc(C(C)(C)C)cc3)nc3cc(=O)[nH]n23)ccc1F] | 3.1 | 3.1 | 16 | 8 |
| 49 | 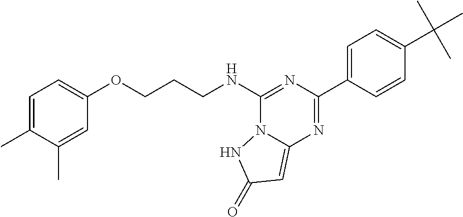 [SMILES: Cc1ccc(OCCCNc2nc(-c3ccc(C(C)(C)C)cc3)nc3cc(=O)[nH]n23)cc1C] | 3.1 | 6.2 | 32 | 16 |
| 50 | 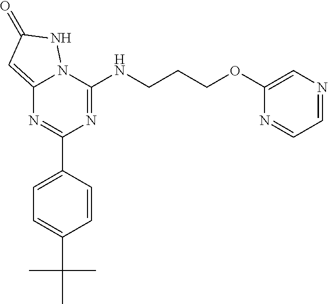 [SMILES: CC(C)(C)c1ccc(-c2nc(NCCCOc3cnccn3)n3[nH]c(=O)cc3n2)cc1] | — | — | >32 | >32 |
| 51 | 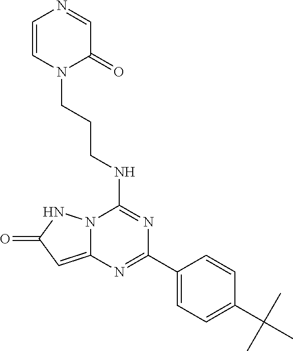 [SMILES: CC(C)(C)c1ccc(-c2nc(NCCCn3ccncc3=O)n3[nH]c(=O)cc3n2)cc1] | — | — | >64 | >64 |
| 52 | 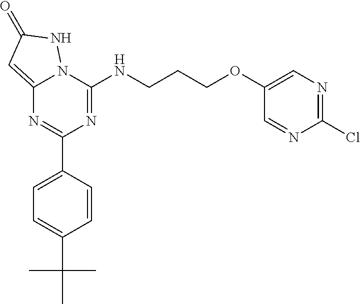 [SMILES: CC(C)(C)c1ccc(-c2nc(NCCCOc3cnc(Cl)nc3)n3[nH]c(=O)cc3n2)cc1] | 12.5 | 25 | >64 | >64 |
| 53 | 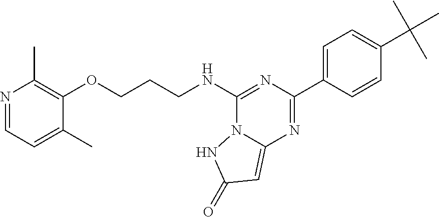 [SMILES: Cc1ccnc(C)c1OCCCNc1nc(-c2ccc(C(C)(C)C)cc2)nc2cc(=O)[nH]n12] | >100 | >100 | >64 | 8 |
| 54 | 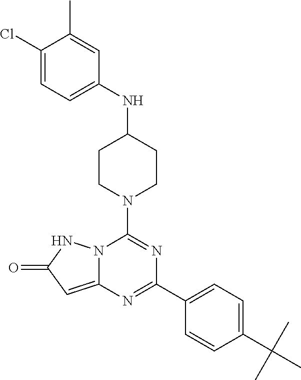 [SMILES: Cc1cc(NC2CCN(c3nc(-c4ccc(C(C)(C)C)cc4)nc4cc(=O)[nH]n34)CC2)ccc1Cl] | — | — | 4 | 4 |
| 55 | 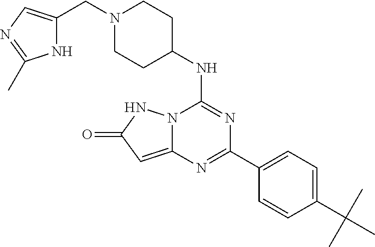 [SMILES: Cc1ncc(CN2CCC(Nc3nc(-c4ccc(C(C)(C)C)cc4)nc4cc(=O)[nH]n34)CC2)[nH]1] | — | — | 32 | 32 |
| 56 | 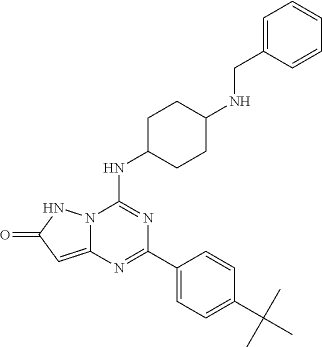 [SMILES: CC(C)(C)c1ccc(-c2nc(NC3CCC(NCc4ccccc4)CC3)n3[nH]c(=O)cc3n2)cc1] | — | — | 16 | 8 |
| 57 | 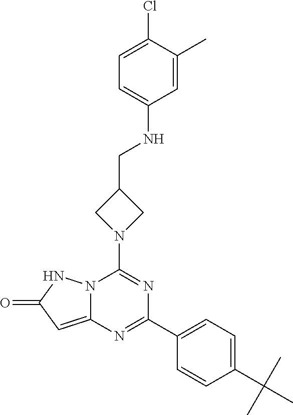 [SMILES: Cc1cc(NCC2CN(c3nc(-c4ccc(C(C)(C)C)cc4)nc4cc(=O)[nH]n34)C2)ccc1Cl] | — | — | 4 | 8 |
| 58 | 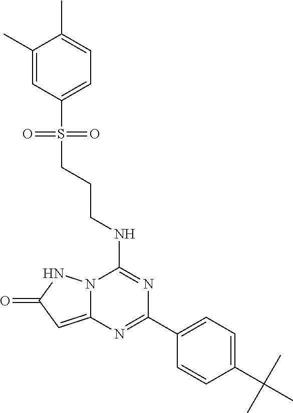 [SMILES: Cc1ccc(S(=O)(=O)CCCNc2nc(-c3ccc(C(C)(C)C)cc3)nc3cc(=O)[nH]n23)cc1C] | — | — | 64 | 64 |
| 59 | 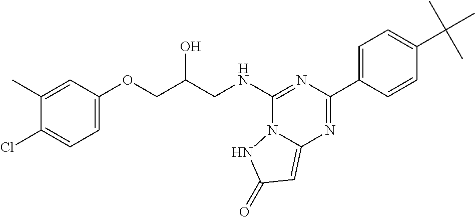 [SMILES: Cc1cc(OCC(O)CNc2nc(-c3ccc(C(C)(C)C)cc3)nc3cc(=O)[nH]n23)ccc1Cl] | — | — | 4 | 4 |
| 60 | 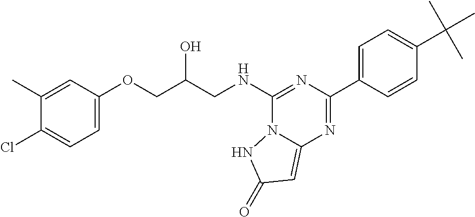 [SMILES: Cc1cc(OCC(O)CNc2nc(-c3ccc(C(C)(C)C)cc3)nc3cc(=O)[nH]n23)ccc1Cl] | — | — | 4 | 8 |
| 61 | 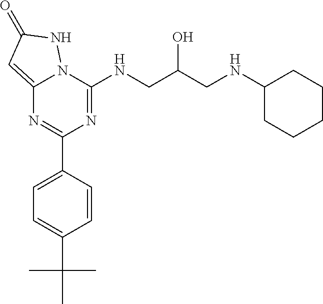 [SMILES: CC(C)(C)c1ccc(-c2nc(NCC(O)CNC3CCCCC3)n3[nH]c(=O)cc3n2)cc1] | — | — | 32 | 32 |
| 62 | 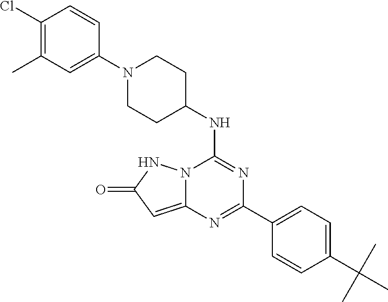 [SMILES: Cc1cc(N2CCC(Nc3nc(-c4ccc(C(C)(C)C)cc4)nc4cc(=O)[nH]n34)CC2)ccc1Cl] | — | — | 4 | 16 |
| 63 | 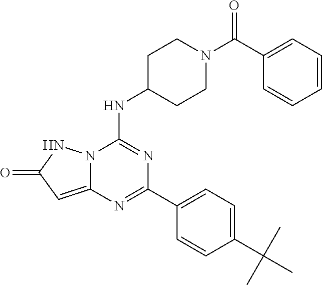 [SMILES: CC(C)(C)c1ccc(-c2nc(NC3CCN(C(=O)c4ccccc4)CC3)n3[nH]c(=O)cc3n2)cc1] | — | — | 32 | 128 |
| 64 | 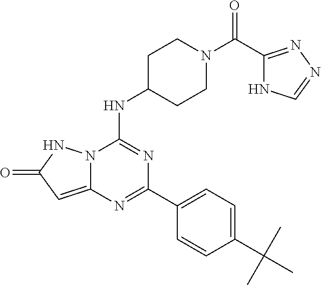 [SMILES: CC(C)(C)c1ccc(-c2nc(NC3CCN(C(=O)c4nnc[nH]4)CC3)n3[nH]c(=O)cc3n2)cc1] | — | — | 128 | >128 |
| 65 | 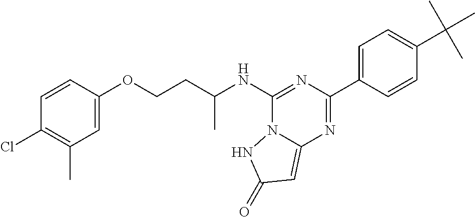 [SMILES: Cc1cc(OCCC(C)Nc2nc(-c3ccc(C(C)(C)C)cc3)nc3cc(=O)[nH]n23)ccc1Cl] | — | — | 4 | 4 |
| 66 | 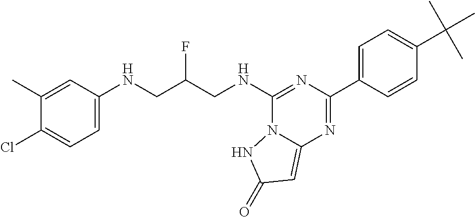 [SMILES: Cc1cc(NCC(F)CNc2nc(-c3ccc(C(C)(C)C)cc3)nc3cc(=O)[nH]n23)ccc1Cl] | — | — | 4 | 2 |
| 67 | 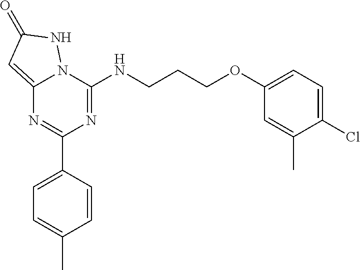 [SMILES: Cc1ccc(-c2nc(NCCCOc3ccc(Cl)c(C)c3)n3[nH]c(=O)cc3n2)cc1] | — | — | >64 | >64 |
| 68 | 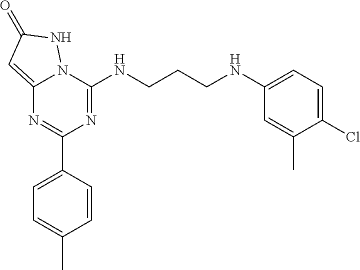 [SMILES: Cc1ccc(-c2nc(NCCCNc3ccc(Cl)c(C)c3)n3[nH]c(=O)cc3n2)cc1] | 1.6 | 3.1 | 16 | 8 |
| 69 | 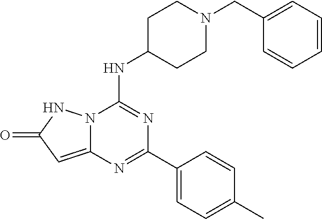 [SMILES: Cc1ccc(-c2nc(NC3CCN(Cc4ccccc4)CC3)n3[nH]c(=O)cc3n2)cc1] | — | — | 32 | >32 |
| 70 | 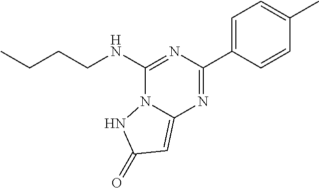 [SMILES: CCCCNc1nc(-c2ccc(C)cc2)nc2cc(=O)[nH]n12] | >100 | >100 | — | — |
| 71 | 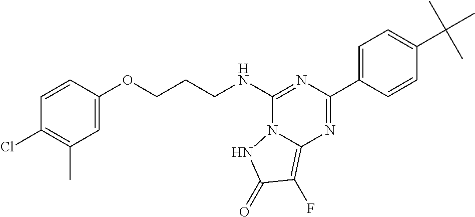 [SMILES: Cc1cc(OCCCNc2nc(-c3ccc(C(C)(C)C)cc3)nc3c(F)c(=O)[nH]n23)ccc1Cl] | — | — | 8 | 8 |
| 72 | 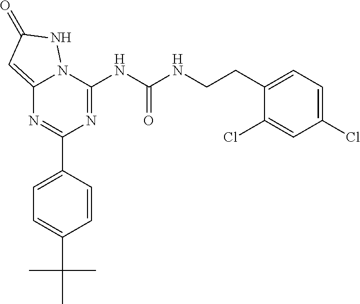 [SMILES: CC(C)(C)c1ccc(-c2nc(NC(=O)NCCc3ccc(Cl)cc3Cl)n3[nH]c(=O)cc3n2)cc1] | 6.2 | >100 | 8 | 16 |

Table 3 shows MIC values of exemplified compounds (S. aureus ATCC 29213, ATCC 43300 and Newman).

TABLE 3
MIC values of Exemplified compounds (S. aureus ATCC 29213, ATCC 43300 and
Newman).

| | | S. | S. | S. |
|---|---|---|---|---|
| | | aureus | aureus | aureus |
| Ex- | | ATCC | ATCC | ATCC |
| am- | | 29213 | 43300 | Newman |
| ple | | MIC | MIC | MIC |
| No. | Compound Structure | (μg/ml) | (μg/ml) | (μg/ml) |
| 73 | 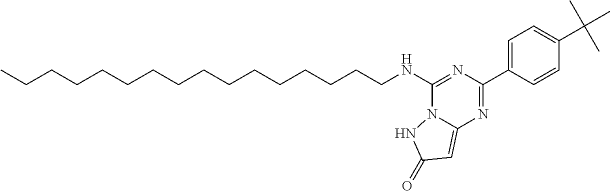 [SMILES: CCCCCCCCCCCCCCCCNc1nc(-c2ccc(C(C)(C)C)cc2)nc2cc(=O)[nH]n12] | >32 | >32 | >32 |
| 74 | 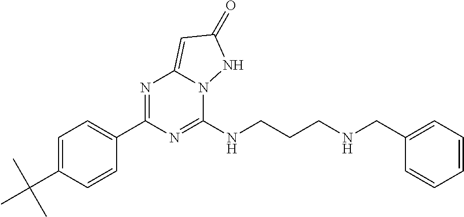 [SMILES: CC(C)(C)c1ccc(-c2nc(NCCCNCc3ccccc3)n3[nH]c(=O)cc3n2)cc1] | 16 | 16 | 16 |
| 75 | 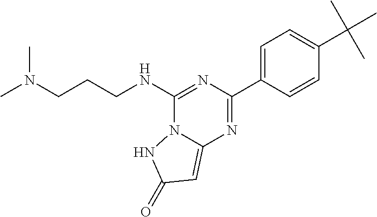 [SMILES: CN(C)CCCNc1nc(-c2ccc(C(C)(C)C)cc2)nc2cc(=O)[nH]n12] | 32 | 32 | 32 |
| 76 | 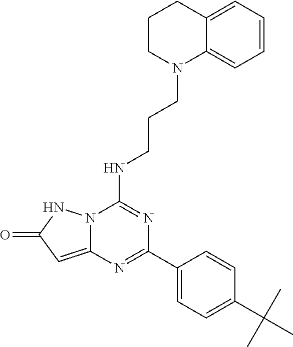 [SMILES: CC(C)(C)c1ccc(-c2nc(NCCCN3CCCc4ccccc43)n3[nH]c(=O)cc3n2)cc1] | 4 | — | 4 |
| 77 | 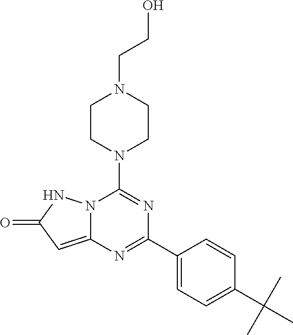 [SMILES: CC(C)(C)c1ccc(-c2nc(N3CCN(CCO)CC3)n3[nH]c(=O)cc3n2)cc1] | 64 | — | 64 |
| 78 | 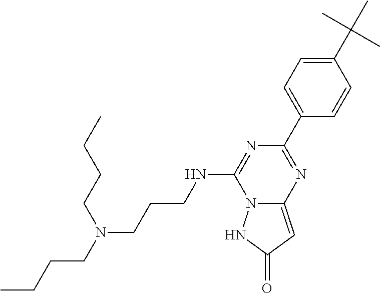 [SMILES: CCCCN(CCCC)CCCNc1nc(-c2ccc(C(C)(C)C)cc2)nc2cc(=O)[nH]n12] | 32 | — | 32 |
| 79 | 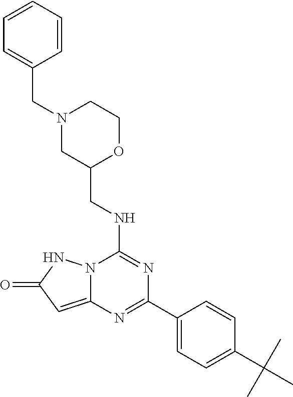 [SMILES: CC(C)(C)c1ccc(-c2nc(NCC3CN(Cc4ccccc4)CCO3)n3[nH]c(=O)cc3n2)cc1] | 32 | — | 16 |
| 80 | 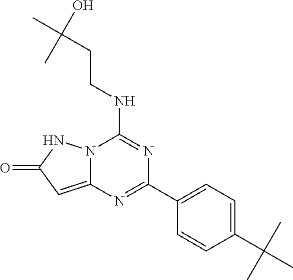 [SMILES: CC(C)(O)CCNc1nc(-c2ccc(C(C)(C)C)cc2)nc2cc(=O)[nH]n12] | 32 | 64 | 32 |
| 81 | 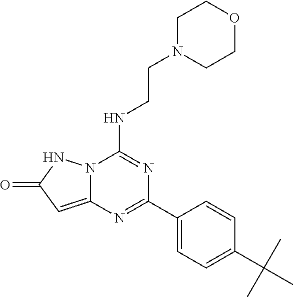 [SMILES: CC(C)(C)c1ccc(-c2nc(NCCN3CCOCC3)n3[nH]c(=O)cc3n2)cc1] | 128 | 128 | 128 |
| 82 | 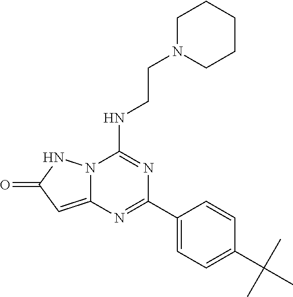 [SMILES: CC(C)(C)c1ccc(-c2nc(NCCN3CCCCC3)n3[nH]c(=O)cc3n2)cc1] | 64 | 64 | 32 |
| 83 | 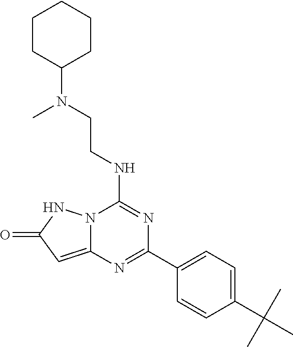 [SMILES: CN(CCNc1nc(-c2ccc(C(C)(C)C)cc2)nc2cc(=O)[nH]n12)C1CCCCC1] | 32 | 32 | 32 |
| 84 | 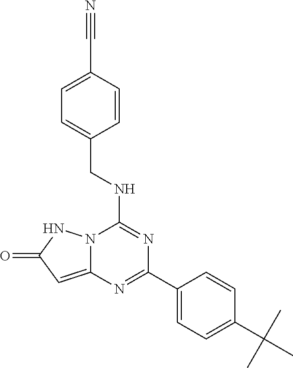 [SMILES: CC(C)(C)c1ccc(-c2nc(NCc3ccc(C#N)cc3)n3[nH]c(=O)cc3n2)cc1] | 16 | — | 8 |
| 85 | 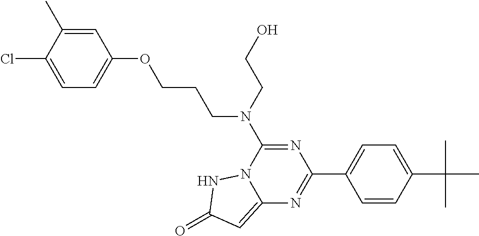 [SMILES: Cc1cc(OCCCN(CCO)c2nc(-c3ccc(C(C)(C)C)cc3)nc3cc(=O)[nH]n23)ccc1Cl] | 32 | 32 | 4 |
| 86 | 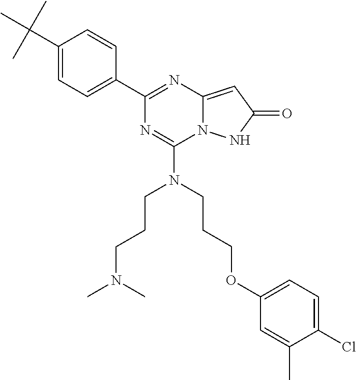 [SMILES: Cc1cc(OCCCN(CCCN(C)C)c2nc(-c3ccc(C(C)(C)C)cc3)nc3cc(=O)[nH]n23)ccc1Cl] | 2 | — | 1 |
| 87 | 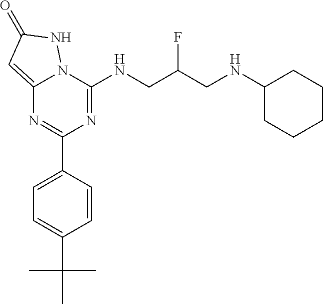 [SMILES: CC(C)(C)c1ccc(-c2nc(NCC(F)CNC3CCCCC3)n3[nH]c(=O)cc3n2)cc1] | 32 | 32 | 32 |
| 88 | 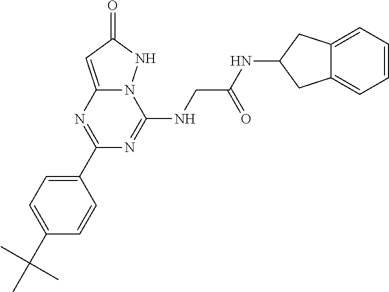 [SMILES: CC(C)(C)c1ccc(-c2nc(NCC(=O)NC3Cc4ccccc4C3)n3[nH]c(=O)cc3n2)cc1] | >128 | — | 128 |
| 89 | 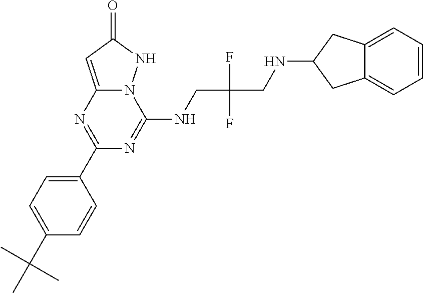 [SMILES: CC(C)(C)c1ccc(-c2nc(NCC(F)(F)CNC3Cc4ccccc4C3)n3[nH]c(=O)cc3n2)cc1] | 8 | — | 8 |
| 90 | 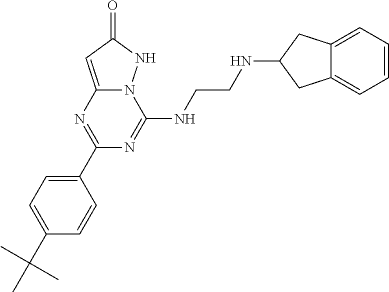 [SMILES: CC(C)(C)c1ccc(-c2nc(NCCNC3Cc4ccccc4C3)n3[nH]c(=O)cc3n2)cc1] | 16 | 16 | 16 |
| 91 | 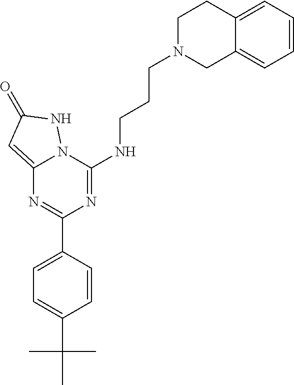 [SMILES: CC(C)(C)c1ccc(-c2nc(NCCCN3CCc4ccccc4C3)n3[nH]c(=O)cc3n2)cc1] | 32 | — | 16 |
| 92 | 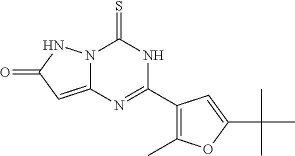 [SMILES: Cc1oc(C(C)(C)C)cc1-c1nc2cc(=O)[nH]n2c(=S)[nH]1] | 32 | 16 | 8 |
| 93 | 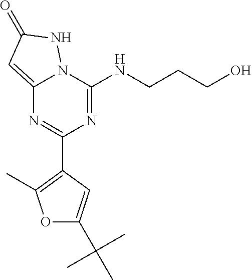 [SMILES: Cc1oc(C(C)(C)C)cc1-c1nc(NCCCO)n2[nH]c(=O)cc2n1] | 32 | 32 | 16 |
| 94 | 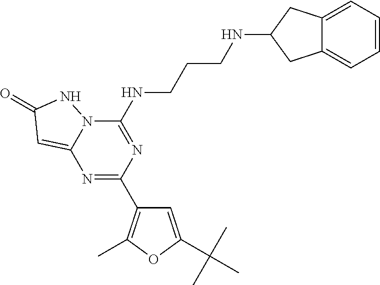 [SMILES: Cc1oc(C(C)(C)C)cc1-c1nc(NCCCNC2Cc3ccccc3C2)n2[nH]c(=O)cc2n1] | 16 | 16 | 16 |
| 95 | 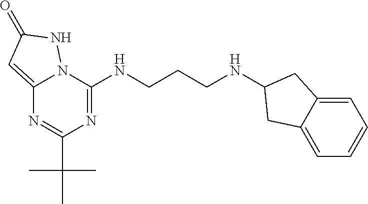 [SMILES: CC(C)(C)c1nc(NCCCNC2Cc3ccccc3C2)n2[nH]c(=O)cc2n1] | 16 | — | 8 |
| 96 | 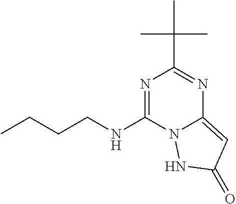 [SMILES: CCCCNc1nc(C(C)(C)C)nc2cc(=O)[nH]n12] | 128 | — | 32 |
| 97 | 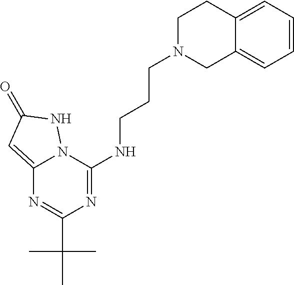 [SMILES: CC(C)(C)c1nc(NCCCN2CCc3ccccc3C2)n2[nH]c(=O)cc2n1] | 128 | — | 64 |
| 98 | 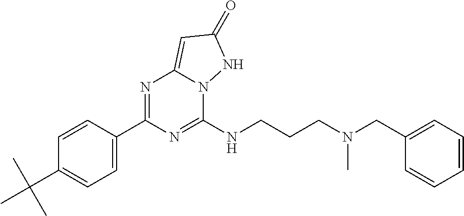 [SMILES: CN(CCCNc1nc(-c2ccc(C(C)(C)C)cc2)nc2cc(=O)[nH]n12)Cc1ccccc1] | 32 | — | 32 |
| 99 | 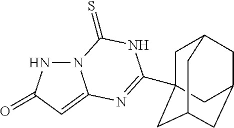 [SMILES: O=c1cc2nc(C34CC5CC(CC(C5)C3)C4)[nH]c(=S)n2[nH]1] | 64 | 64 | 32 |
| 100 | 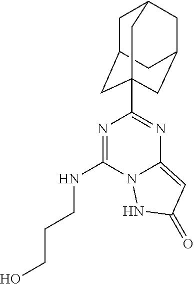 [SMILES: O=c1cc2nc(C34CC5CC(CC(C5)C3)C4)nc(NCCCO)n2[nH]1] | 32 | 32 | 16 |
| 101 | 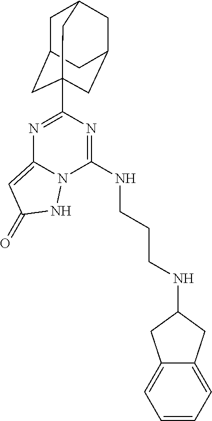 [SMILES: O=c1cc2nc(C34CC5CC(CC(C5)C3)C4)nc(NCCCNC3Cc4ccccc4C3)n2[nH]1] | 8 | 8 | 8 |
| 102 | 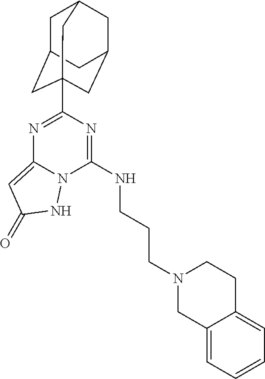 [SMILES: O=c1cc2nc(C34CC5CC(CC(C5)C3)C4)nc(NCCCN3CCc4ccccc4C3)n2[nH]1] | 64 | — | 16 |
| 103 | 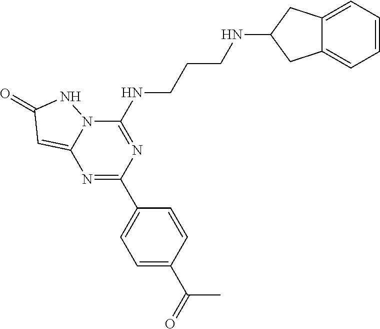 [SMILES: CC(=O)c1ccc(-c2nc(NCCCNC3Cc4ccccc4C3)n3[nH]c(=O)cc3n2)cc1] | 16 | 8 | 32 |
| 104 | 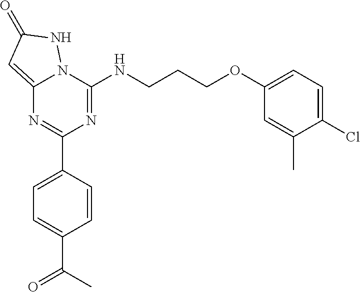 [SMILES: CC(=O)c1ccc(-c2nc(NCCCOc3ccc(Cl)c(C)c3)n3[nH]c(=O)cc3n2)cc1] | 32 | 32 | 32 |
| 105 | 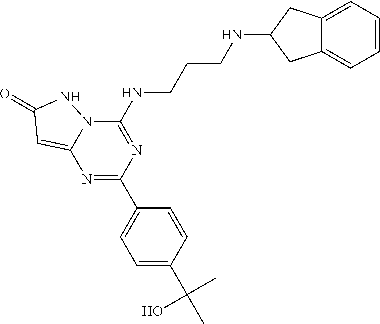 [SMILES: CC(C)(O)c1ccc(-c2nc(NCCCNC3Cc4ccccc4C3)n3[nH]c(=O)cc3n2)cc1] | 32 | 16 | 32 |
| 106 | 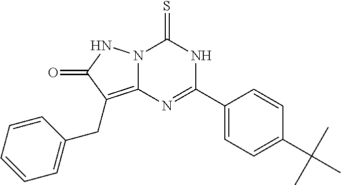 [SMILES: CC(C)(C)c1ccc(-c2nc3c(Cc4ccccc4)c(=O)[nH]n3c(=S)[nH]2)cc1] | 2 | 2 | 1 |
| 107 | 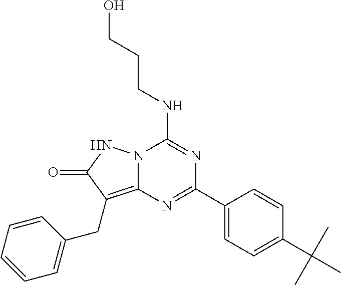 [SMILES: CC(C)(C)c1ccc(-c2nc(NCCCO)n3[nH]c(=O)c(Cc4ccccc4)c3n2)cc1] | >128 | >128 | 64 |
| 108 | 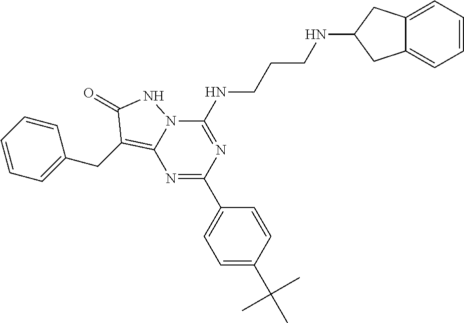 [SMILES: CC(C)(C)c1ccc(-c2nc(NCCCNC3Cc4ccccc4C3)n3[nH]c(=O)c(Cc4ccccc4)c3n2)cc1] | 0.5 | — | 2 |
| 109 | 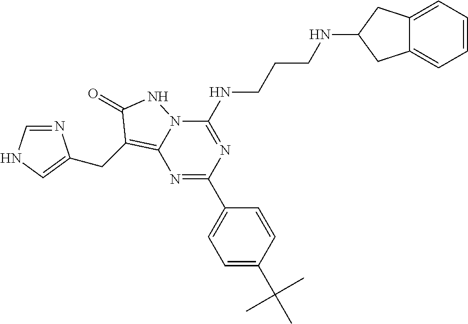 [SMILES: CC(C)(C)c1ccc(-c2nc(NCCCNC3Cc4ccccc4C3)n3[nH]c(=O)c(Cc4c[nH]cn4)c3n2)cc1] | 4 | — | 1 |
| 110 | 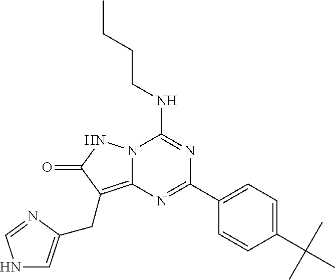 [SMILES: CCCCNc1nc(-c2ccc(C(C)(C)C)cc2)nc2c(Cc3c[nH]cn3)c(=O)[nH]n12] | 4 | — | 2 |
| 111 | 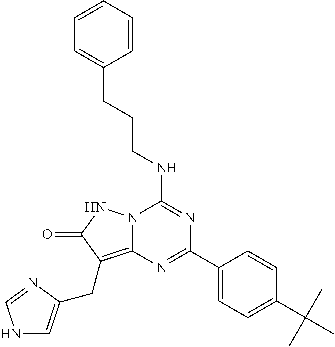 [SMILES: CC(C)(C)c1ccc(-c2nc(NCCCc3ccccc3)n3[nH]c(=O)c(Cc4c[nH]cn4)c3n2)cc1] | 4 | — | 2 |
| 112 | 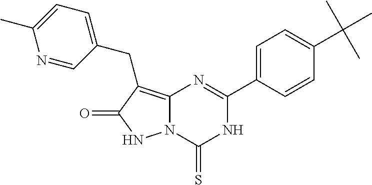 [SMILES: Cc1ccc(Cc2c(=O)[nH]n3c(=S)[nH]c(-c4ccc(C(C)(C)C)cc4)nc23)cn1] | 8 | — | 4 |
| 113 | 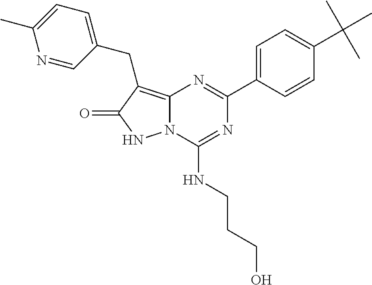 [SMILES: Cc1ccc(Cc2c(=O)[nH]n3c(NCCCO)nc(-c4ccc(C(C)(C)C)cc4)nc23)cn1] | 32 | — | 32 |
| 114 | 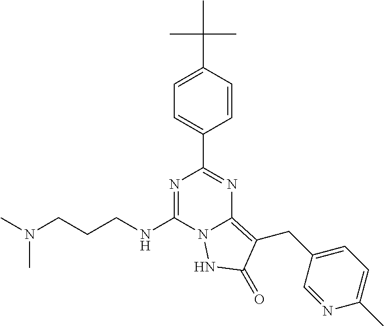 | 64 | — | 64 |
| 115 | 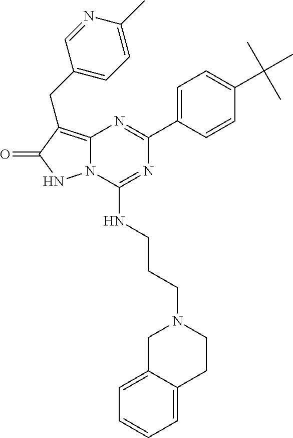 [SMILES: Cc1ccc(Cc2c(=O)[nH]n3c(NCCCN4CCc5ccccc5C4)nc(-c4ccc(C(C)(C)C)cc4)nc23)cn1] | 32 | — | 8 |
| 116 | 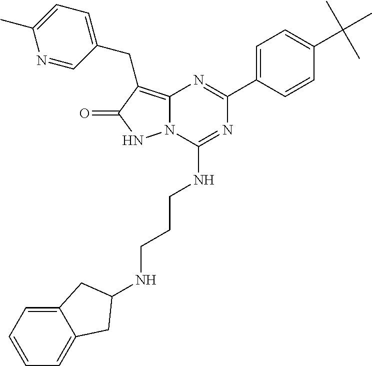 [SMILES: Cc1ccc(Cc2c(=O)[nH]n3c(NCCCNC4Cc5ccccc5C4)nc(-c4ccc(C(C)(C)C)cc4)nc23)cn1] | 4 | — | 4 |
| 117 | 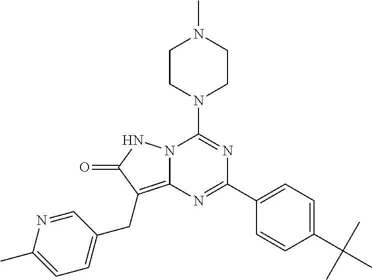 [SMILES: Cc1ccc(Cc2c(=O)[nH]n3c(N4CCN(C)CC4)nc(-c4ccc(C(C)(C)C)cc4)nc23)cn1] | 128 | — | 128 |
| 118 | 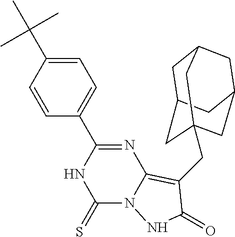 [SMILES: CC(C)(C)c1ccc(-c2nc3c(CC45CC6CC(CC(C6)C4)C5)c(=O)[nH]n3c(=S)[nH]2)cc1] | 8 | — | 0.5 |
| 119 | 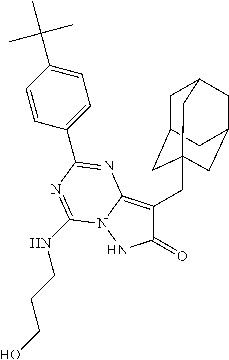 [SMILES: CC(C)(C)c1ccc(-c2nc(NCCCO)n3[nH]c(=O)c(CC45CC6CC(CC(C6)C4)C5)c3n2)cc1] | 16 | — | 16 |
| 120 | 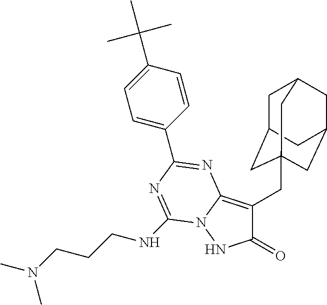 [SMILES: CN(C)CCCNc1nc(-c2ccc(C(C)(C)C)cc2)nc2c(CC34CC5CC(CC(C5)C3)C4)c(=O)[nH]n12] | 1 | — | 1 |
| 121 | 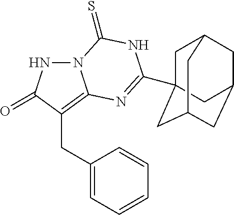 [SMILES: O=c1[nH]n2c(=S)[nH]c(C34CC5CC(CC(C5)C3)C4)nc2c1Cc1ccccc1] | 4 | — | 4 |
| 122 | 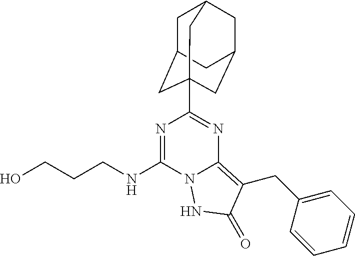 [SMILES: O=c1[nH]n2c(NCCCO)nc(C34CC5CC(CC(C5)C3)C4)nc2c1Cc1ccccc1] | 8 | — | 8 |
| 123 | 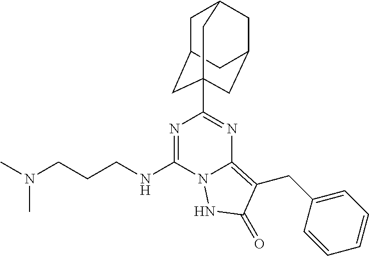 [SMILES: CN(C)CCCNc1nc(C23CC4CC(CC(C4)C2)C3)nc2c(Cc3ccccc3)c(=O)[nH]n12] | 4 | — | 4 |
| 124 | 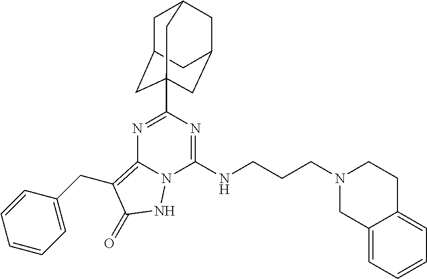 [SMILES: O=c1[nH]n2c(NCCCN3CCc4ccccc4C3)nc(C34CC5CC(CC(C5)C3)C4)nc2c1Cc1ccccc1] | 128 | — | 2 |
| 125 | 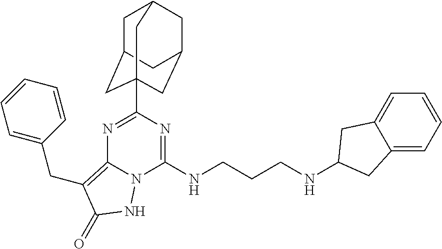 [SMILES: O=c1[nH]n2c(NCCCNC3Cc4ccccc4C3)nc(C34CC5CC(CC(C5)C3)C4)nc2c1Cc1ccccc1] | 2 | — | 1 |

Table 4 shows the MIC values of Exemplified compounds (H. influenzae ATCC 49247).

TABLE 4
MIC values of Exemplified compounds (H. influenzae ATCC 49247).

| | | H. influenzae ATCC |
|---|---|---|
| Example No. | Compound Structure | 49247 MIC μg/mL |
| 19 | 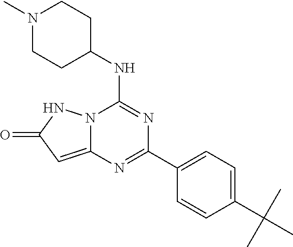 [SMILES: CN1CCC(Nc2nc(-c3ccc(C(C)(C)C)cc3)nc3cc(=O)[nH]n23)CC1] | 64 |
| 31 | 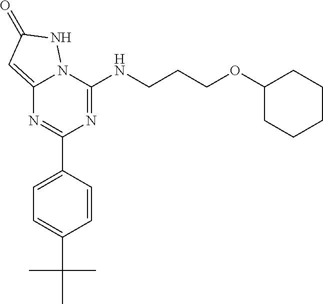 [SMILES: CC(C)(C)c1ccc(-c2nc(NCCCOC3CCCCC3)n3[nH]c(=O)cc3n2)cc1] | 32 |
| 90 | 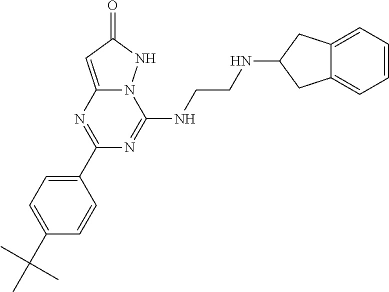 [SMILES: CC(C)(C)c1ccc(-c2nc(NCCNC3Cc4ccccc4C3)n3[nH]c(=O)cc3n2)cc1] | 16 |
| 112 | 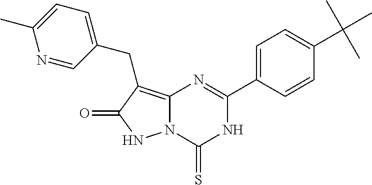 [SMILES: Cc1ccc(Cc2c(=O)[nH]n3c(=S)[nH]c(-c4ccc(C(C)(C)C)cc4)nc23)cn1] | 16 |
| 121 | 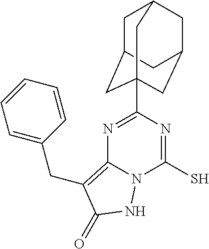 [SMILES: O=c1[nH]n2c(S)nc(C34CC5CC(CC(C5)C3)C4)nc2c1Cc1ccccc1] | 8 |
| 126 | 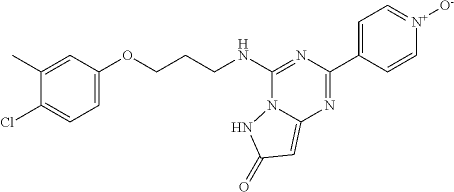 [SMILES: Cc1cc(OCCCNc2nc(-c3cc[n+]([O-])cc3)nc3cc(=O)[nH]n23)ccc1Cl] | 1 |

### V. Luminescence Reporter Assay

S. aureus RN4220 strains harboring pXEN plasmids with a P_{CwrA}-lux construct, termed P1-reporter strain, was used in the experiment. A culture of P1-reporter strain was grown up at 37° C. in chloramphenicol-complemented (10 μg/ml) TSB medium for 16-18 hours. The overnight culture was diluted to OD₆₀₀=0.1 and added (150 μL) to a sterile, black and optically clear bottom 96-well plate (PerkinElmer). The bacterial cultures were incubated in a 37° C. shaker for 30 min before addition of test compounds. A serial dilution of the test compounds was prepared (1 mg/ml, 0.5 mg/ml, 0.25 mg/ml and 0.125 mg/ml) in DMSO, and 1.5 μL of each dilution was added to the 150 μL P1-reporter strain. Moenomycin A (0.009 g/mL) and kanamycin (5 μg/mL) were used as positive and negative controls, respectively. The plate was incubated at 37° C. in a shaker, and the OD₆₀₀ and luminescence signals of P1-reporter strain were monitored using Promega microplate reader every hour. The normalized luminescence (raw luminescence readings divided by OD₆₀₀) data was obtained and plotted as a function of time. Controls of S. aureus RN4220 harboring empty pXEN plasmids was also performed in the luminescence assay, and no significant signals were detected.

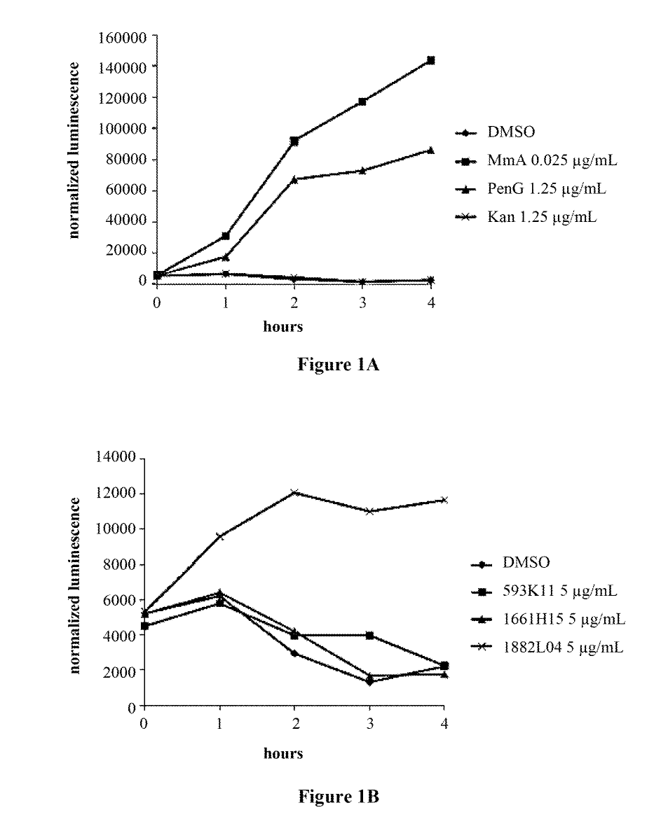

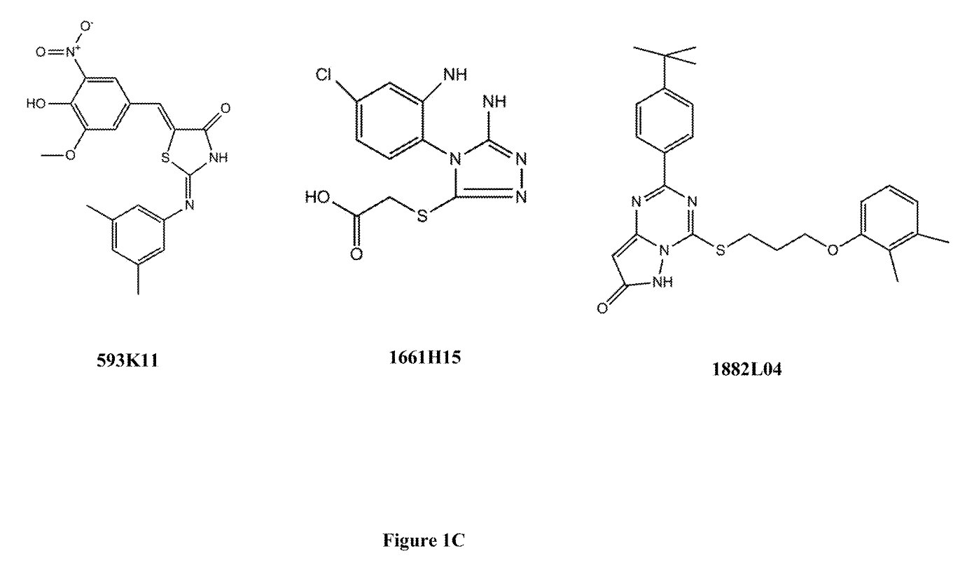

FIG. 1 shows the luminescence reporter assay of antibiotics: Moenomycin (MmA), Penicillin G (PenG), and Kanamycin (Kan) as control and three compounds (593K11, 1661H15, and 1882L04) in the luminescence reporter assay. A comparison of normalized luminescence signals after 4 h for the exemplified compounds are also shown in FIG. 2B.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims.

Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of Formula (I′): 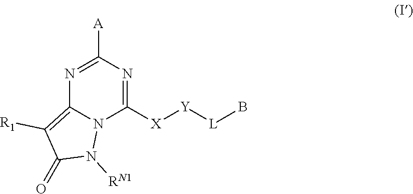
or a pharmaceutically acceptable salt, solvate, hydrate, polymorph, co-crystal, tautomer, stereoisomer, isotopically labeled derivative, or prodrug hereof,
wherein
A is independently optionally substituted C₂₋₆ alkyl, optionally substituted aryl, optionally substituted carbocyclyl, optionally substituted 5-membered heteroaryl, or optionally substituted 6-membered heteroaryl;
B is independently hydrogen, optionally substituted alkyl, optionally substituted alkenyl, optionally substituted alkynyl, optionally substituted aryl, optionally substituted carbocyclyl, optionally substituted heterocyclyl, or optionally substituted heteroaryl;
Y is a bond, optionally substituted C₁₋₆ alkylene, optionally substituted C₃₋₆ carbocyclylene, or optionally substituted heterocyclylene;
X is a bond, —O—, —CH₂—, —NR^{NX}—, —NR^{NX}—C(═O)—NR^{NX}—, or optionally substituted heterocyclylene;
L is a bond, —O—, —C(═O)—, —NR^{LB}C(═O)—, —C(═O)NR^{LB}—, —NR^{LB}—, or —SO₂—;
each instance of R^{LB} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or R^{LB} and B are taken together with their intervening atoms to form an optionally substituted heterocyclic ring;
R₁ is hydrogen, halogen, or optionally substituted C₁₋₆ alkyl;
R^{N1} is hydrogen, optionally substituted C₁₋₆ alkyl, or a nitrogen protecting group; and
R^{NX} is hydrogen, optionally substituted C₁₋₆ alkyl, or a nitrogen protecting group;
provided that the compound of Formula (I′) is not of the formula:

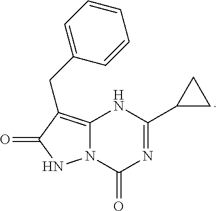 [SMILES: O=c1[nH]n2c(=O)nc(C3CC3)[nH]c2c1Cc1ccccc1]

2. A compound of Formula (I): 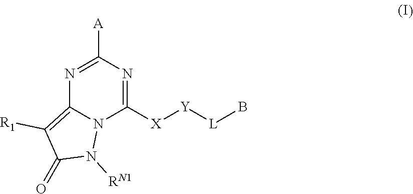
or a pharmaceutically acceptable salt thereof,
wherein
A is independently optionally substituted aryl, optionally substituted 5-membered heteroaryl, or optionally substituted 6-membered heteroaryl;
B is independently hydrogen, optionally substituted alkyl, optionally substituted alkenyl, optionally substituted alkynyl, optionally substituted aryl, optionally substituted carbocyclyl, optionally substituted heterocyclyl, or optionally substituted heteroaryl;
Y is a bond, optionally substituted C₁₋₆ alkylene, optionally substituted C₃₋₆ carbocyclylene, or optionally substituted heterocyclylene;
X is a bond, —O—, —S—, —CH₂—, —NR^{NX}—, —NR^{NX}—C(═O)—NR^{NX}—, or optionally substituted heterocyclylene;
L is a bond, —O—, —C(═O)—, —NR^{LB}C(═O)—, —C(═O)NR^{LB}—, —NR^{LB}—, or —SO₂—;
each instance of R^{LB} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, or R^{LB} and B are taken together with their intervening atoms to form an optionally substituted heterocyclic ring;
R₁ is hydrogen, halogen, or optionally substituted C₁₋₆ alkyl;
R^{N1} is hydrogen, optionally substituted C₁₋₆ alkyl, or a nitrogen protecting group; and
R^{NX} is hydrogen, optionally substituted C₁₋₆ alkyl, or a nitrogen protecting group.

3. A compound of any one of claims 1-2, wherein the compound is of Formula (I-i): 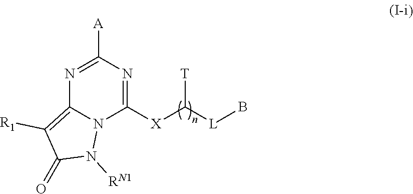
or a pharmaceutically acceptable salt, solvate, hydrate, polymorph, co-crystal, tautomer, stereoisomer, isotopically labeled derivative, or prodrug thereof,
wherein
each instance of T is independently hydrogen, halogen, optionally substituted C₁₋₆ alkyl, or —OR^{T};
each instance of R^{T} is independently hydrogen, optionally substituted C₁₋₆ alkyl, or an oxygen protecting group; and
n is 0 or an integer of 1 to 6, inclusive.

4. A compound of any one of claims 1-3, wherein the compound is of Formula (I′-i-A): 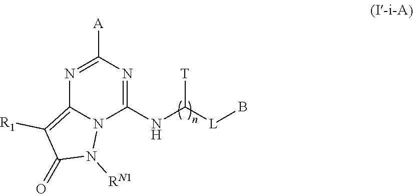
or a pharmaceutically acceptable salt, solvate, hydrate, polymorph, co-crystal, tautomer, stereoisomer, isotopically labeled derivative, or prodrug thereof.

5. A compound of any one of claims 1-3, wherein the compound is of Formula (I-ii): 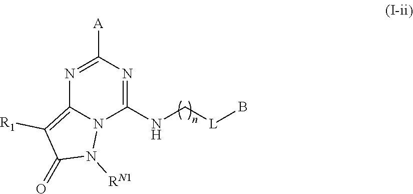
or a pharmaceutically acceptable salt, solvate, hydrate, polymorph, co-crystal, tautomer, stereoisomer, isotopically labeled derivative, or prodrug thereof.

6. A compound of any one of claims 1-2, wherein the compound is of Formula (I-ii-A): 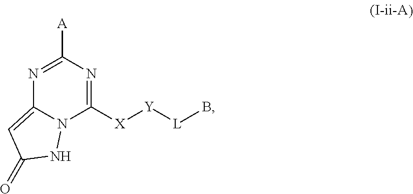
or a pharmaceutically acceptable salt, solvate, hydrate, polymorph, co-crystal, tautomer, stereoisomer, isotopically labeled derivative, or prodrug thereof.

7. A compound of Formula (IA): 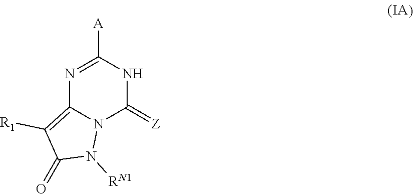
or pharmaceutically acceptable salt, solvate, hydrate, polymorph, co-crystal, tautomer, stereoisomer, isotopically labeled derivative, or prodrug thereof,
wherein
A is independently optionally substituted C₂₋₆ alkyl, optionally substituted aryl, optionally substituted C₄₋₁₀ carbocyclyl, optionally substituted 5-membered heteroaryl, or optionally substituted 6-membered heteroaryl;
Z is S or O;
R₁ is hydrogen, halogen, or optionally substituted C₁₋₆ alkyl;
R^{N1} is hydrogen, optionally substituted C₁₋₆ alkyl, or a nitrogen protecting group; and
R^{NX} is hydrogen, optionally substituted C₁₋₆ alkyl, or a nitrogen protecting group;
provided that the compound of Formula (IA) is not of the formula:

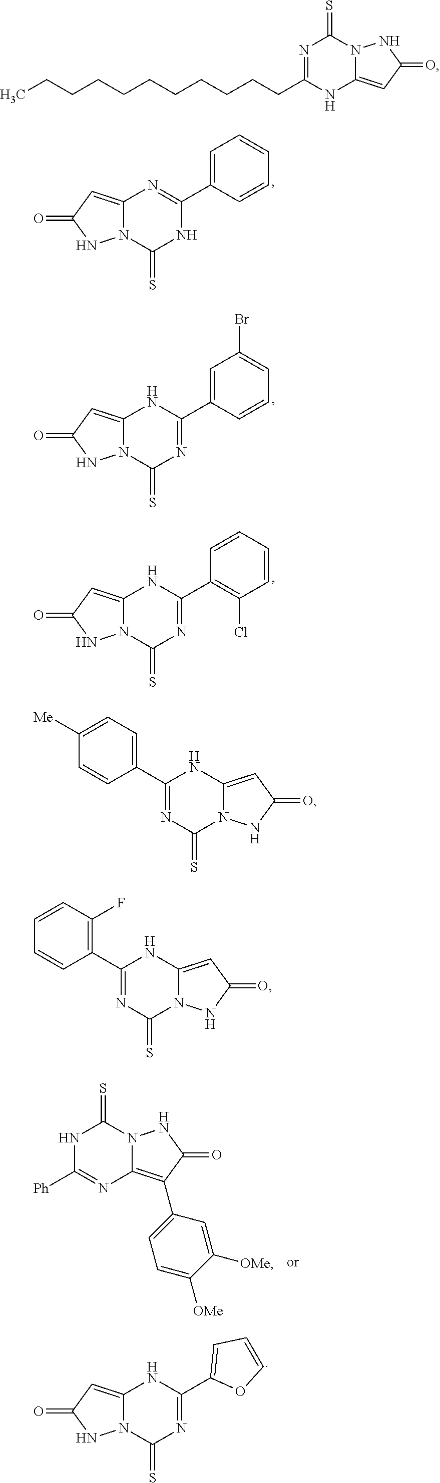

8. The compound of claim 7, wherein the compound is of the formula: 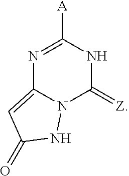
or a pharmaceutically acceptable salt, solvate, hydrate, polymorph, co-crystal, tautomer, stereoisomer, isotopically labeled derivative, or prodrug thereof.

9. The compound of any one of claims 1-6, wherein:
A is independently unsubstituted C₂₋₆ alkyl, unsubstituted aryl, unsubstituted carbocyclyl, unsubstituted 5-membered heteroaryl, or unsubstituted 6-membered heteroaryl;
B is independently hydrogen, unsubstituted alkyl, unsubstituted alkenyl, unsubstituted alkynyl, unsubstituted aryl, unsubstituted carbocyclyl, unsubstituted heterocyclyl, or unsubstituted heteroaryl;
Y is a bond, unsubstituted C₁₋₆ alkylene, unsubstituted C₃₋₆ carbocyclylene, or unsubstituted heterocyclylene;
X is a bond, —O—, —CH₂—, —NR^{NX}—, —NR^{NX}—C(═O)—NR^{NX}—, or unsubstituted heterocyclylene;
L is a bond, —O—, —C(═O)—, —NR^{LB}C(═O)—, —C(═O)NR^{LB}—, —NR^{LB}—, or —SO₂—;
each instance of R^{LB} is independently selected from the group consisting of hydrogen, unsubstituted alkyl, unsubstituted alkenyl, unsubstituted alkynyl, unsubstituted carbocyclyl, unsubstituted heterocyclyl, unsubstituted aryl, and unsubstituted heteroaryl, or R^{LB} and B are taken together with their intervening atoms to form unsubstituted heterocyclic ring;
R₁ is hydrogen, halogen, or unsubstituted C₁₋₆ alkyl;
R^{N1} is hydrogen, unsubstituted C₁₋₆ alkyl, or a nitrogen protecting group; and
R^{NX} is hydrogen, unsubstituted C₁₋₆ alkyl, or a nitrogen protecting group.

10. The compound of any one of claims 1-6 or claim 9, wherein X is —NR^{NX}—, Y is a bond, L is a bond, and B is hydrogen.

11. The compound of any one of claims 1-6, wherein X is —NR^{NX}—, Y is optionally substituted C₁₋₆ alkylene, L is —O—, and B is hydrogen, optionally substituted alkyl, optionally substituted carbocyclyl, optionally substituted aryl, or optionally substituted heteroaryl.

12. The compound of any one of claims 1-6, wherein X is —NR^{NX}—, Y is optionally substituted C₁₋₆ alkylene, L is —NR^{LB}—, and R^{LB} and B are taken together with their intervening atoms to form an optionally substituted heterocyclic ring.

13. The compound of any one of claims 1-6, wherein X is —NR^{NX}—, Y is optionally substituted C₁₋₆ alkylene, L is —NR^{LB}—, and B is optionally substituted alkyl, optionally substituted carbocyclyl, or optionally substituted aryl.

14. The compound of any one of claims 1-6, wherein X is —NR^{NX}—, Y is optionally substituted C₁₋₆ alkylene, L is —SO₂—, and B is optionally substituted aryl.

15. The compound of any one of claims 1-6, wherein X is —NR^{NX}—, Y is optionally substituted C₁₋₆ alkylene, L is a bond, and B is hydrogen, optionally substituted alkyl, optionally substituted heterocyclyl, optionally substituted aryl, or optionally substituted heteroaryl.

16. The compound of any one of claims 1, 2, or 6, wherein X is —NR^{NX}—, Y is optionally substituted carbocyclylene, L is a bond or —NR^{LB}—, and B is optionally substituted alkyl.

17. The compound of any one of claims 1, 2, or 6, wherein X is —NR^{NX}—, Y is optionally substituted heterocyclylene, L is a bond, and B is optionally substituted alkyl, optionally substituted aryl, or optionally substituted heteroaryl.

18. The compound of any one of claims 1, 2, or 6, wherein X is —NR^{NX}—, Y is optionally substituted heterocyclylene, L is —C(═O)—, and B is optionally substituted heteroaryl or aryl.

19. The compound of any one of claims 1-3, 5, or 6, wherein X is O, Y is a bond, L is a bond, and B is hydrogen.

20. The compound of any one of claims 1-3, 5, or 6, wherein X is S, Y is a bond, L is a bond, and B is hydrogen.

21. The compound of any one of claims 1-3, 5, or 6, wherein X is optionally substituted heterocyclene, Y is a bond, L is a bond, and B is hydrogen or optionally substituted aryl.

22. The compound of any one of claims 1-3, 5, or 6, wherein X is optionally substituted heterocyclene, Y is a bond, L is —NR^{LB}—, and B is hydrogen or optionally substituted aryl.

23. The compound of any one of claims 1-3, 5, or 6, wherein X is optionally substituted heterocyclene, Y is optionally substituted C₁₋₆ alkylene, L is —NR^{LB}—, and B is optionally substituted alkyl or aryl.

24. The compound of any one of claims 1-3, 5, or 6, wherein X is optionally substituted heterocyclene, Y is optionally substituted C₁₋₆ alkylene, L is —O—, and B is hydrogen or optionally substituted alkyl.

25. The compound of any one of claims 1-3, 5, or 6, wherein X is optionally substituted heterocyclene, Y is a optionally substituted C₁₋₆ alkylene, L is a bond, and B is hydrogen.

26. The compound of any one of claims 1-3, 5, or 6, wherein X is optionally substituted heterocyclene, Y is a bond, L is —O—, and B is hydrogen or optionally substituted alkyl.

27. The compound of any one of claims 7-8, wherein Z is S.

28. The compound of any one of claims 7-8, wherein Z is O.

29. The compound of any one of claims 1-28, wherein A is optionally substituted C₂₋₆ alkyl.

30. The compound of any one of claims 1-29, wherein A is of the formula:

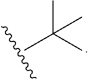

31. The compound of any one of claims 1-28, wherein A is optionally substituted aryl.

32. The compound of any one of claims 1-28 or 31, wherein A is optionally substituted phenyl.

33. The compound of any one of claims 1-28 or 31-32, wherein A is substituted phenyl.

34. The compound of any one of claims 1-28 or 31-33, wherein A is of the formula:

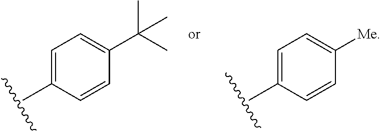

35. The compound of any one of claims 1-28 or 31-32, wherein A is unsubstituted phenyl.

36. The compound of any one of claims 1-28, wherein A is optionally substituted C₄₋₁₀ carbocyclyl.

37. The compound of any one of claims 1-28 or 36, wherein A is of the formula:

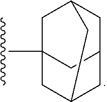

38. The compound of any one of claims 1-28, wherein A is optionally substituted furanyl or thiophenyl.

39. The compound of claim 38, wherein A is of the formula:

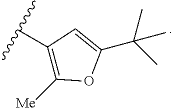 [SMILES: *c1cc(C(C)(C)C)oc1C]

40. The compound of any one of claims 1-6, 11, 13, or 29-39, wherein B is optionally substituted carbocyclyl.

41. The compound of any one of claims 1-6, 11, 13, or 29-40, wherein B is of the formula:

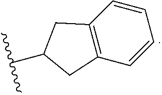 [SMILES: *C1Cc2ccccc2C1]

42. The compound of any one of claims 1-6, 11, 13, 15, 17-18, 21-23, or 29-39, wherein B is optionally substituted aryl.

43. The compound of any one of claims 1-6, 11, 13, 15, 17-18, 21-23, 29-39, or 42, wherein B is optionally substituted monocyclic aryl.

44. The compound of any one of claims 1-6, 11, 13, 15, 17-18, 21-23, 29-39, or 42-43, wherein B is optionally substituted phenyl.

45. The compound of claim 44, wherein B is of the formula:

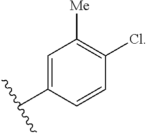 [SMILES: *c1ccc(Cl)c(C)c1]

46. The compound of any one of claims 1-6, 11, 15, 17-18, or 29-39, wherein B is optionally substituted five-membered heteroaryl.

47. The compound of any one of claims 1-6, 11, 15, 17-18, or 29-39, wherein B is optionally substituted six-membered heteroaryl.

48. The compound of any one of claims 1-6, 9, 11, 15, 21-22, 24, 26, or 29-39, wherein B is hydrogen.

49. The compound of any one of claims 1-6, 11, 13, 15, 17, 23-24, 26, or 29-39, wherein B is optionally substituted alkyl.

50. The compound of claim 49, wherein B is optionally substituted C₁₋₆ alkyl.

51. The compound of claim 50, wherein B is methyl.

52. A compound of claim 3, wherein the compound is of Formula (II): 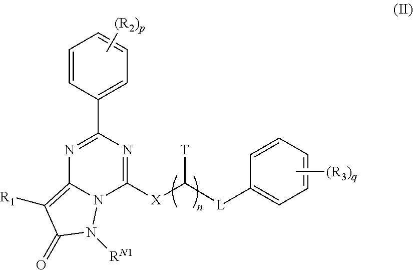
or a pharmaceutically acceptable salt, solvate, hydrate, polymorph, co-crystal, tautomer, stereoisomer, isotopically labeled derivative, or prodrug thereof,
wherein:
each instance of R₂ is independently selected from the group consisting of hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(═O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, and —SO₂N(R^{B})₂;
each instance of R₃ is independently selected from the group consisting of hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, and —SO₂N(R^{B})₂;
each instance of R^{A} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and an oxygen protecting group;
each instance of R^{B} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group, or two R^{B} groups are taken together with their intervening atoms to form an optionally substituted heterocyclic ring;
p is independently 0, 1, 2, 3, 4, or 5; and
q is independently 0, 1, 2, 3, 4, or 5.

53. A compound of claim 52, wherein the compound is Formula (II-i): 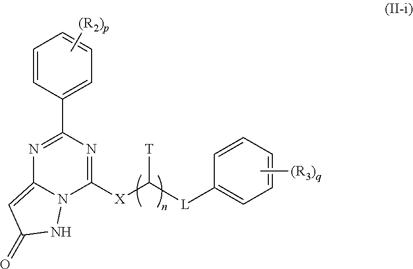
or a pharmaceutically acceptable salt, solvate, hydrate, polymorph, co-crystal, tautomer, stereoisomer, isotopically labeled derivative, or prodrug thereof.

54. The compound of claim 52, wherein:
each instance of R₂ is independently selected from the group consisting of hydrogen, halogen, —CN, —NO₂, —N₃, unsubstituted alkyl, unsubstituted alkenyl, unsubstituted alkynyl, unsubstituted carbocyclyl, unsubstituted aryl, unsubstituted heterocyclyl, unsubstituted heteroaryl, —OR^{A}, —N(R^{B})₂, —SR^{A}, —C(═O)R^{A}, —C(═O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, and —SO₂N(R^{B})₂;
each instance of R₃ is independently selected from the group consisting of hydrogen, halogen, —CN, —NO₂, —N₃, unsubstituted alkyl, unsubstituted alkenyl, unsubstituted alkynyl, unsubstituted carbocyclyl, unsubstituted aryl, unsubstituted heterocyclyl, unsubstituted heteroaryl, —OR^{A}, —N(R^{B})₂, —SR^{A}, —C(═O)R^{A}, —C(O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, and —SO₂N(R^{B})₂;
each instance of R^{A} is independently selected from the group consisting of hydrogen, unsubstituted alkyl, unsubstituted alkenyl, unsubstituted alkynyl, unsubstituted carbocyclyl, unsubstituted heterocyclyl, unsubstituted aryl, unsubstituted heteroaryl, and an oxygen protecting group;
each instance of R^{B} is independently selected from the group consisting of hydrogen, unsubstituted alkyl, unsubstituted alkenyl, unsubstituted alkynyl, unsubstituted carbocyclyl, unsubstituted heterocyclyl, unsubstituted aryl, unsubstituted heteroaryl, and a nitrogen protecting group, or two R^{B} groups are taken together with their intervening atoms to form an unsubstituted heterocyclic ring.

55. The compound of claim 52, wherein the compound is of Formula (II-b): 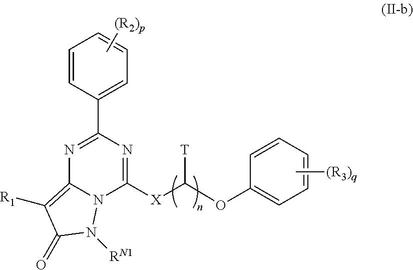
or a pharmaceutically acceptable salt, solvate, hydrate, polymorph, co-crystal, tautomer, stereoisomer, isotopically labeled derivative, or prodrug thereof.

56. The compound of claim 52, wherein the compound is of Formula (II-c): 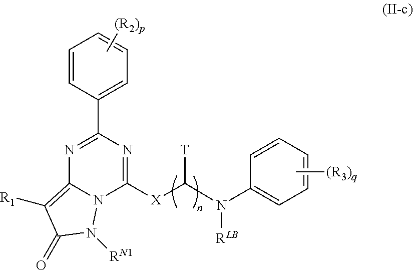
or a pharmaceutically acceptable salt, solvate, hydrate, polymorph, co-crystal, tautomer, stereoisomer, isotopically labeled derivative, or prodrug thereof.

57. The compound of any one of claims 1, 2, or 56, wherein R^{LB} is H.

58. A compound of claim 3, wherein the compound is of Formula (IV): 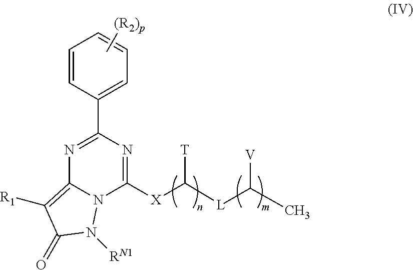
or a pharmaceutically acceptable salt, solvate, hydrate, polymorph, co-crystal, tautomer, stereoisomer, isotopically labeled derivative, or prodrug thereof,
wherein:
each instance of V is independently hydrogen, halogen, optionally substituted C₁₋₆ alkyl, optionally substituted alkenyl, optionally substituted alkynyl, optionally substituted carbocyclyl, optionally substituted aryl, optionally substituted heterocyclyl, optionally substituted heteroaryl, —N(R^{V1})₂, or —OR^{V2};
each instance of R₂ is independently selected from the group consisting of hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(═O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, and —SO₂N(R^{B})₂;
each instance of R^{A} and R^{V2} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and an oxygen protecting group;
each instance of R^{B} and R^{V1} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group, or two R^{B} groups are taken together with their intervening atoms to form an optionally substituted heterocyclic ring, or two R^{V1} groups are taken together with their intervening atoms to form an optionally substituted heterocyclic ring;
p is independently 0, 1, 2, 3, 4, or 5; and
m is independently 0, 1, 2, 3, 4, 5, or 6.

59. A compound of claim 58, wherein the compound is of Formula (IV-i): 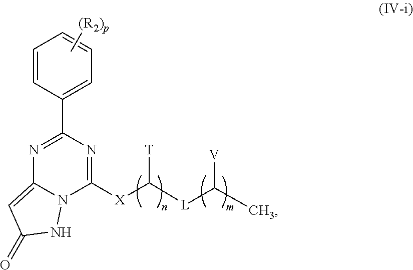
or a pharmaceutically acceptable salt, solvate, hydrate, polymorph, co-crystal, tautomer, stereoisomer, isotopically labeled derivative, or prodrug thereof.

60. The compound of claim 59, wherein
each instance of V is independently hydrogen, halogen, unsubstituted C₁₋₆ alkyl, unsubstituted alkenyl, unsubstituted alkynyl, unsubstituted carbocyclyl, unsubstituted aryl, unsubstituted heterocyclyl, unsubstituted heteroaryl, —N(R^{V1})₂, or —OR^{V2};
each instance of R₂ is independently selected from the group consisting of hydrogen, halogen, —CN, —NO₂, —N₃, unsubstituted alkyl, unsubstituted alkenyl, unsubstituted alkynyl, unsubstituted carbocyclyl, unsubstituted aryl, unsubstituted heterocyclyl, unsubstituted heteroaryl, —OR^{A}, —N(R^{B})₂, —SR^{A}, —C(═O)R^{A}, —C(═O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, and —SO₂N(R^{B});
each instance of R^{A} and R^{V2} is independently selected from the group consisting of hydrogen, unsubstituted alkyl, unsubstituted alkenyl, unsubstituted alkynyl, unsubstituted carbocyclyl, unsubstituted aryl, unsubstituted heterocyclyl, unsubstituted heteroaryl, and an oxygen protecting group; and
each instance of R^{B} and R^{V1} is independently selected from the group consisting of hydrogen, unsubstituted alkyl, unsubstituted alkenyl, unsubstituted alkynyl, unsubstituted carbocyclyl, unsubstituted aryl, unsubstituted heterocyclyl, unsubstituted heteroaryl, and a nitrogen protecting group, or two R^{B} groups are taken together with their intervening atoms to form an unsubstituted heterocyclic ring, or two R^{V1} groups are taken together with their intervening atoms to form an unsubstituted heterocyclic ring.

61. The compound of claim 58, wherein the compound is of Formula (IV-a): 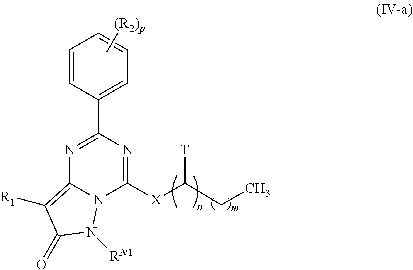
or a pharmaceutically acceptable salt, solvate, hydrate, polymorph, co-crystal, tautomer, stereoisomer, isotopically labeled derivative, or prodrug thereof.

62. The compound of claim 58, wherein the compound is of Formula (IV-b): 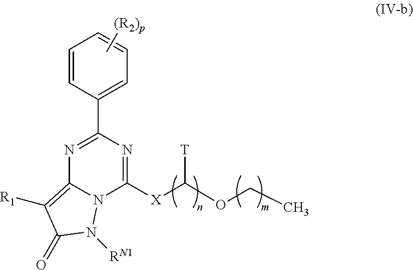
or a pharmaceutically acceptable salt, solvate, hydrate, polymorph, co-crystal, tautomer, stereoisomer, isotopically labeled derivative, or prodrug thereof.

63. The compound of claim 58, wherein the compound is of Formula (IV-d): 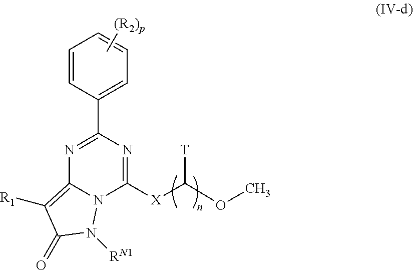
or a pharmaceutically acceptable salt, solvate, hydrate, polymorph, co-crystal, tautomer, stereoisomer, isotopically labeled derivative, or prodrug thereof.

64. A compound of claim 3, wherein the compound is of Formula (VI): 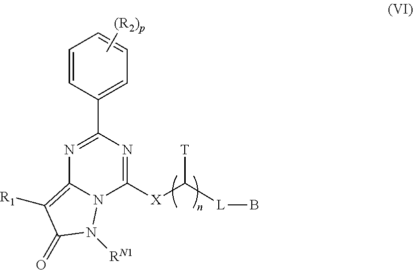
or a pharmaceutically acceptable salt, solvate, hydrate, polymorph, co-crystal, tautomer, stereoisomer, isotopically labeled derivative, or prodrug thereof.

65. A compound of claim 64, wherein the compound is of Formula (VI-1-a): 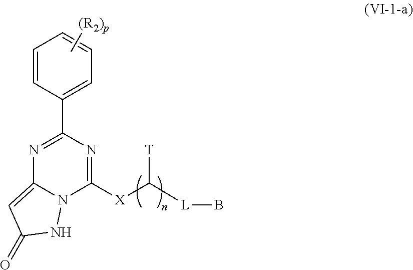
or a pharmaceutically acceptable salt, solvate, hydrate, polymorph, co-crystal, tautomer, stereoisomer, isotopically labeled derivative, or prodrug thereof.

66. A compound of any one of claims 64-65, wherein B is an optionally substituted bicyclic heterocyclyl.

67. A compound of claim 64, wherein the compound is of Formula (VI-m): 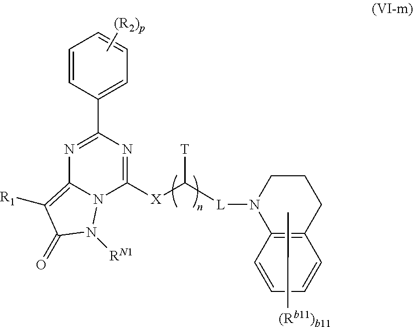
wherein
each instance of R^{b11} is independently selected from the group consisting of hydrogen, halogen, —CN, —NO₂, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, or —SO₂N(R^{B})₂;
each instance of R^{A} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, and an oxygen protecting group;
each instance of R^{B} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, and a nitrogen protecting group, or two R^{B} groups are taken together with their intervening atoms to form an optionally substituted heterocyclic ring; and
b11 is independently 0, 1, 2, 3, 4, 5, 6, 7, 8, 9, or 10.

68. A compound of claim 64, wherein the compound is of Formula (VI-n): 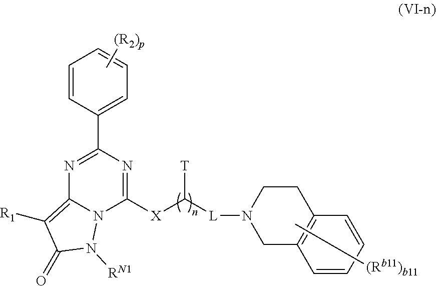
wherein
each instance of R^{b11} is independently selected from the group consisting of hydrogen, halogen, —CN, —NO₂, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, or —SO₂N(R^{B})₂;
each instance of R^{A} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, and an oxygen protecting group;
each instance of R^{B} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, and a nitrogen protecting group, or two R^{B} groups are taken together with their intervening atoms to form an optionally substituted heterocyclic ring; and
b11 is independently 0, 1, 2, 3, 4, 5, 6, 7, 8, 9, or 10.

69. A compound of claim 64, wherein B is an optionally substituted monocyclic carbocyclyl.

70. A compound of claim 69, wherein B is an optionally substituted 6-membered monocyclic carbocyclyl.

71. A compound of claim 64, wherein the compound is of Formula (VI-j): 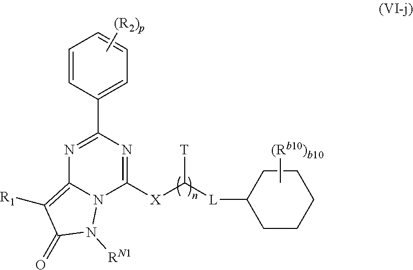
wherein
each instance of R^{b10} is independently selected from the group consisting of hydrogen, halogen, —CN, —NO₂, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, or —SO₂N(R^{B})₂;
each instance of R^{A} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, and an oxygen protecting group;
each instance of R^{B} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, and a nitrogen protecting group, or two R^{B} groups are taken together with their intervening atoms to form an optionally substituted heterocyclic ring; and
b10 is independently 0 or an integer of 1 to 10, inclusive.

72. A compound of claim 71, wherein at least one instance of R^{b10} is hydrogen.

73. A compound of claim 64, wherein B is an optionally substituted bicyclic carbocyclyl.

74. A compound of claim 64, wherein the compound is of Formula (VI-k): 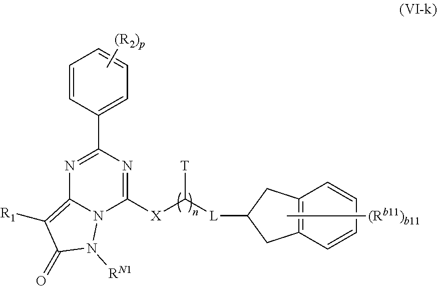
wherein
each instance of R^{b11} is independently selected from the group consisting of hydrogen, halogen, —CN, —NO₂, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, or —SO₂N(R^{B})₂;
each instance of R^{A} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, and an oxygen protecting group;
each instance of R^{B} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, and a nitrogen protecting group, or two R^{B} groups are taken together with their intervening atoms to form an optionally substituted heterocyclic ring; and
b11 is independently 0, 1, 2, 3, 4, 5, 6, 7, or 8.

75. A compound of claim 74, wherein at least one instance of R^{b11} is hydrogen.

76. A compound of claim 64, wherein B is hydrogen.

77. The compound of any one of claims 1-6, wherein Y is a bond.

78. The compound of any one of claims 1-6, wherein Y is optionally substituted C₁₋₆ alkylene.

79. The compound of any one of claims 1-6 or 78, wherein Y is unsubstituted C₁₋₆ alkylene.

80. The compound of any one of claims 1-2, or 6, wherein Y is optionally substituted carbocyclylene.

81. The compound of any one of claims 1-2, 6 or 80, wherein Y is optionally substituted 6-membered carbocyclylene.

82. The compound of any one of claims 1 or 2, wherein the compound is of Formula (VII): 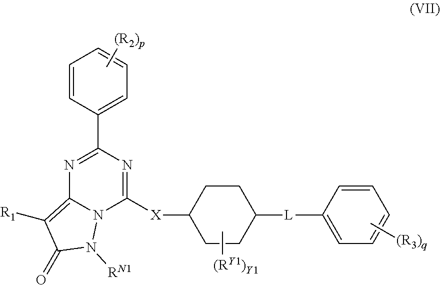
or a pharmaceutically acceptable salt, solvate, hydrate, polymorph, co-crystal, tautomer, stereoisomer, isotopically labeled derivative, or prodrug thereof,
wherein:
each instance of R₂, R₃, and R^{Y1} is independently selected from the group consisting of hydrogen, halogen, —CN, —NO₂, —N₃, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(═O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, and —SO₂N(R^{B})₂;
each instance of R^{A} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and an oxygen protecting group;
each instance of R^{B} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group, or two R^{B} groups are taken together with their intervening atoms to form an optionally substituted heterocyclic ring;
each instance of R₂, R₃, R^{Y1}, R_{A}, or R_{B} is independently optionally substituted with hydrogen, halogen, —CN, —NO₂, —N₃, acyl, alkyl, alkenyl, alkynyl, carbocyclyl, aryl, heterocyclyl, heteroaryl, —OR, —N(R^{Z})₂, or —SR; and R is hydrogen, halogen, —CN, —NO₂, —N₃, acyl, alkyl, alkenyl, alkynyl, carbocyclyl, aryl, heterocyclyl, heteroaryl, oxygen protecting group when attached to an oxygen atom, or sulfur protecting group when attached to an sulfur atom; and R^{Z} is hydrogen, halogen, —CN, —NO₂, —N₃, acyl, alkyl, alkenyl, alkynyl, carbocyclyl, aryl, heterocyclyl, heteroaryl, or nitrogen protecting group;
p is independently 0, 1, 2, 3, 4, or 5;
q is independently 0, 1, 2, 3, 4, or 5; and
Y1 is independently 0, 1, 2, 3, 4, 5, 6, 7, or 8.

83. The compound of claim 82, wherein the compound is of Formula (VII-i): 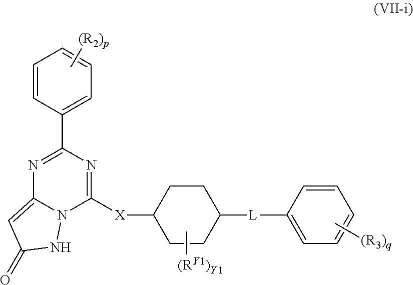
or a pharmaceutically acceptable salt, solvate, hydrate, polymorph, co-crystal, tautomer, stereoisomer, isotopically labeled derivative, or prodrug thereof.

84. The compound of claim 83, wherein
each instance of R₂, R₃, and R^{Y1} is independently selected from the group consisting of hydrogen, halogen, —CN, —NO₂, —N₃, unsubstituted alkyl, unsubstituted alkenyl, unsubstituted alkynyl, unsubstituted carbocyclyl, unsubstituted aryl, unsubstituted heterocyclyl, unsubstituted heteroaryl, —OR^{A}, —N(R^{B})₂, —SR^{A}, —C(═O)R^{A}, —C(═O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, and —SO₂N(R^{B})₂;
each instance of R^{A} is independently selected from the group consisting of hydrogen, unsubstituted alkyl, unsubstituted alkenyl, unsubstituted alkynyl, unsubstituted carbocyclyl, unsubstituted aryl, unsubstituted heterocyclyl, unsubstituted heteroaryl, and an oxygen protecting group;
each instance of R^{B} is independently selected from the group consisting of hydrogen, unsubstituted alkyl, unsubstituted alkenyl, unsubstituted alkynyl, unsubstituted carbocyclyl, unsubstituted aryl, unsubstituted heterocyclyl, unsubstituted heteroaryl, and a nitrogen protecting group, or two R^{B} groups are taken together with their intervening atoms to form an unsubstituted heterocyclic ring.

85. The compound of any one of claims 82-84, wherein at least one instance of R^{Y1} is hydrogen.

86. The compound of any one of claims 1-6, 52-54, 58-60, or 64-85, wherein L is —O—.

87. The compound of any one of claims 1-6, 52-54, 58-60, or 64-85, wherein L is —NR^{LB}—.

88. The compound of any one of claims 1-6, 52-54, 54-56, 58-60, 64-85, or 87, wherein L is —NH—.

89. The compound of any one of claims 1-3, 5-6, or 29-88, wherein X is —NR^{NX}—, —NR^{NX}—C(═O)—NR^{NX}—, or optionally substituted heterocyclylene.

90. The compound of any one of claims 1-3, 5-6, or 29-88, wherein X is —O—.

91. The compound of any one of claims 1-3, 5-6, or 29-88, wherein X is —CH₂—.

92. The compound of any one of claims 1-3, 5-6, or 29-88, wherein X is —NR^{RX}—.

93. The compound of any one of claims 1-3, 5-6, 29-88, or 92, wherein X is —NH—.

94. The compound of any one of claims 1-3, 5-6, or 29-89, wherein X is —NR^{RX}—C(═O)—NR^{RX}—.

95. The compound of any one of claims 1-3, 5-6, 29-89, or 94, wherein X is —NH—C(═O)—NH—.

96. The compound of any one of claims 1-3, 5-6, or 29-88, wherein X is optionally substituted heterocyclylene.

97. The compound of any one of claims 1-3, 5-6, 29-88, or 96, wherein X is of the formula 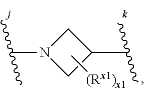
wherein
k indicates the point of attachment to Y;
j indicates the point of attachment to the triazine ring;
each instance of R^{x1} is independently selected from the group consisting of halogen, —CN, —NO₂, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(O)OR, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, or —SO₂N(R^{B})₂;
each instance of R^{A} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, and an oxygen protecting group;
each instance of R^{B} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, and a nitrogen protecting group, or two R^{B} groups are taken together with their intervening atoms to form an optionally substituted heterocyclic ring; and
x1 is independently 0, 1, 2, 3, or 4.

98. The compound of any one of claims 1-3, 5-6, 29-88, or 96, wherein X is of the formula 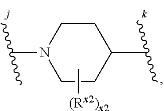
wherein
k indicates the point of attachment to Y;
j indicates the point of attachment to the triazine ring;
each instance of R^{x2} is independently selected from the group consisting of halogen, —CN, —NO₂, optionally substituted alkyl, optionally substituted alkenyl, optionally substituted alkynyl, optionally substituted carbocyclyl, optionally substituted aryl, optionally substituted heterocyclyl, optionally substituted heteroaryl, —OR^{A}, —N(R^{B})₂, —SR^{A}, —C(═O)R^{A}, —C(O)OR^{A}, —C(═O)SR^{A}, —C(═O)N(R^{B})₂, —C(═O)N(R^{B})N(R^{B})₂, —OC(═O)R^{A}, —OC(═O)N(R^{B})₂, —NR^{B}C(═O)R^{A}, —NR^{B}C(═O)N(R^{B})₂, —NR^{B}C(═O)N(R^{B})N(R^{B})₂, —NR^{B}C(═O)OR^{A}, —SC(═O)R^{A}, —C(═NR^{B})R^{A}, —C(═NNR^{B})R^{A}, —C(═NOR^{A})R^{A}, —C(═NR^{B})N(R^{B})₂, —NR^{B}C(═NR^{B})R^{B}, —C(═S)R^{A}, —C(═S)N(R^{B})₂, —NR^{B}C(═S)R^{A}, —S(═O)R^{A}, —OS(═O)₂R^{A}, —SO₂R^{A}, —NR^{B}SO₂R^{A}, or —SO₂N(R^{B})₂;
each instance of R^{A} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, and an oxygen protecting group;
each instance of R^{B} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl, and a nitrogen protecting group, or two R^{B} groups are taken together with their intervening atoms to form an optionally substituted heterocyclic ring; and
x2 is independently 0, or an integer of 1 to 8, inclusive.

99. The compound of any one of claims 7-8 or 29-39 wherein Z is S.

100. The compound of any one of claims 1-99 wherein A is independently selected from the group consisting of

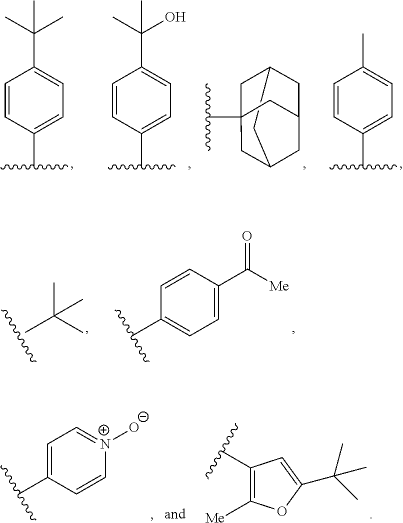

101. The compound of any one of claims 1-6, 9, 29-51, 64-66, 69-70, 86-98, or 100, wherein B is independently selected from the group consisting of hydrogen, methyl,

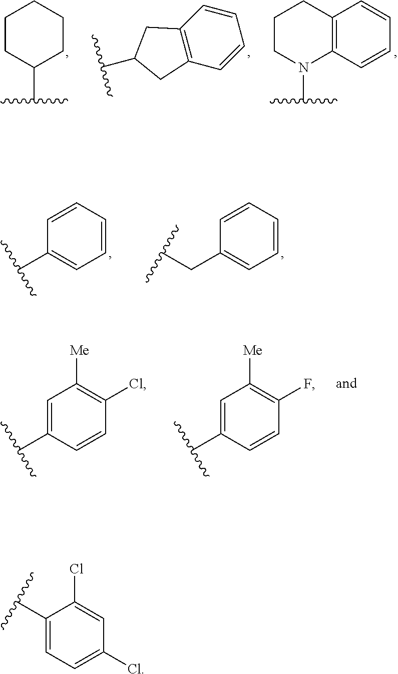

102. The compound of any one of claims 3-5, 29-76, 86-98, or 100-101, wherein n is 0.

103. The compound of any one of claims 3-5, 29-76, 86-98, or 100-101, wherein n is 1.

104. The compound of any one of claims 3-5, 29-76, 86-98, or 100-101, wherein n is 2.

105. The compound of any one of claims 3-5, 29-76, 86-98, or 100-101, wherein n is 3.

106. The compound of any one of claims 3-4, 29-76, 86-98, or 100-101, wherein T is hydrogen.

107. The compound of any one of claims 3-4, 29-76, 86-98, or 100-101, wherein T is halogen.

108. The compound of any one of claims 3-4, 29-76, 86-98, or 100-101, 107, wherein T is F.

109. The compound of any one of claims 3-4, 29-76, 86-98, or 100-101, wherein T is optionally substituted C₁₋₆ alkyl.

110. The compound of any one of claims 3-4, 29-76, 86-98, 100-101, or 109, wherein T is unsubstituted C₁₋₆ alkyl.

111. The compound of any one of claims 3-4, 29-76, 86-98, 100-101, or 109-110, wherein T is methyl.

112. The compound of any one of claims 3-4, 29-76, 86-98, or 100-101, wherein T is —OR^{T}.

113. The compound of any one of claims 3-4, 29-76, 86-98, 100-101, or 112, wherein T is —OH.

114. The compound of any one of claims 1-5, 9-52, 54-58, 61-64, 66-82, or 85-113, wherein R₁ is hydrogen.

115. The compound of any one of claims 1-5, 9-52, 54-58, 61-64, 66-82, or 85-113, wherein R₁ is halogen.

116. The compound of any one of claims 1-5, 9-52, 54-58, 61-64, 66-82, or 85-113, wherein R₁ is F.

117. The compound of any one of claims 1-5, 9-52, 54-58, 61-64, 66-82, or 85-113, wherein R₁ is optionally substituted alkyl.

118. The compound of any one of claims 1-5, 9-52, 54-58, 61-64, 66-82, or 85-113, wherein R₁ is benzyl.

119. The compound of any one of claims 1-5, 9-52, 54-58, 61-64, 66-82, 85-113, or 117, wherein R₁ is optionally substituted methyl.

120. The compound of any one of claims 1-5, 9-52, 54-58, 61-64, 66-82, 85-113, 117, or 119, wherein R₁ is of the formula: —CH₂(R_{X}), wherein R_{X} is carbocyclyl, aryl, or heteroaryl.

121. The compound of claim 120 wherein R₁ is of the formula:

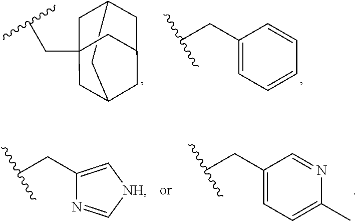

122. The compound of any one of claims 1-5, 7, 9-52, 55-58, 61-64, 66-82, or 85-121, wherein R^{N1} is H.

123. The compound of any one of claims 1-122 having one of the following structures:

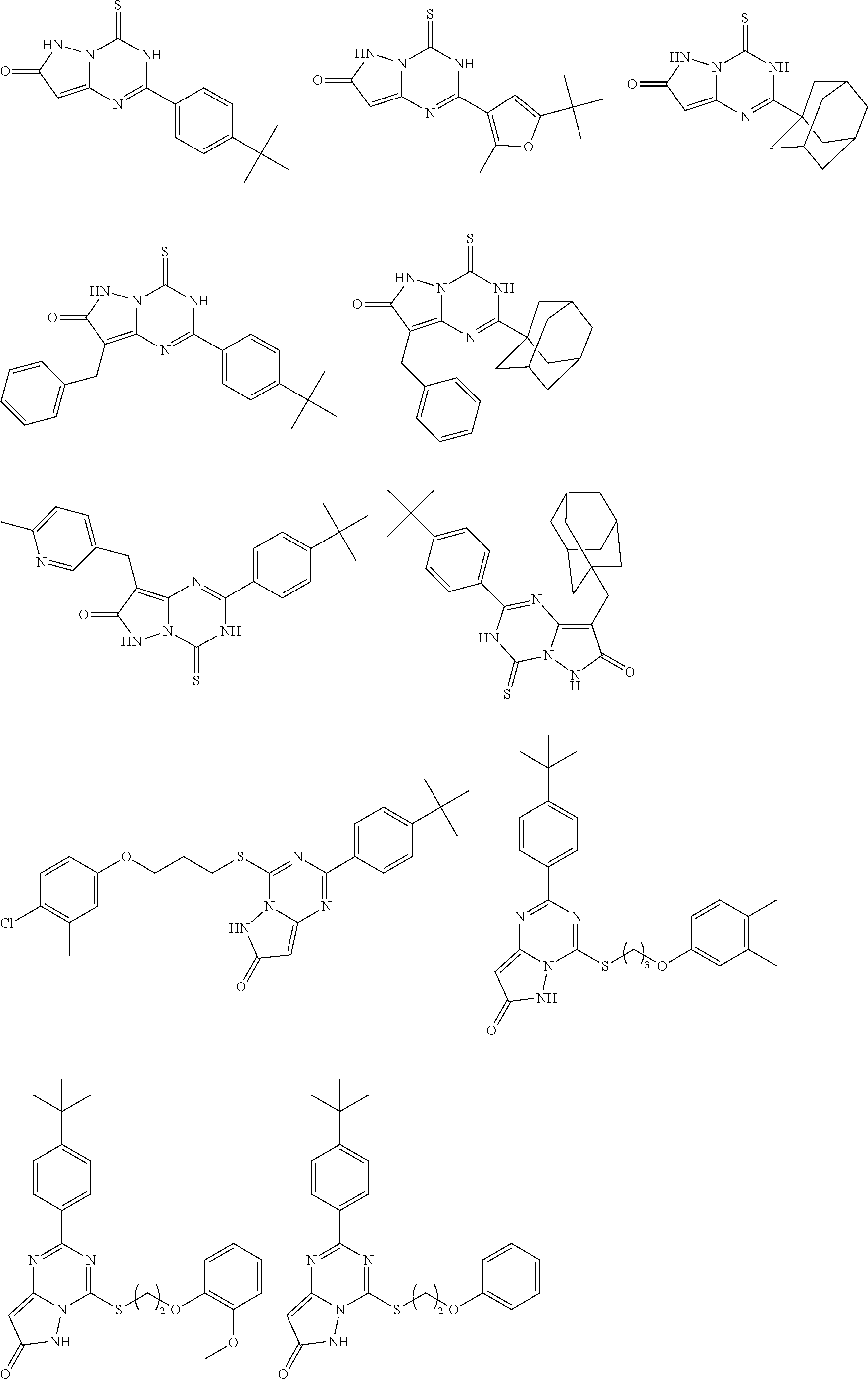

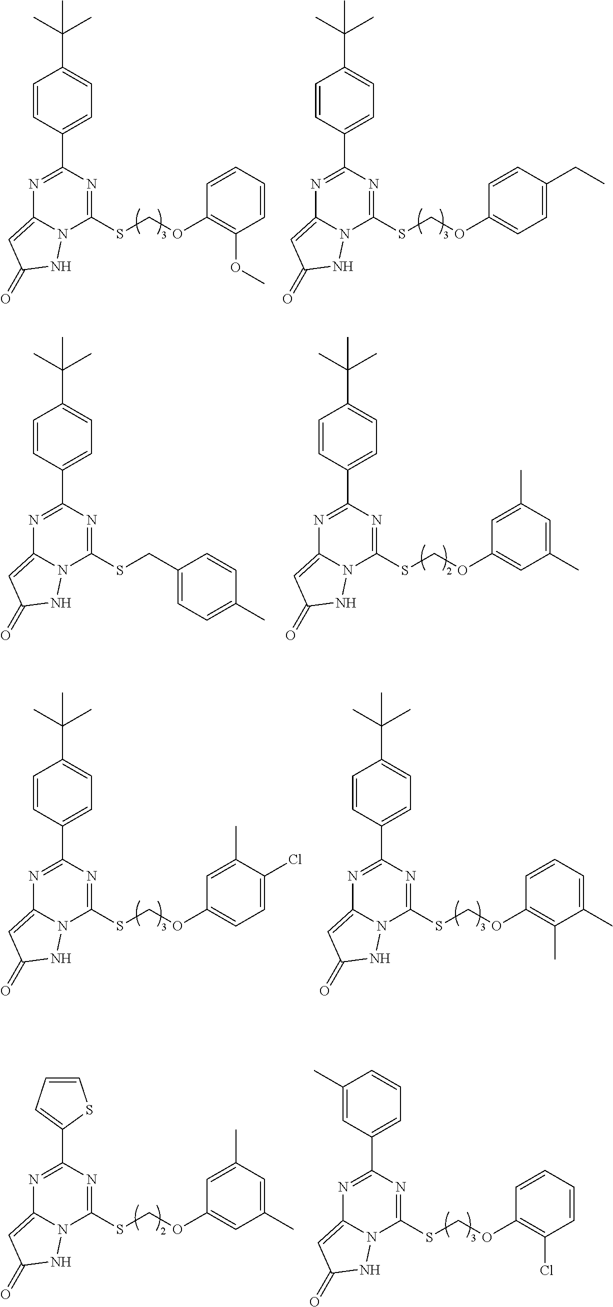

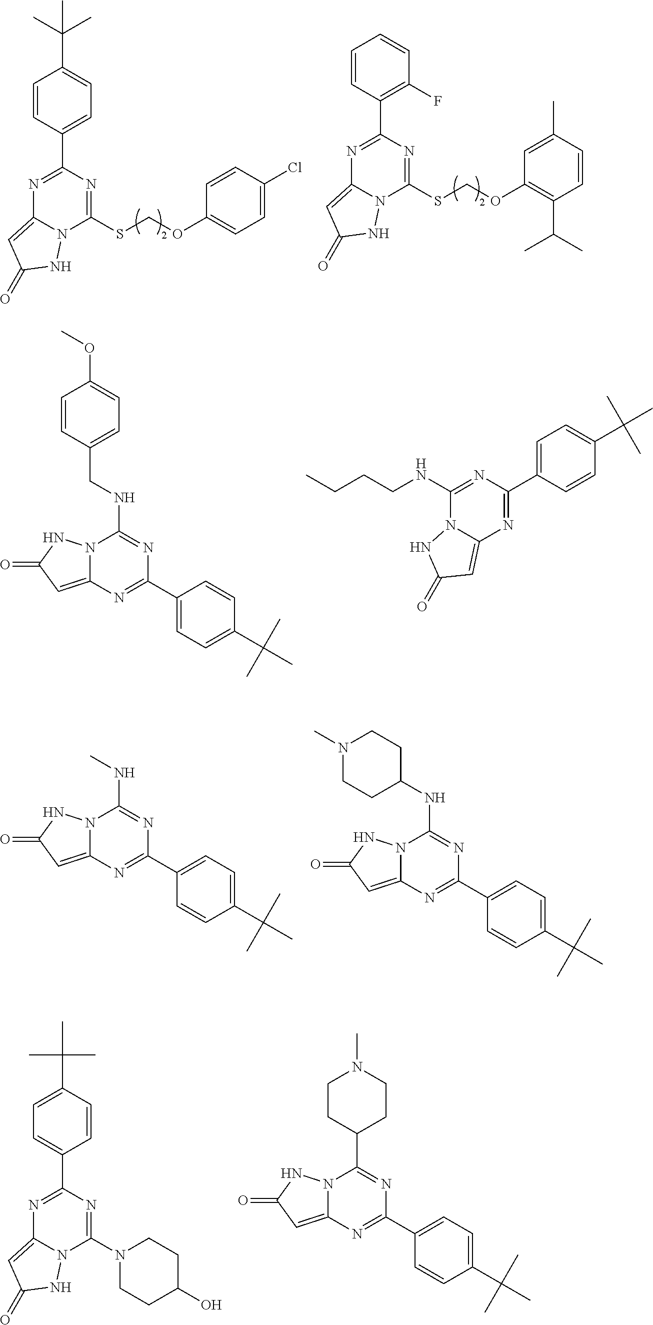

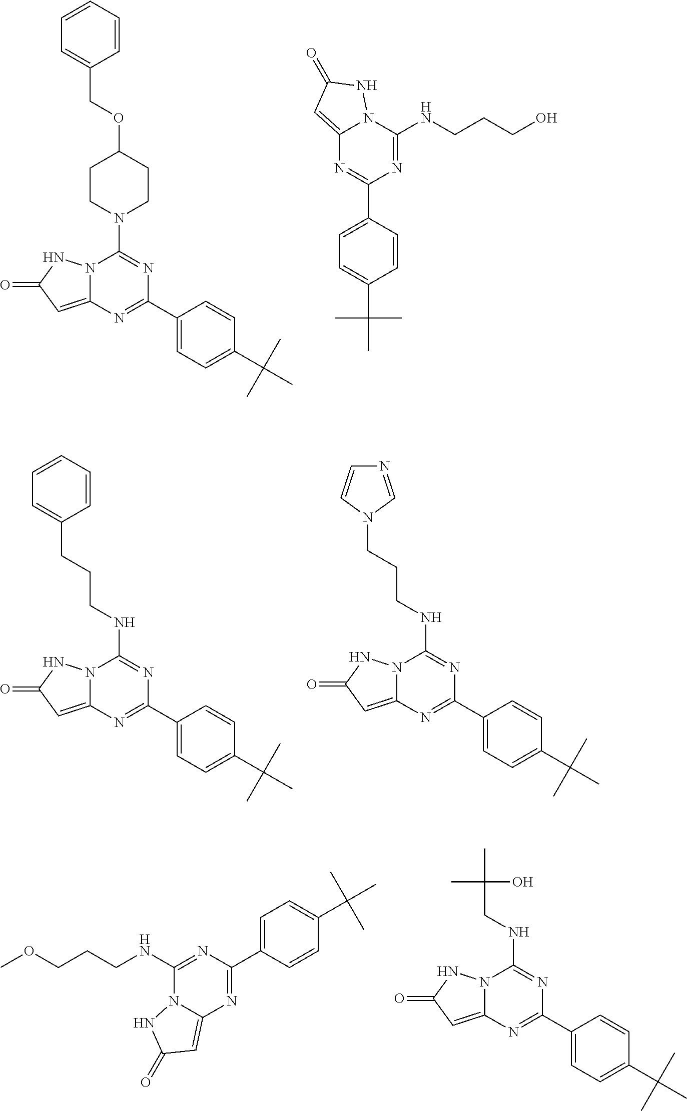 [SMILES: CC(C)(C)c1ccc(-c2nc(N3CCC(OCc4ccccc4)CC3)n3[nH]c(=O)cc3n2)cc1 ; CC(C)(C)c1ccc(-c2nc(NCCCO)n3[nH]c(=O)cc3n2)cc1 ; CC(C)(C)c1ccc(-c2nc(NCCCc3ccccc3)n3[nH]c(=O)cc3n2)cc1 ; CC(C)(C)c1ccc(-c2nc(NCCCn3ccnc3)n3[nH]c(=O)cc3n2)cc1 ; COCCCNc1nc(-c2ccc(C(C)(C)C)cc2)nc2cc(=O)[nH]n12 ; CC(C)(O)CNc1nc(-c2ccc(C(C)(C)C)cc2)nc2cc(=O)[nH]n12]

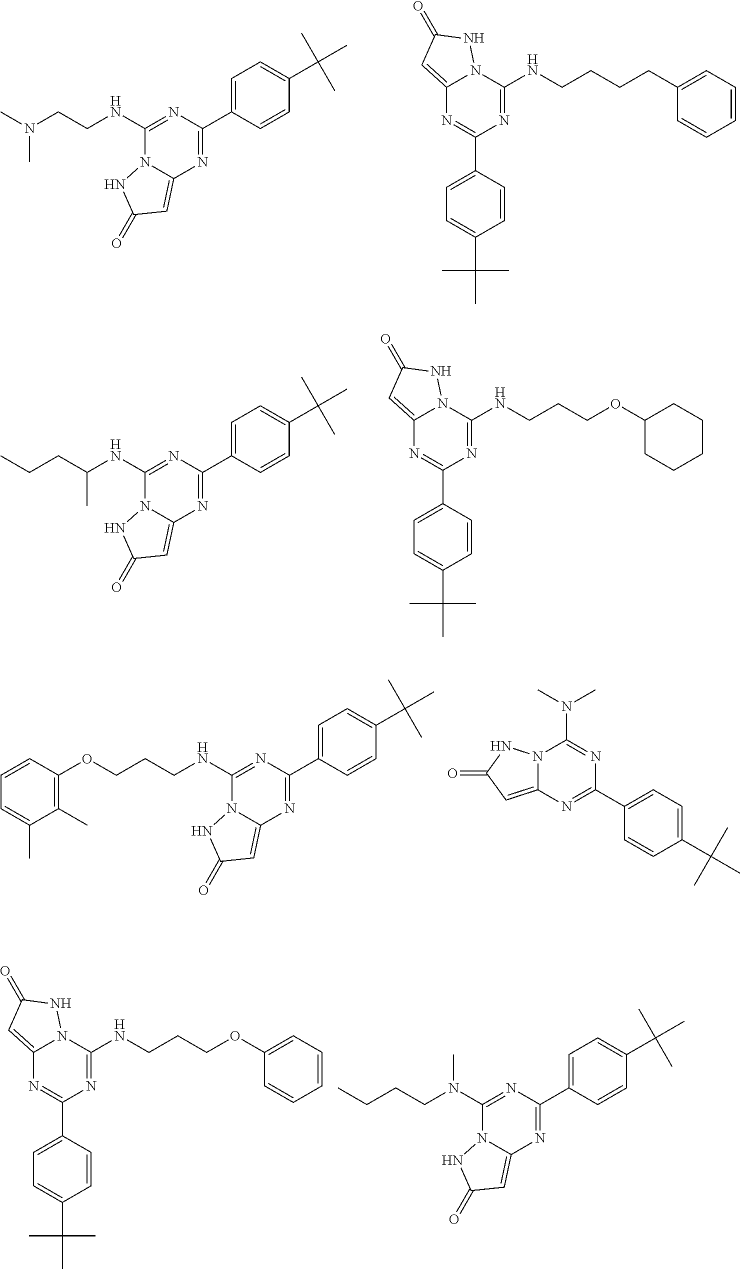

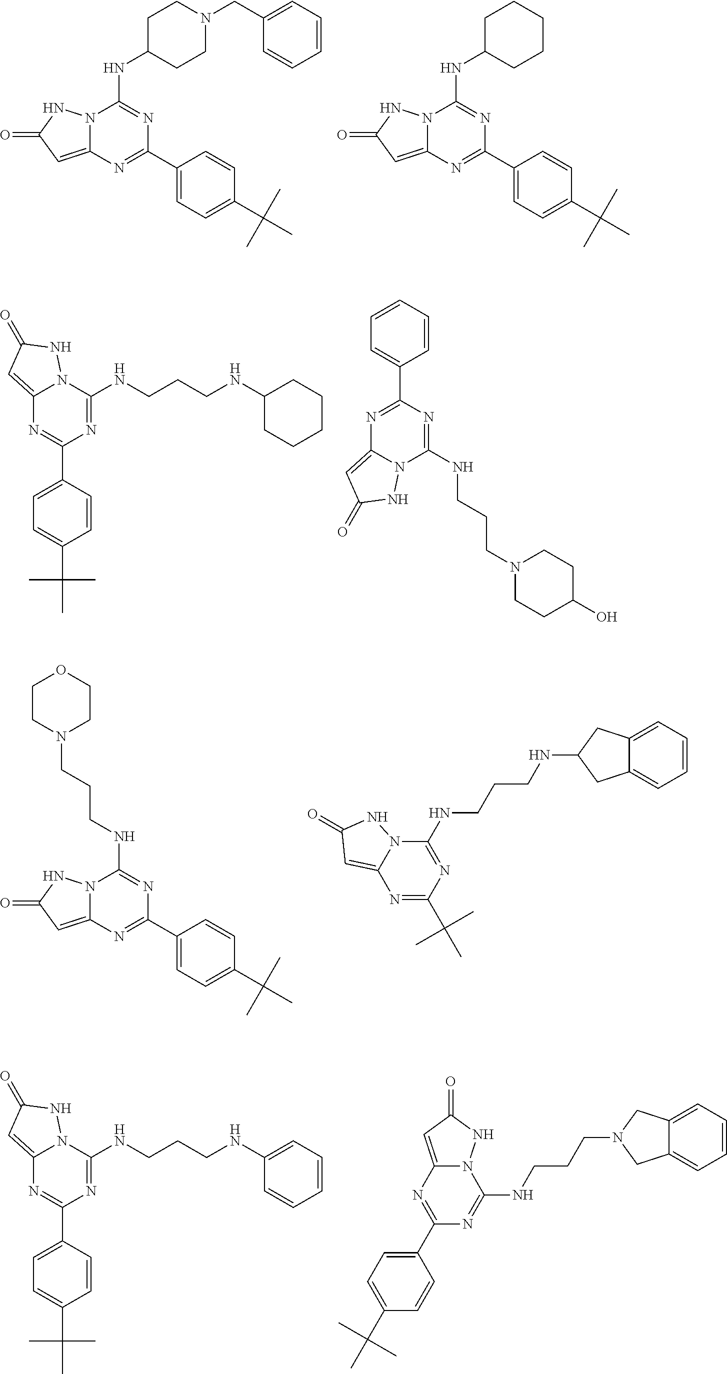

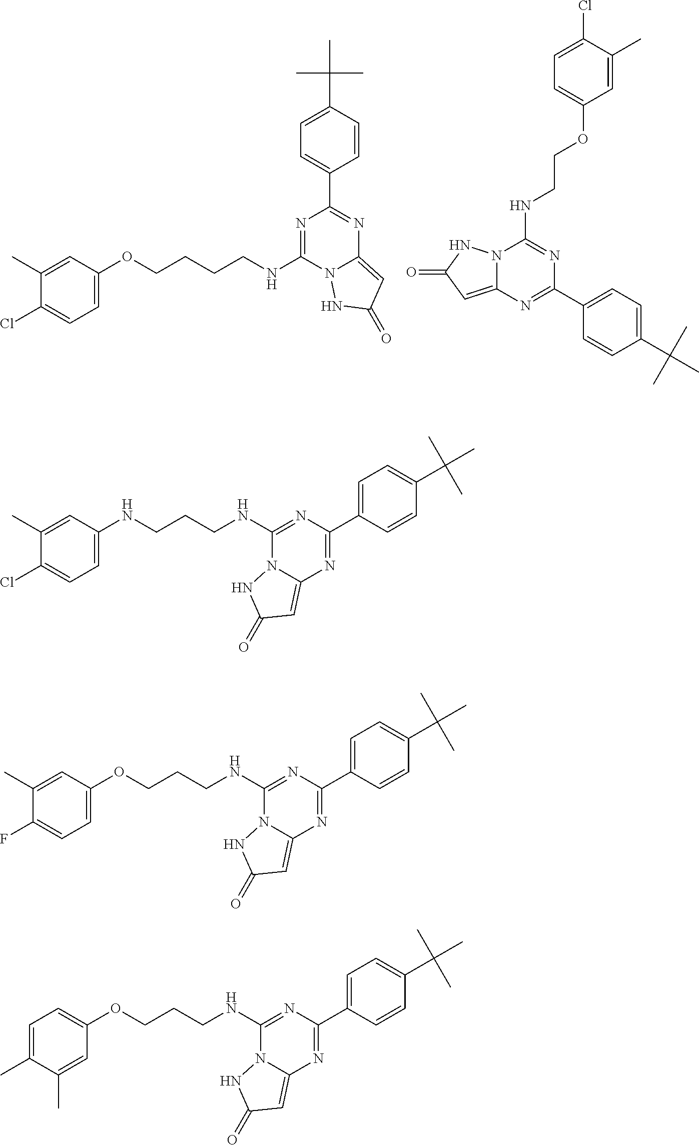

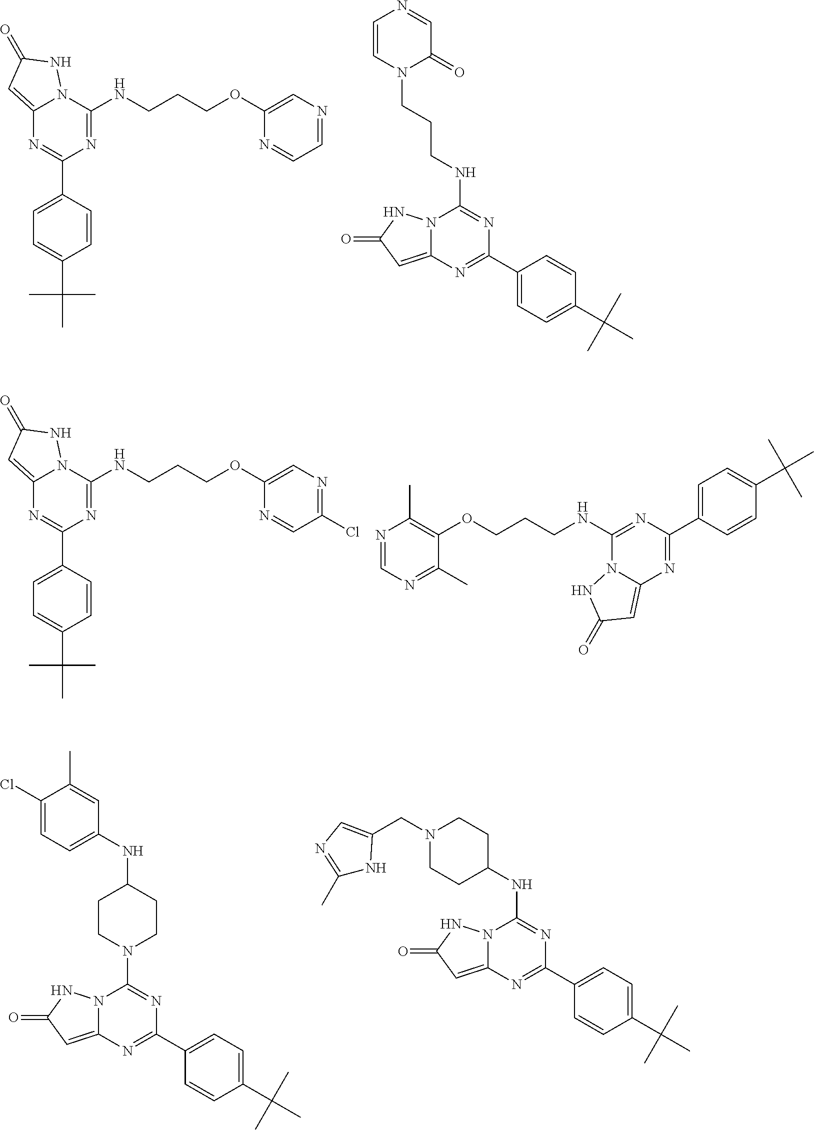

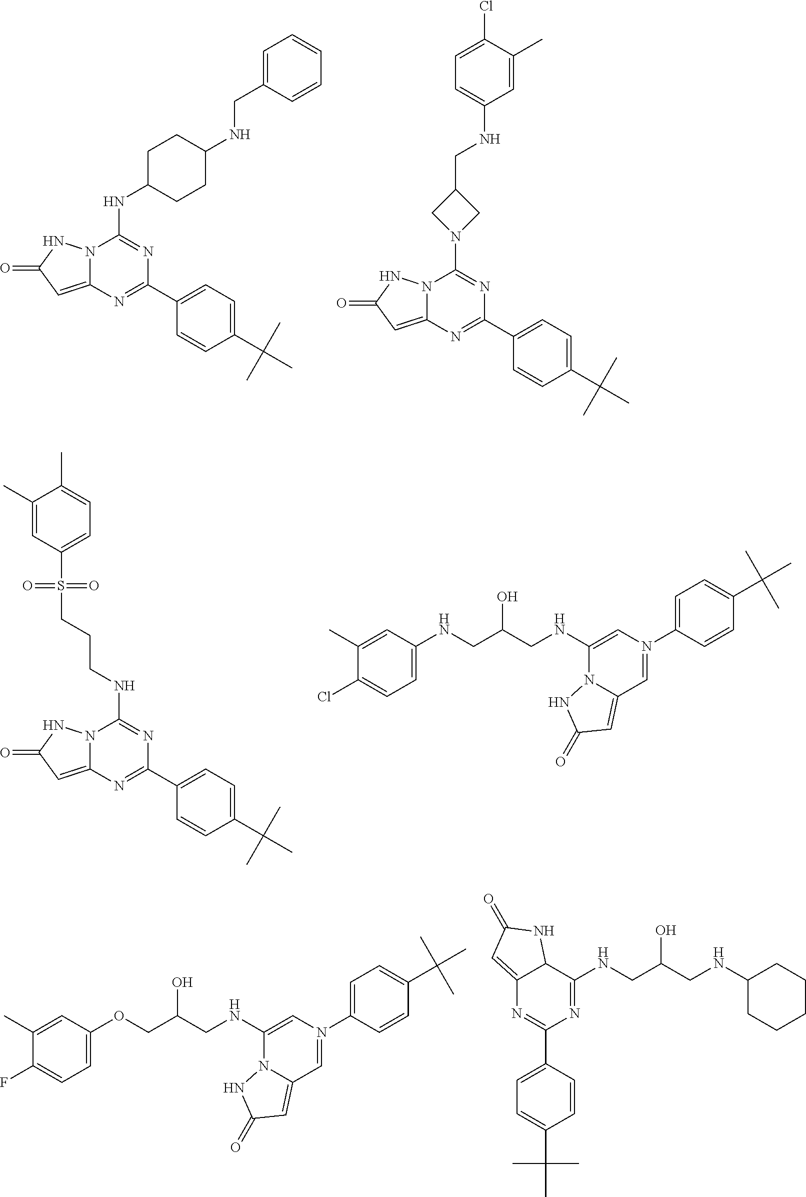

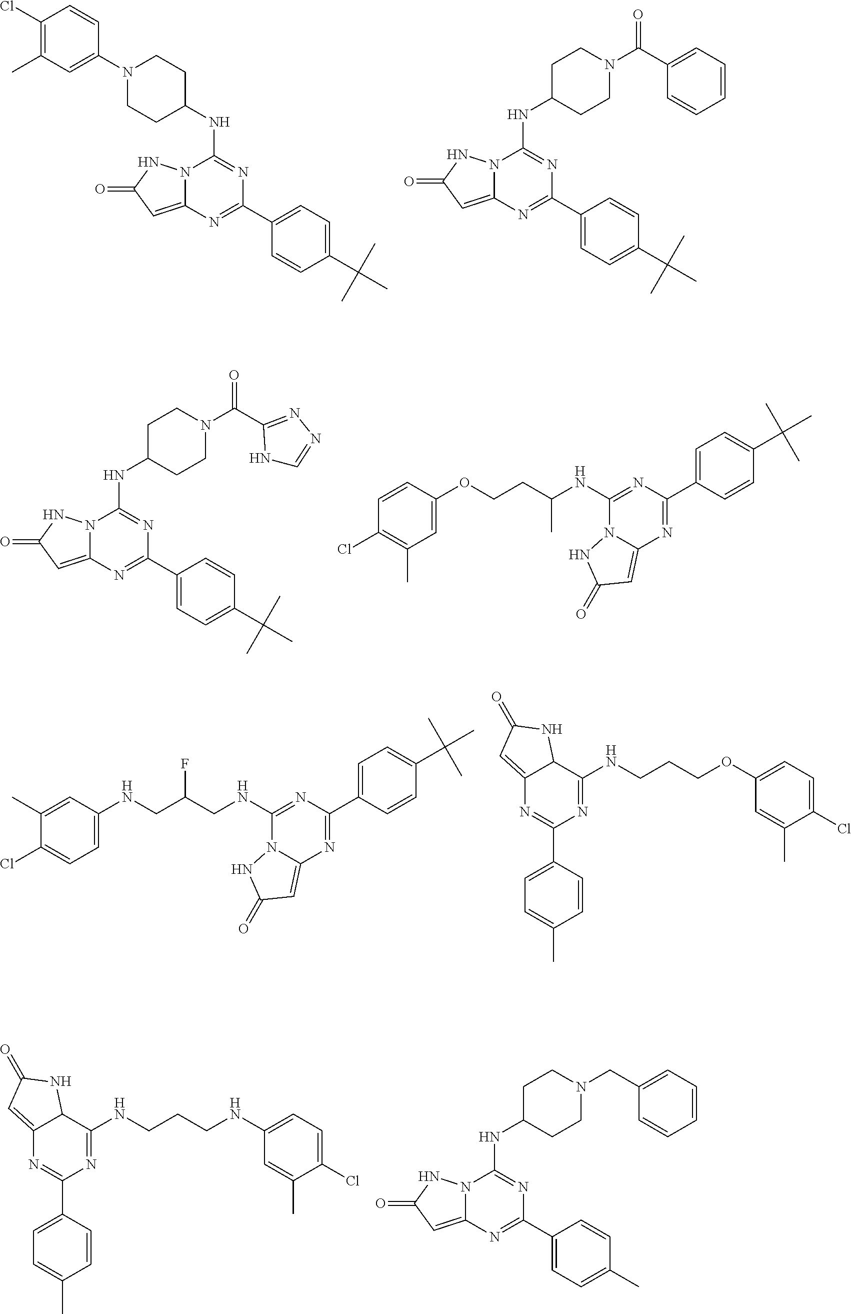

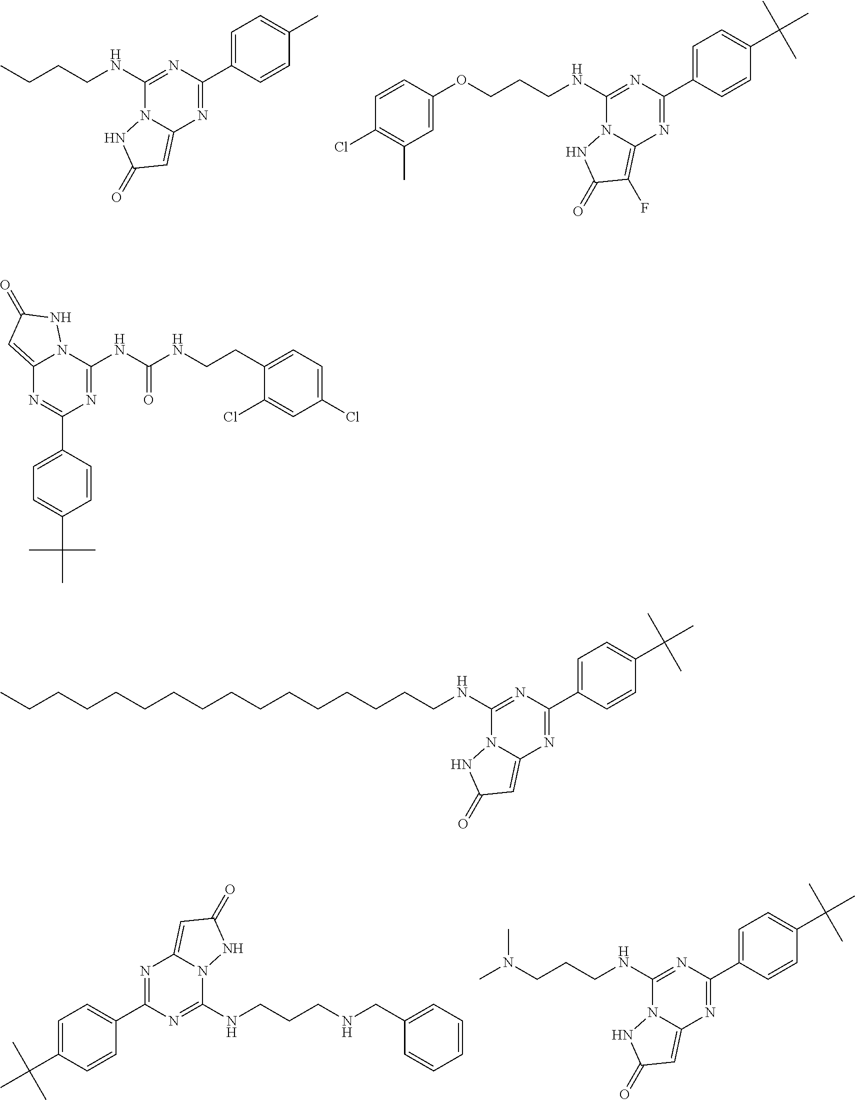

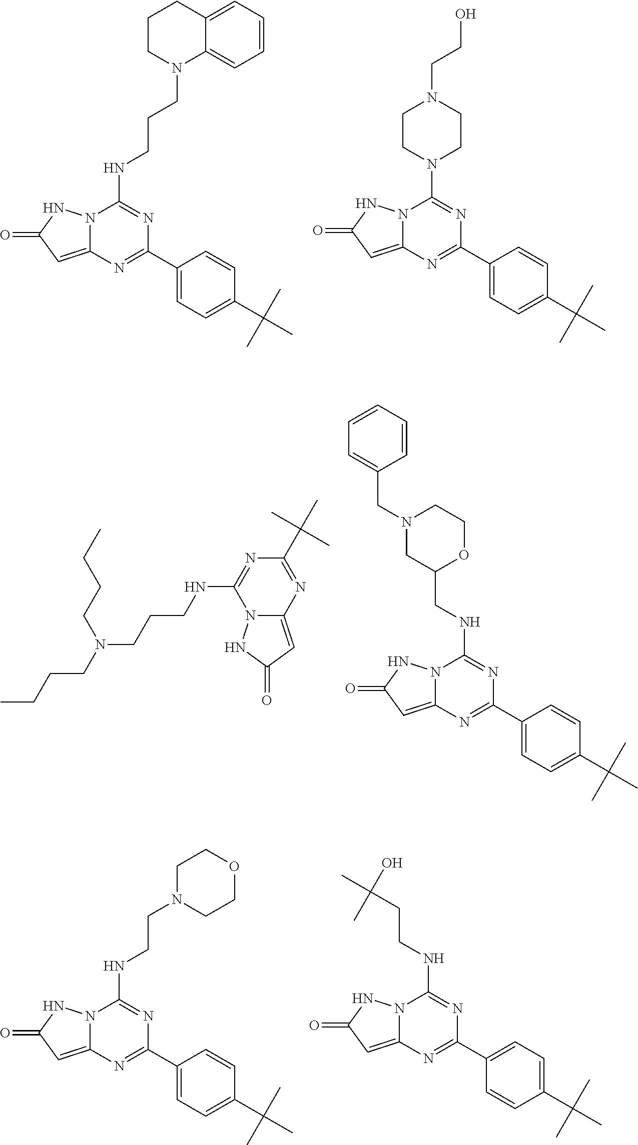 [SMILES: CC(C)(C)c1ccc(-c2nc(NCCCN3CCCc4ccccc43)n3[nH]c(=O)cc3n2)cc1 ; CC(C)(C)c1ccc(-c2nc(N3CCN(CCO)CC3)n3[nH]c(=O)cc3n2)cc1 ; CCCCN(CCCC)CCCNc1nc(C(C)(C)C)nc2cc(=O)[nH]n12 ; CC(C)(C)c1ccc(-c2nc(NCC3CN(Cc4ccccc4)CCO3)n3[nH]c(=O)cc3n2)cc1 ; CC(C)(C)c1ccc(-c2nc(NCCN3CCOCC3)n3[nH]c(=O)cc3n2)cc1 ; CC(C)(O)CCNc1nc(-c2ccc(C(C)(C)C)cc2)nc2cc(=O)[nH]n12]

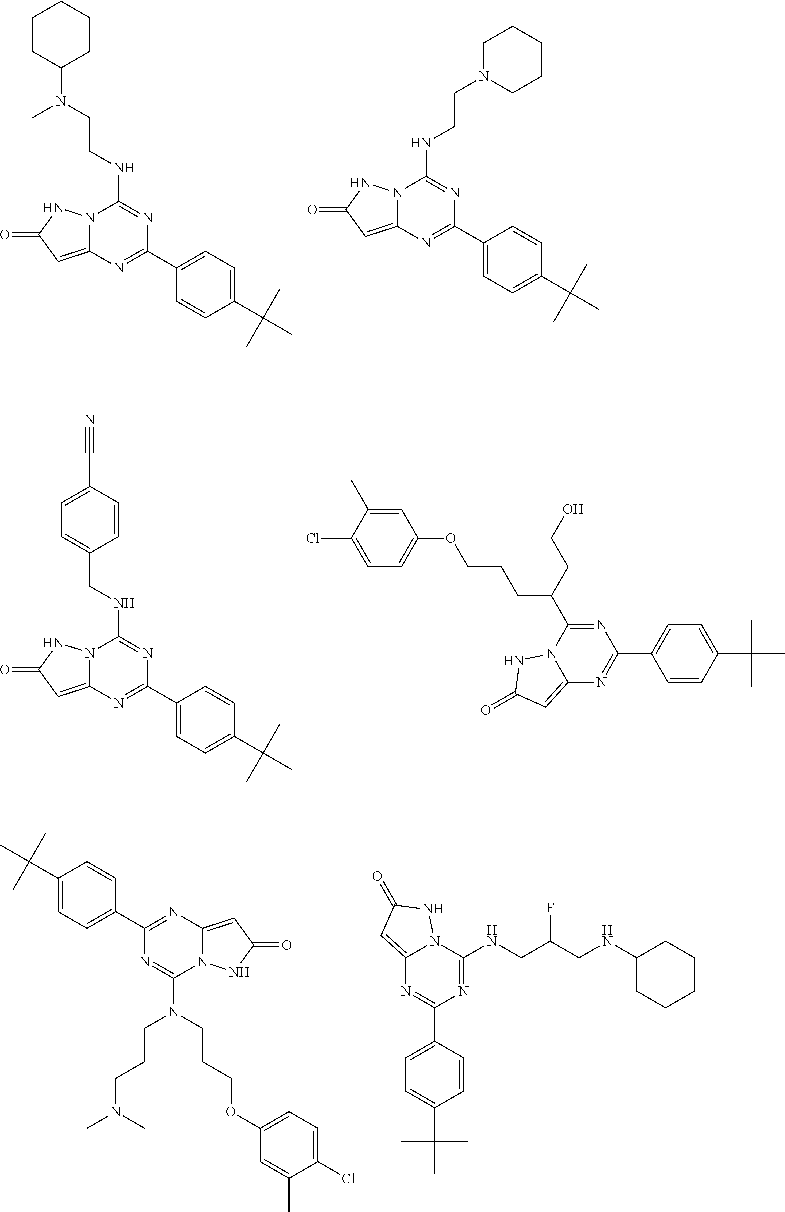 [SMILES: CN(CCNc1nc(-c2ccc(C(C)(C)C)cc2)nc2cc(=O)[nH]n12)C1CCCCC1 ; CC(C)(C)c1ccc(-c2nc(NCCN3CCCCC3)n3[nH]c(=O)cc3n2)cc1 ; CC(C)(C)c1ccc(-c2nc(NCc3ccc(C#N)cc3)n3[nH]c(=O)cc3n2)cc1 ; Cc1cc(OCCCC(CCO)c2nc(-c3ccc(C(C)(C)C)cc3)nc3cc(=O)[nH]n23)ccc1Cl ; Cc1cc(OCCCN(CCCN(C)C)c2nc(-c3ccc(C(C)(C)C)cc3)nc3cc(=O)[nH]n23)ccc1Cl ; CC(C)(C)c1ccc(-c2nc(NCC(F)CNC3CCCCC3)n3[nH]c(=O)cc3n2)cc1]

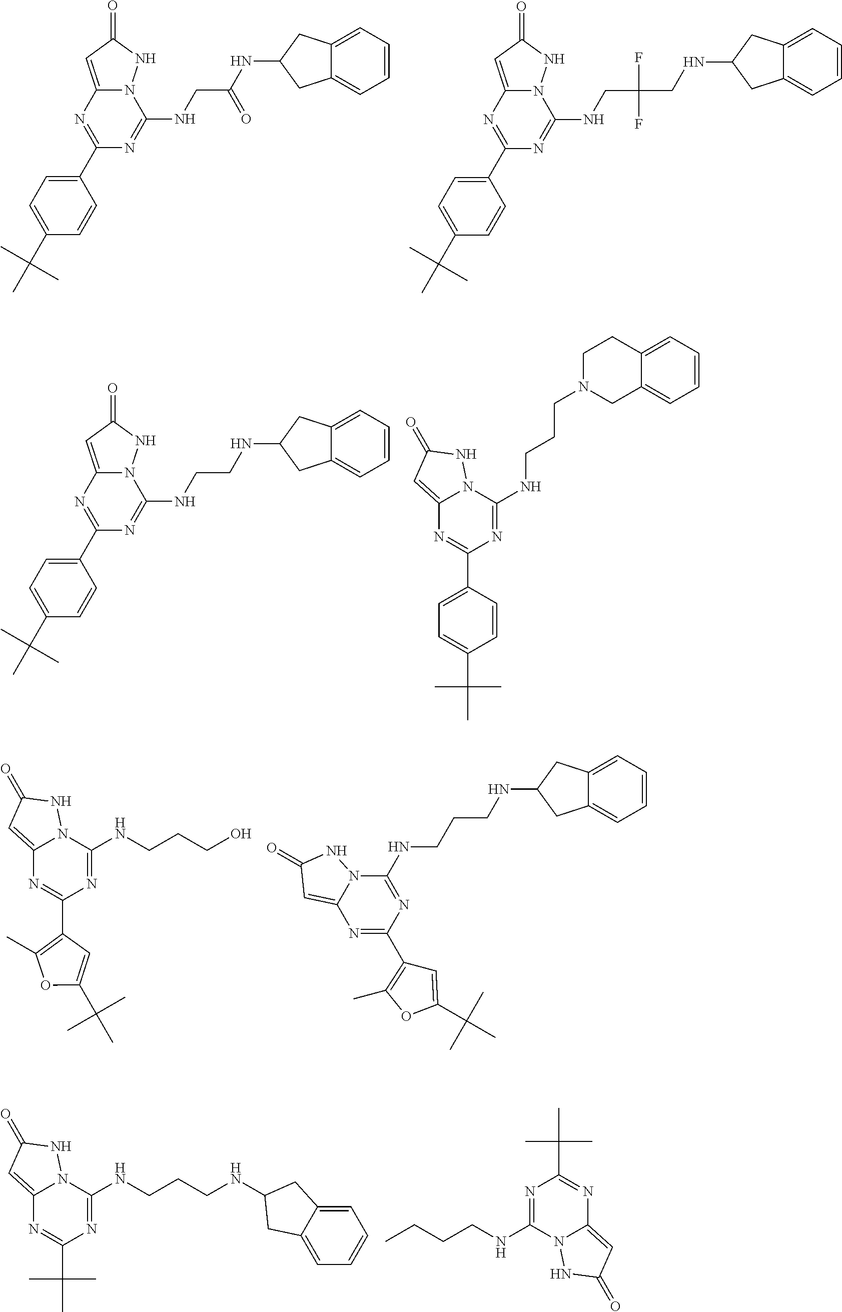

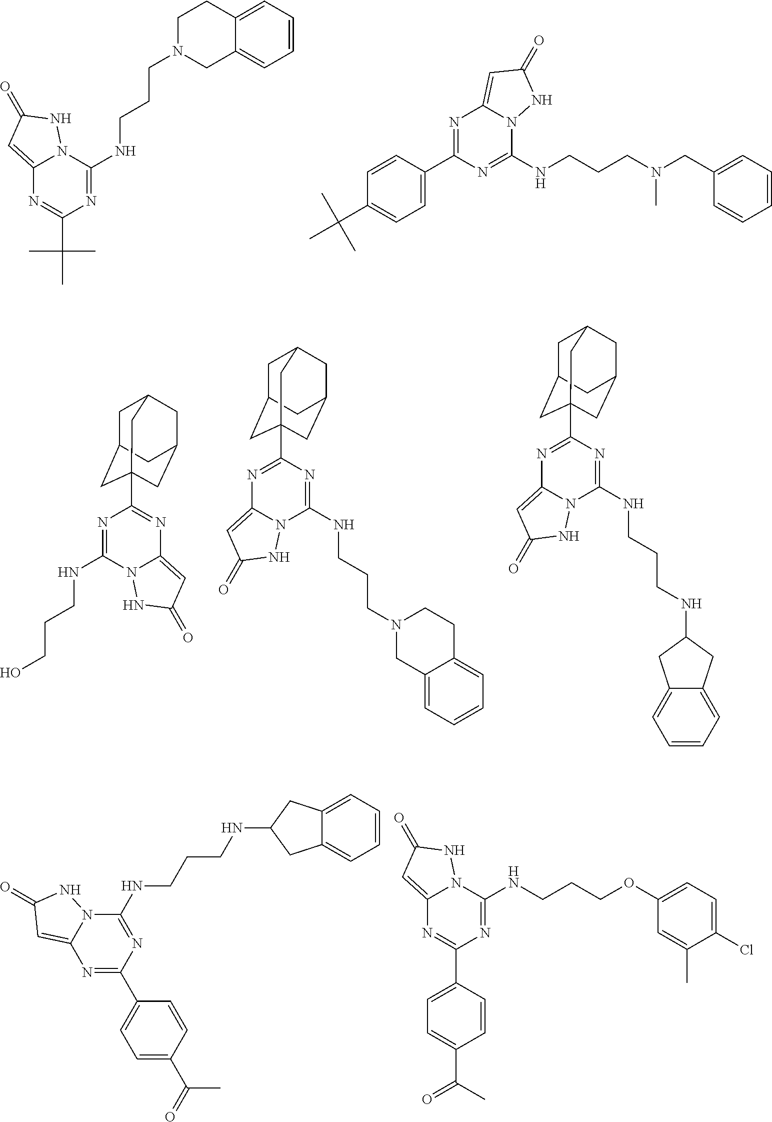

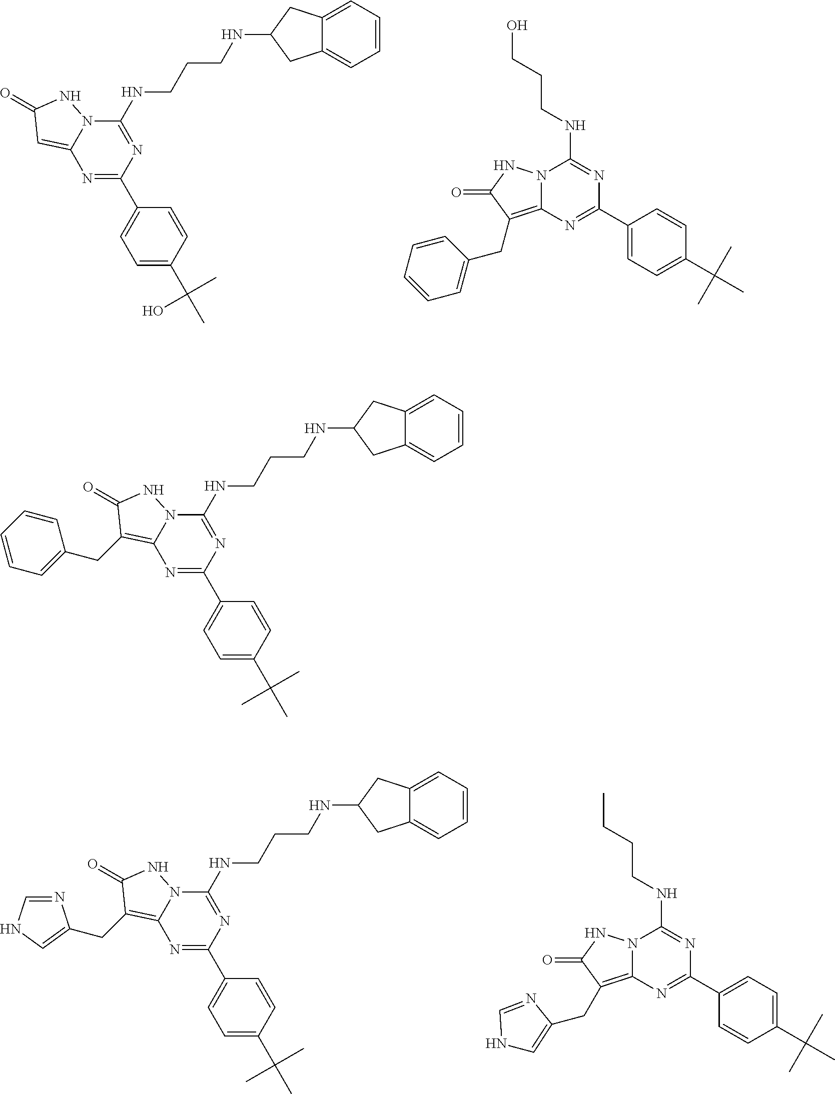 [SMILES: CC(C)(O)c1ccc(-c2nc(NCCCNC3Cc4ccccc4C3)n3[nH]c(=O)cc3n2)cc1 ; CC(C)(C)c1ccc(-c2nc(NCCCO)n3[nH]c(=O)c(Cc4ccccc4)c3n2)cc1 ; CC(C)(C)c1ccc(-c2nc(NCCCNC3Cc4ccccc4C3)n3[nH]c(=O)c(Cc4ccccc4)c3n2)cc1 ; CC(C)(C)c1ccc(-c2nc(NCCCNC3Cc4ccccc4C3)n3[nH]c(=O)c(Cc4c[nH]cn4)c3n2)cc1 ; CCCCNc1nc(-c2ccc(C(C)(C)C)cc2)nc2c(Cc3c[nH]cn3)c(=O)[nH]n12]

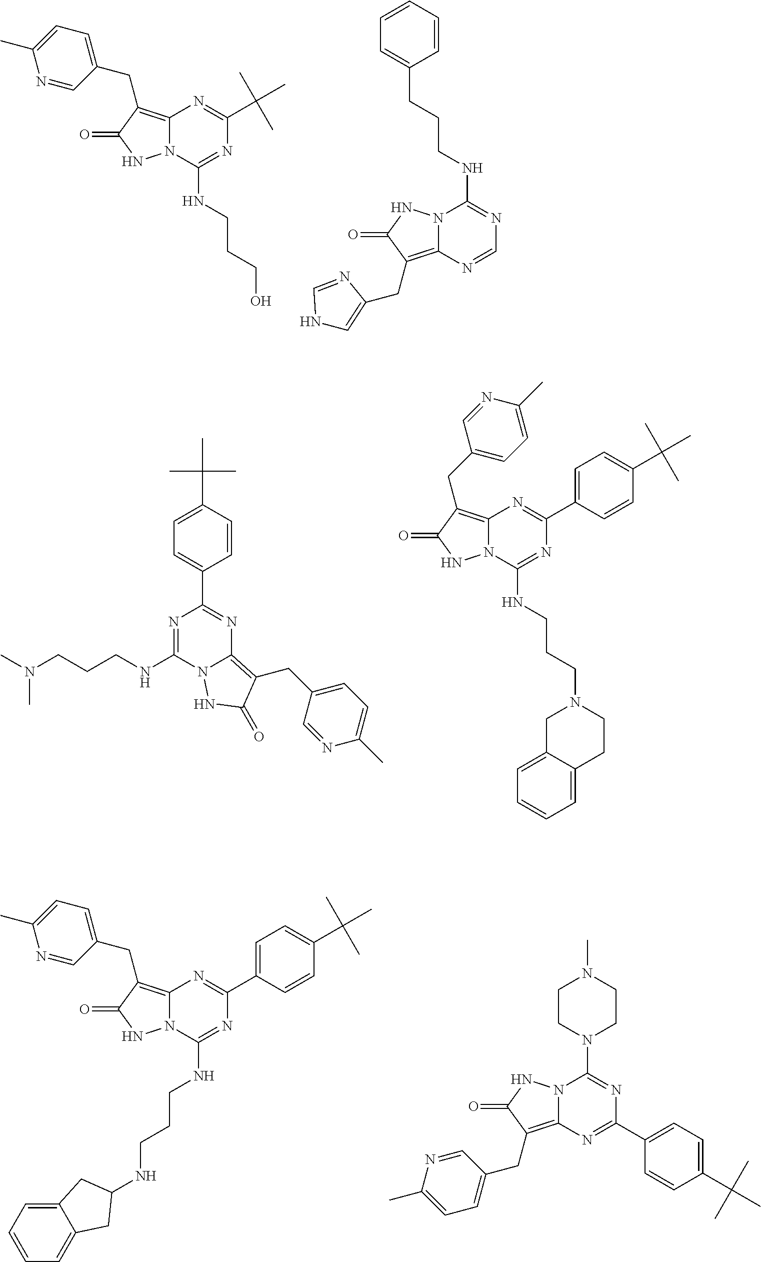

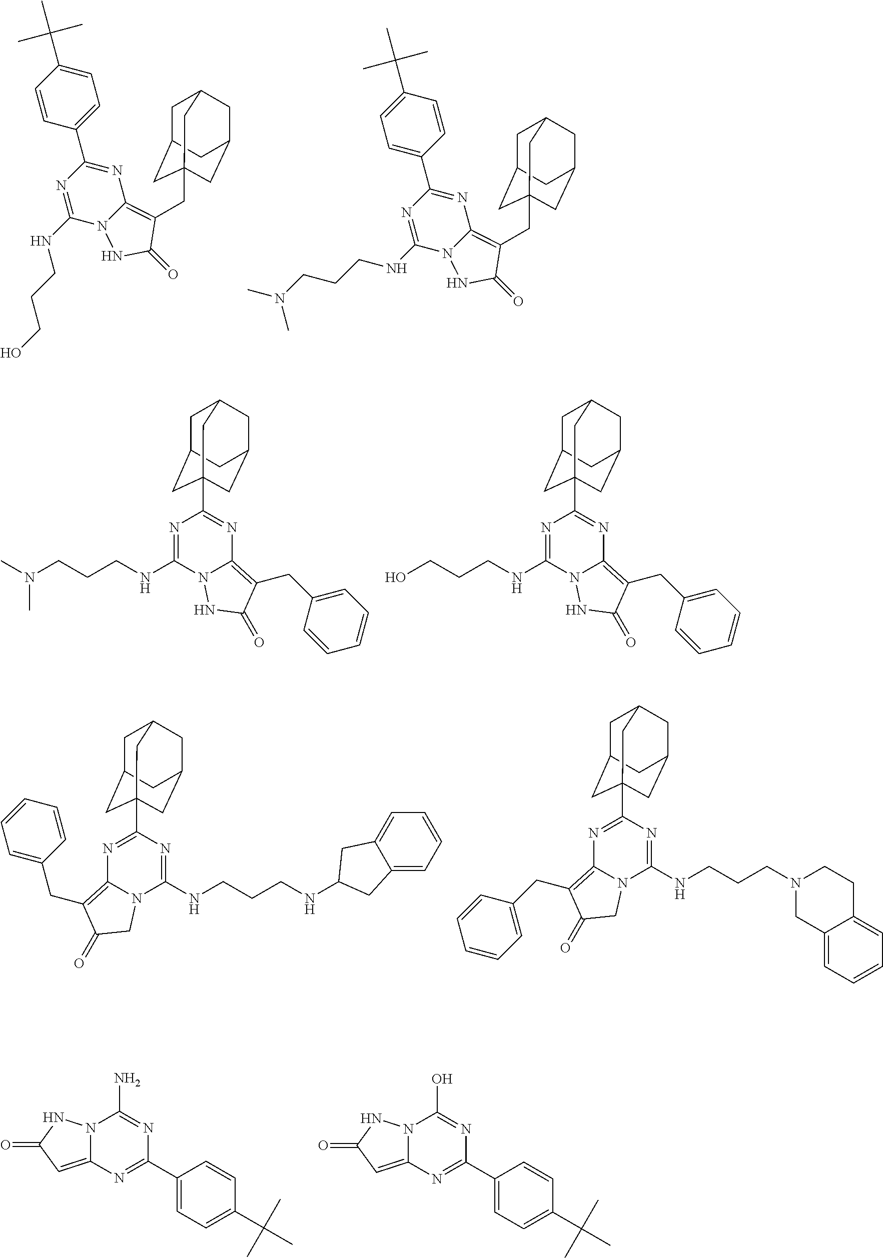

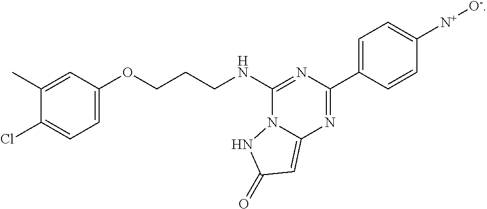

124. A pharmaceutical composition comprising a compound of any one of claims 1-123, or a pharmaceutically acceptable salt, solvate, hydrate, polymorph, co-crystal, tautomer, stereoisomer, isotopically labeled derivative, or prodrug thereof, and optionally a pharmaceutically acceptable carrier.

125. A pharmaceutical composition for use in treating and/or preventing a bacterial infection comprising a compound of any one of claims 1-123, or a pharmaceutically acceptable salt, solvate, hydrate, polymorph, co-crystal, tautomer, stereoisomer, isotopically labeled derivative, or prodrug thereof, and optionally a pharmaceutically acceptable carrier.

126. A kit comprising a compound of any one of claims 1-123, or a pharmaceutically acceptable salt, solvate, hydrate, polymorph, co-crystal, tautomer, stereoisomer, isotopically labeled derivative, or prodrug thereof, and instructions for use thereof.

127. A method of treating and/or preventing a bacterial infection comprising administering to a subject in need thereof a therapeutically effective amount of a compound according to any one of claims 1-123, or a pharmaceutically acceptable salt, solvate, hydrate, polymorph, co-crystal, tautomer, stereoisomer, isotopically labeled derivative, or prodrug thereof, or a pharmaceutical composition of claims 124 or 125.

128. A method of treating and/or preventing a bacterial infection comprising administering to a subject in need thereof a therapeutically effective amount of a compound according to any one of claims 1 or 2, or a compound of Formula (IA), 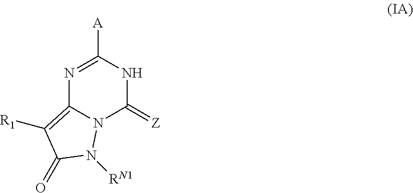
or a pharmaceutically acceptable salt, solvate, hydrate, polymorph, co-crystal, tautomer, stereoisomer, isotopically labeled derivative, or prodrug thereof,
wherein
A is independently optionally substituted C₂₋₆ alkyl, optionally substituted aryl, optionally substituted C₄₋₁₀ carbocyclyl, optionally substituted 5-membered heteroaryl, or optionally substituted 6-membered heteroaryl;
Z is S or O;
R₁ is hydrogen, halogen, or optionally substituted C₁₋₆ alkyl;
R^{N1} is hydrogen, optionally substituted C₁₋₆ alkyl, or a nitrogen protecting group; and R^{NX} is hydrogen, optionally substituted C₁₋₆ alkyl, or a nitrogen protecting group; or a pharmaceutical composition of claims 124 or 125; provided that the compound is not of the formula:

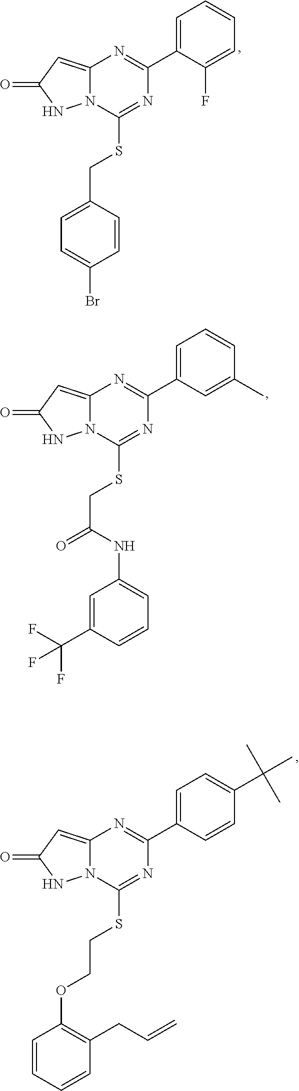 [SMILES: O=c1cc2nc(-c3ccccc3F)nc(SCc3ccc(Br)cc3)n2[nH]1 ; Cc1cccc(-c2nc(SCC(=O)Nc3cccc(C(F)(F)F)c3)n3[nH]c(=O)cc3n2)c1 ; C=CCc1ccccc1OCCSc1nc(-c2ccc(C(C)(C)C)cc2)nc2cc(=O)[nH]n12]

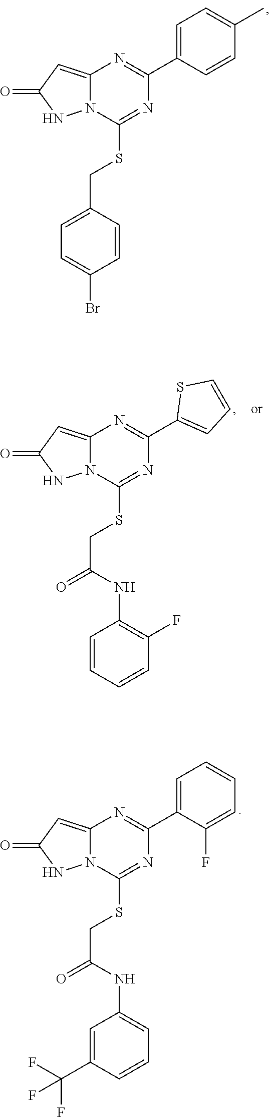 [SMILES: Cc1ccc(-c2nc(SCc3ccc(Br)cc3)n3[nH]c(=O)cc3n2)cc1 ; O=C(CSc1nc(-c2cccs2)nc2cc(=O)[nH]n12)Nc1ccccc1F ; O=C(CSc1nc(-c2ccccc2F)nc2cc(=O)[nH]n12)Nc1cccc(C(F)(F)F)c1]

129. The method of claim 128, comprising administering to a subject in need thereof a therapeutically effective amount of a compound according to any one of claim 1 or a compound of Formula (IA).

130. The method of any one of claims 127-129, wherein the bacterial infection is an infection with a Gram-negative bacterium.

131. The method of any one claims 127-129, wherein the bacterial infection is an infection with a Gram-positive bacterium.

132. The method of claim 130, wherein the Gram-negative bacterium is selected from the group consisting of Escherichia coli, Citrobacter spp, Enterobacter spp, Klebsiella spp, Proteus spp, Serratia spp, Shigella spp, Salmonella spp, Morganella morganii, Providencia spp, Edwardsiella spp. Erwinia spp, Hafnia spp, Yersinia spp, Acinetobacter spp, Vibrio spp, Aeromonas spp, Pseudomonas spp, Haemophilus spp, Pasteurella spp, Campylobacter spp, Helicobacter spp, Branhamella catarrhalis, Moraxella spp, Neisseria spp, Veillonella parvula, Fusobacterium spp, Bacteroides spp, Actinobacillus actinomycetemcomitans, Aggregatibacter actinomycetemcomitans, Agrobacterium spp, Porphyromonas spp, Prevotella spp, Ruminobacter spp, Roseburia spp, Caulobacter crescentus, Francisella spp, Borrelia spp, Treponema pallidum, Brucella spp, and Rickettsia.

133. The method of claim 131, wherein the Gram-positive bacterium is selected from the group consisting of Staphylococcus spp, Streptococcus spp, Micrococcus spp, Peptococcus spp, Peptostreptococcus spp, Enterococcus spp, Bacillus spp, Clostridium spp, Lactobacillus spp, Listeria spp, Erysipelothrix spp, Propionibacterium spp, Eubacterium spp, Corynebacterium spp, Capnocytophaga spp, Bifidobacterium spp, and Gardnerella spp.

134. The method of any one of claims 127-129, wherein the bacterium is resistant to methicillin.

135. The method of any one of claims 127-129, wherein the bacterium is resistant to vancomycin.

136. The method of any one of claims 127-135, wherein the compound is administered parenterally, intramuscularly, intravenously, subcutaneously, orally, topically or intranasally.

137. A method for inhibiting bacterial cell growth comprising contacting bacteria with a compound according to any one of claims 1-123, or a pharmaceutically acceptable salt, solvate, hydrate, polymorph, co-crystal, tautomer, stereoisomer, isotopically labeled derivative, or prodrug thereof, or a pharmaceutical composition of claims 124 or 125.

138. A method for inducing bacterial hypersusceptibility comprising contacting a bacterium with a compound according to any one of claims 1-123, or a pharmaceutically acceptable salt, solvate, hydrate, polymorph, co-crystal, tautomer, stereoisomer, isotopically labeled derivative, or prodrug thereof, or a pharmaceutical composition of claims 124 or 125.

139. The method of claims 137 or 138, wherein the bacteria is contacted with the compound in vitro.

140. The method of claims 137 or 138, wherein the bacteria is contacted with the compound in vivo.

141. The method of any one claims 127-140, wherein the compound is administered with another antibiotic.

142. The method of any one of claims 127-141, wherein the compound is of one of the following structures:

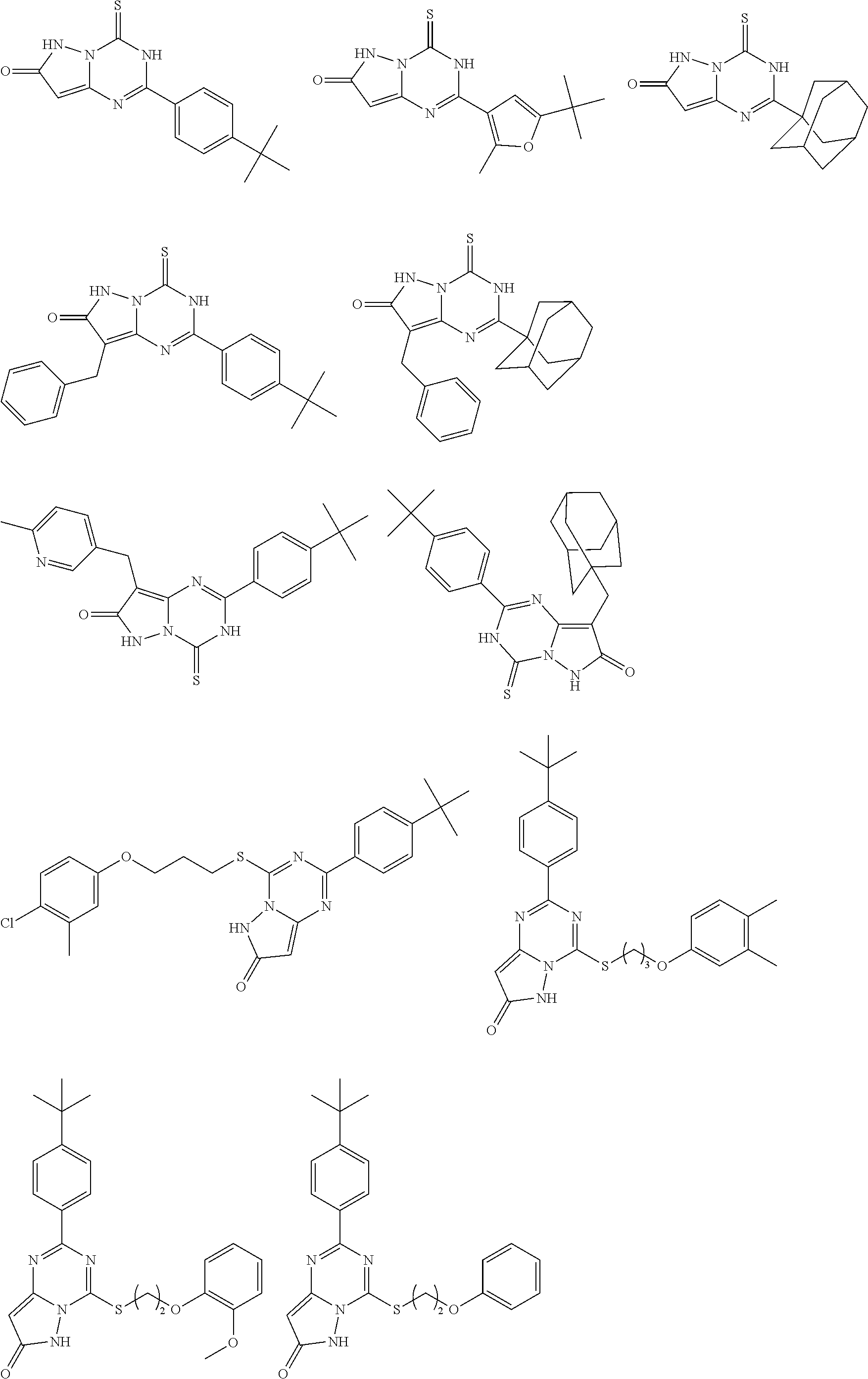

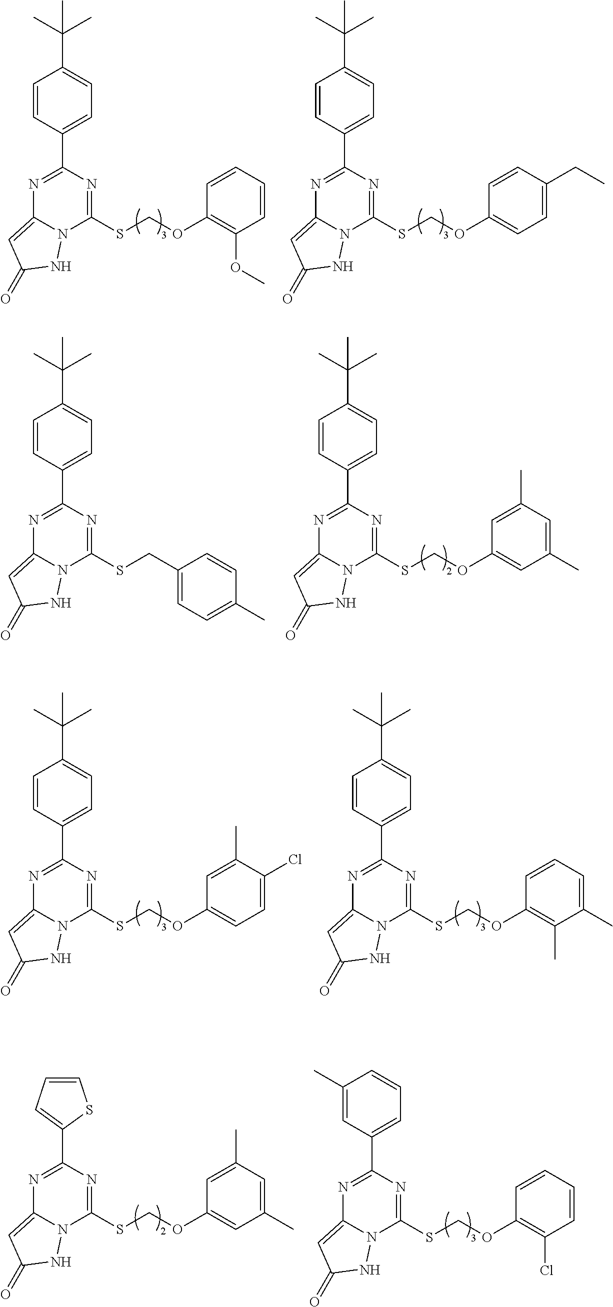

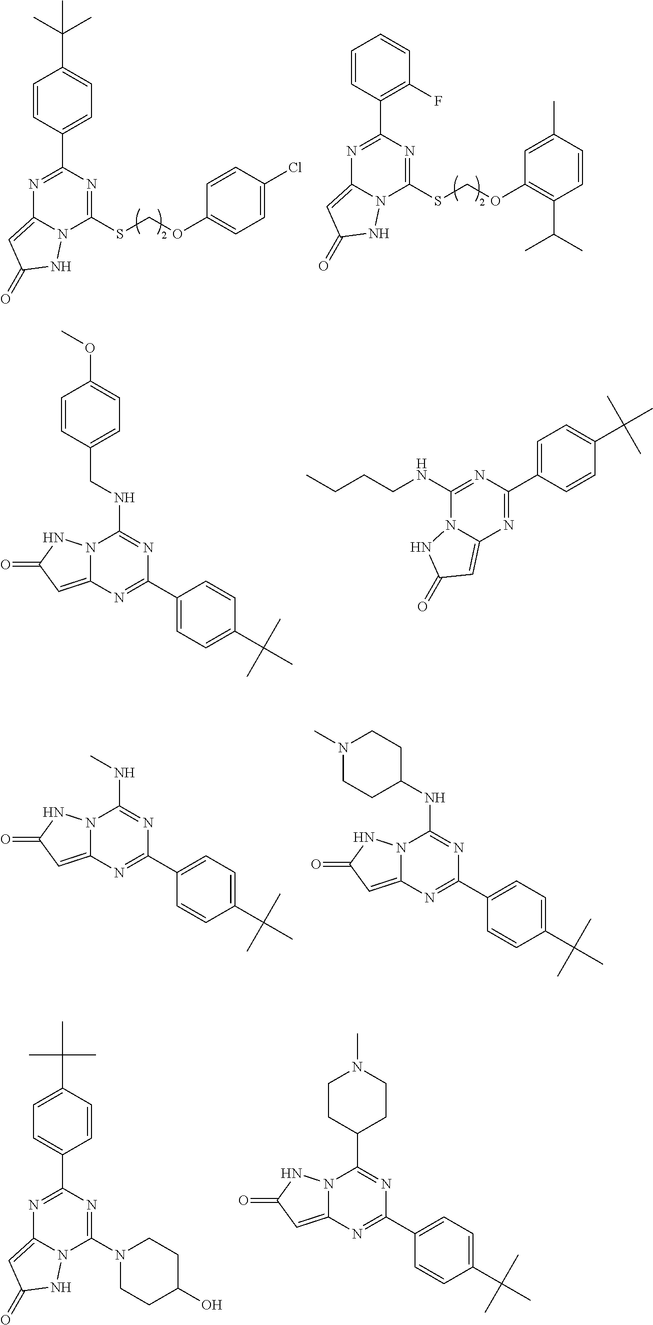

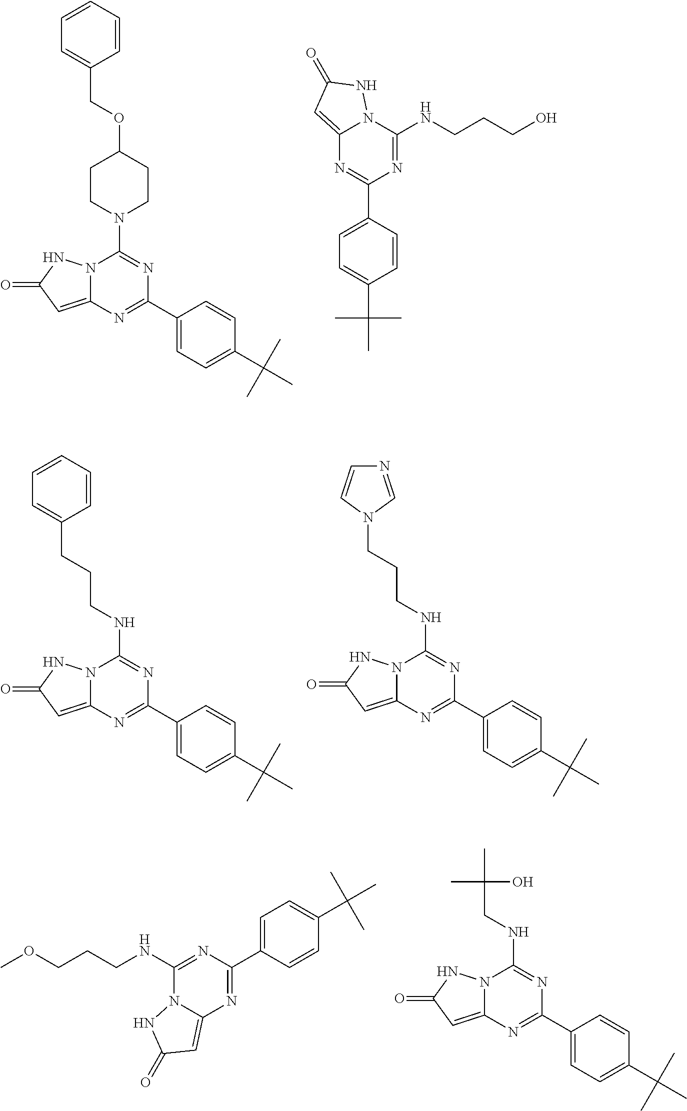 [SMILES: CC(C)(C)c1ccc(-c2nc(N3CCC(OCc4ccccc4)CC3)n3[nH]c(=O)cc3n2)cc1 ; CC(C)(C)c1ccc(-c2nc(NCCCO)n3[nH]c(=O)cc3n2)cc1 ; CC(C)(C)c1ccc(-c2nc(NCCCc3ccccc3)n3[nH]c(=O)cc3n2)cc1 ; CC(C)(C)c1ccc(-c2nc(NCCCn3ccnc3)n3[nH]c(=O)cc3n2)cc1 ; COCCCNc1nc(-c2ccc(C(C)(C)C)cc2)nc2cc(=O)[nH]n12 ; CC(C)(O)CNc1nc(-c2ccc(C(C)(C)C)cc2)nc2cc(=O)[nH]n12]

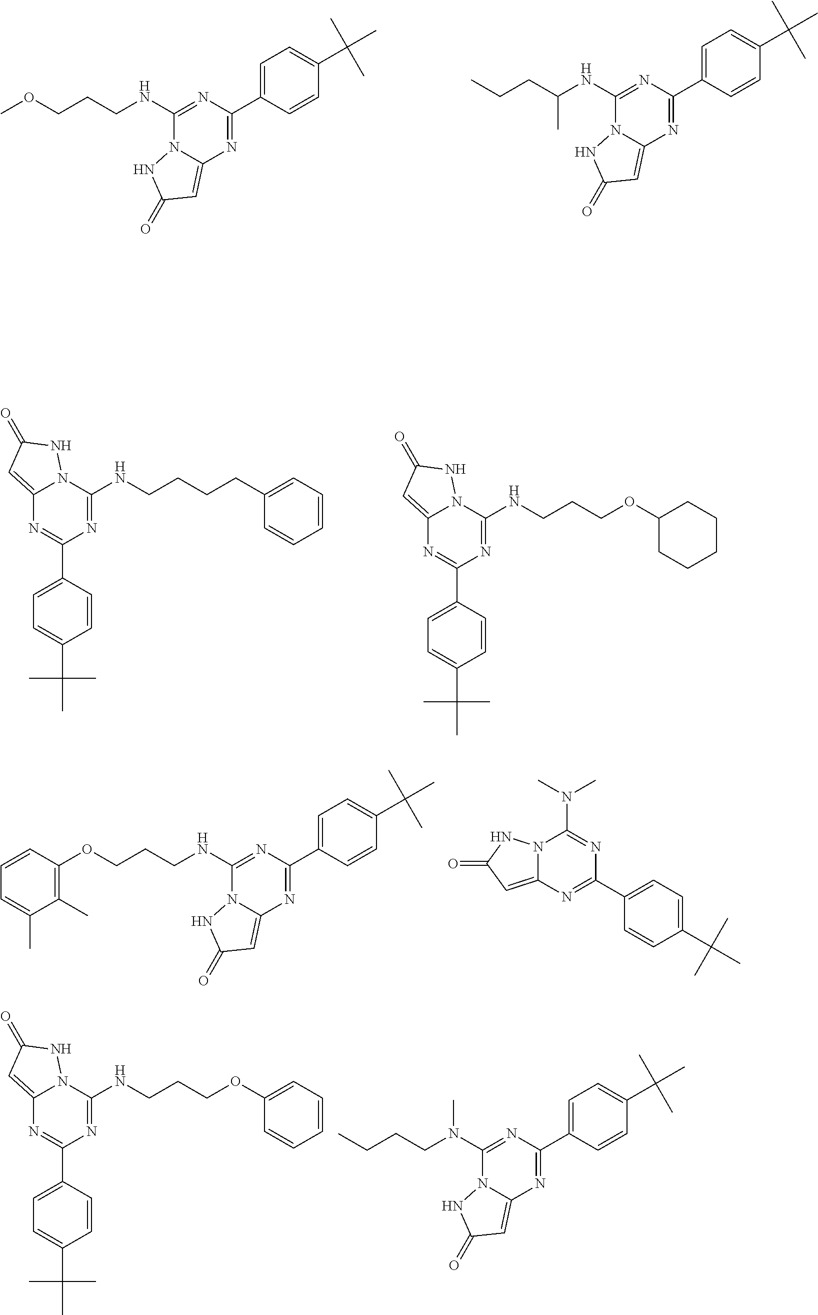

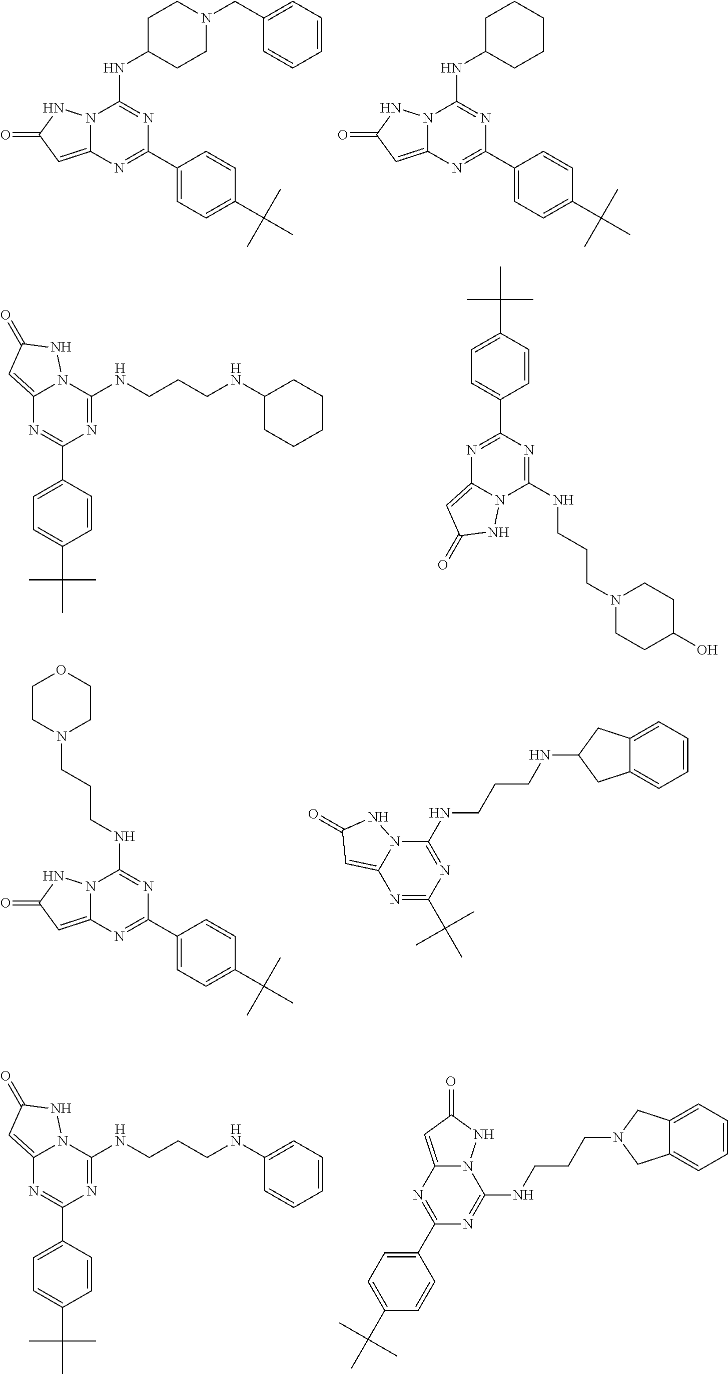

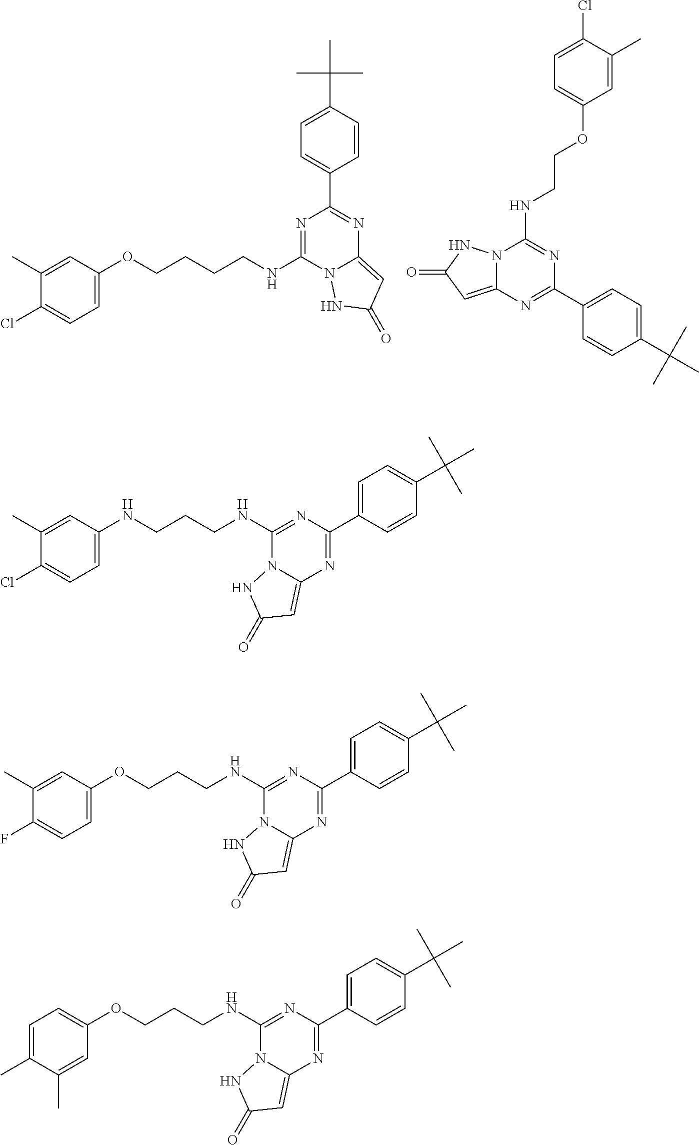

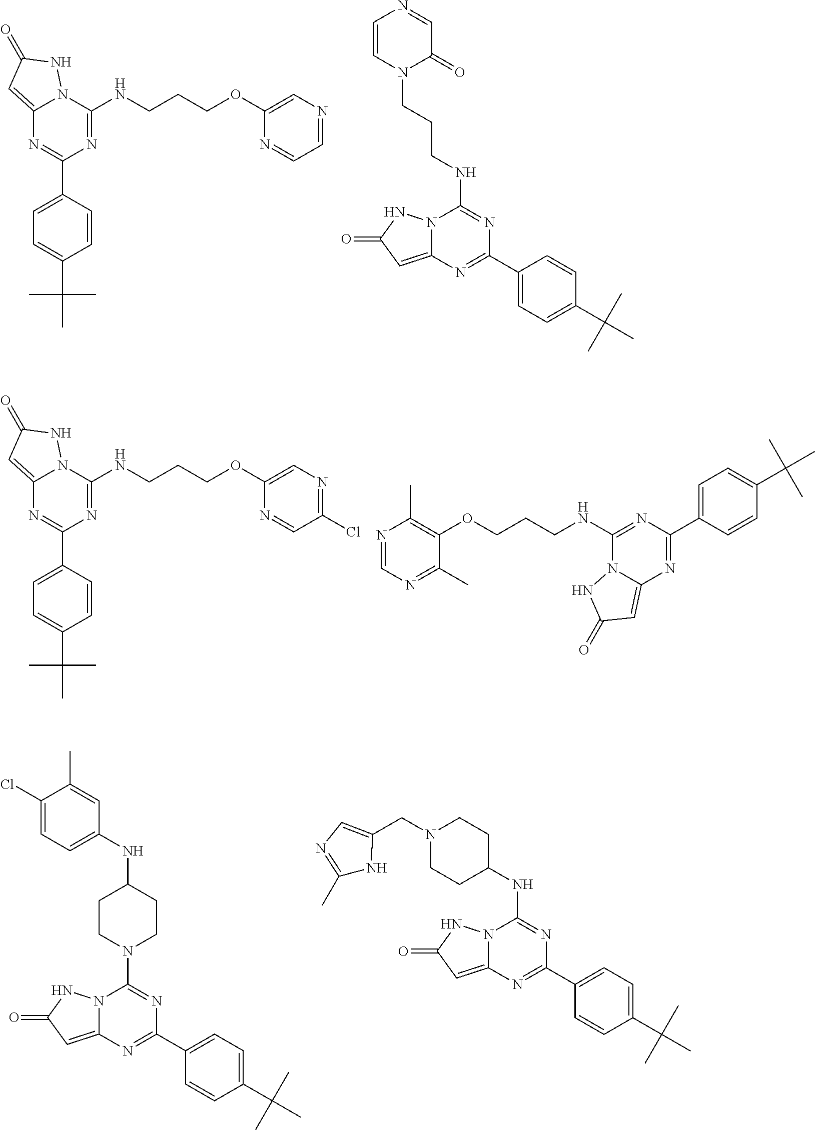

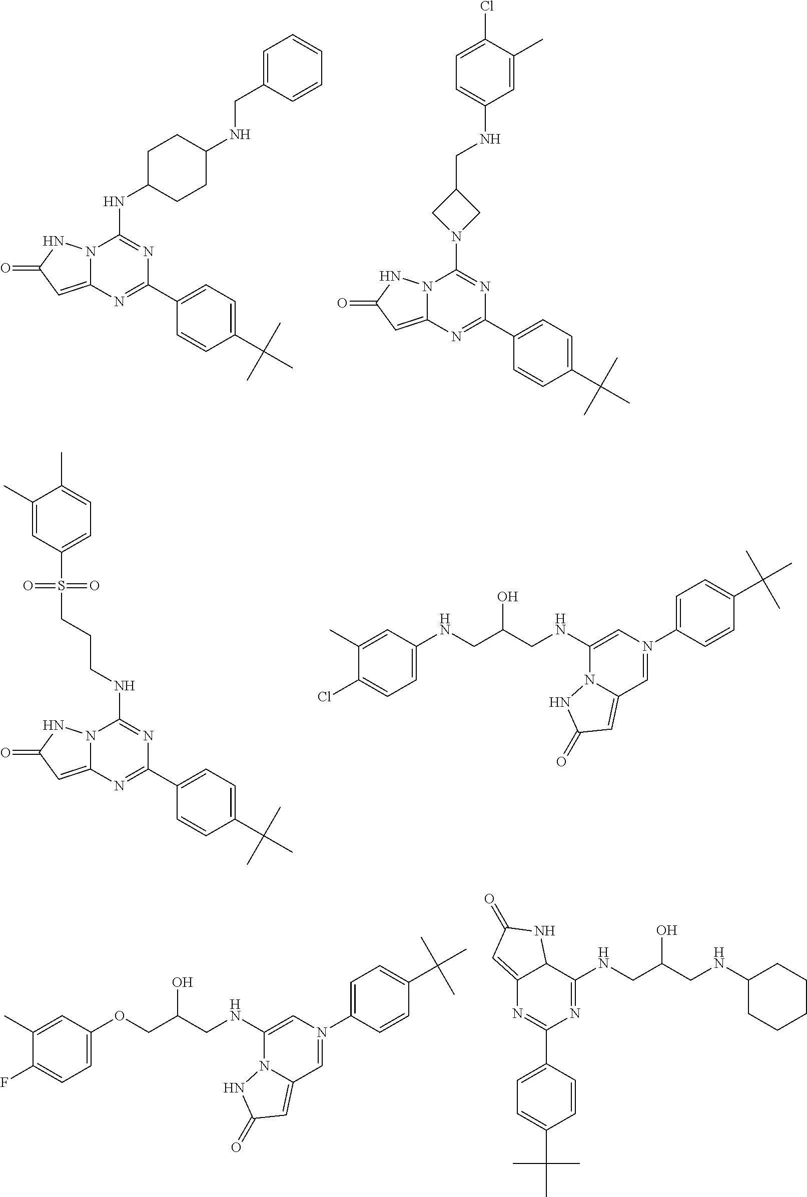

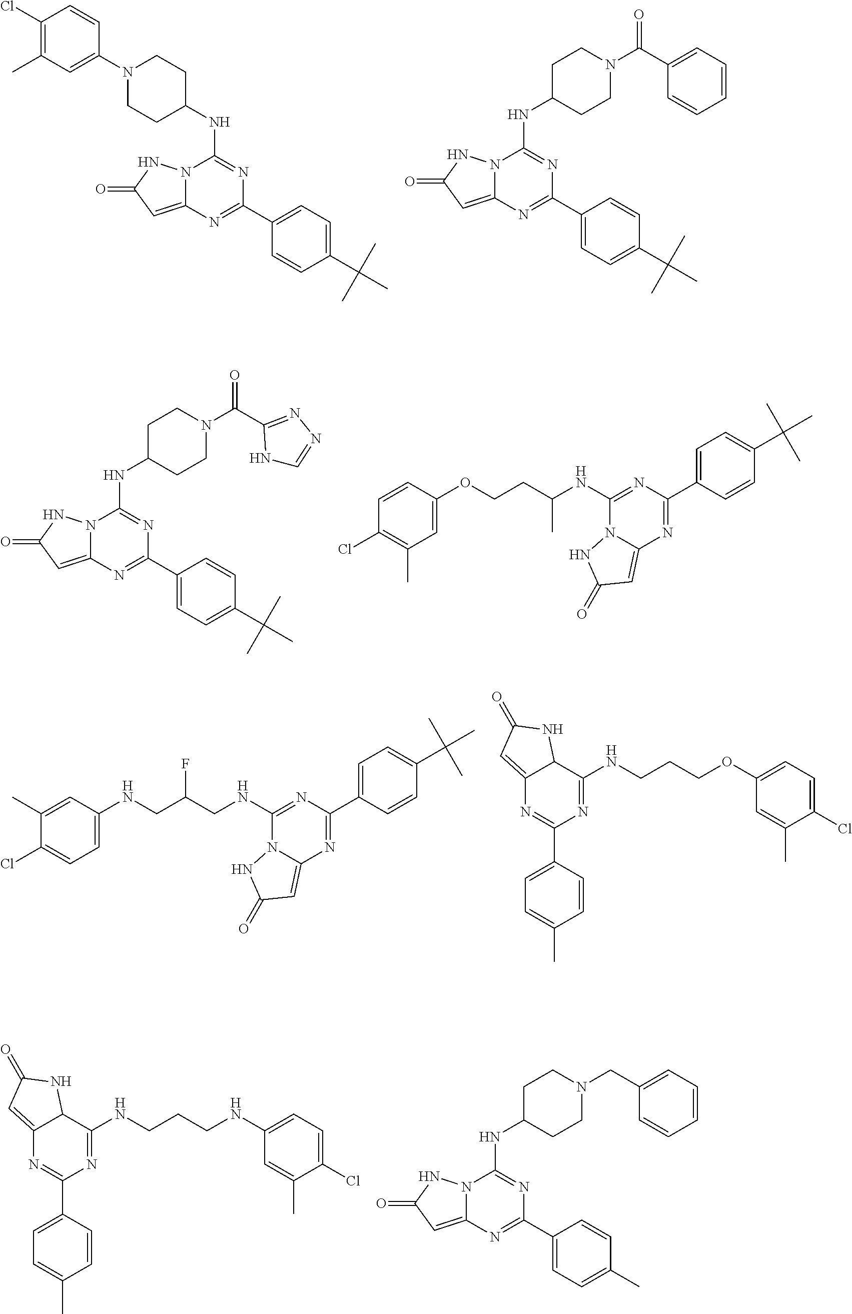

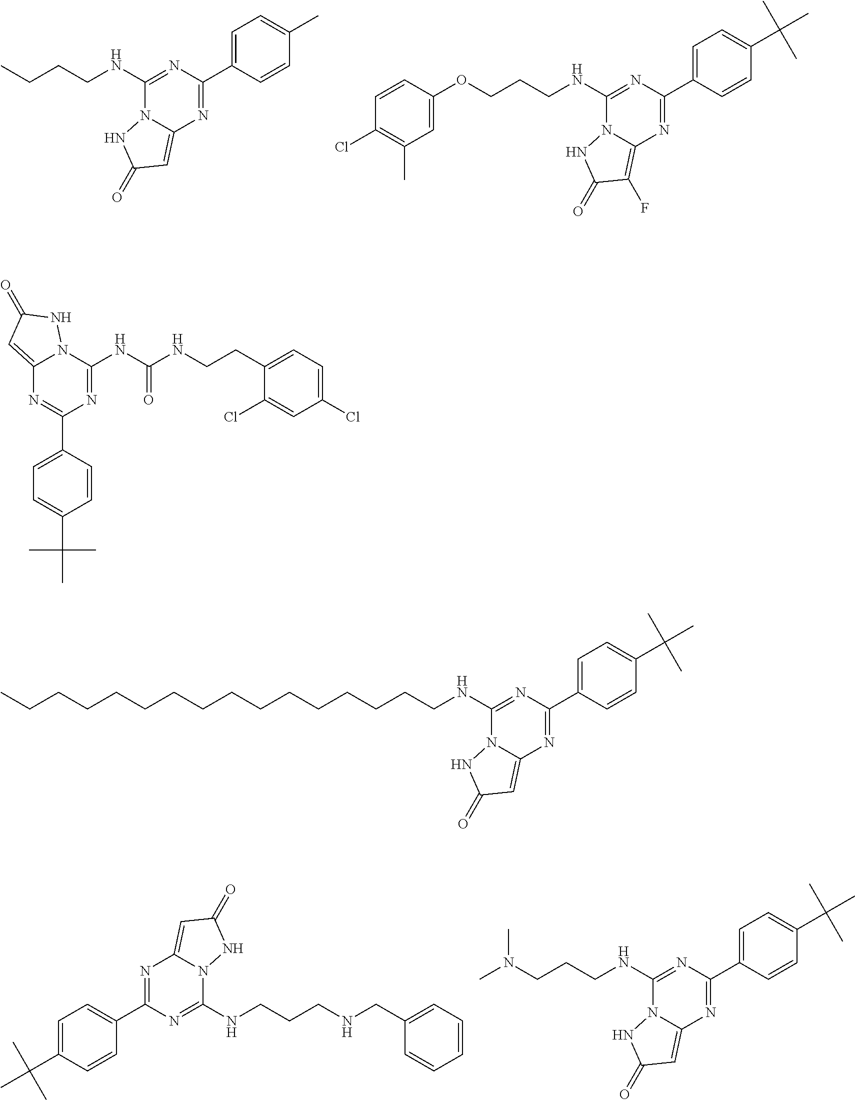

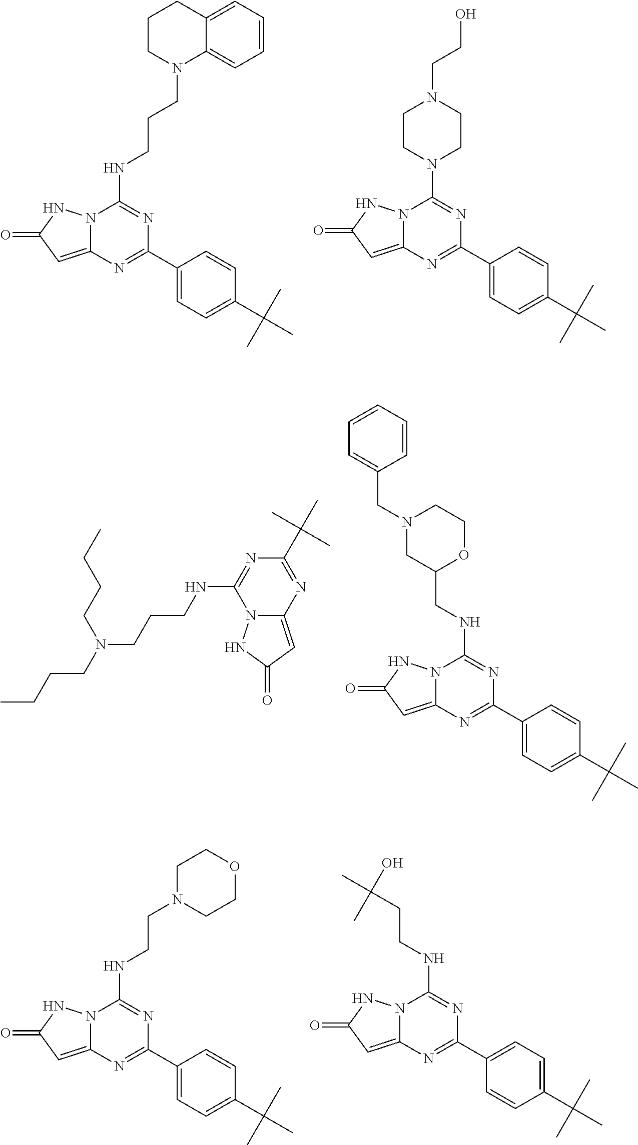 [SMILES: CC(C)(C)c1ccc(-c2nc(NCCCN3CCCc4ccccc43)n3[nH]c(=O)cc3n2)cc1 ; CC(C)(C)c1ccc(-c2nc(N3CCN(CCO)CC3)n3[nH]c(=O)cc3n2)cc1 ; CCCCN(CCCC)CCCNc1nc(C(C)(C)C)nc2cc(=O)[nH]n12 ; CC(C)(C)c1ccc(-c2nc(NCC3CN(Cc4ccccc4)CCO3)n3[nH]c(=O)cc3n2)cc1 ; CC(C)(C)c1ccc(-c2nc(NCCN3CCOCC3)n3[nH]c(=O)cc3n2)cc1 ; CC(C)(O)CCNc1nc(-c2ccc(C(C)(C)C)cc2)nc2cc(=O)[nH]n12]

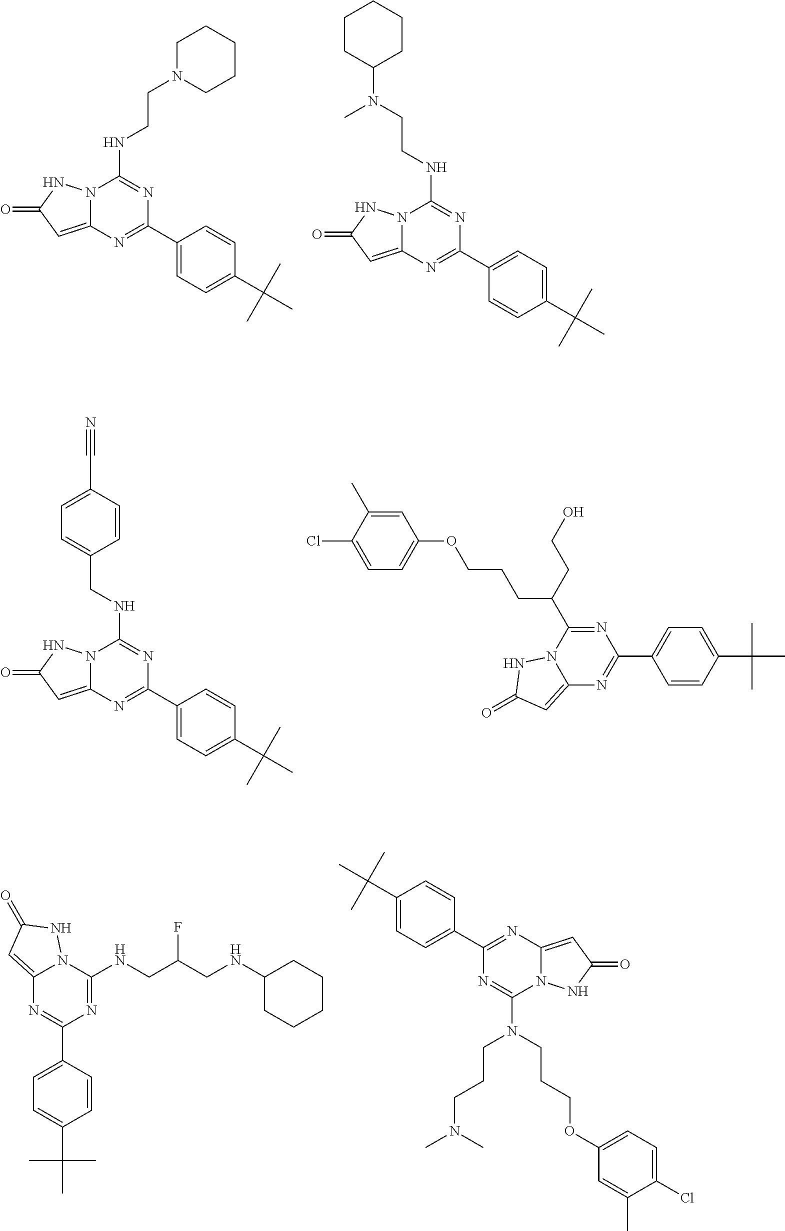 [SMILES: CC(C)(C)c1ccc(-c2nc(NCCN3CCCCC3)n3[nH]c(=O)cc3n2)cc1 ; CN(CCNc1nc(-c2ccc(C(C)(C)C)cc2)nc2cc(=O)[nH]n12)C1CCCCC1 ; CC(C)(C)c1ccc(-c2nc(NCc3ccc(C#N)cc3)n3[nH]c(=O)cc3n2)cc1 ; Cc1cc(OCCCC(CCO)c2nc(-c3ccc(C(C)(C)C)cc3)nc3cc(=O)[nH]n23)ccc1Cl ; CC(C)(C)c1ccc(-c2nc(NCC(F)CNC3CCCCC3)n3[nH]c(=O)cc3n2)cc1 ; Cc1cc(OCCCN(CCCN(C)C)c2nc(-c3ccc(C(C)(C)C)cc3)nc3cc(=O)[nH]n23)ccc1Cl]

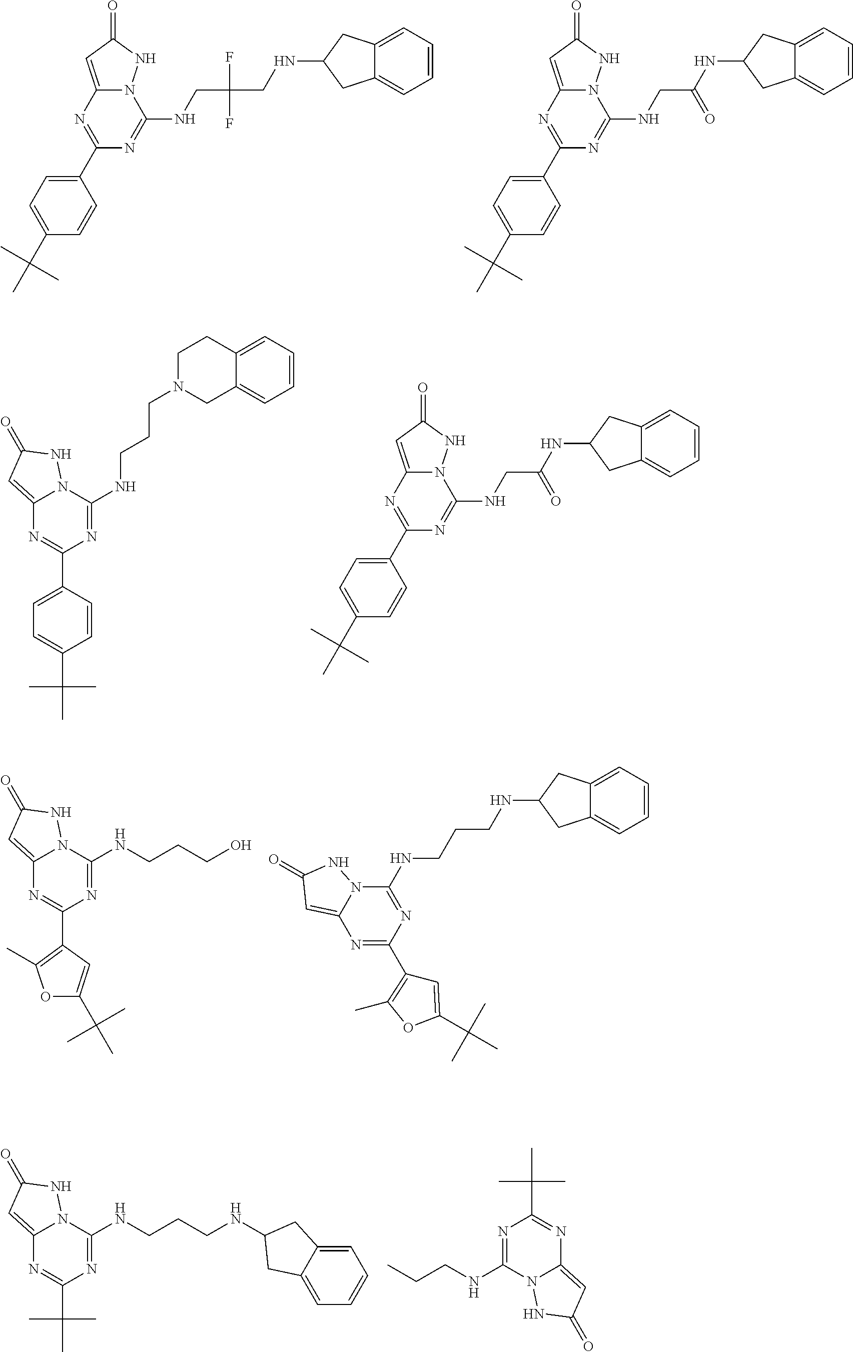

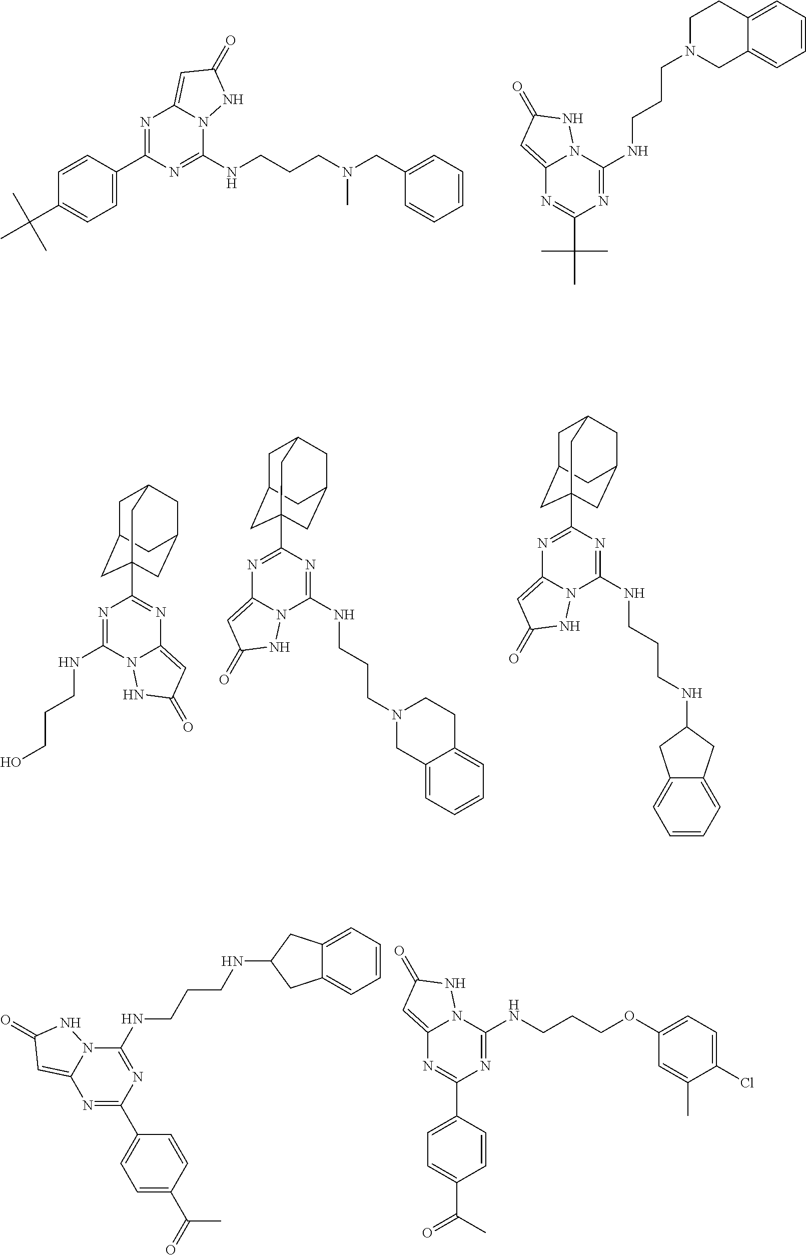

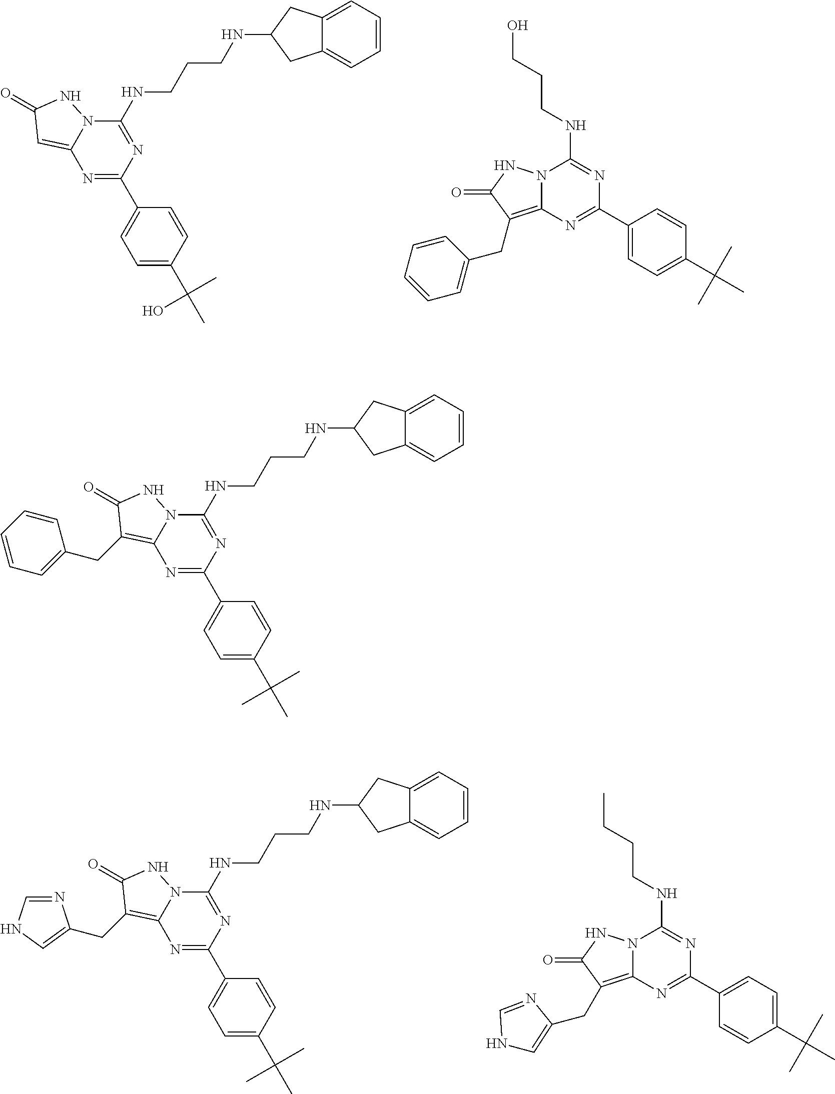 [SMILES: CC(C)(O)c1ccc(-c2nc(NCCCNC3Cc4ccccc4C3)n3[nH]c(=O)cc3n2)cc1 ; CC(C)(C)c1ccc(-c2nc(NCCCO)n3[nH]c(=O)c(Cc4ccccc4)c3n2)cc1 ; CC(C)(C)c1ccc(-c2nc(NCCCNC3Cc4ccccc4C3)n3[nH]c(=O)c(Cc4ccccc4)c3n2)cc1 ; CC(C)(C)c1ccc(-c2nc(NCCCNC3Cc4ccccc4C3)n3[nH]c(=O)c(Cc4c[nH]cn4)c3n2)cc1 ; CCCCNc1nc(-c2ccc(C(C)(C)C)cc2)nc2c(Cc3c[nH]cn3)c(=O)[nH]n12]

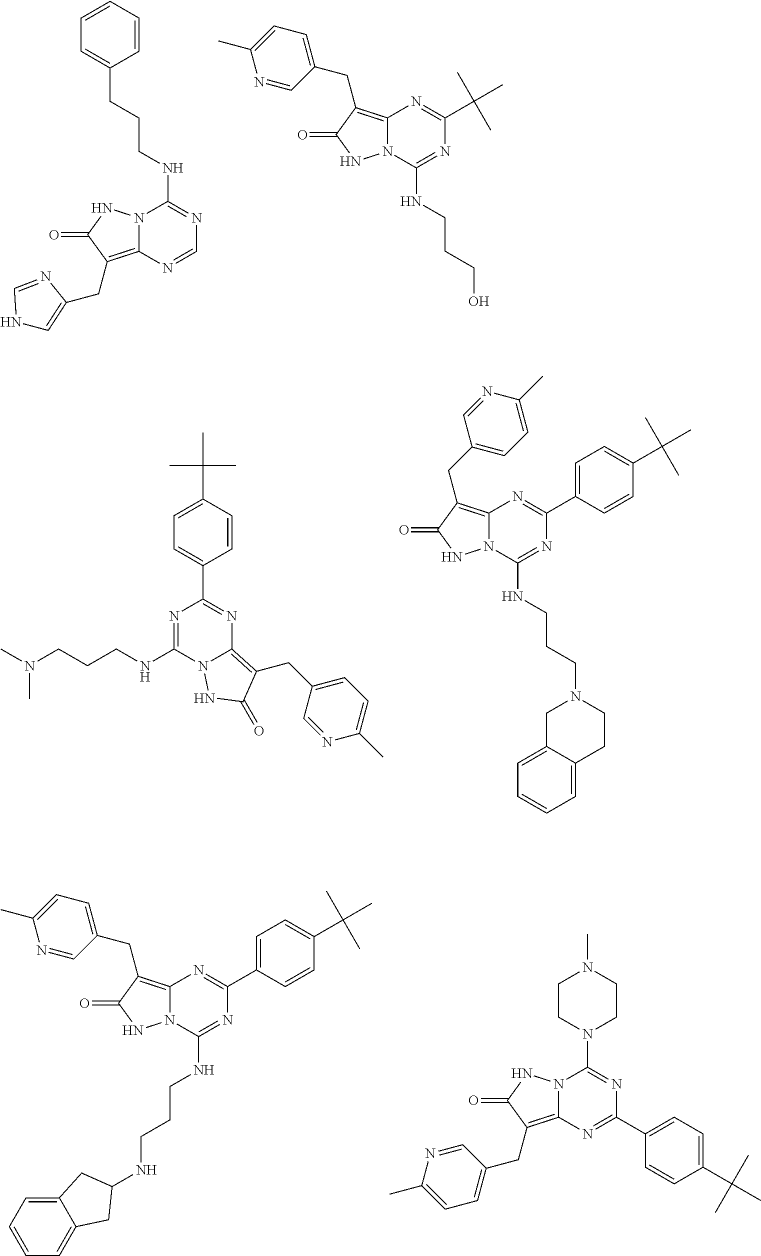

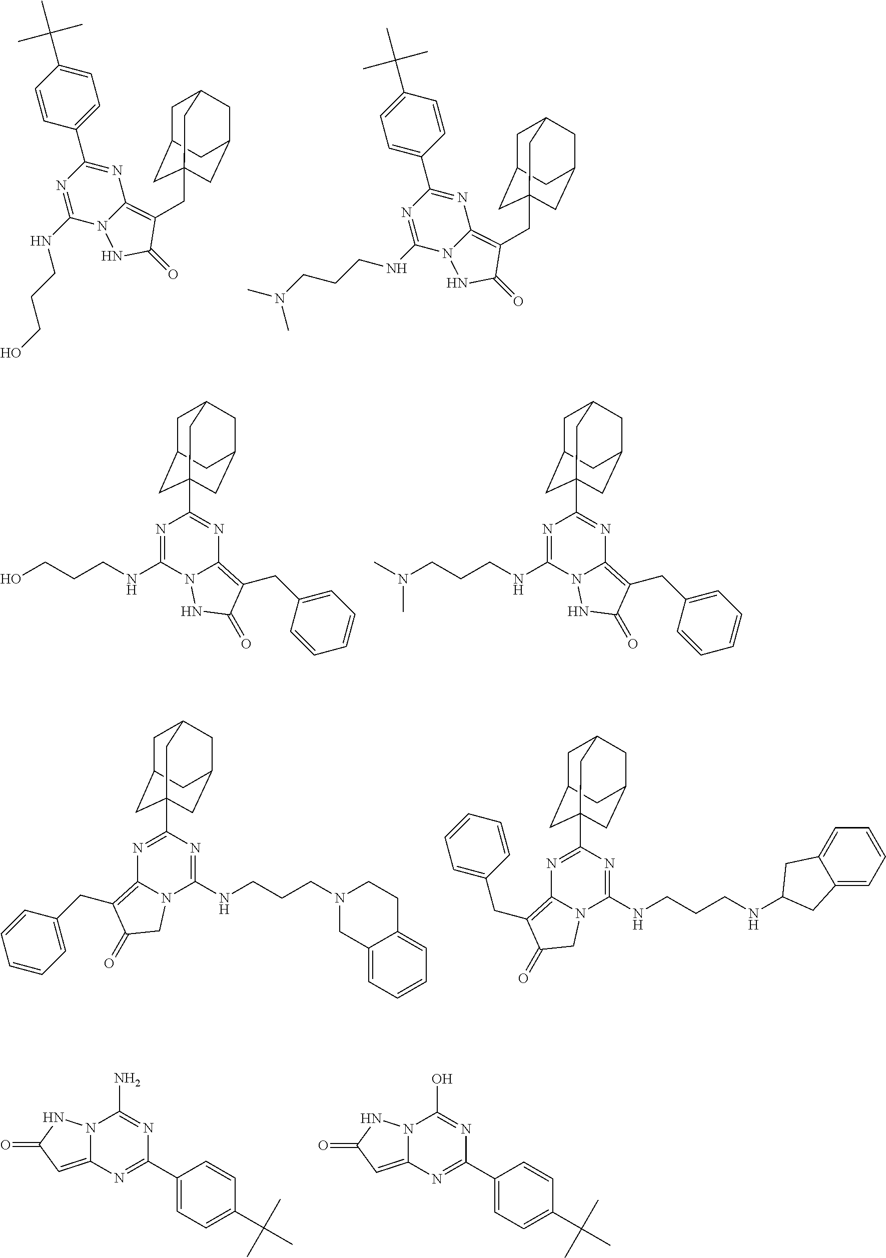

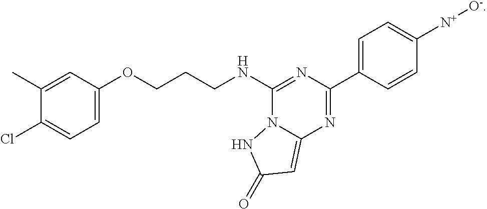

143. A compound of any one of claims 1-123, or a pharmaceutically acceptable salt, solvate, hydrate, polymorph, co-crystal, tautomer, stereoisomer, isotopically labeled derivative, or prodrug thereof, or a pharmaceutical composition of any one of claims 124-125, for use in treating and/or preventing a bacterial infection in a subject in need thereof.
